(19) United States
(12) Patent Application Publication
(10) Pub. No.: US 2025/0346922 A1
(43) Pub. Date: Nov. 13, 2025

(54) COMPOUNDS FOR IMPROVEMENT OF VIRAL PRODUCTION

(71) Applicant: Tenaya Therapeutics, Inc., South San Francisco, CA (US)

(72) Inventors: Frank Yifeng JING, Fremont, CA (US); Jackson LEONG, San Francisco, CA (US); Beatriz Jaimie LIM, Daly City, CA (US); Charles Jonathan FEATHERS, San Francisco, CA (US); Mohammad Ali MANDEGAR, South San Francisco, CA (US)

(21) Appl. No.: 19/177,253

(22) Filed: Apr. 11, 2025

Related U.S. Application Data
(63) Continuation of application No. PCT/US2023/076906, filed on Oct. 13, 2023.
(60) Provisional application No. 63/379,567, filed on Oct. 14, 2022.
(60) Provisional application No. 63/502,894, filed on May 17, 2023.

(51) Int. Cl. C12N 15/86

(52) U.S. Cl. CPC C12N 15/86; C12N 2750/14122; C12N 2750/14143; C12N 2750/14152

(57) ABSTRACT

Provided herein are methods of improving (e.g., increasing) the production of viral particles (e.g., AAV) in mammalian cells (e.g., HEK293 or HEK293T cells) using selective HDAC6 inhibitors (e.g., compounds of Formula I, Formula II, or Formula III). Certain methods described herein achieve significantly greater viral titer yield (e.g., 2 or more times greater yield) over the methods known in the art.

### CROSS-REFERENCE TO RELATED APPLICATIONS

This application is a Continuation of International Application No. PCT/US2023/076906, filed Oct. 13, 2023, which claims the priority to U.S. Provisional Patent Application No. 63/379,567, filed Oct. 14, 2022, and U.S. Provisional Patent Application No. 63/502,894, filed May 17, 2023, the contents of each of which are incorporated by reference herein in their entireties.

### REFERENCE TO SEQUENCE LISTING

The Sequence Listing associated with this application is provided electronically in XML file format and is hereby incorporated by reference into the specification in its entirety.

The name of the XML file containing the Sequence Listing is TENA_040_O₂US_SeqList_ST26.xml. The XML file is 175,600 bytes, was created on Apr. 11, 2025, and is being submitted electronically through the USPTO patent Center.

### TECHNICAL FIELD

The present disclosure relates generally to compounds and methods for improving production of viral particles (e.g., AAV) in mammalian cells.

### BACKGROUND

For successful clinical use of virus-based gene therapy, large-scale, high titer yield production of viral particles needs to be achieved. There is a need to improve viral production in cells to achieve higher titer yield than that achieved using known production methods.

Given the advantages of adeno-associated virus (AAV) gene therapy and other biomedical applications, there exists a particular need for improved method of production of AAV particles.

There remains a need in the art for improved methods of viral production.

### SUMMARY

In some aspects, the present disclosure provides a method of producing viral particles and/or increasing viral particle titer, comprising: transfecting or transducing cells in a cell culture with viral particles or a recombinant viral vector to be packaged in the cells into viral particles, adding to the cell culture a selective HDAC6 inhibitor, and culturing the cells in the cell culture.

In some embodiments, the cells in the cell culture are mammalian cells. In some embodiments, the mammalian cells are selected from the group consisting of the following cells or cell lines: HEK293, HEK293T, HeLa, Vero, MDCK, MRC-5, PER.C6, BHK21 and CHO. In some embodiments, the mammalian cells are HEK293 or HEK293T cells or cell lines.

In some embodiments, the virus in the viral particles or recombinant viral vector is an adenovirus, an adeno-associated virus (AAV), a lentivirus, a retrovirus, a herpes virus, a herpes simplex virus, a vaccinia virus, an influenza virus, a rotavirus, a Hepatitis A virus, a CMV virus, an RSV virus, a rotavirus, a vesicular stomatitis virus, or a rabies virus. In some embodiments, the virus is an AAV virus. In some embodiments, the virus is a lentivirus. In some embodiments, the virus is a retrovirus.

In some embodiments, the selective HDAC6 inhibitor is at least 50-fold or at least 100-fold more selective against HDAC6 compared to all other isozymes of HDAC. In some embodiments, the selective HDAC6 inhibitor is at least 500-fold or at least 1000-fold more selective against HDAC6 compared to all other isozymes of HDAC.

In some embodiments, the selective HDAC6 inhibitor is a fluoroalkyl-oxadiazole derivative.

In some aspects, the selective HDAC6 inhibitor is a compound Formula (I):

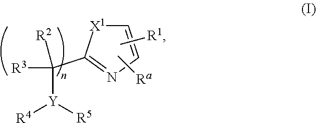

or a salt thereof, wherein
R¹ is selected from the group consisting of:

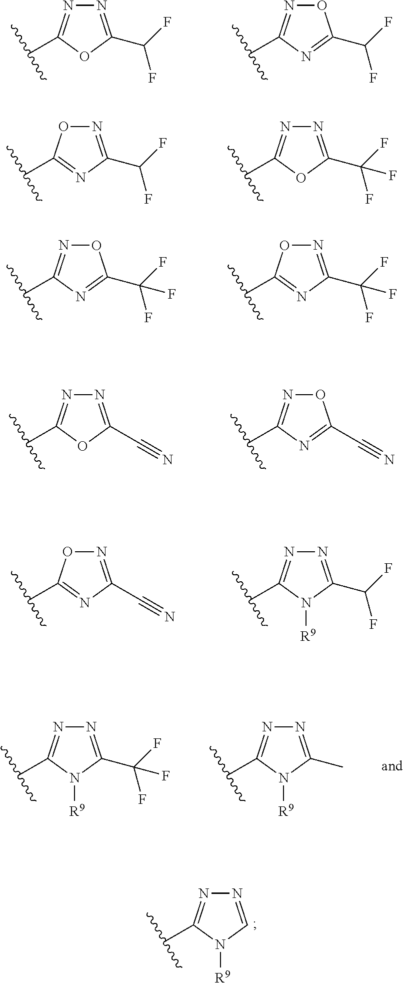

R^{a} is selected from the group consisting of H, halo, C₁₋₃ alkyl, cycloalkyl, haloalkyl, and alkoxy;
R² and R³ are independently selected from the group consisting of H, halogen, alkoxy, haloalkyl, aryl, heteroaryl, alkyl, and cycloalkyl each of which is optionally substituted, or R² and R³ together with the atom to which they are attached form a cycloalkyl or heterocyclyl;
R⁴ and R⁵ are independently selected from the group consisting of H, —(SO₂)R², —(SO₂)NR²R³, —(CO)R², —(CONR²R³), aryl, arylheteroaryl, alkylenearyl, heteroaryl, cycloalkyl, heterocyclyl, alkyl, haloalkyl, and alkoxy, each of which is optionally substituted, or R⁴ and R⁵ together with the atom to which they are attached form a cycloalkyl or heterocyclyl, each of which is optionally substituted;
R⁹ is selected from the group consisting of H, C₁-C₆ alkyl, haloalkyl, cycloalkyl and heterocyclyl;
X¹ is selected from the group consisting of S, O, NH and NR⁶, wherein R⁶ is selected from the group consisting of C₁-C₆ alkyl, alkoxy, haloalkyl, cycloalkyl and heterocyclyl;
Y is selected from the group consisting of CR², O, N, S, SO, and SO₂, wherein when Y is O, S, SO, or SO₂, R⁵ is not present and when R⁴ and R⁵ together with the atom to which they are attached form a cycloalkyl or heterocyclyl, Y is CR² or N; and
n is selected from 0, 1, and 2.

In some aspects, the selective HDAC6 inhibitor is a compound having the formula:

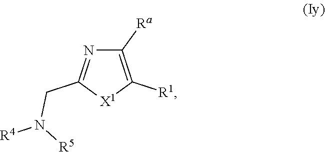

or a salt thereof, wherein:
X¹ is S;
R^{a} is selected from the group consisting of H, halogen, and C₁₋₃ alkyl;
R¹ is

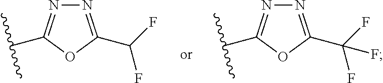 [SMILES: *c1nnc(C(F)F)o1 ; *c1nnc(C(F)(F)F)o1]

R² is selected from the group consisting of alkyl, alkoxy, and cycloalkyl, each of which is optionally substituted;
R³ is H or alkyl;
R⁴ is selected from the group consisting of alkyl, —(SO₂)R², —(SO₂)NR²R³, and —(CO)R²; and
R⁵ is aryl or heteroaryl; or R⁴ and R⁵ together with the atom to which they are attached form a heterocyclyl, each of which is optionally substituted.

In some embodiments, R¹ is

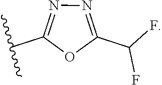 [SMILES: *c1nnc(C(F)F)o1]

In some aspects, the selective HDAC6 inhibitor is a compound of Formula (Ic):

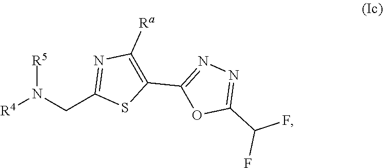

or a salt thereof, wherein:
R^{a} is selected from the group consisting of H, halo, C₁₋₃ alkyl, cycloalkyl, haloalkyl, and alkoxy;
R² is selected from the group consisting of alkyl, alkoxy, and cycloalkyl, each of which is optionally substituted;
R³ is H, alkyl, or aryl;
R⁴ selected from the group consisting of H, —(SO₂)R², —(SO₂)NR²R³, —(CO)R², —(CONR²R³), aryl, arylheteroaryl, alkylenearyl, heteroaryl, cycloalkyl, heterocyclyl, alkyl, haloalkyl, and alkoxy, each of which is optionally substituted;
R⁵ is aryl, arylheteroaryl, alkylenearyl, heteroaryl, cycloalkyl, heterocyclyl, alkyl, haloalkyl, and alkoxy, each of which is optionally substituted; or R⁴ and R⁵ together with the atom to which they are attached form a heterocyclyl, each of which is optionally substituted.
R⁹ is selected from the group consisting of H, C₁-C₆ alkyl, haloalkyl, cycloalkyl and heterocyclyl.

In some embodiments, R^{a} is H.

In some embodiments, R⁴ is —(SO₂)R². In some embodiments, —(SO₂)R² is —(SO₂)alkyl, —(SO₂)alkyleneheterocyclyl, —(SO₂)haloalkyl, —(SO₂)haloalkoxy, or —(SO₂)cycloalkyl.

In some embodiments, R⁵ is heteroaryl. In some embodiments, the heteroaryl is a 5- to 6-membered heteroaryl. In some embodiments, the 5- to 6-membered heteroaryl is selected from the group consisting of

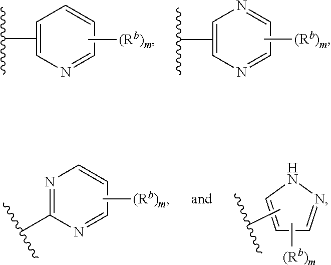

wherein R^{b} is halogen, alkyl, alkoxy, cycloalkyl, —CN, haloalkyl, or haloalkoxy; and mis 0 or 1.

In some embodiments, R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments, R⁵ is aryl. In some embodiments, the aryl is selected from the group consisting of phenyl, 3-chlorophenyl, 3-chloro-4-fluorophenyl, 3-trifluoromethylphenyl, 3,4-difluorophenyl, and 2,6-difluorophenyl.

In some aspects, the selective HDAC6 inhibitor is a compound of Formula (Ik):

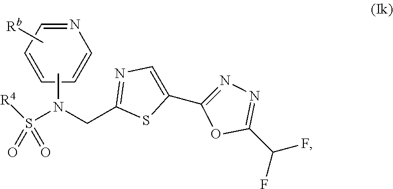

or a salt thereof, wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

In some embodiments, the compound of Formula (Ik) is a compound having the structure:

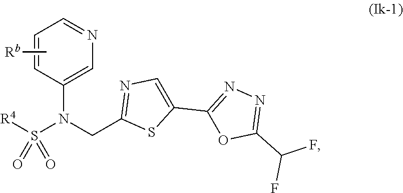

or a salt thereof.

In some embodiments, the compound of Formula (Ik) is a compound having the structure:

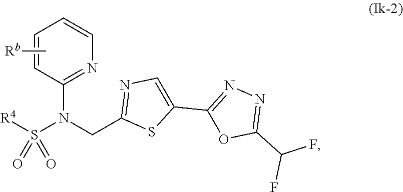

or a salt thereof.

In some embodiments of Formulas (Ik), (Ik-1), and (Ik-2), R^{b} is halogen, alkyl, alkoxy, cycloalkyl, —CN, haloalkyl, or haloalkoxy. In some embodiments, R^{b} is H, halogen, haloalkyl, or haloalkoxy. In some embodiments, R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments of Formulas (Ik), (Ik-1), and (Ik-2), R⁴ is optionally substituted alkyl or cycloalkyl. In some embodiments, R⁴ is optionally substituted alkyl. In some embodiments, R⁴ is alkyl.

In some embodiments, the selective HDAC6 inhibitor is a compound of Formula:

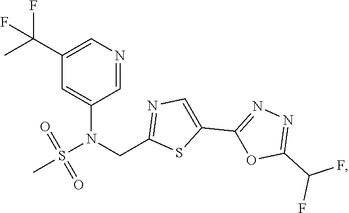 [SMILES: CC(F)(F)c1cncc(N(Cc2ncc(-c3nnc(C(F)F)o3)s2)S(C)(=O)=O)c1]

or an analog or salt thereof.

In some embodiments, the selective HDAC6 inhibitor is a compound of Formula:

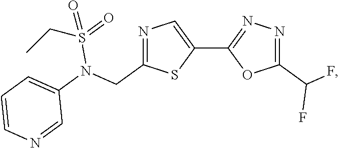 [SMILES: CCS(=O)(=O)N(Cc1ncc(-c2nnc(C(F)F)o2)s1)c1cccnc1]

or an analog or salt thereof.

In some embodiments, the selective HDAC6 inhibitor is a compound of Formula:

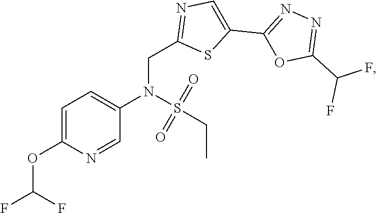 [SMILES: CCS(=O)(=O)N(Cc1ncc(-c2nnc(C(F)F)o2)s1)c1ccc(OC(F)F)nc1]

or an analog or salt thereof.

In some embodiments, the selective HDAC6 inhibitor is a compound of Formula:

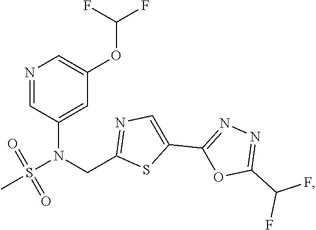 [SMILES: CS(=O)(=O)N(Cc1ncc(-c2nnc(C(F)F)o2)s1)c1cncc(OC(F)F)c1]

or an analog or salt thereof.

In some embodiments, the selective HDAC6 inhibitor is a compound of Formula:

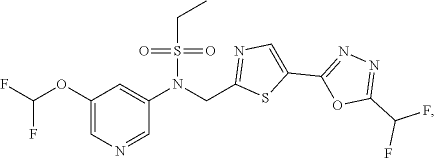 [SMILES: CCS(=O)(=O)N(Cc1ncc(-c2nnc(C(F)F)o2)s1)c1cncc(OC(F)F)c1]

or an analog or salt thereof.

In some embodiments, the selective HDAC6 inhibitor is a compound of Formula:

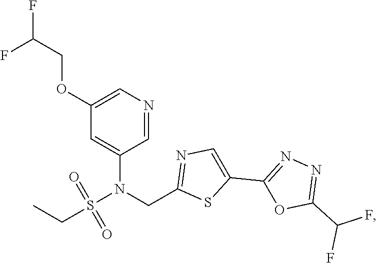 [SMILES: CCS(=O)(=O)N(Cc1ncc(-c2nnc(C(F)F)o2)s1)c1cncc(OCC(F)F)c1]

or an analog or salt thereof.

In some embodiments, the selective HDAC6 inhibitor is a compound of Formula:

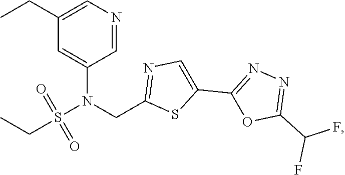 [SMILES: CCc1cncc(N(Cc2ncc(-c3nnc(C(F)F)o3)s2)S(=O)(=O)CC)c1]

or an analog or salt thereof.

In some embodiments, the selective HDAC6 inhibitor is a compound of Formula:

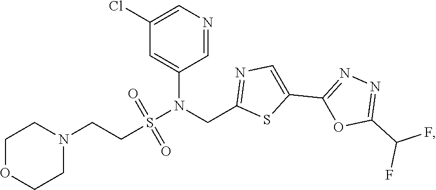 [SMILES: O=S(=O)(CCN1CCOCC1)N(Cc1ncc(-c2nnc(C(F)F)o2)s1)c1cncc(Cl)c1]

or an analog or salt thereof.

In some embodiments, the selective HDAC6 inhibitor is a compound of Formula:

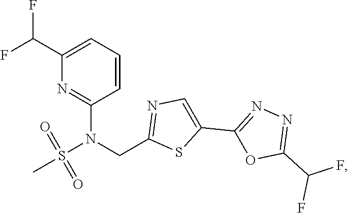 [SMILES: CS(=O)(=O)N(Cc1ncc(-c2nnc(C(F)F)o2)s1)c1cccc(C(F)F)n1]

or an analog or salt thereof.

In some embodiments, the selective HDAC6 inhibitor is a compound of Formula:

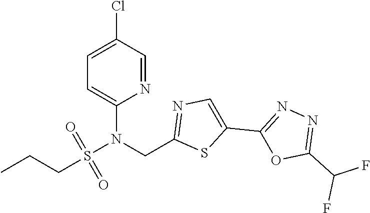 [SMILES: CCCS(=O)(=O)N(Cc1ncc(-c2nnc(C(F)F)o2)s1)c1ccc(Cl)cn1]

or an analog or salt thereof.

In some embodiments, the selective HDAC6 inhibitor is a compound of Formula:

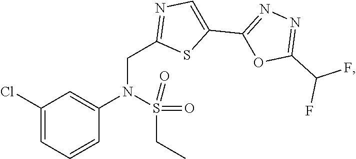 [SMILES: CCS(=O)(=O)N(Cc1ncc(-c2nnc(C(F)F)o2)s1)c1cccc(Cl)c1]

or an analog or salt thereof.

In some aspects, the selective HDAC6 inhibitor is a compound of Formula (II):

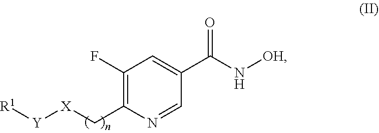

or a salt thereof,
wherein:
   n is 0 or 1;
   X is O, NR⁴, or CR⁴R^{4′};
   Y is a bond, CR²R³ or S(O)₂;
   R¹ is selected from the group consisting of H, amido, carbocyclyl, heterocyclyl, aryl, and heteroaryl;
   R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
   R¹ and R² taken together with the carbon atom to which they are attached form a carbocyclyl or heterocyclyl; or
   R² and R³ taken together with the carbon atom to which they are attached form a carbocyclyl or heterocyclyl; and
   R⁴ and R^{4′} are each independently selected from the group consisting of H, alkyl, —CO₂-alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
   R⁴ and R^{4′} taken together with the carbon atom to which they are attached form a carbocyclyl or heterocyclyl;
wherein each alkyl, carbocyclyl, heterocyclyl, aryl, and heteroaryl is independently optionally substituted with one or more substituents selected from the group consisting of halogen, haloalkyl, oxo, hydroxy, alkoxy, —OCH₃, —CO₂CH₃, —C(O)NH(OH), —CH₃, morpholine, and —C(O)N-cyclopropyl.

In some embodiments, the compound of Formula (II) is selected from the group consisting of:

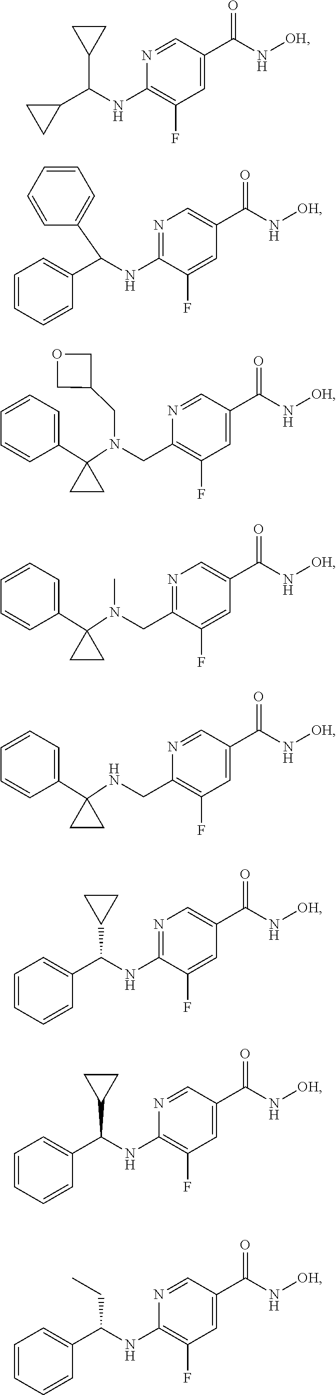

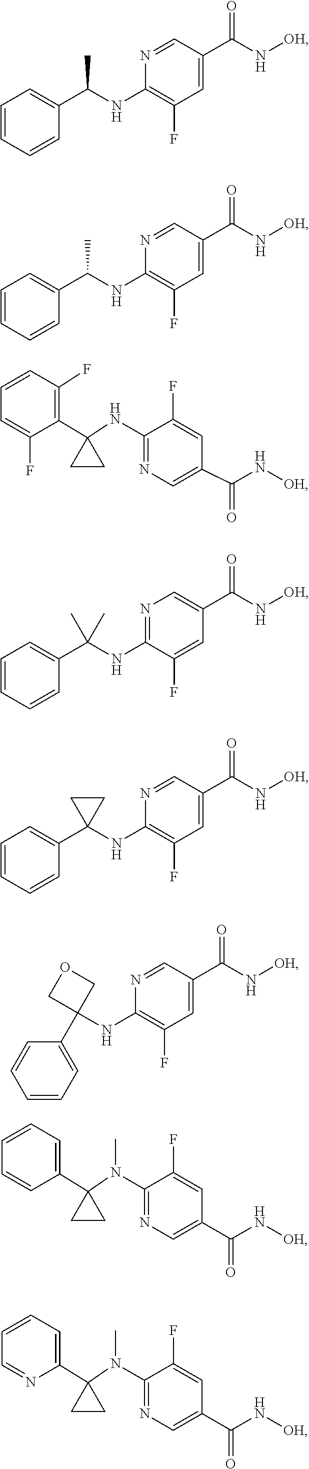

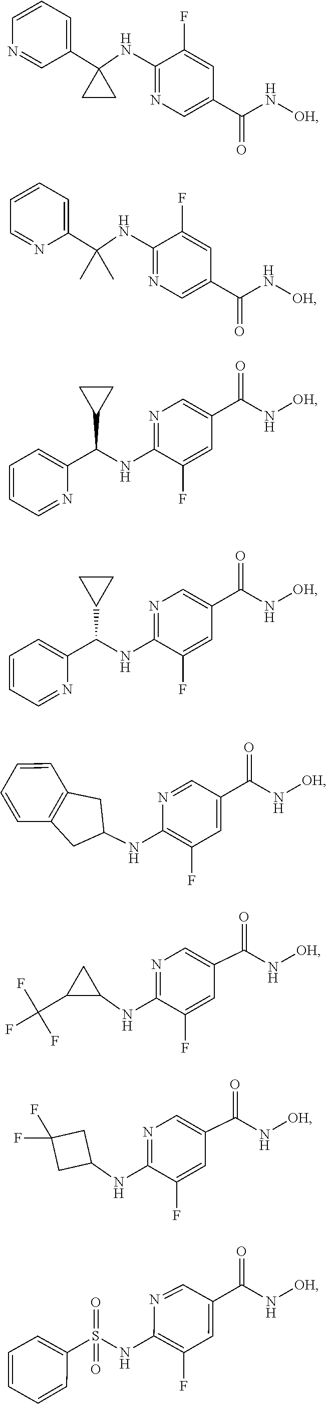

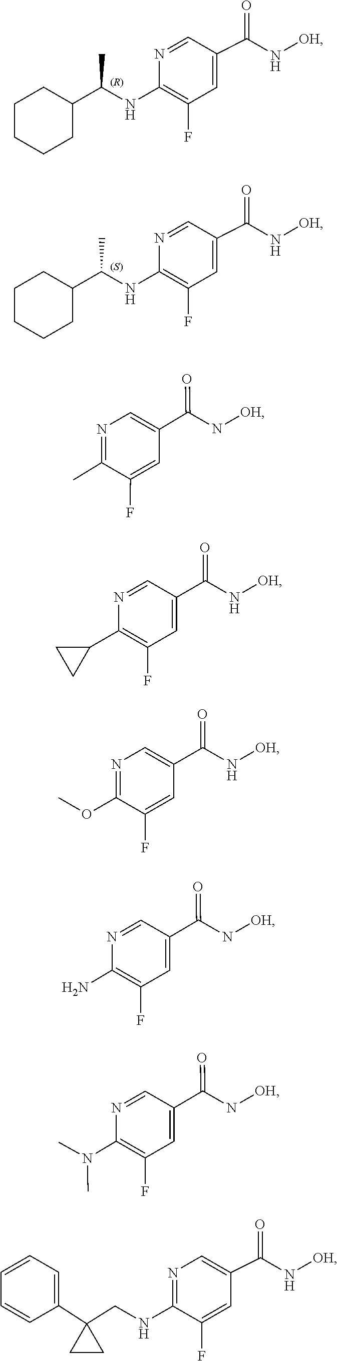

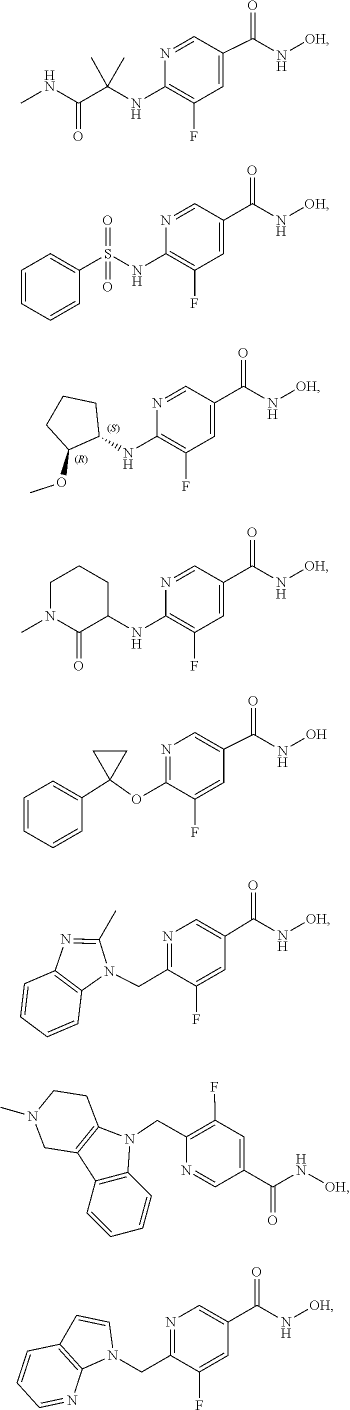

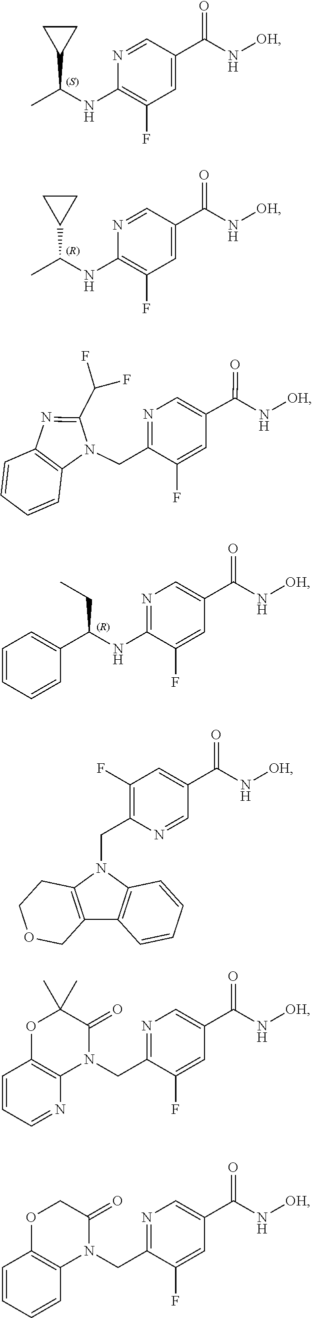

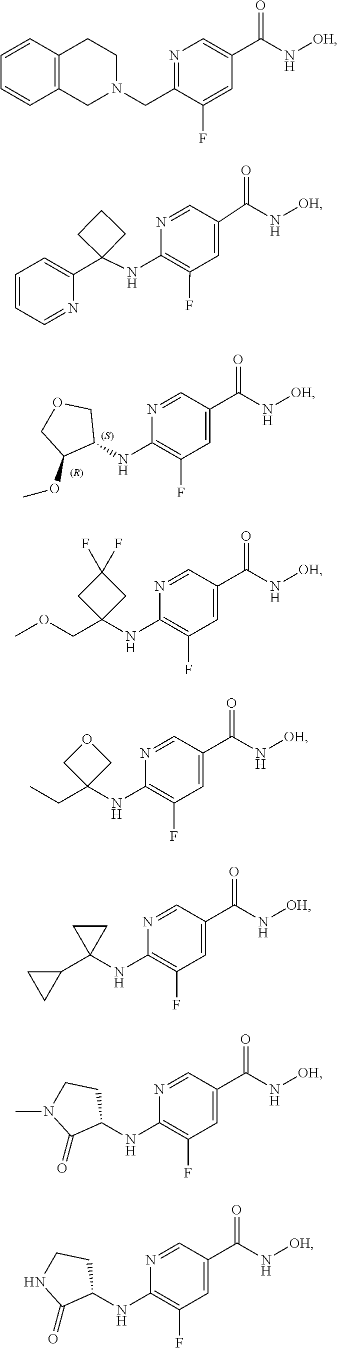

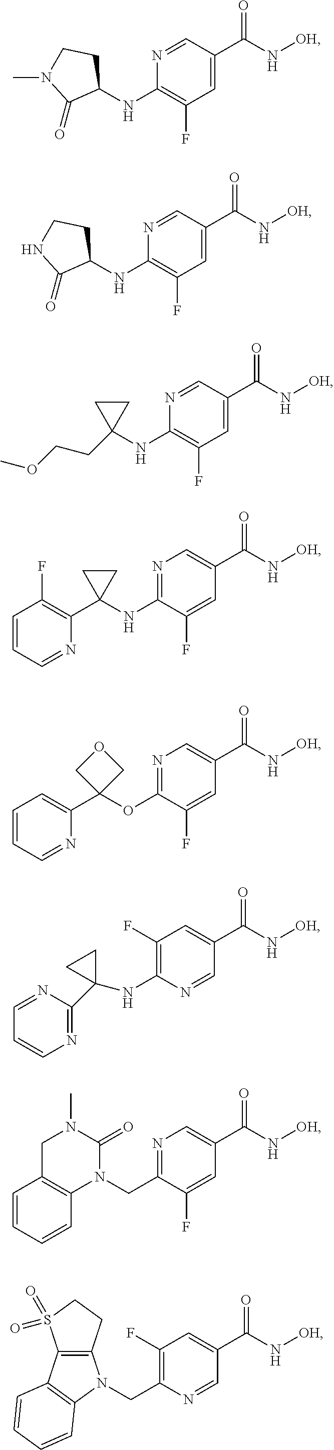

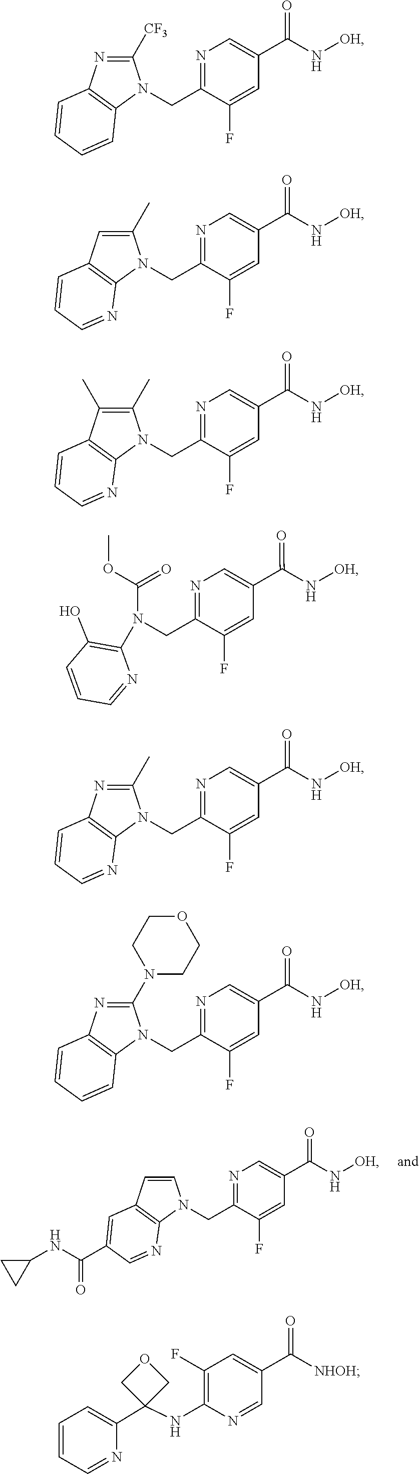

or an analog or salt thereof.

In some embodiments, the selective HDAC6 inhibitor is a compound of Table 2, Table 3, Table 4, or Table 5. In some embodiments, the selective HDAC6 inhibitor is a compound of Table 2, Table 3, Table 4, or Table 5, which has an IC₅₀ equal to or less than 0.03 μM. In some embodiments, the selective HDAC6 inhibitor is a compound of Table 2, e.g., which has an IC₅₀ equal to or less than 0.03 μM. In some embodiments, the selective HDAC6 inhibitor is a compound of Table 3, e.g., which has an IC₅₀ equal to or less than 0.03 μM. In some embodiments, the selective HDAC6 inhibitor is a compound of Table 4, e.g., which has an IC₅₀ equal to or less than 0.03 μM. In some embodiments, the selective HDAC6 inhibitor is a compound of Table 5, e.g., which has an IC₅₀ equal to or less than 0.03 μM.

In some embodiments, the selective HDAC6 inhibitor is not a hydroxamic acid. In some embodiments, the selective HDAC6 inhibitor is not rocilinostat.

In some embodiments, the selective HDAC6 inhibitor is added to the cell culture in an amount effective to achieve the concentration in the cell culture of between 0.1 μM and 100 μM. In some embodiments, the concentration of the selective HDAC6 inhibitor in the cell culture is between about 0.2 μM and 50 μM. In some embodiments, the selective HDAC6 inhibitor in the cell culture is between about 0.2 μM and 50 μM. In some embodiments, the concentration of the selective HDAC6 inhibitor in the cell culture is between about 0.5 μM and 15 μM. In some embodiments, the selective HDAC6 inhibitor in the cell culture is between about 1 μM and 5 μM.

In some embodiments, the selective HDAC6 inhibitor is added to the cell culture from 7 days before to 7 days after the transfection or transduction. In some embodiments, the selective HDAC6 inhibitor is added to the cell culture at least 1, 2, 3, 4, 5, 6 or 7 days after the transfection or transduction. In some embodiments, the selective HDAC6 inhibitor is added to the cell culture from 3 days before to 3 days after the transfection or transduction. In some embodiments, the selective HDAC6 inhibitor is added to the cell culture from 1 day before to 1 day after the transfection or transduction. In some embodiments, the selective HDAC6 inhibitor is added to the cell culture within 6 or 12 hours of the transfection or transduction. In some embodiments, the selective HDAC6 inhibitor is added to the cell culture at least 1, 2, 3, 4, 5, 6 or 7 days before the transfection or transduction. In some embodiments, the selective HDAC6 inhibitor is added to the cell culture at least, or not more than, 1, 2, 3, 4, 5, 6, 7, 8, 9 or 10 days after the transfection or transduction.

In some embodiments, the selective HDAC6 inhibitor is added to the cell culture concurrently with the transfection or transduction.

In some embodiments, the cells are cultured for at least about 2, 3, 4, 5, 6, 7, 8, 9 or 10 days after the transfection or transduction. In some embodiments, the cells are cultured for about or at least about 3 days after the transfection or transduction.

In some embodiments, the cells are cultured for at least about 2, 3, 4, 5, 6, 7, 8, 9 or 10 days after both (i) the transfection or transduction, and (ii) the addition of the selective HDAC6 inhibitor. In some embodiments, the cells are cultured for about or at least about 3 days after both (i) the transfection or transduction, and (ii) the addition of the selective HDAC6 inhibitor.

In some embodiments, the cells are harvested after the culturing of the cells in the cell culture.

In some embodiments, the viral particles produced by the present methods are isolated or purified from the cells.

In some embodiments, the cells are transfected with: (i) one or more of the recombinant viral vector to be packaged in the cells into viral particles, and wherein the cells comprise or are further transfected with: (ii) one or more helper plasmids comprising a helper gene or genes; and/or (iii) one or more plasmids comprising one or more genes encoding a structural protein or proteins necessary for viral replication and/or encapsidation.

In some embodiments, the cells are transfected with: (i) one or more of the recombinant viral vector to be packaged in the cells into viral particles, wherein the viral vector is an AAV; and wherein the cells comprise or are further transfected with: (ii) one or more helper plasmids comprising a helper gene or genes; (iii) one or more plasmids comprising a rep gene and a cap gene. In some embodiments, the AAV is AAV9 or AAV5. In some embodiments, the cells are concurrently transfected with (i), (ii), and (iii). In some embodiments, the cells are transiently transfected with (i), (ii), and/or (iii). In some embodiments, the cells are stably transfected with (i), (ii), and/or (iii). In some embodiments, the cells are stably transfected with (ii) and/or (iii).

In some embodiments, the one or more helper plasmids expresses one or more of an adenovirus E1a gene, E1b gene, E2a gene, E4 gene and VA gene. In some embodiments, the one or more plasmids comprising a rep gene and a cap gene express a rep protein and a cap protein capable of packaging the recombinant viral vector.

In some embodiments, the recombinant viral vector used to transfect or transduce cells in a cell culture comprises a transgene. In some embodiments, the transgene expresses a therapeutic protein. In some embodiments, the recombinant viral vector encodes an siRNA or shRNA. In some embodiments, the recombinant viral vector encodes a guide RNA.

In some embodiments, the viral particle titer is increased at least or more than 20%, 25%, 30%, 40%, 50%, 60%, 70%, 80%, 90%, 100%, 200%, 300%, 400%, 500%, 1000% or 5000% compared to viral particle titer under the same conditions but in the absence of any viral sensitizer. In some embodiments, the viral particle titer is increased at least or more than 20%, 25%, 30%, 40%, 50%, 60%, 70%, 80%, 90%, 100%, 200%, 300%, 400%, 500%, 1000% or 5000% compared to viral particle titer under the same conditions but in the absence of the selective HDAC6 inhibitor.

In some embodiments, the method further comprises adding to the cell culture one or more additional compounds selected from the group consisting of a caspase inhibitor, a stimulator of interferon genes (STING) inhibitor, and a pan-HDAC inhibitor.

In some aspects, the present disclosure provides a method of producing viral particles and/or increasing viral particle titer, comprising: transfecting mammalian cells in a cell culture with a recombinant AAV9 vector to be packaged in the cells into viral particles; adding to the cell culture a fluoroalkyl-oxadiazole derivative (e.g. a compound of Formula (I), Formula (Ic), Formula (Ik), Formula (Ik-1), Formula (Ik-2), or Formula (Iv)); and culturing the cells in the cell culture.

In some aspects, the present disclosure provides a method of producing viral particles and/or increasing viral particle titer, comprising: transfecting mammalian cells in a cell culture with a recombinant AAV5 vector to be packaged in the cells into viral particles; adding to the cell culture a fluoroalkyl-oxadiazole derivative (e.g. a compound of Formula (I), Formula (Ic), Formula (Ik), Formula (Ik-1), Formula (Ik-2), or Formula (Iv)); and culturing the cells in the cell culture.

In some aspects, the fluoroalkyl-oxadiazole derivative is a compound of having the formula:

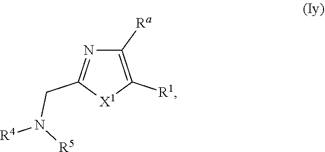

or a salt thereof,
wherein:
   X¹ is S;
   R^{a} is selected from the group consisting of H, halogen, and C₁₋₃ alkyl;
   R is

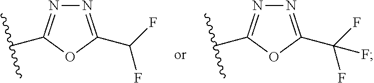 [SMILES: *c1nnc(C(F)F)o1 ; *c1nnc(C(F)(F)F)o1]

R² is selected from the group consisting of alkyl, alkoxy, and cycloalkyl, each of which is optionally substituted;
R³ is H or alkyl;
R⁴ is selected from the group consisting of alkyl, —(SO₂)R², —(SO₂)NR²R³, and —(CO)R²; and
R⁵ is aryl or heteroaryl; or R⁴ and R⁵ together with the atom to which they are attached form a heterocyclyl, each of which is optionally substituted.

In some embodiments, R^{a} is H.

In some embodiments, R¹ is

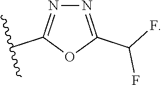 [SMILES: *c1nnc(C(F)F)o1]

In some embodiments, R⁴ is —(SO₂)R². In some embodiments, —(SO₂)R² is —(SO₂)alkyl, —(SO₂)alkyleneheterocyclyl, —(SO₂)haloalkyl, —(SO₂)haloalkoxy, or —(SO₂)cycloalkyl.

In some embodiments, R⁵ is heteroaryl. In some embodiments, the heteroaryl is a 5- to 6-membered heteroaryl. In some embodiments, the 5- to 6-membered heteroaryl is selected from the group consisting of

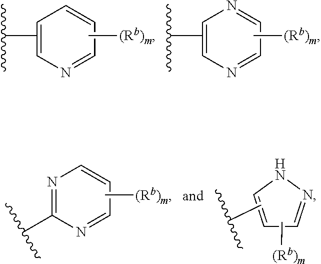

wherein R^{b} is halogen, alkyl, alkoxy, cycloalkyl, —CN, haloalkyl, or haloalkoxy; and m is 0 or 1. In some embodiments, R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments, R⁵ is aryl, and the aryl is selected from the group consisting of phenyl, 3-chlorophenyl, 3-chloro-4-fluorophenyl, 3-trifluoromethylphenyl, 3,4-difluorophenyl, and 2,6-difluorophenyl.

In some embodiments, the fluoroalkyl-oxadiazole derivative is:

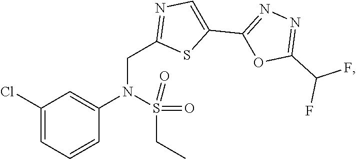 [SMILES: CCS(=O)(=O)N(Cc1ncc(-c2nnc(C(F)F)o2)s1)c1cccc(Cl)c1]

or an analog or salt thereof.

In some embodiments, the fluoroalkyl-oxadiazole derivative is:

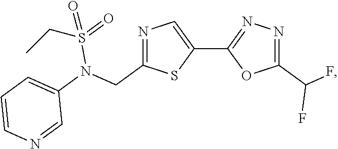 [SMILES: CCS(=O)(=O)N(Cc1ncc(-c2nnc(C(F)F)o2)s1)c1cccnc1]

an analog or salt thereof.

In some embodiments, the fluoroalkyl-oxadiazole derivative is:

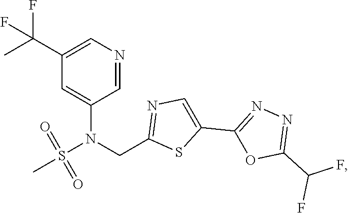 [SMILES: CC(F)(F)c1cncc(N(Cc2ncc(-c3nnc(C(F)F)o3)s2)S(C)(=O)=O)c1]

or an analog or salt thereof.

In some embodiments, the fluoroalkyl-oxadiazole derivative is:

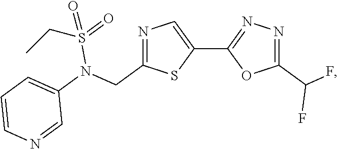 [SMILES: CCS(=O)(=O)N(Cc1ncc(-c2nnc(C(F)F)o2)s1)c1cccnc1]

or an analog or salt thereof.

In some embodiments, the fluoroalkyl-oxadiazole derivative is:

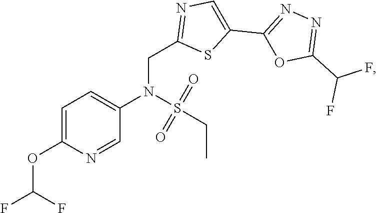 [SMILES: CCS(=O)(=O)N(Cc1ncc(-c2nnc(C(F)F)o2)s1)c1ccc(OC(F)F)nc1]

or an analog or salt thereof.

In some embodiments, the fluoroalkyl-oxadiazole derivative is:

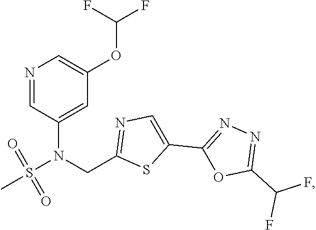 [SMILES: CS(=O)(=O)N(Cc1ncc(-c2nnc(C(F)F)o2)s1)c1cncc(OC(F)F)c1]

or an analog or salt thereof.

In some embodiments, the fluoroalkyl-oxadiazole derivative is:

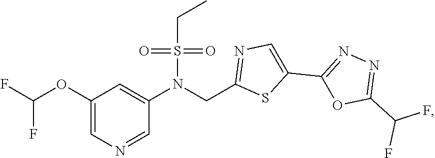 [SMILES: CCS(=O)(=O)N(Cc1ncc(-c2nnc(C(F)F)o2)s1)c1cncc(OC(F)F)c1]

or an analog or salt thereof.

In some embodiments, the fluoroalkyl-oxadiazole derivative is:

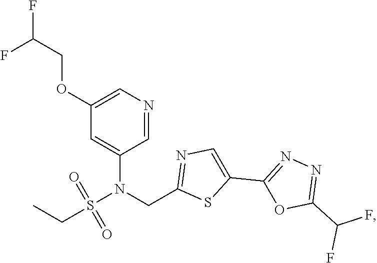 [SMILES: CCS(=O)(=O)N(Cc1ncc(-c2nnc(C(F)F)o2)s1)c1cncc(OCC(F)F)c1]

or an analog or salt thereof.

In some embodiments, the fluoroalkyl-oxadiazole derivative is:

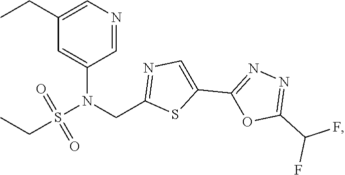 [SMILES: CCc1cncc(N(Cc2ncc(-c3nnc(C(F)F)o3)s2)S(=O)(=O)CC)c1]

or an analog or salt thereof.

In some embodiments, the fluoroalkyl-oxadiazole derivative is:

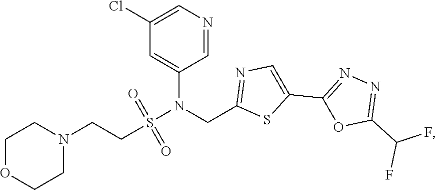 [SMILES: O=S(=O)(CCN1CCOCC1)N(Cc1ncc(-c2nnc(C(F)F)o2)s1)c1cncc(Cl)c1]

or an analog or salt thereof.

In some embodiments, the fluoroalkyl-oxadiazole derivative is:

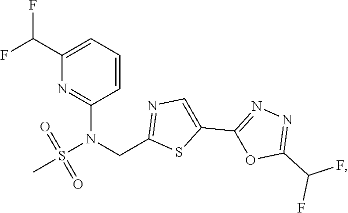 [SMILES: CS(=O)(=O)N(Cc1ncc(-c2nnc(C(F)F)o2)s1)c1cccc(C(F)F)n1]

or an analog or salt thereof.

In some embodiments, the fluoroalkyl-oxadiazole derivative is:

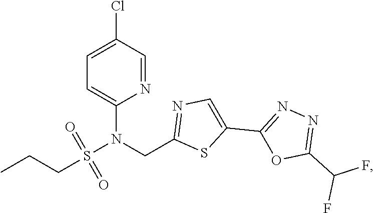 [SMILES: CCCS(=O)(=O)N(Cc1ncc(-c2nnc(C(F)F)o2)s1)c1ccc(Cl)cn1]

or an analog or salt thereof.

In some embodiments, the fluoroalkyl-oxadiazole derivative is:

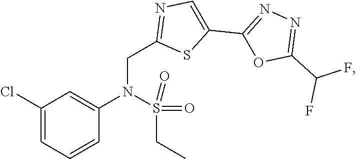 [SMILES: CCS(=O)(=O)N(Cc1ncc(-c2nnc(C(F)F)o2)s1)c1cccc(Cl)c1]

or an analog or salt thereof.

In some embodiments, the mammalian cells are HEK293 or HEK293T cells or cell line. In some embodiments, the HEK293 or HEK293T cells or cell line is/are of an early lineage. In some embodiments, the cells are ATCC 1573 HEK or another cell line of a similarly early lineage. In some embodiments, the early lineage is of 100 or less generations.

In some embodiments, the fluoroalkyl-oxadiazole derivative is added to the cell culture in an amount effective to achieve the concentration in the cell culture of between 0.1 μM and 100 μM, or between 0.2 μM and 50 μM. In some embodiments, the concentration of the fluoroalkyl-oxadiazole derivative in the cell culture is between about 0.5 μM and 15 μM. In some embodiments, the concentration of the fluoroalkyl-oxadiazole derivative in the cell culture is between about 1.25 μM and 5 μM.

In some embodiments, the transfected mammalian cells comprise or are further transfected with: one or more helper plasmids comprising a helper gene or genes, and/or one or more plasmids comprising a rep gene and a cap gene; optionally wherein the cells are concurrently or sequentially transfected with the recombinant AAV vector, the one or more helper plasmids, and/or the one or more plasmids comprising a rep gene and a cap gene. In some embodiments, the one or more helper plasmids expresses one or more of an adenovirus E1a gene, E1b gene, E2a gene, E4 gene and VA gene, and wherein the one or more plasmids comprising a rep gene and a cap gene express a rep protein and a cap protein capable of packaging the recombinant AAV. In some embodiments, the recombinant AAV comprises a transgene expressing a therapeutic protein, siRNA, shRNA, or guide RNA.

In some embodiments, the fluoroalkyl-oxadiazole derivative is added to the cell culture from 7 days before to 7 days after the transfection. In some embodiments, the fluoroalkyl-oxadiazole derivative is added to the cell culture from 2 days before to 2 days after the transfection or transduction. In some embodiments, the fluoroalkyl-oxadiazole derivative is added to the cell culture concurrently with the transfection or within 12 hours of the transfection. In some embodiments, the fluoroalkyl-oxadiazole derivative is added to the cell culture from 3 to 10 days before, or 7 to 10 days before, the transfection.

In some embodiments, the viral particle titer is increased at least or more than 20%, 25%, 30%, 40%, 50%, 60%, 70%, 80%, 90%, 100%, 200%, 300%, 400%, 500%, 1000% or 5000% compared to viral particle titer under the same conditions but in the absence of the fluoroalkyl-oxadiazole inhibitor.

In some embodiments of the present methods, the cells are harvested after the culturing of the cells in the cell culture, and the viral particles are isolated or purified from the cells.

In some embodiments, the present disclosure provides a method of producing viral particles and/or increasing viral particle titer, comprising: transfecting mammalian cells in a cell culture with a recombinant AAV9 vector to be packaged in the cells into viral particles; adding to the cell culture a fluoroalkyl-oxadiazole derivative (e.g. a compound of Formula (II) or Formula (III)); and culturing the cells in the cell culture.

In some embodiments, the cells are harvested after the culturing of the cells in the cell culture, and the viral particles are isolated or purified from the cells.

In some aspects, the present disclosure provides a method of producing viral particles and/or increasing viral particle titer, comprising: transfecting mammalian cells in a cell culture with a recombinant AAV5 vector to be packaged in the cells into viral particles; adding to the cell culture a hydroxamic acid derivative (e.g. a compound of Formula (II) or Formula (III)); and culturing the cells in the cell culture.

In some embodiments, the method further comprises adding to the cell culture one or more additional compounds selected from the group consisting of a caspase inhibitor, a stimulator of interferon genes (STING) inhibitor, and a pan-HDAC inhibitor.

### BRIEF DESCRIPTION OF DRAWINGS

FIG. 1 shows a schematic map of an exemplary rAAV vector encoding a transgene (human PKP2), comprising a cTnT promoter of 600 bp, the transgene, WPRE, bGH poly(a), and wherein this expression cassette is flanked by ITRs. This vector is termed “jy08-paav-1-2^{nd}-gen_ptnt600-hpkp2-optimized-wpre.” The PKP2 transgene was used in the experiments described in the examples.

FIG. 2 shows a schematic map of an exemplary helper plasmid, “pHelper”.

FIG. 3 shows a schematic map of an exemplary “pZC100_REP2_AAV9” rep/cap plasmid comprising a rep sequence and a wild-type AAV9 cap sequence.

FIG. 4 shows a schematic map of an exemplary “pZC107_REP2_AAV5” rep/cap plasmid comprising a rep sequence and a wild-type AAV5 cap sequence.

FIG. 5 shows a schematic map of an exemplary “pZC375_AAV9.VR-VIII-NSTYLG_VR-IV-KGSGQNQ” (“pZC375”) rep/cap plasmid comprising a rep sequence and a modified AAV9 cap sequence.

FIG. 6 shows a schematic map of an exemplary “pZC401_AAV9.VR-VIII-MMTTAR” (“pZC401”) rep/cap plasmid comprising a rep sequence and a modified AAV9 cap sequence.

FIG. 7 shows a schematic map of an exemplary “pZC428_AAV9.VR-VIII-NSTYLG” (“pZC428”) rep/cap plasmid comprising a rep sequence and a modified AAV9 cap sequence.

FIG. 8 shows a schematic map of an exemplary “pZC478_AAV9.VR-VIII-CSTSIR” (“pZC478”) rep/cap plasmid comprising a rep sequence and a modified AAV9 cap sequence.

FIG. 9 shows a schematic map of an exemplary “pZC96_REP2-CR9-10” (“pZC96”) rep/cap plasmid comprising a rep sequence and a modified AAV9 cap sequence.

FIGS. 10A-10B show AAV-GFP intensity screening (FIG. 10A) and mass spectrum analysis (FIG. 10B) (fold improvement over baseline) in HEK293 cell cultures to which tested compounds (including SMBs) were added. FIG. 10B shows an exemplary sample of liquid chromatography mass spectrometry (LC/MS) spectrum of the tested compound (SMB) at >99% purity. Abbreviation “SMBs” refers to small molecule boosters, which are selective HDAC6 inhibitors.

FIG. 11 shows that, when added to culture media, the tested compound improved scale-up productivity in 50 L and 200 L bioreactors (BRX). Successful scaling was achieved at both 50 L and 200 L BRX scale. Vg titer boosting by SMB was observed with AAV9 and other capsids (AAV9 variant capsids termed “novel capsid” 1, 2, 3 and 4) tested at BRX scale. “SMB” referenced in the figures from FIG. 11 to FIG. 17 is compound I-6 the structure of which is provided in Table 3. “SF” refers to shake flasks.

FIGS. 12A-12C show 3 L BRX scalability results with and without SMB. FIG. 12A shows viable cell density and % viable cell density comparisons of with and without SMB in cell culture. FIG. 12B shows that Vg titer with SMB increased by 39% compared to without SMB in 3 L BRX. FIG. 12C shows that metabolites were comparable with and without SMB in the shake flask satellites.

FIG. 13A shows Western blot images of cardiomyocytes transduced with affinity purified AAV:GFP from small scale shake flask productions (2 replicates—1 cultured with SMB and 1 without). Shows consistent ratios of VP1/VP2/VP3 with and without SMB. FIG. 13B shows GFP-transduced cardiomyocyte images and capillary electrophoresis (CE) electropherograms to verify SMB effects on AAV quality. The figure shows: (i) no significant impact of SMB addition on AAV infectivity, and (ii) from the CE electropherograms, VP1/VP2/VP3 ratios are found to be similar with and without SMB.

FIG. 14A shows purification recovery comparison, and FIG. 14B shows chromatograph of polishing step with and without SMB. SMB addition had no impact on purification performance.

FIG. 15 shows qPCR analysis of in vivo liver and heart samples from mice injected with AAV produced with and without SMB. No significant difference for in vivo transduction of liver and heart was observed between cultures with and without SMB.

FIG. 16 shows lentiviral titer with and without SMB. Lentiviral production with addition of SMB increased titer by more than 39% over a process without SMB addition.

FIG. 17 shows heatmap of enriched and depleted pathways in production culture with SMB relative to no SMB from RNAseq analysis.

FIG. 18 shows a schematic of the process for cell culturing in shake flasks for AAV production, with or without SMB addition, which may include a selective HDAC6 inhibitor and/or additional compounds.

FIG. 19 shows a schematic of the process for harvesting AAV from cell cultures for determining viral titer.

FIG. 20 shows the AAV viral titers of a DMSO control cell culture and cell cultures treated with TYA-004, a selective HDAC6 inhibitor, alone or in combination with other additional compounds. Viral titer was increased when the TYA-004 was added to the cell cultures. Viral titer was further boosted by addition of a combination of TYA-004, Ac-DEVD-CHO, and SN-011 (TYA-004+CHO+SN-011) and a combination of TYA-004, Ac-DEVD-CHO, SN-011, and Ricolinostat (TYA-004+CHO+SN-011+Ricolinostat).

### DETAILED DESCRIPTION

In some aspects, the present disclosure provides methods for producing viral particles comprising:
(i) introducing a viral particle or a viral vector into cells in a cell culture,
(ii) adding a selective HDAC6 inhibitor to the cell culture, and
(iii) culturing the cells in the cell culture.

In some aspects, the present disclosure provides methods for increasing viral particle titer comprising:
(i) introducing a viral particle or a viral vector into cells in a cell culture,
(ii) adding a selective HDAC6 inhibitor to the cell culture, and
(iii) culturing the cells in the cell culture.

In some embodiments, the cells are mammalian cells, such as HEK293 cells and HEK293T cells. Other mammalian cells, such as mammalian cells commonly used in viral production, can also be used in the methods described herein.

In some embodiments, the HDAC6 inhibitor (HDACi) is any selective HDAC6 inhibitor described herein or known in the art.

In some embodiments, a non-selective HDAC6 inhibitor is not used in the methods described herein. In some embodiments, a pan-HDAC inhibitor is not used in the methods described herein.

In some embodiments the virus is an adenovirus, an adeno-associated virus (AAV), a lentivirus, a retrovirus, a herpes virus, a herpes simplex virus, a vaccinia virus, an influenza virus, a rotavirus, a Hepatitis A virus, a CMV virus, an RSV virus, a rotavirus, or a rabies virus.

In some embodiments, the viral vector or viral particle is wild-type or natural. In some embodiments, the viral vector or viral particle is genetically modified. In some embodiment, the viral vector or viral particle is attenuated.

In some embodiments, a viral vector is introduced into cells already comprising genes encoding factors necessary for viral packaging. In some embodiments, a viral vector is introduced into cells together with one or more plasmids carrying genes encoding factors necessary for viral packaging.

In some embodiments, the addition of a selective HDAC6 inhibitor increases viral production in cells. In some embodiments, the addition of a selective HDAC6 inhibitor increases viral titer and yield.

In some embodiments, the use of the methods described herein achieves significantly better improvement in viral production, titer or yield than use of another viral sensitizer known in the art for the same purpose. In some embodiments, the use of the methods described herein achieves significantly better improvement in viral production, titer or yield than use of non-selective HDAC inhibitors or pan-HDAC inhibitors.

The present disclosure is based on the finding of unexpected efficacy of selective HDAC6 inhibitors, such as those described herein, in increasing viral production in a variety of mammalian cells.

### Terminology

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., time, concentration, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, about 1 to about 200 should be understood to include the explicitly recited limits of 1 and 200, but also to include sub-ranges such as about 10 to about 50, about 20 to about 100, and so forth. It also is to be understood, although not always explicitly stated, that the reagents described herein are merely exemplary and that equivalents of such are known in the art.

Also as used herein, “and/or” refers to and encompasses any and all possible combinations of one or more of the associated listed items, as well as the lack of combinations when interpreted in the alternative (“or”).

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

“AAV” is an abbreviation for adeno-associated virus. The term covers all subtypes of AAV, except where a subtype is indicated, and to both naturally occurring and recombinant forms. The abbreviation “rAAV” refers to recombinant adeno-associated virus. “AAV” includes AAV or any subtype. “AAV5” refers to AAV subtype 5. “AAV9” refers to AAV subtype 9. The genomic sequences of various serotypes of AAV, as well as the sequences of the native inverted terminal repeats (ITRs), Rep proteins, and capsid subunits may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), NC_001829 (AAV4), U89790 (AAV4), NC_006152 (AAV5), AF513851 (AAV7), AF513852 (AAV8), NC_006261 (AAV8), and AY530579 (AAV9). Publications describing AAV include Srivistava et al. (1983) J. Virol. 45:555; Chiorini et al. (1998) J. Virol. 71:6823; Chiorini et al. (1999) J. Virol. 73:1309; Bantel-Schaal et al. (1999) J. Virol. 73:939; Xiao et al. (1999) J Virol. 73:3994; Muramatsu et al. (1996) Virol. 221:208; Shade et al. (1986) J. Virol. 58:921; Gao et al. (2002) Proc. Nat. Acad. Sci. USA 99: 11854; Moris et al. (2004) Virology 33:375-383; Int′l Pat. Publ Nos. WO2018/222503A1, WO2012/145601A2, WO2000/028061A2, WO1999/61601A2, and WO1998/11244A2; U.S. patent application Ser. Nos. 15/782,980 and 15/433,322; and U.S. Pat. Nos. 10,036,016, 9,790,472, 9,737,618, 9,434,928, 9,233,131, 8,906,675, 7,790,449, 7,906,111, 7,718,424, 7,259,151, 7,198,951, 7,105,345, 6,962,815, 6,984,517, and 6,156,303.

An “AAV vector” or “rAAV vector” as used in the art to refer either to the DNA packaged into in the rAAV virion or to the rAAV virion itself, depending on context. As used herein, unless otherwise apparent from context, rAAV vector refers to a nucleic acid (typically a plasmid) comprising a polynucleotide sequence capable of being packaged into an rAAV virion, but with the capsid or other proteins of the rAAV virion. Generally an rAAV vector comprises a heterologous polynucleotide sequence (i.e., a polynucleotide not of AAV origin) and one or two AAV inverted terminal repeat sequences (ITRs) flanking the heterologous polynucleotide sequence. Only one of the two ITRs may be packaged into the rAAV and yet infectivity of the resulting rAAV virion may be maintained. See Wu et al. (2010) Mol Ther. 18:80. An rAAV vector may be designed to generate either single-stranded (ssAAV) or self-complementary (scAAV). See McCarty D. (2008) Mo. Ther. 16:1648-1656; WO2001/11034; WO2001/92551; WO2010/129021.

An “AAV particle” refers to an extracellular viral particle including at least one viral capsid protein (e.g. VP1) and an encapsidated AAV vector (or fragment thereof), including the capsid proteins.

For brevity and clarity, the disclosure refers to “capsid protein” or “capsid proteins” of AAV. Those skilled in the art understand that such references refer to VP1, VP2, or VP3, or combinations of VP1, VP2, and VP3. As in wild-type AAV and most recombinant expression systems VP1, VP2, and VP3 are expressed from the same open reading frame, engineering of the sequence that encodes VP3 inevitably alters the sequences of the C-terminal domain of VP1 and VP2. One may also express the capsid proteins from different open reading frames, in which case the capsid of the resulting rAAV virion could contain a mixture of wild-type and engineered capsid proteins, and mixtures of different engineered capsid proteins.

“Helper virus functions” refers to functions encoded in a helper virus genome which allow replication and packaging.

A “helper virus” for AAV refers to a virus that allows AAV (e.g. wild-type AAV) to be replicated and packaged by a mammalian cell. The helper viruses may be an adenovirus, herpesvirus or poxvirus, such as vaccinia.

“Packaging” refers to a series of intracellular events that result in the assembly of a viral particle, e.g., assembly of an rAAV viral particle or virion including encapsidation of the rAAV vector. AAV “rep” and “cap” genes refer to polynucleotide sequences encoding replication and encapsidation proteins of adeno-associated virus. AAV rep and cap are referred to herein as AAV “packaging genes.” Packaging requires either a helper virus itself or, more commonly in recombinant systems, helper virus function supplied by a helper-free system (i.e. one or more helper plasmids).

A viral particle or virion referenced herein is an infectious viral particle that comprises a competently assembled viral capsid and is capable of delivering a polynucleotide component into a cell for which the viral particle is tropic. In some embodiments, the viral particles described herein are replication-competent (i.e., capable of being replicated in an infected cell).

As used herein, the term “HDAC6” refers to the enzyme that in humans is encoded by the HDAC6 gene.

As used herein, the term “HDAC6 inhibitor” refers to a compound that inhibits at least one enzymatic activity of HDAC6.

An HDAC6 inhibitor may be a “selective” HDAC6 inhibitor. The term “selective” as used herein refers to selectivity against other HDACs, known in the art as “isozymes.” In some embodiments, the selectivity ratio of HDAC6 over HDAC1 is from about 5 to about 30,0000, e.g., about 5, about 10, about 20, about 30, about 40, about 50, about 60, about 70, about 80, about 90, about 100, about 1000, about 2000, about 3000, about 4000, about 5000, about 6000, about 7000, about 8000, about 9000, about 10,000, about 15,000, about 20,000, about 25,000, or about 30,000, including all values and ranges therebetween.

For example, a HDAC6 inhibitor may be at least 100-fold selective against HDAC6 compared to all other isozymes of HDAC. In some cases, selectivity may be determined by reference of another HDAC inhibitor, such as a pan-HDAC inhibitor—that is an inhibitor that inhibits HDACs other than HDAC6 in addition to HDAC6. Givinostat is an example of a pan-HDAC inhibitor. In some embodiments, a selective HDAC6 inhibitor inhibits HDACs other than HDAC6 at least 10-fold less effectively, at least 20-fold less effectively, at least 30-fold less effectively, at least 40-fold less effectively, at least 50-fold less effectively, at least 60-fold less effectively, at least 70-fold less effectively, at least 80-fold less effectively, at least 90-fold less effectively, or at least 100-fold less effectively than a pan-HDAC inhibitor, e.g., givinostat. In some embodiments, a selective HDAC6 inhibitor inhibits HDACs other than HDAC6 at least 100-fold less effectively than givinostat.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethynylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —OR^{a} where R^{a} is an alkyl, alkenyl or alknyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Carbocyclylalkyl” refers to a radical of the formula —R_{b}—R_{d} where R_{b} is an alkylene, alkenylene, or alkynylene group as defined above and R_{d} is a carbocyclyl radical as defined above. Unless stated otherwise specifically in the specification, a carbocyclylalkyl group can be optionally substituted.

“Cycloalkyl” refers to a stable non-aromatic monocyclic or polycyclic fully saturated hydrocarbon consisting solely of carbon and hydrogen atoms, which can include fused or bridged ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclycl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused or bridged ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Heterocyclylalkyl” refers to a radical of the formula —R_{b}—Rₑ where R_{b} is an alkylene, alkenylene, or alkynylene group as defined above and Rₑ is a heterocyclyl radical as defined above. Unless stated otherwise specifically in the specification, a heterocycloalkylalkyl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

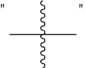

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

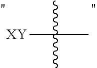

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

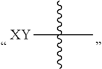

infers that when R³ is “XY”, the point of attachment bond is the same bond as the bond by which R³ is depicted as being bonded to CH₃.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “culture” or “cell culture” means the maintenance of cells in an artificial, in vitro environment. A “cell culture system” is used herein to refer to culture conditions in which a population of cells may be grown as monolayers or in suspension. “Culture medium” is used herein to refer to a nutrient solution for the culturing, growth, or proliferation of cells. Culture medium may be characterized by functional properties such as, but not limited to, the ability to maintain cells in a particular state (e.g., a pluripotent state, a quiescent state, etc.), to mature cells—in some instances, specifically, to promote the differentiation of progenitor cells into cells of a particular lineage (e.g., a cardiomyocyte).

The term “transfection” is as used herein refers to the uptake of an exogenous nucleic acid molecule by a cell. A cell has been “transfected” when exogenous nucleic acid has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acid molecules into suitable host cells.

The term “transduction” is as used herein refers to the transfer of an exogenous nucleic acid into a cell by a viral particle or virion.

The term “modified” refers to a substance or compound (e.g., a cell, a polynucleotide sequence, and/or a polypeptide sequence) that has been altered or changed as compared to the corresponding unmodified substance or compound.

As used herein, the term “transgene” refers to a nucleic acid sequence encoding a protein or RNA (e.g., a therapeutic protein), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

The term “expression cassette” refers to a polynucleotide cassette comprising a coding sequence which encodes a gene product of interest used to effect the expression of the gene product in target cells, which is operably linked to a promoter. Unless otherwise specified, the expression cassette of an AAV vector includes only the polynucleotides between (and not including) the ITRs.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

“Recombinant,” as applied to a polynucleotide means that the polynucleotide is the product of various combinations of cloning, restriction or ligation steps, and other procedures that result in a construct that is distinct from a polynucleotide found in nature, or that the polynucleotide is assembled from synthetic oligonucleotides. A “recombinant” protein is a protein produced from a recombinant polypeptide. A recombinant virion is a virion that comprises a recombinant polynucleotide and/or a recombinant protein, e.g. a recombinant capsid protein.

The term “isolated” means separated from constituents, cellular and otherwise, in which the virion, cell, tissue, polynucleotide, peptide, polypeptide, or protein is normally associated in nature. For example, an isolated cell is a cell that is separated form tissue or cells of dissimilar phenotype or genotype.

The term “purified” as used herein refers to material that has been isolated under conditions that reduce or eliminate the presence of unrelated materials, i.e. impurities, including native materials from which the material is obtained. For example, purified rAAV vector DNA is preferably substantially free of cell or culture components, including tissue culture components, contaminants, and the like.

The practice of the present disclosure will employ, unless otherwise indicated, conventional techniques of tissue culture, immunology, molecular biology, cell biology and recombinant DNA, which are within the skill of the art. See, e.g., Sambrook and Russell eds. (2001) Molecular Cloning: A Laboratory Manual, 3^{rd} edition; Ausubel et al. eds. (2007) Current Protocols in Molecular Biology; Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; IRL Press (1986) Immobilized Cells and Enzymes; Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (2002) Cold Spring Harbor Laboratory Press; Sohail (2004) Gene Silencing by RNA Interference: Technology and Application (CRC Press); and Sell (2013) Stem Cells Handbook.

Methods Of Improved Viral Production in Accordance with the Invention

The present disclosure provides selective histone deacetylase 6 inhibitors (HDAC6 inhibitors or HDACi) for use in improving viral production in a cell. In some embodiments, the present disclosure provides selective HDAC6i for use in increasing viral production in a mammalian cell (e.g., HEK293 or HEK293T). In some embodiments, the methods described herein increase viral titer. In some embodiments, the methods described herein increase viral yield. In some embodiments, the methods described herein increase viral production when used in at least or up to 3 liters (L), 50 L, or 200 L cell cultures (e.g., produced in a bioreactor). In some embodiments, the methods described herein increase viral production when used in at least or up to 1 kL cell cultures (e.g., produced in a bioreactor). In some embodiments, the methods descried herein achieve or maintain consistent viral yield in up to or at least 200 L cell cultures (e.g., in a bioreactor). In some embodiments, improvements in viral production using the methods described herein are production scale-independent. In some embodiments, improvements in viral production using the methods described herein are media-independent. In some embodiments, improvements in viral production using the methods described herein are cell or cell line-independent. In some embodiments, the methods described herein increase viral reproduction or replication. In some embodiments, the methods described herein increase viral packaging. In some embodiments, the methods described herein have no impact on viral product quality (e.g., as measured by consistent VP1/VP2/VP3 ratios in produced AAV). In some embodiments, the methods described herein have no impact on viral product purification recovery. In some embodiments, the methods described herein have no impact on viral product in vivo transduction efficiency (e.g., transduction of the heart and/or transduction of the liver).

In some embodiments, the methods described herein increase one or more of: (i) the transfection or transduction of cells with a viral vector or virus, (ii) the spread of the virus, and (iv) the expression of the transgene by the virus. In some embodiments, the methods described herein increase in vitro viral infectivity. In some embodiments, the methods described herein increase in vitro RNA and/or protein expression (e.g., of the transgene by the virus). In some embodiments, the methods described herein increase in vivo viral infectivity. In some embodiments, the methods described herein increase in vivo RNA and/or protein expression of the transgene by the virus.

In some embodiments, provided herein is a methods for producing viral particles comprising:
(i) introducing a viral vector into cells in a cell culture, optionally together with one or more plasmids carrying genes for packaging of the viral vector;
(ii) adding a selective HDAC6 inhibitor to the cell culture, which adding can be performed before, concurrently, or after step (i);
(iii) culturing the cells in the cell culture for a period of time, and
(iv) isolating and/or purifying viral particles comprising the packaged viral vector.

Exemplary cells suitable for use in the methods described herein are described below.

As described herein, in some embodiments, the cells are mammalian cells suitable for viral productions, such as HEK293 or HEK293T cells.

In some embodiments, the cells or cell lines described herein are maintained in cell culture medium (such as any cell culture medium described herein or known in the art). In some embodiments, the cells or cell lines described herein are maintained in serum (e.g., FBS) containing cell culture medium. In some embodiments, the cells or cell lines described herein are maintained in serum-free cell culture medium.

Exemplary viruses and viral vectors suitable for use in the methods described herein are described below. As described herein, in some embodiments, the virus to be produced is an AAV, a lentivirus or a retrovirus.

In some embodiments, the cells are transfected with one or more viral vectors carrying a transgene. The transgene can be any transgene, for example, a transgene carrying encoding a therapeutic protein. Alternatively, the viral vectors can be used for expression of therapeutic RNA molecules, such as siRNA, shRNA or guide RNA for selective targeting expression of a protein associated with a disease or disorder.

In some embodiments, the cells are further transfected with one or more helper plasmids or helper viruses (such as an adenovirus). In some embodiments, the cells are transfected with a plasmid that carries helper function genes required for viral production by a host cell (such as AAV replication). In some embodiments, the cells are further transfected with one or more vectors or plasmids comprising viral structural proteins (e.g., comprising viral structural proteins absent in the vector(s) carrying the transgene). In some embodiments, the cells are further transfected with one or more vectors or plasmids comprising viral structural proteins necessary for replication and/or encapsidation of the vector(s) carrying the transgene. In some embodiments, genetic material necessary for viral production is transiently transfected into a cell. In some embodiments, genetic material necessary for viral production is stably inserted into the cell genome. In some embodiments, the cell is a packaging cell, comprising genes necessary for viral production but not a vector genome carrying a transgene. In some embodiments, the cell is a producer cell, comprising genes necessary for viral production and a vector genome carrying a transgene.

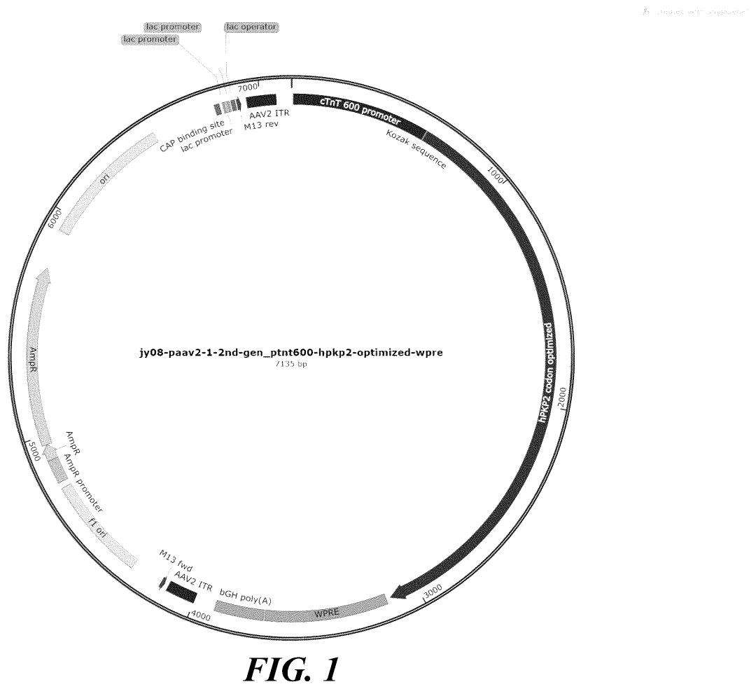

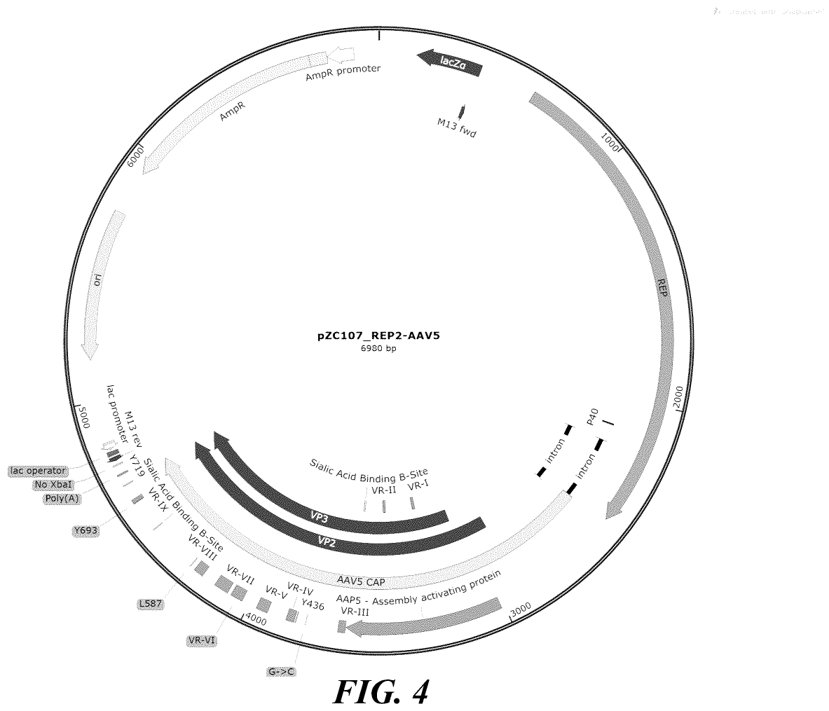

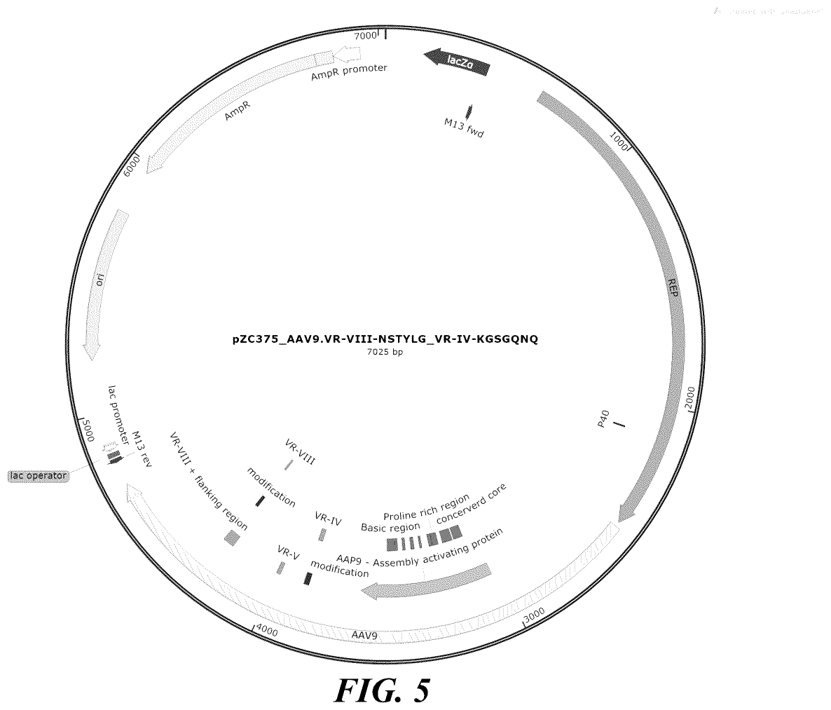

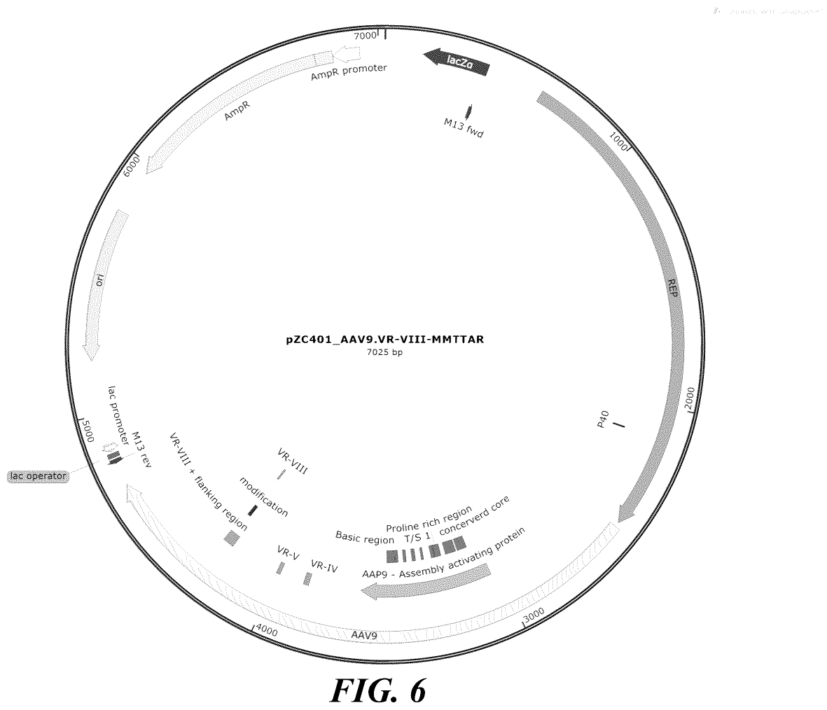

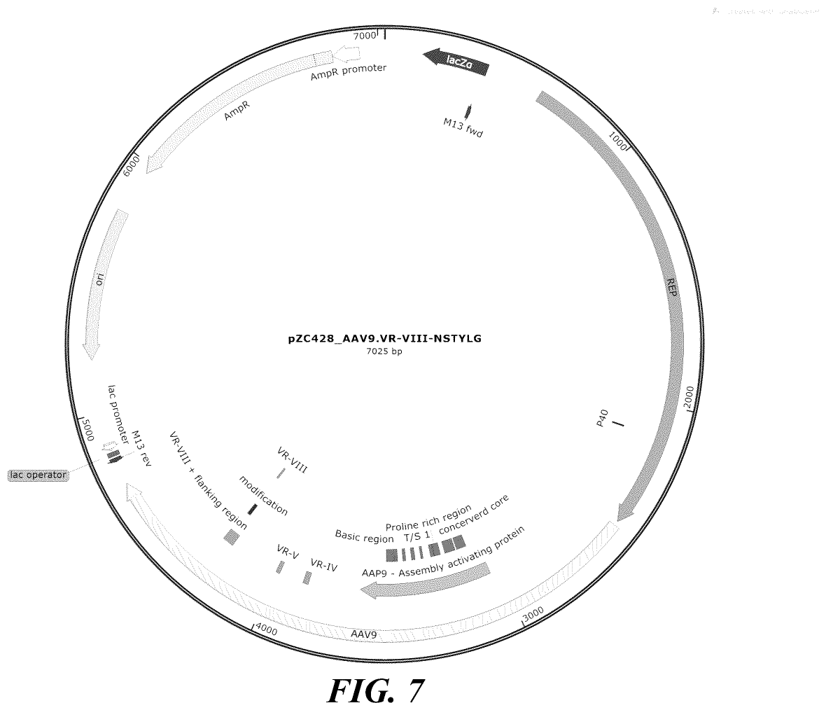

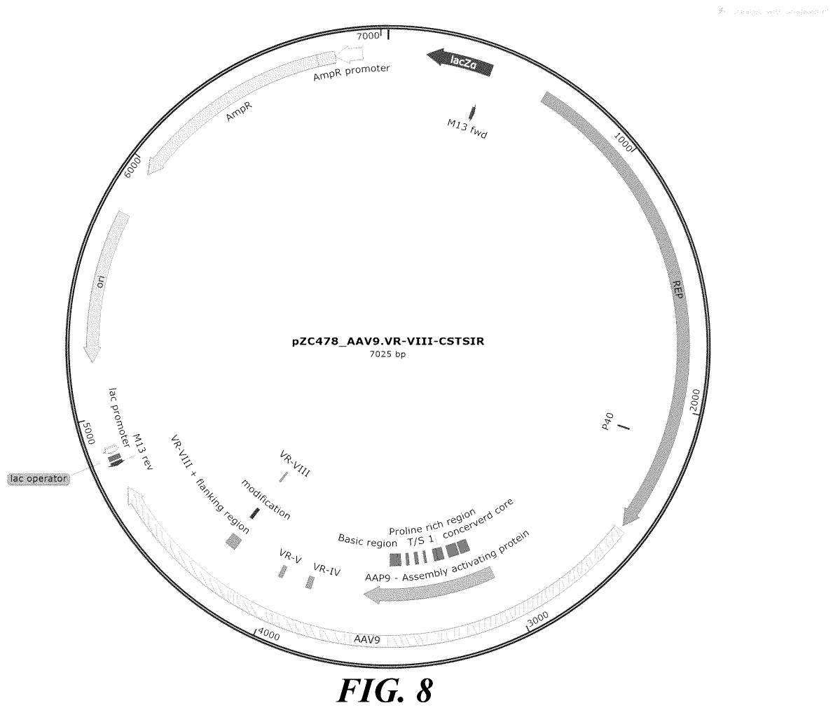

In some embodiments, the cells are transfected with an AAV vector carrying a transgene, a helper plasmid, and one or more vectors or plasmids carrying genes encoding a rep protein and a cap protein. An exemplary viral vector carrying a transgene, an exemplary helper plasmid, and exemplary vectors carrying genes encoding a rep protein and a cap protein are shown in FIGS. 1-8, and their sequences are shown in the Table 6.

In some embodiments, one or more nucleic acids encoding viral structural proteins necessary for replication and/or encapsidation are stably integrated into a cell, such as a producer cell for viral production. In some embodiments, such nucleic acids are chromosomally integrated into the cell. In some embodiments, one or more nucleic acids encoding viral structural proteins necessary for replication and/or encapsidation are transiently transfected into a cell, such as a producer cell for viral production.

In some embodiments, one or more helper function genes (such as those required for viral production by a host cell, e.g., AAV replication) are stably integrated into a cell, such as a producer cell for viral production. In some embodiments, such genes are chromosomally integrated into the cell. In some embodiments, one or more helper function genes (such as those required for viral production by a host cell, e.g., AAV replication) are transiently transfected into a cell, such as a producer cell for viral production.

In some embodiments, one or more transgenes (e.g., encoding a therapeutic gene product) are stably integrated into a cell, such as a producer cell for viral production. In some embodiments, such transgenes are chromosomally integrated into the cell. In some embodiments, one or more transgenes (e.g., encoding a therapeutic gene product) are transiently transfected into a cell, such as a producer cell for viral production.

In some embodiments, one, two, three or more of: a gene encoding a rep protein, a gene encoding a cap protein, a gene encoding helper functions and a transgene encoding a therapeutic gene product, are stably integrated into a cell, such as a producer cell for viral production. In some embodiments, such stably integrated genes are chromosomally integrated into the cell. In some embodiments, one, two, three or more of: a gene encoding a rep protein, a gene encoding a cap protein, a gene encoding helper functions and/or a transgene encoding a therapeutic gene product, are transiently transfected into a cell, such as a producer cell for viral production.

In some embodiments, gene encoding a rep protein, a gene encoding a cap protein, a gene encoding helper functions and a transgene encoding a therapeutic gene product are all transiently transfected into a cell.

In some embodiments, the cells are simultaneously transfected with the vectors/plasmids described hereinabove (such as a viral vector carrying a transgene, a helper plasmid and a vector/plasmid carrying structural proteins necessary for replication and/or encapsidation of the virus). In some embodiments, the cells are sequentially transfected with the vectors/plasmids described hereinabove (such as a viral vector carrying a transgene, a helper plasmid and a vector/plasmid carrying structural proteins necessary for replication and/or encapsidation of the virus).

In some embodiments, the cells already comprising (e.g., stably transfected with) rep, cap and/or helper function genes, are then further transfected with an AAV vector carrying a transgene.

In some embodiments, the cells are transfected with a vector or plasmid when cell viability reaches about or more than 90%. In some embodiments, the cells are transfected with a vector or plasmid when cell density at transfection is between about 0.5E+6 to 50E+6 cells/mL. In some embodiments, the cells are transfected with a vector or plasmid when cell density at transfection is between about 0.5E+6 to 25E+6 cells/mL. In some embodiments, the cells are transfected with a vector or plasmid when cell density at transfection is between about 1E+6 to 25E+6 cells/mL. In some embodiments, the cells are transfected with a vector or plasmid when cell density at transfection is between about 1E+6 to 10E+6 cells/mL. In some embodiments, the cells are transfected with a vector or plasmid when cell density at transfection is between about 1E+6 to 5E+6 cells/mL. In some embodiments, the cells are transfected with a vector or plasmid when cell density at transfection is between about 1.5E+6 to 3E+6 cells/mL. In some embodiments, the cells are transfected with a vector or plasmid when cell density at transfection of about 2E+6 cells/mL. In some embodiments, cell density at transfection is between about 0.5E+6 to 50E+6 cells/mL. In some embodiments, cell density at transfection is between about 0.5E+6 to 25E+6 cells/mL. In some embodiments, cell density at transfection is between about 1E+6 to 25E+6 cells/mL. In some embodiments, cell density at transfection is between about 1E+6 to 10E+6 cells/mL. In some embodiments, cell density at transfection is between about 1E+6 to 5E+6 cells/mL. In some embodiments, cell density at transfection is between about 1.5E+6 to 3E+6 cells/mL. In some embodiments cell density at transfection of about 2E+6 cells/mL. In some embodiments, the cell density referenced herein is a viable cell density.

Transfection of cells can be performed using any transfection methodology and reagent(s) known in the art or described herein. In some embodiments, transfection is chemical-based transfection. Suitable transfection reagents include but are not limited to calcium phosphate-based, cationic polymer-based, cationic lipid-based, and nonliposomal-based reagents. In some embodiments, transfection is physical transfection. Physical transfection methods include but are not limited to electroporation, sonoporation, and magnetic nanoparticle based methods.

In some embodiments, any suitable media can be used for cell culture described herein.

Suitable media is known in the art. In some embodiments serum-free media is used. In some embodiments, a chemically defined media is used. In some embodiments, media comprises serum. In some embodiments, media comprises fetal bovine serum (FBS). In some embodiments, the media is DMEM, MEM, or any media that is known to be used for mammalian cells, such as HEK293 cells. In some embodiments, the media comprises one or more buffers, salts, nutrients, vitamins, or other additives. In some embodiments, the media does not comprise any agents that are known to improve viral production (except for the selective HDAC6 inhibitors to be added). In some embodiments, the media is a commercially available medium, and does not comprise any additives.

In some embodiments, the cell are cultured in a suspension culture. In some embodiments, the cells are cultured in an attachment-dependent culture. In some embodiments, the cells are grown in a flask (e.g., spinner flask). In some embodiments, the cells are grown in a bioreactor.

Exemplary HDAC6 inhibitors for use in the methods described herein are described below. In some embodiments, an HDAC6 inhibitor is added to the cell culture in a solution.

In some embodiments, the HDAC6 inhibitors described herein are added to cell culture at the time the cells (to be used in viral production) are thawed. In some embodiments, the HDAC6 inhibitors described herein are added to cell culture during cell culture maintenance and/or expansion. In some embodiments, the HDAC6 inhibitors described herein are added to cell culture at the time of transfection with one or more plasmids or vectors. In some embodiments, the HDAC6 inhibitors described herein are added to cell culture simultaneously with one or more plasmids or vectors. In some embodiments, the HDAC6 inhibitors described herein are added to cell culture on the same day as the transfection with one or more plasmids or vectors. In some embodiments, the HDAC6 inhibitors described herein are added to cell culture sequentially with one or more plasmids or vectors (e.g., sequentially but on the same day, or sequentially and on different days).

In some embodiments, the HDAC6 inhibitors described herein are added to cell culture from 10 days before to 10 days after transfection with the viral vector carrying the transgene. In some embodiments, the HDAC6 inhibitors described herein are added to cell culture 10 or less days before (e.g., at most 10, 9, 8, 7, 6, 5, 4, 3, 2, or 1 days before) transfection with the viral vector carrying the transgene. In some embodiments, the HDAC6 inhibitors described herein are added to cell culture 10 days or less after (e.g., at most 1, 2, 3, 4, 5, 6, 7, 8, 9 or 10 days after) transfection with the viral vector carrying the transgene. In some embodiments, the HDAC6 inhibitors described herein are added to cell culture from about 10 days before (e.g., 10, 9, 8, 7, 6, 5, 4, 3, 2, or 1 days before) to about 10 days after (e.g., 10, 9, 8, 7, 6, 5, 4, 3, 2, or 1 days after) transfection with the vectors/plasmids described herein (such as the viral vector carrying a transgene, a helper plasmid or virus, and/or a vector or plasmid encoding structural proteins necessary for the viral replication and/or encapsidation) (and any time values in between these values).

In some embodiments, the HDAC6 inhibitors described herein are added to cell culture from about 7 days before to 7 days after transfection with the viral vector carrying the transgene. In some embodiments, the HDAC6 inhibitors described herein are added to cell culture from about 7 days before to 7 days after transfection with the vectors/plasmids described herein (such as the viral vector carrying a transgene, a helper plasmid or virus, and/or a vector or plasmid encoding structural proteins necessary for the viral replication and/or encapsidation).

In some embodiments, the HDAC6 inhibitors described herein are added to cell culture from about 5 days before to 5 days after transfection with the viral vector carrying the transgene. In some embodiments, the HDAC6 inhibitors described herein are added to cell culture from about 5 days before to 5 days after transfection with the vectors/plasmids described herein (such as the viral vector carrying a transgene, a helper plasmid or virus, and/or a vector or plasmid encoding structural proteins necessary for the viral replication and/or encapsidation).

In some embodiments, the HDAC6 inhibitors described herein are added to cell culture from about 3 days before to 3 days after transfection with the viral vector carrying the transgene. In some embodiments, the HDAC6 inhibitors described herein are added to cell culture from about 3 days before to 3 days after transfection with the vectors/plasmids described herein (such as the viral vector carrying a transgene, a helper plasmid or virus, and/or a vector or plasmid encoding structural proteins necessary for the viral replication and/or encapsidation).

In some embodiments, the HDAC6 inhibitors described herein are added to cell culture from about 1 day before to 1 day after transfection with the viral vector carrying the transgene. In some embodiments, the HDAC6 inhibitors described herein are added to cell culture from 1 day before to about 1 day after transfection with the vectors/plasmids described herein (such as the viral vector carrying a transgene, a helper plasmid or virus, and/or a vector or plasmid encoding structural proteins necessary for the viral replication and/or encapsidation).

In some embodiments, the HDAC6 inhibitors described herein are added to cell culture about the same time (e.g., within 3, 6, 12 or 18 hours) as transfection with the viral vector carrying the transgene. In some embodiments, the HDAC6 inhibitors described herein are added to cell culture about the same time (e.g., within 3, 6, 12 or 18 hours) as transfection with the vectors/plasmids described herein (such as the viral vector carrying a transgene, a helper plasmid or virus, and/or a vector or plasmid encoding structural proteins necessary for the viral replication and/or encapsidation).

In some embodiments, the HDAC6 inhibitors described herein are added to cell culture on the same day (e.g., within 6, 12 or 24 hours) as transfection with the vectors/plasmids described herein (such as the viral vector carrying a transgene, a helper plasmid or virus, and/or a vector or plasmid encoding structural proteins necessary for the viral replication and/or encapsidation). In some embodiments, the HDAC6 inhibitors described herein are added to cell culture on the same day (e.g., within 6, 12 or 24 hours) as transfection with the viral vector carrying the transgene. In some embodiments, the HDAC6 inhibitors described herein are added to cell culture simultaneously with transfection with the vectors/plasmids described herein (such as the viral vector carrying a transgene, a helper plasmid or virus, and/or a vector or plasmid encoding structural proteins necessary for the viral replication and/or encapsidation). In some embodiments, the HDAC6 inhibitors described herein are added to cell culture simultaneously with transfection with the viral vector carrying the transgene.

In some embodiments, the HDAC6 inhibitors described herein are added to cell culture from about 1 to 7 days before transfection with the viral vector carrying the transgene. In some embodiments, the HDAC6 inhibitors described herein are added to cell culture at least 1, 2, 3, 4, 5, 6 or 7 days before (e.g., at least 3 days before) transfection with the viral vector carrying the transgene. In some embodiments, the HDAC6 inhibitors described herein are added to cell culture from about 1 to 7 days before transfection with the vectors/plasmids described herein (such as the viral vector carrying a transgene, a helper plasmid or virus, and/or a vector or plasmid encoding structural proteins necessary for the viral replication and/or encapsidation). In some embodiments, the HDAC6 inhibitors described herein are added to cell culture at least 1, 2, 3, 4, 5, 6 or 7 days before (e.g., at least 3 days before) transfection with the vectors/plasmids described herein (such as the viral vector carrying a transgene, a helper plasmid or virus, and/or a vector or plasmid encoding structural proteins necessary for the viral replication and/or encapsidation).

In some embodiments, the HDAC6 inhibitors described herein are added to cell culture from about 5 to 10 days, or 7 to 10 days, before transfection with the viral vector carrying the transgene. In some embodiments, the HDAC6 inhibitors described herein are added to cell culture at least 5 days before transfection with the viral vector carrying the transgene. In some embodiments, the HDAC6 inhibitors described herein are added to cell culture at least 7 days before transfection with the viral vector carrying the transgene. In some embodiments, the HDAC6 inhibitors described herein are added to cell culture no more than 10 days before transfection with the viral vector carrying the transgene. In some embodiments, the HDAC6 inhibitors described herein are added to cell culture from about 5 to 10 days, or 7 to 10 days, before transfection with the vectors/plasmids described herein (such as the viral vector carrying a transgene, a helper plasmid or virus, and/or a vector or plasmid encoding structural proteins necessary for the viral replication and/or encapsidation). In some embodiments, the HDAC6 inhibitors described herein are added to cell culture at least 5 or 7 days before transfection with the vectors/plasmids described herein (such as the viral vector carrying a transgene, a helper plasmid or virus, and/or a vector or plasmid encoding structural proteins necessary for the viral replication and/or encapsidation). In some embodiments, the HDAC6 inhibitors described herein are added to cell culture no more than 10 days before transfection with the vectors/plasmids described herein (such as the viral vector carrying a transgene, a helper plasmid or virus, and/or a vector or plasmid encoding structural proteins necessary for the viral replication and/or encapsidation).

In some embodiments, the HDAC6 inhibitors described herein are added to cell culture from about 1 to 7 days after transfection with the viral vector carrying the transgene. In some embodiments, the HDAC6 inhibitors described herein are added to cell culture at least 1, 2, 3, 4, 5, 6 or 7 days after (e.g., at least 3 days after) transfection with the viral vector carrying the transgene. In some embodiments, the HDAC6 inhibitors described herein are added to cell culture from about 1 to 7 days after transfection with the vectors/plasmids described herein (such as the viral vector carrying a transgene, a helper plasmid or virus, and/or a vector or plasmid encoding structural proteins necessary for the viral replication and/or encapsidation). In some embodiments, the HDAC6 inhibitors described herein are added to cell culture at least 1, 2, 3, 4, 5, 6 or 7 days after (e.g., at least 3 days after) transfection with the vectors/plasmids described herein (such as the viral vector carrying a transgene, a helper plasmid or virus, and/or a vector or plasmid encoding structural proteins necessary for the viral replication and/or encapsidation).

In some embodiments, the cell culture is contacted with an HDAC6 inhibitor for at least or more than 1 day, 2 days, 3 days, 4 days, 5 days, 6 days or 7 days. In some embodiments, the cell culture is contacted with an HDAC6 inhibitor for at least or more than 2 or 3 days.

In some embodiments, the HDAC6 inhibitors described herein are used at concentrations between about 0.1 μM to 100 μM. In some embodiments, the HDAC6 inhibitors described herein are used at concentrations between about 0.2 μM to 75 μM. In some embodiments, the HDAC6 inhibitors described herein are used at concentrations between about 0.2 μM to 50 μM. In some embodiments, the HDAC6 inhibitors described herein are used at concentrations between about 0.5 μM to 25 μM. In some embodiments, the HDAC6 inhibitors described herein are used at concentrations between about 0.5 μM to 20 μM. In some embodiments, the HDAC6 inhibitors described herein are used at concentrations between about 0.5 μM to 10 μM. In some embodiments, the HDAC6 inhibitors described herein are used at concentrations between about 0.5 μM to 5 μM. In some embodiments, the HDAC6 inhibitors described herein are used at concentrations between about 0.75 μM to 15 μM. In some embodiments, the HDAC6 inhibitors described herein are used at concentrations between about 0.75 μM to 10 μM. In some embodiments, the HDAC6 inhibitors described herein are used at concentrations between about 0.75 μM to 5 μM. In some embodiments, the HDAC6 inhibitors described herein are used at concentrations between about 1 μM to 3 μM. In some embodiments, the HDAC6 inhibitors described herein are used at concentrations between about 1 μM to 2.5 μM. In some embodiments, the HDAC6 inhibitors described herein are used at concentrations between about 1.25 μM to 2.5 μM.

The concentrations of the HDAC6 inhibitors described herein are final concentrations in the cell culture medium after addition of the HDAC6 inhibitors described herein to the cell culture medium.

In some embodiments, the HDAC6 inhibitors described herein are used at a concentration of less than 500 μM. In some embodiments, the HDAC6 inhibitors described herein are used at a concentration of less than 250 μM. In some embodiments, the HDAC6 inhibitors described herein are used at a concentration of less than 200 μM. In some embodiments, the HDAC6 inhibitors described herein are used at a concentration of less than 150 μM. In some embodiments, the HDAC6 inhibitors described herein are used at a concentration of equal to or less than 100 μM. In some embodiments, the HDAC6 inhibitors described herein are used at a concentration of equal to or less than 75 μM. In some embodiments, the HDAC6 inhibitors described herein are used at a concentration of equal to or less than about 50 μM. In some embodiments, the HDAC6 inhibitors described herein are used at a concentration of equal to or less than about 25 μM. In some embodiments, the HDAC6 inhibitors described herein are used at a concentration of equal to or less than about 15 μM. In some embodiments, the HDAC6 inhibitors described herein are used at a concentration of equal to or less than about 10 μM.

In some embodiments, the HDAC6 inhibitors described herein are used at a concentration of about 0.75 μM. In some embodiments, the HDAC6 inhibitors described herein are used at a concentration of about 1 μM. In some embodiments, the HDAC6 inhibitors described herein are used at a concentration of about 1.25 μM. In some embodiments, the HDAC6 inhibitors described herein are used at a concentration of about 1.5 μM. In some embodiments, the HDAC6 inhibitors described herein are used at a concentration of about 2 μM. In some embodiments, the HDAC6 inhibitors described herein are used at a concentration of about 2.5 μM. In some embodiments, the HDAC6 inhibitors described herein are used at a concentration of about 3 μM. In some embodiments, the HDAC6 inhibitors described herein are used at a concentration of about 5 μM.

In some embodiments, the cells are harvested between 3 to 10 days post transfection for viral particle isolation and/or purification. In some embodiments, the cells are harvested at least or more than 3 days post transfection for viral particle isolation and/or purification. In some embodiments, the cells are harvested at least or more than 4 days post transfection for viral particle isolation and/or purification. In some embodiments, the cells are harvested at least or more than 5 days post transfection for viral particle isolation and/or purification. In some embodiments, the cells are harvested at least or more than 6 days post transfection for viral particle isolation and/or purification. In some embodiments, the cells are harvested at least or more than 7 days post transfection for viral particle isolation and/or purification. In some embodiments, the cells are harvested at least or more than 8 days post transfection for viral particle isolation and/or purification. In some embodiments, the cells are harvested 10 or less days post transfection for viral particle isolation and/or purification. In some embodiments, the cells are harvested 9 or less days post transfection for viral particle isolation and/or purification. In some embodiments, the cells are harvested 8 or less days post transfection for viral particle isolation and/or purification. In some embodiments, the cells are harvested 7 or less days post transfection for viral particle isolation and/or purification.

In some embodiments, HDAC6 inhibitors described herein can be diluted in 1 mM to 100 mM DMSO at stock concentrations of 0.25 mM to 1 mM. The stock solutions can then be added to the cell culture (e.g., to HEK293 or HEK293T cells) at 7 day before to 7 days after transfection at a working concentration between 0.1 μM to 100 μM. The cells can then be harvested between 3 days to 10 days post transfection for viral particle purification.

In some embodiments, the cells are treated to release viral particles. In some embodiments, the cells are harvested by lysis. Lysis can be performed by any chemical or enzymatic methods known in the art. For example, a protease, a nuclease, an endonuclease, a detergent, or a surfactant can be used for cell lysis. Alternatively, cell lysis can be performed by any physical disruption methods known in the art (e.g., sonication, freeze/thaw). In some embodiments, the virus is collected without cell lysis, e.g., by collecting secreted viral particles.

In some embodiments, viral particles are further purified from cell lysate or collected supernatant to remove cellular debris. Purification of viral particles can be performed using any method known in the art. Exemplary methods that can be used include, without limitation, affinity chromatography (such as Sepharose affinity resin), anion exchange chromatography, and density gradient purification (such as iodixanol-based and cesium-chloride based). Centrifugation and/or filtration methods can be used for viral particle purification.

Viral particle yield can be quantified using any method known in the art. For example, quantitative PCR, optical density or dot-blot hybridization can be used.

In some embodiments, the HDAC6 inhibitors described herein enhance viral yield or titer by at least, or more than, 20%, 25%, 30%, 35%, 40%, 45%, 50%, 55%, 60%, 65%, 70%, 75%, 80%, 90%, 95%, 100%, 125%, 150%, 175%, 200%, 250%, 300%, 350%, 400%, 450%, 500%, 600%, 700%, 800%, 900%, 1000%, 2000%, 3000%, 4000%, 5000%, 6000%, 7000%, 8000%, 9000%, 10000%, 11000%, 12000%, or 15000% (e.g., relative to the viral yield or titer under the same conditions but without the HDAC6 inhibitor described herein). In some embodiments, the HDAC6 inhibitors described herein enhance viral yield or titer by at least, or more than, 20%. In some embodiments, the HDAC6 inhibitors described herein enhance viral yield or titer by at least, or more than, 25%. In some embodiments, the HDAC6 inhibitors described herein enhance viral yield or titer by at least, or more than, 30%. In some embodiments, the HDAC6 inhibitors described herein enhance viral yield or titer by at least, or more than, 40%. In some embodiments, the HDAC6 inhibitors described herein enhance viral yield or titer by at least, or more than, 50%. In some embodiments, the HDAC6 inhibitors described herein enhance viral yield or titer by at least, or more than, 5000%. In some embodiments, the HDAC6 inhibitors described herein enhance viral yield or titer by at least, or more than, 9000%.

In some embodiments, the HDAC6 inhibitors described herein enhance AAV yield or titer by at least, or more than, 20%, 25%, 30%, 35%, 40%, 45%, 50%, 55%, 60%, 65%, 70%, 75%, 80%, 90%, 95%, 100%, 125%, 150%, 175%, 200%, 250%, 300%, 350%, 400%, 450%, 500%, 600%, 700%, 800%, 900%, 1000%, 2000%, 3000%, 4000%, 5000%, 6000%, 7000%, 8000%, 9000%, 10000%, 11000%, 12000%, or 15000% (e.g., relative to the viral yield or titer without the HDAC6 inhibitor described herein).

In some embodiments, the HDAC6 inhibitors described herein enhance lentiviral yield or titer by at least, or more than, 20%, 25%, 30%, 35%, 40%, 45%, 50%, 55%, 60%, 65%, 70%, 75%, 80%, 90%, 95%, 100%, 125%, 150%, 175%, 200%, 250%, 300%, 350%, 400%, 450%, 500%, 600%, 700%, 800%, 900%, 1000%, 2000%, 3000%, 4000%, 5000%, 6000%, 7000%, 8000%, 9000%, 10000%, 11000%, 12000%, or 15000% (e.g., relative to the viral yield or titer without the HDAC6 inhibitor described herein).

In some embodiments, the HDAC6 inhibitors described herein enhance retroviral yield or titer by at least, or more than, 20%, 25%, 30%, 35%, 40%, 45%, 50%, 55%, 60%, 65%, 70%, 75%, 80%, 90%, 95%, 100%, 125%, 150%, 175%, 200%, 250%, 300%, 350%, 400%, 450%, 500%, 600%, 700%, 800%, 900%, 1000%, 2000%, 3000%, 4000%, 5000%, 6000%, 7000%, 8000%, 9000%, 10000%, 11000%, 12000%, or 15000% (e.g., relative to the viral yield or titer without the HDAC6 inhibitor described herein).

In some embodiments, the HDAC6 inhibitors described herein enhance viral yield or titer (e.g., AAV, lentiviral or retroviral) by at least, or more than, 1.25-fold, 1.5-fold, 2-fold, 2.5-fold, 3-fold, 3.5-fold, 4-fold, 4.5-fold, 5-fold, 10-fold, 15-fold, 20-fold, 25-fold, 30-fold, 40-fold, 50-fold, 75-fold, or 100-fold (e.g., relative to the viral yield or titer under the same conditions but without the HDAC6 inhibitor described herein). In some embodiments, the HDAC6 inhibitors described herein enhance viral yield or titer (e.g., AAV, lentiviral or retroviral) by at least, or more than, 2-fold.

### Cells for Use in the Methods Described Herein

In some embodiments, the cells used in the methods described herein are mammalian cells.

In some embodiments, any mammalian cells or cell lines can be used in the methods described herein. In some embodiments, the mammalian cells or cell lines are any known in the art to be used for producing viral vectors or particles (e.g., AAV, lentivirus or retrovirus). In some embodiments, the mammalian cells or cell lines are any known in the art to be used as host cells for producing viral vectors or particles (e.g., AAV, lentivirus or retrovirus). In some embodiments, any commercial mammalian cell lines can be used in the methods described herein, e.g., any of those known to be suitable for use in producing viral vectors or particles.

In some embodiments, the cells used in the methods described herein are human cells. In some embodiments, the cells used in the methods described herein is a mammalian cell line. In some embodiments, the cells used in the methods described herein is a human cell line. In some embodiments, the cells used in the methods described herein are mammalian embryonic cells (e.g., human embryonic cells). In some embodiments, the cells used in the methods described herein is a mammalian embryonic cell line (e.g., a human embryonic cell line).

In some embodiments, the cells are primate cells. In some embodiments, the cells are mouse, dog, cat, rat, pig, or horse cells and the like.

In some embodiments, the cells used in the methods described herein are kidney cells. In some embodiments, the cells used in the methods described herein are human embryonic kidney cells.

In some embodiments, the cells are early lineage cells. In some embodiments, the cells are early lineage cells as assessed by the total number of doubling or generations. In some embodiments, the cells used in the methods described herein have undergone 100 or less doubling or generations.

In some embodiments, the cells or cell lines used in the methods described herein are selected from the group consisting of: are HEK293, HEK293T, HeLa, Vero, MDCK, MRC-5, PER.C6, BHK21 and CHO.

In some embodiments, the cells used in the methods described herein are HEK293 or HEK293T cells. In some embodiments, the cells used in the methods described herein are HEK293 cell line or HEK293T cell line. In some embodiments, the cells used in the methods described herein are HEK293 cells or cell line. In some embodiments, the cells used in the methods described herein are HEK293T cells or cell line. In some embodiments, any HEK293 or HEK293T cells described herein or known in the art can be used in the methods described herein.

In some embodiments, the cells used in the methods described herein are clonal derivatives of HEK293 cell. In some embodiments, the cells used in the methods described herein are HEK293F cells, HEK293T cells or HEK293-EXPI293 cells.

In some embodiments, the cells are “young” or early lineage HEK293 or HEK293T cells. In some embodiments, the HEK293 or HEK293T cells are at least or more than 100, 125, 150, 200, 250, 500, or 1000 generation younger than a commercial HEK293 cell line (e.g., Thermo Expi293 by ThermoFisher). In some embodiments, the HEK293 cells that have undergone 200 or less doubling or generations are used in the methods described herein. In some embodiments, the HEK293 cells that have undergone 100 or less doubling or generations are used in the methods described herein. In some embodiments, the HEK293T cells that have undergone 200 or less doubling or generations are used in the methods described herein. In some embodiments, the HEK293T cells that have undergone 100 or less doubling or generations are used in the methods described herein.

In some embodiments, the HEK293 or HEK293T cells are selected from the group consisting of: Expi-293 cells, Expi-239 cells, VPC 2.0 HEK293, ATCC 1573 HEK cells, and ATCC 3216 HEK293T.

In some embodiments, Expi-293 cells or cell lines are used in the methods described herein (such as any Expi-293 known in the art or described herein).

In some embodiments, Expi-239 cells or cell lines are used in the methods described herein (such as any Expi-239 known in the art or described herein).

In some embodiments, VPC 2.0 HEK293 cells or cell lines are used in the methods described herein (such as any VPC 2.0 HEK293 known in the art or described herein).

In some embodiments, ATCC 1573 HEK cells or cell lines are used in the methods described herein (such as any ATCC 1573 HEK known in the art or described herein).

In some embodiments, ATCC 3216 HEK293T cells or cell lines are used in the methods described herein (such as any ATCC 3216 HEK293T known in the art or described herein).

In some embodiments, the cells or cell lines used in the methods described herein are any of one of: HeLa, Vero, MDCK, MRC-5, PER.C6, BHK21 and CHO. In some embodiments, HeLa cells or cell line is used in the methods described herein. In some embodiments, Vero cells or cell line is used in the methods described herein. In some embodiments, MDCK cells or cell line is used in the methods described herein. In some embodiments, MRC-5 cells or cell line is used in the methods described herein. In some embodiments, PER.C6 cells or cell line is used in the methods described herein. In some embodiments, BHK21 cells or cell line is used in the methods described herein. In some embodiments, CHO cells or cell line is used in the methods described herein.

In some embodiments, the cells or cell lines described herein are maintained in cell culture medium (such as any cell culture medium described herein or known in the art). In some embodiments, the cells or cell lines described herein are maintained in serum containing cell culture medium. In some embodiments, the cells or cell lines described herein are maintained in serum-free cell culture medium.

### Viral Vectors and Particles for Use in the Methods Described Herein

In some embodiments, the viral vector or particle in the methods described herein is (or is derived from) an adenovirus, an adeno-associated virus (AAV), a lentivirus, a retrovirus, a herpes virus, a herpes simplex virus, a vaccinia virus, an influenza virus, a rotavirus, a Hepatitis A virus, a CMV virus, an RSV virus, a rabies virus, a parvovirus, a togavirus, a poxvirus, an alphavirus, a canarypox virus, a paramyoxovirus, a vesicular stomatitis virus, a baculovirus, a BacMam virus, or a Sendai virus.

In some embodiments, the viral vector or particle in the methods described herein is (or derived from) an adenovirus, an adeno-associated virus (AAV), a lentivirus, a retrovirus, a herpes virus, a herpes simplex virus, a vaccinia virus, an influenza virus, a rotavirus, a Hepatitis A virus, a CMV virus, an RSV virus, a vesicular stomatitis virus, or a rabies virus.

In some embodiments, the viral vector or particle is (or derived from) an AAV (e.g., a recombinant AAV, rAAV).

In some embodiments, the viral vector or particle is (or derived from) a lentivirus (e.g., a recombinant lentivirus).

In some embodiments, the viral vector or particle is (or derived from) a retrovirus (e.g., a recombinant retrovirus).

In some embodiments, the viral vector or particle is (or derived from) an adenovirus (e.g., a recombinant adenovirus).

In some embodiments, the viral vector or particle is (or derived from) a herpes virus (e.g., a recombinant herpes virus).

In some embodiments, the viral vector or particle is (or derived from) a herpes simplex virus (e.g., a recombinant herpes simplex virus).

In some embodiments, the viral vector or particle is (or derived from) a vaccinia virus (e.g., a recombinant vaccinia virus).

In some embodiments, the viral vector or particle is (or derived from) an influenza virus (e.g., a recombinant influenza virus).

In some embodiments, the viral vector or particle is (or derived from) a rotavirus (e.g., a recombinant rotavirus).

In some embodiments, the viral vector or particle is (or derived from) a Hepatitis A virus (e.g., a Hepatitis A virus).

In some embodiments, the viral vector or particle is (or derived from) a CMV virus (e.g., a recombinant CMV virus).

In some embodiments, the viral vector or particle is (or derived from) an RSV virus (e.g., a recombinant RSV virus).

In some embodiments, the viral vector or particle is (or derived from) a rabies virus (e.g., a recombinant rabies virus).

In some embodiments, the viral vector or particle is (or derived from) a vesicular stomatitis virus (e.g., a recombinant vesicular stomatitis virus).

In some embodiments, the viral vector or virus is natural.

In some embodiments, the viral vector or virus is attenuated.

In some embodiments, the viral vector or virus is genetically modified.

In some embodiments, the viral vectors described herein are replication-incompetent.

In some embodiments, the viral vectors described herein are replication-competent.

In some embodiments, the viral vectors described herein have no or low toxicity (i.e., have no effect on the physiology of the cell, such as a normal host cell).

In some embodiments, the viral vectors described herein are non-pathogenic and replication-defective.

In some embodiments, the viral vectors only contain viral sequences required for the initiation of viral DNA replication and packaging.

In some embodiments, the vectors are helper-dependent vectors (i.e., their production requires that the necessary structural proteins be provided to a cell in a separate vector or plasmid). In some embodiments, the viral vectors used in the methods described herein require a helper virus (e.g., replication-incompetent helper virus) or a helper plasmid (e.g., encoding a defective helper virus genome, see, e.g., those described in Saeki et al., 2001, Mol. Ther. 3:591-601).

In some embodiments, the vectors described herein are capable of being delivered to both dividing and non-dividing cells. In some embodiments, the vectors described herein are capable of being delivered to dividing cells. In some embodiments, the vectors described herein are capable of being delivered to non-dividing cells.

In some embodiments, any of the vectors described herein are recombinant vectors.

Any vector described herein can be transduced or introduced into cells.

In some embodiments, the vectors described herein comprise a transgene, e.g., as part of a transgene expression cassette. The transgene can be any transgene that is desired to be carried or expressed by the viral vector. In some embodiments, the vectors described herein further comprise one or more regulatory elements. In some embodiments, the vectors described herein comprise a promoter and/or enhancer operably linked to the transgene. In some embodiments the transgene expression cassette of the viral vector comprises, a transgene, a promoter, optionally an enhancer, and optionally one or more elements typically found in the virus used.

In some embodiments, the regulatory element is a non-translated region of the vector (e.g., origin of replication, selection cassettes, promoters, enhancers, translation initiation signals (Shine Dalgarno sequence or Kozak sequence) introns, a polyadenylation sequence, 5′ and 3′ untranslated regions) which interacts with host cellular proteins to carry out transcription and translation. Such elements may vary in their strength and specificity. In some embodiments, the regulatory element is one that is functional in a eukaryotic cell (e.g., a mammalian cell). In some embodiments, a polynucleotide sequence encoding a cardioprotective gene, gene product or inhibitory RNA described herein is operably linked to multiple control elements that allow expression of the polynucleotide in eukaryotic (e.g., mammalian) cells.

In some embodiments, the promoter is a tissue-specific promoter (e.g., a cardiac tissue-specific promoter, such as a TNNT2 promoter). In some embodiments, the promoter is a ubiquitous (strongly active in a wide range of cells) (e.g., the CAG promoter and CMB promoter, see Yue et al. BioTechniques 33:672-678 (2002). In some embodiments, the promoter is a constitutive (continually active) promoter. In some embodiments, the promoter is an inducible promoter. In some embodiments, the promoter is a cell-type specific promoter.

In some embodiments, the promoter is a cardiomyocyte-specific promoter. Examples of cardiac-specific or cardiomyocyte-specific promoter include, but are not limited to, a TNNT2 promoter, the alpha myosin heavy chain promoter, the myosin light chain 2v promoter, the alpha myosin heavy chain promoter, the alpha-cardiac actin promoter, the alpha-tropomyosin promoter, the cardiac troponin C promoter, the cardiac troponin I promoter, the cardiac myosin-binding protein C promoter, and the sarco/endoplasmic reticulum Ca²⁺ ATPase (SERCA) promoter (e.g. isoform 2 of SERCA2). Other promoters that can be used include but are not limited to: cytomegalovirus (CMV) immediate early, herpes simplex virus (HSV) thymidine kinase, a viral simian virus 40 (SV40) (e.g., early and late SV40), a spleen focus forming virus (SFFV) promoter, long terminal repeats (LTRs) from retrovirus (e.g., a Moloney murine leukemia virus (MoMLV) LTR promoter or a Rous sarcoma virus (RSV) LTR), a herpes simplex virus (HSV) (thymidine kinase) promoter, H5, P7.5, and P11 promoters from vaccinia virus, an elongation factor 1-alpha (EF1α) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4A1 (EIF4A1) promoter, a heat shock 70 kDa protein 5 (HSPA5) promoter, a heat shock protein 90 kDa beta, member 1 (HSP90B1) promoter, a heat shock protein 70 kDa (HSP70) promoter, a 0-kinesin (O—KIN) promoter, the human ROSA 26 locus (Irions et al., Nature Biotechnology 25, 1477-1482 (2007)), a Ubiquitin C (UBC) promoter, a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer-binding site substituted (MND) promoter, and mouse metallothionein-1.

In some embodiments, the enhancer is a tissue-specific enhancer (e.g., a cardiac tissue-specific enhancer). The enhancer can be operably linked to a promoter and modulate the expression of a transgene operably linked to a promoter. In some embodiments, the enhancer comprises an ACTC1 cardiac enhancer (ACTC1e). In some embodiments, the enhancer comprises an αMHC enhancer (αMHCe).

In some embodiments, the vector described herein is an AAV. In some embodiments, the AAV contains any one or more of the elements typically found in AAV, e.g., a polyadenylation sequence (e.g., a BGH poly(A) sequence, or a SV40 poly(A) sequence), an intron (e.g., a CMV intron or a chimeric intron), a post-translational regulatory element (e.g., a Woodchuck Post-transcriptional Regulatory Element (WPRE) or a modified WPRE), and/or a transcription termination signal. In some embodiments, the AAV vector genome containing a transgene expression cassette is flanked by inverted terminal repeats (such as a 5′ ITR and a 3′ ITR). In some embodiments, the AAV vector requires helper factors to replicate. The helper factors can be provided by coinfecting a helper virus (e.g., adenovirus, herpesvirus or papillomavirus) or helper plasmid. Exemplary helper viruses and plasmids are described herein. Any helper virus or plasmid known in the art can be used in the methods described herein (see, e.g., Meier et al., 202, Viruses 12(6):662). In some embodiments, the helper genes can be any one or more of adenoviral Ela, E1b, E2a, E4 and VA genes. In some embodiments, a helper plasmid comprises adenovirus E2a and E4 genes, and optionally VA RNA.

In some embodiments, the viral vector is an AAV vector of any serotype. In some embodiments, the viral vector is AAV selected from the group consisting of serotype 1, 2, 3, 4, 5, 6, 7, 8, 9, 10, 11, 12, rh.10, rh.20, rh.74, and a chimeric AAV derived thereof. In some embodiments, the AAV is AAV1, AAV2, AAV3, AAV4, AAV5, AAV6, AAV7, AAV8, AAV9, AAV10, AAV11, AAV12, AAVrh.10, AAVrh.20, AAVrh.74, or a variant thereof.

In some embodiments, the AAV is AAV9 or a variant thereof.

In some embodiments, the AAV is AAV5 or a variant thereof.

The rAAV virions or particles of the disclosure comprise a capsid protein. Capsid proteins are structural proteins that make up the assembled icosahedral packaging of the rAAV virion that contains the expression cassette. Capsid proteins are classified by the serotype. Wild-type capsid serotypes in rAAV virions can be, for example, AAV1, AAV2, AAV3, AAV4, AAV5, AAV6, AAV7, AAV8, AAV9, AAV10, AAV11, AAV12, AAVrh.10, AAVrh.20, or AAVrh.74 (Naso et al. BioDrugs 31:317-334 (2017)). Engineered capsid types include chimeric capsids and mosaic capsids (Choi et al. Curr Gene Ther. 5: 299-310 (2005)). Capsids are selected for rAAV virions based on their ability to transduce specific tissue or cell types (Liu et al. Curr Pharm Des. 21:3248-56 (2015)).

Any capsid protein that can facilitate rAAV virion transduction into cardiac cells for delivery of a transgene, as described herein, can be used. Capsid proteins used in rAAV virions for transgene delivery to cardiac cells that result in high expression can be, for example, AAV4, AAV6, AAV7, AAV8, and AAV9 (Zincarelli et al. Mol. Ther. 16:P1073-1080 (2008)). Artificial capsids, such as chimeric capsids generated through combinatorial libraries, can also be used for transgene delivery to cardiac cells that results in high expression (see U.S. 63/012,703, the contents of which are herein incorporated by reference). Other capsid proteins with various features can also be used in the rAAV virions of the disclosure. AAV vectors and capsids are provided in U.S. Pat. Pub. Nos. U.S. Ser. No. 10/011,640B2; U.S. Pat. No. 7,892,809B2, U.S. Pat. No. 8,632,764B2, U.S. Pat. No. 8,889,641B2, U.S. Pat. No. 9,475,845B2, U.S. Ser. No. 10/889,833B2, U.S. Ser. No. 10/480,011B2, and U.S. Ser. No. 10/894,949B2, the contents of which are herein incorporated by reference; and Int′l Pat. Pub. Nos. WO2020198737A1, WO2019028306A2, WO2016054554A1, WO2018152333A1, WO2017106236A1, WO2008124724A1, WO2017212019A1, WO2020117898A1, WO2017192750A1, WO2020191300A1, and WO2017100671A1, the contents of which are herein incorporated by reference. In some embodiments, the rAAV virions of the disclosure comprise an engineered capsid protein. Engineered capsid proteins can be derived from a parental, e.g. wild-type, capsid and include, for example, variant polypeptide sequence with respect to a parental capsid sequence at one or more sites. For example, variant sites of the parental capsid can occur at the VR-IV site, VR-V site, VR-VII site and/or VR-VIII site (see, e.g., Buning and Srivastava. Mol Ther Methods Cin Dev. 12:248-265 (2019)).

In some embodiments, the capsid protein is an AAV5/AAV9 chimeric capsid protein. In some embodiments, at least one polypeptide segment is derived from the AAV5 capsid protein and at least one polypeptide segment is derived from the AAV9 capsid protein. In some embodiments, the one or more sites of the chimeric parental sequence are selected from those equivalent to the VR-IV site, the VR-V site, the VR-VII site and the VR-VIII site of the AAV9 capsid protein.

In some embodiments, a mutant capsid protein is used in the methods described herein, such as any mutant described herein or known in the art. In some embodiments, a mutant capsid protein, which is associated with or known to lead to a lower viral yield than the corresponding wild-type capsid protein, is used in the methods described herein. In some embodiments, a mutant capsid protein, wherein the mutation is associated with or known to lead to a lower viral yield relative to a capsid protein without the mutation, is used in the methods described herein.

In some embodiments, a mutant AAV (e.g., AAV9) capsid protein is used in the methods described herein, such as any mutant described herein or known in the art. In some embodiments, a mutant AAV (e.g., AAV9) capsid protein, which is associated with or known to lead to a lower viral yield than the corresponding wild-type capsid protein, is used in the methods described herein. In some embodiments, a mutant AAV (e.g., AAV9) capsid protein, wherein the mutation is associated with or known to lead to a lower viral yield relative to a capsid protein without the mutation, is used in the methods described herein.

In some embodiments, the vector described herein is a lentiviral vector (LV). In some embodiments, the LV contains any one or more of the elements typically found in lentiviral vectors, e.g., a vector genome packaging signal, a Rev Responsive Element (RRE), a polypurine tract (e.g., a central polypurine tract, a 3′ polypurine tract, etc.), and/or a post-translational regulatory element (e.g., a Woodchuck Post-transcriptional Regulatory Element (WPRE) or a modified WPRE). In some embodiments, the LV vector genome containing a transgene expression cassette is flanked by cis-acting long terminal repeats. In some embodiments, the LV is self-inactivating (SIN). In some embodiments, the LV described herein is TAT-independent.

### AAV Viral Capsid Proteins for Use in the Methods

In some embodiments, the viral vector or particle in the methods described herein is (or is derived from) an AAV involving recombinant, engineered, or otherwise modified adeno-capsid proteins. Capsids are selected for rAAV virions based on their ability to transduce specific tissue or cell types. In some embodiments, an AAV capsid protein referenced herein is any AAV capsid protein of any serotype known in the art or described herein, or a variant thereof. In some embodiments, the AAV serotype is selected from the group consisting of serotypes 1, 2, 3, 4, 5, 6, 7, 8, 9, 10, 11, 12, rh.10, rh.20, rh.74, and a chimeric AAV derived therefrom. In some embodiments, the AAV is AAV1, AAV2, AAV3, AAV4, AAV5, AAV6, AAV7, AAV8, AAV9, AAV10, AAV11, AAV12, AAVrh.10, AAVrh.20, AAVrh.74, or a variant thereof.

The wild-type AAV9 VP1 has the amino acid sequence of SEQ ID NO: 11. The wild-type AAV9 VP2 has the amino acid sequence of SEQ ID NO: 12. The wild-type AAV9 VP3 has the amino acid sequence of SEQ ID NO: 13. In some embodiments, the capsid protein described herein comprises a sequence that shares at least about 80%, 85%, 90%, 95%, 96%, 97%, 98%, 99%, 99.5%, or 100% identity to SEQ ID NO: 11, as shown below. In some embodiments, the capsid protein described herein comprises a sequence that shares at least about 80%, 85%, 90%, 95%, 96%, 97%, 98%, 99%, 99.5%, or 100% identity to SEQ ID NO: 12. In some embodiments, the capsid protein described herein comprises a sequence that shares at least about 80%, 85%, 90%, 95%, 96%, 97%, 98%, 99%, 99.5%, or 100% identity to SEQ ID NO: 13. The N-terminal residue of VP1, VP2, and VP3, as well as the VR sites (VR-1, VR-11, VR-IV, VR-V, VR-VII and VR-VIII), are indicated (in bold, and underlined) in the sequence of full-length VP1 (SEQ ID NO: 11) below.

```
(SEQ ID NO: 11)
VP1-->
MAADGYLPDWLEDNLSEGIREWWALKPGAPQPKANQQHODNARGLVLPGYKYLGPGNGLDKG
EPVNAADAAALEHDKAYDQQLKAGDNPYLKYNHADAEFQERLKEDTSFGGNLGRAVFQAKKR
(SEQ ID NO: 12)
                          VP2-->
LLEPLGLVEEAAKTAPGKKRPVEQSPQEPDSSAGIGKSGAQPAKKRLNFGQTGDTESVPDPQ
(SEQ ID NO: 13)
                                VP3-->
PIGEPPAAPSGVGSLTMASGGGAPVADNNEGADGVGSSSGNWHCDSQWLGDRVITTSTRTWA
LPTYNNHLYKQISNSTSGGSSNDNAYFGYSTPWGYFDENRFHCHESPRDWORLINNNWGFRP
KRLNFKLFNIQVKEVTDNNGVKTIANNLTSTVQVFTDSDYQLPYVLGSAHEGCLPPFPADVE
MIPQYGYLTLNDGSQAVGRSSFYCLEYFPSQMLRTGNNFQFSYEFENVPFHSSYAHSQSLDR
                                  VR-IV
LMNPLIDQYLYYLSKTINGSGQNQQTLKFSVAGPSNMAVOGRNYIPGPSYRQQRVSTTVTON
VR-V VR-VII
NNSEFAWPGASSWALNGRNSLMNPGPAMASHKEGEDRFFPLSGSLIFGKQGTGRDNVDADKV
                                            VR-VIII
MITNEEEIKTTNPVATESYGQVATNHQSAQAQAQTGWVQNQGILPGMVWQDRDVYLQGPIWA
KIPHTDGNFHPSPLMGGFGMKHPPPQILIKNTPVPADPPTAFNKDKLNSFITQYSTGQVSVE
IEWELQKENSKRWNPEIQYTSNYYKSNNVEFAVNTEGVYSEPRPIGTRYLTRNL
```

As labeled in AAV9 VP1 (SEQ ID NO: 11) above, the VR-IV site is between amino acids 452 and 458 in the parental sequence (“NGSGQNQ”, SEQ ID NO: 15); the VR-V site is between amino acids 497 and 502 in the parental sequence (“NNSEFA”, SEQ ID NO: 16); the VR-VII site is between amino acids 549 and 553 in the parental sequence (“GRDNV”, SEQ ID NO: 17); the VR-VIII site is between amino acids 581 and 594 in the parental sequence (“ATNHQSAQAQAQTG”, SEQ ID NO: 18). In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 11, excluding the VR-IV, VR-V, VR-VII, and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 11, excluding the VR-IV and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 11, excluding the VR-IV site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 11, excluding the VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 12, excluding the VR-IV, VR-V, VR-VII, and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 12, excluding the VR-IV and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 12, excluding the VR-IV site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 12, excluding the VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 13, excluding the VR-IV, VR-V, VR-VII, and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 13, excluding the VR-IV and/or VR-VIII site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 13, excluding the VR-IV site. In some embodiments, the capsid protein comprises a sequence that shares at least about 80% (e.g., at least 80%, 85%, 90%, 95%, 96%, 97%, 98%, 99%, 99.5%, or 100%) identity to SEQ ID NO: 13, excluding the VR-VIII site.

In some embodiments, the capsid protein is any capsid protein described herein or a variant thereof, e.g., an AAV9 variant capsid protein (e.g., comprising one or more substitutions or insertions) in the wild-type AAV9 capsid protein sequence described herein.

In some embodiments, the capsid protein comprises one, two, three, four or more substitutions in the VR-VIII site. In some embodiments, the capsid protein comprises one, two, three, four or more insertions in the VR-VIII site. In some embodiments, the capsid protein comprises, relative to reference SEQ ID NO: 11, one, two, three, four or more substitutions at positions from 584 to 590 in the VR-VIII site, or one, two, three, four or more substitutions at positions from 585 to 590 in the VR-VIII site. In some embodiments, the capsid protein comprises, relative to reference SEQ ID NO: 11, one, two, three, four or more insertions at positions from 584 to 590 in the VR-VIII site, or one, two, three, four or more insertions at positions from 585 to 590 in the VR-VIII site.

In some embodiments, the capsid protein comprises at least two, three, four, five or more substitutions in the VR-VIII site. In some embodiments, the capsid protein comprises at least two, three, four or more insertions in the VR-VIII site. In some embodiments, the capsid protein comprises, relative to reference SEQ ID NO: 11, at least two, three, four, five or more substitutions at positions from 584 to 590 in the VR-VIII site, or at least two, three, four, five or more substitutions at positions from 585 to 590 in the VR-VIII site. In some embodiments, the capsid protein comprises, relative to reference SEQ ID NO: 11, at least two, three, four or more insertions at positions from 584 to 590 in the VR-VIII site, or at least two, three, four or more insertions at positions from 585 to 590 in the VR-VIII site.

In some embodiments, the capsid protein may comprise an amino acid insertion at position 584 (relative to reference sequence SEQ ID NO: 11) comprising one or more of an asparagine (N), a threonine (T), a tyrosine (Y), phenylalanine (F), and an alanine (A).

In some embodiments, the capsid protein may comprise an amino acid insertion at position 585 (relative to reference sequence SEQ ID NO: 11) comprising one or more of a histidine (H) and a methionine (M).

In some embodiments, the capsid protein may comprise an amino acid insertion at position 586 (relative to reference sequence SEQ ID NO: 11) comprising one or more of a histidine (H), a tyrosine (Y), a valine (V), a threonine (T), an alanine (A), an isoleucine (I), a tryptophan (W), a methionine (M), and a leucine.

In some embodiments, the capsid protein may comprise an amino acid insertion at position 587 (relative to reference sequence SEQ ID NO: 11) comprising one or more of an isoleucine (I) and a proline (P).

In some embodiments, the capsid protein may comprise an amino acid insertion at position 588 (relative to reference sequence SEQ ID NO: 11) comprising one or more of an isoleucine (I), a threonine (T), and a proline (P).

In some embodiments, the capsid protein may comprise one or more amino acid substitutions selected from the group consisting of N452K, N452A, N452V, G453A, G453N, S454T, S454D, G455N, Q456L, Q456K, N457L, N457V, Q458I, and Q458H (relative to reference sequence SEQ ID NO: 11).

In some embodiments, the capsid protein may comprise one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A5911I, G594Q, and G594D (relative to reference sequence SEQ ID NO: 11).

In some embodiments, the capsid protein may comprise an amino acid insertion at position 584 (relative to reference sequence SEQ ID NO: 11) consisting of a TY, FN, or AT.

In some embodiments, the capsid protein may comprise an amino acid insertion at position 585 (relative to reference sequence SEQ ID NO: 11) consisting of MH.

In some embodiments, the capsid protein may comprise an amino acid insertion at position 586 (relative to reference sequence SEQ ID NO: 11) consisting of HY, VT, AI, WM, or ML.

In some embodiments, the capsid protein may comprise an amino acid insertion at position 587 (relative to reference sequence SEQ ID NO: 11) consisting of PI.

In some embodiments, the capsid protein may comprise an amino acid insertion at position 588 (relative to reference sequence SEQ ID NO: 11) consisting of IT or PT.

In some embodiments, the capsid protein may comprise one or more amino acid substitutions selected from the group consisting of T582D, T582E, N583V, H584Q, S586K, A587P, A587S, Q588G, Q588M, A589S, A591I, G594Q, and G594D (relative to reference sequence SEQ ID NO: 11).

In some embodiments, the capsid protein may comprise one or more amino acid substitutions selected from the group consisting of T582L, T582A, T582F, T582R, T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588T, and Q588R (relative to reference sequence SEQ ID NO: 11).

In some embodiments, the capsid protein may comprise one or more amino acid substitutions selected from the group consisting of Q585C, Q585S, and S586I (relative to reference sequence SEQ ID NO: 11).

In some embodiments, the capsid protein may comprise one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L (relative to reference sequence SEQ ID NO: 11).

In some embodiments, the capsid protein may comprise one or more amino acid substitutions selected from the group consisting of A587V and A587G (relative to reference sequence SEQ ID NO: 11).

In some embodiments, the capsid protein may comprise two or more amino acid substitutions selected from the group consisting of N452K, N452A, N452V, G453A, G453N, S454T, S454D, G455N, Q456L, Q456K, N457L, N457V, Q458I, and Q458H (relative to reference sequence SEQ ID NO: 11).

In some embodiments, the capsid protein may comprise the amino acid substitution N452K, N452A, or N452V. In some embodiments, the capsid protein comprises N452K substitution relative to reference sequence SEQ ID NO: 211.

In some embodiments, the capsid protein may comprise the amino acid substitution G453A or G453N (relative to reference sequence SEQ ID NO: 11).

In some embodiments, the capsid protein may comprise the amino acid substitution S454T or S454D (relative to reference sequence SEQ ID NO: 11).

In some embodiments, the capsid protein may comprise the amino acid substitution G455N (relative to reference sequence SEQ ID NO: 11).

In some embodiments, the capsid protein may comprise the amino acid substitution Q456L or Q456K (relative to reference sequence SEQ ID NO: 11).

In some embodiments, the capsid protein may comprise the amino acid substitution N457L or N457V (relative to reference sequence SEQ ID NO: 11).

In some embodiments, the capsid protein may comprise the amino acid substitution Q458I or Q458H (relative to reference sequence SEQ ID NO: 11).

In some embodiments, the capsid protein may comprise an amino acid sequence selected from KGSGQNQ (SEQ ID NO: 27), NASGQNQ (SEQ ID NO: 28), NGTGQNQ (SEQ ID NO: 29), NGSGLNQ (SEQ ID NO: 30), ANDNKLI (SEQ ID NO: 31), VNDNKVI (SEQ ID NO: 32), NGSGQNH (SEQ ID NO: 33), and ANDNKVI (SEQ ID NO: 34) at positions 452-458 or at about positions 452-458 relative to reference sequence SEQ ID NO: 11. In some of these embodiments, the capsid protein comprises N452K substitution relative to reference sequence SEQ ID NO: 11.

In some embodiments, the capsid protein at the VR-VIII site comprises comprise an amino acid sequence selected from ENTVSI (SEQ ID NO: 35), QTLFNS (SEQ ID NO: 36), NSTYLG (SEQ ID NO: 37), GSILTH (SEQ ID NO: 38), MMTTAR (SEQ ID NO: 39), and CSTSIR (SEQ ID NO: 40). The capsid protein may comprise an amino acid sequence selected from ENTVSI (SEQ ID NO: 35), QTLFNS (SEQ ID NO: 36), NSTYLG (SEQ ID NO: 37), GSILTH (SEQ ID NO: 38), MMTTAR (SEQ ID NO: 39), and CSTSIR (SEQ ID NO: 40) at positions 585-590 or at about positions 585-590 relative to reference sequence SEQ ID NO: 11. In some of these embodiments, the capsid protein comprises N452K substitution relative to reference sequence SEQ ID NO: 11.

In some embodiments, the capsid protein comprises, at the VR-VIII site, or at amino acid positions 581-594 relative to reference sequence SEQ ID NO: 11, the amino acid sequence ATNHNSTYLGAQTG (SEQ ID NO: 41), and optionally wherein the capsid protein further comprises an amino acid substitution N452K.

In some embodiments, the capsid protein comprises, at the VR-VIII site, or at amino acid positions 581-594 relative to reference sequence SEQ ID NO: 11, the amino acid sequence ATNHMMTTARAQTG (SEQ ID NO: 42), and optionally wherein the capsid protein further comprises an amino acid substitution N452K.

In some embodiments, the capsid protein comprises, at the VR-VIII site, or at amino acid positions 581-594 relative to reference sequence SEQ ID NO: 11, the amino acid sequence ATNHCSTSIRAQTG (SEQ ID NO: 43), and optionally wherein the capsid protein further comprises an amino acid substitution N452K.

In some embodiments, the capsid protein comprises any capsid nucleic acid or amino acid sequence described herein (e.g., see the figures and the sequences presented herein).

In some embodiments, the capsid protein comprises the amino acid sequence of SEQ ID NO: 23, or an amino acid sequence having at least 80%, 85%, 90%, 95% or 98% sequence identity to SEQ ID NO: 23. In some embodiments, the capsid protein is that of ZC375 described herein, or an amino acid sequence having at least 80%, 85%, 90%, 95% or 98% sequence identity to the capsid protein of ZC375 described herein.

In some embodiments, the capsid protein comprises the amino acid sequence of SEQ ID NO: 24, or an amino acid sequence having at least 80%, 85%, 90%, 95% or 98% sequence identity to SEQ ID NO: 24. In some embodiments, the capsid protein is that of ZC401 described herein, or an amino acid sequence having at least 80%, 85%, 90%, 95% or 98% sequence identity to the capsid protein of ZC401 described herein.

In some embodiments, the capsid protein comprises the amino acid sequence of SEQ ID NO: 25, or an amino acid sequence having at least 80%, 85%, 90%, 95% or 98% sequence identity to SEQ ID NO: 25. In some embodiments, the capsid protein is that of ZC428 described herein, or an amino acid sequence having at least 80%, 85%, 90%, 95% or 98% sequence identity to the capsid protein of ZC428 described herein.

In some embodiments, the capsid protein comprises the amino acid sequence of SEQ ID NO: 26, or an amino acid sequence having at least 80%, 85%, 90%, 95% or 98% sequence identity to SEQ ID NO: 26. In some embodiments, the capsid protein is that of ZC478 described herein, or an amino acid sequence having at least 80%, 85%, 90%, 95% or 98% sequence identity to the capsid protein of ZC478 described herein.

In some embodiments, the capsid protein comprises the amino acid sequence of SEQ ID NO: 44, or an amino acid sequence having at least 80%, 85%, 90%, 95% or 98% sequence identity to SEQ ID NO: 44. In some embodiments, the capsid protein is that of ZC96 described herein, or an amino acid sequence having at least 80%, 85%, 90%, 95% or 98% sequence identity to the capsid protein of ZC96 described herein.

In some embodiments, the capsid protein comprises an amino acid sequence of X₁DVQX₂X₃PGFX₄X₅X₆X₇X₈ (SEQ ID NO: 45) at the VR-VIII site (e.g., of an AAV9 capsid protein or a variant thereof), wherein each of X₁, X₂, X₃, X₄, X₅, X₆, X₇, and X₈ is any amino acid.

In some embodiments, X₁ is alanine (A).

In some embodiments, X₂ is glutamine (Q).

In some embodiments, X₇ is threonine (T).

In some embodiments, X₅ is alanine (A) or proline (P).

In some embodiments, X₆ is glutamine (Q) or glutamic acid (E).

In some embodiments, X₄ is glutamine (Q), glycine (G), arginine (R), asparagine (N), histidine (H), methionine (M), proline (P), or serine (S).

In some embodiments, X₈ is glutamic acid (E), methionine (M), glutamine (Q), aspartic acid (D), leucine (L), alanine (A), cysteine (C), histidine (H), phenylalanine (F), tyrosine (Y), threonine (T), valine (V), isoleucine (I), serine (S), or asparagine (N). In some embodiments, X₈ is glutamic acid (E).

In some embodiments, X₃ is leucine (L), histidine (H), valine (V), cysteine (C), glutamine (Q), glycine (G), isoleucine (I), methionine (M), phenylalanine (F), proline (P), threonine (T), or tyrosine (Y).

In some embodiments, X₁ is A, X₂ is Q, X₇ is T, and/or the capsid protein comprises in the VR-VIII site an amino acid sequence of ADVQQX₃PGFX₄X₅X₆TX₈ (SEQ ID NO: 46), wherein each of X₃, X₄, X₅, X₆, and X₈ is any amino acid.

In some embodiments, X₅ is A or P, and X₆ is Q or E, and/or the capsid protein comprises in the VR-VIII site an amino acid sequence of X₁DVQX₂X₃PGFX₄AQX₇X₈ (SEQ ID NO: 47), X₁DVQX₂X₃PGFX₄AEX₇X₈ (SEQ ID NO: 48), X₁DVQX₂X₃PGFX₄PQX₇X₈ (SEQ ID NO: 49), X₁DVQX₂X₃PGFX₄PEX₇X₈ (SEQ ID NO: 50), ADVQQX₃PGFX₄AQTX₈ (SEQ ID NO: 51), ADVQQX₃PGFX₄AETX₈ (SEQ ID NO: 52), ADVQQX₃PGFX₄PQTX₈ (SEQ ID NO: 53), or ADVQQX₃PGFX₄PETX₈ (SEQ ID NO: 54), wherein each of X₁, X₂, X₃, X₄, X₇, and X₈ is any amino acid.

In some embodiments, X₄ is Q, X₅ is A, and X₆ is Q, and/or the capsid protein comprises in the VR-VIII site an amino acid sequence of X₁DVQX₂X₃PGFQAQX₇X₈ (SEQ ID NO: 55) or ADVQQX₃PGFQAQTX₈ (SEQ ID NO: 56), wherein each of X₁, X₂, X₃, X₇, and X₈ is any amino acid.

In some embodiments, X₃ is L, and X₈ is E, and/or the capsid protein comprises in the VR-VIII site an amino acid sequence of X₁DVQX₂LPGFX₄X₅X₆X₇E (SEQ ID NO: 57) or ADVQQLPGFX₄X₅X₆TE (SEQ ID NO: 58), wherein each of X₁, X₂, X₄, X₅, X₆, and X₇ is any amino acid.

In some embodiments, X₄ is Q, and/or the capsid protein comprises in the VR-VIII site an amino acid sequence of X₁DVQX₂X₃PGFQX₅X₆X₇X₈ (SEQ ID NO: 59) or ADVQQX₃PGFQX₅X₆TX₈ (SEQ ID NO: 60), wherein each of X₁, X₂, X₃, X₅, X₆, X₇, and X₈ is any amino acid.

In some embodiments, the capsid protein comprises, consists essentially of, or consists of a sequence having at least about 80% (e.g., at least 80%, 85%, 90%, 95%, 96%, 97%, 98%, 99%, 99.5%, or 100%) identity to any one of the following sequences at the VR-VIII site (positions 581-594 relative to reference sequence SEQ ID NO: 11), with up to 1, 2, or 3 substitutions: ADVQQLPGFQAQTE (SEQ ID NO: 61), ADVQQHPGFQAQTE (SEQ ID NO: 62), ADVQQVPGFQAQTM (SEQ ID NO: 63), ADVQQVPGFQAQTQ (SEQ ID NO: 64), ADVQQLPGFGAQTE (SEQ ID NO: 65), ADVQQLPGFRPETE (SEQ ID NO: 66), and ADVQQLPGFNAQTE (SEQ ID NO: 67).

In some embodiments, the capsid protein comprises any substitution and/or insertion motif described herein. In some embodiments, the capsid protein comprises a substitution motif having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 95%, 96%, 97%, 98%, 99%, or 100% sequence identity to any substitution motif described herein. In some embodiments, the capsid protein comprises an insertion motif having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 95%, 96%, 97%, 98%, 99%, or 100% sequence identity to any insertion motif described herein.

It should be noted that the above modified VR-VIII motifs are described in the context of AAV9 capsid proteins for illustrative purposes only and are not meant to be limited to AAV9 capsid proteins. Instead, any modified VR-VIII motif described herein can be applied to other AAV capsid proteins of a different serotype (e.g., AAV5, AAVrh.10, or AAVrh.74), for example, by replacing the wild-type sequence at the VR-VIII site of the corresponding capsid protein (e.g., amino acid positions 570 to 583 of wild-type AAV5 VP1 capsid protein sequence according to SEQ ID NO: 19, amino acid positions 583 to 596 of wild-type AAVrh.10 VP1 capsid protein sequence, or amino acid positions 583 to 596 of wild-type AAVrh.74 VP1 capsid protein sequence) with any of the modified VR-VIII motifs described herein to generate a variant of the capsid protein of a particular serotype. In some embodiments, the capsid protein is a variant of an AAV5, AAV9, AAVth.10, or AAVrh.74 capsid protein.

In some embodiments, the capsid protein comprises an insertion polypeptide or insertion motif compared to the wild-type or parental capsid protein. In some embodiments, the capsid protein additionally comprises one or more amino acid substitutions in the amino acid sequence of the wild-type or parental capsid protein sequence from which it is derived. In some embodiments, the insertion motif is inserted at a surface loop region of the capsid protein, for example, at a VR-IV, VR-V, VR-VII and/or VR-VIII site, as described.

In some embodiments, the insertion motif comprises or consists of an amino acid sequence of “RGDAARL” (SEQ ID NO: 68); and/or the capsid protein comprises an amino acid sequence of “RGDAARL” (SEQ ID NO: 68).

In some embodiments, the insertion motif comprises or consists of an amino acid sequence of “SHVRGDL” (SEQ ID NO: 69); and/or the capsid protein comprises an amino acid sequence of “SHVRGDL” (SEQ ID NO: 69).

In some embodiments, the insertion motif comprises or consists of an amino acid sequence of “VVSSGAR” (SEQ ID NO: 70); and/or the capsid protein comprises an amino acid sequence of “VVSSGAR” (SEQ ID NO: 70).

In some embodiments, the insertion motif comprises or consists of an amino acid sequence of “VRGD” (SEQ ID NO: 71); and/or the capsid protein comprises an amino acid sequence of “VRGD” (SEQ ID NO: 71).

The insertion motif can occur (e.g., be inserted) at any position of the capsid protein, for example, at a surface or an exposed region of the capsid protein. In some embodiments, the engineered AAV capsid protein comprises an insertion motif as described herein inserted at a surface loop region of the capsid protein, e.g., the VR-I, VR-II, VR-IV, VR-V, VR-VII, and/or VR-VIII site of the capsid protein. In some embodiments, the engineered AAV capsid protein comprises an insertion motif as described inserted at the VR-IV and/or the VR-VIII site of the capsid protein. In certain of these embodiments, the engineered capsid protein additionally comprises one or more amino acid substitutions in the same VR site as the insertion or at a different location from the insertion.

In some embodiments, the capsid protein is a variant AAV9 capsid protein that comprises an insertion polypeptide or insertion motif at the VR-VIII site, e.g., between amino acids 588 (glutamine (Q)) and 589 (alanine (A)) within the VR-VIII site in reference to the wild-type full-length AAV9 capsid protein of SEQ ID NO: 11. In some embodiments, the variant AAV9 capsid protein further comprises one or more amino acid substitutions within the VR-VIII site, including, for example, at one or more of amino acid positions 587-590 in reference to the wild-type full-length AAV9 capsid protein of SEQ ID NO: 11. In certain of these embodiments, the insertion motif comprises an amino acid sequence of RGDAARL (SEQ ID NO: 68), RTDLKGL (SEQ ID NO: 72), YPSTGSG (SEQ ID NO: 73), FAGSLTRA (SEQ ID NO: 74), DRTLTTR (SEQ ID NO: 75), RIAGRDV (SEQ ID NO: 76), or SLGSGVR (SEQ ID NO: 77).

In some embodiments, the capsid protein is an engineered AAV9 capsid protein that comprises an insertion polypeptide or insertion motif at the VR-IV site, e.g., between amino acids 453 (glycine (G)) and 454 (serine (S)), and/or between amino acids 456 (glutamine (Q)) and 457 (asparagine (N)), within the VR-IV site in reference to the wild-type full-length AAV9 capsid protein of SEQ ID NO: 11. In certain of these embodiments, the insertion motif comprises an amino acid sequence of SHVRGDL (SEQ ID NO: 69), VVSSGAR (SEQ ID NO: 70), PQYGRGG (SEQ ID NO: 78), LQVSRVS (SEQ ID NO: 79), VRSYSSN (SEQ ID NO: 80), “TMRVGSL” (SEQ ID NO: 81), GAYSRGV (SEQ ID NO: 82), LRGGSLG (SEQ ID NO: 83), or “VYGTGVR” (SEQ ID NO: 84).

In some embodiments, the capsid protein is an engineered AAV5 capsid protein that comprises an insertion polypeptide or insertion motif at the VR-VIII site, e.g., between amino acids 574 (glutamine (Q)) and 575 (serine (S)) within the VR-VIII site in reference to the wild-type full-length AAV5 capsid protein of SEQ ID NO: 19. In certain of these embodiments, the insertion motif comprises an amino acid sequence of DKLIIVS (SEQ ID NO: 85), AEDRTKL (SEQ ID NO: 86), LSASASL (SEQ ID NO: 87), LADQTKL (SEQ ID NO: 88), LLLKLQE (SEQ ID NO: 89), ELPVKTG (SEQ ID NO: 90), LDLKVVG (SEQ ID NO: 91), or RDAVL (SEQ ID NO: 92).

In some embodiments, the capsid protein comprises the amino acid sequence of SEQ ID NO: 93, or an amino acid sequence having at least 80%, 85%, 90%, 95% or 98% sequence identity to SEQ ID NO: 93. In some embodiments, the capsid protein is that of ZC621 described herein, or an amino acid sequence having at least 80%, 85%, 90%, 95% or 98% sequence identity to the capsid protein of ZC621 described herein.

In some embodiments, the capsid protein comprises the amino acid sequence of SEQ ID NO: 94, or an amino acid sequence having at least 80%, 85%, 90%, 95% or 98% sequence identity to SEQ ID NO: 94. In some embodiments, the capsid protein is that of ZC623 described herein, or an amino acid sequence having at least 80%, 85%, 90%, 95% or 98% sequence identity to the capsid protein of ZC623 described herein.

In some embodiments, the capsid protein comprises the amino acid sequence of SEQ ID NO: 95, or an amino acid sequence having at least 80%, 85%, 90%, 95% or 98% sequence identity to SEQ ID NO: 95. In some embodiments, the capsid protein is that of ZC625 described herein, or an amino acid sequence having at least 80%, 85%, 90%, 95% or 98% sequence identity to the capsid protein of ZC625 described herein.

In some embodiments, the capsid protein comprises the amino acid sequence of SEQ ID NO: 96, or an amino acid sequence having at least 80%, 85%, 90%, 95% or 98% sequence identity to SEQ ID NO: 96. In some embodiments, the capsid protein is that of ZC626 described herein, or an amino acid sequence having at least 80%, 85%, 90%, 95% or 98% sequence identity to the capsid protein of ZC626 described herein.

In some embodiments, the capsid protein comprises the amino acid sequence of SEQ ID NO: 97, or an amino acid sequence having at least 80%, 85%, 90%, 95% or 98% sequence identity to SEQ ID NO: 97. In some embodiments, the capsid protein is that of ZC630 described herein, or an amino acid sequence having at least 80%, 85%, 90%, 95% or 98% sequence identity to the capsid protein of ZC630 described herein.

In some embodiments, the capsid protein comprises the amino acid sequence of SEQ ID NO: 98, or an amino acid sequence having at least 80%, 85%, 90%, 95% or 98% sequence identity to SEQ ID NO: 98. In some embodiments, the capsid protein is that of ZC631 described herein, or an amino acid sequence having at least 80%, 85%, 90%, 95% or 98% sequence identity to the capsid protein of ZC631 described herein.

In some embodiments, the capsid protein comprises the amino acid sequence of SEQ ID NO: 99, or an amino acid sequence having at least 80%, 85%, 90%, 95% or 98% sequence identity to SEQ ID NO: 99. In some embodiments, the capsid protein is that of ZC632 described herein, or an amino acid sequence having at least 80%, 85%, 90%, 95% or 98% sequence identity to the capsid protein of ZC632 described herein.

In some embodiments, the capsid protein comprises the amino acid sequence of SEQ ID NO: 100, or an amino acid sequence having at least 80%, 85%, 90%, 95% or 98% sequence identity to SEQ ID NO: 100. In some embodiments, the capsid protein is that of ZC633 described herein, or an amino acid sequence having at least 80%, 85%, 90%, 95% or 98% sequence identity to the capsid protein of ZC633 described herein.

In some embodiments, the capsid protein comprises the amino acid sequence of SEQ ID NO: 101, or an amino acid sequence having at least 80%, 85%, 90%, 95% or 98% sequence identity to SEQ ID NO: 101. In some embodiments, the capsid protein is that of ZC634 described herein, or an amino acid sequence having at least 80%, 85%, 90%, 95% or 98% sequence identity to the capsid protein of ZC634 described herein.

In some embodiments, the capsid protein comprises the amino acid sequence of SEQ ID NO: 102, or an amino acid sequence having at least 80%, 85%, 90%, 95% or 98% sequence identity to SEQ ID NO: 102. In some embodiments, the capsid protein is that of ZC635 described herein, or an amino acid sequence having at least 80%, 85%, 90%, 95% or 98% sequence identity to the capsid protein of ZC635 described herein.

In another aspect, the disclosure provides recombinant adeno-associated virus (rAAV) virions comprising any of the capsid proteins described herein and a vector genome. The vector genome may comprise an expression cassette flanked by inverted terminal repeats (ITRs). The expression cassette may comprise a nucleic acid sequence encoding a therapeutic gene product.

### Compositions and Kits

In some embodiments, the present disclosure provides compositions comprising isolated viral particles (e.g., rAAV) produced in accordance with the methods described herein (e.g., purified viral particles). In some embodiments, the present disclosure provides a pharmaceutically acceptable composition comprising isolated and purified viral particles (e.g., rAAV) produced in accordance with the methods described herein, and a pharmaceutically acceptable carrier.

In some embodiments, the present disclosure provides a kit comprising (i) one or more HDAC6 inhibitors described herein, (ii) one or more virus, viral vector or plasmid described herein (e.g., a vector carrying a transgene, a plasmid carrying helper genes, and/or plasmid(s) carrying genes necessary for viral replication and/or encapsidation); (iii) one or more cells described herein, and/or (iv) cell culture media; or any combination of (i)-(iv).

### HDAC6 Inhibitors for Use in the Methods Described Herein

Histone deacetylases (“HDAC”) are a class of enzymes with deacetylase activity with a broad range of genomic and non-genomic substrates. There are eleven Zinc-dependent HDAC enzymes classified based on sequence identity and catalytic activity (Haberland et al., 2009).

Histone deacetylase inhibitors have been described as a therapeutic agents in oncology (Yoon and Eom, 2016), neurodegeneration (Butler et al., 2010) autoimmune disease (Choi et al., 2018), chemotherapy-induced peripheral neuropathy (Krukowski et al., 2017) and cardiac indications (Zhang et al., 2002). Given the role of nuclear HDACs on regulating gene transcription, inhibition of these class of targets is known to have pleiotropic effects in various cell types; most notably resulting in cell toxicities. Therefore, limiting the toxicity of pan-HDAC inhibitors has been a major obstacle in wide-spread utilization for this class of compounds. In addition, significant adverse effects of pan-HDAC inhibitors (e.g. SAHA and Panabinostat) has been observed in the clinic including fatigue, nausea, diarrhea and thrombocytopenia (Subramanian et al., 2010).

HDAC6 belongs to the class IIb enzyme and contains two catalytic domains, a ubiquitin binding domain and a cytoplasmic retention domain (Haberland et al., 2009). HDAC6 is predominately a cytoplasmic enzyme and its best-characterized substrates include tubulin, HSP90 and cortactin (Brindisi et al., 2020).

Pharmacological inhibition of HDAC6 blocks its deacetylase activity, thus resulting in hyperacetylation of its substrates, most notably tubulin (Hubbert et al., 2002).

HDAC6-selective inhibitors are known to have reduced cytotoxicity due to the cytoplasmic nature of HDAC6 substrates and reduced effects on nuclear targets (including H3K9 and c-MYC) and on global transcription (Nebbioso et al., 2017).

Hydroxamic acids are zinc chelators and have been used extensively in the development of pan- and HDAC-selective inhibitors. However, most hydroxamic-acid based HDAC inhibitors either lack the desired selectivity or show poor bioavailability with a poor pharmacokinetic profile (Butler et al., 2010; Santo et al., 2012).

In some embodiments, the HDAC6 inhibitor used in the methods described herein is any HDAC6 inhibitor described herein.

In some embodiments, the HDAC6 inhibitor used in the methods described herein is a selective HDAC6 inhibitor.

Various selective HDAC6 are known in the art. In addition, using known methods it is routine to screen compounds to identify further selective HDAC6 inhibitors. In particular, given a known HDAC6 inhibitor, a person of skill in the art can identify which analogs of the compound have selective HDAC6 activity.

In some embodiments, the HDAC6 inhibitor used in the methods described herein is any selective HDAC6 inhibitor known in the art.

In some embodiments, the HDAC6 inhibitor used in the methods described herein is not a non-selective HDAC6 inhibitor. In some embodiments, the HDAC6 inhibitor used in the methods described herein is not a pan-HDAC inhibitor.

In some embodiments, two or more of the selective HDAC6 inhibitors described herein are used in combination.

### Known HDAC6 Inhibitors

In some embodiments, the TDAC6 inhibitor is CAY10603, tubacin, ricolinostat (ACY-1215), citarinostat (ACY-241), ACY-738, ACY-775, ACY-1083, QTX-153, QTX-125, KA2507, A452, WT161, BRD73954, JBI-802, nexturastat A, tubastatin A, HPOB, SKLB-23bb, CKD-504, CKD-506, CKD-509, CKD-510, CS3003, ITF-3753, AJ302, or an analog thereof. In some embodiments, the DAC6 inhibitor is rocilinostat (ACY-1215), citarinostat (ACY-241), KA2507, CKD-504, CKD-506, CKD-509, CKD-510, CS3003, or an analog thereof.

In some embodiments, the HDAC6 inhibitor is a compound having the structure:

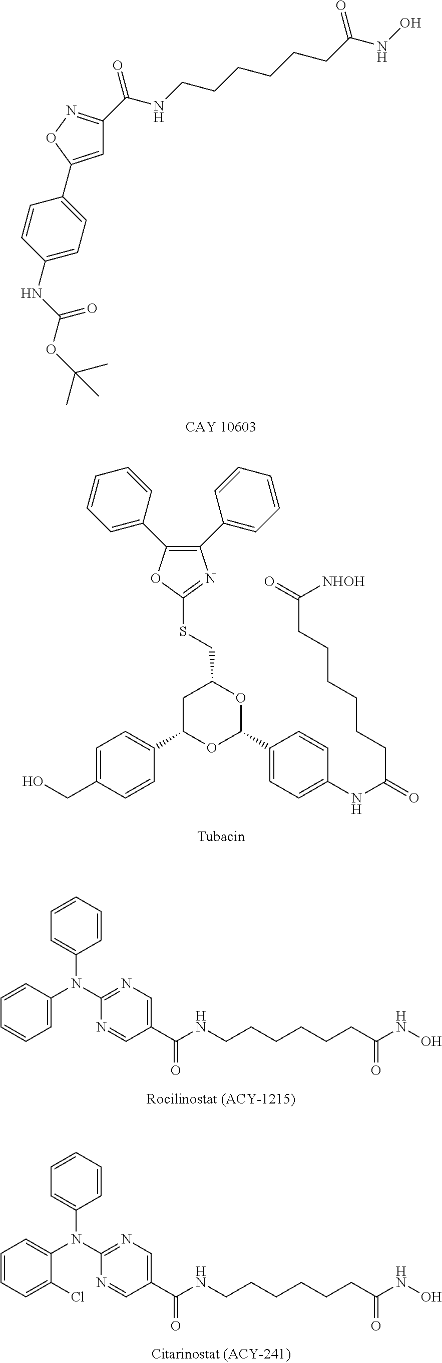

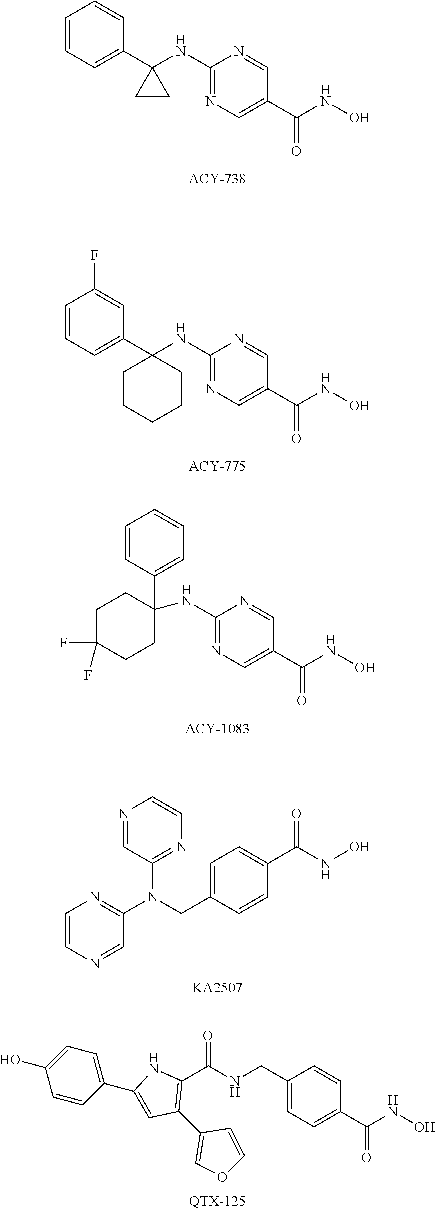

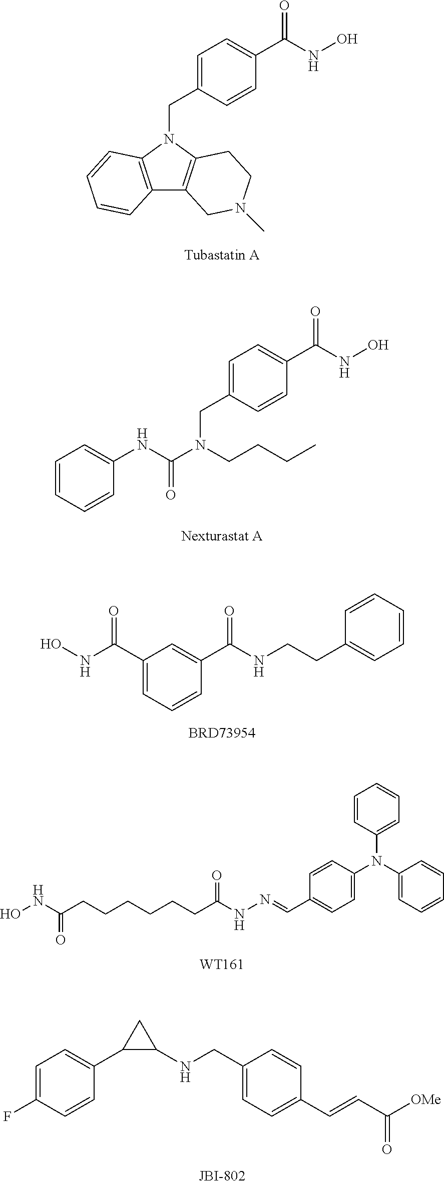

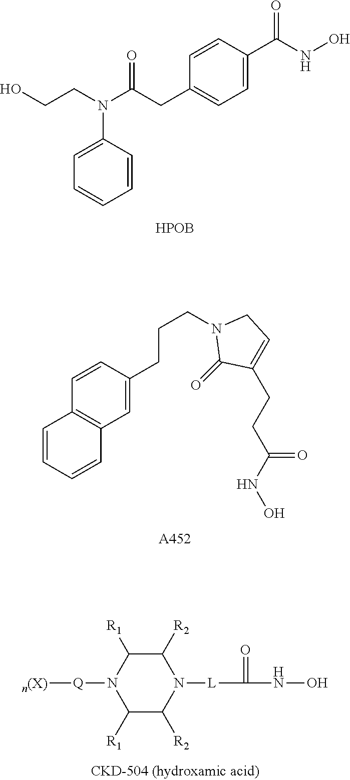

or a salt thereof.

TABLE 1
Non-limiting List of Known HDAC6 Inhibitors and Properties Thereof

| Compound | Description | IC₅₀ | |
|---|---|---|---|
| CAY10603 | CAY10603 is a potent and selective HDAC6 inhibitor | 2 | pM |
| | with IC50 of 2 pM, >200-fold selectivity over other HDACs. | | |
| Tubacin | Tubacin is a highly potent and selective, reversible, cell- | 4 | nM |
| | permeable HDAC6 inhibitor with an IC50 of 4 nM, approximately | | |
| | 350-fold selectivity over HDAC1. | | |
| Ricolinostat | Rocilinostat (ACY-1215) is a selective HDAC6 inhibitor | 4.7 | nM |
| (ACY-1215) | with IC50 of 5 nM. It is >10-fold more selective for HDAC6 than | | |
| | HDAC1/2/3 (class I HDACs) with slight activity against HDAC8, | | |
| | minimal activity against HDAC4/5/7/9/11, Sirtuin1, and Sirtuin2. | | |
| | Phase 2. | | |
| Citarinostat | Citarinostat (ACY-241, HDAC-IN-2) is an orally available | 2.6 | nM |
| (ACY-241) | selective HDAC6 inhibitor with IC50 of 2.6 nM and 46 nM for | | |
| | HDAC6 and HDAC3, respectively. It has 13 to 18-fold selectivity | | |
| | towards HDAC6 in comparison to HDAC1-3. | | |
| KA2507 | KA2507 is a highly selective and potent HDAC6 inhibitor. | 2.5 | nM |
| ACY-738 | ACY-738 inhibits HDAC6 with low nanomolar potency (IC50 = 1.7 | 1.7 | nM |
| | nM) and a selectivity of 60- to 1500-fold over class I HDACs. | | |
| QTX-125 | HDAC6 inhibitor with 11-fold selectivity over HDAC1 | 0.6 | nM |
| CKD-506 | HDAC6 inhibitor with an 80-fold selectivity over HDAC1 | 2.9 | nM |
| Nexturastat A | Nexturastat A is a potent and selective HDAC6 inhibitor | 5 | nM |
| | with IC50 of 5 nM, >190-fold selectivity over other HDACs. | | |
| Tubastatin A | Tubastatin A is a potent and selective HDAC6 inhibitor | 15 | nM |
| | with IC50 of 15 nM. It is selective against all the other isozymes | | |
| | (1000-fold) except HDAC8 (57-fold). | | |
| HPOB | HPOB is a potent, selective HDAC6 inhibitor with IC50 of | 56 | nM |

56 nM, >30-fold selectivity over other HDACs.

Further illustrative HDAC6 inhibitors are provided in U.S. Patent Publications Nos. U.S. Pat. No. 8,227,516B2, US20100292169A1, US20070207950A1, U.S. Pat. No. 8,222,423B2, US20100093824A1, US20100216796A1, U.S. Pat. No. 8,673,911B2, U.S. Pat. No. 8,217,076B2, U.S. Pat. No. 8,440,716B2, US20110195432A1, U.S. Pat. No. 8,624,040B2, U.S. Pat. No. 9,096,518B2, U.S. Pat. No. 8,431,538B2, US20120258993A1, U.S. Pat. No. 8,546,588B2, U.S. Pat. No. 8,513,421B2, US20140031368A1, US20120015943A1, US20120015942A1, US20140243335A1, US20130225543A1, U.S. Pat. No. 8,471,026B2, U.S. Pat. No. 9,238,028B2, U.S. Pat. No. 8,765,773B2, USRE47009E1, US20140294856A1, U.S. Pat. No. 9,512,083B2, U.S. Pat. No. 9,670,193B2, U.S. Pat. No. 9,345,905B2, U.S. Pat. No. 9,409,858B2, U.S. Pat. No. 9,663,825B2, US20150119327A1, US20150250786A1, U.S. Ser. No. 10/041,046B2, U.S. Pat. No. 9,586,973B2, US20160069887A1, US20140357512A1, U.S. Pat. No. 9,751,832B2, US20160228434A1, US20150105358A1, U.S. Ser. No. 10/660,890B2, US20160271083A1, US20150176076A1, US20200405716A1, U.S. Pat. No. 9,890,136B2, U.S. Ser. No. 10/287,255B2, US20170173083A1, U.S. Ser. No. 10/016,421B2, U.S. Pat. No. 9,987,258B2, U.S. Ser. No. 10/568,854B2, U.S. Ser. No. 10/106,540B2, U.S. Ser. No. 10/266,489B2, U.S. Pat. No. 9,993,459B2, U.S. Ser. No. 10/183,934B2, U.S. Ser. No. 10/494,354B2, U.S. Ser. No. 10/494,353B2, U.S. Ser. No. 10/112,915B2, U.S. Ser. No. 10/377,726B2, U.S. Ser. No. 10/829,462B2, U.S. Ser. No. 10/829,461B2, US20210009539A1, US20210009538A1, U.S. Ser. No. 10/239,845B2, U.S. Ser. No. 10/472,337B2, U.S. Ser. No. 10/479,772B2, U.S. Ser. No. 10/464,911B2, U.S. Ser. No. 10/584,117B2, U.S. Ser. No. 10/538,498B2, U.S. Ser. No. 10/011,611B2, U.S. Ser. No. 10/494,355B2, U.S. Ser. No. 10/040,769B2, U.S. Ser. No. 10/858,323B2, U.S. Ser. No. 10/654,814B2, US20190209559A1, US20190185462A1, US20190192521A1, US20190321361A1, US20200046698A1, US20190262337A1, US20190282573A1, US20190282574A1, US20200071288A1, U.S. Ser. No. 10/745,389B2, U.S. Ser. No. 10/357,493B2, US20200171028A1, US20200054773A1, US20200308174A1, US20200155549A1, U.S. Ser. No. 10/435,399B2, US20200216563A1, US20190216751A1, US20200339569A1, US20210078963A1, US20210077487A1, US20190270733A1, US20190270744A1, US20200022966A1, and US20210094944A1, which are incorporated herein for purposes of identifying HDAC6 inhibitors that may be used in the methods disclosed herein.

In some embodiments, the selective HDAC6 inhibitor used in the methods described herein is more selective against HDAC6 compared to any other (e.g., HDAC1) or all other isozymes of HDAC (i.e., inhibits HDAC6 more than other HDACs) by at least 15 fold, 20 fold, 30 fold, 40 fold, 50 fold, 60 fold, 70 fold, 80 fold, 90 fold, 100 fold, 110 fold, 120 fold, 130 fold, 140 fold, 150 fold, 160 fold, 170 fold, 180 fold, 190 fold, 200 fold, 210 fold, 220 fold, 230 fold, 240 fold, 250 fold, 300 fold, 350 fold, 400 fold, 450 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10000 fold, 15000 fold, 20000 fold, 25000 fold, 30000 fold, or more. In some embodiments, the HDAC6 inhibitor used in the methods described herein selectively inhibits HDAC6 over any one or all other HDACs (e.g., HDAC1) by at least 20 fold. In some embodiments, the HDAC6 inhibitor used in the methods described herein selectively inhibits HDAC6 over any one or all other HDACs (e.g., HDAC1) by at least 30 fold. In some embodiments, the HDAC6 inhibitor used in the methods described herein selectively inhibits HDAC6 over any one or all other HDACs (e.g., HDAC1) by at least 40 fold. In some embodiments, the HDAC6 inhibitor used in the methods described herein selectively inhibits HDAC6 over any one or all other HDACs (e.g., HDAC1) by at least 50 fold. In some embodiments, the HDAC6 inhibitor used in the methods described herein selectively inhibits HDAC6 over any one or all other HDACs (e.g., HDAC1) by at least 60 fold. In some embodiments, the HDAC6 inhibitor used in the methods described herein selectively inhibits HDAC6 over any one or all other HDACs (e.g., HDAC1) by at least 100 fold. In some embodiments, the HDAC6 inhibitor used in the methods described herein selectively inhibits HDAC6 over any one or all other HDACs (e.g., HDAC1) by at least 200 fold. In some embodiments, the HDAC6 inhibitor used in the methods described herein selectively inhibits HDAC6 over any one or all other HDACs (e.g., HDAC1) by at least 500 fold. In some embodiments, the HDAC6 inhibitor used in the methods described herein selectively inhibits HDAC6 over any one or all other HDACs (e.g., HDAC1) by at least 1000 fold. In some embodiments, the HDAC6 inhibitor used in the methods described herein selectively inhibits HDAC6 over any one or all other HDACs (e.g., HDAC1) by at least 2000 fold. In some embodiments, the HDAC6 inhibitor used in the methods described herein selectively inhibits HDAC6 over any one or all other HDACs (e.g., HDAC1) by at least 5000 fold.

In some embodiments, the selective HDAC6 inhibitor used in the methods described herein inhibits HDACs other than HDAC6 at least 10-fold less effectively, at least 20-fold less effectively, at least 30-fold less effectively, at least 40-fold less effectively, at least 50-fold less effectively, at least 60-fold less effectively, at least 70-fold less effectively, at least 80-fold less effectively, at least 90-fold less effectively, at least 100-fold less effectively, at least 200-fold less effectively, at least 500-fold less effectively, at least 1000-fold less effectively, at least 5000-fold less effectively, or at least 10000-fold less effectively, than a pan-HDAC inhibitor, e.g., givinostat. In some embodiments, the selective HDAC6 inhibitor used in the methods described herein inhibits HDACs other than HDAC6 at least 100-fold less effectively than givinostat. In some embodiments, the selective HDAC6 inhibitor used in the methods described herein inhibits HDACs other than HDAC6 at least 500-fold less effectively than givinostat. In some embodiments, the selective HDAC6 inhibitor used in the methods described herein inhibits HDACs other than HDAC6 at least 1000-fold less effectively than givinostat. In some embodiments, the selective HDAC6 inhibitor used in the methods described herein inhibits HDACs other than HDAC6 at least 5000-fold less effectively than givinostat.

In some embodiments, the HDAC6 inhibitor is not a non-selective inhibitor of HDAC6. In some embodiments, the HDAC6 inhibitor is not a pan-HDAC inhibitor. In some embodiments, the HDAC6 inhibitor is not a hydroxamic acid. In some embodiments, the HDAC6 inhibitor is not rocilinostat.

In some embodiments, the methods described herein do not include using a non-selective inhibitor of HDAC6. In some embodiments, the methods described herein do not include using a pan-HDAC inhibitor. In In some embodiments, the methods described herein do not include using hydroxamic acid. In some embodiments, the methods described herein do not include using rocilinostat.

### Fluoroalkyl-Oxadiazole Derivatives

In some embodiments, the HDAC6 inhibitor used in the methods described herein is a fluoroalkyl-oxadiazole derivative. Illustrative fluoroalkyl-oxadiazole derivatives that may be used as HDAC6 inhibitors include those described herein and those described in Int′l Pat. Appl. No. PCT/US2020/066439, published as WO2021127643A1 the content of which is incorporated by reference herein in its entirety. PCT/US2020/066439, published as WO2021127643A1, also describes methods of synthesis of such compounds, which are specifically incorporated by reference herein.

In some embodiments, the HDAC6 inhibitor is a compound of Formula (I):

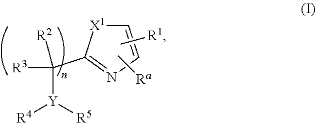

wherein
R¹ is selected from the group consisting of:

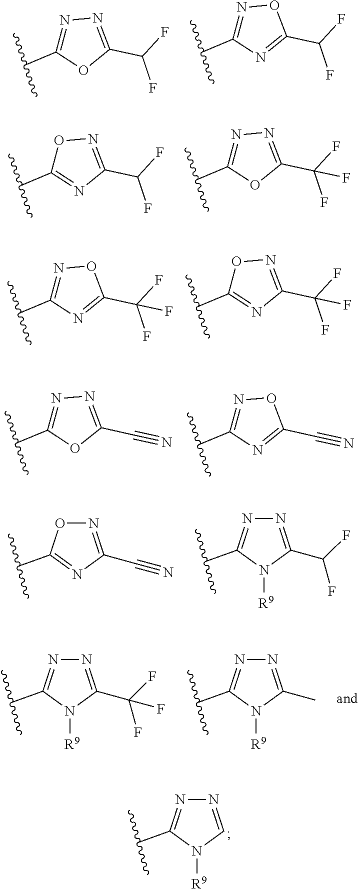

R^{a} is selected from the group consisting of H, halo, C₁₋₃ alkyl, cycloalkyl, haloalkyl, and alkoxy;
R² and R³ are independently selected from the group consisting of H, halogen, alkoxy, haloalkyl, aryl, heteroaryl, alkyl, and cycloalkyl each of which is optionally substituted, or R² and R³ together with the atom to which they are attached form a cycloalkyl or heterocyclyl;
R⁴ and R⁵ are independently selected from the group consisting of H, —(SO₂)R², —(SO₂)NR²R³, —(CO)R², —(CONR²R³), aryl, arylheteroaryl, alkylenearyl, heteroaryl, cycloalkyl, heterocyclyl, alkyl, haloalkyl, and alkoxy, each of which is optionally substituted, or R⁴ and R⁵ together with the atom to which they are attached form a cycloalkyl or heterocyclyl, each of which is optionally substituted;
R⁹ is selected from the group consisting of H, C₁-C₆ alkyl, haloalkyl, cycloalkyl and heterocyclyl;
X¹ is selected from the group consisting of S, O, NH and NR⁶, wherein R⁶ is selected from the group consisting of C₁-C₆ alkyl, alkoxy, haloalkyl, cycloalkyl and heterocyclyl;
Y is selected from the group consisting of CR², O, N, S, SO, and SO₂, wherein when Y is 0, S, SO, or SO₂, R⁵ is not present and when R⁴ and R⁵ together with the atom to which they are attached form a cycloalkyl or heterocyclyl, Y is CR² or N; and n is selected from 0, 1, and 2.

In some embodiments of Formula (I), n is 0. In some embodiments, n is 1. In some embodiments n is 2. In some embodiments, n is 0 or 1. In some embodiments n is 1 or 2. In some embodiments n is 0 or 2.

In some embodiments of Formula (I), X¹ is O. In some embodiments, X¹ is S. In some embodiments, X¹ is NH. In some embodiments, X¹ is NR⁶. In some embodiments, X¹ is selected from the group consisting of S, O, and NR⁶. In some embodiments, X¹ is selected from the group consisting of S, O, and NCH₃. In some embodiments, X¹ is S or O. In some embodiments, X¹ is S or NR⁶. In some embodiments, R⁶ is C₁-C₆ alkyl.

In some embodiments of Formula (I), R² and R³ are H.

In some embodiments of Formula (I), Y is N, CR², or O. In some embodiments, Y is N or O. In some embodiments, Y is N. In some embodiments, Y is CR². In some embodiments, Y is O.

In some embodiments, R⁴ and R⁵ are independently selected from the group consisting of H, —(SO₂)R², —(SO₂)NR²R³, —(CO)R², —(CONR²R³), aryl, arylheteroaryl, heteroaryl, alkylenearyl, cycloalkyl, alkylenecycloalkyl, heterocyclyl, alkyleneheterocyclyl, alkyl, haloalkyl, and alkoxy, each of which is optionally substituted, or R⁴ and R⁵ together with the atom to which they are attached form a cycloalkyl or heterocyclyl, each of which is optionally substituted

In some embodiments of Formula (I), R⁴ is selected from the group consisting of —C(O)— alkyl, —C(O)-cycloalkyl, —C(O)-aryl, —C(O)-heteroaryl, —(SO₂)NR²R³, —SO₂-alkyl, and —SO₂-cycloalkyl, each of which is optionally substituted. In some embodiments, R⁴ is selected from the group consisting of —C(O)-alkyl, —C(O)-cycloalkyl, —SO₂-alkyl, —SO₂-haloalkyl, —SO₂-cycloalkyl, and —(SO₂)NR²R³, each of which is optionally substituted. In some embodiments, aryl is optionally substituted with one or more halogens. In some embodiments of Formula (I), R⁴ is selected from the group consisting of —SO₂alkyl, —SO₂haloalkyl, or —SO₂cycloalkyl. In some embodiments of Formula (I), R⁴ is selected from the group consisting of —SO₂Me, —SO₂Et, and —SO₂-cPr. In some embodiments, R² and R³ are each independently —C₁₋₅alkyl. In some embodiments, R² and R³ taken together with the nitrogen atom to which they are attached form an optionally substituted heterocyclyl. In some embodiments, the optionally substituted heterocyclyl is morpholine, thiomorpholine, or thiomorpholine 1,1-dioxide.

In some embodiments of Formula (I), R⁵ is selected from the group consisting of H, —(SO₂)R², —(SO₂)NR²R³, —(CO)R², —(CONR²R³), aryl, arylheteroaryl, alkylenearyl, heteroaryl, cycloalkyl, heterocyclyl, alkyl, haloalkyl, and alkoxy, each of which is optionally substituted.

In some embodiments, R⁵ is aryl, heteroaryl, or cycloalkyl, each of which is optionally substituted.

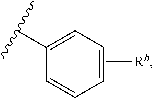

In some embodiments, R⁵ is aryl. In some embodiments, aryl is, wherein R^{b} is one or more selected from the group consisting of hydrogen, halogen, haloalkyl, alkyl, Oalkyl, Ohaloalkyl, alkylene-Ohaloalkyl, cycloalkyl, heterocyclyl aryl, heteroaryl, alkylnitrile, or CN. In some embodiments, the haloalkyl is selected from CF₃, CF₂CH₃, CHF₂, or CH₂F. In some embodiments, the alkyl is a —C₁₋₅alkyl. In some embodiments, —C₁₋₅alkyl is methyl, ethyl, propyl, i-propyl, butyl, or t-butyl. In some embodiments, methyl, ethyl, propyl, i-propyl, butyl, or t-butyl is optionally substituted with OH. In some embodiments, the cycloalkyl is a C₃₋₆cycloalkyl. In some embodiments, the aryl is a phenyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the Ohaloalkyl is selected from OCF₃, OCHF₂, or OCH₂F. In some embodiments, the Oalkyl is O-methyl, O-ethyl, O-propyl, O-i-propyl, O-butyl, or O-t-butyl.

In some embodiments, R⁵ is heteroaryl. In some embodiments, heteroaryl is an optionally substituted 5- to 14-membered heteroaryl. In some embodiments, heteroaryl is an optionally substituted 5- to 14-membered heteroaryl having 1, 2, or 3 heteroatoms selected from the group consisting of N, O, and S. In some embodiments, the optionally substituted 5 to 14-membered heteroaryl is selected from the group consisting of pyrazolyl, imidazolyl, oxazolyl, thiazolyl, pyridinyl, pyrimidinyl, pyrazinyl, pyridazinyl, quinolinyl, isoquinolinyl, quinoxalinyl, cinnolinyl, indolizinyl, azaindolizinyl, indolyl, azaindolyl, benzoxazolyl, benzthiazolyl, benzfuranyl, benzthiophenyl, imidazopyridinyl, imidazopyrazinyl, and benzimidazolyl. In some embodiments, the optionally substituted 5- to 14-membered heteroaryl is selected from the group consisting of pyrazolyl, pyridinyl, pyrimidinyl, pyrazinyl, pyridazinyl, benzoxazolyl, imidazopyridinyl, and imidazopyrazinyl. In some embodiments, R⁵

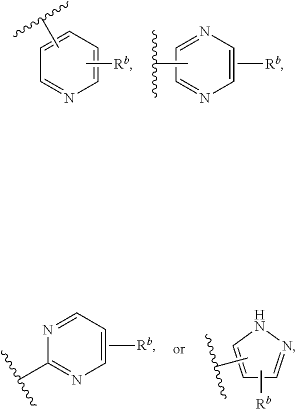

wherein R^{b} is one or more selected from the group consisting of halogen, haloalkyl, alkyl, Oalkyl, Ohaloalkyl, alkylene-Ohaloalkyl, cycloalkyl, heterocyclyl aryl, heteroaryl, alkylnitrile, or CN. In some embodiments, the haloalkyl is selected from CF₃, CF₂CH₃, CHF₂, or CH₂F. In some embodiments, the alkyl is a —C₁₋₅alkyl. In some embodiments, —C₁₋₅alkyl is methyl, ethyl, propyl, i-propyl, butyl, or t-butyl. In some embodiments, methyl, ethyl, propyl, i-propyl, butyl, or t-butyl is optionally substituted with OH. In some embodiments, the cycloalkyl is a C₃₋₆cycloalkyl. In some embodiments, the aryl is a phenyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the Ohaloalkyl is selected from OCF₃, OCHF₂, or OCH₂F. In some embodiments, the Oalkyl is O-methyl, O-ethyl, O-propyl, O-i-propyl, O-butyl, or O-t-butyl.

In some embodiments, R⁵ is cycloalkyl. In some embodiments, cycloalkyl is a cyclopropyl, cyclobutyl, cyclopentyl, or cyclohexyl, each of which is optionally substituted. In some embodiments, the optionally substituted cycloalkyl is

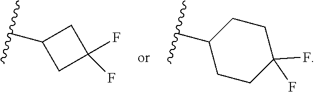 [SMILES: *C1CC(F)(F)C1 ; *C1CCC(F)(F)CC1]

In some embodiments, R⁵ is selected from the group consisting of phenyl, 3-chlorophenyl, 3-chloro-4-fluorophenyl, 3-trifluoromethylphenyl, 3,4-difluorophenyl, and 2,6-difluorophenyl. In some embodiments, R⁵ is cyclopropyl. In some embodiments, R⁵ selected from the group consisting of pyridin-3-yl and 1-methylindazole-6-yl. In some embodiments, R⁵ is selected from the group consisting of H, phenyl, 3-chlorophenyl, 3-chloro-4-fluorophenyl, 3-trifluoromethylphenyl, 3,4-difluorophenyl, cyclopropyl, pyridin-3-yl, 1-methylindazole-6-yl, 3,3-difluorocyclobutyl, and 4,4-difluorocyclohexyl. In some embodiments, R⁵ is 3-chlorophenyl. In some embodiments R⁵ is H. In some embodiments, R⁵ is

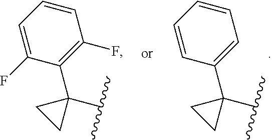 [SMILES: *C1(c2c(F)cccc2F)CC1 ; "or" ; *C1(c2ccccc2)CC1 ; "."]

In some embodiments, R⁵ is —CH₂CH₂Ph. In some embodiments, R⁵ is selected from the group consisting of H, aryl, heteroaryl, alkylenearyl, cycloalkyl, heterocyclyl, alkyl, and haloalkyl, each of which is optionally substituted, or R⁴ and R⁵ together with the atom to which they are attached form an optionally substituted heterocyclyl.

In some embodiments of Formula (I), R⁵ is optionally substituted with one or more halogen, haloalkyl, alkyl, Oalkyl, Ohaloalkyl, cycloalkyl, heterocyclyl aryl, or heteroaryl. In some embodiments, the haloalkyl is selected from CF₃, CHF₂, or CH₂F. In some embodiments, the alkyl is a —C₁₋₅alkyl. In some embodiments, —C₁₋₅alkyl is methyl, ethyl, propyl, i-propyl, butyl, or t-butyl. In some embodiments, the cycloalkyl is a C₃₋₆cycloalkyl. In some embodiments, the aryl is a phenyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the Ohaloalkyl is selected from OCF₃, OCHF₂, or OCH₂F. In some embodiments, the Oalkyl is O-methyl, O-ethyl, O-propyl, O-i-propyl, O-butyl, or O-t-butyl.

In some embodiments of Formula (I), R⁴ is H or —C₁₋₅alkyl and R⁵ is aryl. In some embodiments, R⁴ is H or —C₁₋₅alkyl and R⁵ is heteroaryl. In some embodiments, R⁴ is H or —C₁₋₅alkyl and R⁵ is cycloalkyl. In some embodiments, the —C₁₋₅alkyl is methyl, ethyl, or propyl.

In some embodiments, the —C₁₋₅alkyl is methyl. In some embodiments, the aryl is optionally substituted phenyl. In some embodiments, the heteroaryl is a 5- to 14-membered heteroaryl having 1, 2, or 3 heteroatoms selected from the group consisting of N, O, and S. In some embodiments, the optionally substituted 5- to 14-membered heteroaryl is selected from the group consisting of pyrazolyl, imidazolyl, oxazolyl, thiazolyl, pyridinyl, pyrimidinyl, pyrazinyl, pyridazinyl, quinolinyl, isoquinolinyl, quinoxalinyl, cinnolinyl, indolizinyl, azaindolizinyl, indolyl, azaindolyl, benzoxazolyl, benzthiazolyl, benzfuranyl, benzthiophenyl, imidazopyridinyl, imidazopyrazinyl, and benzimidazolyl. In some embodiments, the heteroaryl is a 5- or 6-membered heteroaryl ring. In some embodiments, the 5-membered heteroaryl is optionally substituted pyrazolyl, imidazolyl, or oxazolyl. In some embodiments, the 6-membered heteroaryl is optionally substituted pyridinyl, pyrimidinyl, pyrazinyl, or pyridazinyl. In some embodiments, cycloalkyl is optionally substituted cyclopropyl, cyclobutyl, cyclopentyl, or cyclohexyl. In some embodiments, aryl is optionally substituted with one or more substituents selected from the group consisting of halogen, C₁₋₆haloalkyl, C₁₋₆alkyl, O-C₁₋₆alkyl, O—C₁₋₆haloalkyl, or C₃₋₆cycloalky. In some embodiments, heteroaryl is optionally substituted with one or more substituents selected from the group consisting of halogen, C₁₋₆haloalkyl, C₁₋₆alkyl, O—C₁₋₆alkyl, O—C₁₋₆haloalkyl, or C₃₋₆cycloalky.

In some embodiments of Formula (I), R⁴ is —(CO)R² and R⁵ is aryl. In some embodiments, R⁴ is —(CO)R² and R⁵ is heteroaryl. In some embodiments, R⁴ is —(CO)R² and R⁵ is cycloalkyl. In some embodiments, the aryl is optionally substituted phenyl. In some embodiments, the aryl is optionally substituted phenyl. In some embodiments, the heteroaryl is a 5- to 14-membered heteroaryl having 1, 2, or 3 heteroatoms selected from the group consisting of N, O, and S. In some embodiments, the optionally substituted 5- to 14-membered heteroaryl is selected from the group consisting of pyrazolyl, imidazolyl, oxazolyl, thiazolyl, pyridinyl, pyrimidinyl, pyrazinyl, pyridazinyl, quinolinyl, isoquinolinyl, quinoxalinyl, cinnolinyl, indolizinyl, azaindolizinyl, indolyl, azaindolyl, benzoxazolyl, benzthiazolyl, benzfuranyl, benzthiophenyl, imidazopyridinyl, imidazopyrazinyl, and benzimidazolyl. In some embodiments, the heteroaryl is a 5- or 6-membered heteroaryl ring. In some embodiments, the 5-membered heteroaryl is optionally substituted pyrazolyl, imidazolyl, oxazolyl, In some embodiments, the 6-membered heteroaryl is optionally substituted pyridinyl, pyrimidinyl, pyrazinyl, or pyridazinyl. In some embodiments, cycloalkyl is optionally substituted cyclopropyl, cyclobutyl, cyclopentyl, or cyclohexyl. In some embodiments, aryl is optionally substituted with one or more substituents selected from the group consisting of halogen, C₁₋₆haloalkyl, C₁₋₆alkyl, O—C₁₋₆alkyl, O—C₁₋₆haloalkyl, or C₃₋₆cycloalky. In some embodiments, heteroaryl is optionally substituted with one or more substituents selected from the group consisting of halogen, C₁₋₆haloalkyl, C₁₋₆alkyl, O—C₁₋₆alkyl, O—C₁₋₆haloalkyl, or C₃₋₆cycloalky.

In some embodiments of Formula (I), R⁴ is —(SO₂)R² and R⁵ is aryl. In some embodiments, R⁴ is —(SO₂)R² and R⁵ is heteroaryl. In some embodiments, R⁴ is —(SO₂)R² and R⁵ is cycloalkyl. In some embodiments, the aryl is optionally substituted phenyl. In some embodiments, the heteroaryl is a 5- to 14-membered heteroaryl having 1, 2, or 3 heteroatoms selected from the group consisting of N, O, and S. In some embodiments, the optionally substituted 5- to 14-membered heteroaryl is selected from the group consisting of pyrazolyl, imidazolyl, oxazolyl, thiazolyl, pyridinyl, pyrimidinyl, pyrazinyl, pyridazinyl, quinolinyl, isoquinolinyl, quinoxalinyl, cinnolinyl, indolizinyl, azaindolizinyl, indolyl, azaindolyl, benzoxazolyl, benzthiazolyl, benzfuranyl, benzthiophenyl, imidazopyridinyl, imidazopyrazinyl, and benzimidazolyl. In some embodiments, the heteroaryl is a 5- or 6-membered heteroaryl ring. In some embodiments, the 5-membered heteroaryl is optionally substituted pyrazolyl, imidazolyl, or oxazolyl. In some embodiments, the 6-membered heteroaryl is optionally substituted pyridinyl, pyrimidinyl, pyrazinyl, or pyridazinyl. In some embodiments, cycloalkyl is optionally substituted cyclopropyl, cycloybutyl, cyclopentyl, or cyclohexyl. In some embodiments, aryl is optionally substituted with one or more substituents selected from the group consisting of halogen, C₁₋₆haloalkyl, C₁₋₆alkyl, O—C₁₋₆alkyl, 0-C₁₋₆haloalkyl, or C₃₋₆cycloalkyl. In some embodiments, heteroaryl is optionally substituted with one or more substituents selected from the group consisting of halogen, C₁₋₆haloalkyl, C₁₋₆alkyl, O—C₁₋₆alkyl, O—C₁₋₆haloalkyl, or C₃₋₆cycloalkyl. In some embodiments, the C₁₋₆haloalkyl is CF₃, CHF₂, or CH₂F. In some embodiments, the O—C₁₋₆haloalkyl is OCF₃, OCHF₂, or OCH₂F. In some embodiments, cycloalkyl is optionally substituted with halogen, C₁₋₆alkyl, or O—C₁₋₆alkyl.

In some embodiments of Formula (I), R⁴ and R⁵ together with the atom to which they are attached form a cycloalkyl or heterocyclyl. In some embodiments, R⁴ and R⁵ together with the atom to which they are attached form a cycloalkyl or heterocyclyl, each of which is optionally substituted. In some embodiments, the cycloalkyl or heterocyclyl is optionally substituted with —NS(O₂)(alkyl)(aryl). In some embodiments, the alkyl is C₁₋₅alkyl and the aryl is phenyl optionally substituted with one or more halogen atoms. In some embodiments, the heterocyclyl is a 4- to 10-membered heterocyclyl. In some embodiments the heterocyclyl is a saturated 4- to 7-membered heterocyclyl.

In some embodiments of Formula (I), n is 0 and R⁴ and R⁵ together with the atom to which they are attached form an optionally substituted heterocyclyl selected from the group consisting of:

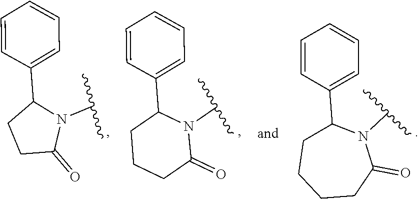

In some embodiments, the optionally substituted heterocyclyl is

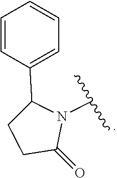 [SMILES: *N1C(=O)CCC1c1ccccc1]

In some embodiments, the optionally substituted heterocyclyl is

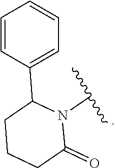 [SMILES: *N1C(=O)CCCC1c1ccccc1]

In some embodiments, the optionally substituted heterocyclyl is

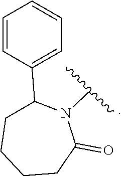 [SMILES: *N1C(=O)CCCCC1c1ccccc1]

In some embodiments of Formula (I) R¹ is selected from the group consisting of

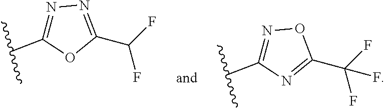 [SMILES: *c1nnc(C(F)F)o1 ; *c1noc(C(F)(F)F)n1]

In some embodiments of Formula (I), R¹ is

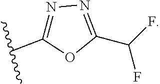 [SMILES: *c1nnc(C(F)F)o1]

In some embodiments, R¹ is

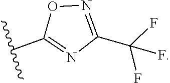 [SMILES: *c1nc(C(F)(F)F)no1]

In some embodiments, R¹ is

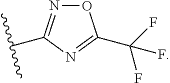 [SMILES: *c1noc(C(F)(F)F)n1]

In some embodiments, R¹

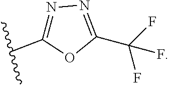 [SMILES: *c1nnc(C(F)(F)F)o1]

In some embodiments of Formula (I), R^{a} is H, halo, C₁₋₃alkyl, or haloalkyl. In some embodiments, R^{a} is H. In some embodiments, R^{a} is C₁₋₃alkyl. In some embodiments, R^{a} is haloalkyl. In some embodiments, halo is F. In some embodiments, the C₁₋₃alkyl alkyl is methyl, ethyl or isopropyl. In some embodiments, haloalkyl is CF₃, CHF₂, or CH₂F.

In some embodiments of Formula (I), Y is CH and R⁴ and R⁵ are H.

In some embodiments of Formula (I), Y is N, R⁴ is H, and R⁵ is ethyl optionally substituted with —N(S(O₂)alkyl)(aryl) or —N(S(O₂)cycloalkyl)(aryl). In some embodiments, alkyl is C₁₋₅alkyl, cycloalkyl is C₃₋₆cycloalkyl, and aryl is phenyl optionally substituted with one or more halogen atoms.

In some embodiments of Formula (I), n is 1, X¹ is O or N, Y is N, R¹ is

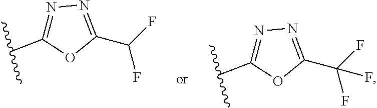 [SMILES: *c1nnc(C(F)F)o1 ; *c1nnc(C(F)(F)F)o1]

R² and R³ are H, R⁴ is H, —C₁₋₅alkyl, —C(O)alkyl, —C(O)cycloalkyl,—(SO₂)NR²R³, —SO₂alkyl, —SO₂haloalkyl and —SO₂cycloalkyl, each of which is optionally substituted, and R⁵ is aryl, heteroaryl, or cycloalkyl, each of which is optionally substituted.

In some embodiments of Formula (I), n is 1, X¹ is O or N, Y is O, R¹ is

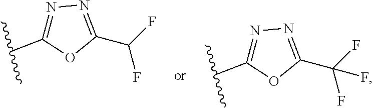 [SMILES: *c1nnc(C(F)F)o1 ; *c1nnc(C(F)(F)F)o1]

R² and R³ are H, and R⁵ is aryl, heteroaryl, cycloalkyl, or alkylenecycloalkyl, each of which is optionally substituted.

In some embodiments of Formula (I), n is 0, X¹ is O or N, Y is N, R¹ is

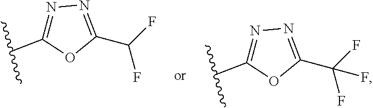 [SMILES: *c1nnc(C(F)F)o1 ; *c1nnc(C(F)(F)F)o1]

and R⁴ and R⁵ taken together with the atom to which they are attached form a cycloalkyl or heterocyclyl, each of which is optionally substituted.

In some embodiments, the present disclosure provides a compound of Formula (Ia) or a salt thereof:

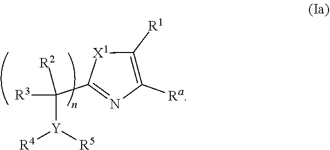

wherein:
R¹, R², R³, R⁴, R⁵, R^{a}, X¹, n, and Y are as defined above for Formula (I).

In some embodiments of Formula (Ia), R¹ is

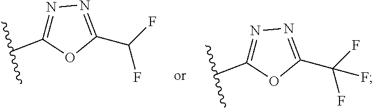 [SMILES: *c1nnc(C(F)F)o1 ; *c1nnc(C(F)(F)F)o1]

n is 1; Y is N; X¹ is S or O; and variables R², R³, R⁴, R⁵, and R^{a} are as defined above for Formula (I).

In some embodiments of Formula (Ia), n is 1, X¹ is S, Y is N, R¹ is

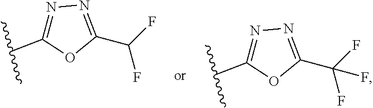 [SMILES: *c1nnc(C(F)F)o1 ; *c1nnc(C(F)(F)F)o1]

R² and R³ are H, R⁴ is —SO₂alkyl, —SO₂haloalkyl, or —SO₂cycloalkyl, each of which is optionally substituted, R⁵ is heteroaryl, each of which is optionally substituted, and R^{a} is H or F. In some further embodiments, R⁴ is —SO₂C₁₋₅alkyl, —SO₂cyclopropyl, —SO₂CF₃ or —SO₂CHF₂, and the heteroaryl is optionally substituted pyridine or pyrazine. In some further embodiments, the heteroaryl is optionally substituted pyridine.

In some embodiments of Formula (Ia), n is 1, X¹ is S, Y is N, R¹ is

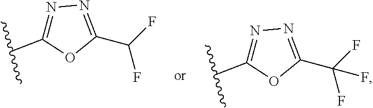 [SMILES: *c1nnc(C(F)F)o1 ; *c1nnc(C(F)(F)F)o1]

R² and R³ are H, R⁴ is —SO₂Me, —SO₂Et, or —SO₂cyclopropyl, each of which is optionally substituted, R⁵ is pyridine or pyrazine, each of which is optionally substituted, and R^{a} is H. In some embodiments, R⁵ is optionally substituted pyridine.

In some embodiments of Formula (Ia), n is 1, X¹ is S, Y is N, R¹ is

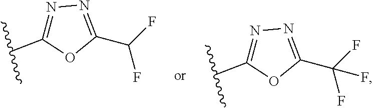 [SMILES: *c1nnc(C(F)F)o1 ; *c1nnc(C(F)(F)F)o1]

R² and R³ are H, R⁴ is —SO₂alkyl or —SO₂cycloalkyl, each of which is optionally substituted, R⁵ is

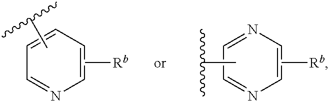

wherein R^{b} is as defined herein. In some embodiments, R^{b} is selected from the group consisting of H, halogen, —C₁₋₅alkyl, haloalkyl, —OC₁₋₅alkyl, —Ohaloalkyl, —CH₂Ohaloalkyl, cyclopropyl, and CN, and R^{a} is H. In some embodiments, the halogen is F or C₁. In some embodiments, the haloalkyl is CF₃, CHF₂, CH₂CF₃, or CF₂CH₃. In some embodiments, the —C₁₋₅alkyl is methyl.

In some embodiments of Formula (Ia), n is 1, X¹ is S, Y is N, R¹ is

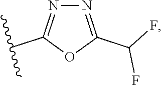 [SMILES: *c1nnc(C(F)F)o1]

R² and R³ are H, R⁴ is —SO₂Me, —SO₂Et, or —SO₂cyclopropyl, each of which is optionally substituted, and R⁵ is

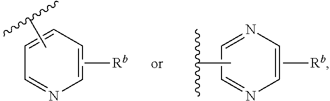

wherein R^{b} is as defined herein. In some embodiments, R^{b} is selected from the group consisting of halogen, —C₁₋₅alkyl, haloalkyl, —OC₁₋₅alkyl, —Ohaloalkyl, —CH₂Ohaloalkyl, cyclopropyl, or CN, and R^{a} is H. In some embodiments, the halogen is F or C₁. In some embodiments, the haloalkyl is CF₃, CHF₂, CH₂CF₃, or CF₂CH₃. In some embodiments, the —C₁₋₅alkyl is methyl.

In some embodiments of Formula (Ia), n is 1, X¹ is S, Y is N, R¹ is

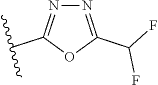 [SMILES: *c1nnc(C(F)F)o1]

R² and R³ are H, R⁴ is —SO₂Me, —SO₂Et, or —SO₂cyclopropyl, each of which is optionally substituted, and R⁵ is

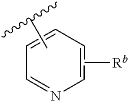

wherein R^{b} is as defined herein. In some embodiments, R^{b} is selected from the group consisting of H, Cl, F, Me, cyclopropyl, CF₃, CHF₂, CF₂CH₃, OCF₃, OCHF₂, OCH₂CF₂H and CN, and R^{a} is H.

In some embodiments, the present disclosure provides a compound of Formula (Ib) or a salt thereof:

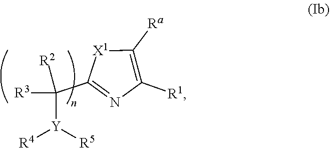

wherein:
R¹, R², R³, R⁴, R⁵, R^{a}, X¹, n, and Y are as defined above for Formula (I).

In some embodiments of Formulas (I)—(Ib), each optionally substituted alkyl is independently an optionally substituted C₁₋₆ alkyl. In some embodiments, the C₁₋₆ alkyl is Me or Et.

In some embodiments of Formulas (I)—(Ib), each optionally substituted haloalkyl is independently an optionally substituted C₁₋₆ haloalkyl. In some embodiments, the C₁₋₆ haloalkyl is CF₃, CHF₂, or CH₂F. In some embodiments, the C₁₋₆ haloalkyl is CF₃ or CHF₂.

In some embodiments of Formulas (I)—(Ib), each optionally substituted cycloalkyl is independently an optionally substituted C₃₋₁₂ cycloalkyl. In some embodiments, the cycloalkyl is a C₃₋₆ cycloalkyl. In some embodiments, the cycloalkyl is selected from the group consisting of cyclopropyl, cyclobutyl, cyclopentyl, and cyclohexyl.

In some embodiments of Formulas (I)—(Ib), each optionally substituted heterocyclyl is independently an optionally substituted 3-12 membered heterocycloalkyl having 1 or 2 heteroatoms independently selected from N, O, and S. In some embodiments, each optionally substituted heterocyclyl is independently an optionally substituted 3-6 membered heterocycloalkyl having 1 or 2 heteroatoms independently selected from N, O, and S. In further embodiments, the heterocycloalkyl is an optionally substituted 5-membered or 6-membered heterocycle having 1 or 2 heteroatoms independently selected from N, O, and S. In some embodiments, the heterocyclyl is selected from the group consisting of aziridinyl, azetidinyl, pyrrolidinyl, piperidinyl, and morpholinyl, and thiomorpholinyl.

In some embodiments of Formulas (I)—(Ib), each optionally substituted aryl is independently a C₆₋₁₂ aryl. In further embodiments, the C₆₋₁₂ aryl is an optionally substituted phenyl.

In some embodiments of Formulas (I)—(Ib), each optionally substituted heteroaryl is independently a 5-12 membered heteroaryl having 1, 2, or 3 heteroatoms independently selected from N, O, and S. In some embodiments, each optionally substituted heteroaryl is independently a 5-12 membered heteroaryl having 3 heteroatoms independently selected from N, O, and S. In some embodiments, each optionally substituted heteroaryl is independently a 5-12 membered heteroaryl having 2 heteroatoms independently selected from N, O, and S. In some embodiments, each optionally substituted heteroaryl is independently a 5-12 membered heteroaryl having 1 heteroatom independently selected from N, O, and S. In further embodiments, each optionally substituted heteroaryl is an optionally substituted 5-membered or 6-membered heteroaryl having 1 heteroatom independently from N, O, and S. In some embodiments, each heteroaryl is independently selected from the group consisting of tetrazole, oxadiazole, thiadiazole, imidazole, pyrazole, thiazole, or oxazole, each of which is optionally substituted.

In some embodiments, the compound of Formula (I) is selected from the group consisting of:

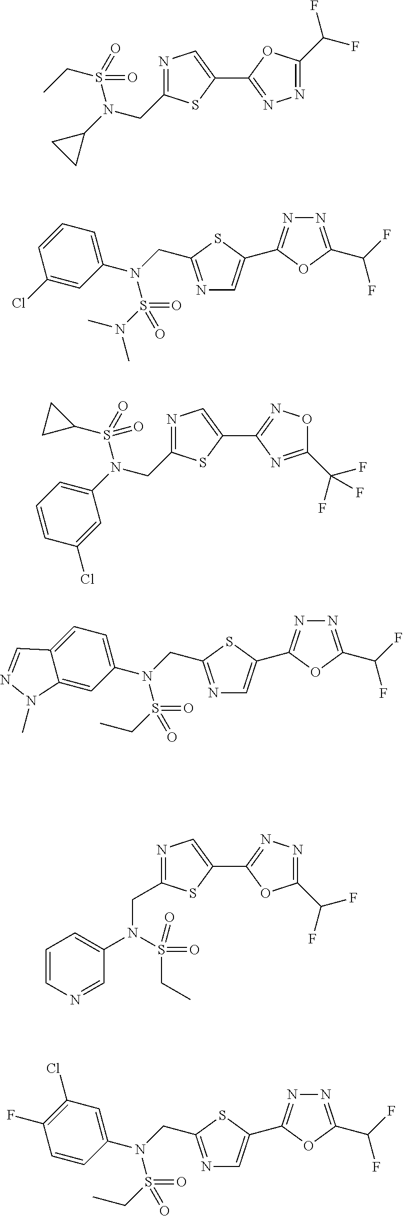

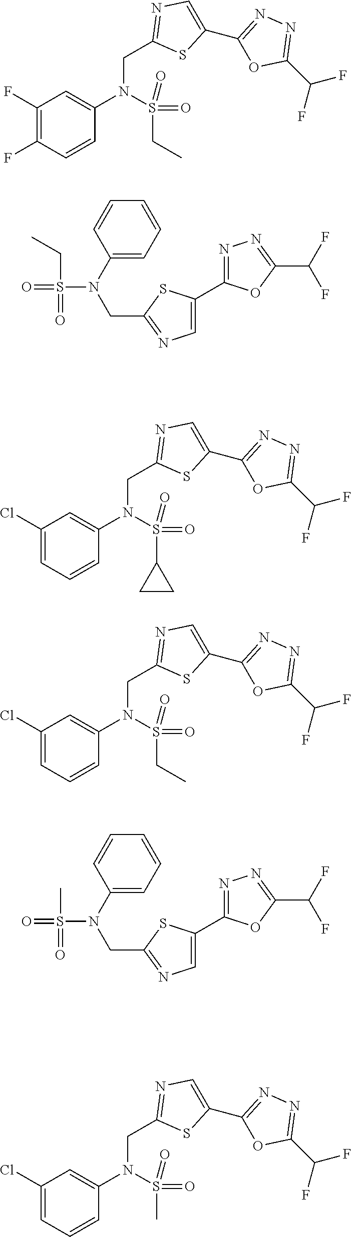

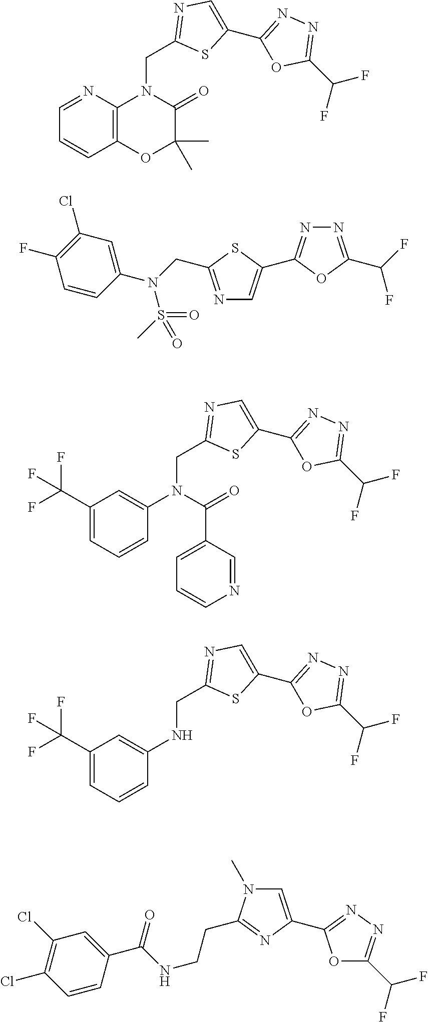

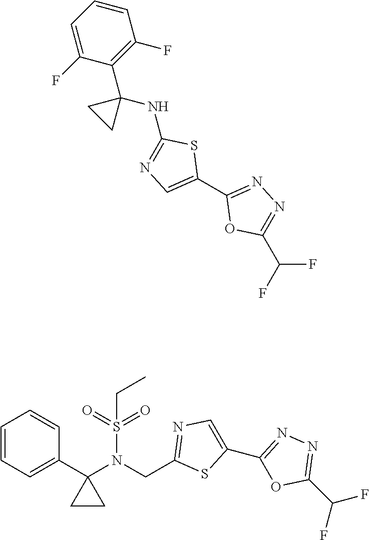 [SMILES: Fc1cccc(F)c1C1(Nc2ncc(-c3nnc(C(F)F)o3)s2)CC1 ; CCS(=O)(=O)N(Cc1ncc(-c2nnc(C(F)F)o2)s1)C1(c2ccccc2)CC1]

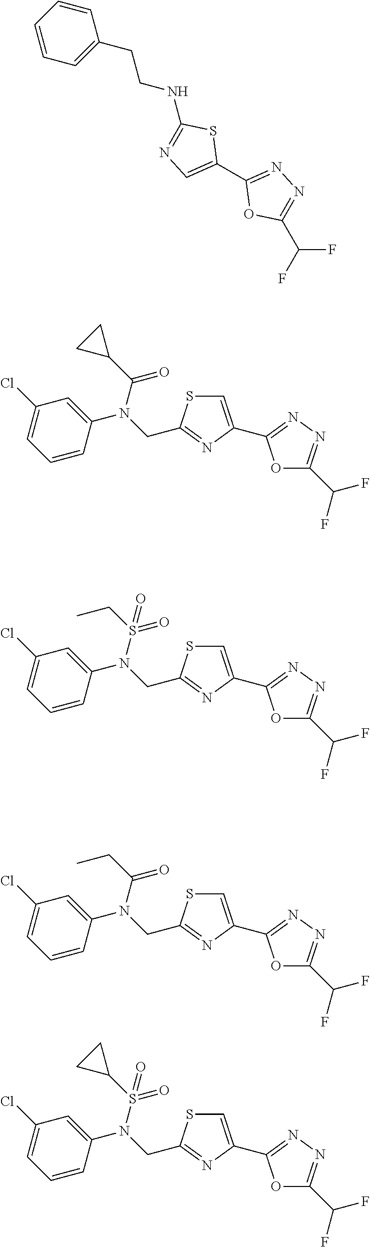 [SMILES: FC(F)c1nnc(-c2cnc(NCCc3ccccc3)s2)o1 ; O=C(C1CC1)N(Cc1nc(-c2nnc(C(F)F)o2)cs1)c1cccc(Cl)c1 ; CCS(=O)(=O)N(Cc1nc(-c2nnc(C(F)F)o2)cs1)c1cccc(Cl)c1 ; CCC(=O)N(Cc1nc(-c2nnc(C(F)F)o2)cs1)c1cccc(Cl)c1 ; O=S(=O)(C1CC1)N(Cc1nc(-c2nnc(C(F)F)o2)cs1)c1cccc(Cl)c1]

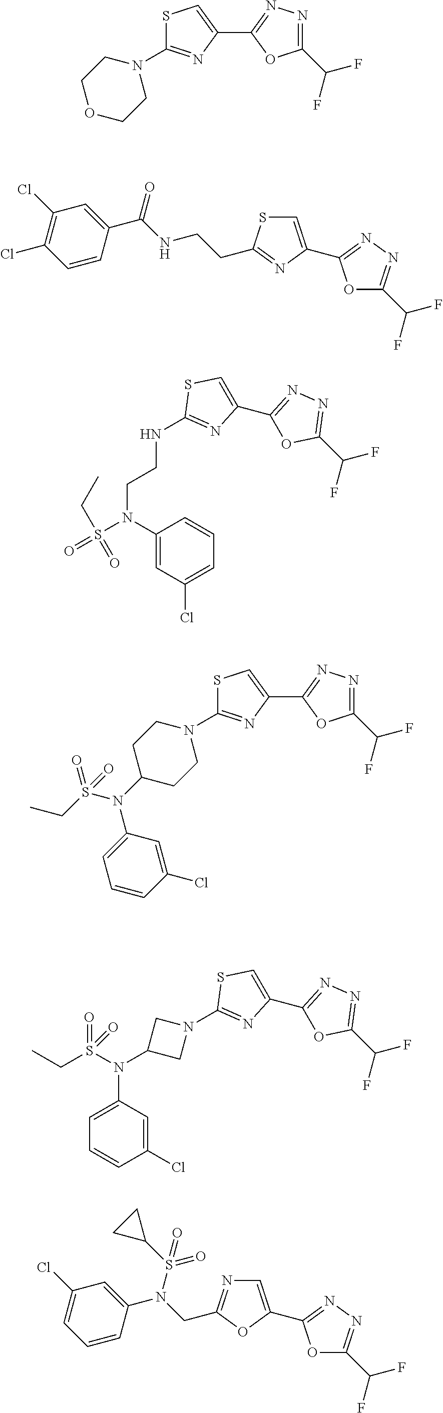

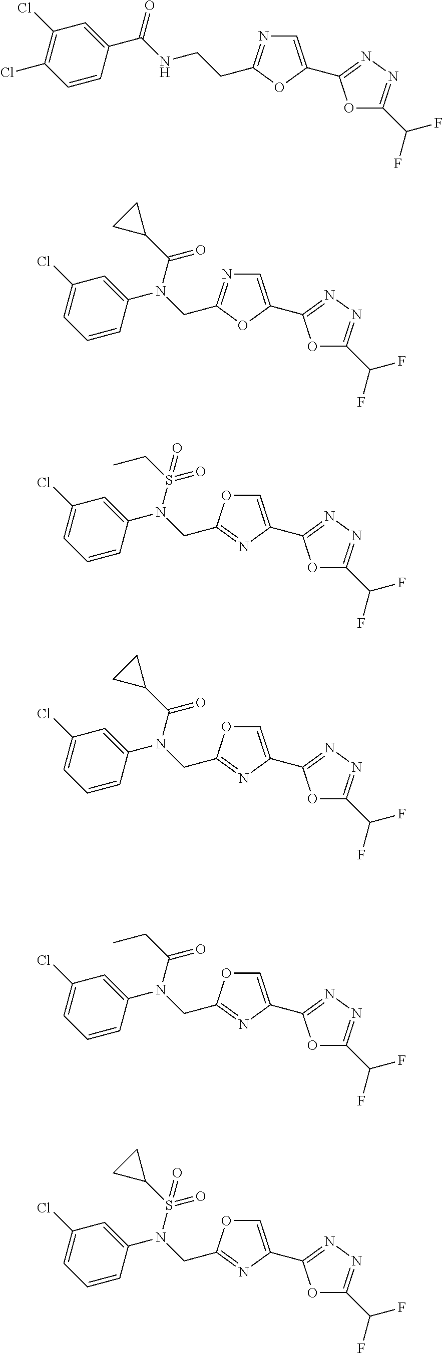 [SMILES: O=C(NCCc1ncc(-c2nnc(C(F)F)o2)o1)c1ccc(Cl)c(Cl)c1 ; O=C(C1CC1)N(Cc1ncc(-c2nnc(C(F)F)o2)o1)c1cccc(Cl)c1 ; CCS(=O)(=O)N(Cc1nc(-c2nnc(C(F)F)o2)co1)c1cccc(Cl)c1 ; O=C(C1CC1)N(Cc1nc(-c2nnc(C(F)F)o2)co1)c1cccc(Cl)c1 ; CCC(=O)N(Cc1nc(-c2nnc(C(F)F)o2)co1)c1cccc(Cl)c1 ; O=S(=O)(C1CC1)N(Cc1nc(-c2nnc(C(F)F)o2)co1)c1cccc(Cl)c1]

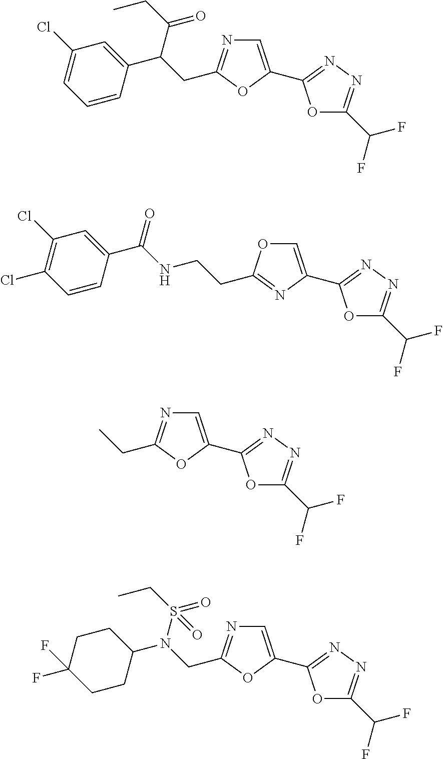 [SMILES: CCC(=O)C(Cc1ncc(-c2nnc(C(F)F)o2)o1)c1cccc(Cl)c1 ; O=C(NCCc1nc(-c2nnc(C(F)F)o2)co1)c1ccc(Cl)c(Cl)c1 ; CCc1ncc(-c2nnc(C(F)F)o2)o1 ; CCS(=O)(=O)N(Cc1ncc(-c2nnc(C(F)F)o2)o1)C1CCC(F)(F)CC1]

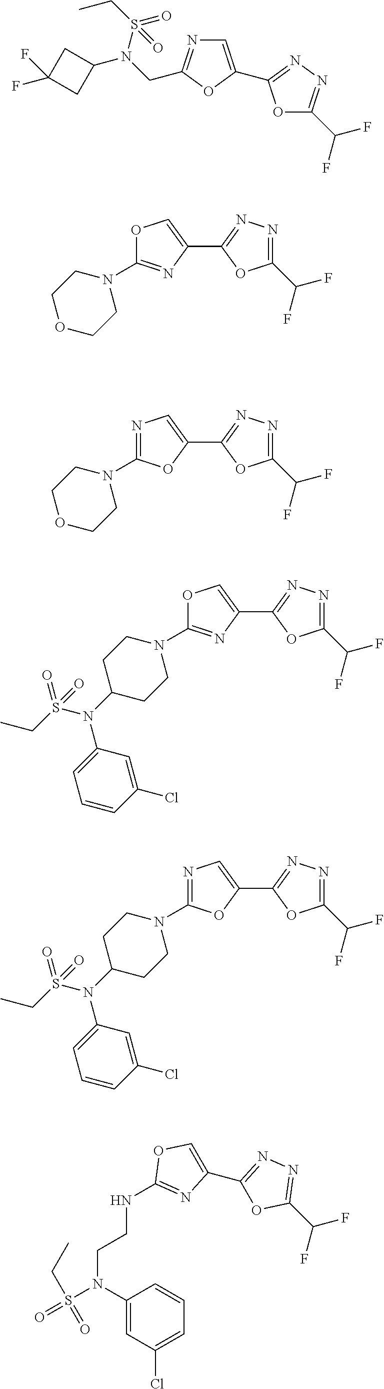 [SMILES: CCS(=O)(=O)N(Cc1ncc(-c2nnc(C(F)F)o2)o1)C1CC(F)(F)C1 ; FC(F)c1nnc(-c2coc(N3CCOCC3)n2)o1 ; FC(F)c1nnc(-c2cnc(N3CCOCC3)o2)o1 ; CCS(=O)(=O)N(c1cccc(Cl)c1)C1CCN(c2nc(-c3nnc(C(F)F)o3)co2)CC1 ; CCS(=O)(=O)N(c1cccc(Cl)c1)C1CCN(c2ncc(-c3nnc(C(F)F)o3)o2)CC1 ; CCS(=O)(=O)N(CCNc1nc(-c2nnc(C(F)F)o2)co1)c1cccc(Cl)c1]

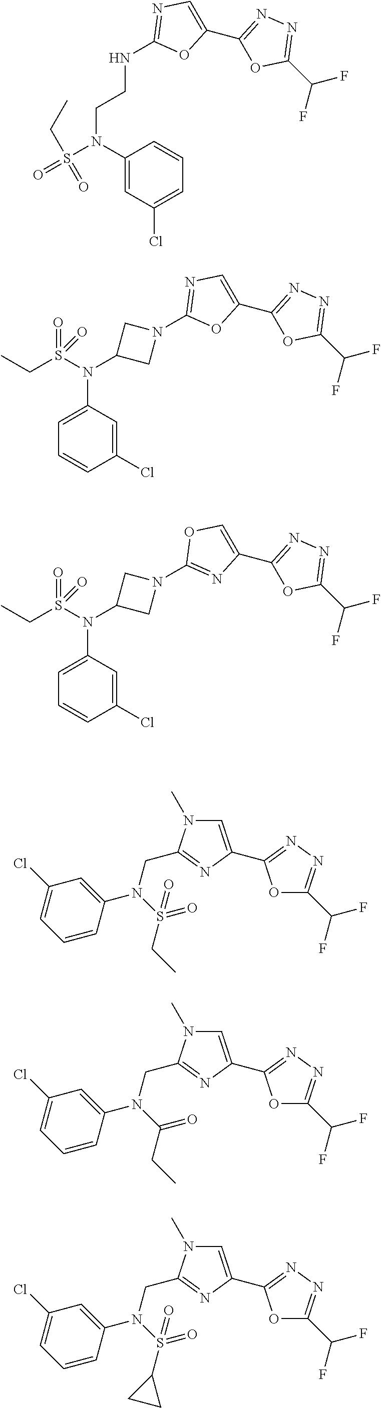

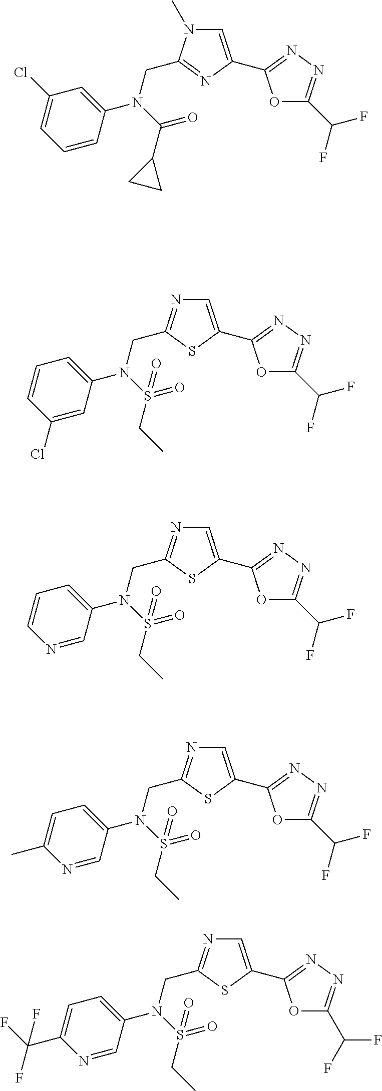 [SMILES: Cn1cc(-c2nnc(C(F)F)o2)nc1CN(C(=O)C1CC1)c1cccc(Cl)c1 ; CCS(=O)(=O)N(Cc1ncc(-c2nnc(C(F)F)o2)s1)c1cccc(Cl)c1 ; CCS(=O)(=O)N(Cc1ncc(-c2nnc(C(F)F)o2)s1)c1cccnc1 ; CCS(=O)(=O)N(Cc1ncc(-c2nnc(C(F)F)o2)s1)c1ccc(C)nc1 ; CCS(=O)(=O)N(Cc1ncc(-c2nnc(C(F)F)o2)s1)c1ccc(C(F)(F)F)nc1]

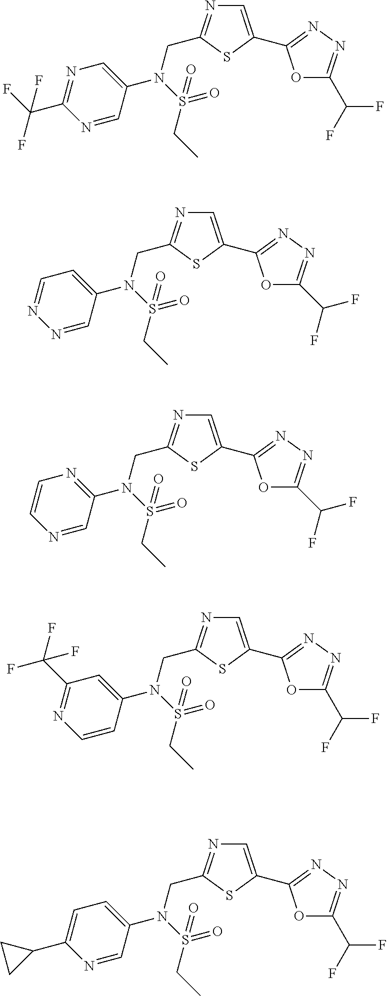 [SMILES: CCS(=O)(=O)N(Cc1ncc(-c2nnc(C(F)F)o2)s1)c1cnc(C(F)(F)F)nc1 ; CCS(=O)(=O)N(Cc1ncc(-c2nnc(C(F)F)o2)s1)c1ccnnc1 ; CCS(=O)(=O)N(Cc1ncc(-c2nnc(C(F)F)o2)s1)c1cnccn1 ; CCS(=O)(=O)N(Cc1ncc(-c2nnc(C(F)F)o2)s1)c1ccnc(C(F)(F)F)c1 ; CCS(=O)(=O)N(Cc1ncc(-c2nnc(C(F)F)o2)s1)c1ccc(C2CC2)nc1]

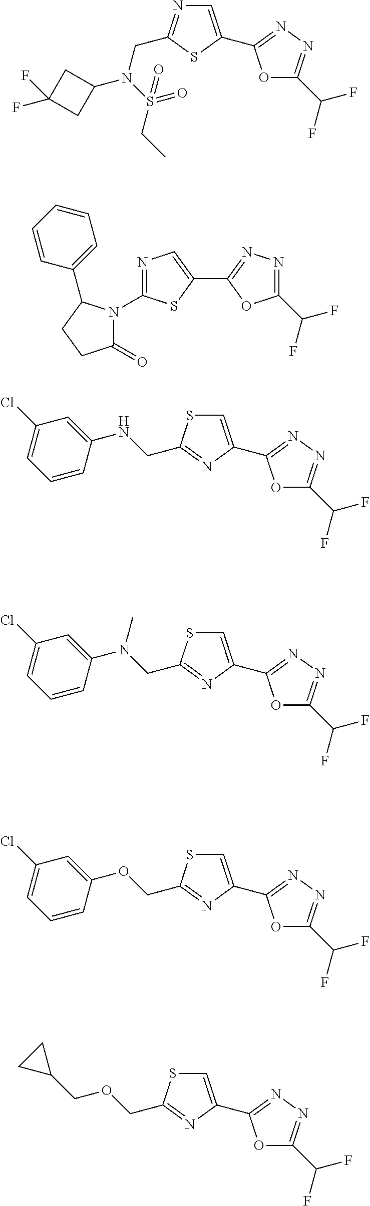 [SMILES: CCS(=O)(=O)N(Cc1ncc(-c2nnc(C(F)F)o2)s1)C1CC(F)(F)C1 ; O=C1CCC(c2ccccc2)N1c1ncc(-c2nnc(C(F)F)o2)s1 ; FC(F)c1nnc(-c2csc(CNc3cccc(Cl)c3)n2)o1 ; CN(Cc1nc(-c2nnc(C(F)F)o2)cs1)c1cccc(Cl)c1 ; FC(F)c1nnc(-c2csc(COc3cccc(Cl)c3)n2)o1 ; FC(F)c1nnc(-c2csc(COCC3CC3)n2)o1]

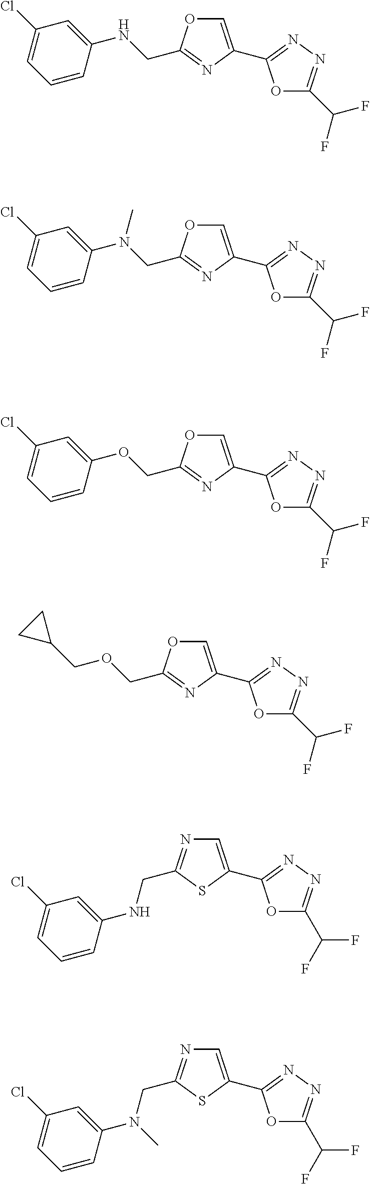 [SMILES: FC(F)c1nnc(-c2coc(CNc3cccc(Cl)c3)n2)o1 ; CN(Cc1nc(-c2nnc(C(F)F)o2)co1)c1cccc(Cl)c1 ; FC(F)c1nnc(-c2coc(COc3cccc(Cl)c3)n2)o1 ; FC(F)c1nnc(-c2coc(COCC3CC3)n2)o1 ; FC(F)c1nnc(-c2cnc(CNc3cccc(Cl)c3)s2)o1 ; CN(Cc1ncc(-c2nnc(C(F)F)o2)s1)c1cccc(Cl)c1]

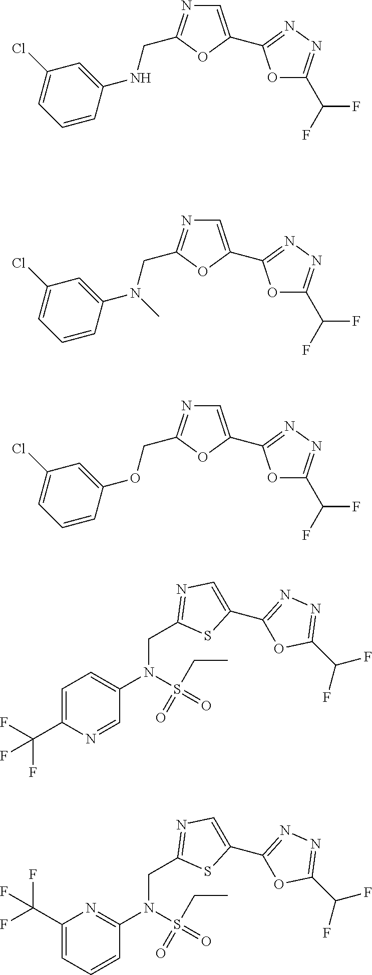 [SMILES: FC(F)c1nnc(-c2cnc(CNc3cccc(Cl)c3)o2)o1 ; CN(Cc1ncc(-c2nnc(C(F)F)o2)o1)c1cccc(Cl)c1 ; FC(F)c1nnc(-c2cnc(COc3cccc(Cl)c3)o2)o1 ; CCS(=O)(=O)N(Cc1ncc(-c2nnc(C(F)F)o2)s1)c1ccc(C(F)(F)F)nc1 ; CCS(=O)(=O)N(Cc1ncc(-c2nnc(C(F)F)o2)s1)c1cccc(C(F)(F)F)n1]

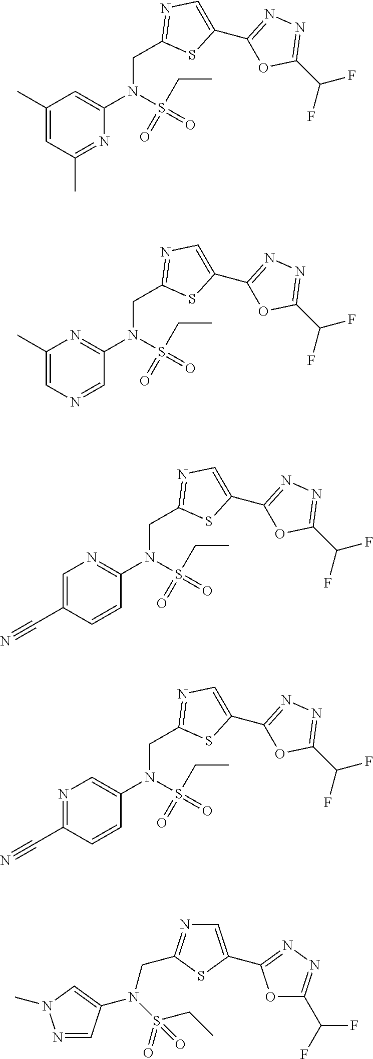 [SMILES: CCS(=O)(=O)N(Cc1ncc(-c2nnc(C(F)F)o2)s1)c1cc(C)cc(C)n1 ; CCS(=O)(=O)N(Cc1ncc(-c2nnc(C(F)F)o2)s1)c1cncc(C)n1 ; CCS(=O)(=O)N(Cc1ncc(-c2nnc(C(F)F)o2)s1)c1ccc(C#N)cn1 ; CCS(=O)(=O)N(Cc1ncc(-c2nnc(C(F)F)o2)s1)c1ccc(C#N)nc1 ; CCS(=O)(=O)N(Cc1ncc(-c2nnc(C(F)F)o2)s1)c1cnn(C)c1]

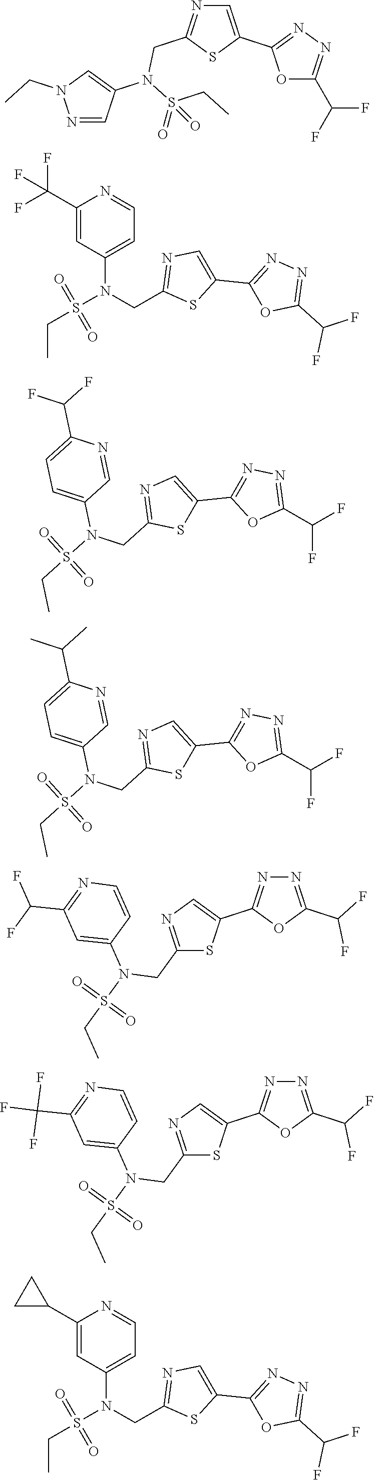

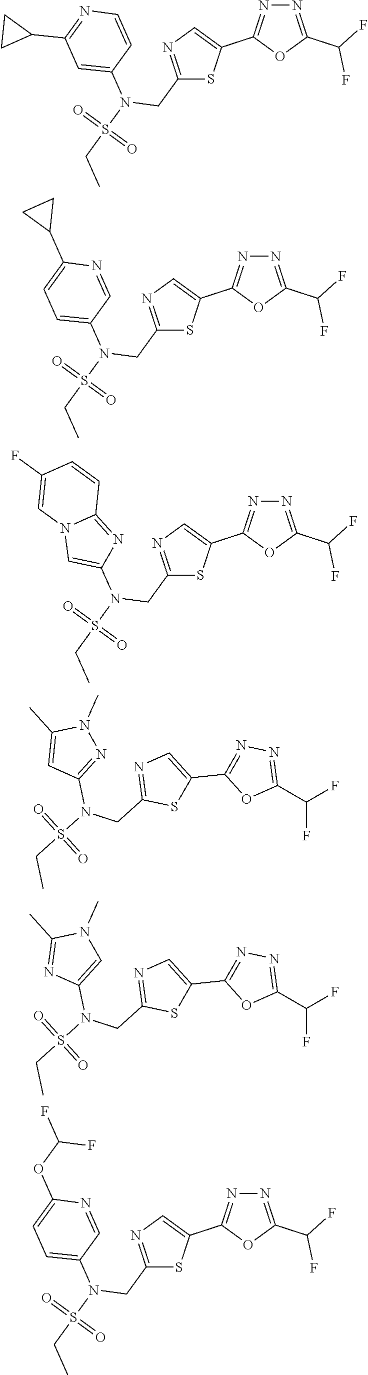

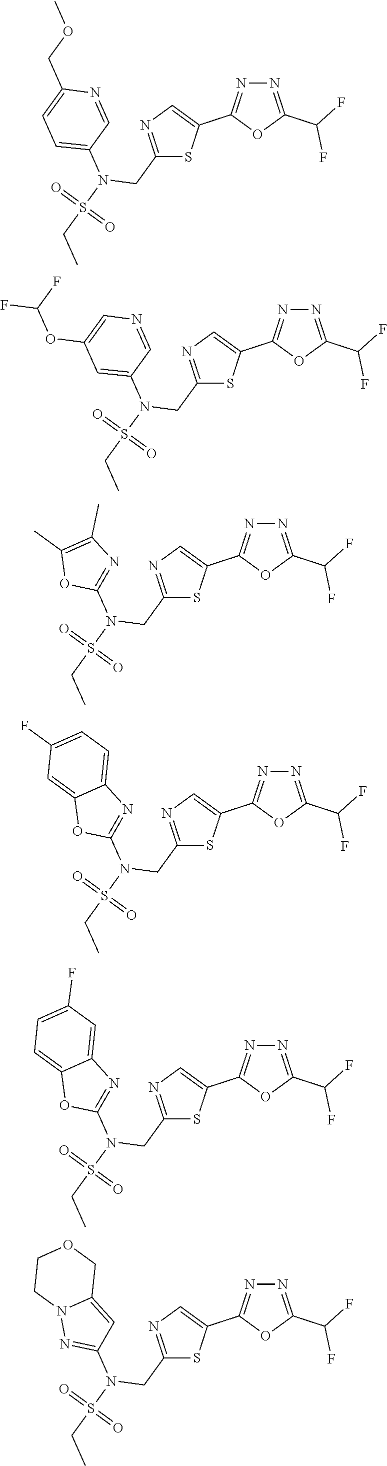

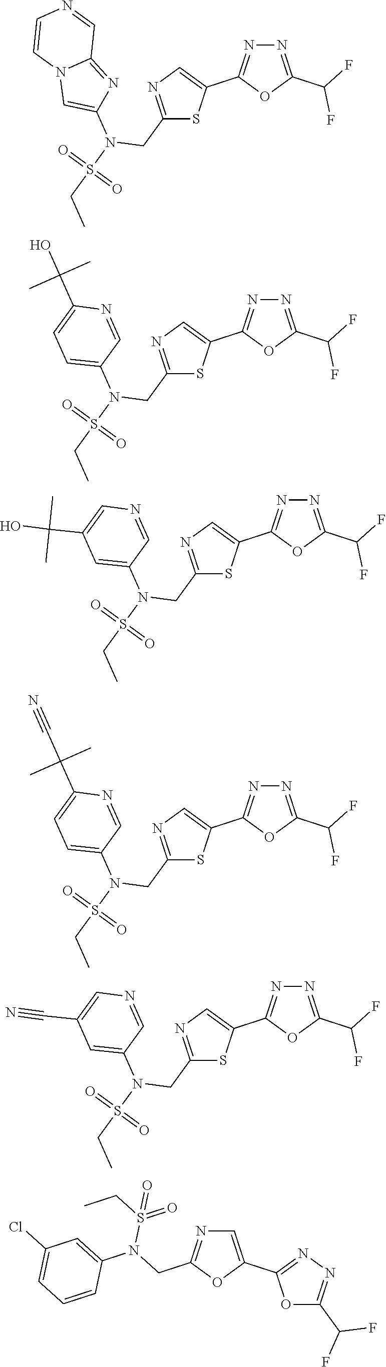

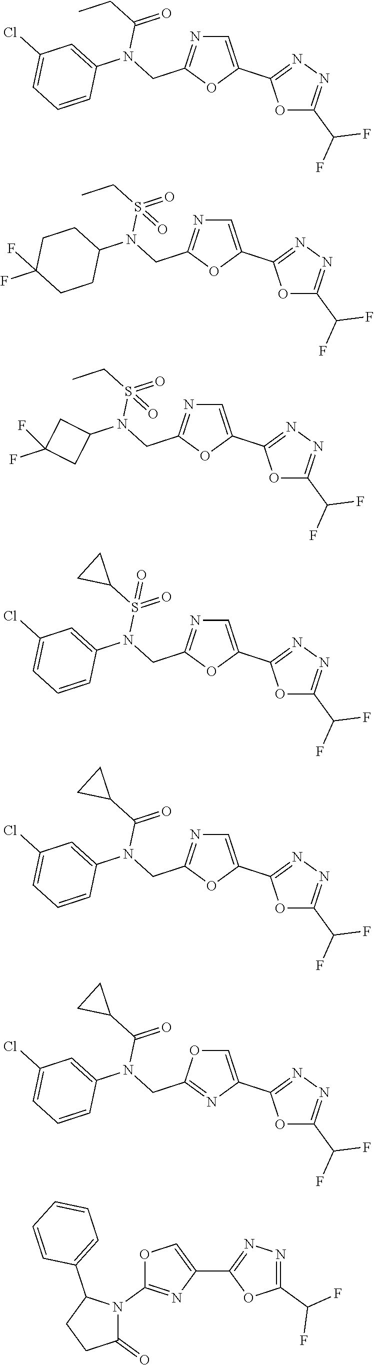

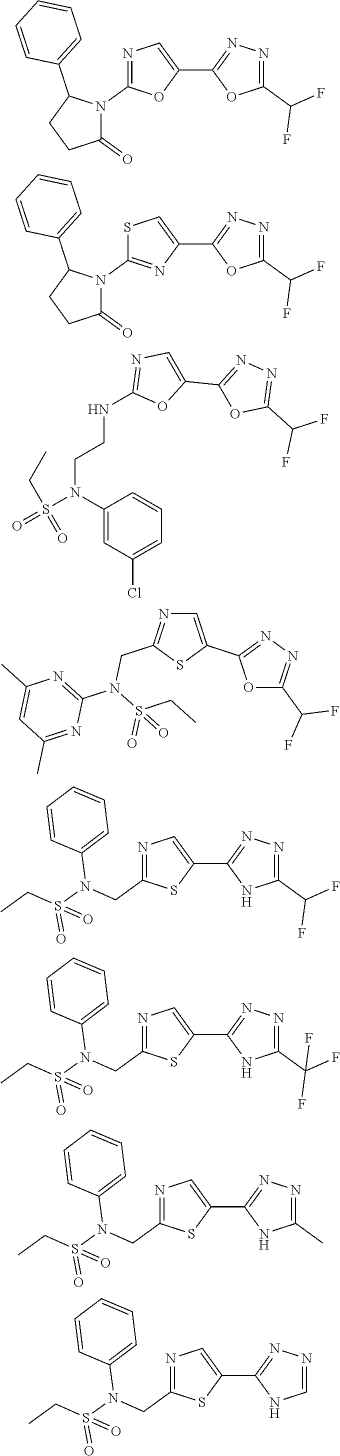

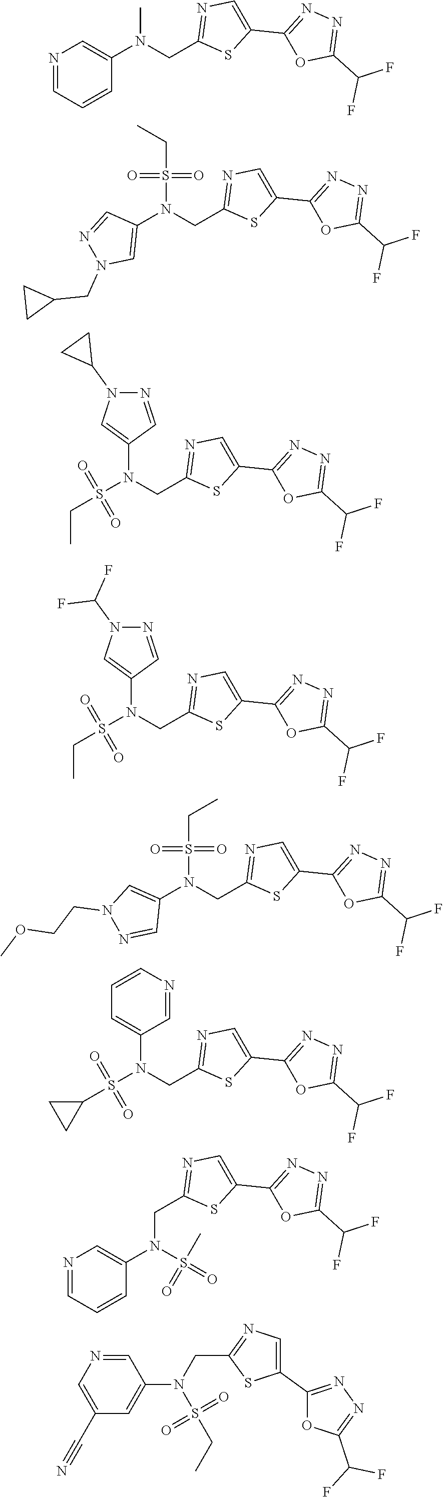

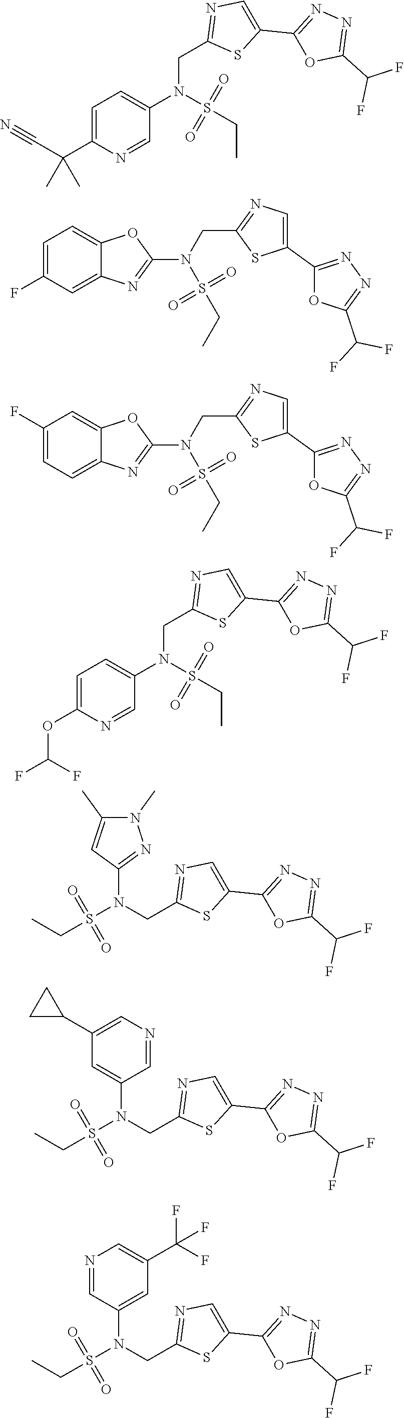

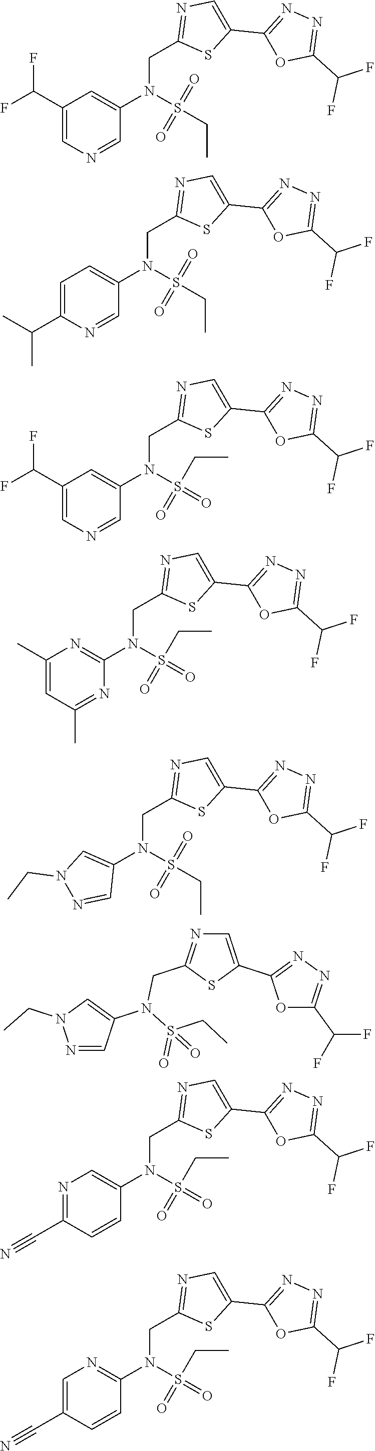

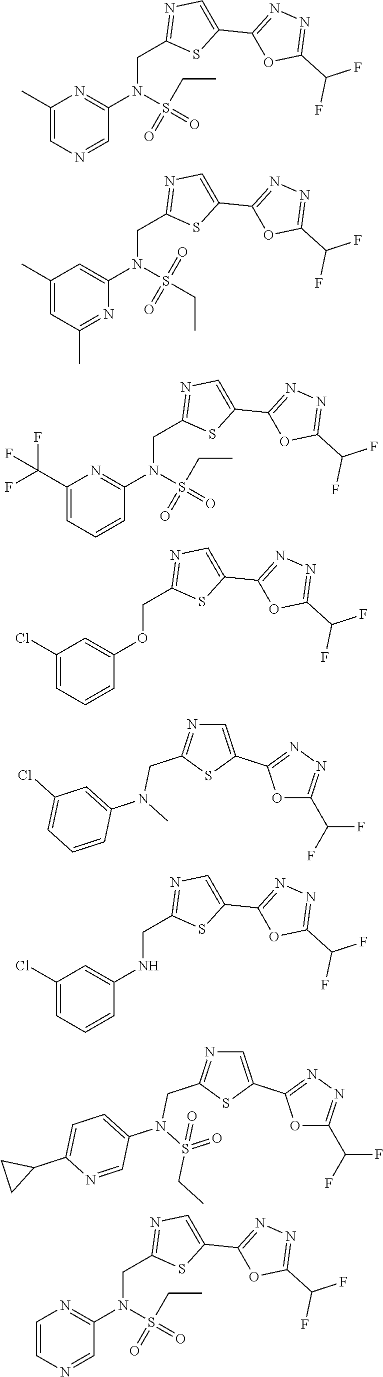

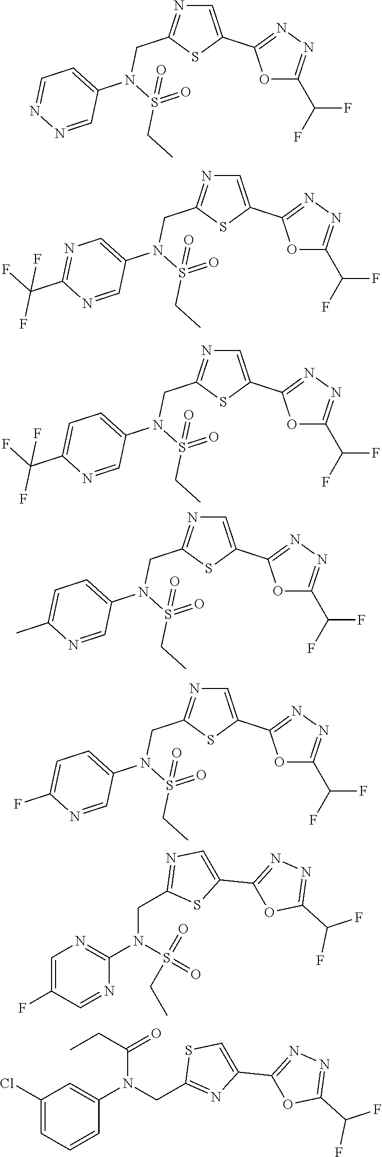

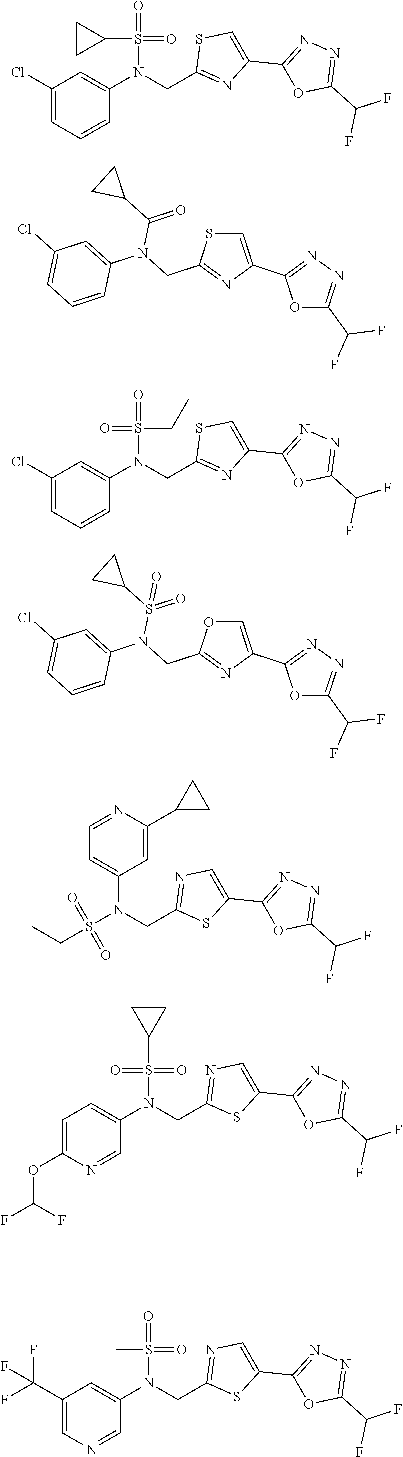

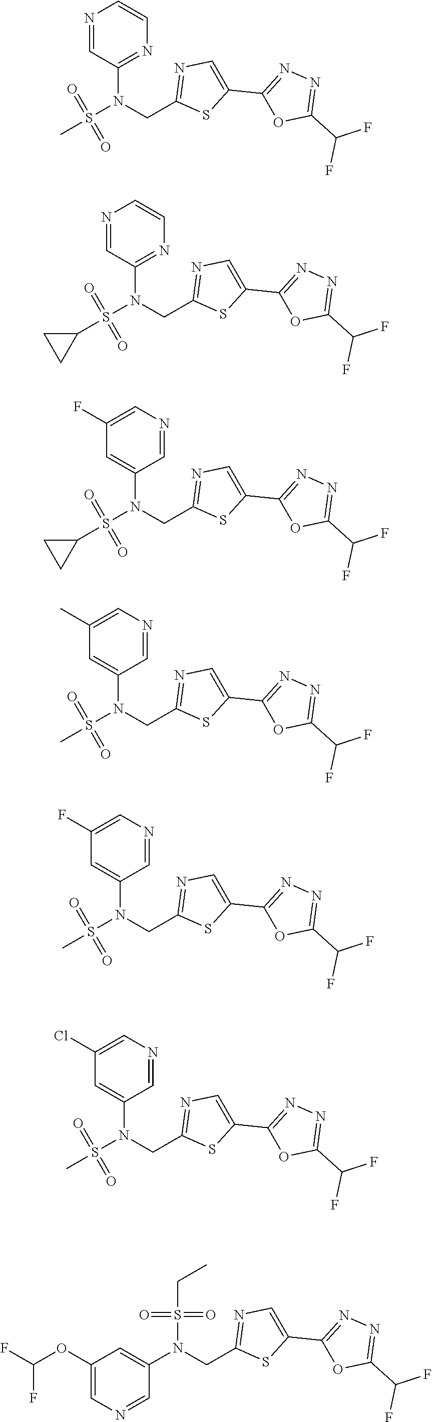

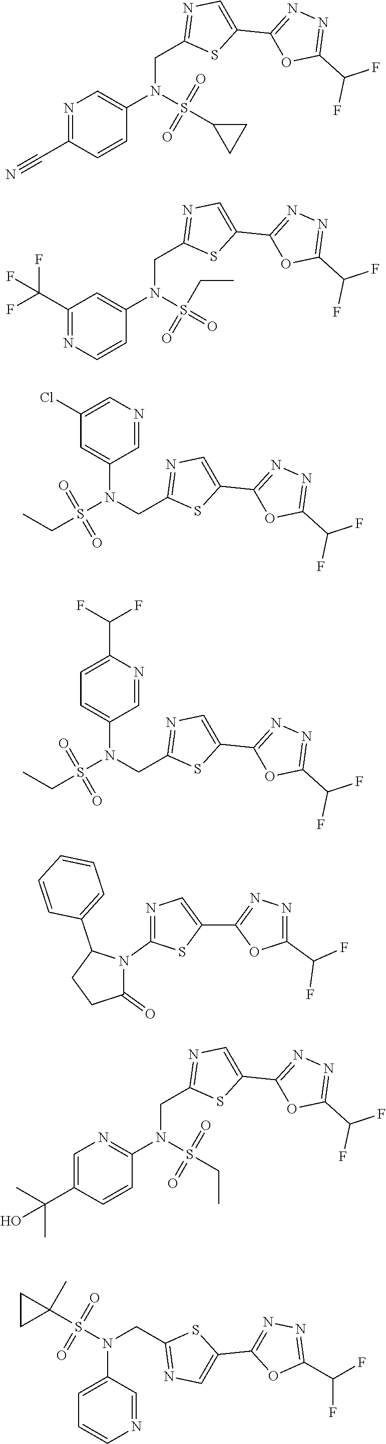

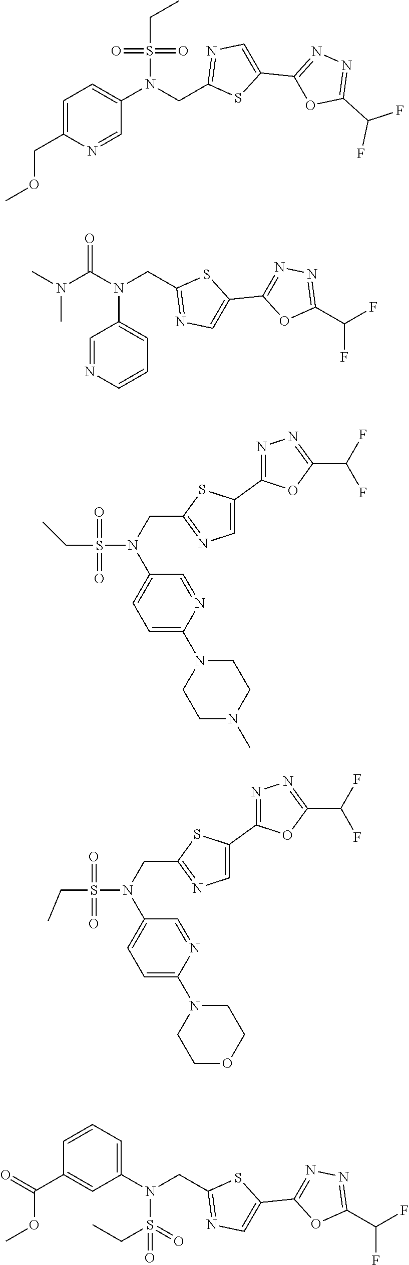

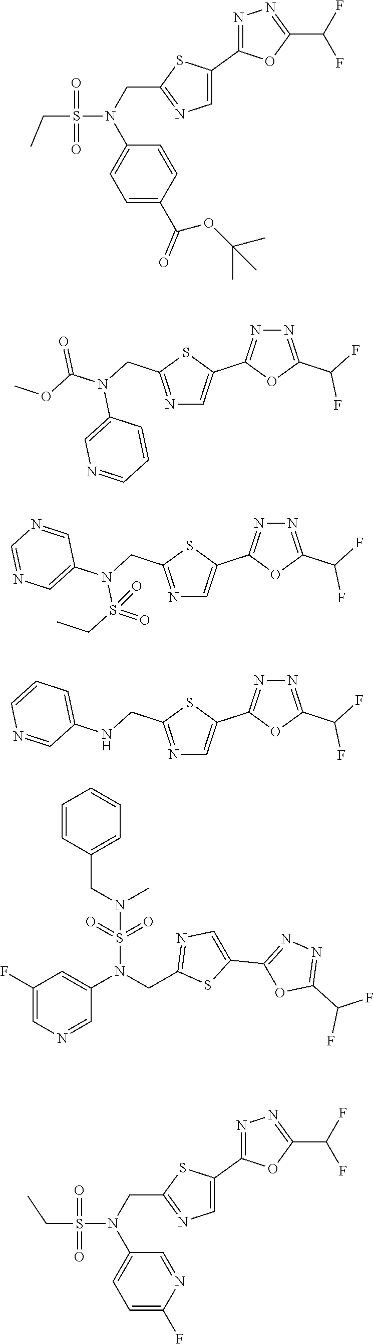 [SMILES: CCS(=O)(=O)N(Cc1ncc(-c2nnc(C(F)F)o2)s1)c1ccc(C(=O)OC(C)(C)C)cc1 ; COC(=O)N(Cc1ncc(-c2nnc(C(F)F)o2)s1)c1cccnc1 ; CCS(=O)(=O)N(Cc1ncc(-c2nnc(C(F)F)o2)s1)c1cncnc1 ; FC(F)c1nnc(-c2cnc(CNc3cccnc3)s2)o1 ; CN(Cc1ccccc1)S(=O)(=O)N(Cc1ncc(-c2nnc(C(F)F)o2)s1)c1cncc(F)c1 ; CCS(=O)(=O)N(Cc1ncc(-c2nnc(C(F)F)o2)s1)c1ccc(F)nc1]

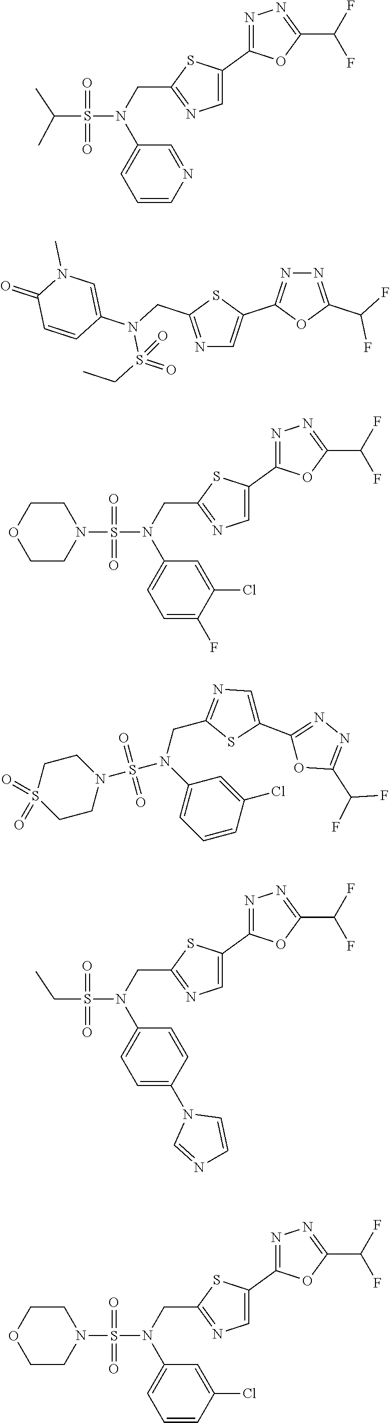

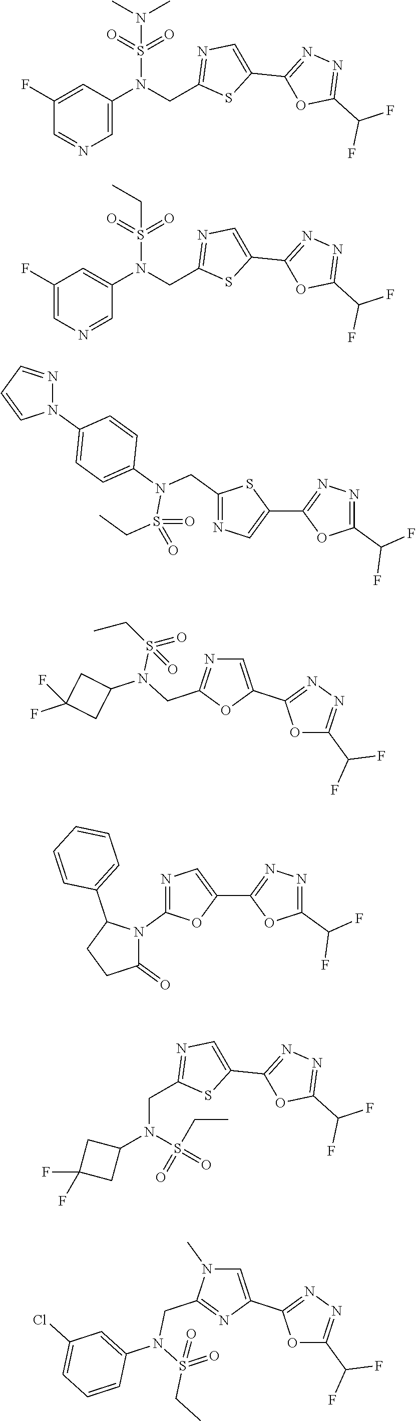

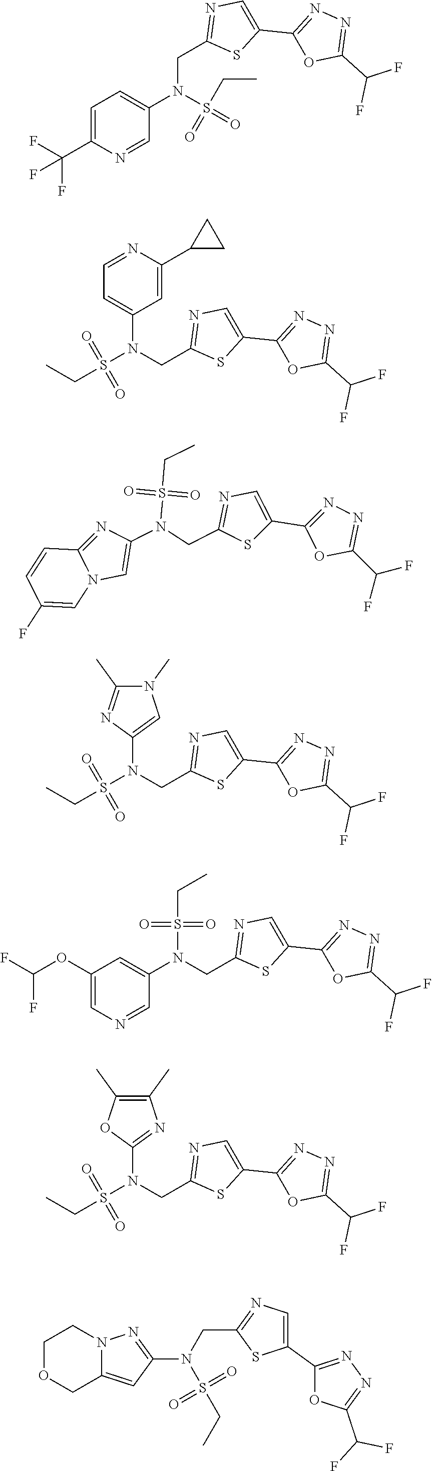

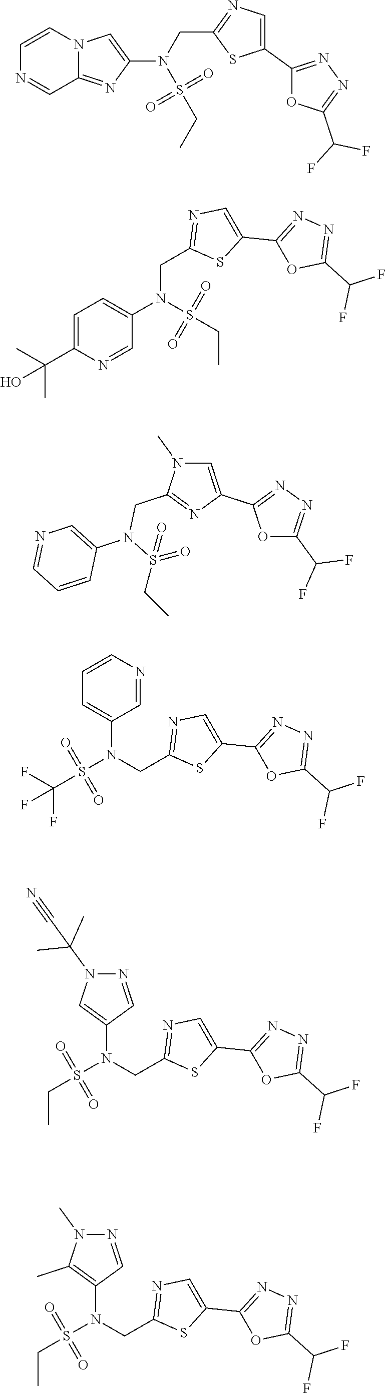 [SMILES: CCS(=O)(=O)N(Cc1ncc(-c2nnc(C(F)F)o2)s1)c1cn2ccncc2n1 ; CCS(=O)(=O)N(Cc1ncc(-c2nnc(C(F)F)o2)s1)c1ccc(C(C)(C)O)nc1 ; CCS(=O)(=O)N(Cc1nc(-c2nnc(C(F)F)o2)cn1C)c1cccnc1 ; O=S(=O)(N(Cc1ncc(-c2nnc(C(F)F)o2)s1)c1cccnc1)C(F)(F)F ; CCS(=O)(=O)N(Cc1ncc(-c2nnc(C(F)F)o2)s1)c1cnn(C(C)(C)C#N)c1 ; CCS(=O)(=O)N(Cc1ncc(-c2nnc(C(F)F)o2)s1)c1cnn(C)c1C]

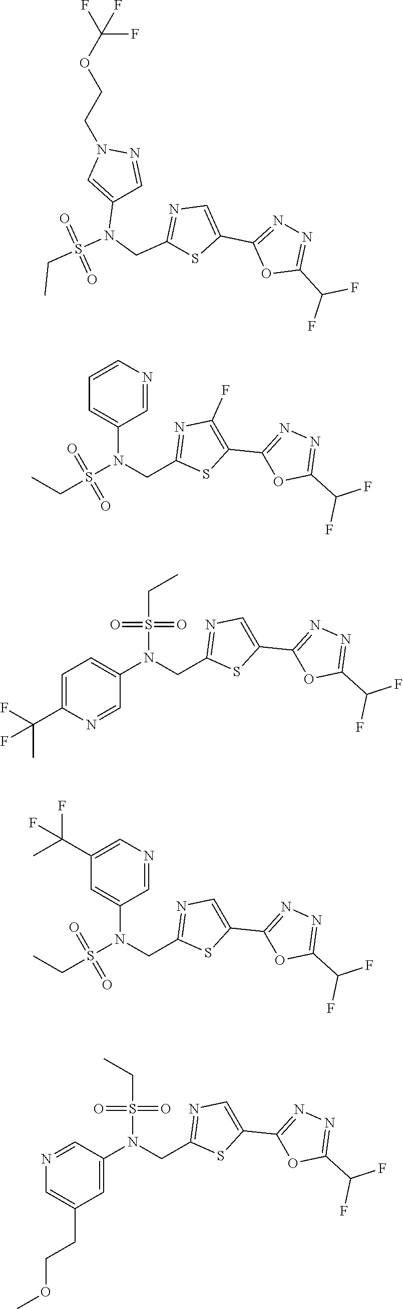 [SMILES: CCS(=O)(=O)N(Cc1ncc(-c2nnc(C(F)F)o2)s1)c1cnn(CCOC(F)(F)F)c1 ; CCS(=O)(=O)N(Cc1nc(F)c(-c2nnc(C(F)F)o2)s1)c1cccnc1 ; CCS(=O)(=O)N(Cc1ncc(-c2nnc(C(F)F)o2)s1)c1ccc(C(C)(F)F)nc1 ; CCS(=O)(=O)N(Cc1ncc(-c2nnc(C(F)F)o2)s1)c1cncc(C(C)(F)F)c1 ; CCS(=O)(=O)N(Cc1ncc(-c2nnc(C(F)F)o2)s1)c1cncc(CCOC)c1]

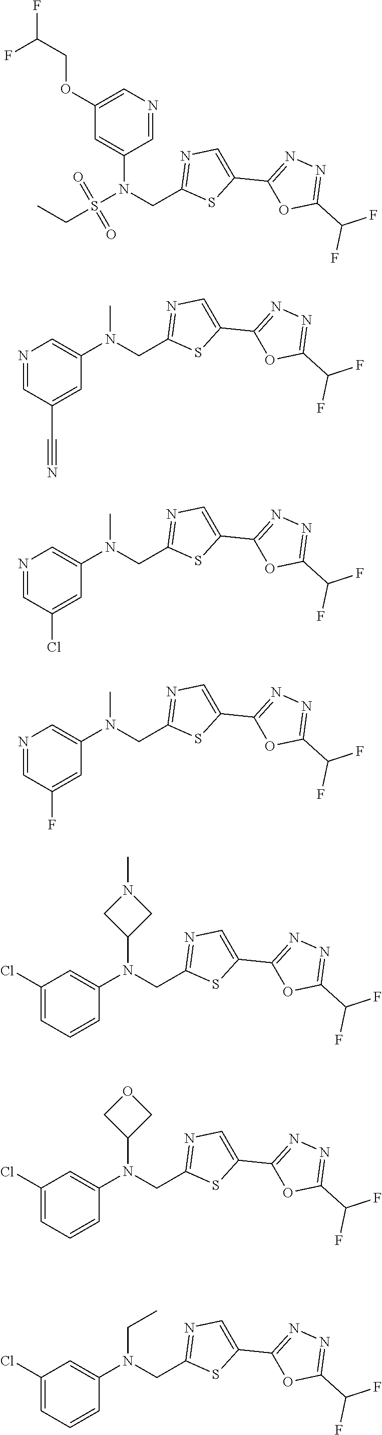

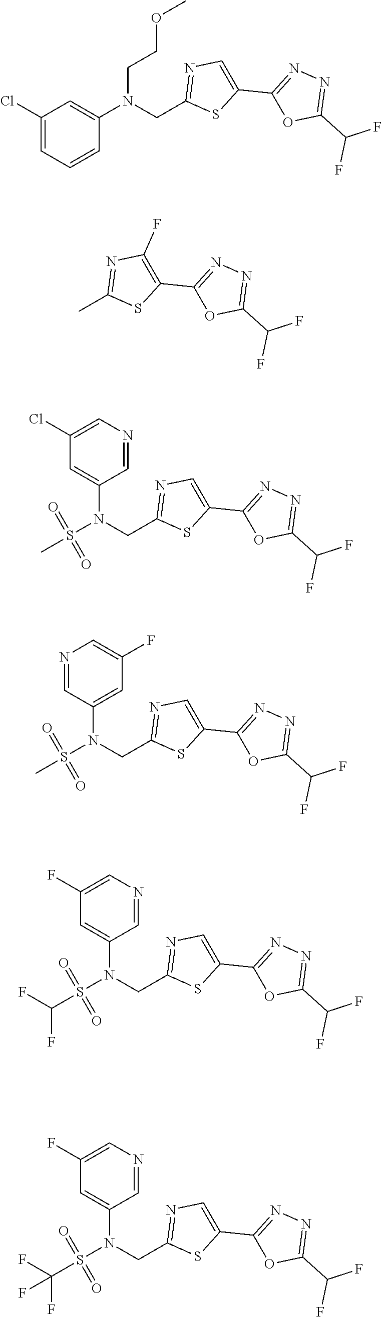 [SMILES: COCCN(Cc1ncc(-c2nnc(C(F)F)o2)s1)c1cccc(Cl)c1 ; Cc1nc(F)c(-c2nnc(C(F)F)o2)s1 ; CS(=O)(=O)N(Cc1ncc(-c2nnc(C(F)F)o2)s1)c1cncc(Cl)c1 ; CS(=O)(=O)N(Cc1ncc(-c2nnc(C(F)F)o2)s1)c1cncc(F)c1 ; O=S(=O)(C(F)F)N(Cc1ncc(-c2nnc(C(F)F)o2)s1)c1cncc(F)c1 ; O=S(=O)(N(Cc1ncc(-c2nnc(C(F)F)o2)s1)c1cncc(F)c1)C(F)(F)F]

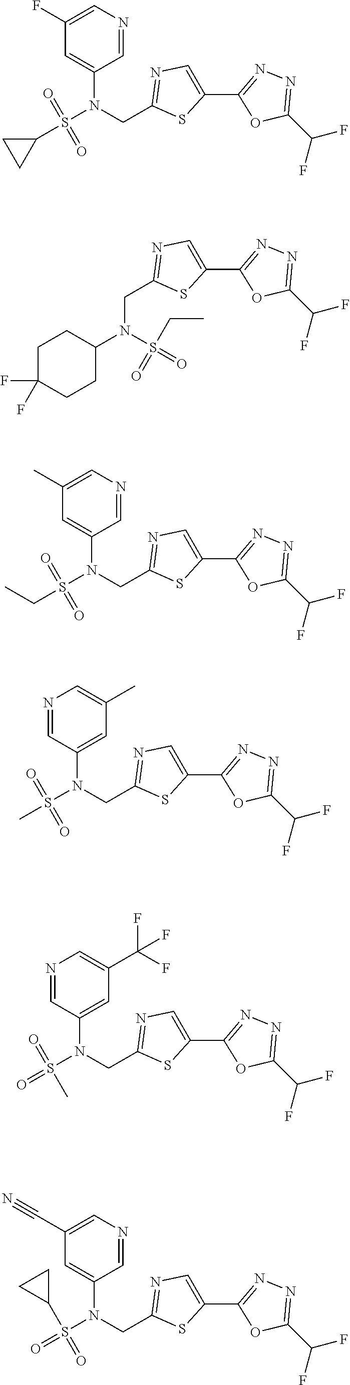 [SMILES: O=S(=O)(C1CC1)N(Cc1ncc(-c2nnc(C(F)F)o2)s1)c1cncc(F)c1 ; CCS(=O)(=O)N(Cc1ncc(-c2nnc(C(F)F)o2)s1)C1CCC(F)(F)CC1 ; CCS(=O)(=O)N(Cc1ncc(-c2nnc(C(F)F)o2)s1)c1cncc(C)c1 ; Cc1cncc(N(Cc2ncc(-c3nnc(C(F)F)o3)s2)S(C)(=O)=O)c1 ; CS(=O)(=O)N(Cc1ncc(-c2nnc(C(F)F)o2)s1)c1cncc(C(F)(F)F)c1 ; N#Cc1cncc(N(Cc2ncc(-c3nnc(C(F)F)o3)s2)S(=O)(=O)C2CC2)c1]

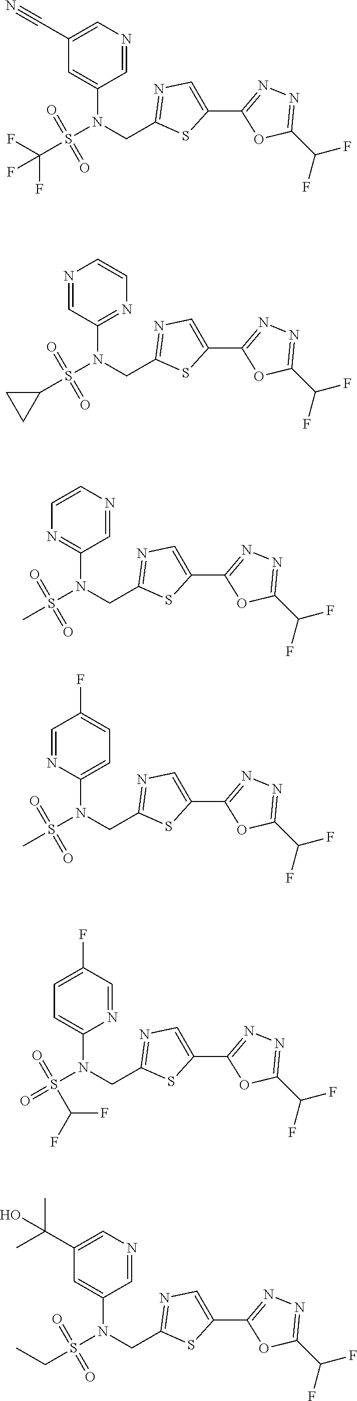 [SMILES: N#Cc1cncc(N(Cc2ncc(-c3nnc(C(F)F)o3)s2)S(=O)(=O)C(F)(F)F)c1 ; O=S(=O)(C1CC1)N(Cc1ncc(-c2nnc(C(F)F)o2)s1)c1cnccn1 ; CS(=O)(=O)N(Cc1ncc(-c2nnc(C(F)F)o2)s1)c1cnccn1 ; CS(=O)(=O)N(Cc1ncc(-c2nnc(C(F)F)o2)s1)c1ccc(F)cn1 ; O=S(=O)(C(F)F)N(Cc1ncc(-c2nnc(C(F)F)o2)s1)c1ccc(F)cn1 ; CCS(=O)(=O)N(Cc1ncc(-c2nnc(C(F)F)o2)s1)c1cncc(C(C)(C)O)c1]

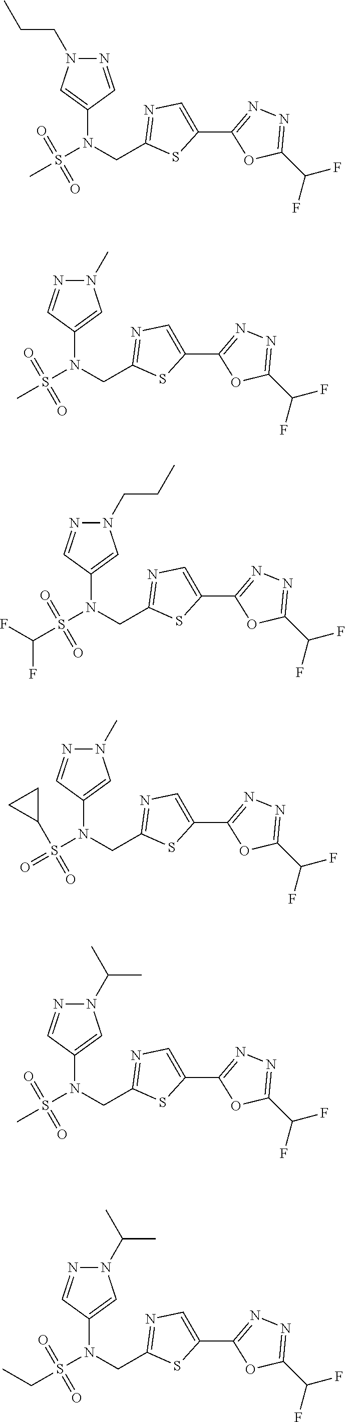 [SMILES: CCCn1cc(N(Cc2ncc(-c3nnc(C(F)F)o3)s2)S(C)(=O)=O)cn1 ; Cn1cc(N(Cc2ncc(-c3nnc(C(F)F)o3)s2)S(C)(=O)=O)cn1 ; CCCn1cc(N(Cc2ncc(-c3nnc(C(F)F)o3)s2)S(=O)(=O)C(F)F)cn1 ; Cn1cc(N(Cc2ncc(-c3nnc(C(F)F)o3)s2)S(=O)(=O)C2CC2)cn1 ; CC(C)n1cc(N(Cc2ncc(-c3nnc(C(F)F)o3)s2)S(C)(=O)=O)cn1 ; CCS(=O)(=O)N(Cc1ncc(-c2nnc(C(F)F)o2)s1)c1cnn(C(C)C)c1]

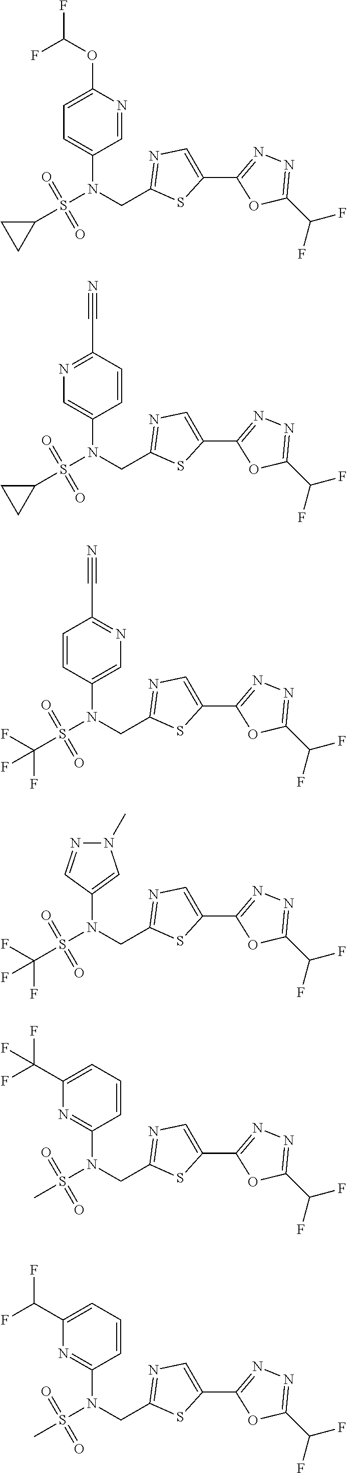

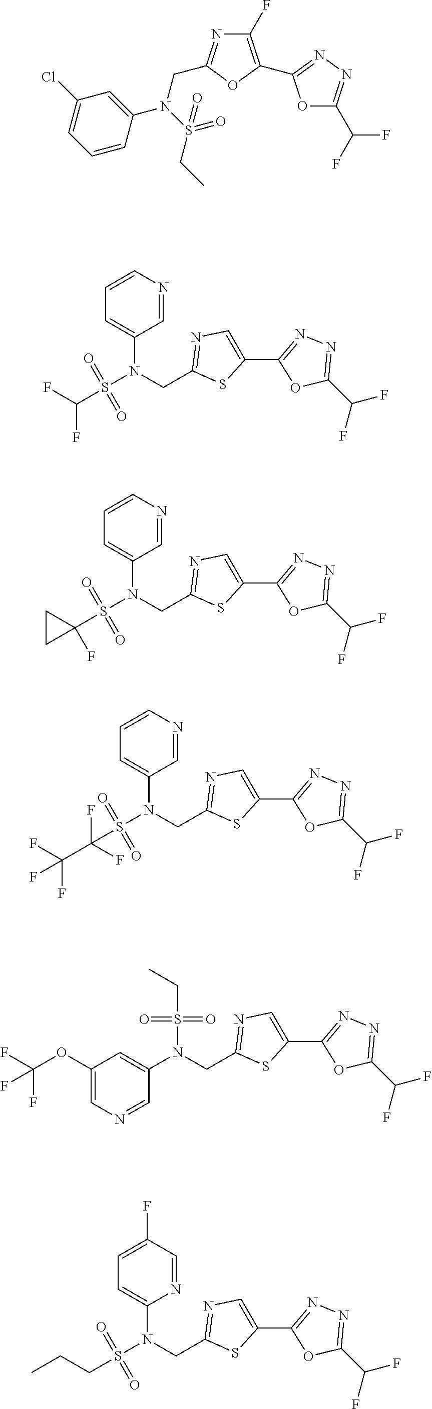 [SMILES: CCS(=O)(=O)N(Cc1nc(F)c(-c2nnc(C(F)F)o2)o1)c1cccc(Cl)c1 ; O=S(=O)(C(F)F)N(Cc1ncc(-c2nnc(C(F)F)o2)s1)c1cccnc1 ; O=S(=O)(N(Cc1ncc(-c2nnc(C(F)F)o2)s1)c1cccnc1)C1(F)CC1 ; O=S(=O)(N(Cc1ncc(-c2nnc(C(F)F)o2)s1)c1cccnc1)C(F)(F)C(F)(F)F ; CCS(=O)(=O)N(Cc1ncc(-c2nnc(C(F)F)o2)s1)c1cncc(OC(F)(F)F)c1 ; CCCS(=O)(=O)N(Cc1ncc(-c2nnc(C(F)F)o2)s1)c1ccc(F)cn1]

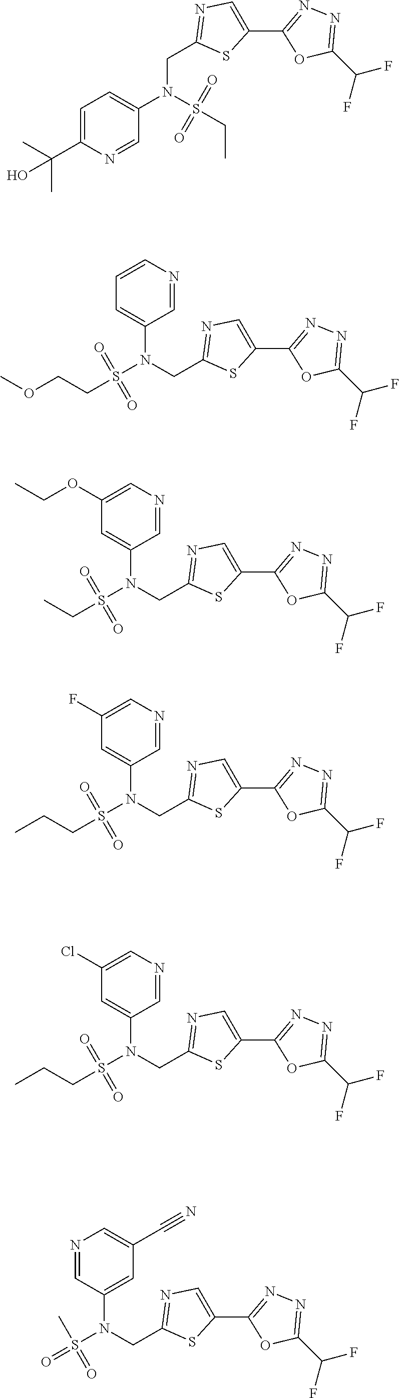 [SMILES: CCS(=O)(=O)N(Cc1ncc(-c2nnc(C(F)F)o2)s1)c1ccc(C(C)(C)O)nc1 ; COCCS(=O)(=O)N(Cc1ncc(-c2nnc(C(F)F)o2)s1)c1cccnc1 ; CCOc1cncc(N(Cc2ncc(-c3nnc(C(F)F)o3)s2)S(=O)(=O)CC)c1 ; CCCS(=O)(=O)N(Cc1ncc(-c2nnc(C(F)F)o2)s1)c1cncc(F)c1 ; CCCS(=O)(=O)N(Cc1ncc(-c2nnc(C(F)F)o2)s1)c1cncc(Cl)c1 ; CS(=O)(=O)N(Cc1ncc(-c2nnc(C(F)F)o2)s1)c1cncc(C#N)c1]

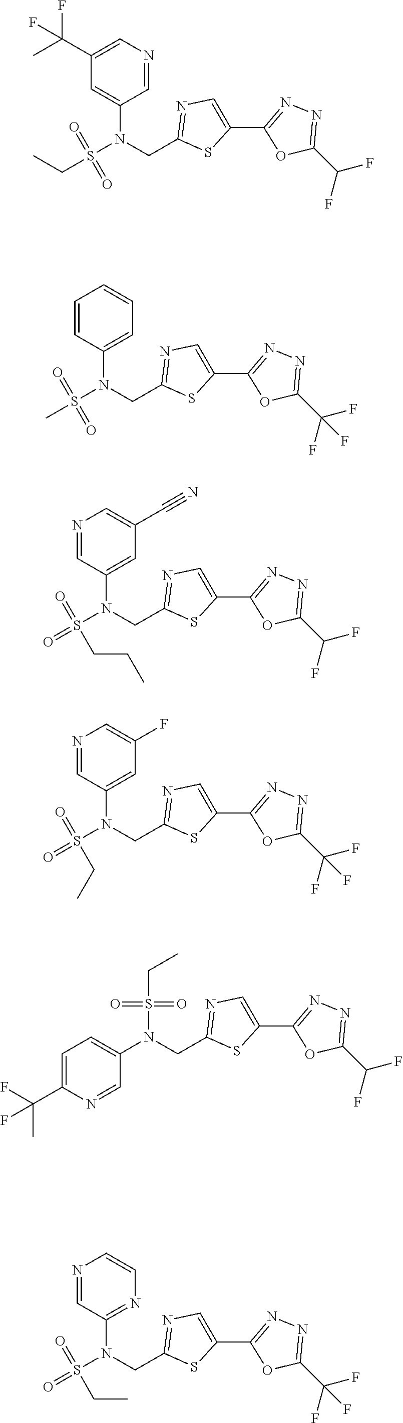

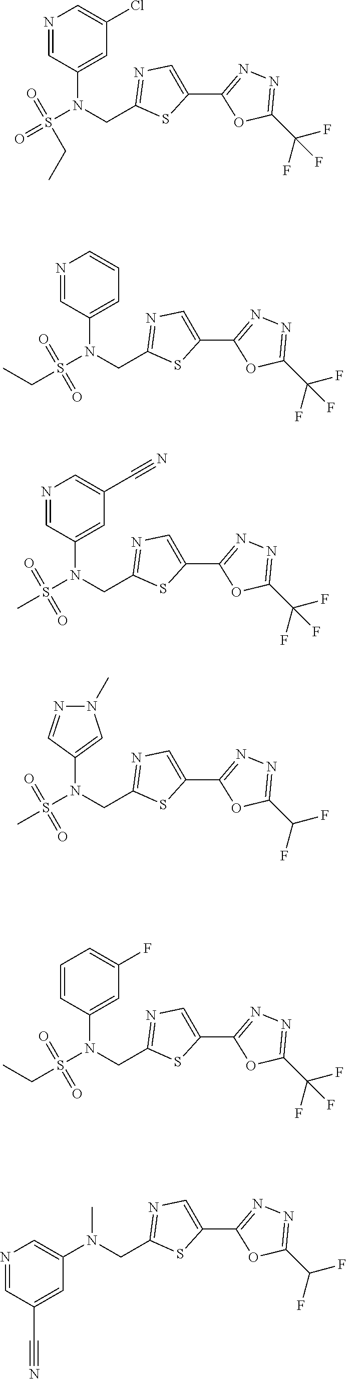 [SMILES: CCS(=O)(=O)N(Cc1ncc(-c2nnc(C(F)(F)F)o2)s1)c1cncc(Cl)c1 ; CCS(=O)(=O)N(Cc1ncc(-c2nnc(C(F)(F)F)o2)s1)c1cccnc1 ; CS(=O)(=O)N(Cc1ncc(-c2nnc(C(F)(F)F)o2)s1)c1cncc(C#N)c1 ; Cn1cc(N(Cc2ncc(-c3nnc(C(F)F)o3)s2)S(C)(=O)=O)cn1 ; CCS(=O)(=O)N(Cc1ncc(-c2nnc(C(F)(F)F)o2)s1)c1cccc(F)c1 ; CN(Cc1ncc(-c2nnc(C(F)F)o2)s1)c1cncc(C#N)c1]

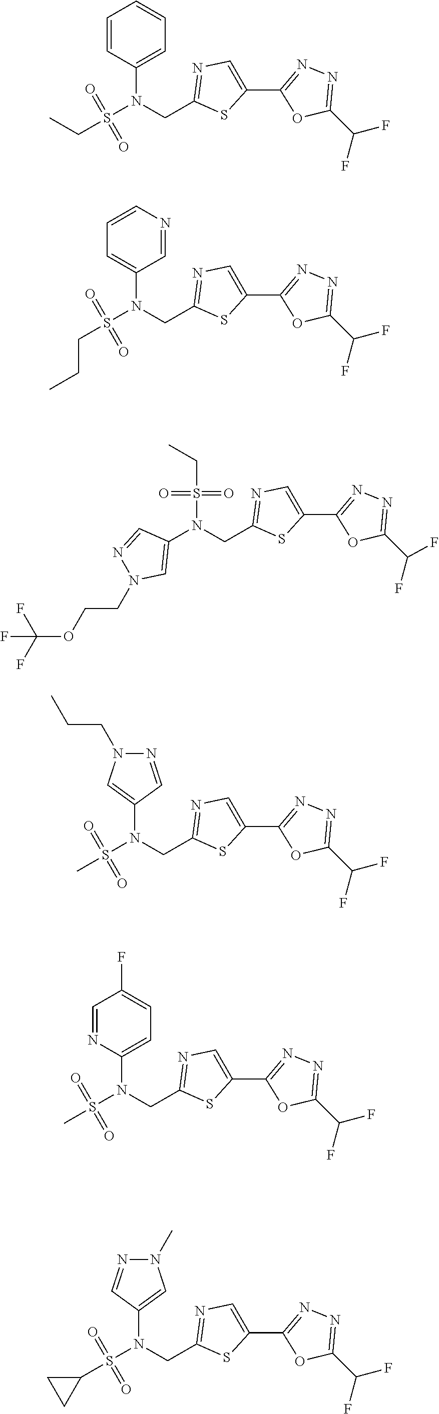

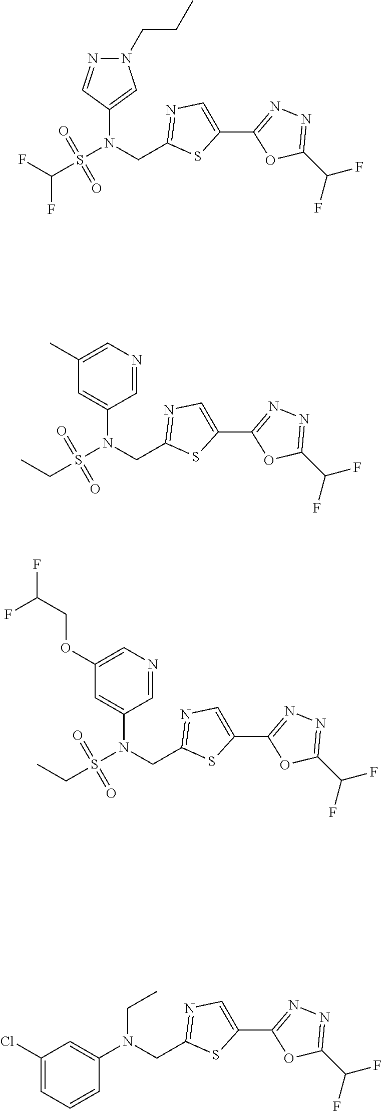 [SMILES: CCCn1cc(N(Cc2ncc(-c3nnc(C(F)F)o3)s2)S(=O)(=O)C(F)F)cn1 ; CCS(=O)(=O)N(Cc1ncc(-c2nnc(C(F)F)o2)s1)c1cncc(C)c1 ; CCS(=O)(=O)N(Cc1ncc(-c2nnc(C(F)F)o2)s1)c1cncc(OCC(F)F)c1 ; CCN(Cc1ncc(-c2nnc(C(F)F)o2)s1)c1cccc(Cl)c1]

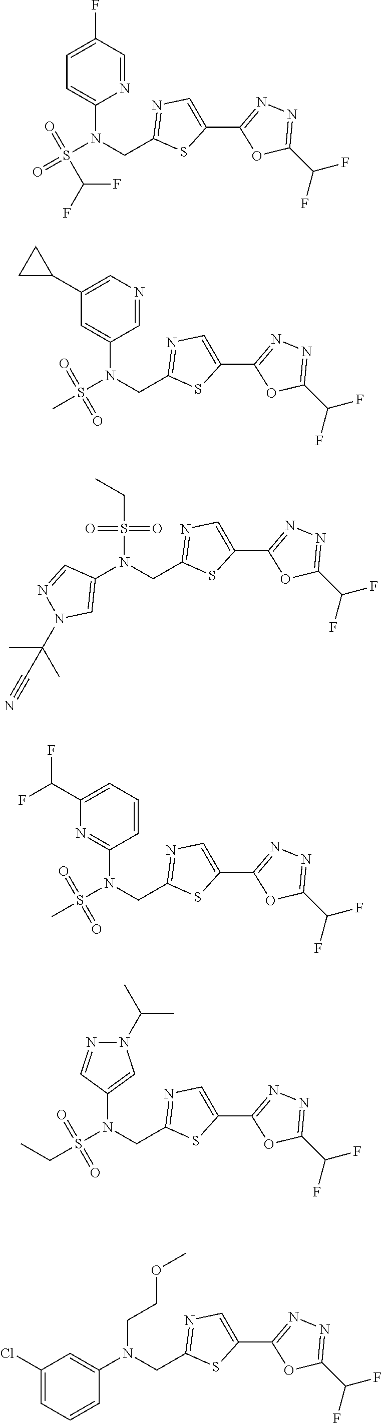

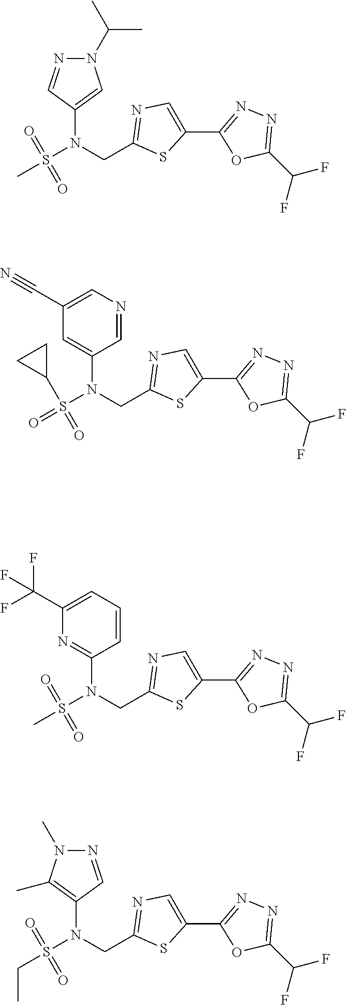 [SMILES: CC(C)n1cc(N(Cc2ncc(-c3nnc(C(F)F)o3)s2)S(C)(=O)=O)cn1 ; N#Cc1cncc(N(Cc2ncc(-c3nnc(C(F)F)o3)s2)S(=O)(=O)C2CC2)c1 ; CS(=O)(=O)N(Cc1ncc(-c2nnc(C(F)F)o2)s1)c1cccc(C(F)(F)F)n1 ; CCS(=O)(=O)N(Cc1ncc(-c2nnc(C(F)F)o2)s1)c1cnn(C)c1C]

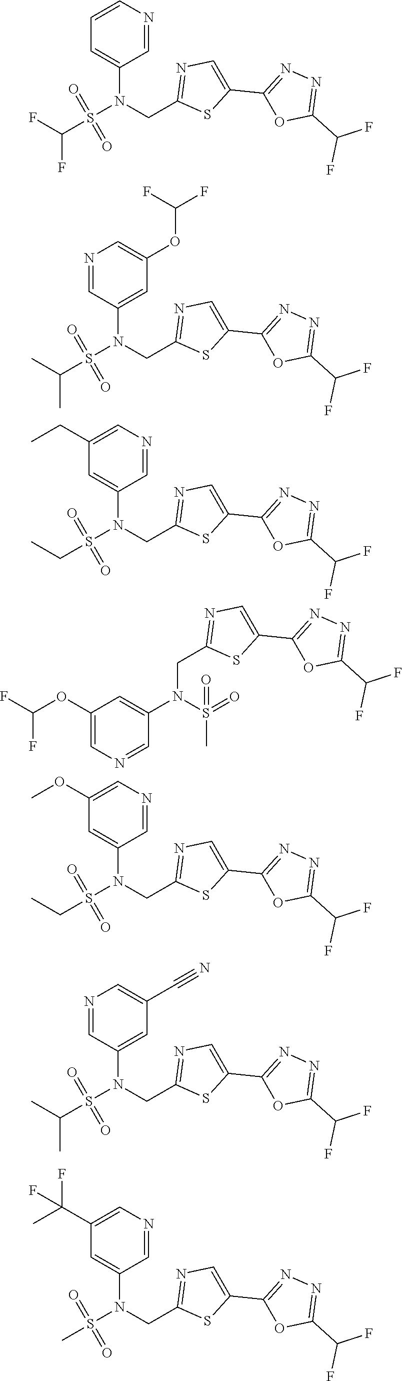

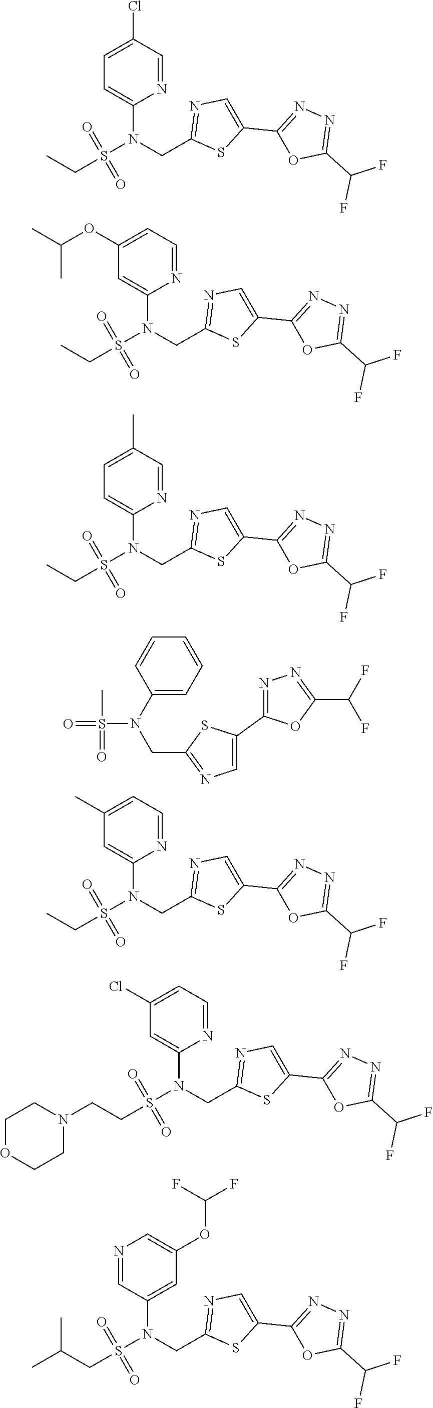

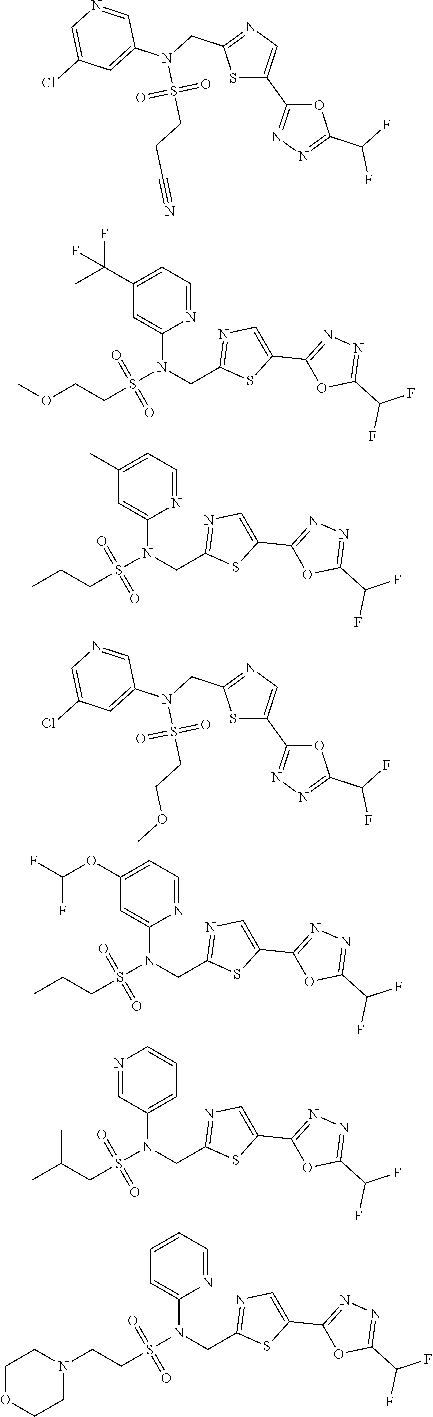

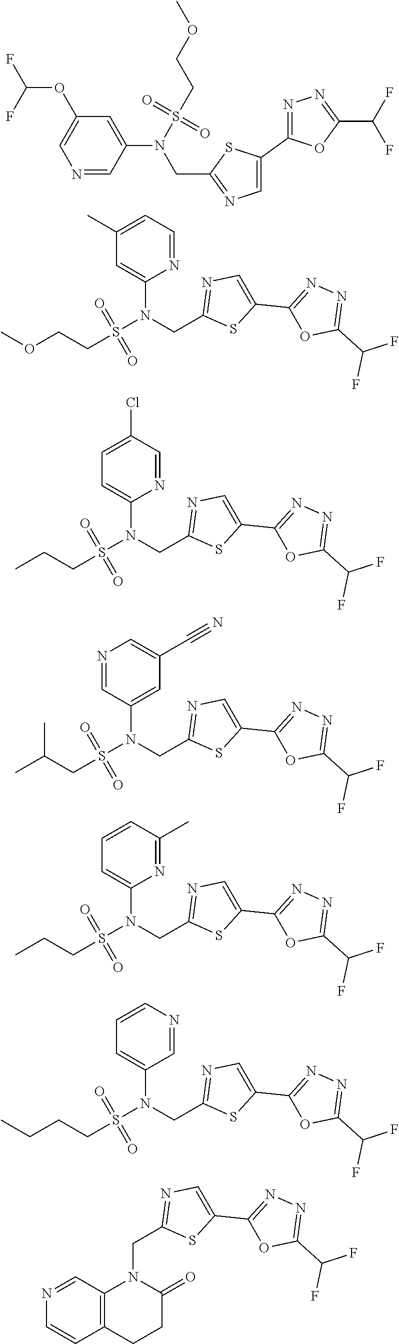

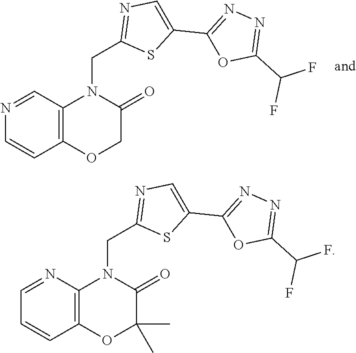 [SMILES: O=C1COc2ccncc2N1Cc1ncc(-c2nnc(C(F)F)o2)s1 ; CC1(C)Oc2cccnc2N(Cc2ncc(-c3nnc(C(F)F)o3)s2)C1=O]

In some embodiments, the present disclosure provides a compound of Formula (Ic) or a salt thereof:

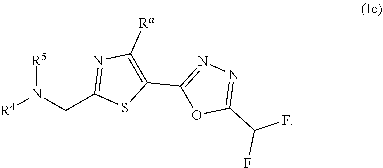

wherein:
R^{a} is H, Me, or F; and
R⁴ and R⁵ are as defined herein, e.g., in Formula (I).

In some embodiments of Formula (Ic), R^{a} is H. In some embodiments, R^{a} is F. In some embodiments, R^{a} is Me.

In some embodiments of Formula (Ic), R⁴ is selected from the group consisting of alkylenealkoxy, alkyleneheterocyclyl, —S(O)₂alkyl, —S(O)₂cycloalkyl, —S(O)₂alkylenecycloalkyl, —S(O)₂alkyleneheterocyclyl, —S(O)₂N(H)alkyleneheterocyclyl, —C(O)alkyl, —C(O)cycloalkyl, —C(O)alkylenecycloalkyl, —C(O)alkyleneheterocyclyl, and —C(O)N(H)alkyleneheterocyclyl. In some embodiments, R⁴ is selected from the group consisting of alkyleneheterocyclyl, —S(O)₂alkyl, —S(O)₂cycloalkyl, —S(O)₂alkyleneheterocyclyl, —C(O)alkyleneheterocyclyl, and —C(O)N(H)alkyleneheterocyclyl. In some embodiments, R⁴ is selected from the group consisting of —S(O)₂alkyl, —S(O)₂cycloalkyl, and —S(O)₂alkyleneheterocyclyl. In some embodiments, R⁴ is —S(O)₂alkyl. In some embodiments, R⁴ is —S(O)₂cycloalkyl. In some embodiments, R⁴ is —S(O)₂N(H)alkyleneheterocyclyl. In some embodiments, the alkylene is a C₁₋₅ alkylene and the heterocyclyl is an optionally substituted 4- to 10-membered heterocyclyl having 1, 2, or 3 heteroatoms selected from the group consisting of N, O, and S. In some embodiments, the alkylene is a C₁₋₅ alkylene and the heterocyclyl is an optionally substituted 4- to 7-membered heterocyclyl having 1, 2, or 3 heteroatoms selected from the group consisting of N, O, and S. In some embodiments, the alkylene is a C₂₋₄ alkylene and the heterocyclyl is an optionally substituted 6-membered heterocyclyl having 1, 2, or 3 heteroatoms selected from the group consisting of N, O, and S.

In some embodiments, the heterocyclyl is selected from the group consisting of piperidine, morpholine, thiomorpholine, thiomorpholine 1-oxide, thiomorpholine 1,1-dioxide, and piperizine, each of which is optionally substituted. In some embodiments, the optional substituent is selected from the group consisting of alkyl, haloalkyl, alkoxy, acyl, sulfonyl, heteroaryl, and heterocyclyl.

In some embodiments of Formula (Ic), R⁵ is selected from the group consisting of:

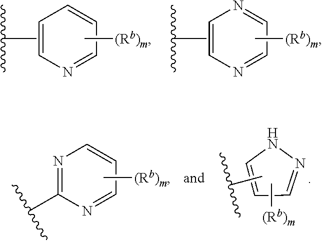

In some embodiments, R⁵ is

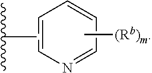

In some embodiments, R⁵ is

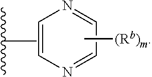

In some embodiments, R⁵ is

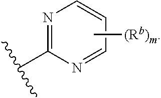

In some embodiments, R⁵ is

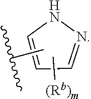

In some embodiments, R^{b} is selected from the group consisting of halogen, haloalkyl, alkyl, Oalkyl, Ohaloalkyl, alkylene-Ohaloalkyl, cycloalkyl, heterocyclyl aryl, heteroaryl, alkylnitrile, or CN. In some embodiments, R^{b} is selected from the group consisting of halogen, alkyl, haloalkyl, alkoxy, haloalkoxy, acyl, sulfonyl, cycloalkyl, heteroaryl, and heterocyclyl. In some embodiments, the haloalkyl is selected from CF₃, CF₂CH₃, CHF₂, or CH₂F. In some embodiments, the alkyl is a —C₁₋₅alkyl. In some embodiments, —C₁₋₅alkyl is methyl, ethyl, propyl, i-propyl, butyl, or t-butyl. In some embodiments, methyl, ethyl, propyl, i-propyl, butyl, or t-butyl is optionally substituted with OH. In some embodiments, the cycloalkyl is a C₃₋₆cycloalkyl. In some embodiments, the aryl is a phenyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the Ohaloalkyl is selected from OCF₃, OCHF₂, or OCH₂F. In some embodiments, the Oalkyl is O-methyl, O-ethyl, O-propyl, O-i-propyl, O-butyl, or O-t-butyl. In some embodiments, R^{b} is selected from the group consisting of F, Cl, —CH₃, —CH₂CH₃, —CF₃, —CHF₂, —CF₂CH₃, —CN, —OCH₃, —OCH₂CH₃, —OCH(CH₃)₂, —OCHF₂, —OCH₂CF₂H, and cyclopropyl. In some embodiments, m is 0, 1, or 2. In some embodiments, m is 0 or 1. In some embodiments, m is 0. In some embodiments, m is 1. In some embodiments, m is 2.

In some embodiments, the present disclosure provides a compound of Formula (Id) or a salt thereof:

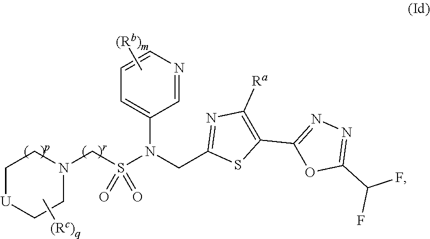

wherein:
U is NR^{d}, O, S, S(O), S(O)₂, CH₂, CHF, or CF₂;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), —S(O₂)R^{e}, cycloalkyl, heteroaryl, or heterocyclyl;
R^{c} is each independently F, alkyl, haloalkyl, alkoxy, haloalkoxy, —C(O)R^{e}, —C(O)OR^{e}, —C(O)N(R^{e})(R^{e′}), —S(O₂)R^{e}, heteroaryl, or heterocyclyl, and/or two R^{c} groups taken together with the carbon atoms to which they are attached form a bridged or fused C₃₋₇ cycloalkyl, a bridged or fused 4- to 7-membered heterocyclyl; or a 5- or 6-membered heteroaryl, each of which is optionally substituted;
R^{d} is H, alkyl, acyl, sulfonyl, cycloalkyl, aryl, or heteroaryl;
R^{e} and R^{e′} is each independently H, alkyl, cycloalkyl, heterocyclyl, aryl, heteroaryl, —CH₂cycloalkyl, —CH₂heterocyclyl, —CH₂aryl, or —CH₂heteroaryl;
m is 0, 1, 2, or 3;
p is 0, 1, 2, or 3;
q is 0, 1, or 2; and
r is 1, 2, 3, or 4.

In some embodiments, the present disclosure provides a compound of Formula (Ie) or a salt thereof:

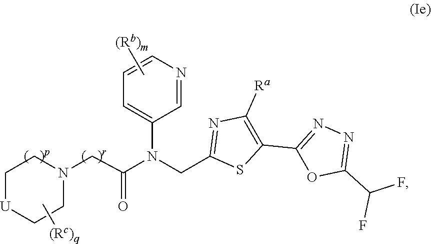

wherein:
U is NR^{d}, O, S, S(O), S(O)₂, CH₂, CHF, or CF₂;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), sulfonyl, cycloalkyl, heteroaryl, or heterocyclyl;
R^{c} is each independently F, alkyl, haloalkyl, alkoxy, haloalkoxy, —C(O)R^{e}, —C(O)OR^{e}, —C(O)N(R^{e})(R^{e′}), —S(O₂)R^{e}, heteroaryl, or heterocyclyl, and/or two R^{c} groups taken together with the carbon atoms to which they are attached form a bridged or fused C₃₋₇ cycloalkyl, a bridged or fused 4- to 6-membered heterocyclyl; or a 5- or 6-membered heteroaryl, each of which is optionally substituted;
R^{d} is H, alkyl, acyl, sulfonyl, cycloalkyl, aryl, or heteroaryl;
R^{e} and R^{e′} is each independently H, alkyl, cycloalkyl, heterocyclyl, aryl, heteroaryl, —CH₂cycloalkyl, —CH₂heterocyclyl, —CH₂aryl, or —CH₂heteroaryl;
m is 0, 1, 2, or 3;
p is 0, 1, 2, or 3;
q is 0, 1, or 2; and
r is 1, 2, 3, or 4.

In some embodiments, the present disclosure provides a compound of Formula (If) or a salt thereof:

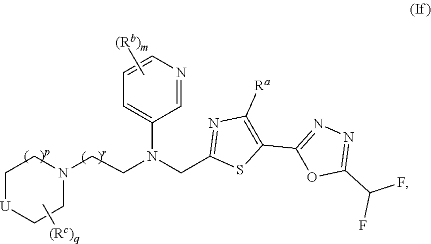

wherein:
U is NR^{d}, O, S, S(O), S(O)₂, CH₂, CHF, or CF₂;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), sulfonyl, cycloalkyl, heteroaryl, or heterocyclyl;
R^{c} is each independently F, alkyl, haloalkyl, alkoxy, haloalkoxy, —C(O)R^{e}, —C(O)OR^{e}, —C(O)N(R^{e})(R^{e′}), —S(O₂)R^{e}, heteroaryl, or heterocyclyl, and/or two R^{c} groups taken together with the carbon atoms to which they are attached form a bridged or fused C₃₋₇ cycloalkyl, a bridged or fused 4- to 7-membered heterocyclyl; or a 5- or 6-membered heteroaryl, each of which is optionally substituted;
R^{d} is H, alkyl, acyl, sulfonyl, cycloalkyl, aryl, or heteroaryl;
R^{e} and R^{e′} is each independently H, alkyl, cycloalkyl, heterocyclyl, aryl, heteroaryl, —CH₂cycloalkyl, —CH₂heterocyclyl, —CH₂aryl, or —CH₂heteroaryl;
m is 0, 1, 2, or 3;
p is 0, 1, 2, or 3;
q is 0, 1, or 2; and
r is 1, 2, 3, or 4.

In some embodiments, the present disclosure provides a compound of Formula (Ig) or a salt thereof:

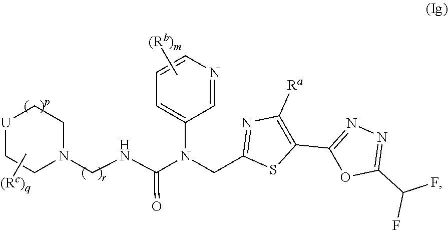

wherein:
U is NR^{d}, O, S, S(O), S(O)₂, CH₂, CHF, or CF₂;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), sulfonyl, cycloalkyl, heteroaryl, or heterocyclyl;
R^{c} is each independently F, alkyl, haloalkyl, alkoxy, haloalkoxy, —C(O)R^{e}, —C(O)OR^{e}, —C(O)N(R^{e})(R^{e′}), —S(O₂)R^{e}, heteroaryl, or heterocyclyl, and/or two R^{c} groups taken together with the carbon atoms to which they are attached form a bridged or fused C₃₋₇ cycloalkyl, a bridged or fused 4- to 7-membered heterocyclyl; or a 5- or 6-membered heteroaryl, each of which is optionally substituted;
R^{d} is H, alkyl, acyl, sulfonyl, cycloalkyl, aryl, or heteroaryl;
R^{e} and R^{e′} is each independently H, alkyl, cycloalkyl, heterocyclyl, aryl, heteroaryl, —CH₂cycloalkyl, —CH₂heterocyclyl, —CH₂aryl, or —CH₂heteroaryl;
m is 0, 1, 2, or 3;
p is 0, 1, 2, or 3;
q is 0, 1, or 2; and
r is 1, 2, 3, or 4.

In some embodiments, the compound has the formula:

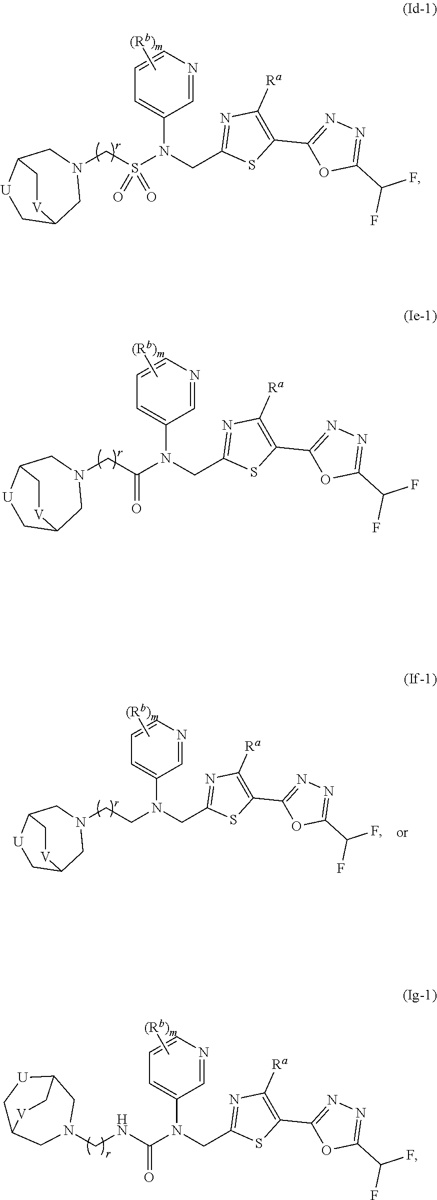

wherein:
U, R^{a}, R^{b}, m, and r are as defined above in Formulas (Id), (Ie), (If), and (Ig); and V is O or NR^{d}.

In some embodiments of Formulas (Id)-(Ig) and (Id-1)-(Ig-1), U is NR^{d}, O, or S and V is O. In some embodiments, U is N, O, or S and V is NR^{d}. In some embodiments, U is NR^{d} and V is NR^{d}. In some embodiments, U is O and V is NR^{d}. In some embodiments, U is S and V is NR^{d}. In some embodiments, U is NR^{d} and V is O. In some embodiments, U is O and V is O. In some embodiments, U is S and V is O.

In some embodiments of Formulas (Id)-(Ig) and (Id-1)-(Ig-1), U is O, S, S(O)₂, CH₂, or NR^{d}. In some embodiments, U is O, S, CH₂, or NR^{d}. In some embodiments, U is O, S, or NR^{d}. In some embodiments, U is O or CH₂. In some embodiments, U is O. In some embodiments, U is S. In some embodiments, U is NR^{d}. In some embodiments, U is S(O)₂.

In some embodiments of Formulas (Id)-(Ig) and (Id-1)-(Ig-1), R^{a} is H. In some embodiments, R^{a} is F. In some embodiments, R^{a} is Me.

In some embodiments of Formulas (Id)-(Ig) and (Id-1)-(Ig-1), R^{b} is halo, alkyl, haloalkyl, alkyl, haloalkoxy, cycloalkyl, heterocyclyl, heteroaryl, or nitrile. In some embodiments, R^{b} is halo, alkyl, haloalkyl, alkyl, haloalkoxy, cycloalkyl, or nitrile. In some embodiments, the haloalkyl is selected from CF₃, CF₂CH₃, CHF₂, or CH₂F. In some embodiments, the alkyl is a —C₁₋₅alkyl. In some embodiments, —C₁₋₅alkyl is methyl, ethyl, propyl, i-propyl, butyl, or t-butyl. In some embodiments, the cycloalkyl is a C₃₋₆cycloalkyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the haloalkoxy is selected from OCF₃, OCHF₂, or OCH₂F. In some embodiments, the alkoxy is O-methyl, O-ethyl, O-propyl, O-i-propyl, O-butyl, or O-t-butyl. In some embodiments, R^{b} is —C(O)R^{e}, —C(O)OR, —C(O)N(R^{e})(R^{e′}).

In some embodiments of Formulas (Id)-(Ig), R^{c} is F, C₁₋₅ alkyl, haloalkyl, C₁₋₅ alkoxy, haloalkoxy, acyl, sulfonyl, 5- or 6-membered heteroaryl, or C₃₋₆ heterocyclyl. In some embodiments, R^{c} is —C(O)R^{e}, —C(O)OR^{e}, —C(O)N(R^{e})(R^{e′}). In some embodiments, two R^{e} groups taken together with the carbon atoms to which they are attached form a bridged or fused C₃₋₇ cycloalkyl, a bridged or fused 5- or 6-membered heterocyclyl, or a 5- or 6-membered heteroaryl, each of which is optionally substituted. In some embodiments, two R^{c} groups taken together with the carbon atoms to which they are attached form an optionally substituted bridged or fused C₃₋₇ cycloalkyl. In some embodiments, two R^{c} groups taken together with the carbon atoms to which they are attached form an optionally substituted bridged or fused 5- or 6-membered heterocyclyl. In some embodiments, two R^{c} groups taken together with the carbon atoms to which they are attached form an alkoxy or aminoalkyl bridge. In some embodiments, the optional substituent is one or more R^{b}, as defined above. In some embodiments, the optional substituent is selected from the group consisting of F, C₁₋₅ alkyl, C₁₋₅ alkoxy, CF₃, CF₂H, CFH₂, —OCF₃, —OCF₂H, —OCFH₂, —C(O)R^{e}, —C(O)OR^{e}, —C(O)N(R^{e})(R^{e′}), and —SO₂R^{e}. In some embodiments, the optional substituent is selected from the group consisting of F, C₁₋₅ alkyl, C₁₋₅ alkoxy, CF₃, CF₂H, CFH₂, —OCF₃, —OCF₂H, and —OCFH₂. In some embodiments, the optional substituent is F or C₁₋₅ alkyl. In some embodiments, the optional substituent is F.

In some embodiments, the optional substituent is C₁₋₅ alkyl. In some embodiments, the C₁₋₅ alkyl is methyl. In some embodiments, the C₁₋₅ alkyl is ethyl. In some embodiments, the C₁₋₅ alkyl is propyl. In some embodiments, the C₁₋₅ alkyl is isopropyl.

In some embodiments of Formulas (Id)-(Ig) and (Id-1)-(Ig-1), R^{e} and R^{e′} is each independently H, alkyl, cycloalkyl, or —CH₂cycloalkyl. In some embodiments, the alkyl is a —C₁₋₅alkyl. In some embodiments, —C₁₋₅alkyl is methyl, ethyl, propyl, i-propyl, butyl, or t-butyl. In some embodiments, the cycloalkyl is a C₃₋₆cycloalkyl. In some embodiments, the cycloalkyl is cyclopropyl. In some embodiments, R^{e} and R^{e′} are H.

In some embodiments of Formulas (Id)-(Ig) and (Id-1)-(Ig-1), m is 0, 1, or 2. In some embodiments, m is 0 or 1. In some embodiments, m is 0. In some embodiments, m is 1. In some embodiments, m is 2.

In some embodiments of Formulas (Id)-(Ig), p is 0, 1, or 2. In some embodiments, p is 0 or 1. In some embodiments, p is 1 or 2. In some embodiments, p is 0. In some embodiments, p is 1. In some embodiments, p is 2.

In some embodiments of Formulas (Id)-(Ig) and (Id-1)-(Ig-1), r is 1, 2, or 3. In some embodiments, r is 1 or 2. In some embodiments, r is 2 or 3. In some embodiments, r is 1. In some embodiments, r is 2. In some embodiments, r is 3. In some embodiments, r is 4.

In some embodiments of Formulas (Id)-(Ig), q is 0 or 1. In some embodiments, q is 0. In some embodiments, q is 1. In some embodiments, q is 2.

In some embodiments of Formulas (Id)-(Ig), r is 1 and p is 1. In some embodiments, r is 2 and p is 1. In some embodiments, r is 3 and p is 1.

In some embodiments, the present disclosure provides a compound of Formula (Ih) or a salt thereof:

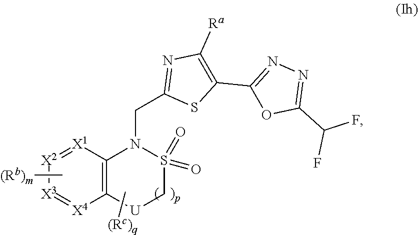

wherein:
U is NR^{d}, O, S, S(O), S(O)₂, CH₂, CHF, or CF₂;
X¹, X², X³, and X⁴ is each independently CH or N;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), —SO₂R^{e}, cycloalkyl, heteroaryl, or heterocyclyl;
R^{c} is each independently F, alkyl, haloalkyl, alkoxy, or haloalkoxy, and/or two R^{c} groups taken together with the atoms to which they are attached form an optionally substituted C₃₋₇ cycloalkyl;
R^{d} is H, alkyl, acyl, sulfonyl, cycloalkyl, aryl, or heteroaryl;
R^{e} and R^{e′} is each independently H, alkyl, cycloalkyl, heterocyclyl, aryl, heteroaryl, —CH₂cycloalkyl, —CH₂heterocyclyl, —CH₂aryl, or —CH₂heteroaryl;
m is 0, 1, 2, or 3;
p is 0, 1, 2, or 3; and
q is 0, 1, or 2.

In some embodiments, the present disclosure provides a compound of Formula (Ii) or a salt thereof:

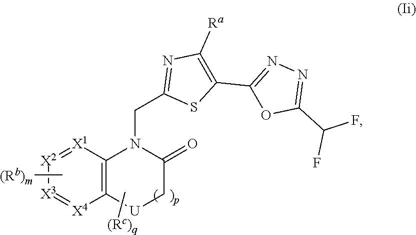

wherein:
U is NR^{d}, O, S, S(O), S(O)₂, CH₂, CHF, or CF₂;
X¹, X², X³, and X⁴ is each independently CH or N;
R^{a} is H, Me, or F;
R^{b} is each independently halo, alkyl, haloalkyl, alkoxy, haloalkoxy, —C(O)R^{e}, —C(O)OR, —C(O)N(R^{e})(R^{e′}), —SO₂R, cycloalkyl, heteroaryl, or heterocyclyl;
R^{c} is each independently F, alkyl, haloalkyl, alkoxy, or haloalkoxy, and/or two R^{c} groups taken together with the atoms to which they are attached form an optionally substituted C₃₋₇ cycloalkyl;
R^{d} is H, alkyl, —C(O)R^{e}, sulfonyl, cycloalkyl, aryl, or heteroaryl;
R^{e} and R^{e′} is each independently H, alkyl, cycloalkyl, heterocyclyl, aryl, heteroaryl, —CH₂cycloalkyl, —CH₂heterocyclyl, —CH₂aryl, or —CH₂heteroaryl;
m is 0, 1, 2, or 3;
p is 0, 1, 2, or 3; and
q is 0, 1, or 2.

In some embodiments, the present disclosure provides a compound of Formula (Ij) or a salt thereof:

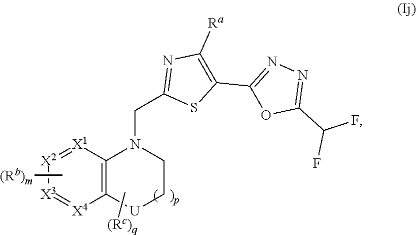

wherein:
U is NR^{d}, O, S, S(O), S(O)₂, CH₂, CHF, or CF₂;
X¹, X², X³, and X⁴ is each independently CH or N;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), —SO₂R, cycloalkyl, heteroaryl, or heterocyclyl;
R^{c} is each independently F, alkyl, haloalkyl, alkoxy, or haloalkoxy, and/or two R^{c} groups taken together with the atoms to which they are attached form an optionally substituted C₃₋₇ cycloalkyl;
R^{d} is H, alkyl, —C(O)R^{e}, sulfonyl, cycloalkyl, aryl, or heteroaryl;
R^{e} and R^{e′} is each independently H, alkyl, cycloalkyl, heterocyclyl, aryl, heteroaryl, —CH₂cycloalkyl, —CH₂heterocyclyl, —CH₂aryl, or —CH₂heteroaryl;
m is 0, 1, 2, or 3;
p is 0, 1, 2, or 3; and
q is 0, 1, or 2.

In some embodiments of Formulas (Ih)-(Ij), NR^{d}, O, S, S(O)₂, or CH₂. In some embodiments, U is NR^{d}, O, S, or CH₂. In some embodiments, U is O or CH₂. In some embodiments, U is O. In some embodiments, U is CH₂. In some embodiments, U is S. In some embodiments, U is S(O)₂. In some embodiments, U is NR^{d}.

In some embodiments of Formulas (Ih)-(Ij), each of X¹, X², X³, and X⁴ is CH. In some embodiments, one of X¹, X², X³, and X⁴ is N. In some embodiments, two of X¹, X², X³, and X₄ are N. In some embodiments, X¹ is N and each of X², X³, and X⁴ is CH. In some embodiments, X² is N and each of X¹, X³, and X⁴ is CH. In some embodiments, X³ is N and each of X¹, X², and X⁴ is CH. In some embodiments, X⁴ is N and each of X¹, X², and X³ is CH.

In some embodiments of Formulas (Ih)-(Ij), U is CH₂ and one of X¹, X², X³, and X⁴ is N. In some embodiments, U is CH₂, X¹ is N and each of X², X³, and X⁴ is CH. In some embodiments, U is CH₂, X² is N and each of X¹, X³, and X⁴ is CH. In some embodiments, U is CH₂, X³ is N and each of X¹, X², and X⁴ is CH. In some embodiments, U is CH₂, X⁴ is N and each of X¹, X², and X³ is CH. In some embodiments, p is 0. In some embodiments, p is 1.

In some embodiments of Formulas (Ih)-(Ij), U is O and one of X¹, X², X³, and X⁴ is N. In some embodiments, U is O, X¹ is N and each of X², X³, and X⁴ is CH. In some embodiments, U is O, X² is N and each of X¹, X³, and X⁴ is CH. In some embodiments, U is O, X³ is N and each of X¹, X², and X⁴ is CH. In some embodiments, U is O, X⁴ is N and each of X¹, X², and X³ is CH.

In some embodiments of Formulas (Ih)-(Ij), R^{a} is H. In some embodiments, R^{a} is F. In some embodiments, R^{a} is Me.

In some embodiments of Formulas (Ih)-(Ij), R^{b} is halo, alkyl, haloalkyl, alkyl, haloalkoxy, cycloalkyl, heterocyclyl, heteroaryl, or nitrile. In some embodiments, R^{b} is halo, alkyl, haloalkyl, alkyl, haloalkoxy, cycloalkyl, or nitrile. In some embodiments, the haloalkyl is selected from CF₃, CF₂CH₃, CHF₂, or CH₂F. In some embodiments, the alkyl is a —C₁₋₅alkyl. In some embodiments, —C₁₋₅alkyl is methyl, ethyl, propyl, i-propyl, butyl, or t-butyl. In some embodiments, the cycloalkyl is a C₃₋₆cycloalkyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the haloalkoxy is selected from OCF₃, OCHF₂, or OCH₂F. In some embodiments, the alkoxy is O-methyl, O-ethyl, O-propyl, O-i-propyl, O-butyl, or O-t-butyl.

In some embodiments of Formulas (Ih)-(Ij), R^{c} is F, C₁₋₅ alkyl, haloalkyl, C₁₋₅ alkoxy, haloalkoxy, acyl, sulfonyl, 5- or 6-membered heteroaryl, or C₃₋₆ heterocyclyl. In some embodiments, R^{c} is F, C₁₋₅ alkyl, haloalkyl, C₁₋₅ alkoxy, or haloalkoxy. In some embodiments, R^{c} is F or C₁₋₅ alkyl. In some embodiments, R^{c} is F or methyl. In some embodiments, R^{c} is F. In some embodiments, R^{c} is methyl. In some embodiments, the two R^{c} groups are attached to the same carbon atom, which can also be referred to as germinal substitution. In some embodiments, two R^{c} groups taken together with the atoms to which they are attached form an optionally substituted C₃₋₆ cycloalkyl. In some embodiments, two R^{c} groups taken together with the atoms to which they are attached form an optionally substituted cyclopropyl. In some embodiments, the optional substituent is one or more R^{b}, as defined above. In some embodiments, the optional substituent is selected from the group consisting of F, C₁₋₅ alkyl, C₁₋₅ alkoxy, CF₃, CF₂H, CFH₂, —OCF₃, —OCF₂H, —OCFH₂, —C(O)R^{e}, —C(O)OR^{e}, —C(O)N(R^{e})(R′), and —SO₂R^{e}. In some embodiments, the optional substituent is selected from the group consisting of F, C₁₋₅ alkyl, C₁₋₅ alkoxy, CF₃, CF₂H, CFH₂, —OCF₃, —OCF₂H, and —OCFH₂. In some embodiments, the optional substituent is F or C₁₋₅ alkyl. In some embodiments, the optional substituent is F. In some embodiments, the optional substituent is C₁₋₅ alkyl. In some embodiments, the C₁₋₅ alkyl is methyl. In some embodiments, the C₁₋₅ alkyl is ethyl. In some embodiments, the C₁₋₅ alkyl is propyl. In some embodiments, the C₁₋₅ alkyl is isopropyl. In some embodiments, two optional substituents are attached to the same carbon, which is also referred to as germinal substitution.

In some embodiments of Formulas (Ih)-(Ij), when U is NR^{d}, an R^{d} and R^{e}taken together with the atoms to which they are attached form a 5- to 7-membered heterocyclyl. In some embodiments, an R^{d} and R^{e}taken together with the atoms to which they are attached form a 6-membered heterocyclyl. In some embodiments, the heterocyclyl comprises 1 or 2 heteroatoms selected from N, O, and S.

In some embodiments, the present disclosure provides a compound of Formula (Ih-1), Formula (Ii-1), or Formula (Ij-1):

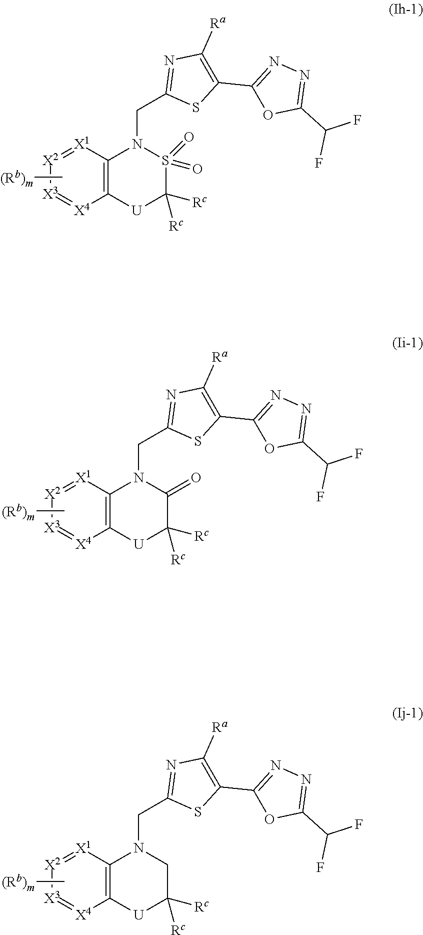

wherein R^{a}, R^{b}, R^{e}, X¹, X², X³, X⁴, U, and m are as defined above in Formula (Ih), Formula (Ii), and Formula (Ij).

In some embodiments of Formula (Ih-1), Formula (Ii-1), and Formula (Ij-1), each R^{e} is F. In some embodiments, each R^{c} is Me. In some embodiments, two R^{c} groups taken together with the carbon atoms to which they are attached form an optionally substituted C₃₋₆ cycloalkyl. In some embodiments, two R^{c} groups taken together with the carbon atoms to which they are attached form a cyclopropyl or cyclobutyl, each of which is optionally substituted. In some embodiments, two R^{c} groups taken together with the carbon atoms to which they are attached form an optionally substituted cyclopropyl. In some embodiments, the optional substituent is F or C₁₋₅ alkyl. In some embodiments, the optional substituent is F. In some embodiments, the optional substituent is C₁₋₅ alkyl. In some embodiments, the C₁₋₅ alkyl is methyl. In some embodiments, the C₁₋₅ alkyl is ethyl. In some embodiments, the C₁₋₅ alkyl is propyl. In some embodiments, the C₁₋₅ alkyl is isopropyl. In some embodiments, two optional substituents are attached to the same carbon, which is also referred to as germinal substitution.

In some embodiments, R^{d} is H, alkyl, or cycloalkyl. In some embodiments, R^{d} is H. In some embodiments, R^{d} is alkyl. In some embodiments, R^{d} is cycloalkyl. In some embodiments, alkyl is methyl, ethyl, propyl, isopropyl, or t-butyl. In some embodiments, the cycloalkyl is cyclopropyl, cyclopentyl, or cyclohexyl.

In some embodiments, m is 0, 1, or 2. In some embodiments, m is 0 or 1. In some embodiments, m is 0. In some embodiments, m is 1. In some embodiments, m is 2.

In some embodiments, p is 0, 1, or 2. In some embodiments, p is 0 or 1. In some embodiments, p is 1 or 2. In some embodiments, p is 0. In some embodiments, p is 1. In some embodiments, p is 2.

In some embodiments, q is 0 or 1. In some embodiments, q is 0. In some embodiments, q is 1. In some embodiments, q is 2.

In some embodiments, the HDAC6 inhibitor has the formula:

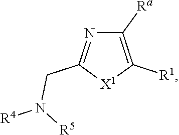

or a salt thereof,
wherein:
   X¹ is S;
   R^{a} is selected from the group consisting of H, halogen, and C₁₋₃ alkyl;
R¹ is

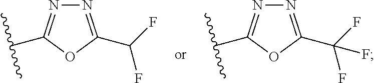 [SMILES: *c1nnc(C(F)F)o1 ; *c1nnc(C(F)(F)F)o1]

R² is selected from the group consisting of alkyl, alkoxy, and cycloalkyl, each of which is optionally substituted;
R³ is H or alkyl;
R⁴ is selected from the group consisting of alkyl, —(SO₂)R², —(SO₂)NR²R³, and —(CO)R²; and
R⁵ is aryl or heteroaryl; or R⁴ and R⁵ together with the atom to which they are attached form a heterocyclyl, each of which is optionally substituted;
In some embodiments, R^{a} is H.

In some embodiments, R¹ is

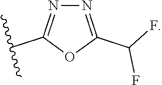 [SMILES: *c1nnc(C(F)F)o1]

In some embodiments, R⁴ is —(SO₂)R².

In some embodiments, —(SO₂)R² is —(SO₂)alkyl, —(SO₂)alkyleneheterocyclyl, —(SO₂)haloalkyl, —(SO₂)haloalkoxy, or —(SO₂)cycloalkyl.

In some embodiments, R⁵ is heteroaryl.

In some embodiments, the heteroaryl is a 5- to 6-membered heteroaryl

In some embodiments, the 5- to 6-membered heteroaryl is selected from the group consisting of

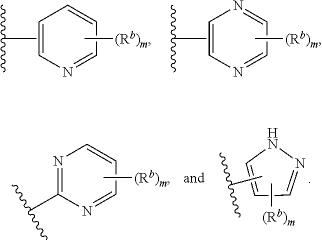

wherein R_{b} is halogen, alkyl, alkoxy, cycloalkyl, —CN, haloalkyl, or haloalkoxy; and m is 0 or 1.

In some embodiments, R^{b} is halogen, haloalkyl, or haloalkoxy. In some embodiments, R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments, the aryl is selected from the group consisting of phenyl, 3-chlorophenyl, 3-chloro-4-fluorophenyl, 3-trifluoromethylphenyl, 3,4-difluorophenyl, and 2,6-difluorophenyl.

In some embodiments, the HDAC6 inhibitor has the Formula (Ik):

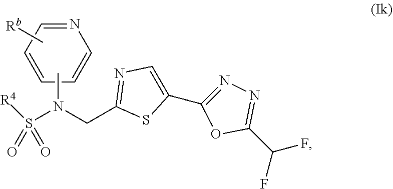

or a salt thereof,
wherein:
   R^{b} is H, halogen, alkyl, cycloalkyl, —CN, haloalkyl, or haloalkoxy; and
   R⁴ is alkyl, alkoxy, haloalkyl, or cycloalkyl, each of which is optionally substituted.

In some embodiments, R^{b} is H, halogen, haloalkyl, or haloalkoxy. In some embodiments, R^{b} is halogen, haloalkyl, or haloalkoxy. In some embodiments, R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments, R⁴ is optionally substituted alkyl or cycloalkyl.

In some embodiments, the HDAC6 inhibitor has the structure:

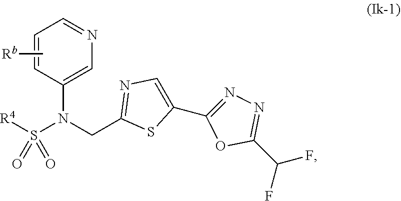

or a salt thereof,
wherein:
   R^{b} is H, halogen, alkyl, cycloalkyl, —CN, haloalkyl, or haloalkoxy; and
   R⁴ is alkyl, alkoxy, haloalkyl, or cycloalkyl, each of which is optionally substituted.

In some embodiments, R^{b} is H, halogen, haloalkyl, or haloalkoxy. In some embodiments, R^{b} is halogen, haloalkyl, or haloalkoxy. In some embodiments, R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments, R⁴ is optionally substituted alkyl or cycloalkyl.

In some embodiments, R⁴ is alkyl.

In some embodiments, the HDAC6 inhibitor has the structure:

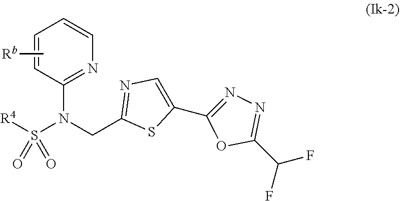

or a salt thereof,
wherein:
   R^{b} is H, halogen, alkyl, cycloalkyl, —CN, haloalkyl, or haloalkoxy; and
   R⁴ is alkyl, alkoxy, haloalkyl, or cycloalkyl, each of which is optionally substituted.

In some embodiments, R^{b} is H, halogen, haloalkyl, or haloalkoxy. In some embodiments, R^{b} is halogen, haloalkyl, or haloalkoxy. In some embodiments, R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments, R⁴ is optionally substituted alkyl.

In some embodiments, the HDAC6 inhibitor is a compound having the formula:

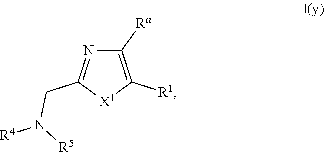

or a salt thereof,
wherein:
X¹ is S;
R^{a} is selected from the group consisting of H, halogen, and C₁₋₃ alkyl;
R¹ is

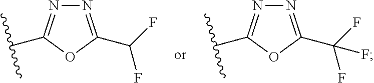 [SMILES: *c1nnc(C(F)F)o1 ; *c1nnc(C(F)(F)F)o1]

R² is selected from the group consisting of alkyl, alkoxy, and cycloalkyl, each of which is optionally substituted;
R³ is H or alkyl;
R⁴ is selected from the group consisting of alkyl, —(SO₂)R², —(SO₂)NR²R³, and —(CO)R²; and
R⁵ is aryl or heteroaryl; or R⁴ and R⁵ together with the atom to which they are attached form a heterocyclyl, each of which is optionally substituted.

In some embodiments of Formula (Iy), R^{a} is H.

In some embodiments of Formula (Iy), R¹ is

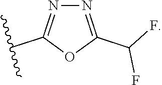 [SMILES: *c1nnc(C(F)F)o1]

In some embodiments of Formula (Iy), R⁴ is —(SO₂)R².

In some embodiments of Formula (Iy), —(SO₂)R² is —(SO₂)alkyl, —(SO₂)alkyleneheterocyclyl, —(SO₂)haloalkyl, —(SO₂)haloalkoxy, or —(SO₂)cycloalkyl.

In some embodiments of Formula (Iy), R⁵ is heteroaryl.

In some embodiments of Formula (Iy), the heteroaryl is a 5- to 6-membered heteroaryl.

In some embodiments of Formula (Iy), the 5- to 6-membered heteroaryl is selected from the group consisting of

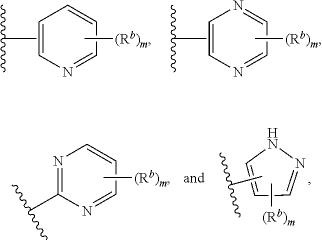

wherein R^{b} is halogen, alkyl, alkoxy, cycloalkyl, —CN, haloalkyl, or haloalkoxy; and m is 0 or 1.

In some embodiments of Formula (Iy), R^{b} is halogen, haloalkyl, or haloalkoxy. In some embodiments, R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments of Formula (Iy), the aryl is selected from the group consisting of phenyl, 3-chlorophenyl, 3-chloro-4-fluorophenyl, 3-trifluoromethylphenyl, 3,4-difluorophenyl, and 2,6-difluorophenyl.

In some embodiments, the HDAC6 inhibitor has the structure:

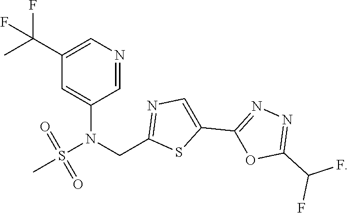 [SMILES: CC(F)(F)c1cncc(N(Cc2ncc(-c3nnc(C(F)F)o3)s2)S(C)(=O)=O)c1]

In some embodiments, the HDAC6 inhibitor has the structure:

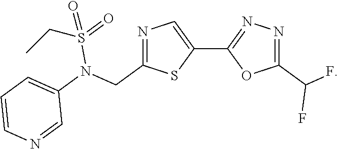 [SMILES: CCS(=O)(=O)N(Cc1ncc(-c2nnc(C(F)F)o2)s1)c1cccnc1]

In some embodiments, the HDAC6 inhibitor has the structure:

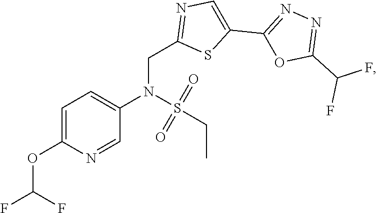 [SMILES: CCS(=O)(=O)N(Cc1ncc(-c2nnc(C(F)F)o2)s1)c1ccc(OC(F)F)nc1]

In some embodiments, the HDAC6 inhibitor has the structure:

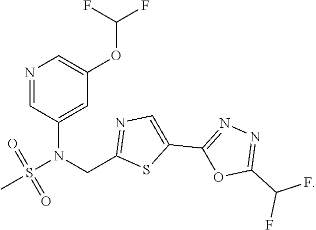 [SMILES: CS(=O)(=O)N(Cc1ncc(-c2nnc(C(F)F)o2)s1)c1cncc(OC(F)F)c1]

In some embodiments, the HDAC6 inhibitor has the structure:

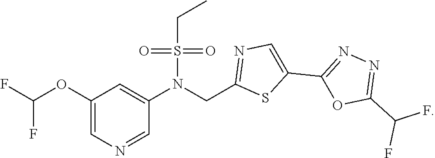 [SMILES: CCS(=O)(=O)N(Cc1ncc(-c2nnc(C(F)F)o2)s1)c1cncc(OC(F)F)c1]

In some embodiments, the HDAC6 inhibitor has the structure:

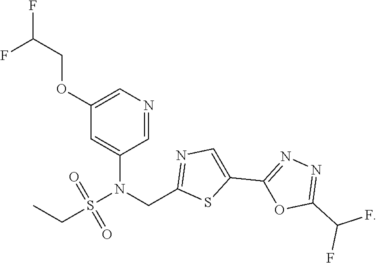 [SMILES: CCS(=O)(=O)N(Cc1ncc(-c2nnc(C(F)F)o2)s1)c1cncc(OCC(F)F)c1]

In some embodiments, the HDAC6 inhibitor has the structure:

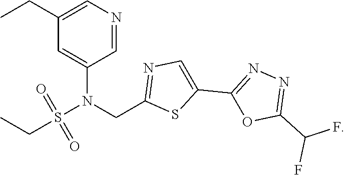 [SMILES: CCc1cncc(N(Cc2ncc(-c3nnc(C(F)F)o3)s2)S(=O)(=O)CC)c1]

In some embodiments, the HDAC6 inhibitor has the structure:

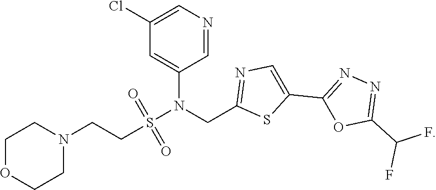 [SMILES: O=S(=O)(CCN1CCOCC1)N(Cc1ncc(-c2nnc(C(F)F)o2)s1)c1cncc(Cl)c1]

In some embodiments, the HDAC6 inhibitor has the structure:

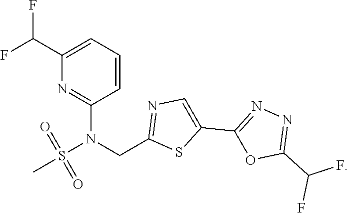 [SMILES: CS(=O)(=O)N(Cc1ncc(-c2nnc(C(F)F)o2)s1)c1cccc(C(F)F)n1]

In some embodiments, the HDAC6 inhibitor has the structure:

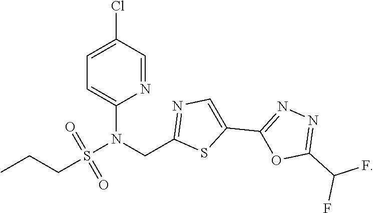 [SMILES: CCCS(=O)(=O)N(Cc1ncc(-c2nnc(C(F)F)o2)s1)c1ccc(Cl)cn1]

In some embodiments, the fluoroalkyl-oxadiazole derivative is TYA-018 or an analog thereof. The structure of TYA-018 is

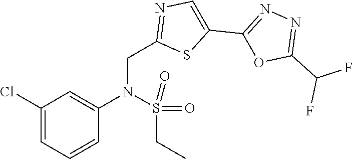 [SMILES: CCS(=O)(=O)N(Cc1ncc(-c2nnc(C(F)F)o2)s1)c1cccc(Cl)c1]

Analogs of TYA-018 include, without limitation, the compounds listed in Table 2.

TABLE 2
Non-limiting Examples of TYA-018 Analogs

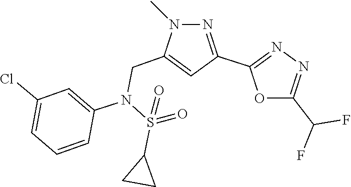 [SMILES: Cn1nc(-c2nnc(C(F)F)o2)cc1CN(c1cccc(Cl)c1)S(=O)(=O)C1CC1]

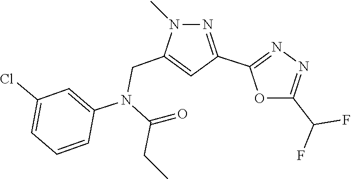 [SMILES: CCC(=O)N(Cc1cc(-c2nnc(C(F)F)o2)nn1C)c1cccc(Cl)c1]

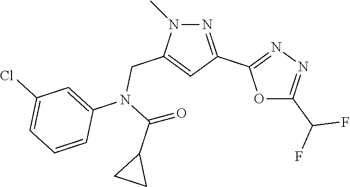 [SMILES: Cn1nc(-c2nnc(C(F)F)o2)cc1CN(C(=O)C1CC1)c1cccc(Cl)c1]

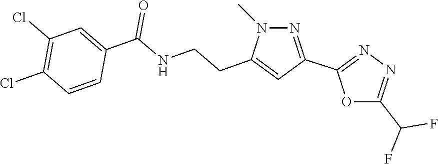 [SMILES: Cn1nc(-c2nnc(C(F)F)o2)cc1CCNC(=O)c1ccc(Cl)c(Cl)c1]

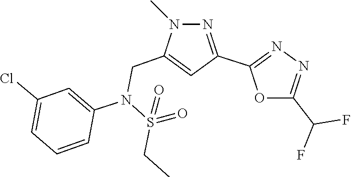 [SMILES: CCS(=O)(=O)N(Cc1cc(-c2nnc(C(F)F)o2)nn1C)c1cccc(Cl)c1]

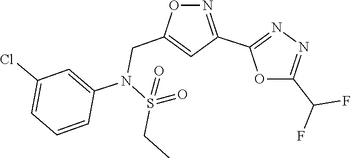 [SMILES: CCS(=O)(=O)N(Cc1cc(-c2nnc(C(F)F)o2)no1)c1cccc(Cl)c1]

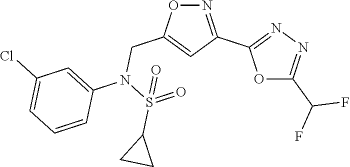 [SMILES: O=S(=O)(C1CC1)N(Cc1cc(-c2nnc(C(F)F)o2)no1)c1cccc(Cl)c1]

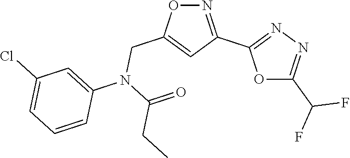 [SMILES: CCC(=O)N(Cc1cc(-c2nnc(C(F)F)o2)no1)c1cccc(Cl)c1]

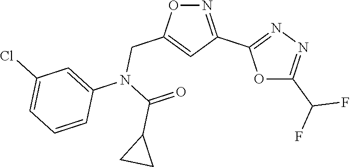 [SMILES: O=C(C1CC1)N(Cc1cc(-c2nnc(C(F)F)o2)no1)c1cccc(Cl)c1]

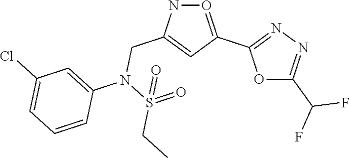

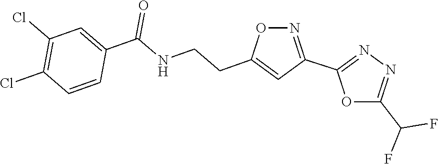 [SMILES: O=C(NCCc1cc(-c2nnc(C(F)F)o2)no1)c1ccc(Cl)c(Cl)c1]

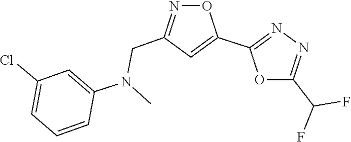 [SMILES: CN(Cc1cc(-c2nnc(C(F)F)o2)on1)c1cccc(Cl)c1]

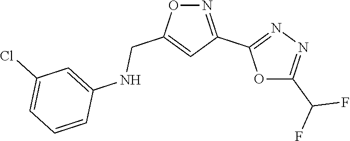 [SMILES: FC(F)c1nnc(-c2cc(CNc3cccc(Cl)c3)on2)o1]

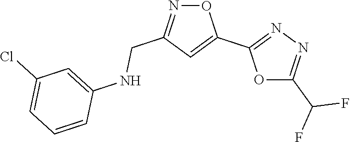 [SMILES: FC(F)c1nnc(-c2cc(CNc3cccc(Cl)c3)no2)o1]

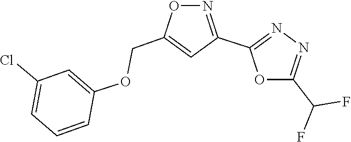 [SMILES: FC(F)c1nnc(-c2cc(COc3cccc(Cl)c3)on2)o1]

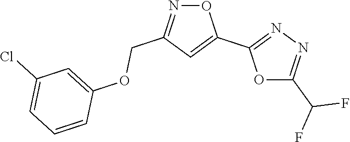 [SMILES: FC(F)c1nnc(-c2cc(COc3cccc(Cl)c3)no2)o1]

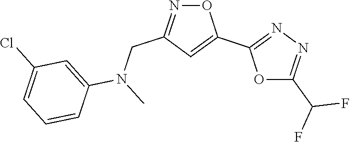 [SMILES: CN(Cc1cc(-c2nnc(C(F)F)o2)on1)c1cccc(Cl)c1]

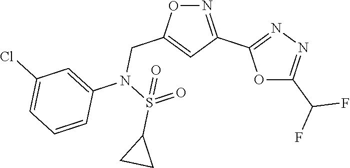 [SMILES: O=S(=O)(C1CC1)N(Cc1cc(-c2nnc(C(F)F)o2)no1)c1cccc(Cl)c1]

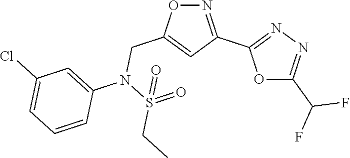 [SMILES: CCS(=O)(=O)N(Cc1cc(-c2nnc(C(F)F)o2)no1)c1cccc(Cl)c1]

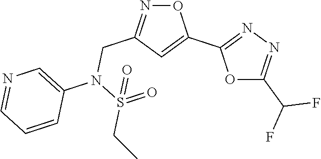 [SMILES: CCS(=O)(=O)N(Cc1cc(-c2nnc(C(F)F)o2)on1)c1cccnc1]

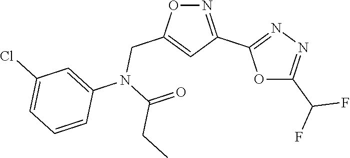 [SMILES: CCC(=O)N(Cc1cc(-c2nnc(C(F)F)o2)no1)c1cccc(Cl)c1]

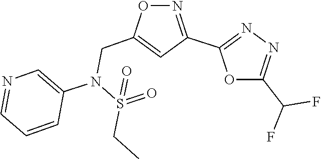 [SMILES: CCS(=O)(=O)N(Cc1cc(-c2nnc(C(F)F)o2)no1)c1cccnc1]

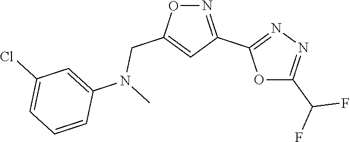 [SMILES: CN(Cc1cc(-c2nnc(C(F)F)o2)no1)c1cccc(Cl)c1]

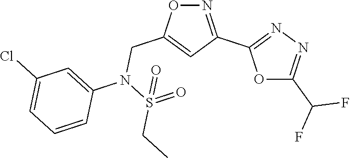 [SMILES: CCS(=O)(=O)N(Cc1cc(-c2nnc(C(F)F)o2)no1)c1cccc(Cl)c1]

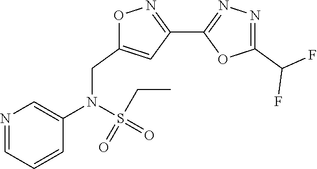 [SMILES: CCS(=O)(=O)N(Cc1cc(-c2nnc(C(F)F)o2)no1)c1cccnc1]

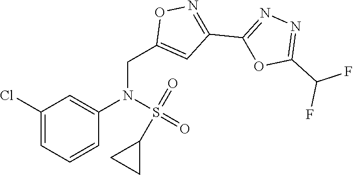 [SMILES: O=S(=O)(C1CC1)N(Cc1cc(-c2nnc(C(F)F)o2)no1)c1cccc(Cl)c1]

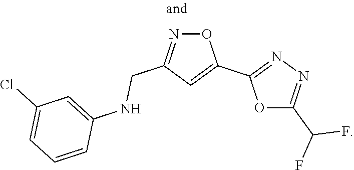 [SMILES: FC(F)c1nnc(-c2cc(CNc3cccc(Cl)c3)no2)o1]

In some embodiments, the HDAC6 inhibitor is a compound selected from the group consisting of:

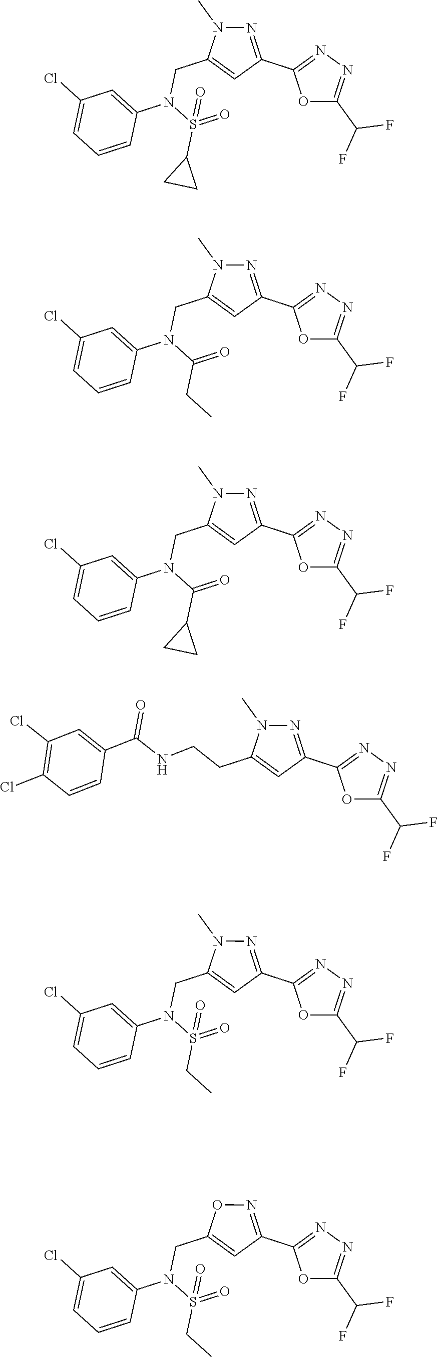 [SMILES: Cn1nc(-c2nnc(C(F)F)o2)cc1CN(c1cccc(Cl)c1)S(=O)(=O)C1CC1 ; CCC(=O)N(Cc1cc(-c2nnc(C(F)F)o2)nn1C)c1cccc(Cl)c1 ; Cn1nc(-c2nnc(C(F)F)o2)cc1CN(C(=O)C1CC1)c1cccc(Cl)c1 ; Cn1nc(-c2nnc(C(F)F)o2)cc1CCNC(=O)c1ccc(Cl)c(Cl)c1 ; CCS(=O)(=O)N(Cc1cc(-c2nnc(C(F)F)o2)nn1C)c1cccc(Cl)c1 ; CCS(=O)(=O)N(Cc1cc(-c2nnc(C(F)F)o2)no1)c1cccc(Cl)c1]

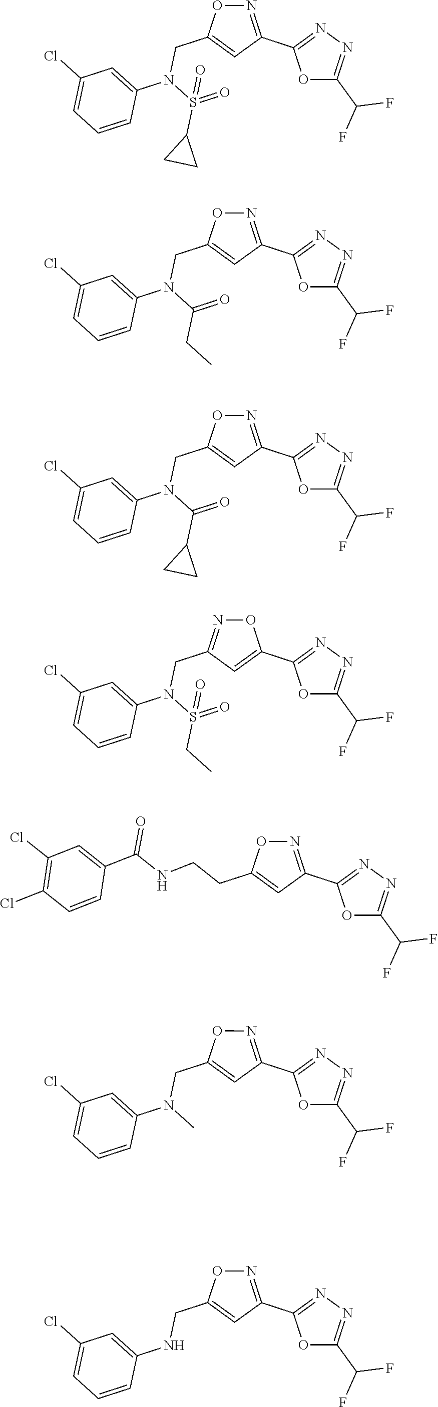

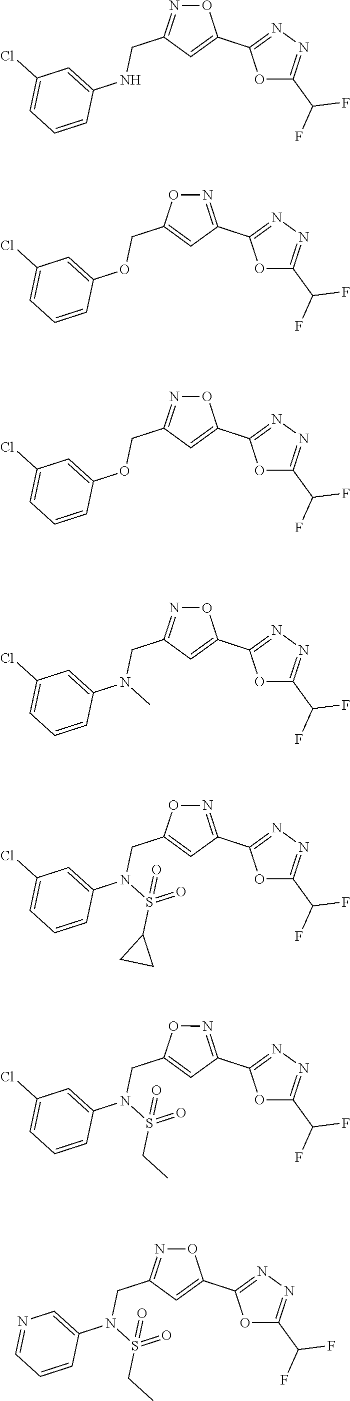

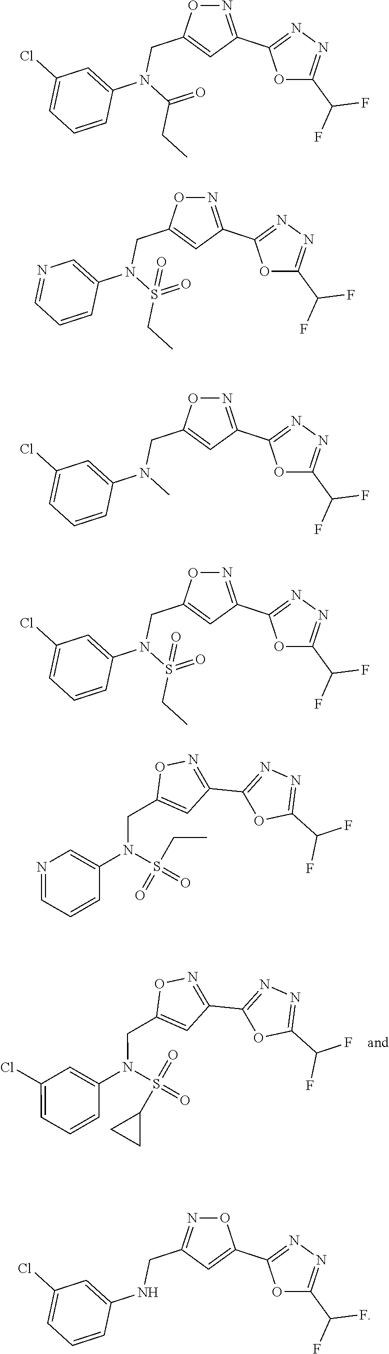

### 5-Fluoronicotinamide Derivatives

In some embodiments, the HDAC6 inhibitor used in the methods described herein is a 5-fluoronicotinamide derivative. Illustrative 5-fluoronicotinamide derivatives that may be used as HDAC6 inhibitors include those described herein and those described in Int′l Pat. Appl. Pub. No. PCT/US2020/054134, published as WO2021067859A1, the content of which is incorporated by reference herein in its entirety. PCT/US2020/054134, published as WO2021067859A1, also describes methods of synthesis of such compounds, which are specifically incorporated by reference herein.

In some embodiments, the HDAC6 inhibitor is a compound of Formula (II):

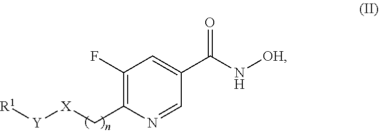

wherein
n is 0 or 1;
X is O, NR⁴, or CR⁴R⁴;
Y is a bond, CR²R³ or S(O)₂;
R¹ is selected from the group consisting of H, amido, carbocyclyl, heterocyclyl, aryl, and heteroaryl;
R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R¹ and R² taken together with the carbon atom to which they are attached form a carbocyclyl or heterocyclyl; or
R² and R³ taken together with the carbon atom to which they are attached form a carbocyclyl or heterocyclyl; and
R⁴ and R^{4′} are each independently selected from the group consisting of H, alkyl, —CO₂-alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R⁴ and R^{4′} taken together with the carbon atom to which they are attached form a carbocyclyl or heterocyclyl;
wherein each alkyl, carbocyclyl, heterocyclyl, aryl, and heteroaryl is independently optionally substituted with one or more substituents selected from the group consisting of halogen, haloalkyl, oxo, hydroxy, alkoxy, —OCH₃, —CO₂CH₃, —C(O)NH(OH),—CH₃, morpholine, and —C(O)N-cyclopropyl.

In some embodiments, the compound of Formula (II) is selected from the group consisting of:

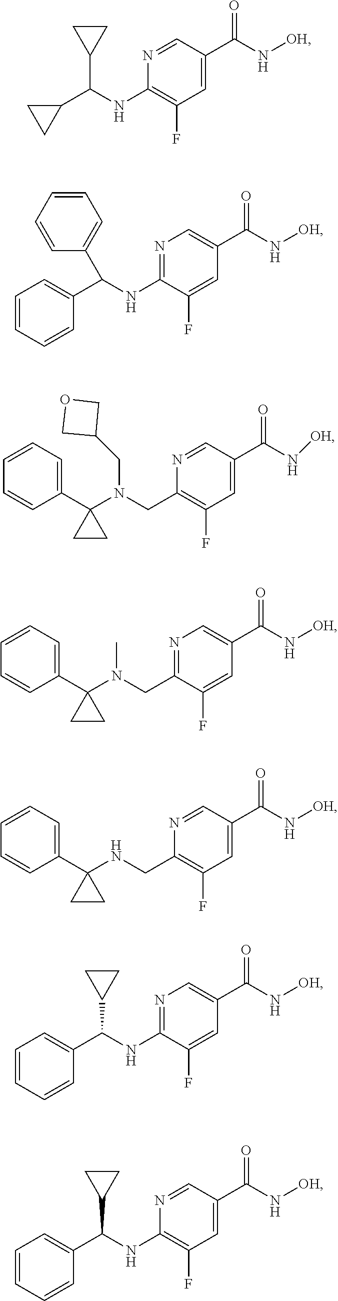

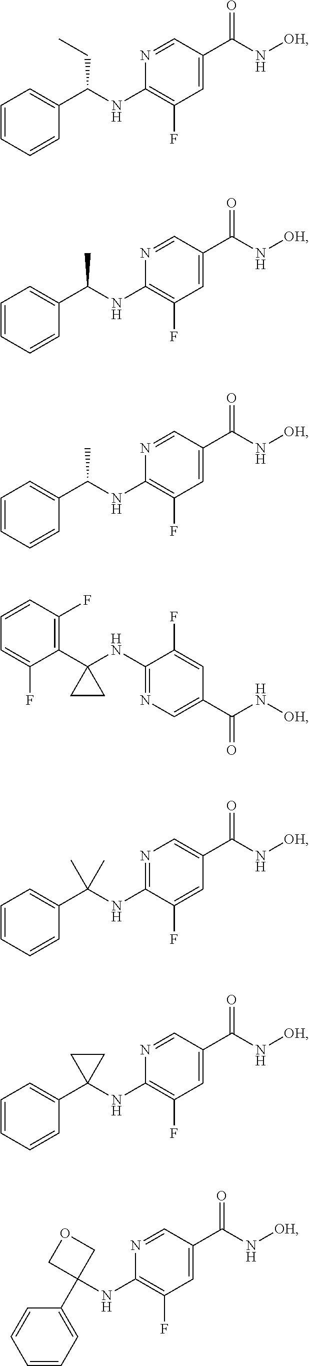

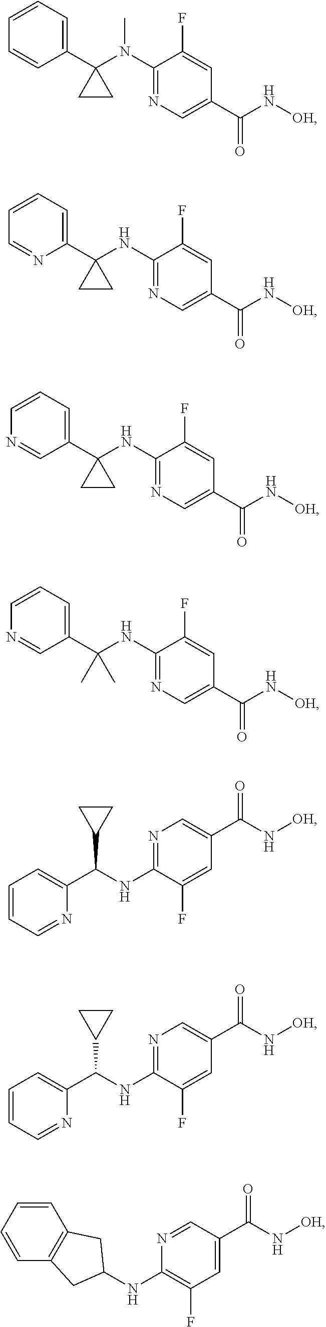

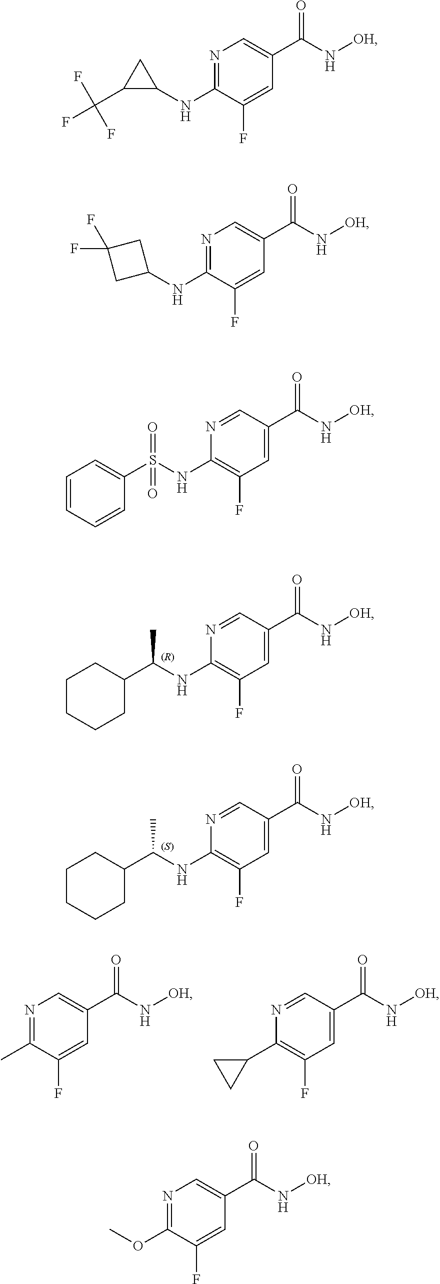

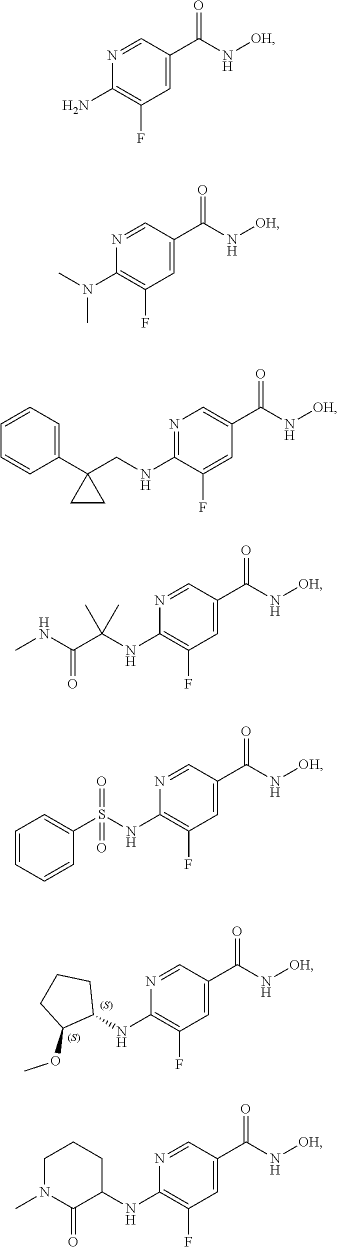

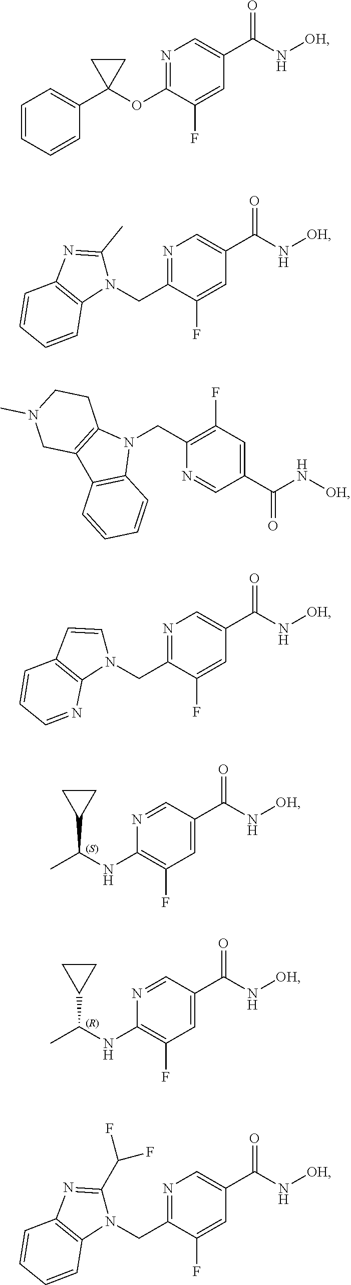

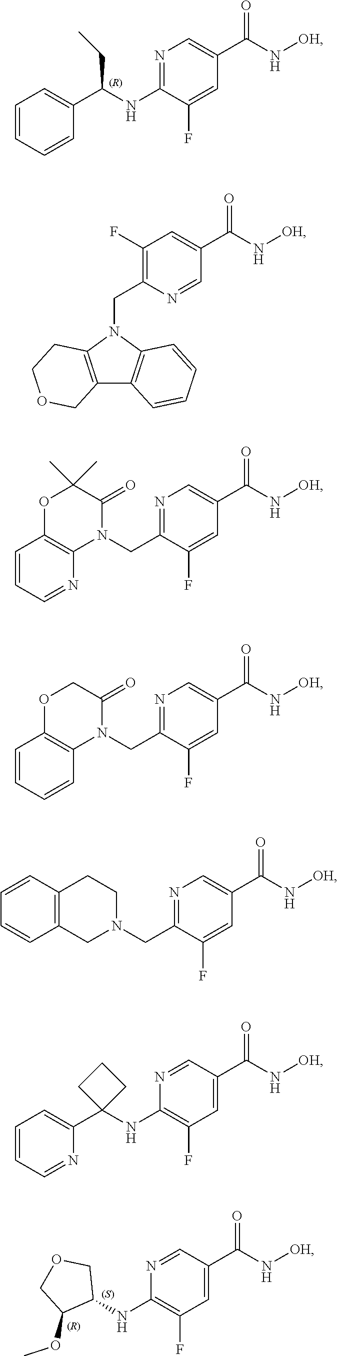

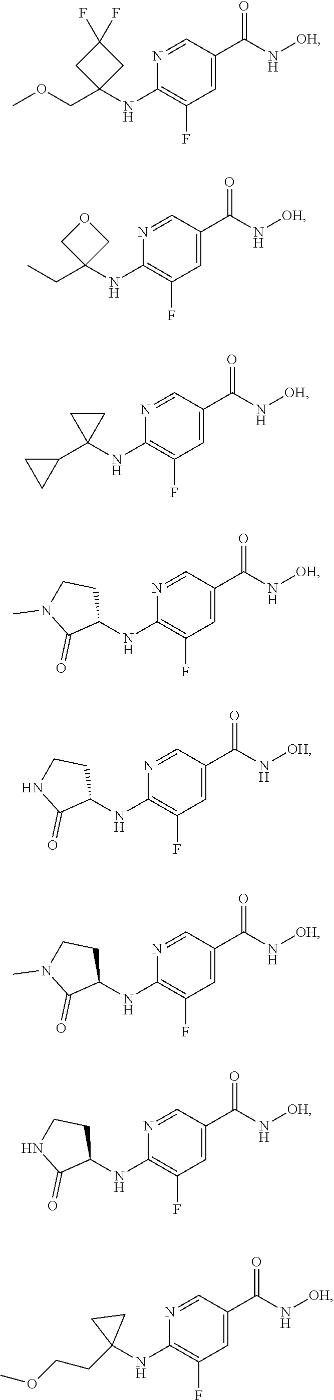

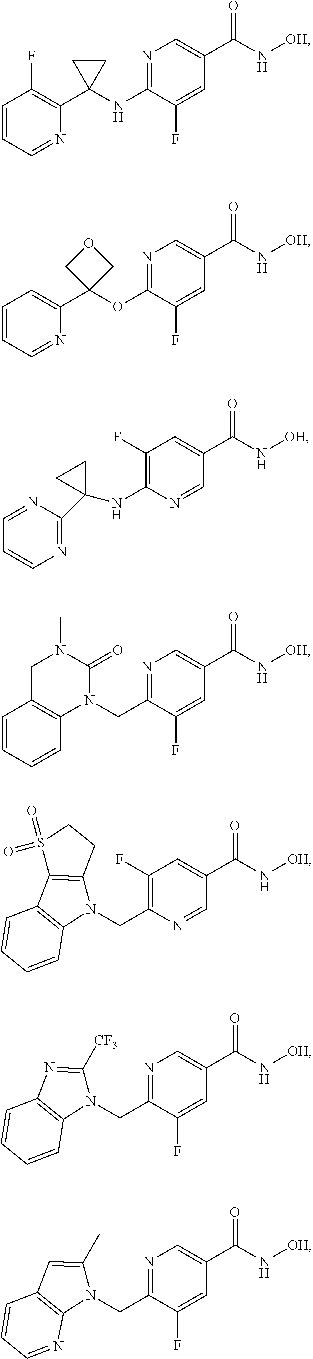

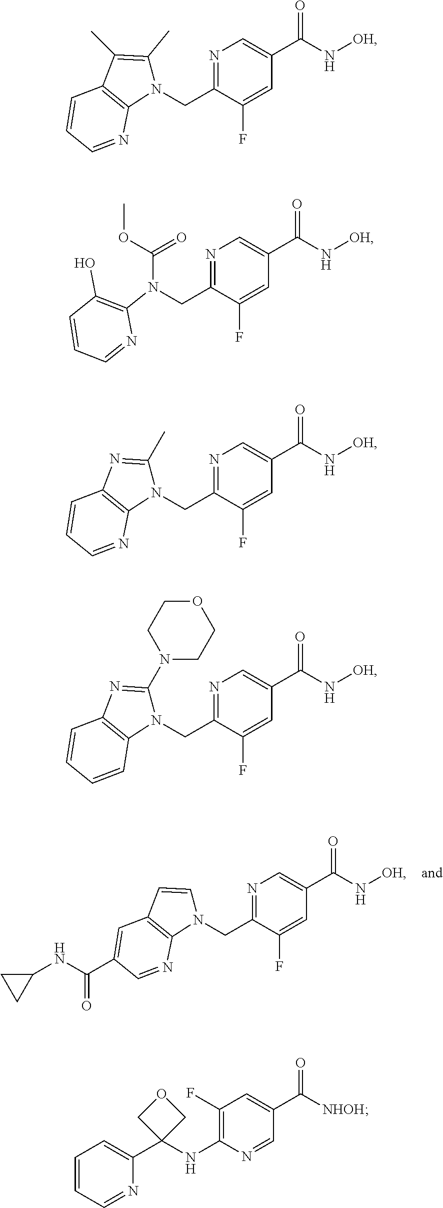

or a salt thereof.

In some embodiments, the compound of Formula (II) is selected from the group consisting of:

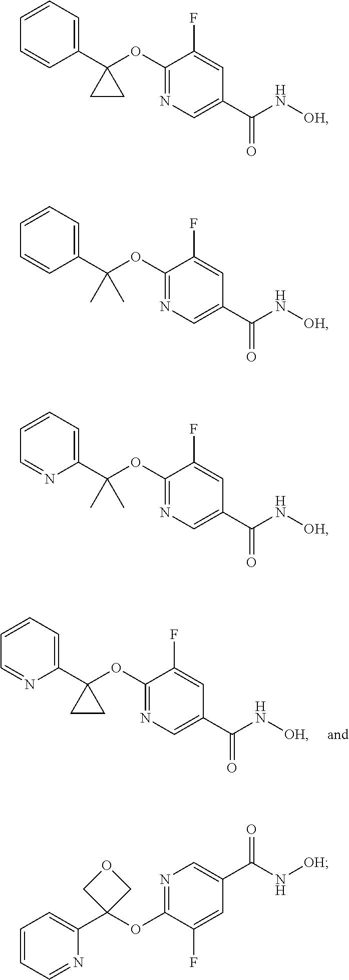 [SMILES: O=C(NO)c1cnc(OC2(c3ccccc3)CC2)c(F)c1 ; CC(C)(Oc1ncc(C(=O)NO)cc1F)c1ccccc1 ; CC(C)(Oc1ncc(C(=O)NO)cc1F)c1ccccn1 ; O=C(NO)c1cnc(OC2(c3ccccn3)CC2)c(F)c1 ; O=C(NO)c1cnc(OC2(c3ccccn3)COC2)c(F)c1]

or a salt thereof.

In some embodiments, the present disclosure provides a compound of Formula (III) or a salt thereof:

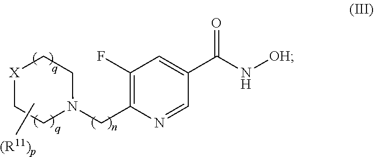

wherein:
n is 0 or 1;
p is 0, 1, 2, 3, or 4;
q is each independently 0, 1, or 2;
X is O, S(O)₂, NR¹², or CHR¹²;
R¹¹ is each independently H, F, alkyl, or oxo; or two adjacent R¹¹ taken together with the carbon atoms to which they are attached form an aryl, heteroaryl, or heterocyclyl ring; or
two non-adjacent R¹¹ taken together with the atoms to which they are attached form a carbocyclyl or heterocyclyl ring;
R¹² is selected from the group consisting of alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R¹¹ and R¹² taken together with the carbon and/or nitrogen atoms to which they are attached form an aryl, heteroaryl ring, or heterocyclyl ring; and wherein each alkyl, carbocyclyl, heterocyclyl, aryl, and heteroaryl is independently optionally substituted with one or more substituents selected from the group consisting of halogen, oxo, hydroxy, alkoxy, —OCH₃, —CO₂CH₃, and —CH₃.

In some embodiments, the compound of Formula (III) is selected from the group consisting of:

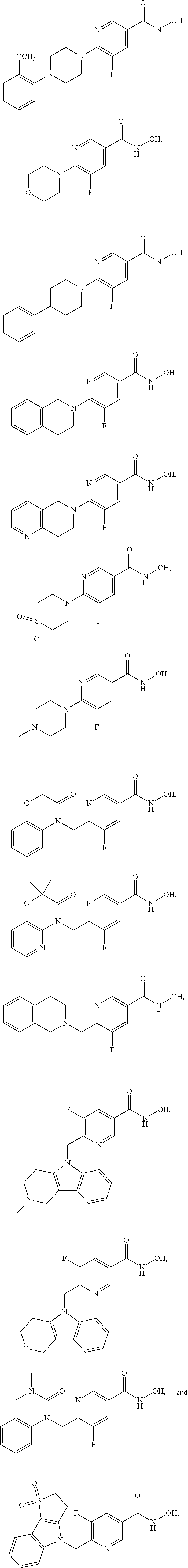

or a salt thereof.

In some embodiments, the HDAC6 inhibitor is a compound having one of the following structures:

| ID | Structure |
|---|---|
| 1 | 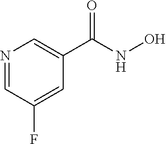 [SMILES: O=C(NO)c1cncc(F)c1] |
| 2 | 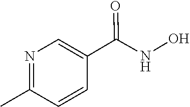 |
| II-1 | 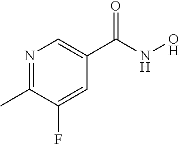 |
| II-2 | 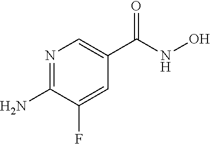 [SMILES: Nc1ncc(C(=O)NO)cc1F] |
| II-3 | 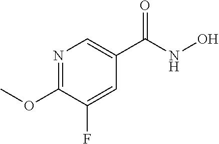 |
| II-4 | 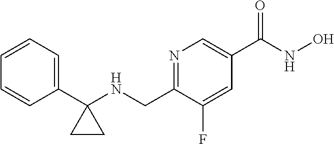 [SMILES: O=C(NO)c1cnc(CNC2(c3ccccc3)CC2)c(F)c1] |
| II-5 | 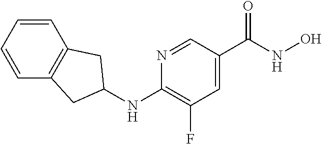 [SMILES: O=C(NO)c1cnc(NC2Cc3ccccc3C2)c(F)c1] |
| II-6 | 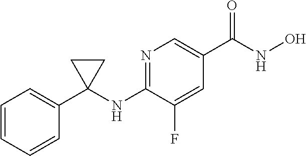 [SMILES: O=C(NO)c1cnc(NC2(c3ccccc3)CC2)c(F)c1] |
| III-1 | 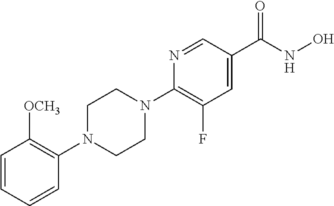 [SMILES: COc1ccccc1N1CCN(c2ncc(C(=O)NO)cc2F)CC1] |
| II-7A | 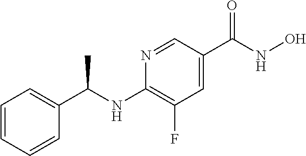 [SMILES: C[C@@H](Nc1ncc(C(=O)NO)cc1F)c1ccccc1] |
| II-7B | 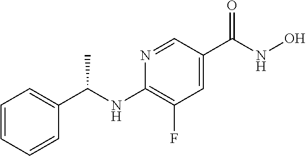 [SMILES: C[C@H](Nc1ncc(C(=O)NO)cc1F)c1ccccc1] |
| II-8B | 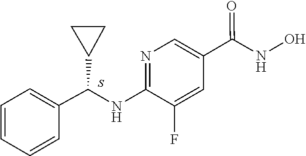 |
| II-9A | 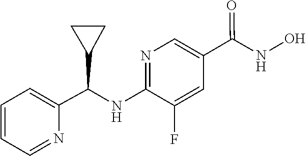 [SMILES: O=C(NO)c1cnc(N[C@@H](c2ccccn2)C2CC2)c(F)c1] |
| II-9B | 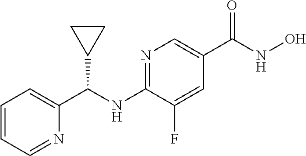 [SMILES: O=C(NO)c1cnc(N[C@H](c2ccccn2)C2CC2)c(F)c1] |
| II-10 | 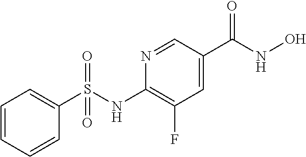 [SMILES: O=C(NO)c1cnc(NS(=O)(=O)c2ccccc2)c(F)c1] |
| III-2 | 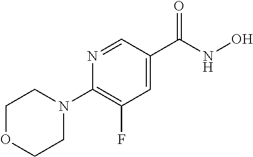 [SMILES: O=C(NO)c1cnc(N2CCOCC2)c(F)c1] |
| II-11 | 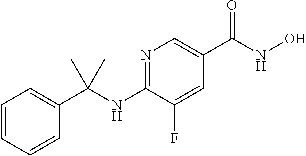 [SMILES: CC(C)(Nc1ncc(C(=O)NO)cc1F)c1ccccc1] |
| III-3 | 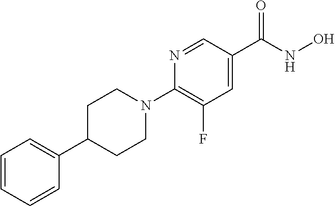 [SMILES: O=C(NO)c1cnc(N2CCC(c3ccccc3)CC2)c(F)c1] |
| III-4 | 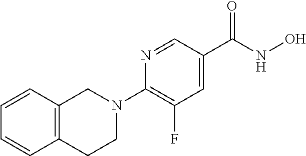 [SMILES: O=C(NO)c1cnc(N2CCc3ccccc3C2)c(F)c1] |
| II-12B | 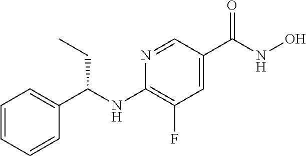 [SMILES: CC[C@H](Nc1ncc(C(=O)NO)cc1F)c1ccccc1] |
| II-8A | 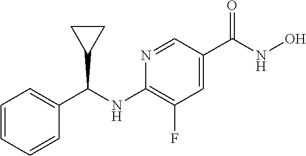 [SMILES: O=C(NO)c1cnc(N[C@@H](c2ccccc2)C2CC2)c(F)c1] |
| II-13 | 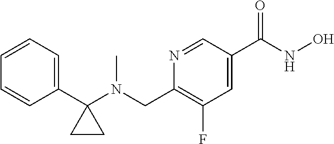 [SMILES: CN(Cc1ncc(C(=O)NO)cc1F)C1(c2ccccc2)CC1] |
| II-14 | 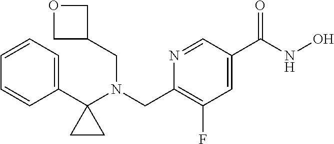 [SMILES: O=C(NO)c1cnc(CN(CC2COC2)C2(c3ccccc3)CC2)c(F)c1] |
| II-15 | 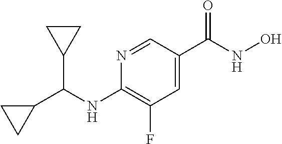 [SMILES: O=C(NO)c1cnc(NC(C2CC2)C2CC2)c(F)c1] |
| II-16 | 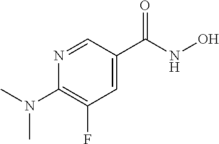 |
| III-5 | 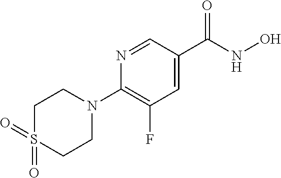 [SMILES: O=C(NO)c1cnc(N2CCS(=O)(=O)CC2)c(F)c1] |
| III-6 | 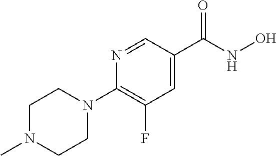 [SMILES: CN1CCN(c2ncc(C(=O)NO)cc2F)CC1] |
| 3 | 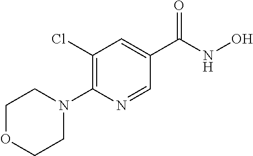 [SMILES: O=C(NO)c1cnc(N2CCOCC2)c(Cl)c1] |
| II-17 | 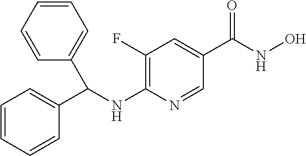 [SMILES: O=C(NO)c1cnc(NC(c2ccccc2)c2ccccc2)c(F)c1] |
| 4 | 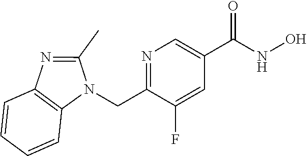 [SMILES: Cc1nc2ccccc2n1Cc1ncc(C(=O)NO)cc1F] |
| III-7 | 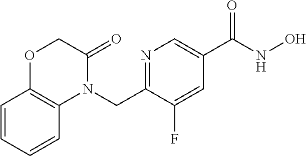 [SMILES: O=C(NO)c1cnc(CN2C(=O)COc3ccccc32)c(F)c1] |
| III-8 | 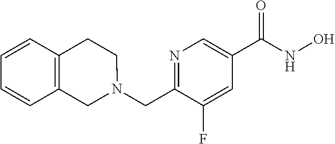 [SMILES: O=C(NO)c1cnc(CN2CCc3ccccc3C2)c(F)c1] |
| II-18 | 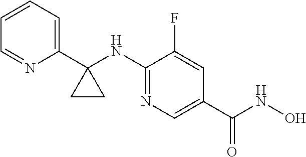 [SMILES: O=C(NO)c1cnc(NC2(c3ccccn3)CC2)c(F)c1] |
| II-19 | 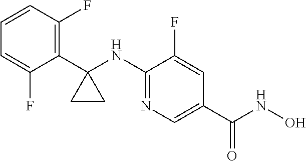 [SMILES: O=C(NO)c1cnc(NC2(c3c(F)cccc3F)CC2)c(F)c1] |
| II-20 | 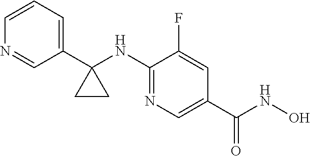 [SMILES: O=C(NO)c1cnc(NC2(c3cccnc3)CC2)c(F)c1] |
| II-21 | 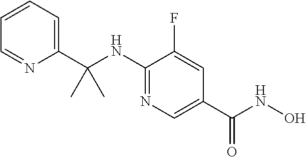 [SMILES: CC(C)(Nc1ncc(C(=O)NO)cc1F)c1ccccn1] |
| II-22 | 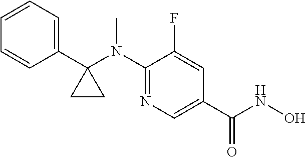 [SMILES: CN(c1ncc(C(=O)NO)cc1F)C1(c2ccccc2)CC1] |
| II-23 | 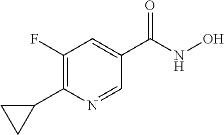 [SMILES: O=C(NO)c1cnc(C2CC2)c(F)c1] |
| II-25 | 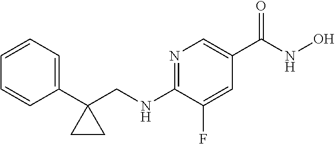 [SMILES: O=C(NO)c1cnc(NCC2(c3ccccc3)CC2)c(F)c1] |
| III-9 | 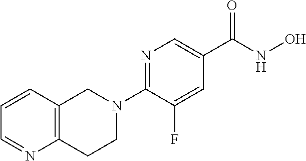 [SMILES: O=C(NO)c1cnc(N2CCc3ncccc3C2)c(F)c1] |
| I-1a | 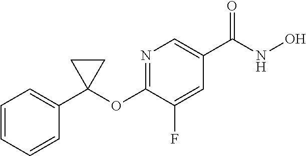 [SMILES: O=C(NO)c1cnc(OC2(c3ccccc3)CC2)c(F)c1] |
| III-10 | 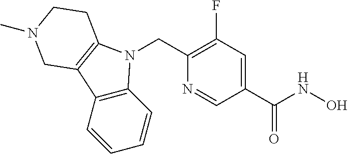 [SMILES: CN1CCc2c(c3ccccc3n2Cc2ncc(C(=O)NO)cc2F)C1] |
| 5 | 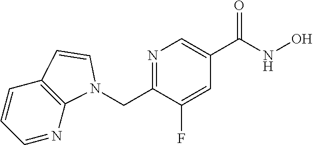 [SMILES: O=C(NO)c1cnc(Cn2ccc3cccnc32)c(F)c1] |
| II-26A | 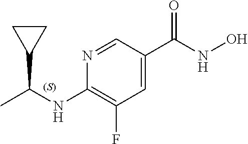 |
| II-26B | 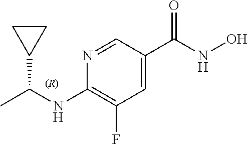 |
| 6 | 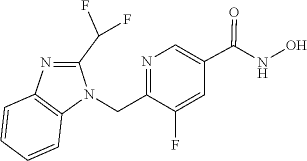 [SMILES: O=C(NO)c1cnc(Cn2c(C(F)F)nc3ccccc32)c(F)c1] |
| II-27 | 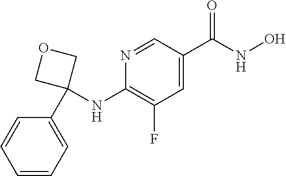 [SMILES: O=C(NO)c1cnc(NC2(c3ccccc3)COC2)c(F)c1] |
| II-28A | 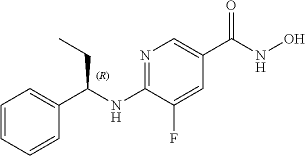 |
| II-29 | 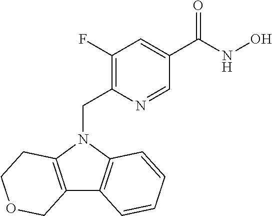 [SMILES: O=C(NO)c1cnc(Cn2c3c(c4ccccc42)COCC3)c(F)c1] |
| II-30A | 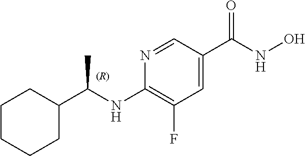 |
| II-30B | 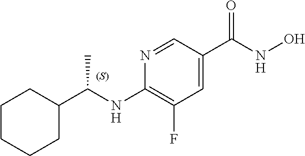 |
| II-31 | 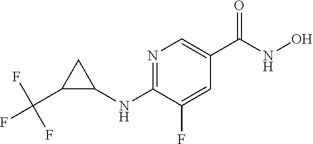 [SMILES: O=C(NO)c1cnc(NC2CC2C(F)(F)F)c(F)c1] |
| II-32 | 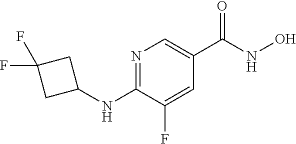 [SMILES: O=C(NO)c1cnc(NC2CC(F)(F)C2)c(F)c1] |
| II-33 | 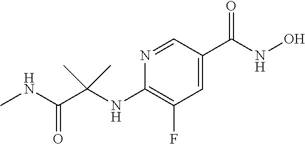 [SMILES: CNC(=O)C(C)(C)Nc1ncc(C(=O)NO)cc1F] |
| II-34 | 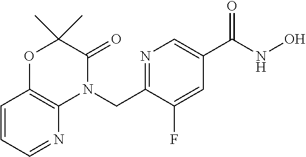 [SMILES: CC1(C)Oc2cccnc2N(Cc2ncc(C(=O)NO)cc2F)C1=O] |
| II-35 | 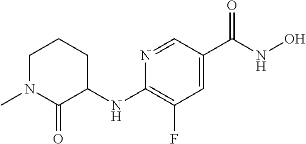 [SMILES: CN1CCCC(Nc2ncc(C(=O)NO)cc2F)C1=O] |
| II-36 | 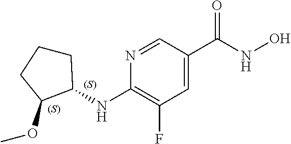 |
| II-37 | 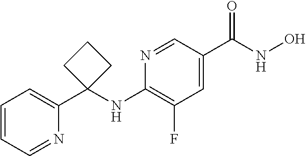 [SMILES: O=C(NO)c1cnc(NC2(c3ccccn3)CCC2)c(F)c1] |
| II-43 | 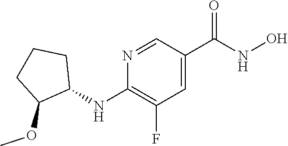 [SMILES: CO[C@H]1CCC[C@@H]1Nc1ncc(C(=O)NO)cc1F] |
| II-38 | 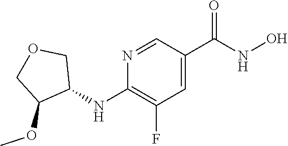 [SMILES: CO[C@H]1COC[C@@H]1Nc1ncc(C(=O)NO)cc1F] |
| II-39 | 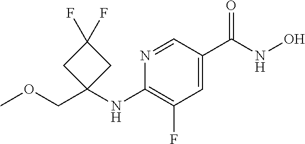 [SMILES: COCC1(Nc2ncc(C(=O)NO)cc2F)CC(F)(F)C1] |
| II-40 | 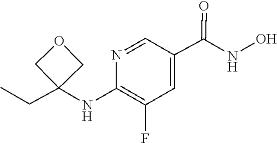 [SMILES: CCC1(Nc2ncc(C(=O)NO)cc2F)COC1] |
| II-41 | 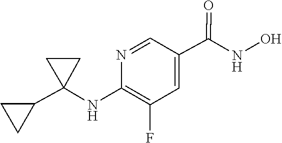 [SMILES: O=C(NO)c1cnc(NC2(C3CC3)CC2)c(F)c1] |
| II-42A | 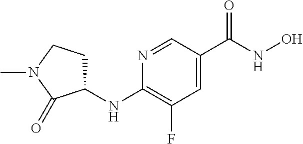 [SMILES: CN1CC[C@H](Nc2ncc(C(=O)NO)cc2F)C1=O] |
| II-43A | 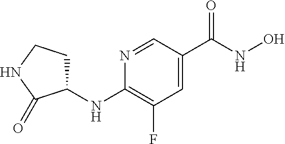 [SMILES: O=C(NO)c1cnc(N[C@H]2CCNC2=O)c(F)c1] |
| II-42B | 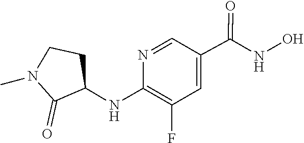 [SMILES: CN1CC[C@@H](Nc2ncc(C(=O)NO)cc2F)C1=O] |
| II-43B | 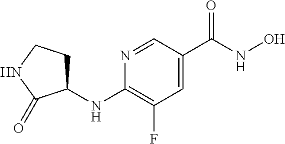 [SMILES: O=C(NO)c1cnc(N[C@@H]2CCNC2=O)c(F)c1] |
| II-44 | 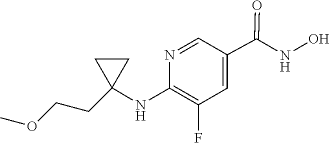 [SMILES: COCCC1(Nc2ncc(C(=O)NO)cc2F)CC1] |
| II-45 | 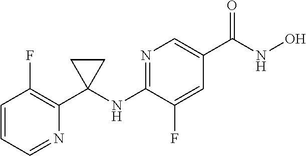 [SMILES: O=C(NO)c1cnc(NC2(c3ncccc3F)CC2)c(F)c1] |
| I-2a | 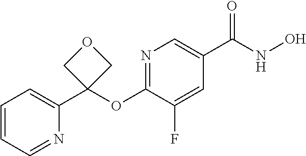 [SMILES: O=C(NO)c1cnc(OC2(c3ccccn3)COC2)c(F)c1] |
| II-46 | 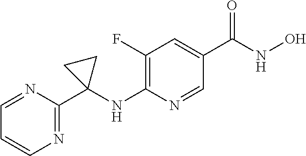 [SMILES: O=C(NO)c1cnc(NC2(c3ncccn3)CC2)c(F)c1] |
| II-47 | 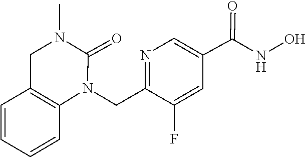 [SMILES: CN1Cc2ccccc2N(Cc2ncc(C(=O)NO)cc2F)C1=O] |
| II-48 | 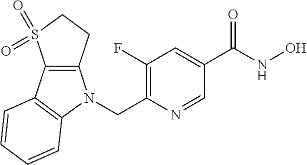 [SMILES: O=C(NO)c1cnc(Cn2c3c(c4ccccc42)S(=O)(=O)CC3)c(F)c1] |
| II-49 | 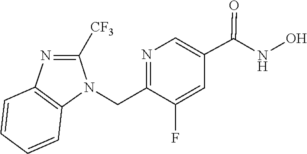 [SMILES: O=C(NO)c1cnc(Cn2c(C(F)(F)F)nc3ccccc32)c(F)c1] |
| II-50 | 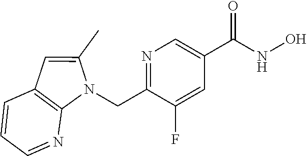 [SMILES: Cc1cc2cccnc2n1Cc1ncc(C(=O)NO)cc1F] |
| II-51 | 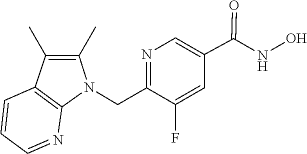 [SMILES: Cc1c(C)n(Cc2ncc(C(=O)NO)cc2F)c2ncccc12] |
| II-52 | 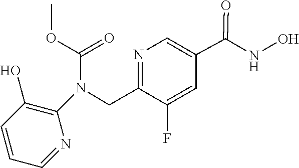 [SMILES: COC(=O)N(Cc1ncc(C(=O)NO)cc1F)c1ncccc1O] |
| II-53 | 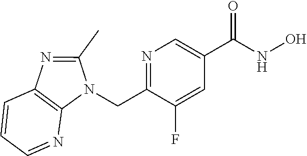 [SMILES: Cc1nc2cccnc2n1Cc1ncc(C(=O)NO)cc1F] |
| II-54 | 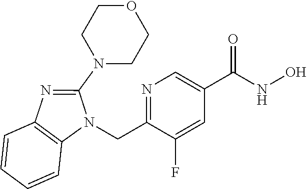 [SMILES: O=C(NO)c1cnc(Cn2c(N3CCOCC3)nc3ccccc32)c(F)c1] |
| II-55 | 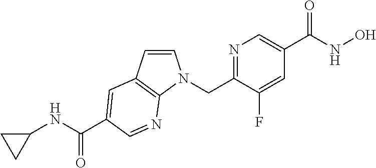 [SMILES: O=C(NO)c1cnc(Cn2ccc3cc(C(=O)NC4CC4)cnc32)c(F)c1] |
| II-56 | 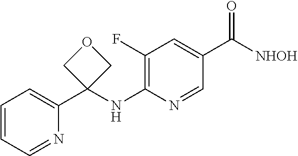 |

Combination with Additional Compounds

In some embodiments, the selective HDAC6 inhibitors of the present technology can be used in combination with one or more additional compounds in the methods described herein. As shown in the working examples, a combination of selective HDAC6 inhibitors and additional compounds may further increase yield of viral production from cell culture (e.g., HEK293 cell culture) in the methods described herein. In some embodiments, the additional compounds comprise one or more selected from the group consisting of a caspase (e.g., caspases-3 and/or caspase-7) inhibitor, a stimulator of interferon genes (STING) inhibitor, and a pan-HDAC inhibitor. In some embodiments, the caspase inhibitor comprises Ac-DEVD-CHO (N—Ac-Asp-Glu-Val-Asp-CHO). In some embodiments, the STING inhibitor comprises SN-011. In some embodiments, the pan-HDAC inhibitor comprises Ricolinostat. In some embodiments, the caspase inhibitor, the STING inhibitor, and/or the pan-HDAC inhibitor can be used in cell culture in a concentration range of 1-20 μM, 1-15 μM, 1-10 μM, or 1-5 μM, e.g., about 1.25 μM, about 2.5 μM, about 3.75 μM, or about 5 μM.

### Transgenes

The transgenes described herein are non-limiting. Any transgene may be used in the viral vectors produced in accordance with the methods described herein.

A transgene can be a gene or nucleotide sequence that encodes a product, or functional fragment thereof. A product can be, for example, a polypeptide or a non-coding nucleotide. By non-coding nucleotide, it is meant that the sequence transcribed from the transgene or nucleotide sequence is not translated into a polypeptide. In some embodiments, the product encoded by the transgene or nucleotide operably linked to an enhancer described herein is a non-coding polynucleotide. A non-coding polynucleotide can be an RNA, such as for example a microRNA (miRNA or mIR), short hairpin RNA (shRNA), long non-coding RNA (lnRNA), and/or a short interfering RNA (siRNA). In some embodiments, the transgene encodes a product natively expressed by a cardiac cell, e.g., a cardiomyocyte. In some embodiments, the transgene encodes a product natively expressed in a cell type other than a cardiac cell.

In some embodiments, the transgene encodes a polypeptide. In some embodiments, the transgene encodes a non-coding polynucleotide such as, for example, a microRNA (miRNA or mIR).

In some embodiments, the transgene comprises a sequence encoding a product selected from cadherins, connexins, Cx43, growth factors such as fibroblast growth factor (FGF)-2 and transforming growth factor-β, cytokines such as interleukin (IL)-1β and the IL-6 family, leukemia inhibitory factor, cardiotrophin-1, cardiogenic transcription factors, insulin-like growth factor, GATA4, MEF2C, TBX5, ESRRG, MESP1, MYOCD, ZFPM2, HAND2, miR-1, miR-133, Oct4, Sox2, Klf4, c-Myc, SRF, SMARCD3, Nkx2-5, Akt, PKB, Baf60c, BMP4, miR-208, and miR-499.

In some embodiments, the transgene encodes a genome-editing endonuclease (optionally with a guide RNA, single-guide RNA, and/or repair template) that replaces or repairs, e.g., a non-functional cardiac protein into a functional cardiac protein. In some embodiments, the transgene encodes a cardiac troponin T; a cardiac sarcomeric protein; β-myosin heavy chain; myosin ventricular essential light chain 1; myosin ventricular regulatory light chain 2; cardiac a-actin; a-tropomyosin; cardiac troponin I; cardiac myosin binding protein C; four-and-a-half LIM protein 1; titin; 5′-AMP-activated protein kinase subunit gamma-2; troponin I type 3, myosin light chain 2, actin alpha cardiac muscle 1; cardiac LIM protein; caveolin 3 (CAV3); galactosidase alpha (GLA); lysosomal-associated membrane protein 2 (LAMP2); mitochondrial transfer RNA glycine (MTTG); mitochondrial transfer RNA isoleucine (MTTI); mitochondrial transfer RNA lysine (MTTK); mitochondrial transfer RNA glutamine (MTTQ); myosin light chain 3 (MYL3); troponin C (TNNC1); transthyretin (TTR); sarcoendoplasmic reticulum calcium-ATPase 2a (SERCA2a); stromal-derived factor-1 (SDF-1); adenylate cyclase-6 (AC6); beta-ARKct (β-adrenergic receptor kinase C terminus); fibroblast growth factor (FGF); platelet-derived growth factor (PDGF); vascular endothelial growth factor (VEGF); hepatocyte growth factor; hypoxia inducible growth factor; thymosin beta 4 (TMSB4X); nitric oxide synthase-3 (NOS3); unocartin 3 (UCN3); melusin; apoplipoprotein-E (ApoE); superoxide dismutase (SOD); and S100A1 (a small calcium binding protein; see, e.g., Ritterhoff and Most (2012) Gene Ther. 19:613; Kraus et al. (2009) Mol. Cell. Cardiol. 47:445).

In some embodiments, the transgene comprises a sequence encoding a product selected from vascular endothelial growth factor (VEGF), a VEGF isoform, VEGF-A, VEGF-B, VEGF-C, VEGF-D, VEGF-D^{dNdC}, VEGF-A₁₁₆A, VEGF-A₁₆₅, VEGF-A₁₂₁, VEGF-2, placenta growth factor (PIGF), fibroblast growth factor 4 (FGF-4), human growth factor (HGF), human granulocyte colony-stimulating factor (hGCSF), and hypoxia inducible factor 1α (HIF-1α).

In some embodiments, the transgene comprises a sequence encoding a product selected from SERCA2a, stromal cell-derived factor-1 (SDF-1), adenylyl cyclase type 6, S100A1, miRNA-17-92, miR-302-367, anti-miR-29a, anti-miR-30a, antimiR-141, cyclin A2, cyclin-dependent kinase 2, Tbx20, miRNA-590, miRNA-199, anti-sense oligonucleotide against Lp(a), interfering RNA against PCSK9, anti-sense oligonucleotide against apolipoprotein C-III, lipoprotein lipases^{S447X}, anti-sense oligonucleotide against apolipoprotein B, anti-sense oligonucleotide against c-myc, and E2F oligonucleotide decoy.

In some embodiments, the transgene encodes a gene product whose expression complements a defect in a gene responsible for a genetic disorder. The disclosure polynucleotides encoding one or more of the following—e.g., for use, without limitation, in the disorder indicated in parentheses, or for other disorders caused by each: TAZ (Barth syndrome); FXN (Freidrich's Ataxia); CASQ2 (CPVT); FBN1 (Marfan); RAF1 and SOS1s (Noonan); SCN5A (Brugada); KCNQ1 and KCNH2s (Long QT Syndrome); DMPK (Myotonic Dystrophy 1); LMNA (Limb Girdle Dystrophy Type 1B); JUP (Naxos); TGFBR2 (Loeys-Dietz); EMD (X-Linked EDMD); and ELN (SV Aortic Stenosis). In some embodiments, a polynucleotide encodes one or more of: cardiac troponin T (TNNT2); BAG family molecular chaperone regulator 3 (BAG3); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (TTN); myosin, light chain 2 (MYL2); actin, alpha cardiac muscle 1 (ACTC1); potassium voltage-gated channel, KQT-like subfamily, member 1 (KCNQ1); myocyte enhancer factor 2c (MEF2C); and cardiac LIM protein (CSRP3).

In some embodiments, the transgene comprises a nucleotide sequence encoding a protein selected from DWORF, junctophilin (e.g., JPH2), BAG family molecular chaperone regulator 3 (BAG3), phospholamban (PLN), alpha-crystallin B chain (CRYAB), LMNA (such as Lamin A and Lamin C isoforms), troponin I type 3 (TNNI3), lysosomal-associated membrane protein 2 (LAMP2, such as LAMP2a, LAMP2b and LAMP2c isoforms), desmoplakin (DSP, such as DPI and DPII isoforms), desmoglein 2 (DSG2), junction plakoglobin (JUP), and plakophilin-2 (PKP2). In some embodiments, the transgene comprises a nucleotide sequence encoding a human protein. In some embodiments, the transgene comprises a human nucleotide sequence (a human DNA sequence). In some embodiments, the transgene comprises a DNA sequence that has been codon-optimized. In some embodiments, the transgene comprises a nucleotide sequence encoding a wild-type protein, or a functionally active fragment thereof.

In some embodiments, the transgene comprises a polynucleotide sequence encoding a DWORF polypeptide.

In some embodiments, the transgene comprises a polynucleotide sequence encoding a junctophilin 2 (JPH2) polypeptide. In some embodiments, the transgene comprises a polynucleotide sequence encoding a full-length JPH2 polypeptide. In some embodiments, the transgene comprises a polynucleotide sequence encoding an N-terminal fragment of the JPH2 polypeptide. In some embodiments, the transgene comprises a polynucleotide sequence encoding an N-terminal fragment of the JPH2 polypeptide, which retains the JPH2 activity.

In some embodiments, the transgene comprises a polynucleotide sequence encoding a BAG3 polypeptide.

In some embodiments, the transgene comprises a polynucleotide sequence encoding a CRYAB polypeptide.

In some embodiments, the transgene comprises a polynucleotide sequence encoding a LMNA polypeptide. In some embodiments, the transgene comprises a polynucleotide sequence encoding the LaminA isoform of LMNA. In some embodiments, the transgene comprises a polynucleotide sequence encoding the LaminC isoform of LMNA.

In some embodiments, the transgene comprises a polynucleotide sequence encoding a TNNI3 polypeptide.

In some embodiments, the transgene comprises a polynucleotide sequence encoding a PLN polypeptide.

In some embodiments, the transgene comprises a polynucleotide sequence encoding a LAMP2 polypeptide. In some embodiments, the transgene comprises a polynucleotide sequence encoding the LAMP2a isoform. In some embodiments, the transgene comprises a polynucleotide sequence encoding the LAMP2b isoform. In some embodiments, the transgene comprises a polynucleotide sequence encoding the LAMP2c isoform.

In some embodiments, the transgene comprises a polynucleotide sequence encoding a DSP polypeptide. In some embodiments, the transgene comprises a polynucleotide sequence encoding the DPI isoform of DSP. In some embodiments, the transgene comprises a polynucleotide sequence encoding the DPII isoform of DSP.

In some embodiments, the transgene comprises a polynucleotide sequence encoding a DSG2 polypeptide.

In some embodiments, the transgene comprises a polynucleotide sequence encoding a JUP polypeptide.

### Examples

The invention is further illustrated by the following examples. The examples below are non-limiting are merely representative of various aspects of the invention.

### Example 1. Synthesis, Biochemical Activity and Potency of Disclosed HDAC6 Inhibitors of Formula (I)

The compounds disclosed herein, in particular those of Formula (I), were synthesized according to methods disclosed in PCT/US2020/066439, published as WO2021127643A1, which is incorporated herein by reference in its entirety. These compounds were tested for potency against HDAC6 and selectivity against HDAC1 in a biochemical assay. A biochemical assay was adopted using a luminescent HDAC-Glo I/II assay (Promega) and measured the relative activity of HDAC6 and HDAC1 recombinant proteins. Compounds were first incubated in the presence of HDAC6 or HDAC1 separately, followed by addition of the luminescent substrate. The data was acquired using a plate reader and the biochemical IC₅₀ were calculated from the data accordingly. Data is tabulated in Table 3. From these studies, it was determined that the compounds of the present disclosure are selective inhibitors of HDAC6 over HDAC1, providing selectivity ratios from about 5 to about 30,0000.

TABLE 3
Characterization Data and HDAC6 Activity for Compounds of Formula (I).

| | | ¹H NMR | HDAC6 |
|---|---|---|---|
| Cmpd | Structure/Name | MS (m/z) (RT) | IC₅₀ (UM) |
| I-1 | 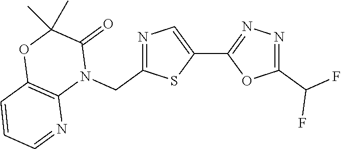 [SMILES: CC1(C)Oc2cccnc2N(Cc2ncc(-c3nnc(C(F)F)o3)s2)C1=O] | ¹H NMR (400 MHz, CDCl₃) δ 8.39 (s, 1 H), 8.01-8.05 (m, 1 H), 7.29 (dd, J = 8.1, 1.5 Hz, 1 H), 7.01 (dd, J = 8.1, 1.5 Hz, 1H), 6.89 (t, J = 51.6 Hz, 1 H), 5.70 (s, 2 H) LCMS: RT = 5.00 min, m/z = 394.0 | 0.107 |
| | 4-((5-(5-(difluoromethyl)-1,3,4- | | |
| | oxadiazol-2-yl)thiazol-2- | | |
| | yl)methyl)-2,2-dimethyl-2H- | | |
| | pyrido[3,2-b][1,4]oxazin-3(4H)- | | |
| | one | | |
| I-2 | 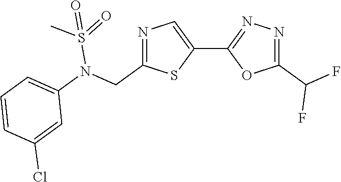 [SMILES: CS(=O)(=O)N(Cc1ncc(-c2nnc(C(F)F)o2)s1)c1cccc(Cl)c1] | ¹H NMR (400 MHz, CDCl₃) δ 8.39 (s, 1 H), 7.48 (s, 1 H), 7.32-7.40 (m, 3 H), 6.91 (t, J = 51.6 Hz, 1 H), 5.25 (s, 2 H), 3.07 (s, 3 H) LCMS: RT = 4.83 min, m/z = 421.0 | 0.021 |
| | N-(3-chlorophenyl)-N-((5-(5- | | |
| | (difluoromethyl)-1,3,4-oxadiazol- | | |
| | 2-yl)thiazol-2- | | |
| | yl)methyl)methanesulfonamide | | |
| I-3 | 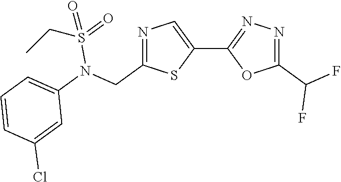 [SMILES: CCS(=O)(=O)N(Cc1ncc(-c2nnc(C(F)F)o2)s1)c1cccc(Cl)c1] | ¹H NMR (400 MHz, CDCl₃) δ 8.37 (s, 1 H), 7.48 (s, 1 H), 7.25-7.40 (m, 3 H), 6.19 (t, J = 51.6 Hz, 1 H), 5.28 (s, 2 H), 3.18 (q, J = 7.6 Hz, 2 H), 1.44 (t, J = 7.2 Hz, 3 H) LCMS: RT = 4.90 min, m/z = 435.0 | 0.0044 |
| | N-(3-chlorophenyl)-N-((5-(5- | | |
| | (difluoromethyl)-1,3,4-oxadiazol- | | |
| | 2-yl)thiazol-2- | | |
| | yl)methyl)ethanesulfonamide | | |
| I-4 | 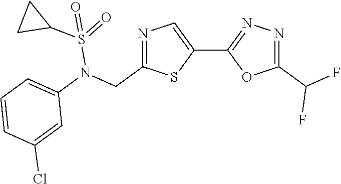 [SMILES: O=S(=O)(C1CC1)N(Cc1ncc(-c2nnc(C(F)F)o2)s1)c1cccc(Cl)c1] | ¹H NMR (400 MHz, DMSO- d₆) δ 8.53 (s, 1 H), 7.40-7.70 (m, 5 H), 5.43 (s, 2 H), 2.95- 3.00 (m, 1 H), 1.00-1.05 (m, 2 H), 0.90-0.95 (m, 2 H) LCMS: RT = 5.10 min, m/z = 447.0 | 0.0042 |
| | N-(3-chlorophenyl)-N-((5-(5- | | |
| | (difluoromethyl)-1,3,4-oxadiazol- | | |
| | 2-yl)thiazol-2- | | |
| | yl)methyl)cyclopropanesulfonamide | | |
| I-5 | 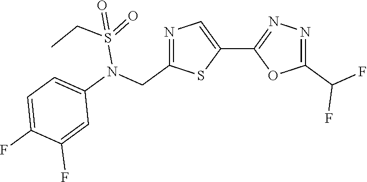 [SMILES: CCS(=O)(=O)N(Cc1ncc(-c2nnc(C(F)F)o2)s1)c1ccc(F)c(F)c1] | ¹H NMR (400 MHz, DMSO- d₆) δ 8.38 (s, 1 H), 7.30-7.40 (m, 1 H), 7.15-7.25 (m, 2 H), 6.91 (t, J = 51.2 Hz, 1 H), 5.24 (s, 2 H), 3.17 (q, J = 7.6 Hz, 2 H), 1.44 (t, J = 5.7 Hz, 3 H) LCMS: RT = 4.92 min, m/z = 437.0 | 0.016 |
| | N-((5-(5-(difluoromethyl)-1,3,4- | | |
| | oxadiazol-2-yl)thiazol-2- | | |
| | yl)methyl)-N-(3,4- | | |
| | difluorophenyl)ethanesulfonamide | | |
| I-6 | 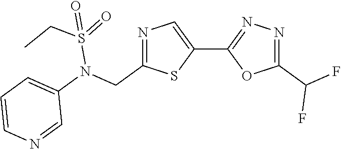 [SMILES: CCS(=O)(=O)N(Cc1ncc(-c2nnc(C(F)F)o2)s1)c1cccnc1] | ¹H NMR (400 MHz, CDCl₃) δ 8.71 (br s, 1 H), 8.58 (br s, 1 H), 8.38 (s, 1 H), 7.80-7.85 (m, 1 H), 7.31-7.35 (m 1 H), 6.91 (t, J = 51.6 Hz, 1 H), 5.30 (s, 2 H), 3.21 (q, J = 7.2 Hz, 2 H), 1.46 (t, J = 7.6 Hz, 1 H) LCMS: RT = 3.39 min, m/z = 402.0 | 0.029 |
| | N-((5-(5-(difluoromethyl)-1,3,4- | | |
| | oxadiazol-2-yl)thiazol-2- | | |
| | yl)methyl)-N-(pyridin-3- | | |
| | yl)ethanesulfonamide | | |
| I-7 | 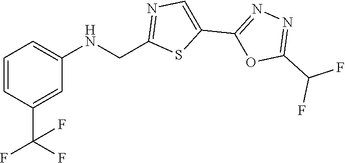 [SMILES: FC(F)c1nnc(-c2cnc(CNc3cccc(C(F)(F)F)c3)s2)o1] | ¹H NMR (400 MHz, CDCl₃) δ 8.58 (s, 1 H) 7.52 (t, J = 51.2 Hz, 1 H) 7.22-7.35 (m, 2 H) 6.89-6.87 (m, 3 H) 4.80 (d, J = 6.1 Hz, 2 H) LCMS: RT 5.20 min, m/z = 377.0 | 0.222 |
| | N-((5-(5-(difluoromethyl)-1,3,4- | | |
| | oxadiazol-2-yl)thiazol-2- | | |
| | yl)methyl)-3- | | |
| | (trifluoromethyl)aniline | | |
| I-8 | 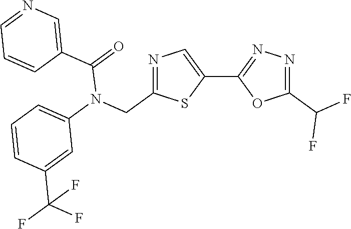 [SMILES: O=C(c1cccnc1)N(Cc1ncc(-c2nnc(C(F)F)o2)s1)c1cccc(C(F)(F)F)c1] | ¹H NMR (400 MHz, CDCl₃) δ 8.50-8.60 (m, 1 H) 8.41 (s, 1 H) 7.71 (d, J = 7.8 Hz, 1 H), 7.50 (d, J = 7.8 Hz, 1 H), 7.35- 7.45 (m, 2 H), 7.25-7.30 (m, 1 H) 7.20-7.25 (m, 1 H) 6.91 (t, J = 51.6 Hz, 1 H) 5.46 (s, 2 H) LCMS: RT = 4.22 min, m/z = 482.0 | 0.088 |
| | N-((5-(5-(difluoromethyl)-1,3,4- | | |
| | oxadiazol-2-yl)thiazol-2- | | |
| | yl)methyl)-N-(3- | | |
| | (trifluoromethyl)phenyl)nicotinamide | | |
| I-9 | 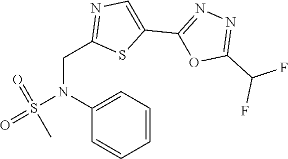 [SMILES: CS(=O)(=O)N(Cc1ncc(-c2nnc(C(F)F)o2)s1)c1ccccc1] | ¹H NMR (400 MHz, CDCl₃) δ = 8.37 (s, 1 H), 7.33-7.49 (m, 5 H), 6.90 (t, 1 H, J = 51.6 Hz) 5.27 (s, 2 H), 3.05 (s, 3 H) ppm LCMS: RT = 4.42 min, m/z = 387.0 | 0.051 |
| | N-((5-(5-(difluoromethyl)-1,3,4- | | |
| | oxadiazol-2-yl)thiazol-2- | | |
| | yl)methyl)-N- | | |
| | phenylmethanesulfonamide | | |
| I-10 | 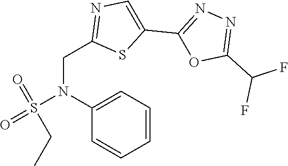 [SMILES: CCS(=O)(=O)N(Cc1ncc(-c2nnc(C(F)F)o2)s1)c1ccccc1] | ¹H NMR (400 MHz, CDCl₃) δ 8.35 (s, 1 H), 7.29-7.53 (m, 5 H), 6.90 (t, 1 H, J = 51.5 Hz), 5.30 (s, 2 H), 3.17 (q, J = 7.34 Hz, 2 H), 1.45 (t, J = 7.46 Hz, 4 H) ppm LCMS: RT = 4.65 min, m/z 401.0 | 0.026 |
| | N-((5-(5-(difluoromethyl)-1,3,4- | | |
| | oxadiazol-2-yl)thiazol-2- | | |
| | yl)methyl)-N- | | |
| | phenylethanesulfonamide | | |
| I-12 | 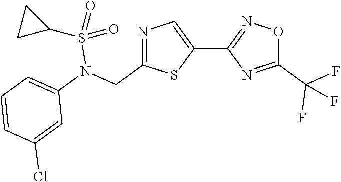 [SMILES: O=S(=O)(C1CC1)N(Cc1ncc(-c2noc(C(F)(F)F)n2)s1)c1cccc(Cl)c1] | ¹H NMR (400 MHz, chloroform-d) δ ppm 8.39 (s, 1 H) 7.51 (s, 1 H) 7.41 (br s, 1 H) 7.32 (br d, J = 3.91 Hz, 2 H) 5.27 (s, 2 H) 2.48-2.59 (m, 1H) 1.10-1.17 (m, 2 H) 0.99- 1.09 (m, 2 H) LCMS: RT = 5.84 min, m/z = 465 | 0.869 |
| | N-(3-chlorophenyl)-N-((5-(5- | | |
| | (trifluoromethyl)-1,2,4-oxadiazol- | | |
| | 3-yl)thiazol-2- | | |
| | yl)methyl)cyclopropanesulfonamide | | |
| I-13 | 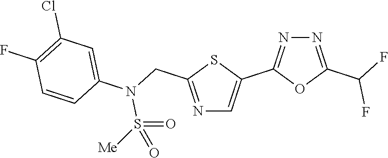 [SMILES: CS(=O)(=O)N(Cc1ncc(-c2nnc(C(F)F)o2)s1)c1ccc(F)c(Cl)c1] | ¹H NMR (400 MHz, CDCl₃) δ 8.40 (s, 1 H) 7.56 (dd, J = 6.36, 2.69 Hz, 1 H) 7.32-7.41 (m, 1 H) 7.18 (t, J = 8.56 Hz, 1 H) 6.76-7.08 (m, 1 H) 5.22 (s, 2 H) 3.07 (s, 3 H) LCMS: RT = 4.93 min, m/z = 438.9 | 0.021 |
| | N-(3-chloro-4-fluorophenyl)-N- | | |
| | ((5-(5-(difluoromethyl)-1,3,4- | | |
| | oxadiazol-2-yl)thiazol-2- | | |
| | yl)methyl)methanesulfonamide | | |
| I-14 | 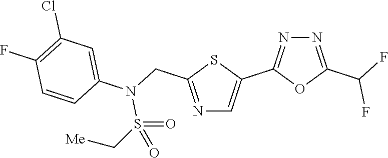 [SMILES: CCS(=O)(=O)N(Cc1ncc(-c2nnc(C(F)F)o2)s1)c1ccc(F)c(Cl)c1] | ¹H NMR (400 MHz, CDCl₃) δ 8.38 (s, 1 H) 7.54 (dd, J = 6.24, 2.32 Hz, 1 H) 7.33-7.40 (m, 1 H) 7.16 (t, J = 8.68 Hz, 1 H) 6.76-7.06 (m, 1 H) 5.23 (s, 2 H) 3.18 (q, J = 7.34 Hz, 2 H) 1.45 (t, J = 7.34 Hz, 3 H) LCMS: RT = 5.11 min, m/z = 453 | 0.014 |
| | N-(3-chloro-4-fluorophenyl)-N- | | |
| | ((5-(5-(difluoromethyl)-1,3,4- | | |
| | oxadiazol-2-yl)thiazol-2- | | |
| | yl)methyl)ethanesulfonamide | | |
| I-15 | 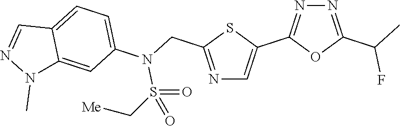 [SMILES: CCS(=O)(=O)N(Cc1ncc(-c2nnc(C(C)F)o2)s1)c1ccc2cnn(C)c2c1] | ¹H NMR (400 MHz, CDCl₃) δ 8.36 (s, 1 H) 7.98 (d, J = 0.98 Hz, 1 H) 7.75 (d, J = 8.56 Hz, 1 H) 7.60 (s, 1 H) 7.18 (d, J = 8.31 Hz, 1H) 6.75-7.05 (m, 1 H) 5.39 (s, 2 H) 4.09 (s, 3 H) 3.19 (q, J = 7.17 Hz, 2 H) 1.46 (t, J = 7.46 Hz, 3 H) | 0.014 |
| | | LCMS: RT = 4.43 min, m/z = | |
| | N-((5-(5-(difluoromethyl)-1,3,4- | 455.0. | |
| | oxadiazol-2-yl)thiazol-2- | | |
| | yl)methyl)-N-(1-methyl-1H- | | |
| | indazol-6-yl)ethanesulfonamide | | |
| I-16 | 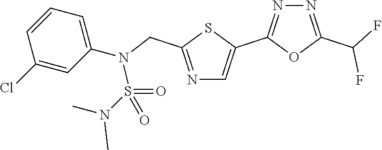 [SMILES: CN(C)S(=O)(=O)N(Cc1ncc(-c2nnc(C(F)F)o2)s1)c1cccc(Cl)c1] | ¹H NMR (400 MHz, CDCl₃) δ 8.36 (s, 1 H) 7.49 (s, 1 H) 7.28-7.42 (m, 3 H) 6.76-6.06 (m, 1 H) 5.17 (s, 2 H) 2.82 (s, 6 H) LCMS: RT = 5.18 min, m/z = 450.0 | 0.012 |
| | [(3-chlorophenyl)({5-[5- | | |
| | (difluoromethyl)-1,3,4-oxadiazol- | | |
| | 2-yl]-1,3-thiazol-2- | | |
| | yl}methyl)sulfamoyl]dimethylamine | | |
| I-17 | 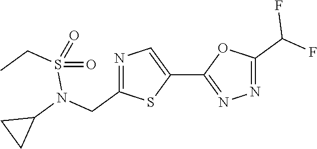 [SMILES: CCS(=O)(=O)N(Cc1ncc(-c2nnc(C(F)F)o2)s1)C1CC1] | ¹H NMR (400 MHz, chloroform-d) δ ppm 8.42 (s, 1 H) 6.74-7.07 (m, 1 H) 4.83 (s, 2 H) 3.21 (q, J = 7.50 Hz, 2 H) 2.70 (br s, 1 H) 1.41 (t, J = 7.46 Hz, 3 H) 0.83-0.95 (m, 4 H) LCMS: RT = 4.34 min, m/z = 365.0 | 1.23 |
| | N-cyclopropyl-N-((5-(5- | | |
| | (difluoromethyl)-1,3,4-oxadiazol- | | |
| | 2-yl)thiazol-2- | | |
| | yl)methyl)ethanesulfonamide | | |
| I-18 | 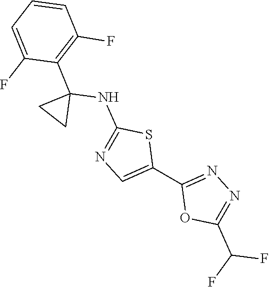 [SMILES: Fc1cccc(F)c1C1(Nc2ncc(-c3nnc(C(F)F)o3)s2)CC1] | ¹H NMR (400 MHz, DMSO- d₆) δ ppm 9.62 (br s, 1 H) 7.95 (br s, 1 H) 7.29-7.67 (overlapping m, 2 H) 7.02- 7.18 (m, 2 H) 1.35 (d, J = 26.80 Hz, 4 H) LCMS: RT = 4.87 min, m/z = 371.1 | 1.95 |
| | 5-(5-(difluoromethyl)-1,3,4- | | |
| | oxadiazol-2-yl)-N-(1-(2,6- | | |
| | difluorophenyl)cyclopropyl)thiazo | | |
| | 1-2-amine | | |
| I-19 | 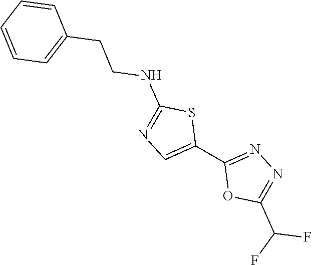 [SMILES: FC(F)c1nnc(-c2cnc(NCCc3ccccc3)s2)o1] | ¹H NMR (400 MHz, chloroform-d) δ ppm 7.86 (s, 1 H) 7.16-7.46 (m, 5 H) 6.68- 7.03 (m, 1 H) 5.75 (br s, 1 H) 3.64 (br d, J = 6.11 Hz, 2 H) 3.02 (br t, J = 6.85 Hz, 2 H) LCMS: RT = 4.73 min, m/z = 323.0. | 13.6 |
| | 5-(5-(difluoromethyl)-1,3,4- | | |
| | oxadiazol-2-yl)-N- | | |
| | phenethylthiazol-2-amine | | |
| I-20 | 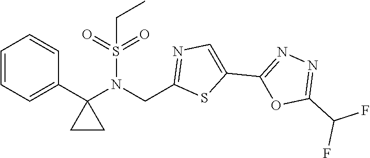 [SMILES: CCS(=O)(=O)N(Cc1ncc(-c2nnc(C(F)F)o2)s1)C1(c2ccccc2)CC1] | ¹H NMR (400 MHz, CDCl₃-d₃) δ 8.78 (s, 1 H), 8.40 (s, 1 H), 7.50 (d, J = 7.34 Hz, 2 H), 7.26- 7.38 (m, 4 H), 7.04 (s, 0.25 H), 6.91 (s, 0.5 H), 6.78 (s, 0.25 H), 4.94 (s, 2 H), 2.81 (q, J = 7.58 Hz, 2 H), 1.47 (m, 2 H), 1.31-1.22 (m, 5 H). LCMS: RT = 6.12, m/z = 441.1 | 4.14 |
| | N-((5-(5-(difluoromethyl)-1,3,4- | | |
| | oxadiazol-2-yl)thiazol-2- | | |
| | yl)methyl)-N-(1- | | |
| | phenylcyclopropyl)ethanesulfonamide | | |
| I-21 | 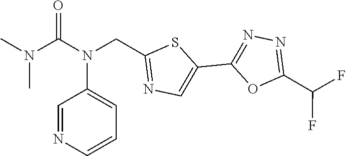 [SMILES: CN(C)C(=O)N(Cc1ncc(-c2nnc(C(F)F)o2)s1)c1cccnc1] | ¹H NMR (400 MHz, METHANOL-d4) δ ppm 9.55 (s, 1 H) 8.78 (br d, J = 5.62 Hz, 1 H) 8.52-8.59 (m, 2 H) 8.06 (br d, J = 7.83 Hz, 1 H) 7.26 (t, J = 51.2 Hz, 1 H) 6.33 (s, 2 H) 3.09 (s, 6 H). LCMS RT = 2.85 min, m/z = 381.1 | 1.2 |
| | 1-((5-(5-(difluoromethyl)-1,3,4- | | |
| | oxadiazol-2-yl)thiazol-2- | | |
| | yl)methyl)-3,3-dimethyl-1- | | |
| | (pyridin-3-yl)urea | | |
| I-22 | 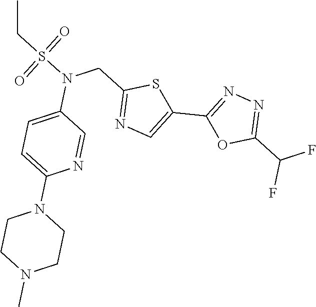 [SMILES: CCS(=O)(=O)N(Cc1ncc(-c2nnc(C(F)F)o2)s1)c1ccc(N2CCN(C)CC2)nc1] | ¹H NMR (400 MHz, CD3OD) δ 8.40 (s, 1 H), 8.15 (s, 1 H), 7.63 (m, 1 H), 7.21 (t, J = 51.2 Hz, 1H), 6.79 (m, 1 H), 5.24 (s, 2 H), 3.56 (m, 4 H), 3.25 (q, J = 7.2 Hz, 2 H), 2.51 (m, 4 H), 2.32 (s, 3 H), 1.40 (t, J = 7.2 Hz, 3 H). m/z = 500.1 | 0.143 |
| | N-((5-(5-(difluoromethyl)-1,3,4- | | |
| | oxadiazol-2-yl)thiazol-2- | | |
| | yl)methyl)-N-(6-(4- | | |
| | methylpiperazin-1-yl)pyridin-3- | | |
| | yl)ethanesulfonamide | | |
| I-23 | 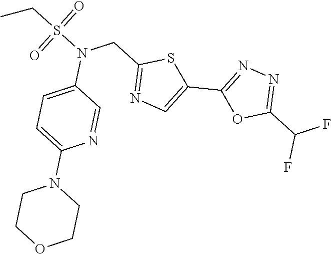 [SMILES: CCS(=O)(=O)N(Cc1ncc(-c2nnc(C(F)F)o2)s1)c1ccc(N2CCOCC2)nc1] | ¹H NMR (400 MHz, DMSO- d6) δ 8.51 (s, 1 H), 8.18 (s, 1 H), 7.69 (m, 1 H), 7.54 (t, J = 51.2 Hz, 1 H), 6.83 (m, 1 H), 5.28 (s, 1 H), 3.66 (m, 4 H), 3.44 (m, 4 H), 3.30 (q, J = 6.8 Hz, 2 H), 1.29 (t, J = 6.8 Hz, 3 H) m/z = 487.1 | 0.417 |
| | N-((5-(5-(difluoromethyl)-1,3,4- | | |
| | oxadiazol-2-yl)thiazol-2- | | |
| | yl)methyl)-N-(6- | | |
| | morpholinopyridin-3- | | |
| | yl)ethanesulfonamide | | |
| I-24 | 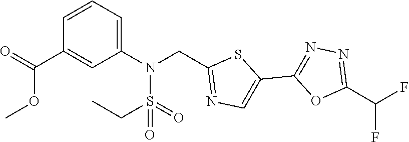 [SMILES: CCS(=O)(=O)N(Cc1ncc(-c2nnc(C(F)F)o2)s1)c1cccc(C(=O)OC)c1] | ¹H NMR (400 MHz, DMSO- d₆) δ 8.50 (s, 1 H), 8.08 (s, 1 H), 7.95-7.83 (m, 2 H), 7.67- 7.39 (m, 2 H), 5.43 (s, 2 H), 3.86 (s, 3 H), 3.45-3.33 (m, 2 ), 1.28 (t, J = 7.21 Hz, 3 H). LCMS RT = 4.73 min, m/z = 459.1 | 0.031 |
| | methyl 3-(N-((5-(5- | | |
| | (difluoromethyl)-1,3,4-oxadiazol- | | |
| | 2-yl)thiazol-2- | | |
| | yl)methyl)ethylsulfonamido)benzoate | | |
| I-25 | 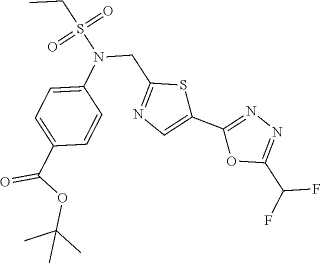 [SMILES: CCS(=O)(=O)N(Cc1ncc(-c2nnc(C(F)F)o2)s1)c1ccc(C(=O)OC(C)(C)C)cc1] | ¹H NMR (400 MHz, CDCl₃) δ 8.36 (s, 1 H), 8.01 (m, 2 H), 7.53 (m, 2 H), 6.91 (t, J = 51.6 Hz, 1 H), 5.34 (s, 2 H), 3.21 (q, J = 7.2 Hz, 2 H), 1,57 (s, 9 H), 1.43 (t, J = 7.2 Hz, 3 H). m/z = 501.1 | 0.101 |
| | tert-butyl 4-(N-((5-(5- | | |
| | (difluoromethyl)-1,3,4-oxadiazol- | | |
| | 2-yl)thiazol-2- | | |
| | yl)methyl)ethylsulfonamido)benzoate | | |
| I-26 | 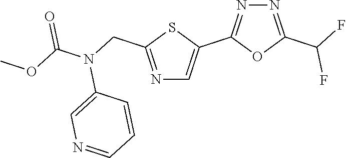 [SMILES: COC(=O)N(Cc1ncc(-c2nnc(C(F)F)o2)s1)c1cccnc1] | ¹H NMR (400 MHz, METHANOL-d4) δ ppm 9.55 (s, 1 H) 8.85 (br d, J = 5.87 Hz, 1 H) 8.57 (s, 1 H) 8.45 (br d, J = 8.80 Hz, 1 H) 8.06-8.13 (m, 1 H) 7.26 (t, J = 51.2 Hz, 1 H) 3.86 (s, 3 H). LCMS RT = 2.97 min, m/z = 368.0 | 2.5 |
| | methyl ((5-(5-(difluoromethyl)- | | |
| | 1,3,4-oxadiazol-2-yl)thiazol-2- | | |
| | yl)methyl)(pyridin-3-yl)carbamate | | |
| I-27 | 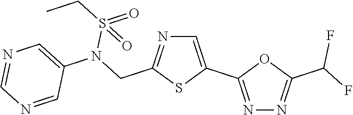 [SMILES: CCS(=O)(=O)N(Cc1ncc(-c2nnc(C(F)F)o2)s1)c1cncnc1] | ¹H NMR (400 MHz, DMSO- d₆) δ 9.12 (s, 1 H), 9.00 (s, 2 H), 8.53 (s, 1 H), 7.70-7.30 (m, 1 H), 5.50 (s, 2 H), 3.55-3.40 (m, 2 H), 1.31 (t, −7.34 Hz, 3 H). LCMS RT = 3.84 min, m/z = 403.0 | 0.050 |
| | N-((5-(5-(difluoromethyl)-1,3,4- | | |
| | oxadiazol-2-yl)thiazol-2- | | |
| | yl)methyl)-N-(pyrimidin-5- | | |
| | yl)ethanesulfonamide | | |
| I-28 | 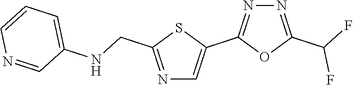 [SMILES: FC(F)c1nnc(-c2cnc(CNc3cccnc3)s2)o1] | ¹H NMR (400 MHz, METHANOL-d4) δ ppm 8.58 (s, 1 H) 8.22-8.32 (m, 2 H) 7.69-7.78 (m, 2 H) 7.26 (t, J = 51.2 Hz, 1 H) 6.17 (s, 2 H). LCMS RT = 1.24 min, m/z = 310.1 | 1.5 |
| | N-((5-(5-(difluoromethyl)-1,3,4- | | |
| | oxadiazol-2-yl)thiazol-2- | | |
| | yl)methyl)pyridin-3-amine | | |
| I-29 | 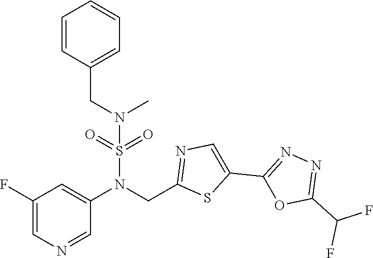 [SMILES: CN(Cc1ccccc1)S(=O)(=O)N(Cc1ncc(-c2nnc(C(F)F)o2)s1)c1cncc(F)c1] | ¹H NMR (400 MHz, CDCl₃-d₃) δ 8.55 (s, 1 H), 8.47 (s, 1 H), 8.39 (s, 1 H), 7.67 (d, J = 9.05 Hz, 1 H), 7.36-7.28 (m, 2 H), 7.19 (d, J = 6.60 Hz, 2 H), 7.04- 6.77 (m, 1 H), 5.22 (s, 2 H), 4.25 (s, 2 H), 2.74 (s, 3 H). LCMS RT = 5.23 min, m/z = 511.1 | 0.041 |
| I-30 | 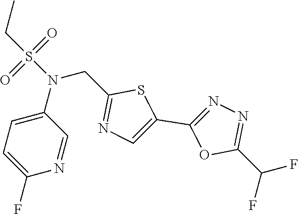 [SMILES: CCS(=O)(=O)N(Cc1ncc(-c2nnc(C(F)F)o2)s1)c1ccc(F)nc1] | ¹H NMR (400 MHz, CDCl₃) δ 8.39 (s, 1 H), 8.31 (s, 1 H), 7.94 (m, 1 H), 7.02 (m, 1 H), 6.91 (t, J = 51.6 Hz, 1 H), 5.25 (s, 2 H), 3.21 (q, J = 7.2 Hz, 2 H), 1.21 (t, J = 7.2 Hz, 3 H). m/z = 420.0 | 0.011 |
| | N-((5-(5-(difluoromethyl)-1,3,4- | | |
| | oxadiazol-2-yl)thiazol-2- | | |
| | yl)methyl)-N-(6-fluoropyridin-3- | | |
| | yl)ethanesulfonamide | | |
| I-31 | 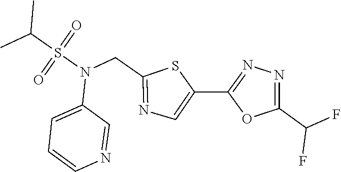 [SMILES: CC(C)S(=O)(=O)N(Cc1ncc(-c2nnc(C(F)F)o2)s1)c1cccnc1] | ¹H NMR (400 MHz, DMSO- d6) δ 8.72 (s, 1 H), 8.50 (m, 2 H), 8.02 (m, 1 H), 7.40-7.66 (m, 2 H), 5.46 (s, 2 H), 3.60 (m, 1 H), 1.34 (d, J = 6.8 Hz, 6 H). m/z = 416.1 | 0.042 |
| | N-((5-(5-(difluoromethyl)-1,3,4- | | |
| | oxadiazol-2-yl)thiazol-2- | | |
| | yl)methyl)-N-(pyridin-3- | | |
| | yl)propane-2-sulfonamide | | |
| I-32 | 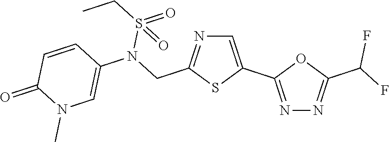 [SMILES: CCS(=O)(=O)N(Cc1ncc(-c2nnc(C(F)F)o2)s1)c1ccc(=O)n(C)c1] | ¹H NMR (400 MHz, Methanol- d₄) δ 8.47 (s, 1 H), 7.96 (s, 1 H), 7.66 (d, J = 9.54, 1 H), 7.35 (s, 0.25 H), 7.22 (s, 0.5 H), 7.09 (s, 0.25 H), 6.53 (d, J = 9.54 Hz, 1 H), 5.22 (s, 2 H), 3.56 (s, 3 H), 3.35-3.30 (m, 2 H), 1.43 (t, J = 7.34 Hz, 3 H). LCMS RT = 3.53 min, m/z = 432.1 | 0.411 |
| | N-((5-(5-(difluoromethyl)-1,3,4- | | |
| | oxadiazol-2-yl)thiazol-2- | | |
| | yl)methyl)-N-(1-methyl-6-oxo- | | |
| | 1,6-dihydropyridin-3- | | |
| | yl)ethanesulfonamide | | |
| I-33 | 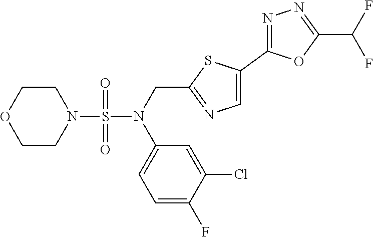 [SMILES: O=S(=O)(N1CCOCC1)N(Cc1ncc(-c2nnc(C(F)F)o2)s1)c1ccc(F)c(Cl)c1] | ¹H NMR (400 MHz, DMSO- d6) δ 8.52 (s, 1 H), 7.86-7.95 (m, 1 H), 7.35-7.69 (m, 3 H), 5.36 (s, 2 H), 3.58 (m, 4 H), 3.20 (m, 4 H). m/z = 510.0 | 0.047 |
| | N-(3-chloro-4-fluorophenyl)-N- | | |
| | ((5-(5-(difluoromethyl)-1,3,4- | | |
| | oxadiazol-2-yl)thiazol-2- | | |
| | yl)methyl)morpholine-4- | | |
| | sulfonamide | | |
| I-34 | 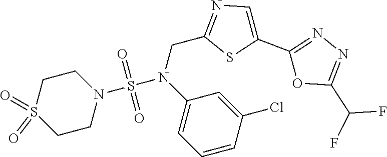 [SMILES: O=S1(=O)CCN(S(=O)(=O)N(Cc2ncc(-c3nnc(C(F)F)o3)s2)c2cccc(Cl)c2)CC1] | ¹H NMR (400 MHz, CHLOROFORM-d) δ ppm 8.41 (s, 1 H) 7.48 (s, 1 H) 7.30- 7.40 (m, 3 H) 6.91 (t, J = 52.4 Hz, 1 H) 5.15 (s, 2 H) 3.73- 3.82 (m, 4H) 3.05-3.13 (m, 4 H). LCMS RT = 4.83 min, m/z = 540.0 | 0.022 |
| | N-(3-chlorophenyl)-N-((5-(5- | | |
| | (difluoromethyl)-1,3,4-oxadiazol- | | |
| | 2-yl)thiazol-2- | | |
| | yl)methyl)thiomorpholine-4- | | |
| | sulfonamide 1,1-dioxide | | |
| I-35 | 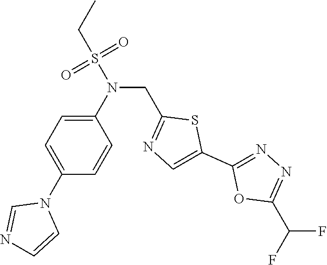 [SMILES: CCS(=O)(=O)N(Cc1ncc(-c2nnc(C(F)F)o2)s1)c1ccc(-n2ccnc2)cc1] | ¹H NMR (400 MHz, DMSO- d6) δ 8.51 (s, 1 H), 8.27 (s, 1 H), 7.69-7.76 (m, 5 H), 7.3 (t, J = 51.2 Hz, 1 H), 7.10 (s, 1 H), 5.41 (s, 2 H), 3.37 (q, J = 6.8 Hz, 2 H), 1.30 (t, J = 6.8 Hz, 3 H). m/z = 467.0 | 0.008 |
| | N-(4-(1H-imidazol-1-yl)phenyl)- | | |
| | N-((5-(5-(difluoromethyl)-1,3,4- | | |
| | oxadiazol-2-yl)thiazol-2- | | |
| | yl)methyl)ethanesulfonamide | | |
| I-36 | 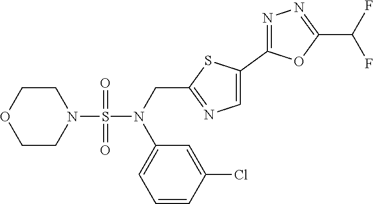 [SMILES: O=S(=O)(N1CCOCC1)N(Cc1ncc(-c2nnc(C(F)F)o2)s1)c1cccc(Cl)c1] | ¹H NMR (400 MHz, DMSO- d6) δ 8.52 (s, 1 H), 7.73 (m, 1 H), 7.54-7.58 (m, 2 H), 7.36- 7.44 (m, 2 H), 5.38 (s, 2 H), 3.59 (m, 4 H), 3.18 (m, 4 H). m/z = 492.1 | 0.005 |
| | N-(3-chlorophenyl)-N-((5-(5- | | |
| | (difluoromethyl)-1,3,4-oxadiazol- | | |
| | 2-yl)thiazol-2- | | |
| | yl)methyl)morpholine-4- | | |
| | sulfonamide | | |
| I-37 | 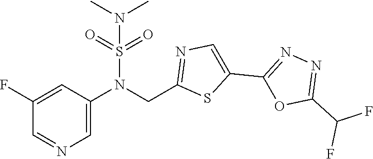 [SMILES: CN(C)S(=O)(=O)N(Cc1ncc(-c2nnc(C(F)F)o2)s1)c1cncc(F)c1] | ¹H NMR (400 MHz, DMSO- d₆) δ 8.63 (s, 1 H), 8.54 (d, J =9. Hz, 2 H), 8.10 (d, J =10.0 Hz, 1 H), 7.65-7.39 (m, 1 H), 5.41 (s, 2 H), 2.83 (s, 6 H). LCMS RT = 4.39 min, m/z = 435.1 | 0.013 |
| I-38 | 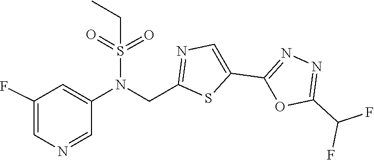 [SMILES: CCS(=O)(=O)N(Cc1ncc(-c2nnc(C(F)F)o2)s1)c1cncc(F)c1] | ¹H NMR (400 MHz, DMSO- d₆) δ 8.62-8.52 (m, H), 8.13- 8.09 (m, 1 H), 7.65-7.39 (m, 1 H), 5.46 (s, 2 H), 3.55-3.45 (m, 2 H), 1.30 (t, J = 7.34 Hz, 3 H). LCMS RT = 4.32 min, m/z = 420.0 | 0.019 |
| | N-((5-(5-(difluoromethyl)-1,3,4- | | |
| | oxadiazol-2-yl)thiazol-2- | | |
| | yl)methyl)-N-(5-fluoropyridin-3- | | |
| | yl)ethanesulfonamide | | |
| I-39 | 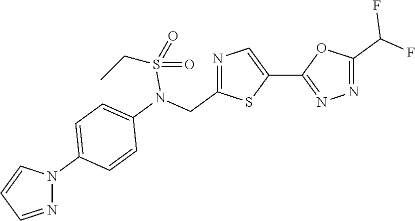 [SMILES: CCS(=O)(=O)N(Cc1ncc(-c2nnc(C(F)F)o2)s1)c1ccc(-n2cccn2)cc1] | ¹H NMR (400 MHz, CHLOROFORM-d) δ ppm 8.36 (s, 1 H) 7.90 (s, 1 H) 7.69- 7.77 (m, 3 H) 7.55 (d, J = 8.31 Hz, 2 H) 6.90 (t, J = 52.0 Hz, 1 H) 6.48 (s, 1 H) 5.31 (s, 2 H) 3.19 (d, J = 7.34 Hz, 2 H) 1.46 (t, J = 7.46 Hz, 3 H). LCMS RT = 4.67 min, m/z = 467.0 | 0.010 |
| | N-(4-(1H-pyrazol-1-yl)phenyl)-N- | | |
| | ((5-(5-(difluoromethyl)-1,3,4- | | |
| | oxadiazol-2-yl)thiazol-2- | | |
| | yl)methyl)ethanesulfonamide | | |
| I-40 | 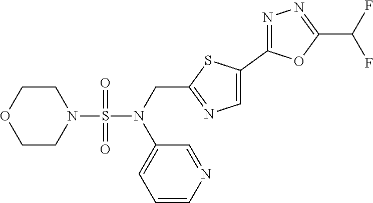 [SMILES: O=S(=O)(N1CCOCC1)N(Cc1ncc(-c2nnc(C(F)F)o2)s1)c1cccnc1] | ¹H NMR (400 MHz, CD3OD) δ 8.57 (s, 1 H), 8.45 (s, 1 H), 8.27 (m, 1 H), 7.97 (m, 1 H), 7.73 (m, 1 H), 7.22 (t, J = 51.6 Hz, 1 H), 4.88 (s, 2 H), 3.36 (m, 4 H), 3.10 (m, 4 H). m/z = 459.1 | 4.22 |
| | N-((5-(5-(difluoromethyl)-1,3,4- | | |
| | oxadiazol-2-yl)thiazol-2- | | |
| | yl)methyl)-N-(pyridin-3- | | |
| | yl)morpholine-4-sulfonamide | | |
| I-43 | 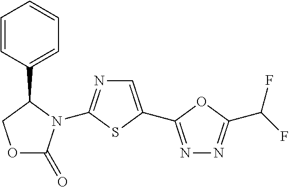 [SMILES: O=C1OC[C@@H](c2ccccc2)N1c1ncc(-c2nnc(C(F)F)o2)s1] | ¹H NMR (400 MHz, METHANOL- d4) δ = 8.07 (s, 1 H) 7.40 (s, 5 H) 7.19 (t, 1 H, J = 51.6 Hz) 5.84 (dd, J = 8.80, 3.67 Hz, 1 H) 5.03 (t, J = 8.80 Hz, 1 H) 4.48 (dd, J = 8.93, 4.03 Hz, 1 H) ppm LCMS: RT = 4.67 min, m/z = 365.1 | 0.085 |
| | (R)-3-(5-(5-(difluoromethyl)- | | |
| | 1,3,4-oxadiazol-2-yl)thiazol-2-yl)- | | |
| | 4-phenyloxazolidin-2-one | | |
| I-44 | 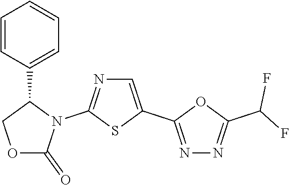 [SMILES: O=C1OC[C@H](c2ccccc2)N1c1ncc(-c2nnc(C(F)F)o2)s1] | 1H NMR (400 MHz, METHANOL- d4) δ = 8.07 (s, 1 H), 7.40 (s, 4 H), 7.33-7.38 (m, 1 H), 7.19 (t, 1 H, J = 5.6 Hz), 5.84 (dd, J = 8.56, 3.91 Hz, 1 H), 5.03 (t, J = 8.93 Hz, 1 H), 4.43-4.51 (m, 1 H) ppm LCMS: RT = 4.95 min, m/z = 365.1 | 8.97 |
| | (S)-3-(5-(5-(difluoromethyl)-1,3,4- | | |
| | oxadiazol-2-yl)thiazol-2-yl)-4- | | |
| | phenyloxazolidin-2-one | | |
| I-45 | 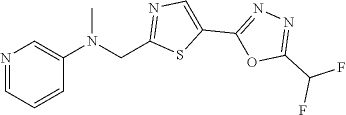 [SMILES: CN(Cc1ncc(-c2nnc(C(F)F)o2)s1)c1cccnc1] | 1H NMR (400 MHz, CD3OD) δ ppm 8.55 (s, 1 H), 8.27- 8.24 (m, 2 H), 7.78-7.67 (m, 2 H), 7.23 (t, J = 51.6 Hz, 1 H), 6.15 (s, 2 H), 2.90 (s, 3 H). LCMS RT = 2.431 min, m/z = 324.0 | 1.3 |
| | N-(1-(cyclopropylmethyl)-1H- | | |
| | pyrazol-4-yl)-N-((5-(5- | | |
| | (difluoromethyl)-1,3,4-oxadiazol- | | |
| | 2-yl)thiazol-2- | | |
| | yl)methyl)ethanesulfonamide | | |
| I-46 | 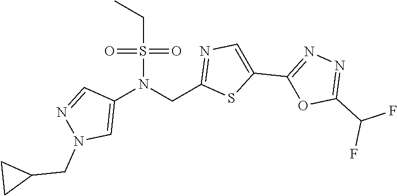 [SMILES: CCS(=O)(=O)N(Cc1ncc(-c2nnc(C(F)F)o2)s1)c1cnn(CC2CC2)c1] | 1H NMR (400 MHz, CDCl3) δ ppm 8.40 (s, 1 H), 7.69 (s, 1 H), 7.49 (s, 1 H), 6.91 (t, J = 51.6 Hz, 1 H), 5.17 (s, 2 H), 3.93 (d, J = 7.2 Hz, 2 H), 3.18 (q, J = 7.6 Hz, 2 H), 1.42 (t, J = 7.6 Hz, 3 H), 1.31-1.22 (m, 1 H), 0.70-0.62 (m, 2 H), 0.38- 0.32 (m, 2 H). LCMS RT = 2.417 min, m/z = 444.9 | 0.077 |
| | N-(1-(cyclopropylmethyl)-1H- | | |
| | pyrazol-4-yl)-N-((5-(5- | | |
| | (difluoromethyl)-1,3,4-oxadiazol- | | |
| | 2-yl)thiazol-2- | | |
| | yl)methyl)ethanesulfonamide | | |
| I-47 | 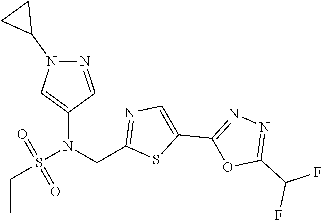 [SMILES: CCS(=O)(=O)N(Cc1ncc(-c2nnc(C(F)F)o2)s1)c1cnn(C2CC2)c1] | ¹H NMR (400 MHz, CDCl3) δ ppm 8.40 (s, 1 H), 7.65 (s, 1 H), 7.46 (s, 1 H), 6.91 (t, J = 51.6 Hz, 1 H), 5.14 (s, 2 H), 3.58-3.52 (m, 1 H), 3.16 (q, J = 8.0 Hz , 2 H), 1.42 (t, J = 8.0 Hz, 3 H), 1.13-1.00 (m, 4 H). LCMS RT = 1.223 min, m/z = 430.9 | 0.070 |
| | N-(1-cyclopropyl-1H-pyrazol-4- | | |
| | yl)-N-((5-(5-(difluoromethyl)- | | |
| | 1,3,4-oxadiazol-2-yl)thiazol-2- | | |
| | yl)methyl)ethanesulfonamide | | |
| I-48 | 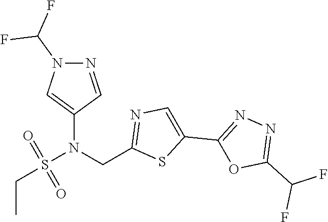 [SMILES: CCS(=O)(=O)N(Cc1ncc(-c2nnc(C(F)F)o2)s1)c1cnn(C(F)F)c1] | 1H NMR (400 MHz, CDCl3) δ ppm 8.35 (s, 1 H), 7.92 (s, 1 H), 7.66 (s, 1 H), 6.96 (t, J = 60.4 Hz, 1 H), 6.84 (t, J = 45.2 Hz, 1 H), 5.11 (s, 2 H), 3.10 (q, J = 7.2 Hz, 2 H), 1.33 (t, J = 7.6 Hz, 3 H). LCMS RT = 3.543 min, m/z = 441.1 | 0.262 |
| | N-((5-(5-(difluoromethyl)-1,3,4- | | |
| | oxadiazol-2-yl)thiazol-2- | | |
| | yl)methyl)-N-(1-(difluoromethyl)- | | |
| | 1H-pyrazol-4- | | |
| | yl)ethanesulfonamide | | |
| I-49 | 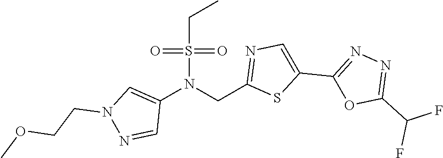 [SMILES: CCS(=O)(=O)N(Cc1ncc(-c2nnc(C(F)F)o2)s1)c1cnn(CCOC)c1] | 1H NMR (400 MHz, CDCl3) δ ppm 8.40 (s, 1 H), 7.65 (s, 1 H), 7.51 (s, 1 H), 6.91 (t, J = 51.6 Hz, 1 H), 5.15 (s, 2 H), 4.22 (t, J = 4.8 Hz, 2 H), 3.70 (t, J = 5.2 Hz, 2 H), 3.30 (s, 3 H), 3.17 (q, J = 7.6 Hz, 2 H), 1.41 (t, J = 7.2 Hz, 3 H). LCMS RT = 3.093 min, m/z = 448.9 | 0.134 |
| | N-((5-(5-(difluoromethyl)-1,3,4- | | |
| | oxadiazol-2-yl)thiazol-2- | | |
| | yl)methyl)-N-(1-(2- | | |
| | methoxyethyl)-1H-pyrazol-4- | | |
| | yl)ethanesulfonamide | | |
| I-50 | 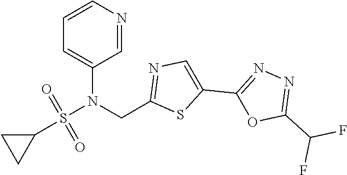 [SMILES: O=S(=O)(C1CC1)N(Cc1ncc(-c2nnc(C(F)F)o2)s1)c1cccnc1] | 1H NMR (400 MHz, CDCl3) δ ppm 8.69 (d, J = 2.0 Hz, 1 H), 8.53-8.50 (m, 1 H), 8.30 (s, 1 H), 7.79-7.76 (m, 1 H), 7.30- 7.26 (m, 1 H), 6.83 (t, J = 51.6 Hz, 1 H), 5.23 (s, 2 H), 2.53-2.46 (m, 1 H), 1.06- 0.95 (m, 4 H). LCMS RT = 2.897 min, m/z = 414.1 | 0.036 |
| | N-((5-(5-(difluoromethyl)-1,3,4- | | |
| | oxadiazol-2-yl)thiazol-2- | | |
| | yl)methyl)-N-(pyridin-3- | | |
| | yl)cyclopropanesulfonamide | | |
| I-51 | 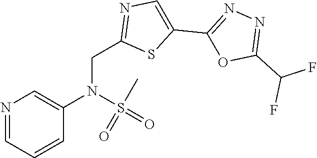 [SMILES: CS(=O)(=O)N(Cc1ncc(-c2nnc(C(F)F)o2)s1)c1cccnc1] | 1H NMR (400 MHz, CDCl3) δ ppm 8.54 (s, 1 H), 8.50 (s, 1 H), 7.93-7.90 (m, 1 H), 7.68 (d, J = 8.0 Hz, 1 H), 7.49- 7.44 (m, 1 H), 6.92 (t, J = 51.6 Hz, 1 H), 5.84 (s, 2 H), 3.00 (s, 3 H). LCMS RT = 1.096 min, m/z = 387.9 | 0.676 |
| | N-((5-(5-(difluoromethyl)-1,3,4- | | |
| | oxadiazol-2-yl)thiazol-2- | | |
| | yl)methyl)-N-(pyridin-3- | | |
| | yl)methanesulfonamide | | |
| I-52 | 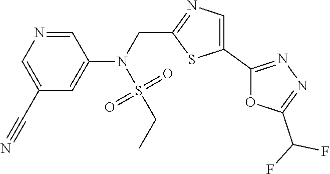 [SMILES: CCS(=O)(=O)N(Cc1ncc(-c2nnc(C(F)F)o2)s1)c1cncc(C#N)c1] | 1H NMR (400 MHz, CDCl3) δ ppm 8.96 (d, J = 2.8 Hz, 1 H), 8.82 (s, 1 H), 8.42 (s, 1 H), 8.18-8.17 (m, 1 H), 6.92 (t, J = 51.6 Hz, 1 H), 5.30 (s, 2 H), 3.21 (q, J = 7.6 Hz, 2 H), 1.44 (t, J = 7.6 Hz, 3 H). LCMS RT = 0.752 min, m/z = 427.1 | 0.022 |
| | N-(5-cyanopyridin-3-yl)-N-((5-(5- | | |
| | (difluoromethyl)-1,3,4-oxadiazol- | | |
| | 2-yl)thiazol-2- | | |
| | yl)methyl)ethanesulfonamide | | |
| I-53 | 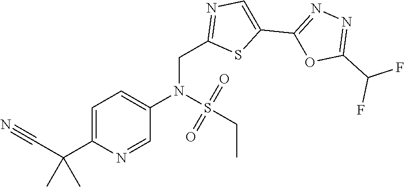 [SMILES: CCS(=O)(=O)N(Cc1ncc(-c2nnc(C(F)F)o2)s1)c1ccc(C(C)(C)C#N)nc1] | 1H NMR (400 MHz, CDCl3) δ ppm 8.70 (d, J = 2.8 Hz, 1 H), 8.40 (s, 1 H), 7.86 (d, J = 8.4 Hz, 1 H), 7.63 (d, J = 8.4 Hz, 1 H), 6.91 (t, J = 51.6 Hz, 1 H), 5.29 (s, 2 H), 3.22 (q, J = 7.6 Hz, 2 H), 1.75 (s, 6 H), 1.47 (t, J = 7.2 Hz, 3 H). LCMS RT = 0.828 min, m/z = 469.2 | 0.317 |
| | N-(6-(2-cyanopropan-2-yl)pyridin- | | |
| | 3-yl)-N-((5-(5-(difluoromethyl)- | | |
| | 1,3,4-oxadiazol-2-yl)thiazol-2- | | |
| | yl)methyl)ethanesulfonamide | | |
| I-54 | 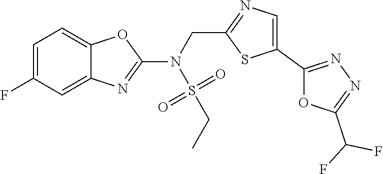 [SMILES: CCS(=O)(=O)N(Cc1ncc(-c2nnc(C(F)F)o2)s1)c1nc2cc(F)ccc2o1] | 1H NMR (400 MHz, CDCl3) δ ppm 8.45 (s, 1 H), 7.40-7.38 (m, 1 H), 7.10-6.91 (m, 3 H), 5.40 (s, 2 H), 3.29 (q, J = 7.2 Hz, 2 H), 1.52 (t, J = 7.2 Hz, 3 H). LCMS RT = 3.800 min, m/z = 460.0 | 0.104 |
| | N-((5-(5-(difluoromethyl)-1,3,4- | | |
| | oxadiazol-2-yl)thiazol-2- | | |
| | yl)methyl)-N-(5- | | |
| | fluorobenzo[d]oxazol-2- | | |
| | yl)ethanesulfonamide | | |
| I-55 | 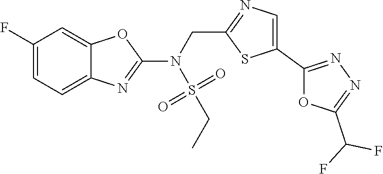 [SMILES: CCS(=O)(=O)N(Cc1ncc(-c2nnc(C(F)F)o2)s1)c1nc2ccc(F)cc2o1] | 1H NMR (400 MHz, CDCl3) δ ppm 8.44 (s, 1 H), 7.29-7.21 (m, 2 H), 7.10-7.04 (m, 1 H), 6.91 (t, J = 51.6 Hz, 1 H), 5.42 (s, 2 H), 3.29 (q, J = 7.2 Hz, 2 H), 1.52 (t, J = 7.2 Hz, 3 H). LCMS RT = 0.863 min, m/z = 459.7 | 0.124 |
| | N-((5-(5-(difluoromethyl)-1,3,4- | | |
| | oxadiazol-2-yl)thiazol-2- | | |
| | yl)methyl)-N-(6- | | |
| | fluorobenzo[d]oxazol-2- | | |
| | yl)ethanesulfonamide | | |
| I-56 | 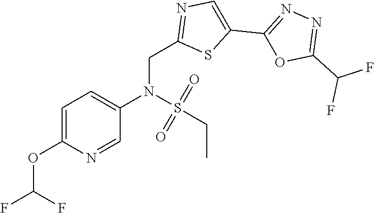 [SMILES: CCS(=O)(=O)N(Cc1ncc(-c2nnc(C(F)F)o2)s1)c1ccc(OC(F)F)nc1] | 1H NMR (400 MHz, CDCl3) δ ppm 8.39 (s, 1 H), 8.28 (d, J = 2.8 Hz, 1 H), 7.85 (d, J = 8.8 Hz, 1 H), 7.41 (t, J = 51.6 Hz, 1 H), 7.06-6.76 (m, 2 H), 5.24 (s, 2 H), 3.20 (q, J = 7.2 Hz, 2 H), 1.47 (t, J = 7.6 Hz, 3 H). LCMS RT = 0.886 min, m/z = 468.1 | 0.050 |
| | N-(6-(difluoromethoxy)pyridin-3- | | |
| | yl)-N-((5-(5-(difluoromethyl)- | | |
| | 1,3,4-oxadiazol-2-yl)thiazol-2- | | |
| | yl)methyl)ethanesulfonamide | | |
| I-57 | 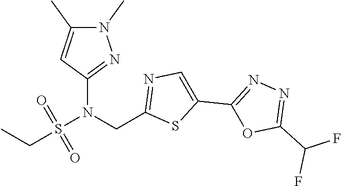 [SMILES: CCS(=O)(=O)N(Cc1ncc(-c2nnc(C(F)F)o2)s1)c1cc(C)n(C)n1] | 1H NMR (400 MHz, CDCl3) δ ppm 8.40 (s, 1 H), 6.91 (t, J = 51.6 Hz, 1 H), 6.13 (s, 1 H), 5.37 (s, 2 H), 3.70 (s, 3 H), 3.27 (q, J = 7.6 Hz, 2 H), 2.24 (s, 3 H), 1.42 (t, J = 7.2 Hz, 3 H). LCMS RT = 3.486 min, m/z = 418.9 | 0.117 |
| | N-((5-(5-(difluoromethyl)-1,3,4- | | |
| | oxadiazol-2-yl)thiazol-2- | | |
| | yl)methyl)-N-(1,5-dimethyl-1H- | | |
| | pyrazol-3-yl)ethanesulfonamide | | |
| I-58 | 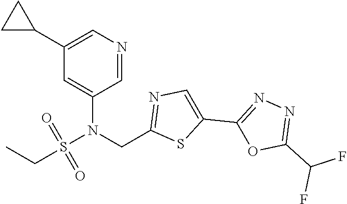 [SMILES: CCS(=O)(=O)N(Cc1ncc(-c2nnc(C(F)F)o2)s1)c1cncc(C2CC2)c1] | 1H NMR (400 MHz, CDCl3) δ ppm 8.45 (s, 1 H), 8.41 (s, 1 H), 8.35 (s, 1 H), 7.46 (s, 1 H), 6.91 (t, J = 51.6 Hz, 1 H), 5.27 (s, 2 H), 3.19 (q, J = 8.0 Hz, 2 H), 1.92-1.89 (m, 1 H), 1.45 (t, J = 7.2 Hz, 3 H), 1.11-1.05 (m, 2 H), 0.76-0.74 (m, 2 H). LCMS RT = 0.992 min, m/z = 442.3 | 0.025 |
| | N-(5-cyclopropylpyridin-3-yl)-N- | | |
| | ((5-(5-(difluoromethyl)-1,3,4- | | |
| | oxadiazol-2-yl)thiazol-2- | | |
| | yl)methyl)ethanesulfonamide | | |
| I-59 | 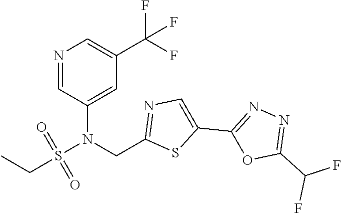 [SMILES: CCS(=O)(=O)N(Cc1ncc(-c2nnc(C(F)F)o2)s1)c1cncc(C(F)(F)F)c1] | 1H NMR (400 MHz, CDCl3) δ ppm 8.93 (d, J = 2.0 Hz, 1 H), 8.83 (s, 1 H), 8.41 (s, 1 H), 8.10 (s, 1 H), 6.91 (t, J = 51.6 Hz, 1 H), 5.31 (s, 2 H), 3.22 (q, J = 7.6 Hz, 2 H), 1.45 (t, J = 7.6 Hz, 3 H). LCMS RT = 1.190 min, m/z = 469.8 | 0.030 |
| | N-((5-(5-(difluoromethyl)-1,3,4- | | |
| | oxadiazol-2-yl)thiazol-2- | | |
| | yl)methyl)-N-(5- | | |
| | (trifluoromethyl)pyridin-3- | | |
| | yl)ethanesulfonamide | | |
| I-60 | 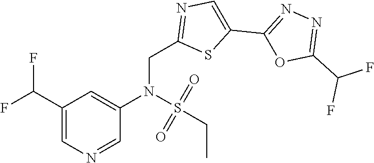 [SMILES: CCS(=O)(=O)N(Cc1ncc(-c2nnc(C(F)F)o2)s1)c1cncc(C(F)F)c1] | 1H NMR (400 MHz, CDCl3) δ ppm 8.85 (s, 1 H), 8.73 (s, 1 H), 8.40 (s, 1 H), 7.99 (s, 1 H), 6.91 (t, J = 52.0 Hz, 1 H), 6.74 (t, J = 56.0 Hz, 1 H), 5.31 (s, 2 H), 3.22 (q, J = 4.0 Hz, 2 H), 1.46 (t, J = 8.0 Hz, 3 H). LCMS RT = 2.508 min, m/z = 452.1 | 0.070 |
| | N-((5-(5-(difluoromethyl)-1,3,4- | | |
| | oxadiazol-2-yl)thiazol-2- | | |
| | yl)methyl)-N-(5- | | |
| | (difluoromethyl)pyridin-3- | | |
| | yl)ethanesulfonamide | | |
| I-61 | 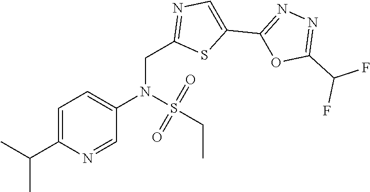 [SMILES: CCS(=O)(=O)N(Cc1ncc(-c2nnc(C(F)F)o2)s1)c1ccc(C(C)C)nc1] | 1H NMR (400 MHz, CDCl3) δ ppm 8.59 (d, J = 2.4 Hz, 1 H), 8.38 (s, 1 H), 7.75-7.71 (m, 1 H), 7.22 (d, J = 8.0 Hz, 1H), 6.91 (t, J = 51.6 Hz, 1 H), 5.27 (s, 2 H), 3.20 (q, J = 7.2 Hz, 2 H), 3.11-3.03 (m, 1 H), 1.47 (t, J = 7.2 Hz, 3 H), 1.29 (d, J = 7.2 Hz, 6 H). LCMS RT = 0.757 min, m/z = 444.2 | 0.073 |
| | N-((5-(5-(difluoromethyl)-1,3,4- | | |
| | oxadiazol-2-yl)thiazol-2- | | |
| | yl)methyl)-N-(6-isopropylpyridin- | | |
| | 3-yl)ethanesulfonamide | | |
| I-62 | 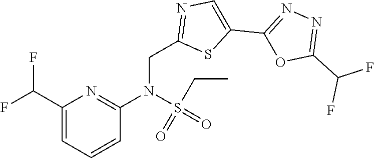 [SMILES: CCS(=O)(=O)N(Cc1ncc(-c2nnc(C(F)F)o2)s1)c1cccc(C(F)F)n1] | 1H NMR (400 MHz, CDCl3) δ ppm 8.30 (s, 1 H), 7.85-7.69 (m, 2 H), 7.39 (d, J = 7.6 Hz, 1 H), 6.81 (t, J = 51.6 Hz, 1 H), 6.47 (t, J = 79.2 Hz, 1 H), 5.54 (s, 2 H), 3.29 (q, J = 7.6 Hz, 2 H), 1.33 (t, J = 7.6 Hz, 3 H). LCMS RT = 2.238 min, m/z = 452.1 | 0.0206 |
| | N-((5-(5-(difluoromethyl)-1,3,4- | | |
| | oxadiazol-2-yl)thiazol-2- | | |
| | yl)methyl)-N-(6- | | |
| | (difluoromethyl)pyridin-2- | | |
| | yl)ethanesulfonamide | | |
| I-63 | 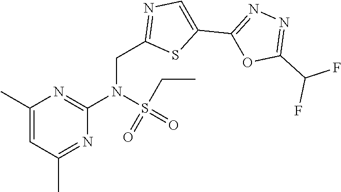 [SMILES: CCS(=O)(=O)N(Cc1ncc(-c2nnc(C(F)F)o2)s1)c1nc(C)cc(C)n1] | 1H NMR (400 MHz, CDCl3) δ ppm 8.40 (s, 1 H), 6.89 (t, J = 51.6 Hz, 1 H), 6.77 (s, 1 H), 5.65 (s, 2 H), 3.95 (q, J = 7.6 Hz, 2 H), 2.44 (s, 6 H), 1.39 (t, J = 7.6 Hz, 3 H). LCMS RT = 3.758 min, m/z = 431.2 | 0.018 |
| | N-((5-(5-(difluoromethyl)-1,3,4- | | |
| | oxadiazol-2-yl)thiazol-2- | | |
| | yl)methyl)-N-(4,6- | | |
| | dimethylpyrimidin-2- | | |
| | yl)ethanesulfonamide | | |
| I-64 | 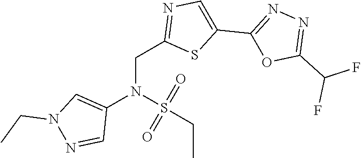 | 1H NMR (400 MHz, CDCl3) δ ppm 8.40 (s, 1 H), 7.60 (s, 1 H), 7.49 (s, 1 H), 6.91 (t, J = 51.6 Hz, 1 H), 5.16 (s, 2 H), 4.12 (q, J = 7.2 Hz, 2 H), 3.17 (q, J = 7.6 Hz, 2 H), 1.50- 1.40 (m, 6 H). LCMS RT = 1.418 min, m/z = 418.9 | 0.065 |
| | N-((5-(5-(difluoromethyl)-1,3,4- | | |
| | oxadiazol-2-yl)thiazol-2- | | |
| | yl)methyl)-N-(1-ethyl-1H-pyrazol- | | |
| | 4-yl)ethanesulfonamide | | |
| I-65 | 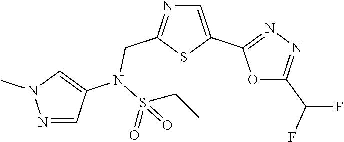 [SMILES: CCS(=O)(=O)N(Cc1ncc(-c2nnc(C(F)F)o2)s1)c1cnn(C)c1] | 1H NMR (400 MHz, CD3OD) δ ppm 8.33 (s, 1 H), 7.71 (s, 1 H), 7.48 (s, 1 H), 7.11 (t, J = 51.6 Hz, 1 H), 5.10 (s, 2 H), 3.74 (s, 3 H), 3.14 (q, J = 7.2 Hz, 2 H), 1.28 (t, J = 7.2 Hz, 3 H). LCMS RT = 2.950 min, m/z = 404.9 | 0.022 |
| | N-((5-(5-(difluoromethyl)-1,3,4- | | |
| | oxadiazol-2-yl)thiazol-2- | | |
| | yl)methyl)-N-(1-methyl-1H- | | |
| | pyrazol-4-yl)ethanesulfonamide | | |
| I-66 | 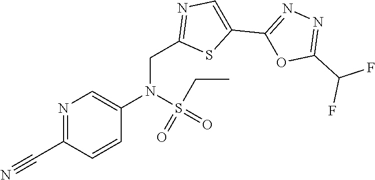 [SMILES: CCS(=O)(=O)N(Cc1ncc(-c2nnc(C(F)F)o2)s1)c1ccc(C#N)nc1] | 1H NMR (400 MHz, CDCl3) δ ppm 8.87 (s, 1 H), 8.40 (s, 1 H), 8.04 (d, J = 8.4 Hz, 1 H), 7.74 (d, J = 8.4 Hz, 1 H), 6.91 (t, J = 51.6 Hz, 1 H), 5.35 (s, 2 H), 3.23 (q, J = 7.2 Hz, 2 H), 1.43 (t, J = 7.2 Hz, 3 H). LCMS RT = 1.030 min, m/z = 426.8 | 0.029 |
| | N-(6-cyanopyridin-3-yl)-N-((5-(5- | | |
| | (difluoromethyl)-1,3,4-oxadiazol- | | |
| | 2-yl)thiazol-2- | | |
| | yl)methyl)ethanesulfonamide | | |
| I-67 | 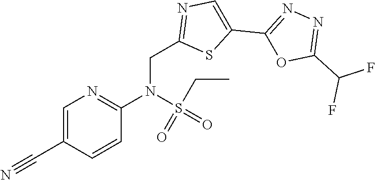 [SMILES: CCS(=O)(=O)N(Cc1ncc(-c2nnc(C(F)F)o2)s1)c1ccc(C#N)cn1] | 1H NMR (400 MHz, CDCl3) δ ppm 8.66 (s, 1 H), 8.39 (s, 1 H), 7.96-7.78 (m, 2 H), 6.90 (t, J = 51.6 Hz, 1 H), 5.63 (s, 2 H), 3.48 (q, J = 7.6 Hz, 2 H), 1.43 (t, J = 7.6 Hz, 3 H). LCMS RT = 2.180 min, m/z = 427.1 | 0.037 |
| | N-(5-cyanopyridin-2-yl)-N-((5-(5- | | |
| | (difluoromethyl)-1,3,4-oxadiazol- | | |
| | 2-yl)thiazol-2- | | |
| | yl)methyl)ethanesulfonamide | | |
| I-68 | 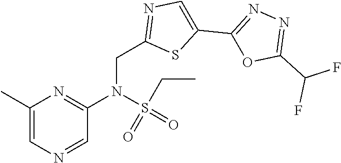 [SMILES: CCS(=O)(=O)N(Cc1ncc(-c2nnc(C(F)F)o2)s1)c1cncc(C)n1] | 1H NMR (400 MHz, CDCl3) δ ppm 8.80 (s, 1 H), 8.39 (s, 1 H), 8.29 (s, 1 H), 6.90 (t, J = 51.6 Hz, 1 H), 5.58 (s, 2 H), 3.38 (q, J = 7.2 Hz, 2 H), 2.55 (s, 3 H), 1.42 (t, J = 7.6 Hz, 3 H). LCMS RT = 1.657 min, m/z = 417.1 | 0.100 |
| | N-((5-(5-(difluoromethyl)-1,3,4- | | |
| | oxadiazol-2-yl)thiazol-2- | | |
| | yl)methyl)-N-(6-methylpyrazin-2- | | |
| | yl)ethanesulfonamide | | |
| I-69 | 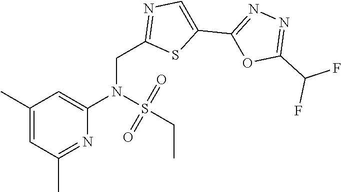 [SMILES: CCS(=O)(=O)N(Cc1ncc(-c2nnc(C(F)F)o2)s1)c1cc(C)cc(C)n1] | 1H NMR (400 MHz, CDCl3) δ ppm 8.38 (s, 1 H), 7.27 (s, 1 H), 7.02-6.72 (m, 2 H), 5.58 (s, 2 H), 3.24 (q, J = 7.6 Hz, 2 H), 2.47 (s, 3 H), 2.33 (s, 3 H), 1.36 (t, J = 7.4 Hz, 3 H). LCMS RT = 3.082 min, m/z = 429.9 | 0.071 |
| | N-((5-(5-(difluoromethyl)-1,3,4- | | |
| | oxadiazol-2-yl)thiazol-2- | | |
| | yl)methyl)-N-(4,6- | | |
| | dimethylpyridin-2- | | |
| | yl)ethanesulfonamide | | |
| I-70 | 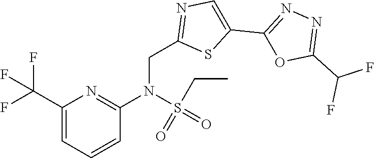 [SMILES: CCS(=O)(=O)N(Cc1ncc(-c2nnc(C(F)F)o2)s1)c1cccc(C(F)(F)F)n1] | 1H NMR (400 MHz, CD3OD) δ ppm 8.42 (s, 1 H), 8.07- 8.02 (m, 1 H), 7.92-7.89 (m, 1 H), 7.61-7.58 (m, 1 H), 7.19 (t, J = 51.6 Hz, 1 H), 5.62 (s, 2 H), 3.57 (q, J = 7.2 Hz, 2 H), 1.38 (t, J = 7.2 Hz, 3 H). LCMS RT = 2.965 min, m/z = 470.1 | 0.0281 |
| | N-((5-(5-(difluoromethyl)-1,3,4- | | |
| | oxadiazol-2-yl)thiazol-2- | | |
| | yl)methyl)-N-(6- | | |
| | (trifluoromethyl)pyridin-2- | | |
| | yl)ethanesulfonamide | | |
| I-71 | 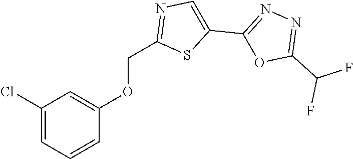 [SMILES: FC(F)c1nnc(-c2cnc(COc3cccc(Cl)c3)s2)o1] | 1H NMR (400 MHz, CDCl3) δ ppm 8.49 (s, 1 H), 7.29-7.24 (m, 1 H), 7.06-7.04 (m, 2 H), 6.94-6.78 (m, 2 H), 5.43 (s, 2 H). LCMS RT = 1.840 min, m/z = 344.1 | 0.251 |
| | 2-(2-((3- | | |
| | chlorophenoxy)methyl)thiazol-5- | | |
| | yl)-5-(difluoromethyl)-1,3,4- | | |
| | oxadiazole | | |
| I-72 | 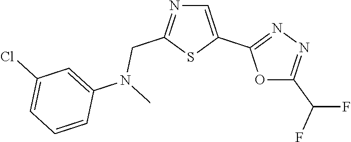 [SMILES: CN(Cc1ncc(-c2nnc(C(F)F)o2)s1)c1cccc(Cl)c1] | 1H NMR (400 MHz, CDCl3) δ ppm 8.55 (s, 1 H), 7.43-6.70 (m, 5 H), 4.93 (s, 2 H), 3.26 (s, 3 H). LCMS RT = 3.547 min, m/z = 356.8 | 0.034 |
| | 3-chloro-N-((5-(5- | | |
| | (difluoromethyl)-1,3,4-oxadiazol- | | |
| | 2-yl)thiazol-2-yl)methyl)-N- | | |
| | methylaniline | | |
| I-73 | 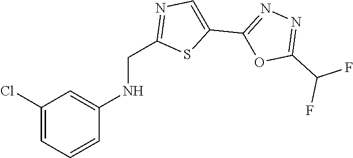 [SMILES: FC(F)c1nnc(-c2cnc(CNc3cccc(Cl)c3)s2)o1] | 1H NMR (400 MHz, CDCl3) δ ppm 8.37 (s, 1 H), 7.06-6.44 (m, 5 H), 4.66 (s, 3 H). LCMS RT = 2.368 min, m/z = 343.1 | 0.046 |
| | 3-chloro-N-((5-(5- | | |
| | (difluoromethyl)-1,3,4-oxadiazol- | | |
| | 2-yl)thiazol-2-yl)methyl)aniline | | |
| I-74 | 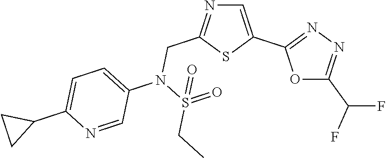 [SMILES: CCS(=O)(=O)N(Cc1ncc(-c2nnc(C(F)F)o2)s1)c1ccc(C2CC2)nc1] | 1H NMR (400 MHz, CD3OD) δ ppm 8.46 (d, J = 2.4 Hz, 1 H), 8.41 (s, 1 H), 7.80 (dd, J = 8.4, 2.8 Hz, 1 H), 7.33-7.07 (m, 2 H), 5.33 (s, 2 H), 3.31- 3.27 (m, 2 H), 2.11-2.08 (m, 1 H), 1.40 (t, J = 7.2 Hz, 3 H), 1.04-0.94 (m, 4 H). LCMS RT = 5.273 min, m/z = 441.9 | 0.016 |
| | N-(6-cyclopropylpyridin-3-yl)-N- | | |
| | ((5-(5-(difluoromethyl)-1,3,4- | | |
| | oxadiazol-2-yl)thiazol-2- | | |
| | yl)methyl)ethanesulfonamide | | |
| I-75 | 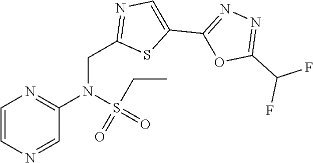 [SMILES: CCS(=O)(=O)N(Cc1ncc(-c2nnc(C(F)F)o2)s1)c1cnccn1] | 1H NMR (400 MHz, CDCl3) δ ppm 9.00 (s, 1 H), 8.43 (d, J = 2.8 Hz, 1 H), 8.39-8.35 (m, 2 H), 6.89 (t, J = 51.6 Hz, 1 H), 5.58 (s, 2 H), 3.41 (q, J = 7.6 Hz, 2 H), 1.43 (t, J = 7.2 Hz, 3 H). LCMS RT = 2.823 min, m/z = 403.1 | 0.023 |
| | N-((5-(5-(difluoromethyl)-1,3,4- | | |
| | oxadiazol-2-yl)thiazol-2- | | |
| | yl)methyl)-N-(pyrazin-2- | | |
| | yl)ethanesulfonamide | | |
| I-76 | 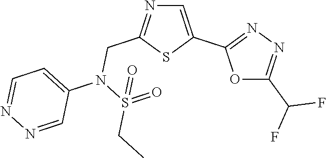 [SMILES: CCS(=O)(=O)N(Cc1ncc(-c2nnc(C(F)F)o2)s1)c1ccnnc1] | 1H NMR (400 MHz, CD3OD) δ ppm 8.57 (d, J = 7.6 Hz, 1 H), 8.53 (s, 1 H), 8.23 (s, 1 H), 7.42-7.40 (m, 1 H), 7.22 (t, J = 51.6 Hz, 1 H), 5.85 (s, 2 H), 3.10 (q, J = 7.6 Hz, 2 H), 1.36 (t, J = 7.2 Hz, 3 H). LCMS RT = 3.671 min, m/z = 402.9 | 0.097 |
| | N-((5-(5-(difluoromethyl)-1,3,4- | | |
| | oxadiazol-2-yl)thiazol-2- | | |
| | yl)methyl)-N-(pyridazin-4- | | |
| | yl)ethanesulfonamide | | |
| I-77 | 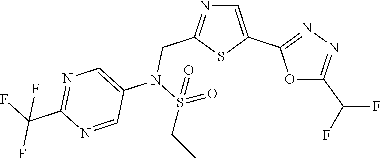 [SMILES: CCS(=O)(=O)N(Cc1ncc(-c2nnc(C(F)F)o2)s1)c1cnc(C(F)(F)F)nc1] | 1H NMR (400 MHz, CDCl3) δ ppm 9.10 (s, 2 H), 8.43 (s, 1 H), 6.92 (t, J = 51.6 Hz, 1 H), 5.32 (d, J = 12 Hz, 2 H), 3.25 (q, J = 7.2 Hz, 2 H), 1.44 (t, J = 7.2 Hz, 3 H). LCMS RT = 2.594 min, m/z = 471.1 | 0.155 |
| | N-((5-(5-(difluoromethyl)-1,3,4- | | |
| | oxadiazol-2-yl)thiazol-2- | | |
| | yl)methyl)-N-(2- | | |
| | (trifluoromethyl)pyrimidin-5- | | |
| | yl)ethanesulfonamide | | |
| I-78 | 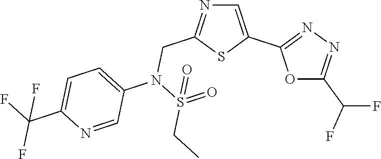 [SMILES: CCS(=O)(=O)N(Cc1ncc(-c2nnc(C(F)F)o2)s1)c1ccc(C(F)(F)F)nc1] | 1H NMR (400 MHz, CDCl3) δ ppm 8.86 (s, 1 H), 8.40 (s, 1 H), 8.10 (d, J = 8.4 Hz, 1 H), 7.74 (d, J = 8.4 Hz, 1 H), 6.91 (t, J = 51.6 Hz, 1 H), 5.34 (s, 2 H), 3.23 (q, J = 7.2 Hz, 2 H), 1.45 (t, J = 7.2 Hz, 3 H). LCMS RT = 0.882 min, m/z = 470.1 | 0.059 |
| | N-((5-(5-(difluoromethyl)-1,3,4- | | |
| | oxadiazol-2-yl)thiazol-2- | | |
| | yl)methyl)-N-(6- | | |
| | (trifluoromethyl)pyridin-3- | | |
| | yl)ethanesulfonamide | | |
| I-79 | 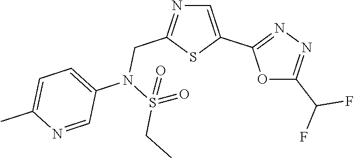 [SMILES: CCS(=O)(=O)N(Cc1ncc(-c2nnc(C(F)F)o2)s1)c1ccc(C)nc1] | 1H NMR (400 MHz, CDCl3) δ ppm 8.55 (s, 1 H), 8.37 (s, 1 H), 7.73-7.68 (m, 1 H), 7.20 (d, J = 7.6 Hz, 1 H), 6.91 (t, J = 51.6 Hz, 1 H), 5.27 (s, 2 H), 3.20 (q, J = 7.2 Hz, 2 H), 2.56 (s, 3 H), 1.46 (t, J = 7.2 Hz, 3 H). LCMS RT = 0.616 min, m/z = 416.2 | 0.073 |
| | N-((5-(5-(difluoromethyl)-1,3,4- | | |
| | oxadiazol-2-yl)thiazol-2- | | |
| | yl)methyl)-N-(6-methylpyridin-3- | | |
| | yl)ethanesulfonamide | | |
| I-80 | 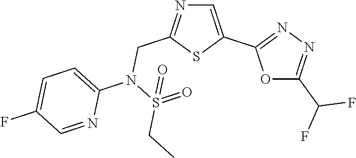 [SMILES: CCS(=O)(=O)N(Cc1ncc(-c2nnc(C(F)F)o2)s1)c1ccc(F)cn1] | 1H NMR (400 MHz, CDCl3) δ ppm 8.37 (s, 1 H), 8.28 (s, 1 H), 7.70-7.60 (m, 1 H), 7.50- 7.40 (m, 1 H), 6.89 (t, J = 51.6 Hz, 1 H), 5.54 (s, 2 H), 3.25 (q, J = 7.2 Hz, 2 H), 1.40 (t, J = 7.6 Hz, 3 H). LCMS RT = 2.661 min, m/z = 419.9 | 0.0265 |
| | N-((5-(5-(difluoromethyl)-1,3,4- | | |
| | oxadiazol-2-yl)thiazol-2- | | |
| | yl)methyl)-N-(5-fluoropyridin-2- | | |
| | yl)ethanesulfonamide | | |
| I-81 | 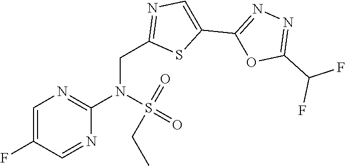 [SMILES: CCS(=O)(=O)N(Cc1ncc(-c2nnc(C(F)F)o2)s1)c1ncc(F)cn1] | 1H NMR (400 MHz, CDCl3) δ ppm 8.49 (s, 2 H), 8.40 (s, 1 H), 6.90 (t, J = 51.6 Hz, 1 H), 5.65 (s, 2 H), 3.91 (q, J = 7.6 Hz, 2 H), 1.45 (t, J = 7.6 Hz, 3 H). LCMS RT = 1.977 min, m/z = 421.1 | 0.116 |
| | N-((5-(5-(difluoromethyl)-1,3,4- | | |
| | oxadiazol-2-yl)thiazol-2- | | |
| | yl)methyl)-N-(5-fluoropyrimidin- | | |
| | 2-yl)ethanesulfonamide | | |
| I-82 | 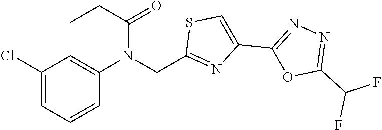 [SMILES: CCC(=O)N(Cc1nc(-c2nnc(C(F)F)o2)cs1)c1cccc(Cl)c1] | 1H NMR (400 MHz, CDCl3) δ ppm 8.28 (s, 1 H), 7.37-7.32 (m, 2 H), 7.25-7.23 (m, 1 H), 7.10-7.07 (m, 1 H), 6.91 (t, J = 51.6 Hz, 1 H), 5.22 (s, 2 H), 2.19 (q, J = 7.2 Hz, 2 H), 1.21 (t, J = 7.2 Hz, 3 H). LCMS RT = 0.931 min, m/z = 399.1 | 4.1 |
| | N-(3-chlorophenyl)-N-((4-(5- | | |
| | (difluoromethyl)-1,3,4-oxadiazol- | | |
| | 2-yl)thiazol-2- | | |
| | yl)methyl)propionamide | | |
| I-83 | 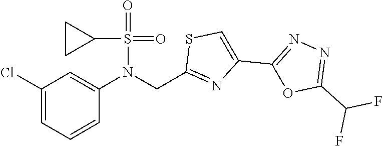 [SMILES: O=S(=O)(C1CC1)N(Cc1nc(-c2nnc(C(F)F)o2)cs1)c1cccc(Cl)c1] | 1H NMR (400 MHz, CDCl3) δ ppm 8.26 (s, 1 H), 7.51 (s, 1 H), 7.41-7.30 (m, 3 H), 6.91 (t, J = 51.6 Hz, 1 H), 5.32 (s, 2 H), 2.59-2.52 (m, 1 H), 1.18- 1.13 (m, 2 H), 1.08-1.04 (m, 2 H). LCMS RT = 0.944 min, m/z = 447.1 | 0.759 |
| | N-(3-chlorophenyl)-N-((4-(5- | | |
| | (difluoromethyl)-1,3,4-oxadiazol- | | |
| | 2-yl)thiazol-2- | | |
| | yl)methyl)cyclopropanesulfonamide | | |
| I-84 | 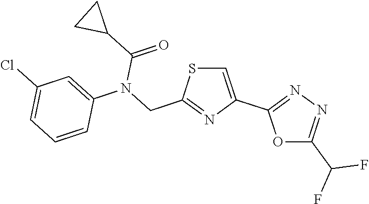 [SMILES: O=C(C1CC1)N(Cc1nc(-c2nnc(C(F)F)o2)cs1)c1cccc(Cl)c1] | 1H NMR (400 MHz, CDCl3) δ ppm 8.27 (s, 1 H), 7.40-7.20 (m, 4 H), 6.91 (t, J = 51.6 Hz, 1 H), 5.25 (s, 2 H), 1.50-1.40 (m, 1 H), 1.18-1.09 (m, 2 H), 0.83-0.74 (m, 2 H). LCMS RT = 1.163 min, m/z = 410.8 | 7.5 |
| | N-(3-chlorophenyl)-N-((4-(5- | | |
| | (difluoromethyl)-1,3,4-oxadiazol- | | |
| | 2-yl)thiazol-2- | | |
| | yl)methyl)cyclopropanecarboxamide | | |
| I-85 | 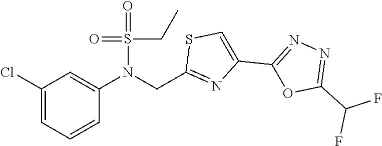 [SMILES: CCS(=O)(=O)N(Cc1nc(-c2nnc(C(F)F)o2)cs1)c1cccc(Cl)c1] | 1H NMR (400 MHz, CDCl3) δ ppm 8.26 (s, 1 H), 7.48 (s, 1 H), 7.37-7.31 (m, 3 H), 6.91 (t, J = 51.6 Hz, 1 H), 5.32 (s, 2 H), 3.19 (q, J = 7.2 Hz, 2 H), 1.46 (t, J = 7.2 Hz, 3 H). LCMS RT = 0.922 min, m/z = 435.0 | 0.584 |
| | N-(3-chlorophenyl)-N-((4-(5- | | |
| | (difluoromethyl)-1,3,4-oxadiazol- | | |
| | 2-yl)thiazol-2- | | |
| | yl)methyl)ethanesulfonamide | | |
| I-86 | 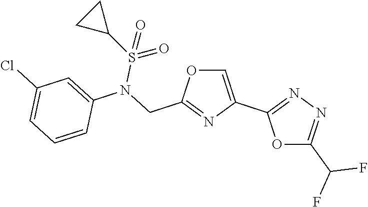 [SMILES: O=S(=O)(C1CC1)N(Cc1nc(-c2nnc(C(F)F)o2)co1)c1cccc(Cl)c1] | 1H NMR (400 MHz, CDCl3) δ ppm 8.36 (s, 1 H), 7.50 (s, 1 H), 7.39-7.37 (m, 1 H), 7.33- 7.30 (m, 2 H), 6.91 (t, J = 51.6 Hz, 1 H), 5.03 (s, 2 H), 2.69-2.65 (m, 1 H), 1.09- 0.90 (m, 4 H). LCMS RT = 3.210 min, m/z = 431.1 | 8.0 |
| | N-(3-chlorophenyl)-N-((4-(5- | | |
| | (difluoromethyl)-1,3,4-oxadiazol- | | |
| | 2-yl)oxazol-2- | | |
| | yl)methyl)cyclopropanesulfonamide | | |
| I-87 | 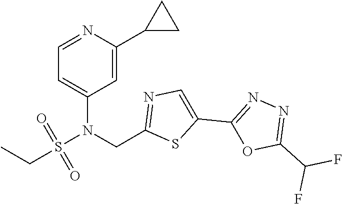 [SMILES: CCS(=O)(=O)N(Cc1ncc(-c2nnc(C(F)F)o2)s1)c1ccnc(C2CC2)c1] | 1H NMR (400 MHz, CDCl3) δ 8.42-8.36 (m, 2 H), 7.31 (s, 1 H), 7.18-7.14 (m, 1 H), 6.90 (t, J = 51.6 Hz, 1 H), 5.37 (s, 2 H), 3.26 (q, J = 7.6 Hz, 2 H), 2.04-1.95 (m, 1 H), 1.42 (t, J = 7.2 Hz, 3 H), 1.05-1.00 (m, 4 H). LCMS RT = 1.094 min, m/z = 441.9 | 0.039 |
| | N-(2-cyclopropylpyridin-4-yl)-N- | | |
| | ((5-(5-(difluoromethyl)-1,3,4- | | |
| | oxadiazol-2-yl)thiazol-2- | | |
| | yl)methyl)ethanesulfonamide | | |
| I-88 | 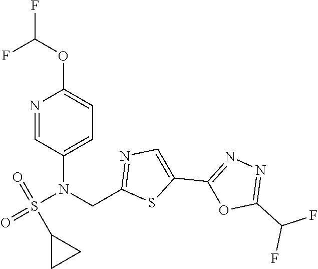 [SMILES: O=S(=O)(C1CC1)N(Cc1ncc(-c2nnc(C(F)F)o2)s1)c1ccc(OC(F)F)nc1] | 1H NMR (400 MHz, CDCl3) δ 8.39 (s, 1 H), 8.33 (d, J = 2.8 Hz, 1 H), 7.87 (dd, J = 2.8, 8.8 Hz, 1 H), 7.42 (t, J = 72.0 Hz, 1 H), 7.06-6.77 (m, 2 H), 5.26 (s, 2 H), 2.64-2.48 (m, 1 H), 1.21-0.98 (m, 4 H). LCMS RT = 0.957 min, m/z = 479.9 | 0.048 |
| | N-(6-(difluoromethoxy)pyridin-3- | | |
| | yl)-N-((5-(5-(difluoromethyl)- | | |
| | 1,3,4-oxadiazol-2-yl)thiazol-2- | | |
| | yl)methyl)cyclopropanesulfonamide | | |
| I-89 | 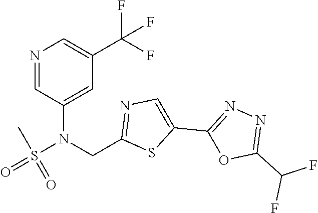 [SMILES: CS(=O)(=O)N(Cc1ncc(-c2nnc(C(F)F)o2)s1)c1cncc(C(F)(F)F)c1] | 1H NMR (400 MHz, CDCl3) δ 8.95 (d, J = 2.4 Hz, 1 H), 8.87 (d, J = 2.4 Hz, 1 H), 8.44 (s, 1 H), 8.13 (s, 1 H), 6.92 (t, J = 51.6 Hz, 1 H), 5.31 (s, 2 H), 3.12 (s, 3 H). LCMS RT = 0.878 min, m/z = 455.9 | 0.049 |
| | N-((5-(5-(difluoromethyl)-1,3,4- | | |
| | oxadiazol-2-yl)thiazol-2- | | |
| | yl)methyl)-N-(5- | | |
| | (trifluoromethyl)pyridin-3- | | |
| | yl)methanesulfonamide | | |
| I-90 | 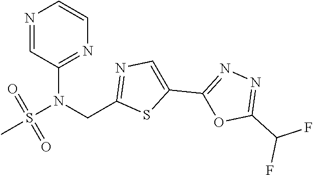 [SMILES: CS(=O)(=O)N(Cc1ncc(-c2nnc(C(F)F)o2)s1)c1cnccn1] | 1H NMR (400 MHz, CDCl3) δ 9.00 (s, 1 H), 8.47-8.36 (m, 3 H), 6.91 (t, J = 51.2 Hz, 1 H), 5.58 (s, 2 H), 3.27 (s, 3 H). LCMS RT = 0.943 min, m/z = 389.1 | 0.041 |
| | N-((5-(5-(difluoromethyl)-1,3,4- | | |
| | oxadiazol-2-yl)thiazol-2- | | |
| | yl)methyl)-N-(pyrazin-2- | | |
| | yl)methanesulfonamide | | |
| I-91 | 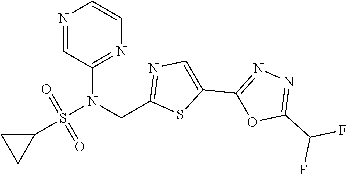 [SMILES: O=S(=O)(C1CC1)N(Cc1ncc(-c2nnc(C(F)F)o2)s1)c1cnccn1] | 1H NMR (400 MHz, CDCl3) δ 9.02 (d, J = 1.2 Hz, 1 H), 8.46- 8.35 (m, 3 H), 6.91 (t, J = 51.6 Hz, 1 H), 5.58 (s, 2 H), 2.72-2.61 (m, 1 H), 1.27- 1.21 (m, 2 H), 1.11-1.01 (m, 2 H). LCMS RT = 1.031 min, m/z = 415.1 | 0.018 |
| | N-((5-(5-(difluoromethyl)-1,3,4- | | |
| | oxadiazol-2-yl)thiazol-2- | | |
| | yl)methyl)-N-(pyrazin-2- | | |
| | yl)cyclopropanesulfonamide | | |
| I-92 | 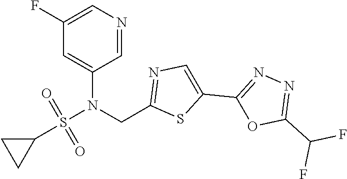 [SMILES: O=S(=O)(C1CC1)N(Cc1ncc(-c2nnc(C(F)F)o2)s1)c1cncc(F)c1] | 1H NMR (400 MHz, CDCl3) δ 8.61 (s, 1 H), 8.47 (d, J = 4.0 Hz, 1 H), 8.40 (s, 1 H), 7.68- 7.62 (m, 1 H), 6.91 (t, J = 51.6 Hz, 1 H), 5.31 (s, 2 H), 2.63- 2.52 (m, 1 H), 1.17-1.02 (m, 4 H). LCMS RT = 2.295 min, m/z = 431.9 | 0.014 |
| | N-((5-(5-(difluoromethyl)-1,3,4- | | |
| | oxadiazol-2-yl)thiazol-2- | | |
| | yl)methyl)-N-(5-fluoropyridin-3- | | |
| | yl)cyclopropanesulfonamide | | |
| I-93 | 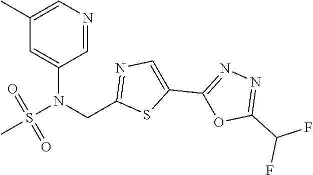 [SMILES: Cc1cncc(N(Cc2ncc(-c3nnc(C(F)F)o3)s2)S(C)(=O)=O)c1] | 1H NMR (400 MHz, CDCl3) δ 8.52-8.43 (m, 2 H), 8.40 (s, 1 H), 7.65 (s, 1 H), 6.91 (t, J = 51.6 Hz, 1 H), 5.27 (s, 2 H), 3.10 (s, 3 H), 2.39 (s, 3 H). LCMS RT = 0.828 min, m/z = 401.9 | 0.055 |
| | N-((5-(5-(difluoromethyl)-1,3,4- | | |
| | oxadiazol-2-yl)thiazol-2- | | |
| | yl)methyl)-N-(5-methylpyridin-3- | | |
| | yl)methanesulfonamide | | |
| I-94 | 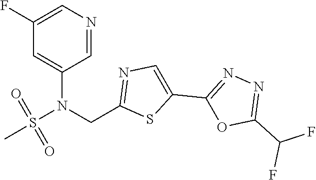 [SMILES: CS(=O)(=O)N(Cc1ncc(-c2nnc(C(F)F)o2)s1)c1cncc(F)c1] | 1H NMR (400 MHz, CDCl3) δ 8.58 (s, 1 H), 8.49 (s, 1 H), 8.43 (s, 1 H), 7.68-7.65 (m, 1 H), 6.92 (t, J = 51.6 Hz, 1 H), 5.29 (s, 2 H), 3.12 (s, 3 H). LCMS RT = 1.877 min, m/z = 406.1 | 0.025 |
| | N-((5-(5-(difluoromethyl)-1,3,4- | | |
| | oxadiazol-2-yl)thiazol-2- | | |
| | yl)methyl)-N-(5-fluoropyridin-3- | | |
| | yl)methanesulfonamide | | |
| I-95 | 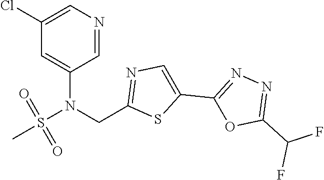 [SMILES: CS(=O)(=O)N(Cc1ncc(-c2nnc(C(F)F)o2)s1)c1cncc(Cl)c1] | 1H NMR (400 MHz, CDCl3) δ 8.62 (d, J = 2.4 Hz, 1 H), 8.57 (d, J = 2.4 Hz, 1 H), 8.43 (s, 1 H), 7.90 (s, 1 H), 6.92 (t, J = 51.6 Hz, 1 H), 5.27 (s, 2 H), 3.11 (s, 3 H). LCMS RT = 2.188 min, m/z = 421.8 | 0.0235 |
| | N-(5-chloropyridin-3-yl)-N-((5-(5- | | |
| | (difluoromethyl)-1,3,4-oxadiazol- | | |
| | 2-yl)thiazol-2- | | |
| | yl)methyl)methanesulfonamide | | |
| I-96 | 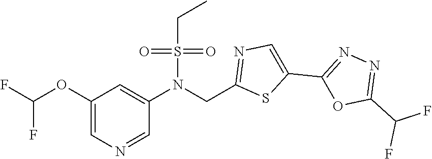 [SMILES: CCS(=O)(=O)N(Cc1ncc(-c2nnc(C(F)F)o2)s1)c1cncc(OC(F)F)c1] | 1H NMR (400 MHz, CDCl3) δ 8.63 (s, 1 H), 8.46 (s, 1 H), 8.39 (s, 1 H), 7.68 (s, 1 H), 6.91 (t, J = 51.6 Hz, 1 H), 6.58 (t, J = 51.6 Hz, 1 H), 5.29 (s, 2 H), 3.22 (q, J = 7.2 Hz , 2 H), 1.44 (t, J = 7.6 Hz, 3 H). LCMS RT = 0.907 min, m/z = 467.8 | 0.010 |
| | N-(5-(difluoromethoxy)pyridin-3- | | |
| | yl)-N-((5-(5-(difluoromethyl)- | | |
| | 1,3,4-oxadiazol-2-yl)thiazol-2- | | |
| | yl)methyl)ethanesulfonamide | | |
| I-97 | 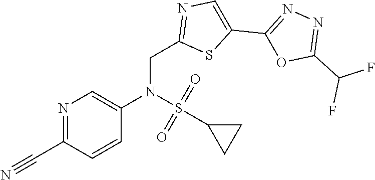 [SMILES: N#Cc1ccc(N(Cc2ncc(-c3nnc(C(F)F)o3)s2)S(=O)(=O)C2CC2)cn1] | 1H NMR (400 MHz, CDCl3) δ 8.89 (d, J = 2.4 Hz, 1 H), 8.39 (s, 1 H), 8.05 (d, J = 8.4 Hz, 1 H), 7.74 (d, J = 8.8 Hz, 1 H), 6.91 (t, J = 51.6 Hz, 1 H), 5.35 (s, 2 H), 2.59-2.53 (m, 1 H), 1.16-1.07 (m, 4 H). LCMS RT = 0.859 min, m/z = 439.0 | 0.010 |
| | N-(6-cyanopyridin-3-yl)-N-((5-(5- | | |
| | (difluoromethyl)-1,3,4-oxadiazol- | | |
| | 2-yl)thiazol-2- | | |
| | yl)methyl)cyclopropanesulfonamide | | |
| I-98 | 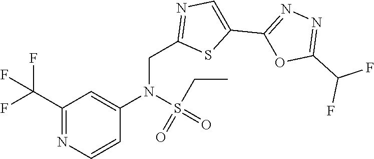 [SMILES: CCS(=O)(=O)N(Cc1ncc(-c2nnc(C(F)F)o2)s1)c1ccnc(C(F)(F)F)c1] | 1H NMR (400 MHz, CDCl3) δ 8.59 (d, J = 5.6 Hz, 1 H), 8.39 (s, 1 H), 7.81 (s, 1 H), 7.61- 7.53 (m, 1 H), 6.83 (t, J = 51.6 Hz, 1 H), 5.35 (s, 2 H), 3.24 (q, J = 7.2 Hz, 2 H), 1.35 (t, J = 7.2 Hz, 3 H). LCMS RT = 0.956 min, m/z = 469.9 | 0.069 |
| | N-((5-(5-(difluoromethyl)-1,3,4- | | |
| | oxadiazol-2-yl)thiazol-2- | | |
| | yl)methyl)-N-(2- | | |
| | (trifluoromethyl)pyridin-4- | | |
| | yl)ethanesulfonamide | | |
| I-99 | 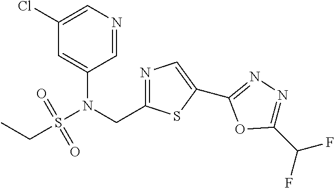 [SMILES: CCS(=O)(=O)N(Cc1ncc(-c2nnc(C(F)F)o2)s1)c1cncc(Cl)c1] | 1H NMR (400 MHz, CDCl3) δ 8.61 (d, J = 2.0 Hz, 1 H), 8.55 (d, J = 2.0 Hz, 1 H), 8.41 (s, 1 H), 7.89 (t, J = 2.2 Hz, 1 H), 6.92 (t, J = 51.6 Hz, 1 H), 5.28 (s, 2 H), 3.22 (q, J = 7.2 Hz, 2 H), 1.46 (t, J = 7.6 Hz, 3 H). LCMS RT = 2.461 min, m/z = 435.9 | 0.0189 |
| | N-(5-chloropyridin-3-yl)-N-((5-(5- | | |
| | (difluoromethyl)-1,3,4-oxadiazol- | | |
| | 2-yl)thiazol-2- | | |
| | yl)methyl)ethanesulfonamide | | |
| I-100 | 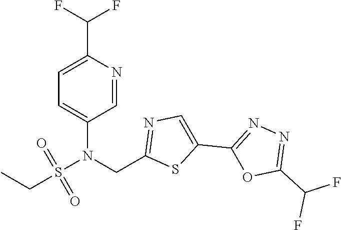 [SMILES: CCS(=O)(=O)N(Cc1ncc(-c2nnc(C(F)F)o2)s1)c1ccc(C(F)F)nc1] | 1H NMR (400 MHz, CDCl3) δ 8.77 (d, J = 2.4 Hz, 1 H), 8.47 (s, 1 H), 8.01 (dd, J = 2.4, 8.4 Hz, 1 H), 7.69 (d, J = 8.4 Hz, 1 H), 6.91 (t, J = 51.6 Hz, 1 H), 6.63 (t, J = 55.2 Hz, 1 H), 5.32 (s, 2 H), 3.21 (q, J = 7.2 Hz, 2 H), 1.45 (t, J = 7.6 Hz, 3 H). LCMS RT = 2.547 min, m/z = 452.1 | 0.065 |
| | N-((5-(5-(difluoromethyl)-1,3,4- | | |
| | oxadiazol-2-yl)thiazol-2- | | |
| | yl)methyl)-N-(6- | | |
| | (difluoromethyl)pyridin-3- | | |
| | yl)ethanesulfonamide | | |
| I-101 | 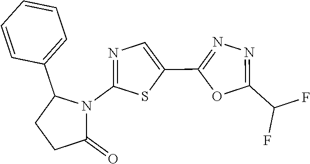 [SMILES: O=C1CCC(c2ccccc2)N1c1ncc(-c2nnc(C(F)F)o2)s1] | 1H NMR (400 MHz, CDCl3) δ 8.08 (s, 1 H), 7.38-7.30 (m, 3 H), 7.18 (d, J = 7.2 Hz, 2 H), 6.88 (t, J = 51.6 Hz, 1 H), 5.80 (d, J = 7.6 Hz, 1 H), 2.93- 2.74 (m, 3 H), 2.25-2.20 (m, 1 H). LCMS RT = 2.937 min, m/z = 362.9 | 0.171 |
| | 1-(5-(5-(difluoromethyl)-1,3,4- | | |
| | oxadiazol-2-yl)thiazol-2-yl)-5- | | |
| | phenylpyrrolidin-2-one | | |
| I-102 | 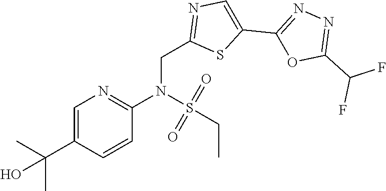 [SMILES: CCS(=O)(=O)N(Cc1ncc(-c2nnc(C(F)F)o2)s1)c1ccc(C(C)(C)O)cn1] | 1H NMR (400 MHz, CDCl3) δ 8.55 (d, J = 2.0 Hz, 1 H), 8.38 (s, 1 H), 7.86 (d, J = 6.8 Hz, 1 H), 7.60 (d, J = 8.8 Hz, 1 H), 6.89 (t, J = 51.6 Hz, 1 H), 5.58 (s, 2 H), 3.29 (q, J = 7.6 Hz , 2 H), 1.61 (s, 6 H), 1.39 (t, J = 7.6 Hz, 3 H). LCMS RT = 1.017 min, m/z = 459.9 | 0.137 |
| | N-((5-(5-(difluoromethyl)-1,3,4- | | |
| | oxadiazol-2-yl)thiazol-2- | | |
| | yl)methyl)-N-(5-(2- | | |
| | hydroxypropan-2-yl)pyridin-2- | | |
| | yl)ethanesulfonamide | | |
| I-103 | 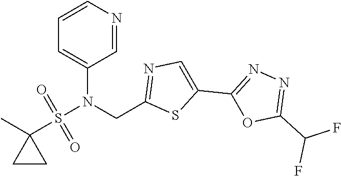 [SMILES: CC1(S(=O)(=O)N(Cc2ncc(-c3nnc(C(F)F)o3)s2)c2cccnc2)CC1] | 1H NMR (400 MHz, CDCl3) δ 8.74 (d, J = 2.4 Hz, 1 H), 8.58 (d, J = 3.6 Hz, 1 H), 8.35 (s, 1 H), 7.87-7.81 (m, 1 H), 7.38- 7.31 (m, 1 H), 6.90 (t, J = 51.6 Hz, 1 H), 5.35 (s, 2 H), 1.59 (s, 3 H), 1.22-1.15 (m, 2 H), 0.79-0.72 (m, 2 H). LCMS RT = 0.956 min, m/z = 427.9 | 0.013 |
| | N-((5-(5-(difluoromethyl)-1,3,4- | | |
| | oxadiazol-2-yl)thiazol-2- | | |
| | yl)methyl)-1-methyl-N-(pyridin-3- | | |
| | yl)cyclopropane-1-sulfonamide | | |
| I-104 | 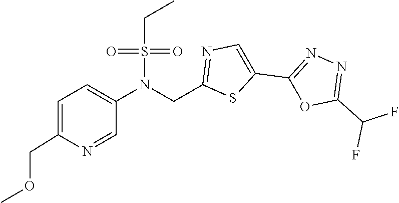 [SMILES: CCS(=O)(=O)N(Cc1ncc(-c2nnc(C(F)F)o2)s1)c1ccc(COC)nc1] | 1H NMR (400 MHz, CDCl3) δ 8.63 (d, J = 2.4 Hz, 1 H), 8.38 (s, 1 H), 7.83 (dd, J = 2.4, 8.0 Hz, 1 H), 7.48 (d, J = 8.4 Hz, 1 H), 6.91 (t, J = 51.6 Hz, 1 H), 5.29 (s, 2 H), 4.57 (s, 2 H), 3.49 (s, 3 H), 3.20 (q, J = 7.6 Hz, 2 H), 1.46 (t, J = 7.6 Hz, 3 H). LCMS RT = 0.789 min, m/z = 446.2 | 0.051 |
| | N-((5-(5-(difluoromethyl)-1,3,4- | | |
| | oxadiazol-2-yl)thiazol-2- | | |
| | yl)methyl)-N-(6- | | |
| | (methoxymethyl)pyridin-3- | | |
| | yl)ethanesulfonamide | | |
| I-105 | 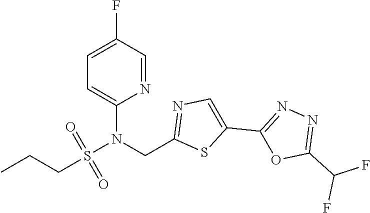 [SMILES: CCCS(=O)(=O)N(Cc1ncc(-c2nnc(C(F)F)o2)s1)c1ccc(F)cn1] | ¹H NMR (400 MHz, CDCl₃) δ 8.37 (s, 1 H), 8.28 (d, J = 3.2 Hz, 1 H), 7.69-7.63 (m, 1 H), 7.51-7.43 (m, 1 H), 6.89 (t, J = 51.6 Hz, 1 H), 5.52 (s, 2 H), 3.20-3.14 (m, 2 H), 1.91- 1.83 (m, 2 H), 1.05 (t, J = 7.6 Hz, 3 H). LCMS RT = 0.684 min, m/z = 433.9 | 0.0268 |
| | N-({5-[5-(difluoromethyl)-1,3,4- | | |
| | oxadiazol-2-yl]-1,3-thiazol-2- | | |
| | yl}methyl)-N-(5-fluoropyridin-2- | | |
| | yl)propane-1-sulfonamide | | |
| I-106 | 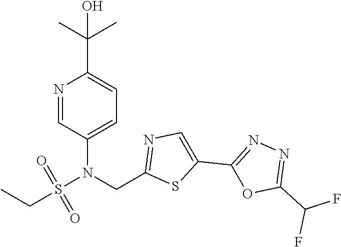 [SMILES: CCS(=O)(=O)N(Cc1ncc(-c2nnc(C(F)F)o2)s1)c1ccc(C(C)(C)O)nc1] | ¹H NMR (400 MHz, CDCl₃) δ 8.55 (s, 1 H), 8.38 (s, 1 H), 7.85 (d, J = 8.8 Hz, 1 H), 7.60 (d, J = 8.4 Hz, 1 H), 6.89 (t, J = 52.0 Hz, 1 H), 5.58 (s, 2 H), 3.30 (q, J = 7.2 Hz, 2 H), 2.63 (s, 1 H), 1.39 (t, J = 7.6 Hz, 3 H), 1.30-1.23 (m, 6 H). LCMS RT = 1.467 min, m/z = 460.2 | 0.480 |
| | N-({5-[5-(difluoromethyl)-1,3,4- | | |
| | oxadiazol-2-yl]-1,3-thiazol-2- | | |
| | yl}methyl)-N-[6-(2- | | |
| | hydroxypropan-2-yl)pyridin-3- | | |
| | yl]ethane-1-sulfonamide | | |
| I-107 | 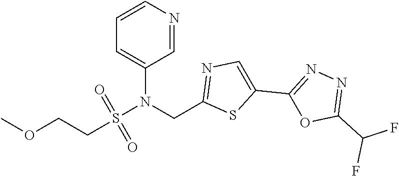 [SMILES: COCCS(=O)(=O)N(Cc1ncc(-c2nnc(C(F)F)o2)s1)c1cccnc1] | ¹H NMR (400 MHz, CDCl₃) δ 8.80 (s, 1 H), 8.57 (d, J = 3.6 Hz, 1 H), 8.34 (s, 1 H), 7.91- 7.88 (m, 1 H), 7.36-7.33 (m, 1 H), 6.90 (t, J = 51.6 Hz, 1 H), 5.24 (s, 2 H), 3.87 (t, J = 5.2 Hz, 2 H), 3.51 (s, 3 H), 3.35 (t, J = 5.2 Hz, 2 H). LCMS RT = 1.284 min, m/z = 431.9 | 0.0212 |
| | N-({5-[5-(difluoromethyl)-1,3,4- | | |
| | oxadiazol-2-yl]-1,3-thiazol-2- | | |
| | yl}methyl)-2-methoxy-N-(pyridin- | | |
| | 3-yl)ethane-1-sulfonamide | | |
| I-108 | 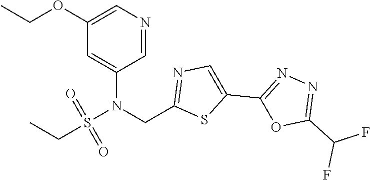 [SMILES: CCOc1cncc(N(Cc2ncc(-c3nnc(C(F)F)o3)s2)S(=O)(=O)CC)c1] | ¹H NMR (400 MHz, CDCl₃) δ 8.40 (s, 1 H), 8.32-8.27 (m, 2 H), 7.42-7.38 (m, 1 H), 6.92 (t, J = 51.6 Hz, 1 H), 5.31 (s, 2 H), 4.10 (q, J = 6.8 Hz, 2 H), 3.22 (q, J = 7.2 Hz, 2 H), 1.49- 1.43 (m, 6 H). LCMS RT = 0.604 min, m/z = 446.0 | 0.0138 |
| | N-({5-[5-(difluoromethyl)-1,3,4- | | |
| | oxadiazol-2-yl]-1,3-thiazol-2- | | |
| | yl}methyl)-N-(5-ethoxypyridin-3- | | |
| | yl)ethane-1-sulfonamide | | |
| I-109 | 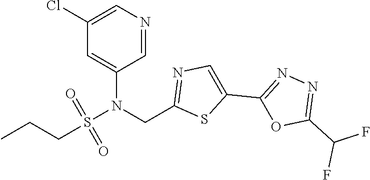 [SMILES: CCCS(=O)(=O)N(Cc1ncc(-c2nnc(C(F)F)o2)s1)c1cncc(Cl)c1] | ¹H NMR (400 MHz, CDCl₃) δ 8.60 (d, J = 2.0 Hz, 1 H), 8.54 (s, 1 H), 8.40 (s, 1 H), 7.88 (t, J = 2.4 Hz, 1 H), 6.91 (t, J = 51.6 Hz, 1 H), 5.27 (s, 2 H), 3.22-3.00 (m, 2 H), 1.99- 1.70 (m, 2 H), 1.09 (t, J = 7.6 Hz, 3 H). LCMS RT = 0.668 min, m/z = 449.9 | 0.00392 |
| | N-(5-chloropyridin-3-yl)-N-({5- | | |
| | [5-(difluoromethyl)-1,3,4- | | |
| | oxadiazol-2-yl]-1,3-thiazol-2- | | |
| | yl}methyl)propane-1-sulfonamide | | |
| I-110 | 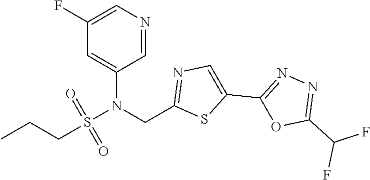 [SMILES: CCCS(=O)(=O)N(Cc1ncc(-c2nnc(C(F)F)o2)s1)c1cncc(F)c1] | ¹H NMR (400 MHz, CDCl₃) δ 8.56 (s, 1 H), 8.46 (d, J = 2.4 Hz, 1 H), 8.40 (s, 1 H), 7.67- 7.61 (m, 1 H), 6.91 (t, J = 51.6 Hz, 1 H), 5.29 (s, 2 H), 3.19- 3.09 (m, 2 H), 1.97-1.85 (m, 2 H), 1.08 (t, J = 7.2 Hz, 3 H). LCMS RT = 2.144 min, m/z = 434.2 | 0.0104 |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)propane-1-sulfonamide | | |
| I-111 | 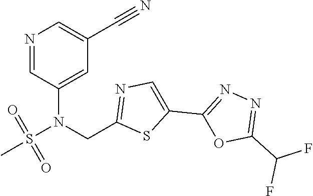 [SMILES: CS(=O)(=O)N(Cc1ncc(-c2nnc(C(F)F)o2)s1)c1cncc(C#N)c1] | ¹H NMR (400 MHz, CDCl₃) δ 8.98 (d, J = 2.4 Hz, 1 H), 8.84 (s, 1 H), 8.45 (s, 1 H), 8.20 (s, 1 H), 6.93 (t, J = 51.2 Hz, 1 H), 5.29 (s, 2 H), 3.11 (s, 3 H). LCMS RT = 1.456 min, m/z = 412.8 | 0.0374 |
| | N-(5-cyanopyridin-3-yl)-N-({5-[5- | | |
| | (difluoromethyl)-1,3,4-oxadiazol- | | |
| | 2-yl]-1,3-thiazol-2- | | |
| | yl}methyl)methanesulfonamide | | |
| I-112 | 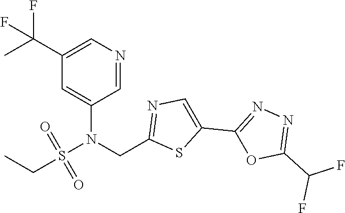 [SMILES: CCS(=O)(=O)N(Cc1ncc(-c2nnc(C(F)F)o2)s1)c1cncc(C(C)(F)F)c1] | ¹H NMR (400 MHz, CDCl₃) δ 8.79 (s, 1 H), 8.72 (s, 1 H), 8.40 (s, 1 H), 7.96 (s, 1 H), 6.91 (t, J = 52.0 Hz, 1 H), 5.30 (s, 2 H), 3.21 (q, J = 7.2 Hz, 2 H), 1.97 (t, J = 18.4 Hz, 3 H), 1.46 (t, J = 7.6 Hz, 3 H). LCMS RT = 1.138 min, m/z = 466.2 | 0.0101 |
| | N-[5-(1,1-difluoroethyl)pyridin-3- | | |
| | yl]-N-({5-[5-(difluoromethyl)- | | |
| | 1,3,4-oxadiazol-2-yl]-1,3-thiazol- | | |
| | 2-yl}methyl)ethane-1-sulfonamide | | |
| I-113 | 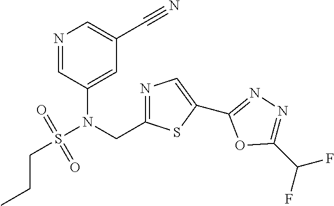 [SMILES: CCCS(=O)(=O)N(Cc1ncc(-c2nnc(C(F)F)o2)s1)c1cncc(C#N)c1] | ¹H NMR (400 MHz, CDCl₃) δ 8.95 (d, J = 2.4 Hz, 1 H), 8.82 (d, J = 2.0 Hz, 1 H), 8.42 (s, 1 H), 8.20-8.12 (m, 1 H), 6.92 (t, J = 51.6 Hz, 1 H), 5.29 (s, 2 H), 3.19-3.08 (m, 2 H), 1.98- 1.85 (m, 2 H), 1.09 (t, J = 7.6 Hz, 3 H). LCMS RT = 1.560 min, m/z = 441.2 | 0.0429 |
| | N-(5-cyanopyridin-3-yl)-N-({5-[5- | | |
| | (difluoromethyl)-1,3,4-oxadiazol- | | |
| | 2-yl]-1,3-thiazol-2- | | |
| | yl}methyl)propane-1-sulfonamide | | |
| I-114 | 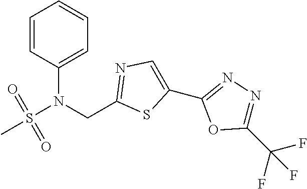 [SMILES: CS(=O)(=O)N(Cc1ncc(-c2nnc(C(F)(F)F)o2)s1)c1ccccc1] | ¹H NMR (400 MHz, CDCl₃) δ 8.39 (s, 1 H), 7.48-7.38 (m, 5 H), 5.28 (s, 2 H), 3.06 (s, 3 H). LCMS RT = 2.568 min, m/z = 404.8 | 0.354 |
| | N-phenyl-N-({5-[5- | | |
| | (trifluoromethyl)-1,3,4-oxadiazol- | | |
| | 2-yl]-1,3-thiazol-2- | | |
| | yl}methyl)methanesulfonamide | | |
| I-115 | 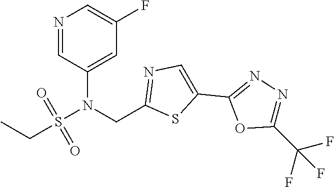 [SMILES: CCS(=O)(=O)N(Cc1ncc(-c2nnc(C(F)(F)F)o2)s1)c1cncc(F)c1] | ¹H NMR (400 MHz, CDCl₃) δ 8.57 (s, 1 H), 8.47 (d, J = 2.4 Hz, 1 H), 8.42 (s, 1 H), 7.68- 7.63 (m, 1 H), 5.31 (s, 2 H), 3.22 (q, J = 7.2 Hz, 2 H), 1.46 (t, J = 7.2 Hz, 3 H). LCMS RT = 0.904 min, m/z = 437.9 | 0.022 |
| | N-(5-fluoropyridin-3-yl)-N-({5-[5- | | |
| | (trifluoromethyl)-1,3,4-oxadiazol- | | |
| | 2-yl]-1,3-thiazol-2- | | |
| | yl}methyl)ethane-1-sulfonamide | | |
| I-116 | 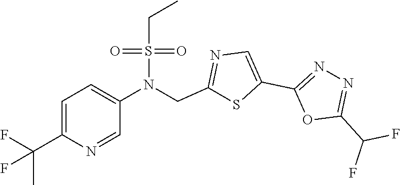 [SMILES: CCS(=O)(=O)N(Cc1ncc(-c2nnc(C(F)F)o2)s1)c1ccc(C(C)(F)F)nc1] | ¹H NMR (400 MHz, CDCl₃) δ 8.74 (d, J = 2.4 Hz, 1 H), 8.39 (s, 1 H), 7.98-7.92 (m, 1 H), 7.70 (d, J = 8.4 Hz, 1 H), 6.91 (t, J = 51.6 Hz, 1 H), 5.32 (s, 2 H), 3.22 (q, J = 7.6 Hz, 2 H), 2.01 (t, J = 18.8 Hz, 3 H), 1.46 (t, J = 7.6 Hz, 3 H). LCMS RT = 0.908 min, m/z = 465.9 | 0.0514 |
| | N-[6-(1,1-difluoroethyl)pyridin-3- | | |
| | yl]-N-({5-[5-(difluoromethyl)- | | |
| | 1,3,4-oxadiazol-2-yl]-1,3-thiazol- | | |
| | 2-yl}methyl)ethane-1-sulfonamide | | |
| I-117 | 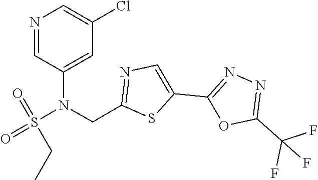 [SMILES: CCS(=O)(=O)N(Cc1ncc(-c2nnc(C(F)(F)F)o2)s1)c1cncc(Cl)c1] | ¹H NMR (400 MHz, CDCl₃) δ 8.61 (d, J = 2.4 Hz, 1 H), 8.55 (d, J = 2.4 Hz, 1 H), 8.42 (s, 1 H), 7.88 (t, J = 2.0 Hz, 1 H), 5.29 (s, 2 H), 3.21 (q, J = 4.0 Hz, 2 H), 1.46 (t, J = 8.0 Hz, 3 H). LCMS RT = 2.591 min, m/z = 453.8 | 0.0245 |
| | N-(5-chloropyridin-3-yl)-N-({5- | | |
| | [5-(trifluoromethyl)-1,3,4- | | |
| | oxadiazol-2-yl]-1,3-thiazol-2- | | |
| | yl}methyl)ethane-1-sulfonamide | | |
| I-118 | 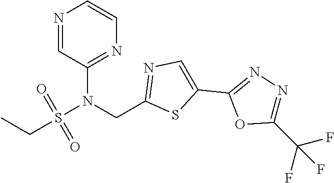 [SMILES: CCS(=O)(=O)N(Cc1ncc(-c2nnc(C(F)(F)F)o2)s1)c1cnccn1] | ¹H NMR (400 MHz, CDCl₃) δ 9.00 (s, 1 H), 8.44-8.37 (m, 3 H), 5.58 (s, 2 H), 3.41 (d, J = 6.8 Hz, 2 H), 1.43 (t, J = 6.8 Hz, 3 H). LCMS RT = 0.895 min, m/z = 420.9 | 0.0278 |
| | N-(pyrazin-2-yl)-N-({5-[5- | | |
| | (trifluoromethyl)-1,3,4-oxadiazol- | | |
| | 2-yl]-1,3-thiazol-2- | | |
| | yl}methyl)ethane-1-sulfonamide | | |
| I-119 | 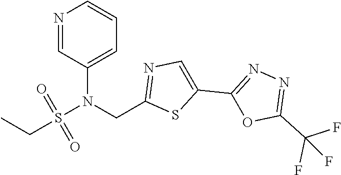 [SMILES: CCS(=O)(=O)N(Cc1ncc(-c2nnc(C(F)(F)F)o2)s1)c1cccnc1] | ¹H NMR (400 MHz, CDCl₃) δ 8.73 (s, 1 H), 8.62-8.60 (m, 1 H), 8.41 (s, 1 H), 7.86 (d, J = 8.0 Hz, 1 H), 7.40-7.35 (m, 1 H), 5.32 (s, 2 H), 3.22 (q, J = 6.8 Hz, 2 H), 1.48 (t, J = 7.2 Hz, 3 H). LCMS RT = 1.897 min, m/z = 420.1 | 0.042 |
| | N-(pyridin-3-yl)-N-({5-[5- | | |
| | (trifluoromethyl)-1,3,4-oxadiazol- | | |
| | 2-yl]-1,3-thiazol-2- | | |
| | yl}methyl)ethane-1-sulfonamide | | |
| I-120 | 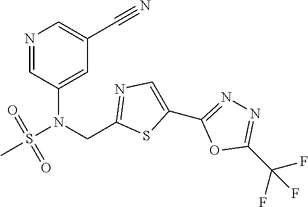 [SMILES: CS(=O)(=O)N(Cc1ncc(-c2nnc(C(F)(F)F)o2)s1)c1cncc(C#N)c1] | ¹H NMR (400 MHz, CDCl₃) δ 8.98 (d, J = 2.8 Hz, 1 H), 8.85 (s, 1 H), 8.46 (s, 1 H), 8.20 (t, J = 2.0 Hz, 1 H), 5.30 (s, 2 H), 3.11 (s, 3 H). LCMS RT = 2.105 min, m/z = 431.1 | 0.111 |
| | N-(5-cyanopyridin-3-yl)-N-({5-[5- | | |
| | (trifluoromethyl)-1,3,4-oxadiazol- | | |
| | 2-yl]-1,3-thiazol-2- | | |
| | yl}methyl)methanesulfonamide | | |
| I-121 | 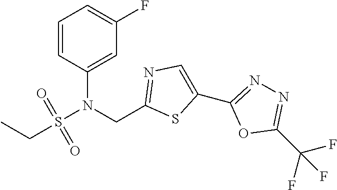 [SMILES: CCS(=O)(=O)N(Cc1ncc(-c2nnc(C(F)(F)F)o2)s1)c1cccc(F)c1] | ¹H NMR (400 MHz, CDCl₃) δ 8.40 (s, 1 H), 7.41-7.36 (m, 1 H), 7.28 (s, 1 H), 7.25-7.22 (m, 1 H), 7.09-7.04 (m, 1 H), 5.30 (s, 2 H), 3.19 (q, J = 7.6 Hz, 2 H), 1.45 (t, J = 7.2 Hz, 3 H). LCMS RT = 1.978 min, m/z = 437.1 | 0.0839 |
| | N-(3-fluorophenyl)-N-({5-[5- | | |
| | (trifluoromethyl)-1,3,4-oxadiazol- | | |
| | 2-yl]-1,3-thiazol-2- | | |
| | yl}methyl)ethane-1-sulfonamide | | |
| I-122 | 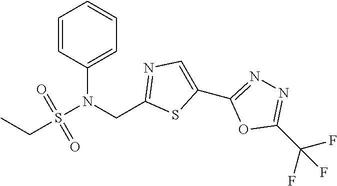 [SMILES: CCS(=O)(=O)N(Cc1ncc(-c2nnc(C(F)(F)F)o2)s1)c1ccccc1] | ¹H NMR (400 MHz, CDCl₃) δ 8.38 (s, 1 H), 7.50-7.30 (m, 5 H), 5.31 (s, 2 H), 3.17 (q, J = 7.2 Hz, 2 H), 1.45 (t, J = 7.2 Hz, 3 H). LCMS RT = 0.973 min, m/z = 419.0 | 0.0249 |
| | N-phenyl-N-({5-[5- | | |
| | (trifluoromethyl)-1,3,4-oxadiazol- | | |
| | 2-yl]-1,3-thiazol-2- | | |
| | yl}methyl)ethane-1-sulfonamide | | |
| I-123 | 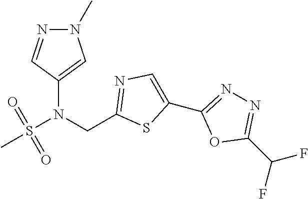 [SMILES: Cn1cc(N(Cc2ncc(-c3nnc(C(F)F)o3)s2)S(C)(=O)=O)cn1] | ¹H NMR (400 MHz, CDCl₃) δ 8.42 (s, 1 H), 7.57 (s, 1 H), 7.48 (s, 1 H), 6.91 (t, J = 51.6 Hz, 1 H), 5.14 (s, 2 H), 3.88 (s, 3 H), 3.03 (s, 3 H). LCMS RT = 1.057 min, m/z = 390.9 | 0.0388 |
| | N-({5-[5-(difluoromethyl)-1,3,4-oxadiazol-2-yl]-1,3-thiazol-2- | | |
| | yl}methyl)-N-(1-methyl-1H- | | |
| | pyrazol-4-yl)methanesulfonamide | | |
| I-124 | 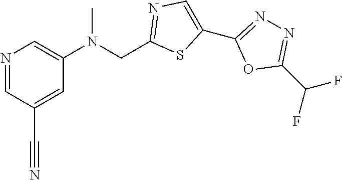 [SMILES: CN(Cc1ncc(-c2nnc(C(F)F)o2)s1)c1cncc(C#N)c1] | ¹H NMR (400 MHz, CDCl₃) δ 8.46 (s, 1 H), 8.41 (s, 1 H), 8.33 (s, 1 H), 7.25 (s, 1 H), 6.90 (t, J = 51.6 Hz, 1 H), 4.92 (s, 2 H), 3.27 (s, 3 H). LCMS RT = 0.971 min, m/z = 349.0 | 0.0636 |
| | 5-[({5-[5-(difluoromethyl)-1,3,4- | | |
| | oxadiazol-2-yl]-1,3-thiazol-2- | | |
| | yl}methyl)(methyl)amino]pyridine- | | |
| | 3-carbonitrile | | |
| I-125 | 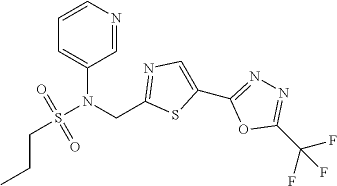 [SMILES: CCCS(=O)(=O)N(Cc1ncc(-c2nnc(C(F)(F)F)o2)s1)c1cccnc1] | 1H NMR (400 MHz, CDCl3) δ 8.71 (d, J = 2.4 Hz, 1 H), 8.60- 8.56 (m, 1 H), 8.38 (s, 1 H), 7.85-7.82 (m, 1 H), 7.39- 7.32 (m, 1 H), 6.91 (t, J = 51.6 Hz, 1 H), 5.29 (s, 2 H), 3.18- 3.11 (m, 2 H), 1.98-1.89 (m, 2 H), 1.09 (t, J = 7.2 Hz, 3 H). LCMS RT = 0.776 min, m/z = 416.1 | 0.0599 |
| | N-({5-[5-(difluoromethyl)-1,3,4- | | |
| | oxadiazol-2-yl]-1,3-thiazol-2- | | |
| | yl}methyl)-N-(pyridin-3- | | |
| | yl)propane-1-sulfonamide | | |
| I-126 | 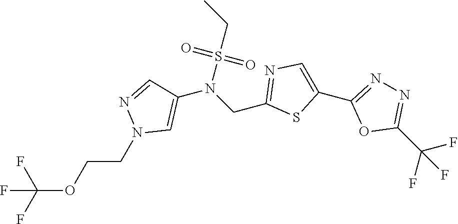 [SMILES: CCS(=O)(=O)N(Cc1ncc(-c2nnc(C(F)(F)F)o2)s1)c1cnn(CCOC(F)(F)F)c1] | ¹H NMR (400 MHz, CDCl₃) δ 8.39 (s, 1 H), 7.64 (s, 1 H), 7.57 (s, 1 H), 6.91 (t, J = 51.6 Hz, 1 H), 5.16 (s, 2 H), 4.36- 4.33 (m, 2 H), 4.30-4.23 (m, 2 H), 3.15 (q, J = 7.6 Hz, 2 H), 1.40 (t, J = 7.2 Hz, 3 H). LCMS RT = 1.043 min, m/z = 502.9 | 0.134 |
| | N-({5-[5-(difluoromethyl)-1,3,4- | | |
| | oxadiazol-2-yl]-1,3-thiazol-2- | | |
| | yl}methyl)-N-{1-[2- | | |
| | (trifluoromethoxy)ethyl]-1H- | | |
| | pyrazol-4-yl}ethane-1- | | |
| | sulfonamide | | |
| I-127 | 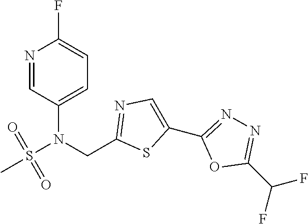 [SMILES: CS(=O)(=O)N(Cc1ncc(-c2nnc(C(F)F)o2)s1)c1ccc(F)nc1] | ¹H NMR (400 MHz, CDCl₃) δ 8.37 (s, 1 H), 8.29 (d, J = 3.2 Hz, 1 H), 7.67-7.61 (m, 1 H), 7.53-7.46 (m, 1 H), 6.90 (t, J = 51.6 Hz, 1 H), 5.50 (s, 2 H), 3.10 (s, 3 H). LCMS RT = 0.854 min, m/z = 406.1 | 0.0215 |
| | N-({5-[5-(difluoromethyl)-1,3,4-oxadiazol-2-yl]-1,3-thiazol-2- | | |
| | yl}methyl)-N-(5-fluoropyridin-2- | | |
| | yl)methanesulfonamide | | |
| I-128 | 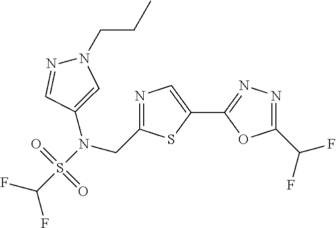 [SMILES: CCCn1cc(N(Cc2ncc(-c3nnc(C(F)F)o3)s2)S(=O)(=O)C(F)F)cn1] | ¹H NMR (400 MHz, CDCl₃) δ 8.42 (s, 1 H), 7.49 (d, J = 4.4 Hz, 2 H), 6.92 (t, J = 51.6 Hz, 1 H), 6.44 (t, J = 53.2 Hz, 1 H), 5.21 (s, 2 H), 4.02 (t, J = 7.2 Hz, 2 H), 1.94-1.78 (m, 2 H), 0.88 (t, J = 7.6 Hz, 3 H). LCMS RT = 0.901 min, m/z = 455.1 | 0.251 |
| | N-({5-[5-(difluoromethyl)-1,3,4- | | |
| | oxadiazol-2-yl]-1,3-thiazol-2- | | |
| | yl}methyl)-1,1-difluoro-N-(1- | | |
| | propyl-1H-pyrazol-4- | | |
| | yl)methanesulfonamide | | |
| I-129 | 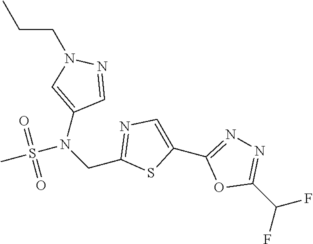 [SMILES: CCCn1cc(N(Cc2ncc(-c3nnc(C(F)F)o3)s2)S(C)(=O)=O)cn1] | ¹H NMR (400 MHz, CDCl₃) δ 8.41 (s, 1 H), 7.58 (s, 1 H), 7.49 (s, 1 H), 6.91 (t, J = 51.6 Hz, 1 H), 5.14 (s, 2 H), 4.03 (t, J = 7.2 Hz, 2 H), 3.03 (s, 3 H), 1.92-1.81 (m, 2 H), 0.90 (t, J = 7.2 Hz, 3 H). LCMS RT = 0.795 min, m/z = 418.9 | 0.118 |
| | N-({5-[5-(difluoromethyl)-1,3,4- | | |
| | oxadiazol-2-yl]-1,3-thiazol-2- | | |
| | yl}methyl)-N-(1-propyl-1H- | | |
| | pyrazol-4-yl)methanesulfonamide | | |
| I-130 | 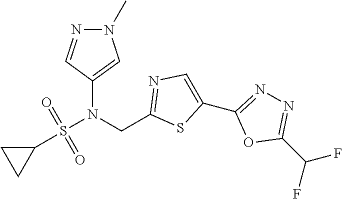 [SMILES: Cn1cc(N(Cc2ncc(-c3nnc(C(F)F)o3)s2)S(=O)(=O)C2CC2)cn1] | ¹H NMR (400 MHz, CDCl₃) δ 8.39 (s, 1 H), 7.56 (s, 1 H), 7.52 (s, 1 H), 6.91 (t, J = 51.6 Hz, 1 H), 5.15 (s, 2 H), 3.87 (s, 3 H), 2.58-2.50 (m, 1 H), 1.20-1.15 (m, 2 H), 1.07- 1.01 (m, 2 H). LCMS RT = 1.320 min, m/z = 416.8 | 0.0348 |
| | N-({5-[5-(difluoromethyl)-1,3,4- | | |
| | oxadiazol-2-yl]-1,3-thiazol-2- | | |
| | yl}methyl)-N-(1-methyl-1H- | | |
| | pyrazol-4- | | |
| | yl)cyclopropanesulfonamide | | |
| I-131 | 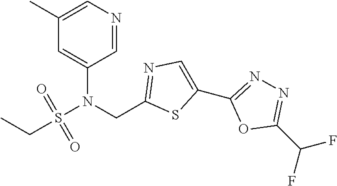 [SMILES: CCS(=O)(=O)N(Cc1ncc(-c2nnc(C(F)F)o2)s1)c1cncc(C)c1] | ¹H NMR (400 MHz, CDCl₃) δ 8.50 (d, J = 2.4 Hz, 1 H), 8.41 (s, 1 H), 8.38 (s, 1 H), 7.65 (s, 1 H), 6.91 (t, J = 51.6 Hz, 1 H), 5.29 (s, 2 H), 3.20 (q, J = 7.6 Hz, 2 H), 2.37 (s, 3 H), 1.47 (t, J = 7.6 Hz, 3 H). LCMS RT = 0.714 min, m/z = 416.2 | 0.013 |
| | N-({5-[5-(difluoromethyl)-1,3,4- | | |
| | oxadiazol-2-yl]-1,3-thiazol-2- | | |
| | yl}methyl)-N-(5-methylpyridin-3- | | |
| | yl)ethane-1-sulfonamide | | |
| I-132 | 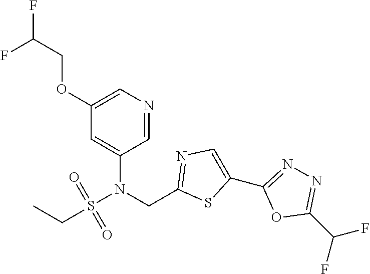 [SMILES: CCS(=O)(=O)N(Cc1ncc(-c2nnc(C(F)F)o2)s1)c1cncc(OCC(F)F)c1] | ¹H NMR (400 MHz, CDCl₃) δ 8.40 (s, 2 H), 8.32 (d, J = 2.4 Hz, 1 H), 7.45 (t, J = 2.4 Hz, 1 H), 6.91 (t, J = 51.6 Hz, 1 H), 6.25-5.96 (m, 1 H), 5.30 (s, 2 H), 4.30-4.20 (m, 2 H), 3.21 (q, J = 7.2 Hz, 2 H), 1.46 (t, J = 7.6 Hz, 3 H). LCMS RT = 0.857 min, m/z = 482.2 | 0.00964 |
| | N-[5-(2,2-difluoroethoxy)pyridin- | | |
| | 3-yl]-N-({5-[5-(difluoromethyl)- | | |
| | 1,3,4-oxadiazol-2-yl]-1,3-thiazol- | | |
| | 2-yl}methyl)ethane-1-sulfonamide | | |
| I-133 | 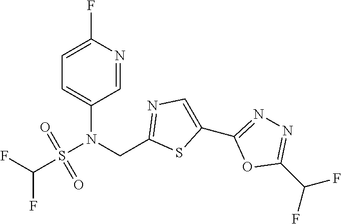 [SMILES: O=S(=O)(C(F)F)N(Cc1ncc(-c2nnc(C(F)F)o2)s1)c1ccc(F)nc1] | ¹H NMR (400 MHz, CDCl₃) δ 8.39 (s, 1 H), 8.30 (d, J = 2.8 Hz, 1 H), 7.58-7.55 (m, 1 H), 7.54-7.48 (m, 1 H), 7.03 (t, J = 51.6 Hz, 1 H), 6.33 (t, J = 53.6 Hz, 1 H), 5.55 (s, 2 H). LCMS RT = 0.954 min, m/z = 441.9 | 0.0857 |
| | N-({5-[5-(difluoromethyl)-1,3,4- | | |
| | oxadiazol-2-yl]-1,3-thiazol-2- | | |
| | yl}methyl)-1,1-difluoro-N-(5- | | |
| | fluoropyridin-2- | | |
| | yl)methanesulfonamide | | |
| I-134 | 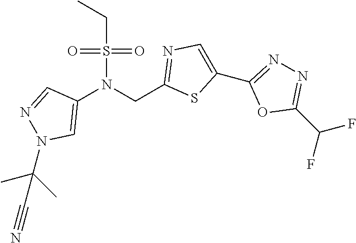 [SMILES: CCS(=O)(=O)N(Cc1ncc(-c2nnc(C(F)F)o2)s1)c1cnn(C(C)(C)C#N)c1] | ¹H NMR (400 MHz, CDCl₃) δ 8.42 (s, 1 H), 7.83 (s, 1 H), 7.63 (s, 1 H), 6.91 (t, J = 51.6 Hz, 1 H), 5.17 (s, 2 H), 3.17 (q, J = 7.2 Hz, 2 H), 1.99 (s, 6 H), 1.42 (t, J = 7.2 Hz, 3 H). LCMS RT = 1.012 min, m/z = 458.1 | 0.121 |
| | N-[1-(1-cyano-1-methylethyl)-1H- | | |
| | pyrazol-4-yl]-N-({5-[5- | | |
| | (difluoromethyl)-1,3,4-oxadiazol- | | |
| | 2-yl]-1,3-thiazol-2- | | |
| | yl}methyl)ethane-1-sulfonamide | | |
| I-135 | 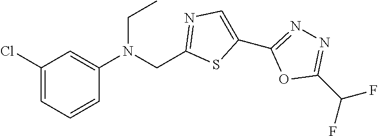 [SMILES: CCN(Cc1ncc(-c2nnc(C(F)F)o2)s1)c1cccc(Cl)c1] | ¹H NMR (400 MHz, CDCl₃) δ 8.45 (s, 1 H), 7.14 (t, J = 8.0 Hz, 1 H), 6.88 (t, J = 51.6 Hz, 1 H), 6.76-6.74 (m, 2 H), 6.64-6.60 (m, 1 H), 4.79 (s, 2 H), 3.58 (q, J = 7.2 Hz, 2 H), 1.30 (t, J = 7.2 Hz, 3 H). LCMS RT = 1.098 min, m/z = 370.9 | 0.0531 |
| | 3-chloro-N-({5-[5- | | |
| | (difluoromethyl)-1,3,4-oxadiazol- | | |
| | 2-yl]-1,3-thiazol-2-yl}methyl)-N- | | |
| | ethylaniline | | |
| I-136 | 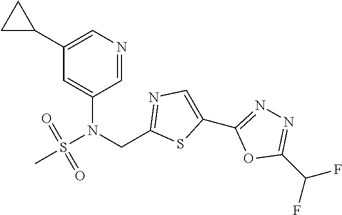 [SMILES: CS(=O)(=O)N(Cc1ncc(-c2nnc(C(F)F)o2)s1)c1cncc(C2CC2)c1] | ¹H NMR (400 MHz, CDCl₃) δ 8.46 (s, 1 H), 8.41 (s, 1 H), 8.37 (s, 1 H), 7.46 (s, 1 H), 6.91 (t, J = 51.6 Hz, 1 H), 5.25 (s, 2 H), 3.09 (s, 3 H), 1.96- 1.89 (m, 1 H), 1.14-1.06 (m, 2 H), 0.79-0.71 (m, 2 H). LCMS RT = 0.743 min, m/z = 427.9 | 0.013 |
| | N-(5-cyclopropylpyridin-3-yl)-N- | | |
| | ({5-[5-(difluoromethyl)-1,3,4- | | |
| | oxadiazol-2-yl]-1,3-thiazol-2- | | |
| | yl}methyl)methanesulfonamide | | |
| I-137 | 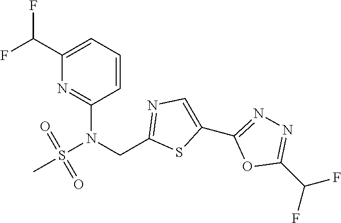 [SMILES: CS(=O)(=O)N(Cc1ncc(-c2nnc(C(F)F)o2)s1)c1cccc(C(F)F)n1] | ¹H NMR (400 MHz, CDCl₃) δ 8.38 (s, 1 H), 7.90 (t, J = 8.0 Hz, 1 H), 7.77 (d, J = 8.4 Hz, 1 H), 7.48 (d, J = 7.6 Hz, 1 H), 6.89 (t, J = 51.6 Hz, 1 H), 6.54 (t, J = 55.2 Hz, 1 H), 5.59 (s, 2 H), 3.23 (s, 3 H). LCMS RT = 0.899 min, m/z = 437.9 | 0.0107 |
| | N-({5-[5-(difluoromethyl)-1,3,4- | | |
| | oxadiazol-2-yl]-1,3-thiazol-2- | | |
| | yl}methyl)-N-[6- | | |
| | (difluoromethyl)pyridin-2- | | |
| | yl]methanesulfonamide | | |
| I-138 | 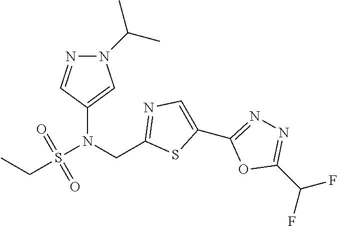 [SMILES: CCS(=O)(=O)N(Cc1ncc(-c2nnc(C(F)F)o2)s1)c1cnn(C(C)C)c1] | ¹H NMR (400 MHz, CDCl₃) δ 8.37 (s, 1 H), 7.57 (s, 1 H), 7.46 (s, 1 H), 6.87 (t, J = 51.2 Hz, 1 H), 5.12 (s, 2 H), 4.44- 4.34 (m, 1 H), 3.13 (q, J = 7.2 Hz, 2 H), 1.45 (d, J = 6.8 Hz, 6 H), 1.39 (t, J = 7.2 Hz, 3 H). LCMS RT = 1.141 min, m/z = 433.2 | 0.043 |
| | N-({5-[5-(difluoromethyl)-1,3,4- | | |
| | oxadiazol-2-yl]-1,3-thiazol-2- | | |
| | yl}methyl)-N-[1-(propan-2-yl)- | | |
| | 1H-pyrazol-4-yl]ethane-1- | | |
| | sulfonamide | | |
| I-139 | 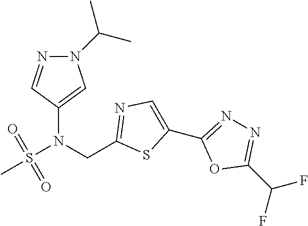 [SMILES: CC(C)n1cc(N(Cc2ncc(-c3nnc(C(F)F)o3)s2)S(C)(=O)=O)cn1] | ¹H NMR (400 MHz, CDCl₃) δ 8.39 (s, 1 H), 7.58 (s, 1 H), 7.46 (s, 1 H), 6.88 (t, J = 51.6 Hz, 1 H), 5.11 (s, 2 H), 4.45- 4.35 (m, 1 H), 3.00 (s, 3 H), 1.46 (d, J = 6.8 Hz, 6 H). LCMS RT = 0.997 min, m/z = 419.2 | 0.118 |
| | N-({5-[5-(difluoromethyl)-1,3,4- | | |
| | oxadiazol-2-yl]-1,3-thiazol-2- | | |
| | yl}methyl)-N-[1-(propan-2-yl)- | | |
| | 1H-pyrazol-4- | | |
| | yl]methanesulfonamide | | |
| I-140 | 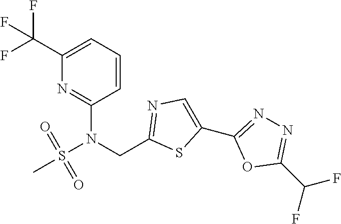 [SMILES: CS(=O)(=O)N(Cc1ncc(-c2nnc(C(F)F)o2)s1)c1cccc(C(F)(F)F)n1] | ¹H NMR (400 MHz, CDCl₃) δ 8.37 (s, 1 H), 7.95-7.85 (m, 2 H), 7.51 (d, J = 7.2 Hz, 1 H), 6.90 (t, J = 51.6 Hz, 1 H), 5.61 (s, 2 H), 3.29 (s, 3 H). LCMS RT = 1.112 min, m/z = 455.8 | 0.0576 |
| | N-({5-[5-(difluoromethyl)-1,3,4- | | |
| | oxadiazol-2-yl]-1,3-thiazol-2- | | |
| | yl}methyl)-N-[6- | | |
| | (trifluoromethyl)pyridin-2- | | |
| | yl]methanesulfonamide | | |
| I-141 | 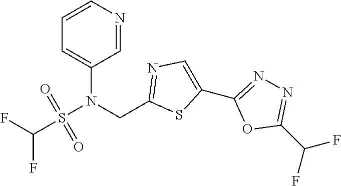 [SMILES: O=S(=O)(C(F)F)N(Cc1ncc(-c2nnc(C(F)F)o2)s1)c1cccnc1] | ¹H NMR (400 MHz, CDCl₃) δ 8.70-8.60 (m, 2 H), 8.39 (s, 1 H), 7.75 (d, J = 6.8 Hz, 1 H), 7.39-7.36 (m, 1 H), 6.92 (t, J = 51.6 Hz, 1 H), 6.49 (t, J = 53.2 Hz, 1 H), 5.35 (s, 2 H). LCMS RT = 0.965 min, m/z = 423.8 | 0.0655 |
| | N-({5-[5-(difluoromethyl)-1,3,4- | | |
| | oxadiazol-2-yl]-1,3-thiazol-2- | | |
| | yl}methyl)-1,1-difluoro-N- | | |
| | (pyridin-3-yl)methanesulfonamide | | |
| I-142 | 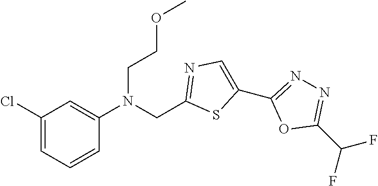 [SMILES: COCCN(Cc1ncc(-c2nnc(C(F)F)o2)s1)c1cccc(Cl)c1] | ¹H NMR (400 MHz, CDCl₃) δ 8.44 (s, 1 H), 7.13 (t, J = 8.0 Hz, 1 H), 6.88 (t, J = 89.2 Hz, 1 H), 6.80-6.70 (m, 2 H), 6.68-6.55 (m, 1H), 4.94 (s, 2 H), 3.74-3.71 (m, 2 H), 3.70- 3.65 (m, 2 H), 3.38 (s, 3 H). LCMS RT = 1.055 min, m/z = 400.9 | 0.0245 |
| | 3-chloro-N-({5-[5- | | |
| | (difluoromethyl)-1,3,4-oxadiazol- | | |
| | 2-yl]-1,3-thiazol-2-yl}methyl)-N- | | |
| | (2-methoxyethyl)aniline | | |
| I-143 | 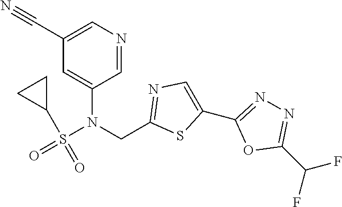 [SMILES: N#Cc1cncc(N(Cc2ncc(-c3nnc(C(F)F)o3)s2)S(=O)(=O)C2CC2)c1] | ¹H NMR (400 MHz, CDCl₃) δ 8.99 (d, J = 2.0 Hz, 1 H), 8.83 (s, 1 H), 8.41 (s, 1 H), 8.18 (t, J = 2.0 Hz, 1 H), 6.92 (t, J = 51.6 Hz, 1H), 5.31 (s, 2 H), 2.59-2.50 (m, 1 H), 1.13- 1.08 (m, 4 H). LCMS RT = 0.834 min, m/z = 439.1 | 0.0153 |
| | N-(5-cyanopyridin-3-yl)-N-({5-[5- | | |
| | (difluoromethyl)-1,3,4-oxadiazol- | | |
| | 2-yl]-1,3-thiazol-2- | | |
| | yl}methyl)cyclopropanesulfonamide | | |
| I-144 | 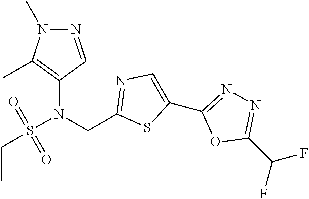 [SMILES: CCS(=O)(=O)N(Cc1ncc(-c2nnc(C(F)F)o2)s1)c1cnn(C)c1C] | ¹H NMR (400 MHz, CDCl₃) δ 8.39 (s, 1 H), 7.38 (s, 1 H), 6.91 (t, J = 51.6 Hz, 1 H), 5.11 (s, 2 H), 3.76 (s, 3 H), 3.20 (q, J = 7.2 Hz, 2 H), 2.25 (s, 3 H), 1.47 (t, J = 7.4 Hz, 3 H). LCMS RT = 0.751 min, m/z = 419.0 | 0.102 |
| | N-({5-[5-(difluoromethyl)-1,3,4- | | |
| | oxadiazol-2-yl]-1,3-thiazol-2- | | |
| | yl}methyl)-N-(1,5-dimethyl-1H- | | |
| | pyrazol-4-yl)ethane-1-sulfonamide | | |
| I-145 | 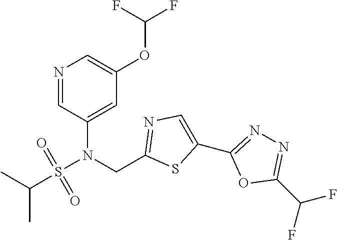 [SMILES: CC(C)S(=O)(=O)N(Cc1ncc(-c2nnc(C(F)F)o2)s1)c1cncc(OC(F)F)c1] | 1H NMR (400 MHz, CDCl3) δ 8.61 (d, J = 2.0 Hz, 1 H), 8.45 (d, J = 2.4 Hz, 1 H), 8.38 (s, 1 H), 7.68 (t, J = 2.0 Hz, 1 H), 6.91 (t, J = 51.6 Hz, 1 H), 6.58 (t, J = 72.0 Hz, 1 H), 5.31 (s, 2 H), 3.40-3.29 (m, 1 H), 1.45 (d, J = 6.8 Hz, 6 H). LCMS RT = 1.625 min, m/z = 482.2 | 0.0102 |
| | N-[5-(difluoromethoxy)pyridin-3- | | |
| | yl]-N-({5-[5-(difluoromethyl)- | | |
| | 1,3,4-oxadiazol-2-yl]-1,3-thiazol- | | |
| | 2-yl}methyl)propane-2- | | |
| | sulfonamide | | |
| I-146 | 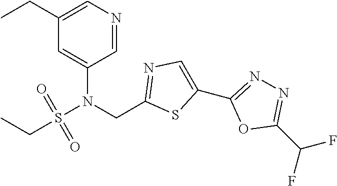 [SMILES: CCc1cncc(N(Cc2ncc(-c3nnc(C(F)F)o3)s2)S(=O)(=O)CC)c1] | 1H NMR (400 MHz, CD3OD) δ 8.59 (s, 1 H), 8.40 (s, 2 H), 8.02 (s, 1 H), 7.19 (t, J = 51.2 Hz, 1 H), 5.40 (s, 2 H), 3.40- 3.30 (m, 2 H), 2.73 (q, J = 7.6 Hz, 2 H), 1.39 (t, J = 7.6 Hz, 3 H), 1.25 (t, J = 7.6 Hz, 3 H). LCMS RT = 1.215 min, m/z = 430.2 | 0.0162 |
| | N-({5-[5-(difluoromethyl)-1,3,4- | | |
| | oxadiazol-2-yl]-1,3-thiazol-2- | | |
| | yl}methyl)-N-(5-ethylpyridin-3- | | |
| | yl)ethane-1-sulfonamide | | |
| I-147 | 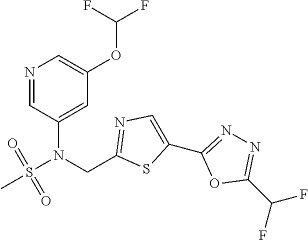 [SMILES: CS(=O)(=O)N(Cc1ncc(-c2nnc(C(F)F)o2)s1)c1cncc(OC(F)F)c1] | 1H NMR (400 MHz, CDCl3) δ 8.62 (s, 1 H), 8.49 (s, 1 H), 8.43 (s, 1 H), 7.69 (s, 1 H), 6.92 (t, J = 51.6 Hz, 1 H), 6.59 (t, J = 72.0 Hz, 1 H), 5.28 (s, 2 H), 3.12 (s, 3 H). LCMS RT = 1.512 min, m/z = 453.8 | 0.019 |
| | N-[5-(difluoromethoxy)pyridin-3-yl]-N-({5-[5-(difluoromethyl)- | | |
| | 1,3,4-oxadiazol-2-yl]-1,3-thiazol- | | |
| | 2-yl}methyl)methanesulfonamide | | |
| I-148 | 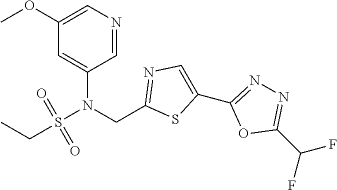 [SMILES: CCS(=O)(=O)N(Cc1ncc(-c2nnc(C(F)F)o2)s1)c1cncc(OC)c1] | 1H NMR (400 MHz, CDCl3) δ ppm 8.39 (s, 1 H), 8.30-8.28 (m, 2 H), 7.38 (s, 1 H), 6.91 (t, J = 51.6 Hz, 1 H), 5.30 (s, 2 H), 3.87 (s, 3 H), 3.21 (q, J = 7.6 Hz, 2 H), 1.46 (t, J = 7.6 Hz, 3 H). LCMS RT = 1.360 min, m/z = 431.9 [M + H]+ | 0.0192 |
| | N-({5-[5-(difluoromethyl)-1,3,4- | | |
| | oxadiazol-2-yl]-1,3-thiazol-2- | | |
| | yl}methyl)-N-(5-methoxypyridin- | | |
| | 3-yl)ethane-1-sulfonamide | | |
| I-149 | 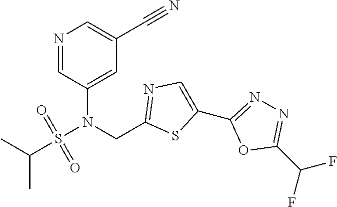 [SMILES: CC(C)S(=O)(=O)N(Cc1ncc(-c2nnc(C(F)F)o2)s1)c1cncc(C#N)c1] | 1H NMR (400 MHz, CDCl3) δ 8.95 (d, J = 2.8 Hz, 1 H), 8.80 (s, 1 H), 8.39 (s, 1 H), 8.16 (s, 1 H), 6.92 (t, J = 51.6 Hz, 1 H), 5.30 (s, 2 H), 3.39-3.25 (m, 1 H), 1.45 (d, J = 6.8 Hz, 6 H). LCMS RT = 1.584 min, m/z = 440.9 | 0.0231 |
| | N-(5-cyanopyridin-3-yl)-N-({5-[5- | | |
| | (difluoromethyl)-1,3,4-oxadiazol- | | |
| | 2-yl]-1,3-thiazol-2- | | |
| | yl}methyl)propane-2-sulfonamide | | |
| I-150 | 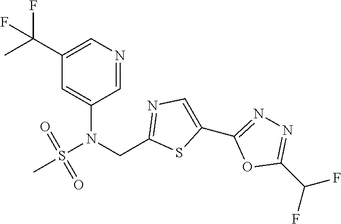 [SMILES: CC(F)(F)c1cncc(N(Cc2ncc(-c3nnc(C(F)F)o3)s2)S(C)(=O)=O)c1] | 1H NMR (400 MHz, CDCl3) δ 8.80 (s, 1 H), 8.75 (s, 1 H), 8.43 (s, 1 H), 7.98 (s, 1 H), 6.92 (t, J = 51.6 Hz, 1 H), 5.29 (s, 2 H), 3.12 (s, 3 H), 1.98 (t, J = 18.0 Hz, 3 H). LCMS RT = 1.554 min, m/z = 451.8 | 0.0255 |
| | N-[5-(1,1-difluoroethyl)pyridin-3- | | |
| | yl]-N-({5-[5-(difluoromethyl)- | | |
| | 1,3,4-oxadiazol-2-yl]-1,3-thiazol- | | |
| | 2-yl}methyl)methanesulfonamide | | |
| I-151 | 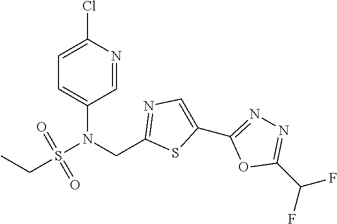 [SMILES: CCS(=O)(=O)N(Cc1ncc(-c2nnc(C(F)F)o2)s1)c1ccc(Cl)nc1] | 1H NMR (400 MHz, CDCl3) δ 8.37 (d, J = 4.0 Hz, 2 H), 7.74- 7.67 (m, 2 H), 6.90 (t, J = 51.6 Hz, 1 H), 5.56 (s, 2 H), 3.29 (q, J = 7.2 Hz, 2 H), 1.39 (t, J = 7.2 Hz, 3 H). LCMS RT = 1.826 min, m/z = 436.1 | 0.0315 |
| | N-(5-chloropyridin-2-yl)-N-({5- | | |
| | [5-(difluoromethyl)-1,3,4- | | |
| | oxadiazol-2-yl]-1,3-thiazol-2- | | |
| | yl}methyl)ethane-1-sulfonamide | | |
| I-152 | 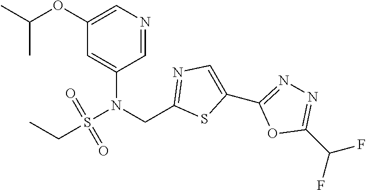 [SMILES: CCS(=O)(=O)N(Cc1ncc(-c2nnc(C(F)F)o2)s1)c1cncc(OC(C)C)c1] | 1H NMR (400 MHz, CDCl3) δ 8.39 (s, 1 H), 8.25 (s, 2 H), 7.35 (s, 1 H), 6.91 (t, J = 51.6 Hz, 1 H), 5.29 (s, 2 H), 4.61- 4.55 (m, 1 H), 3.25-3.18 (m, 2 H), 1.61 (s, 3 H), 1.36 (d, J = 4.4 Hz, 6 H). LCMS RT = 1.666 min, m/z = 459.9 | 0.0429 |
| | N-({5-[5-(difluoromethyl)-1,3,4- | | |
| | oxadiazol-2-yl]-1,3-thiazol-2- | | |
| | yl}methyl)-N-[5-(propan-2- | | |
| | yloxy)pyridin-3-yl]ethane-1- | | |
| | sulfonamide | | |
| I-153 | 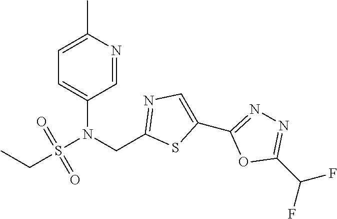 [SMILES: CCS(=O)(=O)N(Cc1ncc(-c2nnc(C(F)F)o2)s1)c1ccc(C)nc1] | 1H NMR (400 MHz, CDCl3) δ 8.22 (s, 1 H), 8.10 (s, 1 H), 7.40 (s, 2 H), 6.75 (t, J = 51.6 Hz, 1 H), 5.42 (s, 2 H), 3.09 (q, J = 7.6 Hz, 2 H), 2.18 (s, 3 H), 1.23 (t, J = 7.2 Hz, 3 H). LCMS RT = 1.666 min, m/z = 416.2 | 0.043 |
| | N-({5-[5-(difluoromethyl)-1,3,4- | | |
| | oxadiazol-2-yl]-1,3-thiazol-2- | | |
| | yl}methyl)-N-(5-methylpyridin-2- | | |
| | yl)ethane-1-sulfonamide | | |
| I-155 | 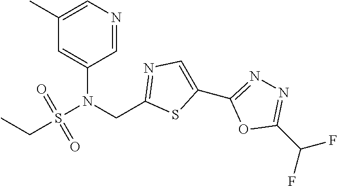 [SMILES: CCS(=O)(=O)N(Cc1ncc(-c2nnc(C(F)F)o2)s1)c1cncc(C)c1] | 1H NMR (400 MHz, CDCl3) δ 8.39 (s, 1 H), 8.29 (d, J = 4.8 Hz, 1 H), 7.47 (s, 1 H), 7.01 (d, J = 4.8 Hz, 1 H), 6.90 (t, J = 51.6 Hz, 1 H), 5.60 (s, 2 H), 3.28 (q, J = 7.6 Hz, 2 H), 2.40 (s, 3 H), 1.39 (t, J = 7.2 Hz, 3 H). LCMS RT = 1.635 min, m/z = 416.2 | 0.0528 |
| | N-({5-[5-(difluoromethyl)-1,3,4- | | |
| | oxadiazol-2-yl]-1,3-thiazol-2- | | |
| | yl}methyl)-N-(4-methylpyridin-2- | | |
| | yl)ethane-1-sulfonamide | | |
| I-156 | 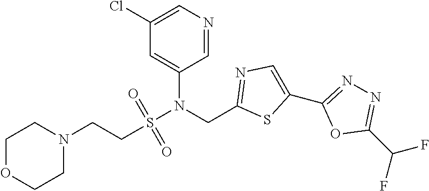 [SMILES: O=S(=O)(CCN1CCOCC1)N(Cc1ncc(-c2nnc(C(F)F)o2)s1)c1cncc(Cl)c1] | 1H NMR (400 MHz, CD3OD) δ ppm 8.68 (d, J = 2.4 Hz, 1 H), 8.52 (d, J = 2.0 Hz, 1 H), 8.44 (s, 1 H), 8.17 (t, J = 2.0 Hz, 1 H), 7.22 (t, J = 52.0 Hz, 1 H), 5.45 (s, 2 H), 3.73-3.70 (m, 4 H), 3.58 (t, J = 7.6 Hz, 2 H), 2.89 (t, J = 6.8 Hz, 2 H), 2.58-2.51 (m, 4 H). LCMS RT = 0.992 min, m/z = 521.2 | 0.00253 |
| | N-(5-chloropyridin-3-yl)-N-({5- | | |
| | [5-(difluoromethyl)-1,3,4- | | |
| | oxadiazol-2-yl]-1,3-thiazol-2- | | |
| | yl}methyl)-2-(morpholin-4- | | |
| | yl)ethane-1-sulfonamide | | |
| I-157 | 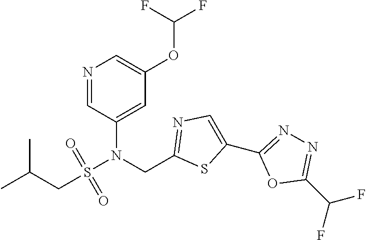 [SMILES: CC(C)CS(=O)(=O)N(Cc1ncc(-c2nnc(C(F)F)o2)s1)c1cncc(OC(F)F)c1] | 1H NMR (400 MHz, CDCl3) δ 8.59 (d, J = 2.0 Hz, 1 H), 8.47 (d, J = 2.0 Hz, 1 H), 8.40 (s, 1 H), 7.67 (t, J = 2.4 Hz, 1 H), 6.91 (t, J = 51.6 Hz, 1 H), 6.59 (t, J = 72.0 Hz, 1 H), 5.28 (s, 2 H), 3.05 (d, J = 8.0 Hz, 2 H), 2.40-2.30 (m, 1 H), 1.12 (d, J = 6.8 Hz, 6 H). LCMS RT = 0.644 min, m/z = 496.1 | 0.00693 |
| | N-[5-(difluoromethoxy)pyridin-3- | | |
| | yl]-N-({5-[5-(difluoromethyl)- | | |
| | 1,3,4-oxadiazol-2-yl]-1,3-thiazol- | | |
| | 2-yl}methyl)-2-methylpropane-1- | | |
| | sulfonamide | | |
| I-158 | 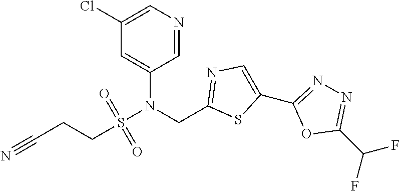 [SMILES: N#CCCS(=O)(=O)N(Cc1ncc(-c2nnc(C(F)F)o2)s1)c1cncc(Cl)c1] | 1H NMR (400 MHz, CDCl3) δ ppm 8.63 (d, J = 2.4 Hz, 1 H), 8.60 (d, J = 2.0 Hz, 1 H), 8.46 (s, 1 H), 7.91 (t, J = 2.4 Hz, 1 H), 6.93 (t, J = 51.6 Hz, 1 H), 5.30 (s, 2 H), 3.54 (t, J = 7.2 Hz, 2 H), 2.98 (t, J = 7.2 Hz, 2 H). LCMS RT = 0.576 min, m/z = 461.1 | 0.00722 |
| | N-(5-chloropyridin-3-yl)-2-cyano- | | |
| | N-({5-[5-(difluoromethyl)-1,3,4- | | |
| | oxadiazol-2-yl]-1,3-thiazol-2- | | |
| | yl}methyl)ethane-1-sulfonamide | | |
| I-159 | 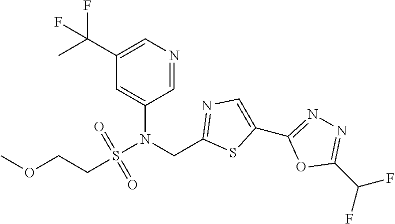 [SMILES: COCCS(=O)(=O)N(Cc1ncc(-c2nnc(C(F)F)o2)s1)c1cncc(C(C)(F)F)c1] | 1H NMR (400 MHz, CDCl3) δ 8.85 (s, 1 H), 8.56 (s, 1 H), 8.72 (s, 1 H), 8.35 (s, 1 H), 8.01 (s, 1 H), 6.91 (t, J = 51.6 Hz, 1 H), 5.26 (s, 2 H), 3.87 (t, J = 5.2 Hz, 2 H), 3.50 (s, 3 H), 3.36 (t, J = 5.2 Hz, 2 H), 1.97 (t, J = 18.4 Hz, 3 H). LCMS RT = 1.701 min, m/z = 495.9 | 0.00994 |
| | N-[5-(1,1-difluoroethyl)pyridin-3- | | |
| | yl]-N-({5-[5-(difluoromethyl)- | | |
| | 1,3,4-oxadiazol-2-yl]-1,3-thiazol- | | |
| | 2-yl}methyl)-2-methoxyethane-1- | | |
| | sulfonamide | | |
| I-160 | 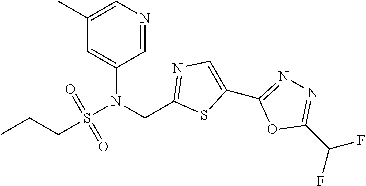 [SMILES: CCCS(=O)(=O)N(Cc1ncc(-c2nnc(C(F)F)o2)s1)c1cncc(C)c1] | 1H NMR (400 MHz, CDCl3) δ 8.49 (d, J = 2.4 Hz, 1 H), 8.41 (s, 1 H), 8.38 (s, 1 H), 7.65 (s, 1 H), 6.91 (t, J = 51.6 Hz, 1 H), 5.27 (s, 2 H), 3.16-3.08 (m, 2 H), 2.37 (s, 3 H), 1.99-1.85 (m, 2 H), 1.09 (t, J = 7.2 Hz, 3 H). LCMS RT = 1.815 min, m/z = 430.2 | 0.0109 |
| | N-({5-[5-(difluoromethyl)-1,3,4- | | |
| | oxadiazol-2-yl]-1,3-thiazol-2- | | |
| | yl}methyl)-N-(5-methylpyridin-3- | | |
| | yl)propane-1-sulfonamide | | |
| I-161 | 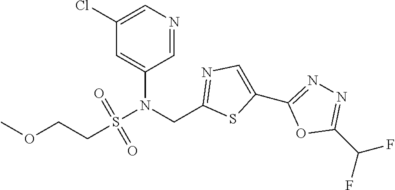 [SMILES: COCCS(=O)(=O)N(Cc1ncc(-c2nnc(C(F)F)o2)s1)c1cncc(Cl)c1] | 1H NMR (400 MHz, CDCl3) δ 8.67 (s, 1 H), 8.53 (s, 1 H), 8.35 (s, 1 H), 7.90 (t, J = 2.0 Hz, 1 H), 6.90 (t, J = 51.6 Hz, 1 H), 5.22 (s, 2 H), 3.86 (t, J = 5.6 Hz, 2 H), 3.49 (s, 3 H), 3.36 (t, J = 5.6 Hz, 2 H). LCMS RT = 1.529 min, m/z = 466.1 | 0.012 |
| | N-(5-chloropyridin-3-yl)-N-({5- | | |
| | [5-(difluoromethyl)-1,3,4- | | |
| | oxadiazol-2-yl]-1,3-thiazol-2- | | |
| | yl}methyl)-2-methoxyethane-1- | | |
| | sulfonamide | | |
| I-162 | 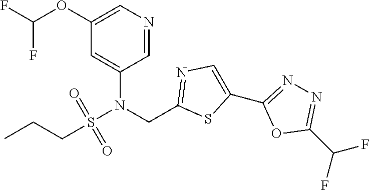 [SMILES: CCCS(=O)(=O)N(Cc1ncc(-c2nnc(C(F)F)o2)s1)c1cncc(OC(F)F)c1] | 1H NMR (400 MHz, CDCl3) δ 8.60 (s, 1 H), 8.47 (s, 1 H), 8.40 (s, 1 H), 7.67 (s, 1 H), 6.91 (t, J = 51.6 Hz, 1 H), 6.59 (t, J = 72.0 Hz, 1 H), 5.29 (s, 2 H), 3.19-3.11 (m, 2 H), 1.99- 1.86 (m, 2 H), 1.09 (t, J = 7.2 Hz, 3 H). LCMS RT = 1.794 min, m/z = 481.9 | 0.0144 |
| | N-[5-(difluoromethoxy)pyridin-3- | | |
| | yl]-N-({5-[5-(difluoromethyl)- | | |
| | 1,3,4-oxadiazol-2-yl]-1,3-thiazol- | | |
| | 2-yl}methyl)propane-1- | | |
| | sulfonamide | | |
| I-163 | 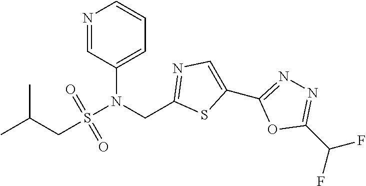 [SMILES: CC(C)CS(=O)(=O)N(Cc1ncc(-c2nnc(C(F)F)o2)s1)c1cccnc1] | 1H NMR (400 MHz, CDCl3) δ 8.70 (d, J = 2.4 Hz, 1 H), 8.60- 8.55 (m, 1 H), 8.41 (s, 1 H), 8.07-8.01 (m, 1 H), 7.53- 7.47 (m, 1 H) , 7.20 (t, J = 51.6 Hz, 1 H), 5.37 (s, 2 H), 3.20 (d, J = 6.4 Hz, 2 H), 2.34-2.22 (m, 1 H), 1.10 (d, J = 6.4 Hz, 6 H). LCMS RT = 1.423 min, m/z = 430.2 | 0.0161 |
| | N-({5-[5-(difluoromethyl)-1,3,4- | | |
| | oxadiazol-2-yl]-1,3-thiazol-2- | | |
| | yl}methyl)-2-methyl-N-(pyridin-3- | | |
| | yl)propane-1-sulfonamide | | |
| I-164 | 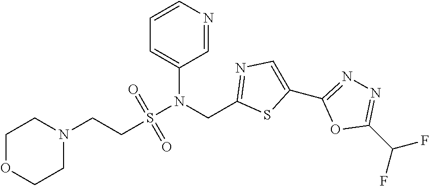 [SMILES: O=S(=O)(CCN1CCOCC1)N(Cc1ncc(-c2nnc(C(F)F)o2)s1)c1cccnc1] | 1H NMR (400 MHz, CDCl3) δ ppm 8.73 (d, J = 2.4 Hz, 1 H), 8.60-8.55 (m, 1 H), 8.38 (s, 1 H), 7.87-7.81 (m, 1 H), 7.38- 7.33 (m, 1 H), 6.91 (t, J = 51.6 Hz, 1 H), 5.32 (s, 2 H), 3.76-3.72 (m, 4 H), 3.37 (t, J = 7.2 Hz, 2 H), 2.90 (t, J = 6.8 Hz, 2 H), 2.52-2.50 (m, 4 H). LCMS RT = 0.430 min, m/z = 487.2 | 0.0173 |
| | N-({5-[5-(difluoromethyl)-1,3,4- | | |
| | oxadiazol-2-yl]-1,3-thiazol-2- | | |
| | yl}methyl)-2-(morpholin-4-yl)-N- | | |
| | (pyridin-3-yl)ethane-1- | | |
| | sulfonamide | | |
| I-165 | 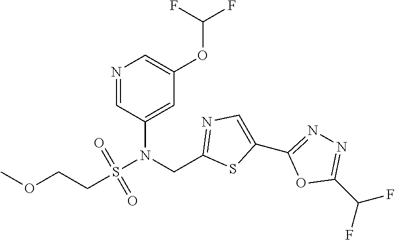 [SMILES: COCCS(=O)(=O)N(Cc1ncc(-c2nnc(C(F)F)o2)s1)c1cncc(OC(F)F)c1] | 1H NMR (400 MHz, CD3OD) δ 8.59 (s, 1 H), 8.40 (s, 2 H), 7.87 (d, J = 2.4 Hz, 1 H), 7.19 (t, J = 51.6 Hz, 1 H), 6.97 (t, J = 72.8 Hz, 1 H), 5.34 (s, 2 H), 3.83 (t, J = 5.2 Hz, 2 H), 3.52 (t, J = 5.6 Hz, 2 H), 3.30 (t, J = 1.6 Hz, 3 H). LCMS RT = 1.533 min, m/z = 498.2 | 0.0196 |
| | N-[5-(difluoromethoxy)pyridin-3- | | |
| | yl]-N-({5-[5-(difluoromethyl)- | | |
| | 1,3,4-oxadiazol-2-yl]-1,3-thiazol- | | |
| | 2-yl}methyl)-2-methoxyethane-1- | | |
| | sulfonamide | | |
| I-166 | 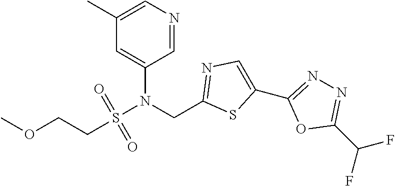 [SMILES: COCCS(=O)(=O)N(Cc1ncc(-c2nnc(C(F)F)o2)s1)c1cncc(C)c1] | 1H NMR (400 MHz, CD3OD) δ 8.49 (s, 1 H), 8.39 (s, 1 H), 8.34 (s, 1 H), 7.87 (s, 1 H), 7.20 (t, J = 51.6 Hz, 1 H), 5.31 (s, 2 H), 3.83 (t, J = 5.2 Hz, 2 H), 3.49 (t, J = 5.6 Hz, 2 H), 3.43 (s, 3 H), 2.38 (s, 3 H). LCMS RT = 2.329 min, m/z = 446.5 | 0.0266 |
| | N-({5-[5-(difluoromethyl)-1,3,4- | | |
| | oxadiazol-2-yl]-1,3-thiazol-2- | | |
| | yl}methyl)-2-methoxy-N-(5- | | |
| | methylpyridin-3-yl)ethane-1- | | |
| | sulfonamide | | |
| I-167 | 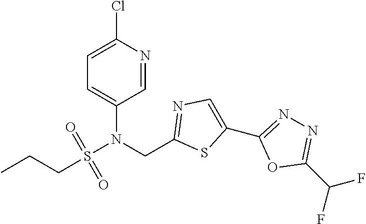 [SMILES: CCCS(=O)(=O)N(Cc1ncc(-c2nnc(C(F)F)o2)s1)c1ccc(Cl)nc1] | 1H NMR (400 MHz, CDCl3) δ 8.40-8.35 (m, 2 H), 7.73- 7.68 (m, 1 H), 7.66-7.60 (m, 1 H), 6.89 (t, J = 51.6 Hz, 1 H), 5.55 (s, 2 H), 3.26-3.17 (m, 2 H), 1.93-1.81 (m, 2 H), 1.05 (t, J = 7.4 Hz, 3 H). LCMS RT = 2.072 min, m/z = 449.8 | 0.0266 |
| | N-(5-chloropyridin-2-yl)-N-({5- | | |
| | [5-(difluoromethyl)-1,3,4- | | |
| | oxadiazol-2-yl]-1,3-thiazol-2- | | |
| | yl}methyl)propane-1-sulfonamide | | |
| I-168 | 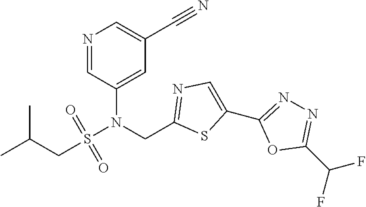 [SMILES: CC(C)CS(=O)(=O)N(Cc1ncc(-c2nnc(C(F)F)o2)s1)c1cncc(C#N)c1] | 1H NMR (400 MHz, CDCl3) δ 8.95 (d, J = 2.4 Hz, 1 H), 8.82 (s, 1 H), 8.42 (s, 1 H), 8.16 (s, 1 H), 6.92 (t, J = 52.0 Hz, 1 H), 5.28 (s, 2 H), 3.03 (d, J = 6.4 Hz, 2 H), 2.42-2.28 (m, 1 H), 1.12 (d, J = 6.8 Hz, 6 H). LCMS RT = 1.690 min, m/z = 455.2 | 0.0305 |
| | N-(5-cyanopyridin-3-yl)-N-({5-[5- | | |
| | (difluoromethyl)-1,3,4-oxadiazol- | | |
| | 2-yl]-1,3-thiazol-2-yl}methyl)-2- | | |
| | methylpropane-1-sulfonamide | | |
| I-169 | 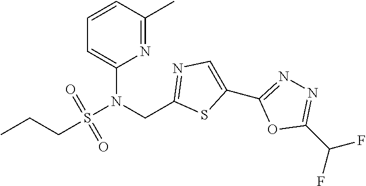 [SMILES: CCCS(=O)(=O)N(Cc1ncc(-c2nnc(C(F)F)o2)s1)c1cccc(C)n1] | 1H NMR (400 MHz, CDCl3) δ 8.38 (s, 1 H), 7.61 (t, J = 7.6 Hz, 1 H), 7.45 (d, J = 8.4 Hz, 1 H), 7.02-6.75 (m, 2 H), 5.59 (s, 2 H), 3.22-3.15 (m, 2 H), 2.52 (s, 3 H), 1.93-1.80 (m, 2 H), 1.03 (t, J = 7.6 Hz, 3 H). LCMS RT = 1.942 min, m/z = 429.9 | 0.0309 |
| | N-({5-[5-(difluoromethyl)-1,3,4- | | |
| | oxadiazol-2-yl]-1,3-thiazol-2- | | |
| | yl}methyl)-N-(6-methylpyridin-2- | | |
| | yl)propane-1-sulfonamide | | |
| I-170 | 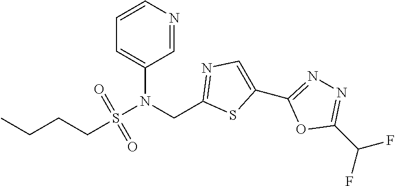 [SMILES: CCCCS(=O)(=O)N(Cc1ncc(-c2nnc(C(F)F)o2)s1)c1cccnc1] | 1H NMR (400 MHz, CDCl3) δ 8.70 (s, 1 H), 8.58 (d, J = 4.8 Hz, 1 H), 8.38 (s, 1 H), 7.90- 7.80 (m, 1 H), 7.45-7.31 (m, 1 H), 6.91 (t, J = 51.2 Hz, 1 H), 5.29 (s, 2 H), 3.21-3.06 (m, 2 H), 1.92-1.83 (m, 2 H), 1.52- 1.43 (m, 2 H), 0.96 (t, J = 7.2 Hz, 3 H). LCMS RT = 0.623 min, m/z = 429.9 | 0.0264 |
| | N-({5-[5-(difluoromethyl)-1,3,4- | | |
| | oxadiazol-2-yl]-1,3-thiazol-2- | | |
| | yl}methyl)-N-(pyridin-3- | | |
| | yl)butane-1-sulfonamide | | |
| I-171 | 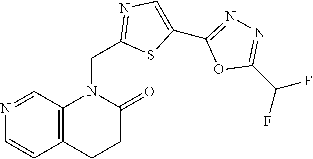 [SMILES: O=C1CCc2ccncc2N1Cc1ncc(-c2nnc(C(F)F)o2)s1] | 1H NMR (400 MHz, CDCl3) δ 8.55 (s, 1 H), 8.42 (s, 1 H), 8.31 (d, J = 3.6 Hz, 1 H), 7.16 (d, J = 4.4 Hz, 1 H), 6.89 (t, J = 51.6 Hz, 1 H), 5.52 (s, 2 H), 3.04 (t, J = 6.8 Hz, 2 H), 2.81 (t, J = 8.0 Hz, 2 H). LCMS RT = 1.398 min, m/z = 363.9 | 0.0215 |
| | 1-({5-[5-(difluoromethyl)-1,3,4- | | |
| | oxadiazol-2-yl]-1,3-thiazol-2- | | |
| | yl}methyl)-1,2,3,4-tetrahydro-1,7- | | |
| | naphthyridin-2-one | | |
| I-172 | 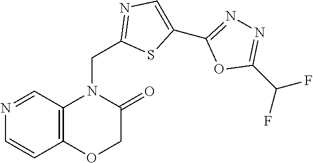 [SMILES: O=C1COc2ccncc2N1Cc1ncc(-c2nnc(C(F)F)o2)s1] | 1H NMR (400 MHz, CDCl3) δ 8.52 (s, 1 H), 8.43 (s, 1 H), 8.27 (d, J = 5.6 Hz, 1 H), 6.96 (d, J = 5.2 Hz, 1 H), 6.90 (t, J = 51.6 Hz, 1 H), 5.52 (s, 2 H), 4.85 (s, 2 H). LCMS RT = 0.418 min, m/z = 366.0 | 0.025 |
| | 4-({5-[5-(difluoromethyl)-1,3,4- | | |
| | oxadiazol-2-yl]-1,3-thiazol-2- | | |
| | yl}methyl)-2H,3H,4H-pyrido[4,3- | | |
| | b][1,4]oxazin-3-one | | |
| I-173 | 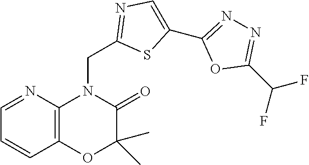 [SMILES: CC1(C)Oc2cccnc2N(Cc2ncc(-c3nnc(C(F)F)o3)s2)C1=O] | ¹H NMR (400 MHz, CDCl₃) δ 8.39 (s, 1 H), 8.01-8.05 (m, 1 H), 7.29 (dd, J = 8.1, 1.5 Hz, 1 H), 7.01 (dd, J = 8.1, 1.5 Hz, 1H), 6.89 (t, J = 51.6 Hz, 1 H), 5.70 (s, 2 H) LCMS: RT = 5.00 min, m/z = 394.0 | 0.108 |
| | 4-((5-(5-(difluoromethyl)-1,3,4- | | |
| | oxadiazol-2-yl)thiazol-2- | | |
| | yl)methyl)-2,2-dimethyl-2H- | | |
| | pyrido[3,2-b][1,4]oxazin-3(4H)- | | |
| | one | | |
| I-174 | 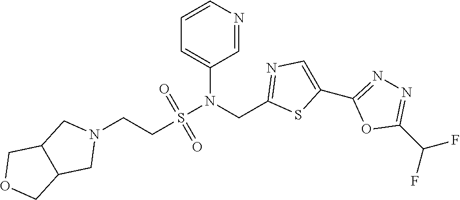 [SMILES: O=S(=O)(CCN1CC2COCC2C1)N(Cc1ncc(-c2nnc(C(F)F)o2)s1)c1cccnc1] | 1H NMR (400 MHz, CDCl3) δ 8.76 (s, 1 H), 8.58 (d, J = 4.4 Hz, 1 H), 8.39 (s, 1 H), 7.89 (d, J = 8.4 Hz, 1 H), 7.40-7.35 (m, 1 H), 6.91 (t, J = 51.6 Hz, 1 H), 5.33 (s, 2 H), 3.75-3.70 (m, 4 H), 3.49 (t, J = 6.8 Hz, 2 H), 3.07 (t, J = 6.8 Hz, 2 H), 2.99-2.96 (m, 4 H), 2.49 (d, J = 6.0 Hz, 2 H). LCMS RT = 1.435 min, m/z = 513.0 | 0.022 |
| | N-((5-(5-(difluoromethyl)-1,3,4- | | |
| | oxadiazol-2-yl)thiazol-2- | | |
| | yl)methyl)-N-(pyridin-3-yl)-2- | | |
| | (tetrahydro-1H-furo[3,4-c]pyrrol- | | |
| | 5(3H)-yl)ethane-1-sulfonamide | | |
| I-175 | 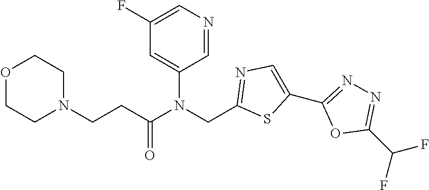 [SMILES: O=C(CCN1CCOCC1)N(Cc1ncc(-c2nnc(C(F)F)o2)s1)c1cncc(F)c1] | 1H NMR (400 MHz, CDCl3) δ 8.55 (d, J = 2.0 Hz, 1 H), 8.43- 8.36 (m, 2 H), 7.50-7.44 (m, 1 H), 6.92 (t, J = 51.6 Hz, 1 H), 5.20 (s, 2 H), 3.71-3.66 (m, 4 H), 2.81 (t, J = 6.8 Hz, 2 H), 2.50-2.22 (m, 6 H). LCMS RT = 0.400 min, m/z = 469.2 | 0.496 |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)-3-(morpholin-4- | | |
| | yl)propanamide | | |
| I-176 | 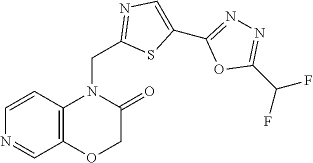 [SMILES: O=C1COc2cnccc2N1Cc1ncc(-c2nnc(C(F)F)o2)s1] | 1H NMR (400 MHz, CD3OD) δ 8.54-8.53 (m, 1 H), 8.13- 8.10 (m, 1 H), 7.22 (t, J = 51.6 Hz, 1 H), 6.97-6.89 (m, 1 H), 5.84-5.78 (m, 1 H), 4.72- 4.45 (m, 4 H). LCMS RT = 1.091 min, m/z = 365.8 | 2.52 |
| | 1-({5-[5-(difluoromethyl)-1,3,4- | | |
| | oxadiazol-2-yl]-1,3-thiazol-2- | | |
| | yl}methyl)-1H,2H,3H-pyrido[3,4- | | |
| | b][1,4]oxazin-2-one | | |
| I-177 | 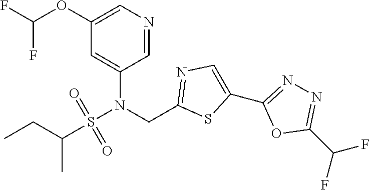 [SMILES: CCC(C)S(=O)(=O)N(Cc1ncc(-c2nnc(C(F)F)o2)s1)c1cncc(OC(F)F)c1] | 1H NMR (400 MHz, CDCl3) δ 8.59 (d, J = 2.0 Hz, 1 H), 8.45 (d, J = 2.4 Hz, 1 H), 8.37 (s, 1 H), 7.67 (t, J = 2.4 Hz, 1 H), 6.91 (t, J = 51.6 Hz, 1 H), 6.58 (t, J = 72.0 Hz, 1 H), 5.38- 5.20 (m, 2 H), 3.15-3.07 (m, 1 H), 2.13-2.02 (m, 1 H), 1.73-1.60 (m, 1 H), 1.43 (d, J = 6.8 Hz, 3 H), 1.05 (t, J = 7.2 Hz, 3 H). LCMS RT = 1.580 min, m/z = | 0.006 |
| | | 496.2 | |
| | N-[5-(difluoromethoxy)pyridin-3- | | |
| | yl]-N-({5-[5-(difluoromethyl)- | | |
| | 1,3,4-oxadiazol-2-yl]-1,3-thiazol- | | |
| | 2-yl}methyl)butane-2-sulfonamide | | |
| I-178 | 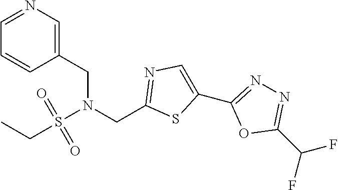 [SMILES: CCS(=O)(=O)N(Cc1cccnc1)Cc1ncc(-c2nnc(C(F)F)o2)s1] | 1H NMR (400 MHz, CDCl3) δ 8.62-8.55 (m, 2 H), 8.42 (s, 1 H), 7.88-7.82 (m, 1 H), 7.38- 7.34 (m, 1 H), 6.88 (t, J = 51.6 Hz, 1 H), 4.71 (s, 2 H), 4.60 (s, 2 H), 3.19 (q, J = 7.6 Hz, 2 H), 1.44 (t, J = 7.2 Hz, 3 H) LCMS RT = 1.031 min, m/z = 416.2 | 4.35 |
| | N-({5-[5-(difluoromethyl)-1,3,4- | | |
| | oxadiazol-2-yl]-1,3-thiazol-2- | | |
| | yl}methyl)-N-[(pyridin-3- | | |
| | yl)methyl]ethane-1-sulfonamide | | |
| I-179 | 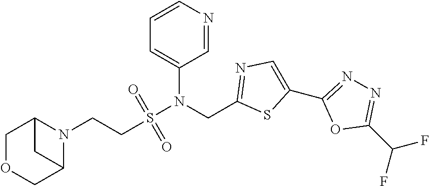 [SMILES: O=S(=O)(CCN1C2COCC1C2)N(Cc1ncc(-c2nnc(C(F)F)o2)s1)c1cccnc1] | 1H NMR (400 MHz, CDCl3) δ 8.76 (d, J = 2.4 Hz, 1 H), 8.58 (d, J = 3.2 Hz, 1 H), 8.38 (s, 1 H), 7.89-7.86 (m, 1 H), 7.39- 7.35 (m, 1 H), 6.91 (t, J = 51.6 Hz, 1 H), 5.34 (s, 2 H), 4.22 (d, J = 11.2 Hz, 2 H), 3.81 (d, J = 11.6 Hz, 2 H), 3.64 (d, J = 6.4 Hz, 2 H), 3.36-3.20 (m, 4 H), 2.74-2.69 (m, 1 H), 1.91 (d, J = 8.8 Hz, 1 H). LCMS RT = 1.438 min, m/z = | 0.013 |
| | | 499.0 | |
| | N-({5-[5-(difluoromethyl)-1,3,4- | | |
| | oxadiazol-2-yl]-1,3-thiazol-2- | | |
| | yl}methyl)-2-{3-oxa-6- | | |
| | azabicyclo[3.1.1]heptan-6-yl}-N- | | |
| | (pyridin-3-yl)ethane-1- | | |
| | sulfonamide | | |
| I-180 | 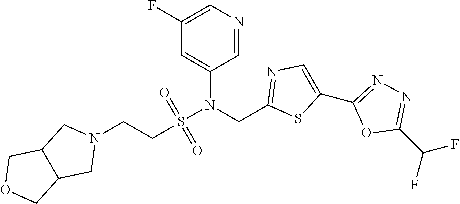 [SMILES: O=S(=O)(CCN1CC2COCC2C1)N(Cc1ncc(-c2nnc(C(F)F)o2)s1)c1cncc(F)c1] | 1H NMR (400 MHz, CDCl3) δ 8.62 (s, 1 H), 8.45 (d, J = 2.4 Hz, 1 H), 8.38 (s, 1 H), 7.75- 7.70 (m, 1 H), 6.91 (t, J = 51.6 Hz, 1 H), 5.35 (s, 2 H), 3.78- 3.69 (m, 4 H), 3.34 (t, J = 6.8 Hz, 2 H), 2.94 (t, J = 6.4 Hz, 2 H), 2.87-2.86 (m, 2 H), 2.72- 2.68 (m, 2 H), 2.49-2.46 (m, 2 H). LCMS RT = 0.417 min, m/z = 531.2 | 0.010 |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)-2-{hexahydro-1H-furo[3,4- | | |
| | c]pyrrol-5-yl}ethane-1- | | |
| | sulfonamide | | |
| I-181 | 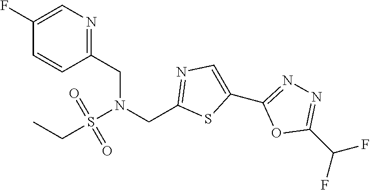 [SMILES: CCS(=O)(=O)N(Cc1ccc(F)cn1)Cc1ncc(-c2nnc(C(F)F)o2)s1] | 1H NMR (400 MHz, CDCl3) δ 8.44 (t, J = 2.0 Hz, 1 H), 8.41 (s, 1 H), 7.44 (d, J = 2.4 Hz, 1 H), 7.42 (s, 1 H), 6.92 (t, J = 51.6 Hz, 1 H), 4.86 (s, 2 H), 4.65 (s, 2 H), 3.23 (q, J = 7.2 Hz, 2 H), 1.42 (t, J = 7.2 Hz, 3 H). LCMS RT = 1.338 min, m/z = 434.2 | 3.46 |
| | N-({5-[5-(difluoromethyl)-1,3,4- | | |
| | oxadiazol-2-yl]-1,3-thiazol-2- | | |
| | yl}methyl)-N-[(5-fluoropyridin-2- | | |
| | yl)methyl]ethane-1-sulfonamide | | |
| I-182 | 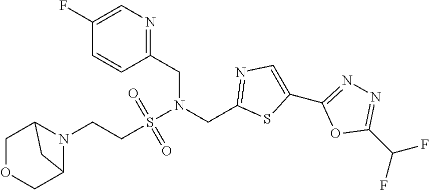 [SMILES: O=S(=O)(CCN1C2COCC1C2)N(Cc1ccc(F)cn1)Cc1ncc(-c2nnc(C(F)F)o2)s1] | 1H NMR (400 MHz, CDCl3) δ 8.61 (s, 1 H), 8.46 (d, J = 2.4 Hz, 1 H), 8.39 (s, 1 H), 7.72- 7.65 (m, 1 H), 6.91 (t, J = 51.6 Hz, 1 H), 5.35 (s, 2 H), 4.19 (d, J = 11.2 Hz, 2 H), 3.78 (d, J = 11.2 Hz, 2 H), 3.56 (d, J = 6.4 Hz, 2 H), 3.29-3.25 (m, 2 H), 3.21-3.18 (m, 2 H), 2.70- 2.65 (m, 1 H), 1.89 (d, J = 8.4 Hz, 1 H). LCMS RT = 0.844 min, m/z = | 0.018 |
| | | 517.3 | |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)-2-{3-oxa-6- | | |
| | azabicyclo[3.1.1]heptan-6- | | |
| | yl}ethane-1-sulfonamide | | |
| I-183 | 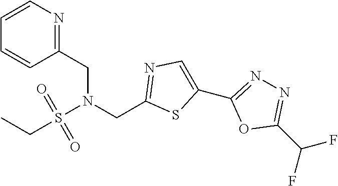 [SMILES: CCS(=O)(=O)N(Cc1ccccn1)Cc1ncc(-c2nnc(C(F)F)o2)s1] | 1H NMR (400 MHz, CDCl3) δ 8.59 (d, J = 4.4 Hz, 1 H), 8.40 (s, 1 H), 7.76-7.69 (m, 1 H), 7.39 (d, J = 7.6 Hz, 1 H), 7.25 (s, 1 H), 6.92 (t, J = 51.6 Hz, 1 H), 4.89 (s, 2 H), 4.68 (s, 2 H), 3.25 (q, J = 7.2 Hz, 2 H), 1.42 (t, J = 7.2 Hz, 3 H). LCMS RT = 0.932 min, m/z = 416.2 | 2.54 |
| | N-({5-[5-(difluoromethyl)-1,3,4- | | |
| | oxadiazol-2-yl]-1,3-thiazol-2- | | |
| | yl}methyl)-N-[(pyridin-2- | | |
| | yl)methyl]ethane-1-sulfonamide | | |
| I-184 | 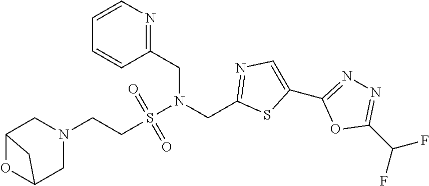 [SMILES: O=S(=O)(CCN1CC2CC(C1)O2)N(Cc1ccccn1)Cc1ncc(-c2nnc(C(F)F)o2)s1] | 1H NMR (400 MHz, CDCl3) δ 8.73 (d, J = 2.4 Hz, 1 H), 8.60 (d, J = 3.2 Hz, 1 H), 8.39 (s, 1 H), 7.89-7.82 (m, 1 H), 7.40- 7.33 (m, 1 H), 6.91 (t, J = 51.6 Hz, 1 H), 5.31 (s, 2 H), 4.53 (d, J = 6.4 Hz, 2 H), 3.46- 3.42 (m, 2 H), 3.21-3.02 (m, 5 H), 2.86 (d, J = 11.2 Hz, 2 H), 2.29 (d, J = 8.4 Hz, 1 H). LCMS RT = 1.352 min, m/z = 498.9 | 0.014 |
| | N-({5-[5-(difluoromethyl)-1,3,4- | | |
| | oxadiazol-2-yl]-1,3-thiazol-2- | | |
| | yl}methyl)-2-{6-oxa-3- | | |
| | azabicyclo[3.1.1]heptan-3-yl}-N- | | |
| | (pyridin-3-yl)ethane-1- | | |
| | sulfonamide | | |
| I-185 | 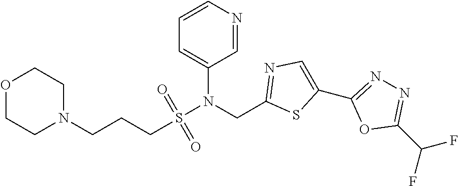 [SMILES: O=S(=O)(CCCN1CCOCC1)N(Cc1ncc(-c2nnc(C(F)F)o2)s1)c1cccnc1] | 1H NMR (400 MHz, CDCl3) δ 8.65 (d, J = 4.4 Hz, 1 H), 8.55 (d, J = 2.4 Hz, 1 H), 8.38 (s, 1 H), 7.63 (d, J = 8.0 Hz, 1 H), 7.43-7.39 (m, 1 H), 6.92 (t, J = 51.6 Hz, 1 H), 5.21 (s, 2 H), 3.65 (t, J = 4.4 Hz, 4 H), 2.73 (t, J = 7.2 Hz, 2 H), 2.38-2.33 (m, 6 H). LCMS RT = 0.411 min, m/z = 451.2 | 0.516 |
| | N-({5-[5-(difluoromethyl)-1,3,4- | | |
| | oxadiazol-2-yl]-1,3-thiazol-2- | | |
| | yl}methyl)-3-(morpholin-4-yl)-N- | | |
| | (pyridin-3-yl)propanamide | | |
| I-186 | 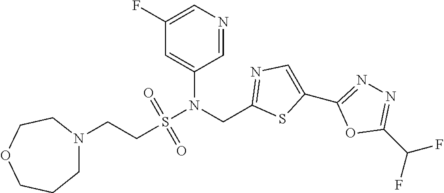 [SMILES: O=S(=O)(CCN1CCCOCC1)N(Cc1ncc(-c2nnc(C(F)F)o2)s1)c1cncc(F)c1] | 1H NMR (400 MHz, CDCl3) δ 8.58 (s, 1 H), 8.47 (d, J = 2.4 Hz, 1 H), 8.40 (s, 1 H), 7.70- 7.67 (m, 1 H), 6.91 (t, J = 51.6 Hz, 1 H), 5.32 (s, 2 H), 3.81 (t, J = 6.4 Hz, 2 H), 3.75 (t, J = 4.8 Hz, 2 H), 3.41 (t, J = 7.2 Hz, 2 H), 3.12 (t, J = 7.6 Hz, 2 H), 2.84-2.76 (m, 4 H), 1.97- 1.92 (m, 2 H). LCMS RT = 0.414 min, m/z = 519.2 | 0.0276 |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)-2-(1,4-oxazepan-4-yl)ethane-1- | | |
| | sulfonamide | | |
| I-187 | 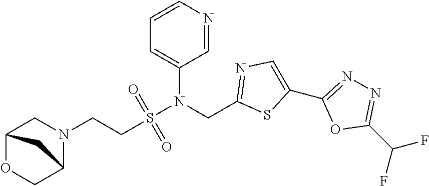 [SMILES: O=S(=O)(CCN1C[C@@H]2C[C@H]1CO2)N(Cc1ncc(-c2nnc(C(F)F)o2)s1)c1cccnc1] | 1H NMR (400 MHz, CDCl3) δ 8.74 (d, J = 2.4 Hz, 1 H), 8.60- 8.57 (m, 1 H), 8.38 (s, 1 H), 7.88-7.85 (m, 1 H), 7.40- 7.26 (m, 1 H), 6.91 (t, J = 51.6 Hz, 1 H), 5.37-5.25 (m, 2 H), 4.47 (s, 1 H), 4.02 (d, J = 8.4 Hz, 1 H), 3.70-3.55 (m, 1 H), 3.58 (s, 1 H), 3.36 (t, J = 6.8 Hz, 2 H), 3.22-3.09 (m, 2 H), 3.00-2.92 (m, 1 H), 2.60 (d, J = 9.6 Hz, 1 H), 1.86-1.80 (m, 2 H). | 0.0247 |
| | | LCMS RT = 0.429 min, m/z = | |
| | N-({5-[5-(difluoromethyl)-1,3,4- | 499.2 | |
| | oxadiazol-2-yl]-1,3-thiazol-2- | | |
| | yl}methyl)-2-[(1S,4S)-2-oxa-5- | | |
| | azabicyclo[2.2.1]heptan-5-yl]-N- | | |
| | (pyridin-3-yl)ethane-1- | | |
| | sulfonamide | | |
| I-188 | 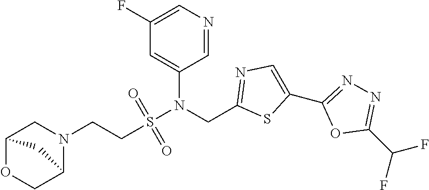 [SMILES: O=S(=O)(CCN1C[C@H]2C[C@@H]1CO2)N(Cc1ncc(-c2nnc(C(F)F)o2)s1)c1cncc(F)c1] | 1H NMR (400 MHz, CDCl3) δ 8.58 (s, 1 H), 8.47 (d, J = 2.4 Hz, 1 H), 8.39 (s, 1 H), 7.69- 7.66 (m, 1 H), 6.91 (t, J = 51.6 Hz, 1 H), 5.37-5.27 (m, 2 H), 4.47 (s, 1 H), 4.02 (d, J = 8.0 Hz, 1 H), 3.68 (dd, J = 8.4, 1.6 Hz, 1 H), 3.58 (s, 1 H), 3.38 (t, J = 6.8 Hz, 2 H), 3.22-3.09 (m, 2 H), 2.98-2.96 (m, 1 H), 2.60 (d, J = 10.0 Hz, 1 H), 1.85- 1.83 (m, 2 H). | 0.0151 |
| | | LCMS RT = 0.457 min, m/z = | |
| | N-({5-[5-(difluoromethyl)-1,3,4- | 517.2 | |
| | oxadiazol-2-yl]-1,3-thiazol-2- | | |
| | yl}methyl)-N-(5-fluoropyridin-3- | | |
| | yl)-2-[(1R,4R)-2-oxa-5- | | |
| | azabicyclo[2.2.1]heptan-5- | | |
| | yl]ethane-1-sulfonamide | | |
| I-189 | 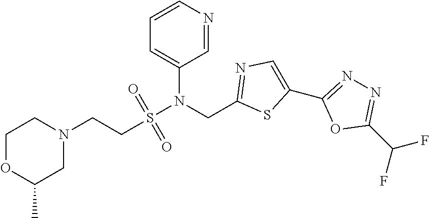 [SMILES: C[C@H]1CN(CCS(=O)(=O)N(Cc2ncc(-c3nnc(C(F)F)o3)s2)c2cccnc2)CCO1] | 1H NMR (400 MHz, CDCl3) δ 8.73 (d, J = 2.4 Hz, 1 H), 8.59 (d, J = 3.6 Hz, 1 H), 8.38 (s, 1 H), 7.88-7.85 (m, 1 H), 7.39- 7.35 (m, 1 H), 6.91 (t, J = 51.6 Hz, 1 H), 5.32 (s, 2 H), 3.91-3.88 (m, 1 H), 3.71- 3.62 (m, 2 H), 3.38 (t, J = 6.8 Hz, 2 H), 2.90 (t, J = 7.2 Hz, 2 H), 2.76-2.69 (m, 2 H), 2.28- 2.21 (m, 1 H), 1.93 (t, J = 10.8 Hz, 1 H), 1.17 (d, J = 6.0 Hz, 3 H). LCMS RT = 1.427 min, m/z = | 0.0363 |
| | | 500.9 | |
| | N-({5-[5-(difluoromethyl)-1,3,4- | | |
| | oxadiazol-2-yl]-1,3-thiazol-2- | | |
| | yl}methyl)-2-[(2S)-2- | | |
| | methylmorpholin-4-yl]-N- | | |
| | (pyridin-3-yl)ethane-1- | | |
| | sulfonamide | | |
| I-190 | 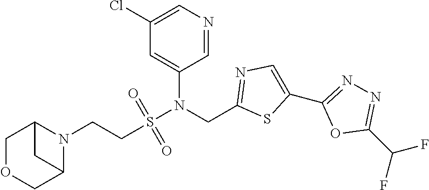 [SMILES: O=S(=O)(CCN1C2COCC1C2)N(Cc1ncc(-c2nnc(C(F)F)o2)s1)c1cncc(Cl)c1] | 1H NMR (400 MHz, CDCl3) δ 8.65 (d, J = 2.4 Hz, 1 H), 8.54 (d, J = 2.4 Hz, 1 H), 8.39 (s, 1 H), 7.95 (t, J = 2.4 Hz, 1 H), 6.91 (t, J = 51.6 Hz, 1 H), 5.34 (s, 2 H), 4.21 (d, J = 11.6 Hz, 2 H), 3.80 (d, J = 11.2 Hz, 2 H), 3.60 (d, J = 6.0 Hz, 2 H), 3.32- 3.25 (m, 2 H), 3.24-3.17 (m, 2 H), 2.77-2.63 (m, 1 H), 1.91 (d, J = 8.4 Hz, 1 H). LCMS RT = 0.968 min, m/z = 533.2 | 0.016 |
| | N-(5-chloropyridin-3-yl)-N-({5- | | |
| | [5-(difluoromethyl)-1,3,4- | | |
| | oxadiazol-2-yl]-1,3-thiazol-2- | | |
| | yl}methyl)-2-{3-oxa-6- | | |
| | azabicyclo[3.1.1]heptan-6- | | |
| | yl}ethane-1-sulfonamide | | |
| I-191 | 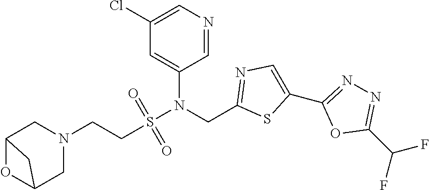 [SMILES: O=S(=O)(CCN1CC2CC(C1)O2)N(Cc1ncc(-c2nnc(C(F)F)o2)s1)c1cncc(Cl)c1] | 1H NMR (400 MHz, CDCl3) δ 8.62 (d, J = 2.0 Hz, 1 H), 8.55 (d, J = 2.0 Hz, 1 H), 8.41 (s, 1 H), 7.90 (t, J = 2.4 Hz, 1 H), 6.91 (t, J = 51.2 Hz, 1 H), 5.29 (s, 2 H), 4.52 (d, J = 6.4 Hz, 2 H), 3.43 (t, J = 6.8 Hz, 2 H), 3.18 (t, J = 7.2 Hz, 2 H), 3.10 (d, J = 10.8 Hz, 2 H), 3.06- 2.80 (m, 3 H), 2.28 (d, J = 8.0 Hz, 1 H). LCMS RT = 0.482 min, m/z = 533.2 | 0.0124 |
| | N-(5-chloropyridin-3-yl)-N-({5- | | |
| | [5-(difluoromethyl)-1,3,4- | | |
| | oxadiazol-2-yl]-1,3-thiazol-2- | | |
| | yl}methyl)-2-{6-oxa-3- | | |
| | azabicyclo[3.1.1]heptan-3- | | |
| | yl}ethane-1-sulfonamide | | |
| I-192 | 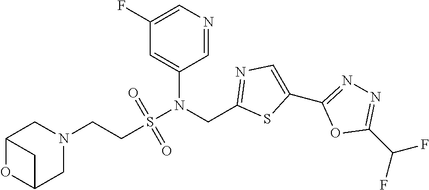 [SMILES: O=S(=O)(CCN1CC2CC(C1)O2)N(Cc1ncc(-c2nnc(C(F)F)o2)s1)c1cncc(F)c1] | 1H NMR (400 MHz, CDCl3) δ 8.57 (s, 1 H), 8.46 (d, J = 2.4 Hz, 1 H), 8.40 (s, 1 H), 7.73- 7.61 (m, 1 H), 6.92 (t, J = 51.2 Hz, 1 H), 5.31 (s, 2 H), 4.52 (d, J = 6.0 Hz, 2 H), 3.46 (t, J = 6.8 Hz, 2 H), 3.18 (t, J = 7.6 Hz, 2 H), 3.12 (d, J = 11.0 Hz, 2 H), 3.08-3.01 (m, 1 H), 2.86 (d, J = 11.2 Hz, 2 H), 2.27 (d, J = 8.0 Hz, 1 H). LCMS RT = 1.368 min, m/z = 517.2 | 0.0168 |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)-2-{6-oxa-3- | | |
| | azabicyclo[3.1.1]heptan-3- | | |
| | yl}ethane-1-sulfonamide | | |
| I-193 | 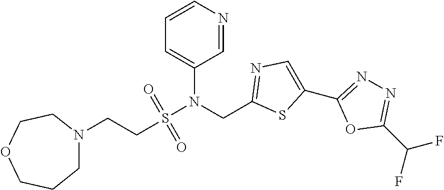 [SMILES: O=S(=O)(CCN1CCCOCC1)N(Cc1ncc(-c2nnc(C(F)F)o2)s1)c1cccnc1] | 1H NMR (400 MHz, CDCl3) δ ppm 8.73 (d, J = 2.4 Hz, 1 H), 8.58 (d, J = 4.0 Hz, 1 H), 8.38 (s, 1 H), 7.86 (d, J = 8.0 Hz, 1 H), 7.39-7.35 (m, 1 H), 6.92 (t, J = 51.6 Hz, 1 H), 5.32 (s, 2 H), 3.80 (t, J = 6.0 Hz, 2 H), 3.75-3.73 (m, 2 H), 3.37 (t, J = 7.6 Hz, 2 H), 3.10 (t, J = 6.4 Hz, 2 H), 2.79-2.75 (m, 4 H), 1.95-1.91 (m, 2 H). LCMS RT = 0.429 min, m/z = 501.2 | 0.0203 |
| | N-({5-[5-(difluoromethyl)-1,3,4- | | |
| | oxadiazol-2-yl]-1,3-thiazol-2- | | |
| | yl}methyl)-2-(1,4-oxazepan-4-yl)- | | |
| | N-(pyridin-3-yl)ethane-1- | | |
| | sulfonamide | | |
| I-194 | 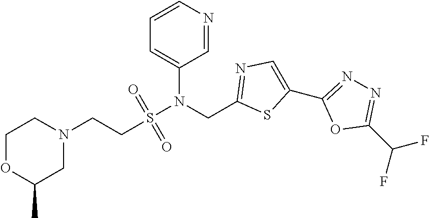 [SMILES: C[C@@H]1CN(CCS(=O)(=O)N(Cc2ncc(-c3nnc(C(F)F)o3)s2)c2cccnc2)CCO1] | 1H NMR (400 MHz, CDCl3) δ ppm 8.73 (s, 1 H), 8.59 (d, J = 4.4 Hz, 1 H), 8.38 (s, 1 H), 7.86-7.83 (m, 1 H), 7.38- 7.35 (m, 1 H), 6.91 (t, J = 51.2 Hz, 1 H), 5.31 (s, 2 H), 3.87- 3.67 (m, 1 H), 3.66-3.64 (m, 2 H), 3.64 (t, J = 6.8 Hz, 2 H), 2.89 (t, J = 6.8 Hz, 2 H), 2.74- 2.67 (m, 2 H), 2.24-2.23 (m, 1 H), 1.92 (t, J = 6.8 Hz, 1 H), 1.17 (d, J = 6.8 Hz, 3 H). LCMS RT = 0.748 min, m/z = 501.2 | 0.0267 |
| | N-({5-[5-(difluoromethyl)-1,3,4- | | |
| | oxadiazol-2-yl]-1,3-thiazol-2- | | |
| | yl}methyl)-2-[(2R)-2- | | |
| | methylmorpholin-4-yl]-N- | | |
| | (pyridin-3-yl)ethane-1- | | |
| | sulfonamide | | |
| I-195 | 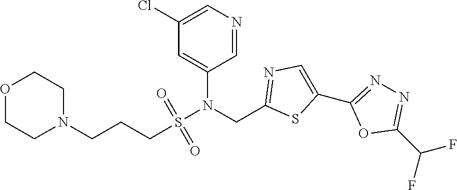 [SMILES: O=S(=O)(CCCN1CCOCC1)N(Cc1ncc(-c2nnc(C(F)F)o2)s1)c1cncc(Cl)c1] | 1H NMR (400 MHz, CDCl3) δ ppm 8.62 (s, 1 H), 8.44 (s, 1 H), 8.39 (s, 1 H), 7.72 (s, 1 H), 6.92 (t, J = 51.6 Hz, 1 H), 5.18 (s, 2 H), 3.17-3.69 (m, 8 H), 2.86-2.83 (m, 2 H), 2.44- 2.41 (m, 2 H). LCMS RT = 1.381 min, m/z = 485.3 | 0.312 |
| | N-(5-chloropyridin-3-yl)-N-({5- | | |
| | [5-(difluoromethyl)-1,3,4- | | |
| | oxadiazol-2-yl]-1,3-thiazol-2- | | |
| | yl}methyl)-3-(morpholin-4- | | |
| | yl)propanamide | | |
| I-196 | 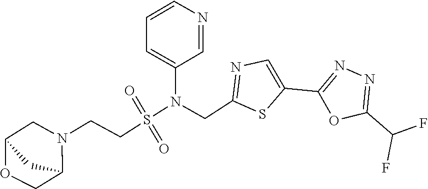 [SMILES: O=S(=O)(CCN1C[C@H]2C[C@@H]1CO2)N(Cc1ncc(-c2nnc(C(F)F)o2)s1)c1cccnc1] | 1H NMR (400 MHz, CDCl3) δ 8.74 (s, 1 H), 8.59 (d, J = 4.0 Hz, 1 H), 8.39 (s, 1 H), 7.87 (d, J = 8.4 Hz, 1 H), 7.39-7.35 (m, 1 H), 6.91 (t, J = 51.6 Hz, 1 H), 5.36-5.27 (m, 2 H), 4.48 (s, 1 H), 4.05 (d, J = 8.4 Hz, 1 H), 3.70-3.66 (m, 2 H), 3.43 (t, J = 6.8 Hz, 2 H), 3.26- 3.13 (m, 2 H), 3.03 (d, J = 10.0 Hz, 1 H), 2.66 (d, J = 10.4 Hz, 1 H), 1.95-1.85 (m, 2 H). | 0.0497 |
| | | LCMS RT = 1.117 min, m/z = | |
| | N-({5-[5-(difluoromethyl)-1,3,4- | 499.2 | |
| | oxadiazol-2-yl]-1,3-thiazol-2- | | |
| | yl}methyl)-2-[(1R,4R)-2-oxa-5- | | |
| | azabicyclo[2.2.1]heptan-5-yl]-N- | | |
| | (pyridin-3-yl)ethane-1- | | |
| | sulfonamide | | |
| I-197 | 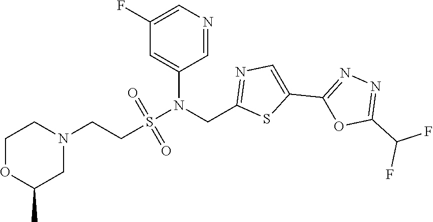 | 1H NMR (400 MHz, CDCl3) δ 8.63 (d, J = 2.0 Hz, 1 H), 8.54 (d, J = 2.0 Hz, 1 H), 8.39 (s, 1 H), 7.91 (t, J = 2.0 Hz, 1 H), 6.91 (t, J = 51.6 Hz, 1 H), 5.30 (s, 2 H), 3.93-3.85 (m, 1 H), 3.72-3.60 (m, 2 H), 3.36 (t, J = 6.8 Hz, 2 H), 2.88 (t, J = 6.8 Hz, 2 H), 2.75-2.65 (m, 2 H), 2.28-2.17 (m, 1 H), 1.91 (t, J = 10.8 Hz, 1 H), 1.17 (d, J = 6.4 Hz, 3 H). LCMS RT = 1.558 min, m/z = 535.2 | 0.0472 |
| | N-(5-chloropyridin-3-yl)-N-({5- | | |
| | [5-(difluoromethyl)-1,3,4- | | |
| | oxadiazol-2-yl]-1,3-thiazol-2- | | |
| | yl}methyl)-2-[(2R)-2- | | |
| | methylmorpholin-4-yl]ethane-1- | | |
| | sulfonamide | | |
| I-198 | 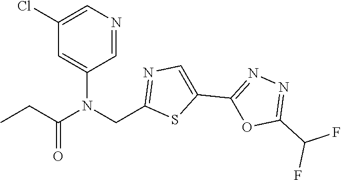 [SMILES: CCC(=O)N(Cc1ncc(-c2nnc(C(F)F)o2)s1)c1cncc(Cl)c1] | 1H NMR (400 MHz, CDCl3) δ 8.62 (s, 1 H), 8.44-8.35 (m, 2 H), 7.67 (t, J = 2.0 Hz, 1 H), 6.92 (t, J = 51.6 Hz, 1 H), 5.18 (s, 2 H), 2.18 (q, J = 7.2 Hz, 2 H), 1.13 (t, J = 7.6 Hz, 3 H). LCMS RT = 1.929 min, m/z = 400.2 | 0.299 |
| | N-(5-chloropyridin-3-yl)-N-({5- | | |
| | [5-(difluoromethyl)-1,3,4- | | |
| | oxadiazol-2-yl]-1,3-thiazol-2- | | |
| | yl}methyl)propanamide | | |
| I-199 | 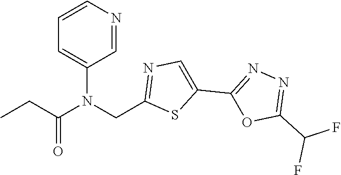 [SMILES: CCC(=O)N(Cc1ncc(-c2nnc(C(F)F)o2)s1)c1cccnc1] | 1H NMR (400 MHz, CDCl3) δ 8.64 (d, J = 4.0 Hz, 1 H), 8.53 (s, 1 H), 8.37 (s, 1 H), 7.61 (d, J = 8.0 Hz, 1 H), 7.42-7.36 (m, 1 H), 6.91 (t, J = 51.6 Hz, 1 H), 5.20 (s, 2 H), 2.15 (q, J = 7.2 Hz, 2 H), 1.12 (t, J = 7.2 Hz, 3 H). LCMS RT = 1.372 min, m/z = 366.2 | 0.355 |
| | N-({5-[5-(difluoromethyl)-1,3,4- | | |
| | oxadiazol-2-yl]-1,3-thiazol-2- | | |
| | yl}methyl)-N-(pyridin-3- | | |
| | yl)propanamide | | |
| I-200 | 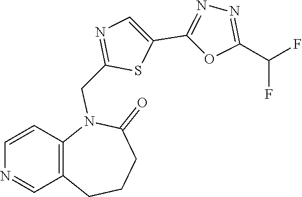 [SMILES: O=C1CCCc2cnccc2N1Cc1ncc(-c2nnc(C(F)F)o2)s1] | 1H NMR (400 MHz, CDCl3) δ 8.56 (d, J = 5.2 Hz, 1 H), 8.46 (s, 1 H), 8.39 (s, 1 H), 7.36 (d, J = 5.6 Hz, 1 H), 6.91 (t, J = 51.6 Hz, 1 H), 5.33 (s, 2 H), 2.80 (t, J = 7.2 Hz, 2 H), 2.44 (t, J = 7.2 Hz, 2 H), 2.36-2.24 (m, 2 H). LCMS RT = 0.993 min, m/z = 378.2 | 0.125 |
| | 1-({5-[5-(difluoromethyl)-1,3,4- | | |
| | oxadiazol-2-yl]-1,3-thiazol-2- | | |
| | yl}methyl)-1H,2H,3H,4H,5H- | | |
| | pyrido[4,3-b]azepin-2-one | | |
| I-201 | 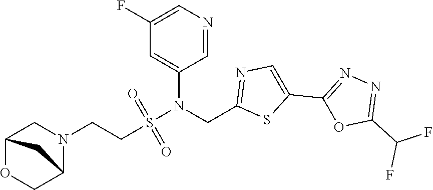 [SMILES: O=S(=O)(CCN1C[C@@H]2C[C@H]1CO2)N(Cc1ncc(-c2nnc(C(F)F)o2)s1)c1cncc(F)c1] | 1H NMR (400 MHz, CDCl3) δ 8.57 (s, 1 H), 8.46 (d, J = 2.4 Hz, 1 H), 8.39 (s, 1 H), 7.70- 7.62 (m, 1 H), 6.91 (t, J = 51.6 Hz, 1 H), 5.39-5.25 (m, 2 H), 4.46 (s, 1 H), 4.00 (d, J = 8.0 Hz, 1 H), 3.69-3.64 (m, 1 H), 3.52 (s, 1 H), 3.34 (t, J = 6.8 Hz, 2 H), 3.19-3.07 (m, 2 H), 2.94 (d, J = 10.0 Hz, 1 H), 2.56 (d, J = 10.0 Hz, 1 H), 1.89- 1.79 (m, 2 H). | 0.0207 |
| | | LCMS RT = 0.459 min, m/z = | |
| | N-({5-[5-(difluoromethyl)-1,3,4- | 517.2 | |
| | oxadiazol-2-yl]-1,3-thiazol-2- | | |
| | yl}methyl)-N-(5-fluoropyridin-3- | | |
| | yl)-2-[(1S,4S)-2-oxa-5- | | |
| | azabicyclo[2.2.1]heptan-5- | | |
| | yl]ethane-1-sulfonamide | | |
| I-202 | 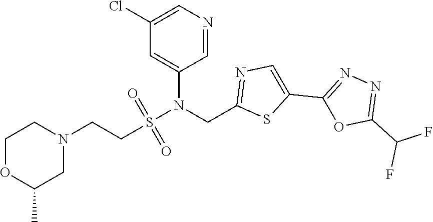 [SMILES: C[C@H]1CN(CCS(=O)(=O)N(Cc2ncc(-c3nnc(C(F)F)o3)s2)c2cncc(Cl)c2)CCO1] | 1H NMR (400 MHz, CDCl3) δ 8.63 (s, 1 H), 8.54 (s, 1 H), 8.39 (s, 1 H), 7.91 (s, 1 H), 6.91 (t, J = 51.2 Hz, 1 H), 5.30 (s, 2 H), 3.94-3.85 (m, 1 H), 3.74-3.62 (m, 2 H), 3.43 (t, J = 6.8 Hz, 2 H), 2.88 (t, J = 6.8 Hz, 2 H), 2.72 (t, J = 12.8 Hz, 2 H), 2.28-2.17 (m, 1 H), 1.92 (t, J = 9.6 Hz, 1 H), 1.17 (d, J = 6.0 Hz, 3 H). LCMS RT = 1.561 min, m/z = 535.2 | 0.0127 |
| | N-(5-chloropyridin-3-yl)-N-({5- | | |
| | [5-(difluoromethyl)-1,3,4- | | |
| | oxadiazol-2-yl]-1,3-thiazol-2- | | |
| | yl}methyl)-2-[(2S)-2- | | |
| | methylmorpholin-4-yl]ethane-1- | | |
| | sulfonamide | | |
| I-203 | 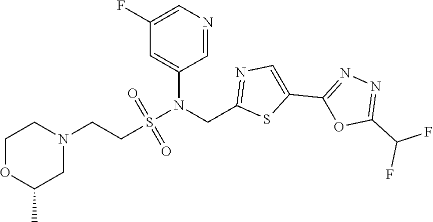 [SMILES: C[C@H]1CN(CCS(=O)(=O)N(Cc2ncc(-c3nnc(C(F)F)o3)s2)c2cncc(F)c2)CCO1] | 1H NMR (400 MHz, CDCl3) δ 8.58 (s, 1 H), 8.47 (d, J = 2.4 Hz, 1 H), 8.40 (s, 1 H), 7.68 (d, J = 9.2 Hz, 1 H), 6.92 (t, J = 51.2 Hz, 1 H), 5.32 (s, 2 H), 3.91-3.71 (m, 1 H), 3.72- 3.64 (m, 2 H), 3.41 (t, J = 6.8 Hz, 2 H), 2.91 (t, J = 7.2 Hz, 2 H), 2.73 (t, J = 12.8 Hz, 2 H), 2.29-2.22 (m, 1 H), 1.96- 1.94 (m, 1 H), 1.17 (d, J = 6.4 Hz, 3 H). LCMS RT = 1.660 min, m/z = 519.0 | 0.0254 |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)-2-[(2S)-2-methylmorpholin-4- | | |
| | yl]ethane-1-sulfonamide | | |
| I-204 | 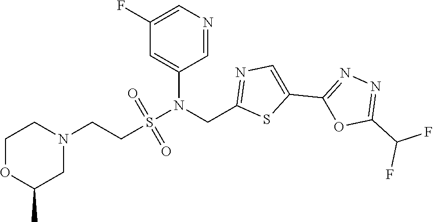 [SMILES: C[C@@H]1CN(CCS(=O)(=O)N(Cc2ncc(-c3nnc(C(F)F)o3)s2)c2cncc(F)c2)CCO1] | 1H NMR (400 MHz, CDCl3) δ 8.58 (s, 1 H), 8.47 (s, 1 H), 8.40 (s, 1 H), 7.70-7.66 (m, 1 H), 6.91 (t, J = 51.6 Hz, 1 H), 5.32 (s, 2 H), 3.89 (d, J = 9.6 Hz, 1 H), 3.70-3.60 (m, 2 H), 3.37 (t, J = 6.8 Hz, 2 H), 2.88 (t, J = 6.8 Hz, 2 H), 2.72-2.65 (m, 2 H), 2.26-2.20 (m, 1 H), 1.91 (t, J = 10.4 Hz, 1 H), 1.17 (d, J = 6.0 Hz, 3 H). LCMS RT = 1.664 min, m/z = 518.9 | 0.0336 |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)-2-[(2R)-2-methylmorpholin-4- | | |
| | yl]ethane-1-sulfonamide | | |
| I-205 | 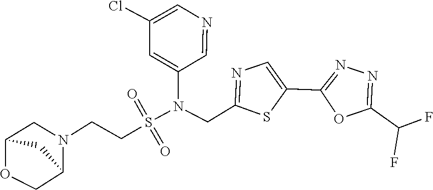 [SMILES: O=S(=O)(CCN1C[C@H]2C[C@@H]1CO2)N(Cc1ncc(-c2nnc(C(F)F)o2)s1)c1cncc(Cl)c1] | 1H NMR (400 MHz, CDCl3) δ 8.62 (d, J = 2.0 Hz, 1 H), 8.54 (d, J = 2.0 Hz, 1 H), 8.40 (s, 1 H), 7.91 (t, J = 2.0 Hz, 1 H), 6.91 (t, J = 51.6 Hz, 1 H), 5.40- 5.21 (m, 2 H), 4.47 (s, 1 H), 4.02 (d, J = 8.0 Hz, 1 H), 3.68- 3.63 (m, 1 H), 3.56 (s, 1 H), 3.35 (t, J = 6.8 Hz, 2 H), 3.25- 3.05 (m, 2 H), 3.01-2.90 (m, 1 H), 2.59 (d, J = 10.0 Hz, 1 H), 1.93-1.80 (m, 2 H). | 0.0159 |
| | | LCMS RT = 0.919 min, m/z = | |
| | N-(5-chloropyridin-3-yl)-N-({5- | 533.2 | |
| | [5-(difluoromethyl)-1,3,4- | | |
| | oxadiazol-2-yl]-1,3-thiazol-2- | | |
| | yl}methyl)-2-[(1R,4R)-2-oxa-5- | | |
| | azabicyclo[2.2.1]heptan-5- | | |
| | yl]ethane-1-sulfonamide | | |
| I-206 | 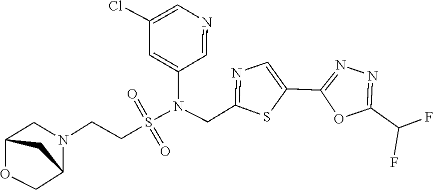 [SMILES: O=S(=O)(CCN1C[C@@H]2C[C@H]1CO2)N(Cc1ncc(-c2nnc(C(F)F)o2)s1)c1cncc(Cl)c1] | 1H NMR (400 MHz, CDCl3) δ 8.62 (d, J = 2.0 Hz, 1 H), 8.54 (d, J = 2.0 Hz, 1 H), 8.39 (s, 1 H), 7.91 (t, J = 2.0 Hz, 1 H), 6.91 (t, J = 51.6 Hz, 1 H), 5.37- 5.25 (m, 2 H), 4.46 (s, 1 H), 4.01 (d, J = 8.4 Hz, 1 H), 3.69- 3.62 (m, 1 H), 3.51 (s, 1 H), 3.31 (t, J = 6.8 Hz, 2 H), 3.18- 3.07 (m, 2 H), 2.93 (d, J = 10.0 Hz, 1 H), 2.56 (d, J = 9.6 Hz, 1 H), 1.89-1.84 (q, J = 7.2 Hz, 2 H). | 0.0162 |
| | | LCMS RT = 0.472 min, m/z = | |
| | N-(5-chloropyridin-3-yl)-N-({5- | 533.2 | |
| | [5-(difluoromethyl)-1,3,4- | | |
| | oxadiazol-2-yl]-1,3-thiazol-2- | | |
| | yl}methyl)-2-[(1S,4S)-2-oxa-5- | | |
| | azabicyclo[2.2.1]heptan-5- | | |
| | yl]ethane-1-sulfonamide | | |
| I-207 | 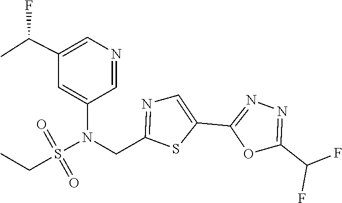 [SMILES: CCS(=O)(=O)N(Cc1ncc(-c2nnc(C(F)F)o2)s1)c1cncc([C@H](C)F)c1] | 1H NMR (400 MHz, CDCl3) δ 8.67 (d, J = 2.4 Hz, 1 H), 8.56 (s, 1 H), 8.39 (s, 1 H), 7.83 (s, 1 H), 6.91 (t, J = 51.6 Hz, 1 H), 5.79-5.60 (m, 1 H), 5.30 (s, 2 H), 3.21 (q, J = 7.2 Hz, 2 H), 1.69-1.55 (m, 3 H), 1.46 (t, J = 7.6 Hz, 3 H). LCMS RT = 1.500 min, m/z = 447.8 | 0.0137 |
| | N-({5-[5-(difluoromethyl)-1,3,4- | | |
| | oxadiazol-2-yl]-1,3-thiazol-2- | | |
| | yl}methyl)-N-{5-[(1S)-1- | | |
| | fluoroethyl]pyridin-3-yl}ethane-1- | | |
| | sulfonamide | | |
| I-208 | 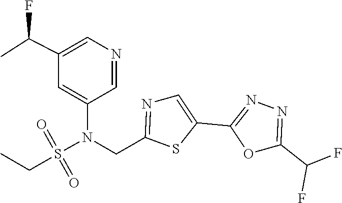 [SMILES: CCS(=O)(=O)N(Cc1ncc(-c2nnc(C(F)F)o2)s1)c1cncc([C@@H](C)F)c1] | 1H NMR (400 MHz, CDCl3) δ 8.67 (d, J = 2.0 Hz, 1 H), 8.56 (s, 1 H), 8.39 (s, 1 H), 7.83 (s, 1 H), 6.91 (t, J = 52.0 Hz, 1 H), 5.78-5.62 (m, 1 H), 5.30 (s, 2 H), 3.21 (q, J = 7.2 Hz, 2 H), 1.74-1.62 (m, 3 H), 1.46 (t, J = 7.6 Hz, 3 H). LCMS RT = 1.503 min, m/z = 447.8 | 0.027 |
| | N-({5-[5-(difluoromethyl)-1,3,4- | | |
| | oxadiazol-2-yl]-1,3-thiazol-2- | | |
| | yl}methyl)-N-{5-[(1R)-1- | | |
| | fluoroethyl]pyridin-3-yl}ethane-1- | | |
| | sulfonamide | | |
| I-209 | 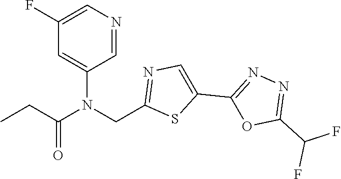 [SMILES: CCC(=O)N(Cc1ncc(-c2nnc(C(F)F)o2)s1)c1cncc(F)c1] | 1H NMR (400 MHz, CDCl3) δ 8.54 (d, J = 2.4 Hz, 1 H), 8.39- 8.35 (m, 2 H), 7.44-7.40 (m, 1 H), 6.92 (t, J = 51.6 Hz, 1 H), 5.19 (s, 2 H), 2.19 (q, J = 6.8 Hz, 2 H), 1.13 (t, J = 7.6 Hz, 3 H). LCMS RT = 1.755 min, m/z = 384.2 | 0.284 |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)propanamide | | |
| I-210 | 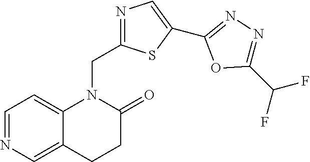 [SMILES: O=C1CCc2cnccc2N1Cc1ncc(-c2nnc(C(F)F)o2)s1] | 1H NMR (400 MHz, CDCl3) δ 8.49-8.37 (m, 3 H), 7.19 (d, J = 5.6 Hz, 1 H), 6.89 (t, J = 51.6 Hz, 1 H), 5.46 (s, 2 H), 3.10- 3.01 (m, 2 H), 2.91-2.80 (m, 2 H). LCMS RT = 0.971 min, m/z = 364.2 | 0.0487 |
| | 1-({5-[5-(difluoromethyl)-1,3,4- | | |
| | oxadiazol-2-yl]-1,3-thiazol-2- | | |
| | yl}methyl)-1,2,3,4-tetrahydro-1,6- | | |
| | naphthyridin-2-one | | |
| I-211 | 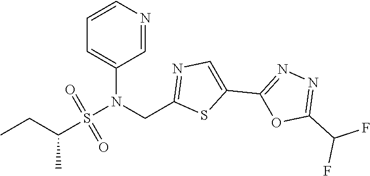 [SMILES: CC[C@@H](C)S(=O)(=O)N(Cc1ncc(-c2nnc(C(F)F)o2)s1)c1cccnc1] | 1H NMR (400 MHz, CDCl3) δ 8.70 (s, 1 H), 8.57-8.55 (m, 1 H), 8.35 (s, 1 H), 7.87-7.82 (m, 1 H), 7.38-7.32 (m, 1 H), 6.91 (t, J = 51.6 Hz, 1 H), 5.38- 5.20 (m, 2 H), 3.14-3.06 (m, 1 H), 2.13-2.05 (m, 1 H), 1.72-1.65 (m, 1 H), 1.45 (d, J = 6.8 Hz, 3 H), 1.04 (t, J = 7.6 Hz, 3 H). LCMS RT = 0.563 min, m/z = 430.1 | 0.0137 |
| | (2R)-N-({5-[5-(difluoromethyl)- | | |
| | 1,3,4-oxadiazol-2-yl]-1,3-thiazol- | | |
| | 2-yl}methyl)-N-(pyridin-3- | | |
| | yl)butane-2-sulfonamide | | |
| I-212 | 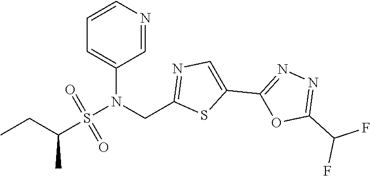 [SMILES: CC[C@H](C)S(=O)(=O)N(Cc1ncc(-c2nnc(C(F)F)o2)s1)c1cccnc1] | 1H NMR (400 MHz, CDCl3) δ 8.70 (s, 1 H), 8.58-8.55 (m, 1 H), 8.35 (s, 1 H), 7.89-7.86 (m, 1 H), 7.34 (q, J = 4.8 Hz, 1 H), 6.91 (t, J = 51.6 Hz, 1 H), 5.39-5.23 (m, 2 H), 3.14- 3.06 (m, 1 H), 2.14-2.04 (m, 1 H), 1.74-1.65 (m, 1 H), 1.45 (d, J = 6.8 Hz, 3 H), 1.04 (t, J = 7.6 Hz, 3 H). LCMS RT = 0.563 min, m/z = 430.1 | 0.0105 |
| | (2S)-N-({5-[5-(difluoromethyl)- | | |
| | 1,3,4-oxadiazol-2-yl]-1,3-thiazol- | | |
| | 2-yl}methyl)-N-(pyridin-3- | | |
| | yl)butane-2-sulfonamide | | |
| I-213 | 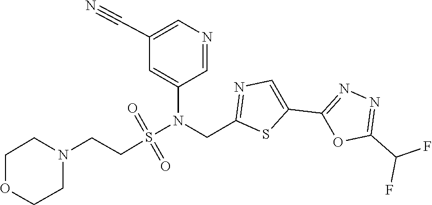 [SMILES: N#Cc1cncc(N(Cc2ncc(-c3nnc(C(F)F)o3)s2)S(=O)(=O)CCN2CCOCC2)c1] | 1H NMR (400 MHz, CDCl3) δ 8.96 (d, J = 2.4 Hz, 1 H), 8.82 (s, 1 H), 8.41 (s, 1 H), 8.19 (s, 1 H), 6.92 (t, J = 51.6 Hz, 1 H), 5.33 (s, 2 H), 3.74 (t, J = 4.8 Hz, 4 H), 3.38 (t, J = 6.8 Hz, 2 H), 2.89 (t, J = 7.2 Hz, 2 H), 2.50 (t, J = 4.8 Hz, 4 H). LCMS RT = 1.295 min, m/z = 512.2 | 0.0902 |
| | N-(5-cyanopyridin-3-yl)-N-({5-[5- | | |
| | (difluoromethyl)-1,3,4-oxadiazol- | | |
| | 2-yl]-1,3-thiazol-2-yl}methyl)-2- | | |
| | (morpholin-4-yl)ethane-1- | | |
| | sulfonamide | | |
| I-214 | 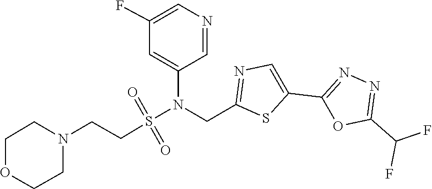 [SMILES: O=S(=O)(CCN1CCOCC1)N(Cc1ncc(-c2nnc(C(F)F)o2)s1)c1cncc(F)c1] | 1H NMR (400 MHz, CDCl3) δ ppm 8.59 (s, 1 H), 8.47 (d, J = 2.4 Hz, 1 H), 8.40 (s, 1 H), 7.71-7.67 (m, 1 H), 6.93 (t, J = 51.6 Hz, 1 H), 5.33 (s, 2 H), 3.78-3.72 (m, 4 H), 3.40 (t, J = 6.8 Hz, 2 H), 2.91 (t, J = 6.8 Hz, 2 H), 2.53-2.51 (m, 4 H). LCMS RT = 0.908 min, m/z = 505.2 | 0.0154 |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)-2-(morpholin-4-yl)ethane-1- | | |
| | sulfonamide | | |
| I-215 | 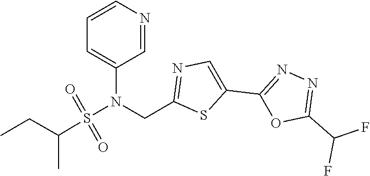 [SMILES: CCC(C)S(=O)(=O)N(Cc1ncc(-c2nnc(C(F)F)o2)s1)c1cccnc1] | 1H NMR (400 MHz, CDCl3) δ 8.72 (d, J = 2.8 Hz, 1 H), 8.58- 8.56 (m, 1 H), 8.35 (s, 1 H), 7.89-7.86 (m, 1 H), 7.36 (q, J = 4.8 Hz, 1 H), 6.91 (t, J = 51.6 Hz, 1 H), 5.37-5.26 (m, 2 H), 3.14-3.05 (m, 1 H), 2.12- 2.06 (m, 1 H), 1.75-1.69 (m, 1 H), 1.45 (d, J = 6.8 Hz, 3 H), 1.04 (t, J = 7.6 Hz, 3 H). LCMS RT = 0.560 min, m/z = 430.1 | 0.00675 |
| | N-({5-[5-(difluoromethyl)-1,3,4- | | |
| | oxadiazol-2-yl]-1,3-thiazol-2- | | |
| | yl}methyl)-N-(pyridin-3- | | |
| | yl)butane-2-sulfonamide | | |
| I-216 | 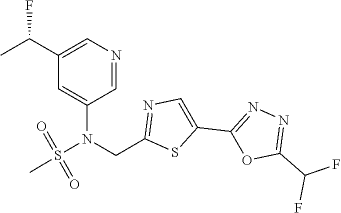 [SMILES: C[C@H](F)c1cncc(N(Cc2ncc(-c3nnc(C(F)F)o3)s2)S(C)(=O)=O)c1] | 1H NMR (400 MHz, CDCl3) δ 8.68 (s, 1 H), 8.58 (s, 1 H), 8.42 (s, 1 H), 7.84 (s, 1 H), 6.92 (t, J = 51.6 Hz, 1 H), 5.80- 5.61 (m, 1 H), 5.29 (s, 2 H), 3.11 (s, 3 H), 1.75-1.55 (m, 3 H). LCMS RT = 0.550 min, m/z = 434.1 | 0.0119 |
| | N-({5-[5-(difluoromethyl)-1,3,4- | | |
| | oxadiazol-2-yl]-1,3-thiazol-2- | | |
| | yl}methyl)-N-{5-[(1S)-1- | | |
| | fluoroethyl]pyridin-3- | | |
| | yl}methanesulfonamide | | |
| I-217 | 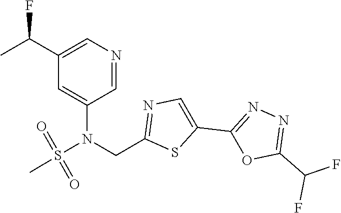 [SMILES: C[C@@H](F)c1cncc(N(Cc2ncc(-c3nnc(C(F)F)o3)s2)S(C)(=O)=O)c1] | 1H NMR (400 MHz, CDCl3) δ 8.70 (s, 1 H), 8.58 (s, 1 H), 8.42 (s, 1 H), 7.86 (s, 1 H), 6.92 (t, J = 51.6 Hz, 1 H), 5.81- 5.60 (m, 1 H), 5.29 (s, 2 H), 3.11 (s, 3 H), 1.75-1.55 (m, 3 H). LCMS RT = 0.551 min, m/z = 434.1 | 0.0236 |
| | N-({5-[5-(difluoromethyl)-1,3,4- | | |
| | oxadiazol-2-yl]-1,3-thiazol-2- | | |
| | yl}methyl)-N-{5-[(1R)-1- | | |
| | fluoroethyl]pyridin-3- | | |
| | yl}methanesulfonamide | | |
| I-218 | 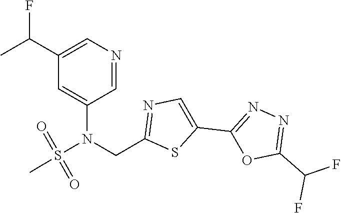 [SMILES: CC(F)c1cncc(N(Cc2ncc(-c3nnc(C(F)F)o3)s2)S(C)(=O)=O)c1] | 1H NMR (400 MHz, CDCl3) δ 8.68 (d, J = 2.4 Hz, 1 H), 8.57 (s, 1 H), 8.41 (s, 1 H), 7.84 (s, 1 H), 6.91 (t, J = 51.2 Hz, 1 H), 5.79-5.62 (m, 1 H), 5.28 (s, 2 H), 3.11 (s, 3 H), 1.75-1.55 (m, 3 H). LCMS RT = 0.545 min, m/z = 434.1 | 0.0369 |
| | N-({5-[5-(difluoromethyl)-1,3,4- | | |
| | oxadiazol-2-yl]-1,3-thiazol-2- | | |
| | yl}methyl)-N-[5-(1- | | |
| | fluoroethyl)pyridin-3- | | |
| | yl]methanesulfonamide | | |
| I-219 | 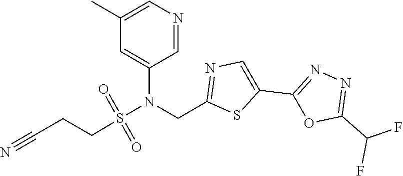 [SMILES: Cc1cncc(N(Cc2ncc(-c3nnc(C(F)F)o3)s2)S(=O)(=O)CCC#N)c1] | 1H NMR (400 MHz, CDCl3) δ 8.52 (d, J = 2.0 Hz, 1 H), 8.47 (s, 1 H), 8.45 (s, 1 H), 7.65 (s, 1 H), 6.92 (t, J = 51.6 Hz, 1 H), 5.28 (s, 2 H), 3.53 (t, J = 7.6 Hz, 2 H), 2.98 (t, J = 7.2 Hz, 2 H), 2.39 (s, 3 H). LCMS RT = 0.507 min, m/z = 441.1 | 0.0202 |
| | 2-cyano-N-({5-[5- | | |
| | (difluoromethyl)-1,3,4-oxadiazol- | | |
| | 2-yl]-1,3-thiazol-2-yl}methyl)-N- | | |
| | (5-methylpyridin-3-yl)ethane-1- | | |
| | sulfonamide | | |
| I-220 | 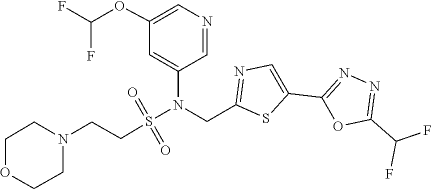 [SMILES: O=S(=O)(CCN1CCOCC1)N(Cc1ncc(-c2nnc(C(F)F)o2)s1)c1cncc(OC(F)F)c1] | 1H NMR (400 MHz, CDCl3) δ 8.61 (s, 1 H), 8.46 (s, 1 H), 8.39 (s, 1 H), 7.70 (s, 1 H), 6.91 (t, J = 51.6 Hz, 1 H), 6.59 (t, J = 72.0 Hz, 1 H), 5.31 (s, 2 H), 3.77-3.69 (m, 4 H), 3.38 (t, J = 6.8 Hz, 2 H), 2.89 (t, J = 7.2 Hz, 2 H), 2.54-2.46 (m, 4 H) LCMS RT = 1.504 min, m/z = 553.2 | 0.0184 |
| | N-[5-(difluoromethoxy)pyridin-3- | | |
| | yl]-N-({5-[5-(difluoromethyl)- | | |
| | 1,3,4-oxadiazol-2-yl]-1,3-thiazol- | | |
| | 2-yl}methyl)-2-(morpholin-4- | | |
| | yl)ethane-1-sulfonamide | | |
| I-221 | 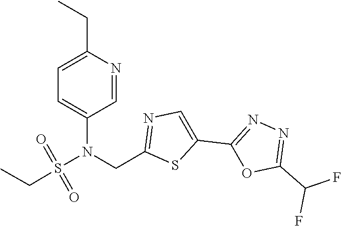 [SMILES: CCc1ccc(N(Cc2ncc(-c3nnc(C(F)F)o3)s2)S(=O)(=O)CC)cn1] | 1H NMR (400 MHz, CDCl3) δ 8.58 (d, J = 2.4 Hz, 1 H), 8.37 (s, 1 H), 7.75-7.70 (m, 1 H), 7.22 (d, J = 8.4 Hz, 1 H), 6.91 (t, J = 51.6 Hz, 1 H), 5.7 (s, 2 H), 3.20 (q, J = 7.6 Hz , 2 H), 2.83 (q, J = 7.6 Hz , 2 H), 1.46 (t, J = 7.2 Hz, 3 H), 1.30 (t, J = 7.6 Hz, 3 H). LCMS RT = 1.134 min, m/z = 430.2 | 0.033 |
| | N-({5-[5-(difluoromethyl)-1,3,4- | | |
| | oxadiazol-2-yl]-1,3-thiazol-2- | | |
| | yl}methyl)-N-(6-ethylpyridin-3- | | |
| | yl)ethane-1-sulfonamide | | |
| I-222 | 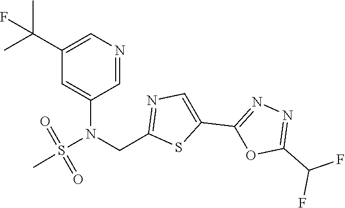 [SMILES: CC(C)(F)c1cncc(N(Cc2ncc(-c3nnc(C(F)F)o3)s2)S(C)(=O)=O)c1] | 1H NMR (400 MHz, CDCl3) δ 8.64 (d, J = 2.0 Hz, 1 H), 8.60 (s, 1 H), 8.42 (s, 1 H), 7.86 (t, J = 2.0 Hz, 1 H), 6.92 (t, J = 51.6 Hz, 1 H), 5.28 (s, 2 H), 3.11 (s, 3 H), 1.75 (s, 3 H), 1.69 (s, 3 H). LCMS RT = 1.499 min, m/z = 447.9 | 0.0376 |
| | N-({5-[5-(difluoromethyl)-1,3,4- | | |
| | oxadiazol-2-yl]-1,3-thiazol-2- | | |
| | yl}methyl)-N-[5-(2-fluoropropan- | | |
| | 2-yl)pyridin-3- | | |
| | yl]methanesulfonamide | | |
| I-223 | 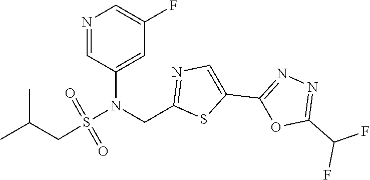 [SMILES: CC(C)CS(=O)(=O)N(Cc1ncc(-c2nnc(C(F)F)o2)s1)c1cncc(F)c1] | 1H NMR (400 MHz, CDCl3) δ 8.58 (s, 1 H), 8.47 (d, J = 2.8 Hz, 1 H), 8.40 (s, 1 H), 7.68- 7.62 (m, 1 H), 6.91 (t, J = 51.6 Hz, 1 H), 5.29 (s, 2 H), 3.05 (d, J = 2.0 Hz, 2 H), 2.40-2.30 (m, 1 H), 1.12 (d, J = 6.8 Hz, 6 H). LCMS RT = 1.790 min, m/z = 447.9 | 0.00563 |
| | N-({5-[5-(difluoromethyl)-1,3,4- | | |
| | oxadiazol-2-yl]-1,3-thiazol-2- | | |
| | yl}methyl)-N-(5-fluoropyridin-3- | | |
| | yl)-2-methylpropane-1- | | |
| | sulfonamide | | |
| I-224 | 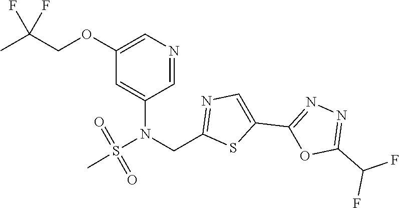 [SMILES: CC(F)(F)COc1cncc(N(Cc2ncc(-c3nnc(C(F)F)o3)s2)S(C)(=O)=O)c1] | 1H NMR (400 MHz, CDCl3) δ 8.42 (s, 1 H), 8.39-8.33 (m, 2 H), 7.48-7.41 (m, 1 H), 6.92 (t, J = 51.6 Hz, 1 H), 5.28 (s, 2 H), 4.18 (t, J = 11.2 Hz, 2 H), 3.11 (s, 3 H), 1.79 (t, J = 18.8 Hz, 3 H). LCMS RT = 1.492 min, m/z = 482.2 | 0.0204 |
| | N-({5-[5-(difluoromethyl)-1,3,4- | | |
| | oxadiazol-2-yl]-1,3-thiazol-2- | | |
| | yl}methyl)-N-[5-(2,2- | | |
| | difluoropropoxy)pyridin-3- | | |
| | yl]methanesulfonamide | | |
| I-225 | 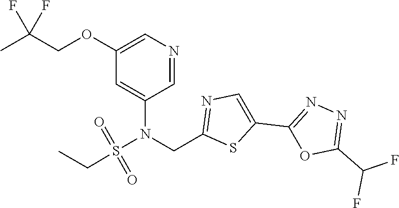 [SMILES: CCS(=O)(=O)N(Cc1ncc(-c2nnc(C(F)F)o2)s1)c1cncc(OCC(C)(F)F)c1] | 1H NMR (400 MHz, CDCl3) δ 8.42-8.36 (m, 2 H), 8.33 (s, 1 H), 7.45 (s, 1 H), 6.91 (t, J = 51.6 Hz, 1 H), 5.30 (s, 2 H), 4.17 (t, J = 11.2 Hz, 2 H), 3.21 (q, J = 7.6 Hz, 2 H), 1.79 (t, J = 18.8 Hz, 3 H), 1.46 (t, J = 7.6 Hz, 3 H). LCMS RT = 1.559 min, m/z = 496.2 | 0.00601 |
| | N-({5-[5-(difluoromethyl)-1,3,4- | | |
| | oxadiazol-2-yl]-1,3-thiazol-2- | | |
| | yl}methyl)-N-[5-(2,2- | | |
| | difluoropropoxy)pyridin-3- | | |
| | ylJethane-1-sulfonamide | | |
| I-226 | 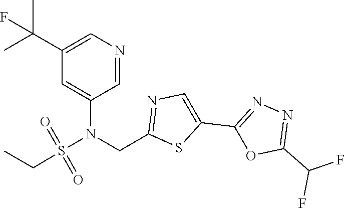 [SMILES: CCS(=O)(=O)N(Cc1ncc(-c2nnc(C(F)F)o2)s1)c1cncc(C(C)(C)F)c1] | 1H NMR (400 MHz, CDCl3) δ 8.66 (d, J = 2.4 Hz, 1 H), 8.59 (d, J = 1.2 Hz, 1 H), 8.40 (s, 1 H), 7.91-7.86 (m, 1 H), 6.91 (t, J = 51.6 Hz, 1 H), 5.30 (s, 2 H), 3.21 (q, J = 6.8 Hz, 2 H), 1.75 (s, 3 H), 1.69 (s, 3 H), 1.47 (t, J = 6.4 Hz, 3 H). LCMS RT = 1.510 min, m/z = 462.2 | 0.0319 |
| | N-({5-[5-(difluoromethyl)-1,3,4- | | |
| | oxadiazol-2-yl]-1,3-thiazol-2- | | |
| | yl}methyl)-N-[5-(2-fluoropropan- | | |
| | 2-yl)pyridin-3-yl]ethane-1- | | |
| | sulfonamide | | |
| I-227 | 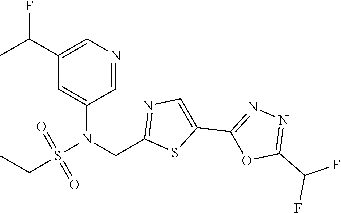 [SMILES: CCS(=O)(=O)N(Cc1ncc(-c2nnc(C(F)F)o2)s1)c1cncc(C(C)F)c1] | 1H NMR (400 MHz, CDCl3) δ 8.67 (d, J = 2.4 Hz, 1 H), 8.56 (s, 1 H), 8.39 (s, 1 H), 7.83 (s, 1 H), 6.91 (t, J = 51.6 Hz, 1 H), 5.79-5.62 (m, 1 H), 5.30 (s, 2 H), 3.21 (q, J = 7.2 Hz, 2 H), 1.74-1.56 (m, 3 H), 1.47 (t, J = 7.2 Hz, 3 H). LCMS RT = 1.518 min, m/z = 447.9 | 0.0123 |
| | N-({5-[5-(difluoromethyl)-1,3,4- | | |
| | oxadiazol-2-yl]-1,3-thiazol-2- | | |
| | yl}methyl)-N-[5-(1- | | |
| | fluoroethyl)pyridin-3-yl]ethane-1- | | |
| | sulfonamide | | |
| I-228 | 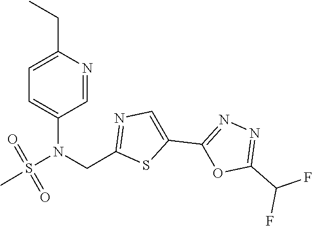 [SMILES: CCc1ccc(N(Cc2ncc(-c3nnc(C(F)F)o3)s2)S(C)(=O)=O)cn1] | 1H NMR (400 MHz, CD3OD) δ 8.54 (d, J = 2.4 Hz, 1 H), 8.39 (s, 1 H), 7.93-7.88 (m, 1 H), 7.35 (d, J = 8.4 Hz, 1 H), 7.17 (t, J = 51.6 Hz, 1 H), 5.31 (s, 2 H), 3.12 (s, 3 H), 2.78 (q, J = 7.6 Hz, 2 H), 1.24 (t, J = 7.6 Hz, 3 H). LCMS RT = 0.981 min, m/z = 416.2 | 0.0435 |
| | N-({5-[5-(difluoromethyl)-1,3,4- | | |
| | oxadiazol-2-yl]-1,3-thiazol-2- | | |
| | yl}methyl)-N-(6-ethylpyridin-3- | | |
| | yl)methanesulfonamide | | |
| I-229 | 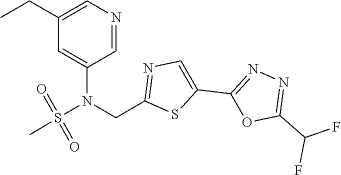 [SMILES: CCc1cncc(N(Cc2ncc(-c3nnc(C(F)F)o3)s2)S(C)(=O)=O)c1] | 1H NMR (400 MHz, CD3OD) δ 8.51 (s, 1 H), 8.42 (s, 1 H), 8.40 (s, 1 H), 7.89 (s, 1 H), 7.18 (t, J = 51.6 Hz, 1 H), 5.35 (s, 2 H), 3.14 (s, 3 H), 2.69 (q, J = 7.2 Hz, 2 H), 1.25 (t, J = 7.6 Hz, 3 H). LCMS RT = 1.099 min, m/z = 416.2 | 0.0248 |
| | N-({5-[5-(difluoromethyl)-1,3,4- | | |
| | oxadiazol-2-yl]-1,3-thiazol-2- | | |
| | yl}methyl)-N-(5-ethylpyridin-3- | | |
| | yl)methanesulfonamide | | |
| I-230 | 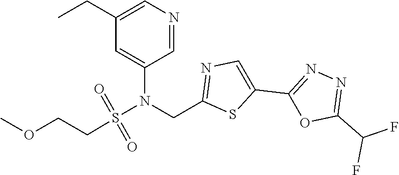 [SMILES: CCc1cncc(N(Cc2ncc(-c3nnc(C(F)F)o3)s2)S(=O)(=O)CCOC)c1] | 1H NMR (400 MHz, CD3OD) δ 8.50 (d, J = 2.4 Hz, 1 H), 8.38 (s, 1 H), 8.35 (s, 1 H), 7.88 (s, 1 H), 7.18 (t, J = 51.6 Hz, 1 H), 5.31 (s, 2 H), 3.82 (t, J = 5.6 Hz, 2 H), 3.48 (t, J = 5.2 Hz, 2 H), 3.43 (s, 3 H), 2.69 (q, J = 5.2 Hz, 2 H), 1.24 (t, J = 7.6 Hz, 3 H). LCMS RT = 1.752 min, m/z = 460.2 | 0.033 |
| | N-({5-[5-(difluoromethyl)-1,3,4- | | |
| | oxadiazol-2-yl]-1,3-thiazol-2- | | |
| | yl}methyl)-N-(5-ethylpyridin-3- | | |
| | yl)-2-methoxyethane-1- | | |
| | sulfonamide | | |
| I-231 | 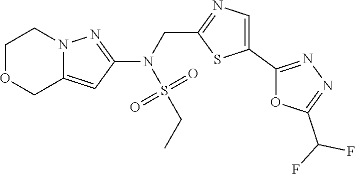 [SMILES: CCS(=O)(=O)N(Cc1ncc(-c2nnc(C(F)F)o2)s1)c1cc2n(n1)CCOC2] | 1H NMR (400 MHz, CDCl3) δ 8.39 (s, 1 H), 6.90 (t, J = 51.6 Hz, 1 H), 6.12 (s, 1 H), 5.38 (s, 2 H), 4.77 (s, 2 H), 4.12-4.06 (m, 4 H), 3.27 (q, J = 7.2 Hz, 2 H), 1.42 (t, J = 7.6 Hz, 3 H). LCMS RT = 0.552 min, m/z = 447.1 | 0.0924 |
| | N-({5-[5-(difluoromethyl)-1,3,4- | | |
| | oxadiazol-2-yl]-1,3-thiazol-2- | | |
| | yl}methyl)-N-{4H,6H,7H- | | |
| | pyrazolo[3,2-c][1,4]oxazin-2- | | |
| | yl}ethane-1-sulfonamide | | |
| I-232 | 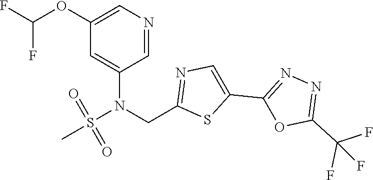 [SMILES: CS(=O)(=O)N(Cc1ncc(-c2nnc(C(F)(F)F)o2)s1)c1cncc(OC(F)F)c1] | 1H NMR (400 MHz, CDCl3) δ 8.63 (d, J = 2.0 Hz, 1 H), 8.51 (d, J = 2.4 Hz, 1 H), 8.46 (s, 1 H), 7.70 (s, 1 H), 6.61 (t, J = 71.6 Hz, 1 H), 5.30 (s, 2 H), 3.13 (s, 3 H). LCMS RT = 1.766 min, m/z = 471.8 | 0.0342 |
| | N-[5-(difluoromethoxy)pyridin-3- | | |
| | yl]-N-({5-[5-(trifluoromethyl)- | | |
| | 1,3,4-oxadiazol-2-yl]-1,3-thiazol- | | |
| | 2-yl}methyl)methanesulfonamide | | |
| I-233 | 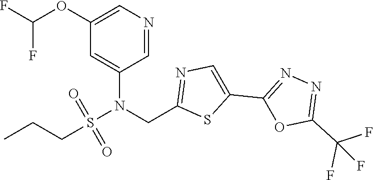 [SMILES: CCCS(=O)(=O)N(Cc1ncc(-c2nnc(C(F)(F)F)o2)s1)c1cncc(OC(F)F)c1] | 1H NMR (400 MHz, CDCl3) δ 8.59 (d, J = 2.0 Hz, 1 H), 8.47 (d, J = 2.4 Hz, 1 H), 8.42 (s, 1 H), 7.67 (t, J = 2.4 Hz, 1 H), 6.59 (t, J = 72.0 Hz, 1 H), 5.29 (s, 2 H), 3.19-3.11 (m, 2 H), 1.97-1.88 (m, 2 H), 1.08 (t, J = 7.6 Hz, 3 H). LCMS RT = 2.013 min, m/z = 499.9 | 0.0606 |
| | N-[5-(difluoromethoxy)pyridin-3- | | |
| | yl]-N-({5-[5-(trifluoromethyl)- | | |
| | 1,3,4-oxadiazol-2-yl]-1,3-thiazol- | | |
| | 2-yl}methyl)propane-1- | | |
| | sulfonamide | | |
| I-234 | 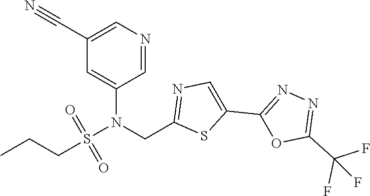 [SMILES: CCCS(=O)(=O)N(Cc1ncc(-c2nnc(C(F)(F)F)o2)s1)c1cncc(C#N)c1] | 1H NMR (400 MHz, CDCl3) δ 8.95 (d, J = 2.4 Hz, 1 H), 8.82 (s, 1 H), 8.44 (s, 1 H), 8.16 (d, J = 2.4 Hz, 1 H), 5.29 (s, 2 H), 3.18-3.11 (m, 2 H), 2.00- 1.80 (m, 2 H), 1.09 (t, J = 7.2 Hz, 3 H). LCMS RT = 1.811 min, m/z = 459.2 | 0.184 |
| | N-(5-cyanopyridin-3-yl)-N-({5-[5- | | |
| | (trifluoromethyl)-1,3,4-oxadiazol- | | |
| | 2-yl]-1,3-thiazol-2- | | |
| | yl}methyl)propane-1-sulfonamide | | |
| I-235 | 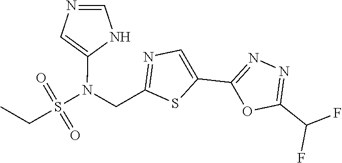 [SMILES: CCS(=O)(=O)N(Cc1ncc(-c2nnc(C(F)F)o2)s1)c1cnc[nH]1] | 1H NMR (400 MHz, CD3OD) δ 8.43 (s, 1 H), 7.99 (s, 1 H), 7.39-7.03 (m, 2 H), 5.30 (s, 2 H), 3.39-3.28 (m, 2 H), 1.40 (t, J = 7.2 Hz, 3 H). LCMS RT = 1.848 min, m/z = 390.8 | 0.323 |
| | N-({5-[5-(difluoromethyl)-1,3,4- | | |
| | oxadiazol-2-yl]-1,3-thiazol-2- | | |
| | yl}methyl)-N-(1H-imidazol-5- | | |
| | yl)ethane-1-sulfonamide | | |
| I-236 | 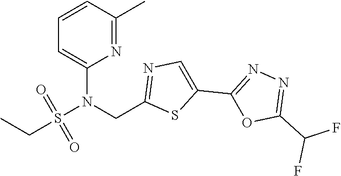 [SMILES: CCS(=O)(=O)N(Cc1ncc(-c2nnc(C(F)F)o2)s1)c1cccc(C)n1] | 1H NMR (400 MHz, CDCl3) δ 8.39 (s, 1 H), 7.61 (t, J = 8.0 Hz, 1 H), 7.45 (d, J = 8.4 Hz, 1 H), 7.01 (s, 1 H), 6.89 (t, J = 51.6 Hz, 1 H), 5.60 (s, 2 H), 3.27 (q, J = 7.6 Hz, 2 H), 2.52 (s, 3 H), 1.37 (t, J = 7.4 Hz, 3 H). LCMS RT = 1.704 min, m/z = 416.2 | 0.0592 |
| | N-({5-[5-(difluoromethyl)-1,3,4- | | |
| | oxadiazol-2-yl]-1,3-thiazol-2- | | |
| | yl}methyl)-N-(6-methylpyridin-2- | | |
| | yl)ethane-1-sulfonamide | | |
| I-237 | 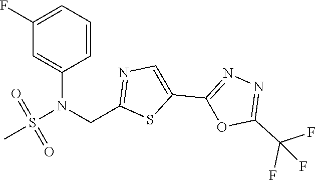 [SMILES: CS(=O)(=O)N(Cc1ncc(-c2nnc(C(F)(F)F)o2)s1)c1cccc(F)c1] | 1H NMR (400 MHz, CDCl3) δ 8.41 (s, 1 H), 7.44-7.36 (m, 1 H), 7.30-7.27 (m, 1 H), 7.24- 7.20 (m, 1 H), 7.12-7.05 (m, 1 H), 5.27 (s, 2 H), 3.07 (s, 3 H). LCMS RT = 1.912 min, m/z = 422.8 | 0.0992 |
| | N-(3-fluorophenyl)-N-({5-[5- | | |
| | (trifluoromethyl)-1,3,4-oxadiazol- | | |
| | 2-yl]-1,3-thiazol-2- | | |
| | yl}methyl)methanesulfonamide | | |
| I-238 | 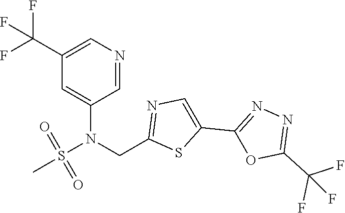 [SMILES: CS(=O)(=O)N(Cc1ncc(-c2nnc(C(F)(F)F)o2)s1)c1cncc(C(F)(F)F)c1] | 1H NMR (400 MHz, CDCl3) δ 8.95 (d, J = 2.0 Hz, 1 H), 8.87 (s, 1 H), 8.46 (s, 1 H), 8.12 (t, J = 2.0 Hz, 1 H), 5.31 (s, 2 H), 3.12 (s, 3 H). LCMS RT = 1.905 min, m/z = 473.8 | 0.256 |
| | N-({5-[5-(trifluoromethyl)-1,3,4- | | |
| | oxadiazol-2-yl]-1,3-thiazol-2- | | |
| | yl}methyl)-N-[5- | | |
| | (trifluoromethyl)pyridin-3- | | |
| | yl]methanesulfonamide | | |
| I-239 | 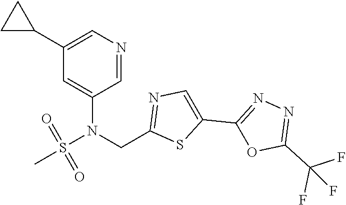 [SMILES: CS(=O)(=O)N(Cc1ncc(-c2nnc(C(F)(F)F)o2)s1)c1cncc(C2CC2)c1] | 1H NMR (400 MHz, CDCl3) δ 8.46 (s, 1 H), 8.42 (s, 1 H), 8.37 (s, 1 H), 7.45 (t, J = 2.4 Hz, 1 H), 5.26 (s, 2 H), 3.09 (s, 3 H), 2.00-1.90 (m, 1 H), 1.11-1.08 (m, 2 H), 0.78- 0.75 (m, 2 H). LCMS RT = 1.621 min, m/z = 445.9 | 0.085 |
| | N-(5-cyclopropylpyridin-3-yl)-N- | | |
| | ({5-[5-(trifluoromethyl)-1,3,4- | | |
| | oxadiazol-2-yl]-1,3-thiazol-2- | | |
| | yl}methyl)methanesulfonamide | | |
| I-240 | 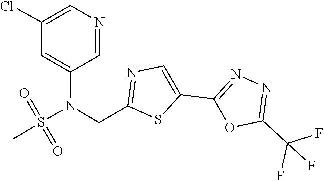 [SMILES: CS(=O)(=O)N(Cc1ncc(-c2nnc(C(F)(F)F)o2)s1)c1cncc(Cl)c1] | 1H NMR (400 MHz, CDCl3) δ 8.62 (s, 1 H), 8.57 (s, 1 H), 7.89 (s, 1 H), 5.28 (s, 2 H), 3.11 (s, 3 H). LCMS RT = 2.259 min, m/z = 440.1 | 0.062 |
| | N-(5-chloropyridin-3-yl)-N-({5- | | |
| | [5-(trifluoromethyl)-1,3,4- | | |
| | oxadiazol-2-yl]-1,3-thiazol-2- | | |
| | yl}methyl)methanesulfonamide | | |
| I-241 | 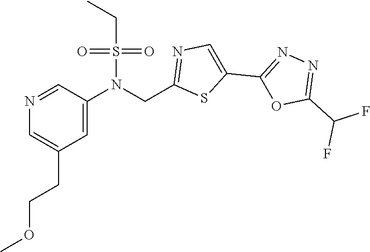 [SMILES: CCS(=O)(=O)N(Cc1ncc(-c2nnc(C(F)F)o2)s1)c1cncc(CCOC)c1] | 1H NMR (400 MHz, CDCl3) δ 8.50 (s, 1 H), 8.48 (s, 1 H), 7.80 (s, 1 H), 7.65 (s, 1 H), 6.92 (t, J = 51.6 Hz, 1 H), 5.80 (s, 2 H), 3.60 (t, J = 6.0 Hz, 2 H), 3.33 (s, 3 H), 3.08 (q, J = 7.2 Hz, 2 H), 2.85 (t, J = 6.0 Hz, 2 H), 1.40 (t, J = 7.6 Hz, 3 H). LCMS RT = 0.718 min, m/z = 460.2 | 0.994 |
| | N-({5-[5-(difluoromethyl)-1,3,4- | | |
| | oxadiazol-2-yl]-1,3-thiazol-2- | | |
| | yl}methyl)-N-[5-(2- | | |
| | methoxyethyl)pyridin-3-yl]ethane- | | |
| | 1-sulfonamide | | |
| I-242 | 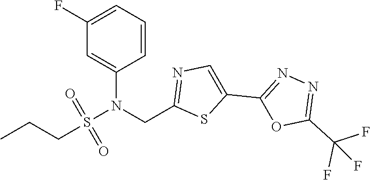 [SMILES: CCCS(=O)(=O)N(Cc1ncc(-c2nnc(C(F)(F)F)o2)s1)c1cccc(F)c1] | 1H NMR (400 MHz, CDCl3) δ 8.41 (s, 1 H), 7.43-7.37 (m, 1 H), 7.27-7.26 (m, 1 H), 7.25- 7.22 (m, 1 H), 7.10-7.05 (m, 1 H), 5.30 (s, 2 H), 3.15- 3.10 (m, 2 H), 1.97-1.89 (m, 2 H), 1.09 (t, J = 7.2 Hz, 3 H). LCMS RT = 2.207 min, m/z = 450.8 | 0.053 |
| | N-(3-fluorophenyl)-N-({5-[5- | | |
| | (trifluoromethyl)-1,3,4-oxadiazol- | | |
| | 2-yl]-1,3-thiazol-2- | | |
| | yl}methyl)propane-1-sulfonamide | | |
| I-243 | 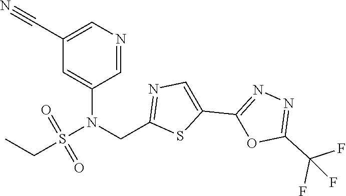 [SMILES: CCS(=O)(=O)N(Cc1ncc(-c2nnc(C(F)(F)F)o2)s1)c1cncc(C#N)c1] | 1H NMR (400 MHz, CDCl3) δ 8.96 (d, J = 2.4 Hz, 1 H), 8.82 (d, J = 2.0 Hz, 1 H), 8.44 (s, 1 H), 8.17 (t, J = 2.0 Hz, 1 H), 5.30 (s, 2 H), 3.21 (q, J = 7.2 Hz, 2 H), 1.45 (t, J = 7.2 Hz, 3 H). LCMS RT = 1.718 min, m/z = 444.8 | 0.129 |
| | N-(5-cyanopyridin-3-yl)-N-({5-[5- | | |
| | (trifluoromethyl)-1,3,4-oxadiazol- | | |
| | 2-yl]-1,3-thiazol-2- | | |
| | yl}methyl)ethane-1-sulfonamide | | |
| I-244 | 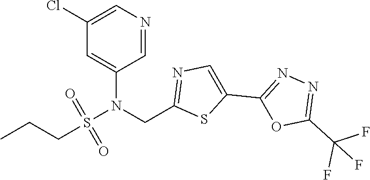 [SMILES: CCCS(=O)(=O)N(Cc1ncc(-c2nnc(C(F)(F)F)o2)s1)c1cncc(Cl)c1] | 1H NMR (400 MHz, CDCl3) δ 8.61 (d, J = 2.4 Hz, 1 H), 8.55 (d, J = 2.4 Hz, 1 H), 8.42 (s, 1 H), 7.88 (t, J = 2.0 Hz, 1 H), 5.28 (s, 2 H), 3.19-3.11 (m, 2 H), 1.97-1.82 (m, 2 H), 1.09 (t, J = 7.6 Hz, 3 H). LCMS RT = 2.027 min, m/z = 467.8 | 0.0872 |
| | N-(5-chloropyridin-3-yl)-N-({5- | | |
| | [5-(trifluoromethyl)-1,3,4- | | |
| | oxadiazol-2-yl]-1,3-thiazol-2- | | |
| | yl}methyl)propane-1-sulfonamide | | |
| I-245 | 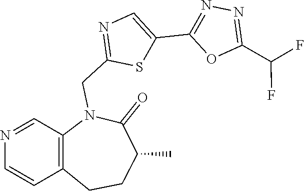 [SMILES: C[C@@H]1CCc2ccncc2N(Cc2ncc(-c3nnc(C(F)F)o3)s2)C1=O] | 1H NMR (400 MHz, CDCl3) δ 8.96 (s, 1 H), 8.52 (d, J = 5.2 Hz, 1 H), 8.38 (s, 1 H), 7.52 (d, J = 5.2 Hz, 1 H), 6.91 (t, J = 51.6 Hz, 1 H), 5.47-5.27 (m, 2 H), 3.09-3.04 (m, 1 H), 2.83-2.78 (m, 1 H), 2.51- 2.47 (m, 1 H), 2.26-2.21 (m, 1 H), 2.18-2.11 (m, 1 H), 1.16 (d, J = 6.4 Hz, 3 H) LCMS RT = 1.250 min, m/z = 392.3. | 1.42 |
| | (3R)-1-({5-[5-(difluoromethyl)- | | |
| | 1,3,4-oxadiazol-2-yl]-1,3-thiazol- | | |
| | 2-yl}methyl)-3-methyl- | | |
| | 1H,2H,3H,4H,5H-pyrido[3,4- | | |
| | b]azepin-2-one | | |
| I-246 | 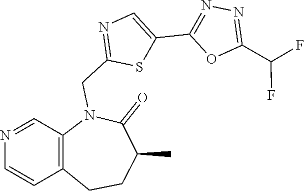 [SMILES: C[C@H]1CCc2ccncc2N(Cc2ncc(-c3nnc(C(F)F)o3)s2)C1=O] | 1H NMR (400 MHz, CDCl3) δ 8.91 (s, 1 H), 8.50 (d, J = 5.2 Hz, 1 H), 8.37 (s, 1 H), 7.47 (d, J = 5.2 Hz, 1 H), 6.91 (t, J = 51.6 Hz, 1 H), 5.46-5.28 (m, 2 H), 3.11-2.85 (m, 1 H), 2.84-2.71 (m, 1 H), 2.548- 2.45 (m, 1 H), 2.29-2.14 (m, 1 H), 2.18-2.07 (m, 1 H), 1.16 (d, J = 6.4 Hz, 3 H) LCMS RT = 1.251 min, m/z = 392.3. | 0.039 |
| | (3S)-1-({5-[5-(difluoromethyl)- | | |
| | 1,3,4-oxadiazol-2-yl]-1,3-thiazol- | | |
| | 2-yl}methyl)-3-methyl- | | |
| | 1H,2H,3H,4H,5H-pyrido[3,4- | | |
| | b]azepin-2-one | | |
| I-247 | 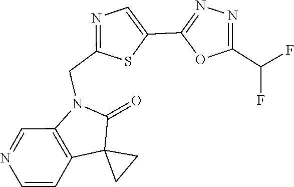 [SMILES: O=C1N(Cc2ncc(-c3nnc(C(F)F)o3)s2)c2cnccc2C12CC2] | 1H NMR (400 MHz, CDCl3) δ 8.47 (s, 1 H), 8.44-8.40 (m, 2 H), 7.07 (d, J = 5.2 Hz, 1 H), 6.90 (t, J = 51.6 Hz, 1 H), 5.43 (s, 2 H), 2.17-2.10 (m, 2 H), 1.93-1.86 (m, 2 H) LCMS RT = 0.970 min, m/z = 376.2. | 0.158 |
| | 1′-({5-[5-(difluoromethyl)-1,3,4- | | |
| | oxadiazol-2-yl]-1,3-thiazol-2- | | |
| | yl}methyl)-1′,2′- | | |
| | dihydrospiro[cyclopropane-1,3′- | | |
| | pyrrolo[2,3-c]pyridin]-2′-one | | |
| I-248 | 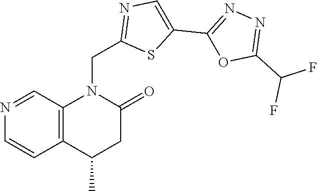 [SMILES: C[C@H]1CC(=O)N(Cc2ncc(-c3nnc(C(F)F)o3)s2)c2cnccc21] | 1H NMR (400 MHz, CDCl3) δ 8.54 (s, 1 H), 8.41 (s, 1 H), 8.35 (d, J = 4.8 Hz, 1 H), 7.19 (d, J = 4.8 Hz, 1 H), 6.89 (t, J = 51.6 Hz, 1 H), 5.62-5.46 (m, 2 H), 3.23-3.16 (m, 1 H), 2.92-2.84 (m, 1 H), 2.66- 2.55 (m, 1 H), 1.38 (d, J = 6.8 Hz, 3 H) LCMS RT =1.098 min, m/z = 378.3. | 0.034 |
| | (4S)-1-({5-[5-(difluoromethyl)- | | |
| | 1,3,4-oxadiazol-2-yl]-1,3-thiazol- | | |
| | 2-yl}methyl)-4-methyl-1,2,3,4- | | |
| | tetrahydro-1,7-naphthyridin-2-one | | |
| I-249 | 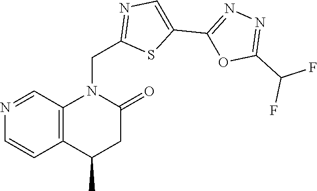 [SMILES: C[C@@H]1CC(=O)N(Cc2ncc(-c3nnc(C(F)F)o3)s2)c2cnccc21] | 1H NMR (400 MHz, CDCl3) δ 8.53 (s, 1 H), 8.40 (s, 1 H), 8.34 (d, J = 4.8 Hz, 1 H), 7.18 (d, J = 4.8 Hz, 1 H), 6.90 (t, J = 51.6 Hz, 1 H), 5.60-5.46 (m, 2 H), 3.23-3.15 (m, 1 H), 2.90-2.81 (m, 1 H), 2.64- 2.55 (m, 1 H), 1.38 (d, J = 6.8 Hz, 3 H) LCMS RT = 1.028 min, m/z = 378.2. | 0.089 |
| | (4R)-1-({5-[5-(difluoromethyl)- | | |
| | 1,3,4-oxadiazol-2-yl]-1,3-thiazol- | | |
| | 2-yl}methyl)-4-methyl-1,2,3,4- | | |
| | tetrahydro-1,7-naphthyridin-2-one | | |
| I-250 | 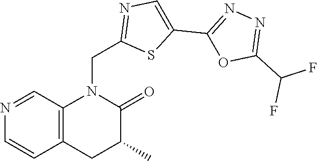 [SMILES: C[C@@H]1Cc2ccncc2N(Cc2ncc(-c3nnc(C(F)F)o3)s2)C1=O] | 1H NMR (400 MHz, CDCl3) δ 8.54 (s, 1 H), 8.41 (s, 1 H), 8.31 (d, J = 3.6 Hz, 1 H), 7.18 (d, J = 4.4 Hz, 1 H), 6.89 (t, J = 51.6 Hz, 1 H), 5.62-5.36 (m, 2 H), 3.09-3.02 (m, 1 H), 2.89-2.79 (m, 2 H), 1.35 (d, J = 6.8 Hz, 3 H). LCMS RT = 0.684 min, m/z = 378.2. | 0.041 |
| | (3R)-1-({5-[5-(difluoromethyl)- | | |
| | 1,3,4-oxadiazol-2-yl]-1,3-thiazol- | | |
| | 2-yl}methyl)-3-methyl-1,2,3,4- | | |
| | tetrahydro-1,7-naphthyridin-2-one | | |
| I-251 | 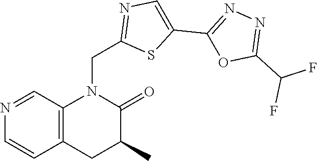 [SMILES: C[C@H]1Cc2ccncc2N(Cc2ncc(-c3nnc(C(F)F)o3)s2)C1=O] | 1H NMR (400 MHz, CDCl3) δ 8.52 (s, 1 H), 8.41 (s, 1 H), 8.30 (d, J = 4.4 Hz, 1 H), 7.17 (d, J = 4.8 Hz, 1 H), 6.89 (t, J = 51.6 Hz, 1 H), 5.62-5.36 (m, 2 H), 3.09-3.02 (m, 1 H), 2.89-2.79 (m, 2 H), 1.34 (d, J = 6.8 Hz, 3 H). LCMS RT = 0.521 min, m/z = 378.2. | 0.034 |
| | (3S)-1-({5-[5-(difluoromethyl)- | | |
| | 1,3,4-oxadiazol-2-yl]-1,3-thiazol- | | |
| | 2-yl}methyl)-3-methyl-1,2,3,4- | | |
| | tetrahydro-1,7-naphthyridin-2-one | | |
| I-252 | 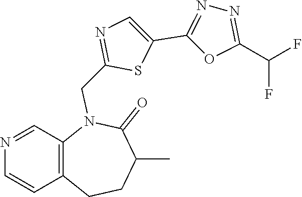 [SMILES: CC1CCc2ccncc2N(Cc2ncc(-c3nnc(C(F)F)o3)s2)C1=O] | 1H NMR (400 MHz, CDCl3) δ 8.77 (s, 1 H), 8.46 (d, J = 4.8 Hz, 1 H), 8.37 (s, 1 H), 7.30 (d, J = 4.4 Hz, 1 H), 6.90 (t, J = 51.6 Hz, 1 H), 5.37 (s, 2 H), 2.99-2.90 (m, 1 H), 2.70- 2.66 (m, 1 H), 2.55-2.49 (m, 1 H), 2.23-2.14 (m, 1 H), 2.18-2.04 (m, 1 H), 1.16 (d, J = 6.4 Hz, 3 H). LCMS RT = 1.229 min, m/z = 392.3. | 0.073 |
| | 1-({5-[5-(difluoromethyl)-1,3,4- | | |
| | oxadiazol-2-yl]-1,3-thiazol-2- | | |
| | yl}methyl)-3-methyl- | | |
| | 1H,2H,3H,4H,5H-pyrido[3,4- | | |
| | b]azepin-2-one | | |
| I-253 | 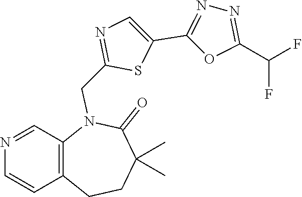 [SMILES: CC1(C)CCc2ccncc2N(Cc2ncc(-c3nnc(C(F)F)o3)s2)C1=O] | 1H NMR (400 MHz, CDCl3) δ 8.84 (s, 1 H), 8.47 (s, 1 H), 8.40 (s, 1 H), 7.42 (s, 1 H), 6.91 (t, J = 51.6 Hz, 1 H), 5.37 (s, 2 H), 2.91 (t, J = 6.0 Hz, 2 H), 2.18 (t, J = 6.4 Hz, 2 H), 1.04 (s, 6 H) LCMS RT = 1.946 min, m/z = 405.9. | 0.035 |
| | 1-({5-[5-(difluoromethyl)-1,3,4- | | |
| | oxadiazol-2-yl]-1,3-thiazol-2- | | |
| | yl}methyl)-3,3-dimethyl- | | |
| | 1H,2H,3H,4H,5H-pyrido[3,4- | | |
| | b]azepin-2-one | | |
| I-254 | 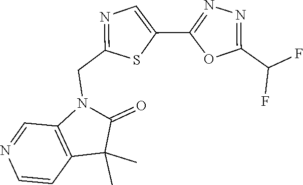 [SMILES: CC1(C)C(=O)N(Cc2ncc(-c3nnc(C(F)F)o3)s2)c2cnccc21] | 1H NMR (400 MHz, CDCl3) δ 8.45-8.43 (m, 2 H), 8.29 (s, 1 H), 7.24 (d, J = 4.8 Hz, 1 H), 6.89 (t, J = 51.6 Hz, 1 H), 5.33 (s, 2 H), 1.49 (s, 6 H) LCMS RT = 0.408 min, m/z = 378.1. | 0.149 |
| | 1-({5-[5-(difluoromethyl)-1,3,4- | | |
| | oxadiazol-2-yl]-1,3-thiazol-2- | | |
| | yl}methyl)-3,3-dimethyl- | | |
| | 1H,2H,3H-pyrrolo[2,3-c]pyridin- | | |
| | 2-one | | |
| 1-255 | 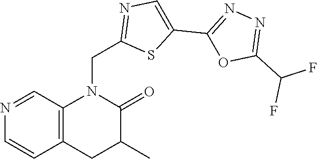 [SMILES: CC1Cc2ccncc2N(Cc2ncc(-c3nnc(C(F)F)o3)s2)C1=O] | 1H NMR (400 MHz, CDCl3) δ 8.59 (s, 1 H), 8.42 (s, 1 H), 8.34 (d, J = 4.8 Hz, 1 H), 7.26 (d, J = 4.8 Hz, 1 H), 6.90 (t, J = 51.6 Hz, 1 H), 5.63-5.37 (m, 2 H), 3.13-3.06 (m, 1 H), 2.92-2.80 (m, 2 H), 1.36 (d, J = 6.4 Hz, 3 H) LCMS RT = 0.583 min, m/z = 378.2. | 0.036 |
| | 1-({5-[5-(difluoromethyl)-1,3,4- | | |
| | oxadiazol-2-yl]-1,3-thiazol-2- | | |
| | yl}methyl)-3-methyl-1,2,3,4- | | |
| | tetrahydro-1,7-naphthyridin-2-one | | |
| I-256 | 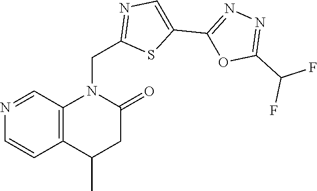 [SMILES: CC1CC(=O)N(Cc2ncc(-c3nnc(C(F)F)o3)s2)c2cnccc21] | 1H NMR (400 MHz, CDCl3) δ 8.58 (s, 1 H), 8.45-8.34 (m, 2 H), 7.25 (d, J = 4.8 Hz, 1 H), 6.89 (t, J = 51.6 Hz, 1 H), 5.62- 5.47 (m, 2 H), 3.27-3.20 (m, 1 H), 2.94-2.82 (m, 1 H), 2.67-2.56 (m, 1 H), 1.40 (d, J = 6.8 Hz, 3 H) LCMS RT = 0.580 min, m/z = 378.2. | 0.057 |
| | 1-({5-[5-(difluoromethyl)-1,3,4- | | |
| | oxadiazol-2-yl]-1,3-thiazol-2- | | |
| | yl}methyl)-4-methyl-1,2,3,4- | | |
| | tetrahydro-1,7-naphthyridin-2-one | | |
| I-257 | 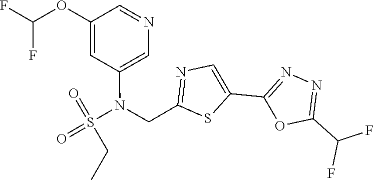 [SMILES: CCS(=O)(=O)N(Cc1ncc(-c2nnc(C(F)F)o2)s1)c1cncc(OC(F)F)c1] | 1H NMR (400 MHz, CD3OD δ 8.48 (d, J = 2.4 Hz, 1 H), 8.40 (d, J = 2.4 Hz, 1 H), 7.90 (s, 1 H), 7.77 (t, J = 2.4 Hz, 1 H), 7.23 (t, J = 51.6 Hz, 1 H), 6.97 (t, J = 72.8 Hz, 1 H), 5.35 (s, 2 H), 3.31 (q, J = 7.6 Hz, 2 H), 1.41 (t, J = 7.6 Hz, 3 H) LCMS RT = 1.251 min, m/z = 468.2. | 0.231 |
| | N-[5-(difluoromethoxy)pyridin-3- | | |
| | yl]-N-({2-[5-(difluoromethyl)- | | |
| | 1,3,4-oxadiazol-2-yl]-1,3-thiazol- | | |
| | 5-yl}methyl)ethane-1-sulfonamide | | |
| I-258 | 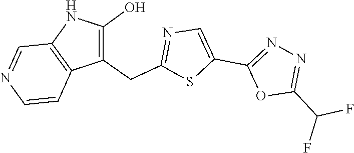 [SMILES: Oc1[nH]c2cnccc2c1Cc1ncc(-c2nnc(C(F)F)o2)s1] | 1H NMR (400 MHz, CD3OD) δ 8.40 (s, 1 H), 7.65 (s, 1 H), 7.50 (d, J = 6.8 Hz, 1 H), 7.17 (t, J = 51.6 Hz, 1 H), 6.94 (d, J = 6.8 Hz, 1 H), 4.27 (s, 2 H) LCMS RT = 0.778 min, m/z = 350.2. | 0.069 |
| | 3-({5-[5-(difluoromethyl)-1,3,4- | | |
| | oxadiazol-2-yl]-1,3-thiazol-2- | | |
| | yl}methyl)-1H-pyrrolo[2,3- | | |
| | c]pyridin-2-ol | | |
| I-259 | 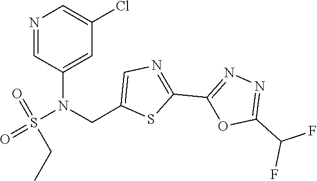 [SMILES: CCS(=O)(=O)N(Cc1cnc(-c2nnc(C(F)F)o2)s1)c1cncc(Cl)c1] | 1H NMR (400 MHz, CD3OD) δ 8.55-8.51 (m, 2 H), 8.05 (t, J = 2.4 Hz, 1 H), 7.92 (s, 1 H), 7.25 (t, J = 51.6 Hz, 1 H), 5.36 (s, 2 H), 3.34 (q, J = 7.2 Hz, 2 H), 1.42 (t, J = 7.6 Hz, 3 H) LCMS RT = 1.273 min, m/z = 436.2. | 0.121 |
| | N-(5-chloropyridin-3-yl)-N-({2- | | |
| | [5-(difluoromethyl)-1,3,4- | | |
| | oxadiazol-2-yl]-1,3-thiazol-5- | | |
| | yl}methyl)ethane-1-sulfonamide | | |
| I-260 | 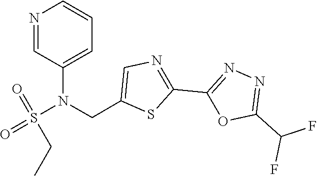 [SMILES: CCS(=O)(=O)N(Cc1cnc(-c2nnc(C(F)F)o2)s1)c1cccnc1] | 1H NMR (400 MHz, CDCl3) δ 8.58 (d, J = 2.4 Hz, 1 H), 8.52- 8.48 (m, 1 H), 7.98 (s, 1 H), 7.87-7.66 (m, 1 H), 7.56 (t, J = 51.2 Hz, 1 H), 7.45-7.42 (m, 1 H), 5.34 (s, 2 H), 3.37 (q, J = 7.2 Hz, 2 H), 1.30 (t, J = 7.2 Hz, 3 H) LCMS RT = 0.821 min, m/z = 402.2. | 0.333 |
| | N-({2-[5-(difluoromethyl)-1,3,4- | | |
| | oxadiazol-2-yl]-1,3-thiazol-5- | | |
| | yl}methyl)-N-(pyridin-3- | | |
| | yl)ethane-1-sulfonamide | | |
| I-261 | 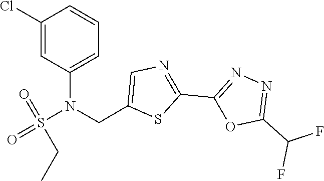 [SMILES: CCS(=O)(=O)N(Cc1cnc(-c2nnc(C(F)F)o2)s1)c1cccc(Cl)c1] | 1H NMR (400 MHz, CDCl3) δ 8.00 (s, 1 H), 7.56 (t, J = 51.2 Hz, 1 H), 7.41-7.36 (m, 4 H), 5.32 (s, 2 H), 3.36 (q, J = 7.2 Hz, 2 H), 1.29 (t, J = 8.0 Hz, 3 H) LCMS RT = 1.554 min, m/z = 435.1. | 0.089 |
| | N-(3-chlorophenyl)-N-({2-[5- | | |
| | (difluoromethyl)-1,3,4-oxadiazol- | | |
| | 2-yl]-1,3-thiazol-5- | | |
| | yl}methyl)ethane-1-sulfonamide | | |
| I-262 | 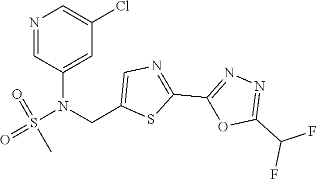 [SMILES: CS(=O)(=O)N(Cc1cnc(-c2nnc(C(F)F)o2)s1)c1cncc(Cl)c1] | 1H NMR (400 MHz, CD3OD) δ 8.61-8.47 (m, 2 H), 8.05 (t, J = 2.4 Hz, 1 H), 7.93 (s, 1 H), 7.23 (t, J = 51.6 Hz, 1 H), 5.32 (s, 2 H), 3.16 (s, 3 H) LCMS RT = 1.643 min, m/z = 422.1. | 0.110 |
| | N-(5-chloropyridin-3-yl)-N-({2- | | |
| | [5-(difluoromethyl)-1,3,4- | | |
| | oxadiazol-2-yl]-1,3-thiazol-5- | | |
| | yl}methyl)methanesulfonamide | | |
| I-263 | 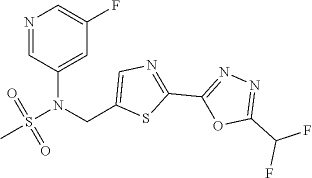 [SMILES: CS(=O)(=O)N(Cc1cnc(-c2nnc(C(F)F)o2)s1)c1cncc(F)c1] | 1H NMR (400 MHz, CD3OD) δ 8.48 (s, 1 H), 8.46 (d, J = 2.4 Hz, 1 H), 7.93 (s, 1 H), 7.88- 7.82 (m, 1 H), 7.23 (t, J = 51.6 Hz, 1 H), 5.33 (s, 2 H), 3.16 (s, 3 H) LCMS RT = 1.478 min, m/z = 406.1. | 0.108 |
| | N-({2-[5-(difluoromethyl)-1,3,4- | | |
| | oxadiazol-2-yl]-1,3-thiazol-5- | | |
| | yl}methyl)-N-(5-fluoropyridin-3- | | |
| | yl)methanesulfonamide | | |
| I-264 | 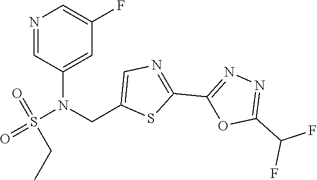 [SMILES: CCS(=O)(=O)N(Cc1cnc(-c2nnc(C(F)F)o2)s1)c1cncc(F)c1] | 1H NMR (400 MHz, CD3OD) δ 8.48 (s, 1 H), 8.45 (d, J = 2.8 Hz, 1 H), 7.91 (s, 1 H), 7.86- 7.80 (m, 1 H), 7.24 (t, J = 51.6 Hz, 1 H), 5.35 (s, 2 H), 3.31 (q, J = 7.2 Hz, 2 H), 1.41 (t, J = 7.6 Hz, 3 H). LCMS RT = 1.618 min, m/z = 420.2. | 0.087 |
| | N-({2-[5-(difluoromethyl)-1,3,4- | | |
| | oxadiazol-2-yl]-1,3-thiazol-5- | | |
| | yl}methyl)-N-(5-fluoropyridin-3- | | |
| | yl)ethane-1-sulfonamide | | |
| I-265 | 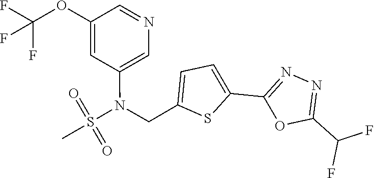 [SMILES: CS(=O)(=O)N(Cc1ccc(-c2nnc(C(F)F)o2)s1)c1cncc(OC(F)(F)F)c1] | 1H NMR (400 MHz, CDCl3) δ 8.72 (d, J = 2.0 Hz, 1 H), 8.56 (s, 1 H), 8.44 (s, 1 H), 7.80 (s, 1 H), 6.92 (t, J = 51.6 Hz, 1 H), 5.30 (s, 2 H), 3.11 (s, 3 H) LCMS RT = 1.950 min, m/z = 472.1. | 0.048 |
| | N-({5-[5-(difluoromethyl)-1,3,4- | | |
| | oxadiazol-2-yl]-1,3-thiazol-2- | | |
| | yl}methyl)-N-[5- | | |
| | (trifluoromethoxy)pyridin-3- | | |
| | yl]methanesulfonamide | | |
| I-266 | 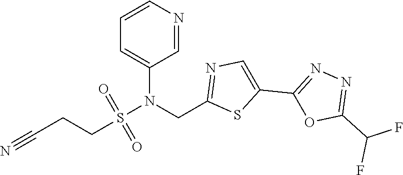 [SMILES: N#CCCS(=O)(=O)N(Cc1ncc(-c2nnc(C(F)F)o2)s1)c1cccnc1] | 1H NMR (400 MHz, CD3OD) δ 8.70 (d, J = 2.8 Hz, 1 H), 8.52 (d, J = 3.6 Hz, 1 H), 8.44 (s, 1 H), 8.07-8.01 (m, 1 H), 7.54-7.49 (m, 1 H), 7.21 (t, J = 51.6 Hz, 1 H), 5.41 (s, 2 H), 3.72 (t, J = 7.2 Hz, 2 H), 3.05 (t, J = 7.2 Hz, 2 H). LCMS RT = 1.287 min, m/z = 426.8. | 0.042 |
| | 2-cyano-N-({5-[5- | | |
| | (difluoromethyl)-1,3,4-oxadiazol- | | |
| | 2-yl]-1,3-thiazol-2-yl}methyl)-N- | | |
| | (pyridin-3-yl)ethane-1- | | |
| | sulfonamide | | |
| I-267 | 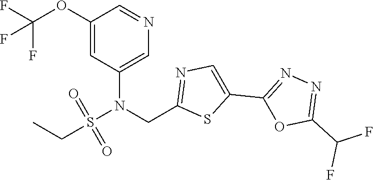 [SMILES: CCS(=O)(=O)N(Cc1ncc(-c2nnc(C(F)F)o2)s1)c1cncc(OC(F)(F)F)c1] | 1H NMR (400 MHz, CDCl3) δ 8.71 (s, 1 H), 8.54 (s, 1 H), 8.41 (s, 1 H), 7.79 (s, 1 H), 6.92 (t, J = 51.6 Hz, 1 H), 5.30 (s, 2 H), 3.22 (q, J = 7.6 Hz, 2 H), 1.45 (t, J = 7.6 Hz, 3 H) LCMS RT = 0.328 min, m/z = 486.1 | 0.044 |
| | N-({5-[5-(difluoromethyl)-1,3,4- | | |
| | oxadiazol-2-yl]-1,3-thiazol-2- | | |
| | yl}methyl)-N-[5- | | |
| | (trifluoromethoxy)pyridin-3- | | |
| | ylJethane-1-sulfonamide | | |
| I-268 | 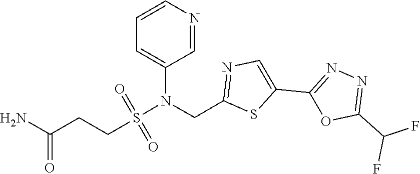 [SMILES: NC(=O)CCS(=O)(=O)N(Cc1ncc(-c2nnc(C(F)F)o2)s1)c1cccnc1] | 1H NMR (400 MHz, CDCl3) δ 8.75 (d, J = 2.4 Hz, 1 H), 8.62- 8.55 (m, 1 H), 8.39 (s, 1 H), 7.90-7.84 (m, 1 H), 7.40- 7.33 (m, 1 H), 6.91 (t, J = 52.0 Hz, 1 H), 5.27 (s, 2 H), 3.58 (t, J = 7.2 Hz, 2 H), 2.81 (t, J = 7.2 Hz, 2 H). LCMS RT = 0.783 min, m/z = 444.9. | 0.013 |
| | 3-[({5-[5-(difluoromethyl)-1,3,4- | | |
| | oxadiazol-2-yl]-1,3-thiazol-2- | | |
| | yl}methyl)(pyridin-3- | | |
| | yl)sulfamoyl]propanamide | | |
| I-269 | 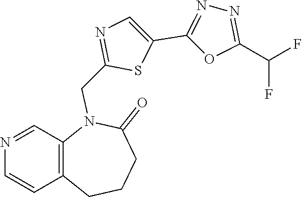 [SMILES: O=C1CCCc2ccncc2N1Cc1ncc(-c2nnc(C(F)F)o2)s1] | 1H NMR (400 MHz, CDCl3) δ 8.77 (s, 1 H), 8.46 (s, 1 H), 8.38 (s, 1 H), 7.28 (s, 1 H), 6.91 (t, J = 51.6 Hz, 1 H), 5.36 (s, 2 H), 2.84 (t, J = 6.8 Hz, 2 H), 2.49-2.37 (m, 2 H), 2.37- 2.23 (m, 2 H). LCMS RT = 1.043 min, m/z = 378.2 | 0.018 |
| | 1-({5-[5-(difluoromethyl)-1,3,4- | | |
| | oxadiazol-2-yl]-1,3-thiazol-2- | | |
| | yl}methyl)-1H,2H,3H,4H,5H- | | |
| | pyrido[3,4-b]azepin-2-one | | |
| I-270 | 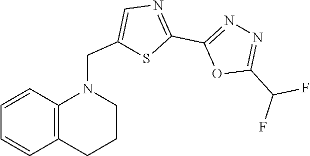 [SMILES: FC(F)c1nnc(-c2ncc(CN3CCCc4ccccc43)s2)o1] | 1H NMR (400 MHz, DMSO- d6) 8 8.57 (s, 1 H), 7.51 (t, J = 51.2 Hz, 1 H), 7.00-6.90 (m, 2 H), 6.60-6.55 (m, 2 H), 4.86 (s, 2 H), 3.47 (t, J = 5.6 Hz, 2 H), 2.75 (t, J = 6.0 Hz, 2 H), 2.02-1.92 (m, 2 H) LCMS RT = 1.769 min, m/z = 349.2 | 0.539 |
| | 1-({5-[5-(difluoromethyl)-1,3,4- | | |
| | oxadiazol-2-yl]-1,3-thiazol-2- | | |
| | yl}methyl)-1,2,3,4- | | |
| | tetrahydroquinoline | | |

### Example 2. Synthesis, Biochemical Activity and Potency of Various HDAC6 Inhibitors of Formula (II)

The compounds disclosed herein, in particular those of Formula (II), were synthesized according to methods disclosed in WO 2021/067859, which is incorporated herein by reference in its entirety. These compounds were tested for potency against HDAC6 and selectivity against HDAC1 in a biochemical assay. A biochemical assay was adopted using a luminescent HDAC-Glo I/II assay (Promega) and measured the relative activity of HDAC6 and HDAC1 recombinant proteins. Compounds were first incubated in the presence of HDAC6 or HDAC1 separately, followed by addition of the luminescent substrate. The data was acquired using a plate reader and the biochemical IC₅₀ were calculated from the data accordingly. Data is tabulated in Table 4. From these studies, it was determined that the compounds of the present disclosure are selective inhibitors of HDAC6 over HDAC1, providing selectivity ratios from about 5 to about 30,0000.

TABLE 4
HDAC6 Activity for Disclosed Hydroxamic Acid
Compounds of Formula (II) and Formula (III).

| Compound ID | HDAC6 IC50 (nM) |
|---|---|
| 1 | 136 |
| II-1 | 12.5 |
| II-6 | 1.61 |
| II-4 | 16.7 |
| II-24 | 146 |
| II-5 | 6.68 |
| II-7A | 1.5 |
| II-7B | 4.13 |
| II-2 | 104 |
| II-10 | 76.6 |
| II-3 | 73 |
| III-1 | 8.26 |
| III-4 | 1.8 |
| II-9A | 0.351 |
| III-2 | 0.677 |
| III-3 | 3.35 |
| II-9B | 0.791 |
| II-11 | 0.639 |
| II-19 | 0.425 |
| II-18 | 1.68 |
| II-16 | 1.61 |
| II-8B | 0.275 |
| II-13 | 73 |
| II-14 | 28.3 |
| II-17 | 1.12 |
| 4 | 1.2 |
| II-25 | 0.669 |
| III-9 | 0.595 |
| II-21 | 0.601 |
| II-22 | 3.36 |
| II-23 | 1.59 |
| I-1a | 1.79 |
| III-5 | 2.04 |
| II-12B | 0.809 |
| III-10 | 2.3 |
| III-7 | 1.1 |
| III-6 | 4.06 |
| III-8 | 10.3 |
| II-15 | 2.64 |
| II-20 | 3.78 |
| II-8A | 1.65 |
| 5 | 1.49 |
| II-26A | 4.57 |
| II-26B | 2.23 |
| 6 | 2.54 |
| II-27 | 1.13 |
| II-28A | 1.81 |
| II-29 | 13.2 |
| II-30A | 1.36 |
| II-30B | 7.91 |
| II-31 | 13.8 |
| II-32 | 13 |
| II-33 | 27.9 |
| II-34 | 2.32 |
| II-35 | 21.7 |
| II-36 | 5.22 |

The structures, chemical names and characterization of the compounds of Formula (11) described in Example 2 are provided in Table 5.

TABLE 5
Structure, chemical names and characterization for Compounds of Formula
(II) and Formula (III).

| Amine | Compound | Characterization Data |
|---|---|---|
| 2-amino- N, 2- dimethyl propanamide | 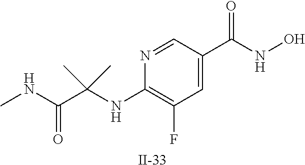 [SMILES: CNC(=O)C(C)(C)Nc1ncc(C(=O)NO)cc1F ; "II-33"] | ¹H NMR (400 MHz, MeOH-d4) δ 8.24 (s, 1 H), 7.62 (s, 1 H), 7.59 (s, 1 H), 2.69-2.65 (m, 3 H), 1.59 (s, 6 H). LC-MS: t_{R} (min) 1.32 (20-100% ACN with 0.1% TFA 6 min), m/z [M + H]⁺ C₁₁H₁₅FN₄O₂ requires: 270.3, found: 271.1 HPLC t_{R} (min) 1.57, 95% (20-100% ACN with 0.1% TFA 10 min.) |
| 3-amino- 1- methylpiperidin- 2-one | 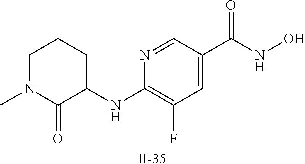 [SMILES: CN1CCCC(Nc2ncc(C(=O)NO)cc2F)C1=O ; "II-35"] | ¹H NMR (400 MHz, DMSO-d6) δ 11.1-10.9 (br, s, 1 H), 8.98 (s, 1 H), 8.25 (s, 1 H), 7.73-7.53 (m, 1 H), 7.29-7.04 (m, 1 H), 4.80-4.50 (m, 1 H), 3.33-3.28 (m, 2 H), 2.83 (s, 3 H), 2.16-2.00 (m, 1 H), 1.90 (br, s, 3 H). LC-MS: tR (min) 1.28 (20-100% ACN with 0.1% TFA 6 min), m/z [M + H]⁺ C₁₂H₁₅FN₄O₂ requires: 282.3, found: 283.1 HPLC tR (min) 3.66, 95% (20-100% ACN with 0.1% TFA 10 min.) |
| (S)-1- cyclopropylethan- 1-amine | 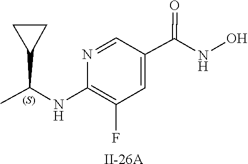 | ¹H NMR (400 MHz, MeOH-d4) δ 7.98 (s, 1 H), 7.32 (d, J = 1.71 Hz, 1 H), 7.29 (d, J = 1.71 Hz, 1 H), 3.45- 3.35 (m, 1 H), 1.07 (d, J = 6.60 Hz, 3 H), 0.86-0.74 (m, 1 H), 0.33-0.01 (m, 4 H). LC-MS: t_{R} (min) 2.03 (20-100% ACN with 0.1% TFA 6 min), m/z [M + H]⁺ C₁₁H₁₂FN₃O₂ requires: 239.3, found: 240.1 HPLC t_{R} (min) 4.37, 95% (20-100% ACN with 0.1% TFA 10 min.) |
| (R)-1- cyclopropylethan- 1-amine | 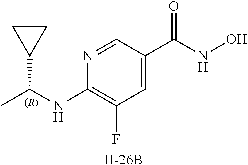 | ¹H NMR (400 MHz, MeOH-d4) δ 7.98 (s, 1 H), 7.32 (d, J = 1.71 Hz, 1 H), 7.29 (d, J = 1.47 Hz, 1 H), 3.10- 3.07 (m, 1 H), 1.07 (d, J = 6.60 Hz, 3 H), 0.90-0.70 (m, 1 H), 0.33-0.00 (m, 4 H). LC-MS: t_{R} (min) 2.03 (20-100% ACN with 0.1% TFA 6 min), m/z [M + H]⁺ C₁₁H₁₂FN₃O₂ requires: 239.3, found: 240.1 HPLC t_{R} (min) 3.74, 95% (20-100% ACN with 0.1% TFA 10 min.) |
| (R)-1- phenylpropan-1- amine | 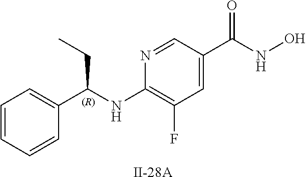 | ¹H NMR (400 MHz, MeOH-d4) δ 8.20 (s, 1 H), 7.56 (d, J = 11.7 Hz, 1 H), 7.39 (d, J = 7.58 Hz, 2 H), 7.29 (t, J = 7.46 Hz, 2 H), 7.24-7.11 (m, 1 H), 5.15-5.02 (m, 1 H), 2.00-1.75 (m, 2 H), 1.02-0.90 (m, 3 H). LC-MS: t_{R} (min) 3.70 (20-100% ACN with 0.1% TFA 6 min), m/z [M + H]⁺ C₁₃H₁₅FN₃O₂ requires: 289.3, found: 290.1 HPLC t_{R} (min) 5.23, 98% (20-100% ACN with 0.1% TFA 10 min.) |
| (R)-1- cyclohexyl- ethylamine | 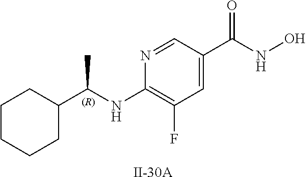 | ¹H NMR (400 MHz, MeOH-d4) δ 8.24 (s, 1H), 7.56 (s, 1 H), 7.53 (s, 1 H), 4.63 (s, 2 H), 4.09 (t, J = 6.72 Hz, 1 H), 1.88-1.65 (m, 5 H), 1.55-1.45 (m, 1 H), 1.31-1.22 (m, 3 H), 1.22-1.15 (m, 3 H), 1.10-0.90 (m, 2 H). LC-MS: t_{R} (min) 3.37 (20-100% ACN with 0.1% TFA 6 min), m/z [M + H]⁺ C₁₄H₂₀FN₃O₂ requires: 281.3, found: 282.2 HPLC t_{R} (min) 4.84, 97% (20-100% ACN with 0.1% TFA 10 min.) |
| (S)-1- cyclohexyl- ethylamine | 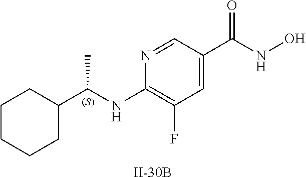 | ¹H NMR (400 MHz, MeOH-d4) δ 8.24 (s, 1H), 7.56 (s, 1 H), 7.53 (s, 1 H), 4.63 (s, 2 H), 4.09 (t, J = 6.97 Hz, 1 H), 1.88-1.65 (m, 5 H), 1.51 (s, 1 H), 1.31-1.20 (m, 3 H), 1.22-1.18 (m, 3 H), 1.10-0.90 (m, 2 H). LC-MS: t_{R} (min) 3.50 (20-100% ACN with 0.1% TFA 6 min), m/z [M + H]⁺ C₁₄H₂₀FN₃O₂ requires: 281.3, found: 282.1 HPLC t_{R} (min) 5.10, 98% (20-100% ACN with 0.1% TFA 10 min.) |
| (1S, 2S)- 2- methoxycyclo- pentylamine | 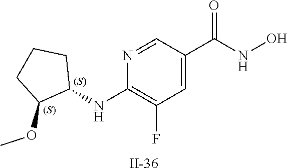 | ¹H NMR (400 MHz, CDCl3-d) δ 8.27 (s, 1 H), 7.58- 7.39 (m, 1 H), 5.06 (br, s, 1 H), 4.31 (br, s, 1 H), 3.68 (br, s, 1 H), 3.34 (s, 3 H), 2.25- 2.15 (m, 1 H), 1.93- 1.88 (m, 1 H), 1.85-1.75 (m, 3 H), 1.50-1.40 (m, 1 H). LC-MS: t_{R} (min) 1.93 (20-100% ACN with 0.1 % TFA 6 min), m/z [M + H]⁺ C₁₂H₁₆FN₃O₂ requires: 269.3, found: 270.1 HPLC t_{R} (min) 3.71, 97% (20-100% ACN with 0.1% TFA 10 min.) |
| (3S, 4R)- 4- methoxytetrahydro furan-3- yl)amine | 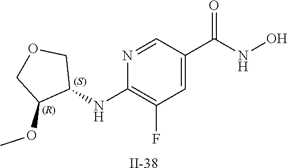 | ¹H NMR (400 MHz, MeOH-d4) δ 8.33 (s, 1 H), 7.60 (d, J = 11.98 Hz, 1 H), 4.60 (br, s, 1 H), 4.20-4.05 (m, 2 H), 4.00-3.90 (m, 1 H), 3.85-3.75 (m, 2 H), 3.48 (s, 3 H). LC-MS: t_{R} (min) 1.39 (20-100% ACN with 0.1% TFA 6 min), m/z [M + H]⁺ C₁₁H₁₄FN₃O₂ requires: 271.3, found: 272.1 HPLC t_{R} (min) 2.97, 97% (20-100% ACN with 0.1% TFA 10 min.) |
| 3,3- difluoro- 1- (methoxymethyl) cyclobutan- 1-amine | 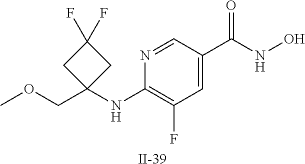 [SMILES: COCC1(Nc2ncc(C(=O)NO)cc2F)CC(F)(F)C1 ; "II-39"] | ¹H NMR (400 MHz, MeOH-d4) δ 8.16 (s, 1 H), 7.48 (d, J = 12.2 Hz, 1 H), 4.52 (s, 2 H), 3.63 (s, 2 H), 3.26- 3.23 (m, 3 H), 2.82-2.72 (m, 4H). LC-MS: t_{R} (min) 3.00 (20-100% ACN with 0.1% TFA 6 min), m/z [M + H]⁺ C₁₂H₁₄F₃N₃O₃ requires: 305.3, found: 306.1 HPLC t_{R} (min) 5.02, 93% (20-100% ACN with 0.1% TFA 10 min.) |
| 3- ethyloxetan-3- amine | 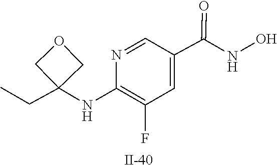 | ¹H NMR (400 MHz, MeOH-d4) δ 8.22 (s, 1 H), 7.59 (d, J = 11.98 Hz, 1 H), 4.80 (br, s, 2 H), 4.60 (br, s, 2 H), 2.22 (q, J = 6.93 Hz, 2 H), 0.92 (t, J = 6.95 Hz, 3 H). LC-MS: t_{R} (min) 1.10 (20-100% ACN with 0.1% TFA 6 min), m/z [M + H]⁺ C₁₁H₁₃FN₃O₃ requires: 255.2, found: 256.1 HPLC t_{R} (min) 3.80, 99% (20-100% ACN with 0.1% TFA 10 min.) |
| 1,1′- bi(cyclopropan)- 1- amine | 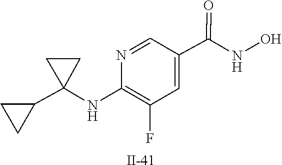 | ¹H NMR (400 MHz, MeOH-d4) δ 8.07 (s, 1 H), 7.32 (d, J = 11.98 Hz, 1 H), 4.40 (br, s, 1 H), 3.08 (s, 1 H), 1.35-1.25 (m, 1 H), 0.48 (d, J = 5.38 Hz, 4 H), 0.38- 0.42 (m, 2 H), 0.01 (s, 2 H). LC-MS: t_{R} (min) 1.20 (20-100% ACN with 0.1% TFA 6 min), m/z [M + H]⁺ C₁₁H₁₃FN₃O₃ requires: 251.3, found: 252.1 HPLC t_{R} (min) 3.17, 96% (20-100% ACN with 0.1% TFA 10 min.) |
| (S)-1- methyl-2- oxopyrrolidin-3- amine | 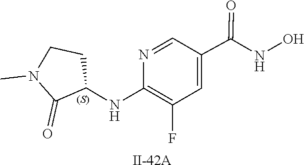 | ¹H NMR (400 MHz, DMSO-d6) δ 11.05 (br, s, 1 H), 9.00 (br, s, 1 H), 8.50 (br, s, 1 H), 7.65 (d, J = 12.0 Hz, 1 H), 7.45 (d, J = 8.80 Hz, 1 H), 4.90-4.80 (m, 1 H), 3.45-3.40 (m, 2 H), 2.76 (s, 3 H), 2.30-2.20 (m, 1 H), 1.90-2.00 (m, 1 H). LC-MS: t_{R} (min) 1.10 (20-100% ACN with 0.1% TFA 6 min), m/z [M + H]⁺ C₁₁H₁₃FN₃O₃ requires: 268.3, found: 269.1 HPLC t_{R} (min) 1.54, 96% (20-100% ACN with 0.1% TFA 10 min.) |
| (S)-2- oxopyrrolidin-3- amine | 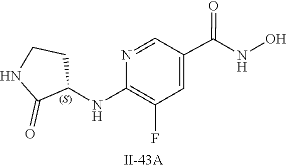 | ¹H NMR (400 MHz, DMSO-d6) δ 11.05 (br, s, 1 H), 8.95 (br, s, 1 H), 8.25 (s, 1 H), 7.83 (s, 1 H), 7.65 (d, J = 12.0 Hz, 1 H), 7.45 (d, J = 8.80 Hz, 1 H), 4.70-4.85 (m, 1 H), 3.23 (d, J = 6.34 Hz, 2 H), 2.40-2.30 (m, 1 H), 2.15-2.05 (m, 1 H). LC-MS: t_{R} (min) 1.10 (20-100% ACN with 0.1% TFA 6 min), m/z [M + H]⁺ C₁₂H₁₆FN₃O₃ requires: 254.2, found: 255.1 HPLC t_{R} (min) 1.71, 96% (20-100% ACN with 0.1% TFA 10 min.) |
| (R)-1- methyl-2- oxopyrrolidin-3- amine | 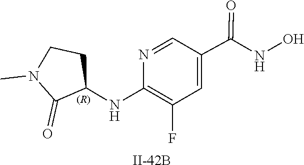 | ¹H NMR (400 MHz, DMSO-d6) δ 11.05 (br, s, 1 H), 8.99 (br, s, 1 H), 8.25 (s, 1 H), 7.65 (d, J = 12.2 Hz, 1 H), 7.42 (d, d, J = 8.07 Hz, 1 H), 4.82 (d, J = 9.05 Hz, 1 H), 3.34-3.27 (m, 2 H), 2.76 (s, 3 H), 2.40-2.26 (m, 1 H), 2.03-1.92 (m, 1 H). LC-MS: t_{R} (min) 1.20 (20-100% ACN with 0.1% TFA 6 min), m/z [M + H]⁺ C₁₂H₁₆FN₃O₃ requires: 268.3, found: 269.1 HPLC t_{R} (min) 1.75, 99% (20-100% ACN with 0.1% TFA 10 min.) |
| (R)-2- oxopyrrolidin-3- amine | 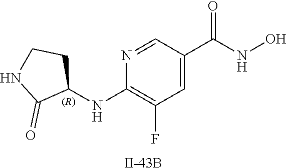 | ¹H NMR (400 MHz, DMSO-d6) δ 11.0 (br, s, 1 H), 9.05 (br, s, 1 H), 8.45 (s, 1 H), 7.84 (s, 1 H), 7.65 (d, J = 12.0 Hz, 1 H), 7.34 (d, J = 8.80 Hz, 1 H), 4.77 (d, J = 9.29 Hz, 1 H), 3.18-3.28 (m, 2 H), 2.30-2.40 (m, 1 H), 2.15-2.05 (m, 1 H). LC-MS: t_{R} (min) 1.15 (20-100% ACN with 0.1% TFA 6 min), m/z [M + H]⁺ C₁₂H₁₆FN₃O₃ requires: 254.2, found: 255.1 HPLC t_{R} (min) 1.55, 93% (20-100% ACN with 0.1% TFA 10 min.) |
| 1-(2- methoxyethyl) cyclopropyl) amine | 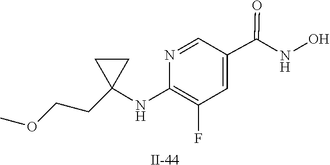 [SMILES: COCCC1(Nc2ncc(C(=O)NO)cc2F)CC1 ; "II-44"] | ¹H NMR (400 MHz, MeOH-d4) δ 8.32 (s, 1 H), 7.57 (d, J = 11.98 Hz, 1 H), 3.55 (t, J = 6.85, 2 H), 3.30 (s, 3 H), 1.96 (t, J = 6.85 Hz, 2 H), 0.80 (s, 4 H). LC-MS: t_{R} (min) 1.20 (20-100% ACN with 0.1% TFA 6 min), m/z [M + H]⁺ C₁₂H₁₆FN₃O₃ requires: 269.3, found: 270.1 HPLC t_{R} (min) 2.07, 99% (20-100% ACN with 0.1% TFA 10 min.) |
| 3- phenyloxetan-3- amine | 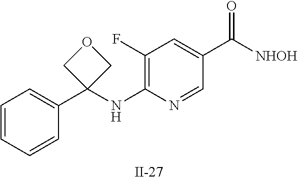 | ¹H NMR (400 MHz, DMSO-d6) δ 10.95 (br s, 1 H), 8.97 (s, 1 H), 8.38 (m, 1 H), 8.02 (m, 1 H), 7.69 (m, 1 H), 7.53 (m, 2 H), 7.34 (m, 2 H), 7.24 (m, 1 H), 4.99 (m, 2 H), 4.78 (m, 2 H). LC-MS: m/z [M + H]⁺ C₁₅H₁₄FN₃O₃ requires: 303.2, found: 304.1 HPLC t_{R} (min) 4.81, 95% (10-100% ACN with 0.1% TFA 10 min.) |
| 3- (pyridin- 2- yl)oxetan- 3-amine | 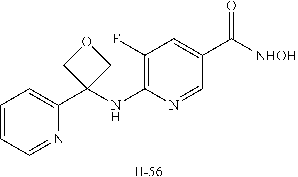 | ¹H NMR (400 MHz, DMSO-d6) δ 10.97 (br s, 1 H), 8.97 (s, 1 H), 8.63 (m, 1 H), 8.46 (m, 1 H), 7.99 (m, 1 H), 7.71-7.66 (m, 2 H), 7.31-7.15 (m, 2 H), 5.01 (m, 2 H), 4.87 (m, 2 H). LC-MS: m/z [M + H]⁺ C₁₄H₁₃FN₄O₃ requires: 304.2, found: 305.1 HPLC tR (min) 1.27, 93% (10-100% ACN with 0.1% TFA 10 min.) |
| 1H- pyrrolo [2,3- b]pyridine | 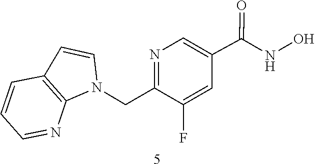 | ¹H NMR (400 MHz, DMSO-d6) δ 11.41 (br s, 1 H), 9.31 (s, 1 H), 8.57 (m, 1 H), 8.18 (m, 1 H), 7.98 (m, 2 H), 7.60 (m, 1 H), 7.08 (m, 1 H), 6.52 (m, 1 H), 5.71 (s, 2 H). LC-MS: m/z [M + H]⁺ C₁₄H₁₁FN₄O₂ requires: 286.2, found: 287.1 HPLC t_{R} (min) 3.21, 98% (20-100% ACN with 0.1% TFA 10 min.) |
| 2- (difluoro methyl)- 1H- benzo[d] imidazole | 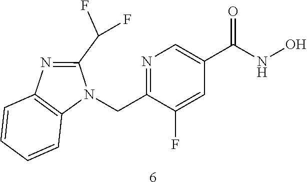 | ¹H NMR (400 MHz, DMSO-d6) δ 11.41 (br s, 1 H), 9.32 (s, 1 H), 8.51 (s, 1 H), 8.01 (m, 1 H), 7.63 (m, 1 H), 7.62 (m, 1 H), 7.53-7.27 (m, 3 H), 5.92 (s, 2 H). LC-MS: m/z [M + H]⁺ C₁₅H₁₁F₃N₄O₂ requires: 336.2.1, found: 337.1 HPLC t_{R} (min) 4.87, 97% (10-100% ACN with 0.1% TFA 10 min.) |
| 2,2- dimethyl- 2H- pyrido [3,2- b][1,4]oxazin- 3(4H)- one | 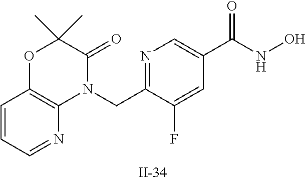 [SMILES: CC1(C)Oc2cccnc2N(Cc2ncc(C(=O)NO)cc2F)C1=O ; "II-34"] | ¹H NMR (400 MHz, DMSO-d6) δ 11.37 (br s, 1 H), 9.30 (s, 1 H), 8.52 (s, 1 H), 7.97 (m, 1 H), 7.88 (m, 1 H), 7.44 (m, 1 H), 7.03 (m, 1 H), 5.42 (s, 2 H), 1.51 (s, 6 H). LC-MS: m/z [M + H]⁺ C₁₆H₁₅FN₄O₄ requires: 346.3, found: 347.1 HPLC t_{R} (min) 5.04, 100% (10-100% ACN with 0.1% TFA 10 min.) |
| 3-methyl- 3,4- dihydroquinazolin- 2(1H)- one | 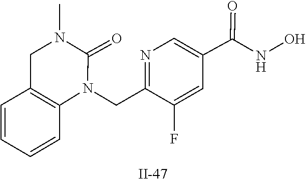 [SMILES: CN1Cc2ccccc2N(Cc2ncc(C(=O)NO)cc2F)C1=O ; "II-47"] | ¹H NMR (400 MHz, DMSO-d6) δ 11.40 (br s, 1 H), 9.30 (s, 1 H), 8.55 (s, 1 H), 7.94 (m, 1 H), 7.15-7.11 (m, 2 H), 6.93 (m, 1 H), 6.74 (m, 1 H), 5.22 (s, 2 H), 4.40 (s, 2 H), 2.90 (s, 3 H). LC-MS: m/z [M + H]⁺ C₁₆H₁₅FN₄O₃ requires: 330.3, found: 331.1 HPLC t_{R} (min) 4.93, 100% (10-100% ACN with 0.1% TFA 10 min.) |
| 3,4- dihydro- 2H- thieno [3,2- b]indole 1,1- dioxide | 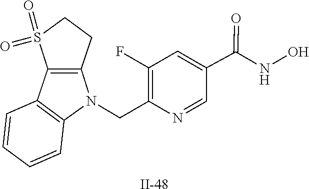 [SMILES: O=C(NO)c1cnc(Cn2c3c(c4ccccc42)S(=O)(=O)CC3)c(F)c1 ; "II-48"] | ¹H NMR (400 MHz, DMSO-d6) δ 8.62 (s, 1 H), 8.02 (m, 1 H), 7.56 (m, 2 H), 7.27 (m, 2 H), 5.68 (s, 2 H), 3.94 (m, 2 H), 3.46 (m, 2 H). LC-MS: m/z [M + H]⁺ C₁₇H₁₄FN₃O₄S requires: 375.3, found: 376.1 HPLC t_{R} (min) 4.85, 96% (10-100% ACN with 0.1% TFA 10 min.) |
| 2- (trifluoro methyl)- 1H- benzo[d] imidazole | 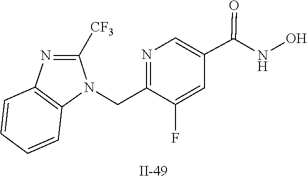 [SMILES: O=C(NO)c1cnc(Cn2c(C(F)(F)F)nc3ccccc32)c(F)c1 ; "II-49"] | ¹H NMR (400 MHz, DMSO-d6) δ 11.41 (br s, 1 H), 9.33 (s, 1 H), 8.49 (s, 1 H), 8.04 (m, 1 H), 7.84 (m, 1 H), 7.72 (m, 1 H), 7.44 (m, 2 H), 5.95 (s, 2 H). LC-MS: m/z [M + H]⁺ C₁₅H₁₀F₄N₄O₂ requires: 354.2, found: 355.1 HPLC t_{R} (min) 4.94, 98% (10-100% ACN with 0.1% TFA 10 min.) |
| 2-methyl- 1H- pyrrolo [2,3- b]pyridine | 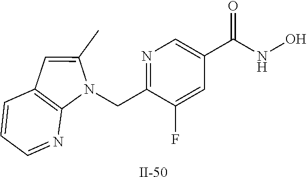 [SMILES: Cc1cc2cccnc2n1Cc1ncc(C(=O)NO)cc1F ; "II-50"] | ¹H NMR (400 MHz, DMSO-d6) δ 11.39 (br s, 1 H), 9.31 (s, 1 H), 8.51 (s, 1 H), 8.06 (m, 1 H), 7.97 (m, 1 H), 7.84 (m, 1 H), 7.03 (m, 1 H), 6.29 (s, 1 H), 5.69 (s, 2 H), 2.40 (s, 3 H). LC-MS: m/z [M + H]⁺ C₁₅H₁₃FN₄O₂ requires: 300.2, found: 301.1 HPLC t_{R} (min) 3.83, 100% (20-100% ACN with 0.1% TFA 10 min.) |
| 2,3- dimethyl- 1H- pyrrolo [2,3- b]pyridine | 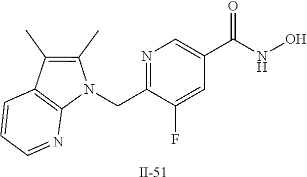 [SMILES: Cc1c(C)n(Cc2ncc(C(=O)NO)cc2F)c2ncccc12 ; "II-51"] | ¹H NMR (400 MHz, DMSO-d6) δ 11.39 (br s, 1 H), 9.31 (s, 1 H), 8.51 (s, 1 H), 8.05 (m, 1 H), 7.95 (m, 1 H), 7.81 (m, 1 H), 7.01 (m, 1 H), 5.67 (s, 2 H), 2.31 (s, 3 H), 2.21 (s, 3 H). LC-MS: m/z [M + H]⁺ C₁₆H₁₅FN₄O₂ requires: 314.3, found: 315.1 HPLC t_{R} (min) 4.17, 100% (20-100% ACN with 0.1% TFA 10 min.) |
| oxazolo [4,5- b]pyridin- 2(3H)- one | 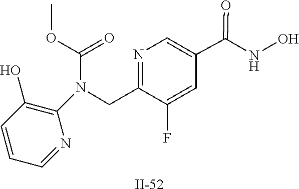 [SMILES: COC(=O)N(Cc1ncc(C(=O)NO)cc1F)c1ncccc1O ; "II-52"] | ¹H NMR (400 MHz, CD₃OD-d₄) δ 8.70 (s, 1 H), 7.98 (m, 2 H), 7.47 (m, 1 H), 7.38 (m, 1 H), 5.06 (s, 2 H), 3.64 (s, 3 H). LC-MS: m/z [M + H]⁺ C₁₄H₁₃FN₄O₅ requires: 336.2, found: 337.1 HPLC t_{R} (min) 4.18, 96% (10-100% ACN with 0.1% TFA 10 min.) |
| 2-methyl- 3H- imidazo [4,5- b]pyridine | 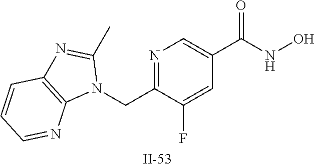 [SMILES: Cc1nc2cccnc2n1Cc1ncc(C(=O)NO)cc1F ; "II-53"] | ¹H NMR (400 MHz, DMSO-d6) δ 11.30 (br s, 1 H), 9.27 (s, 1 H), 8.47 (s, 1 H), 8.12 (m, 1 H), 7.95 (m, 1 H), 7.88 (m, 1 H), 7.15 (m, 1 H), 5.68 (s, 2 H), 2.44 (s, 3 H). LC-MS: m/z [M + H]⁺ C₁₄H₁₂FN₅O₂ requires: 301.2, found: 302.1 HPLC t_{R} (min) 3.60, 98% (10-100% ACN with 0.1% TFA 10 min.) |
| 4-(1H- benzo[d] imidazol- 2- yl)morpholine | 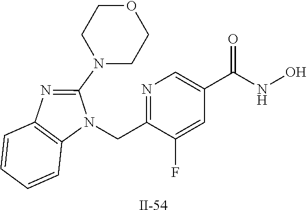 [SMILES: O=C(NO)c1cnc(Cn2c(N3CCOCC3)nc3ccccc32)c(F)c1 ; "II-54"] | ¹H NMR (400 MHz, DMSO-d6) δ 11.45 (br s, 1 H), 9.34 (s, 1 H), 8.59 (s, 1 H), 8.03 (m, 1 H), 7.45 (m, 1 H), 7.15-7.03 (m, 3 H), 5.55 (s, 2 H), 3.67 (m, 4 H), 3.14 (m, 4 H). LC-MS: m/z [M + H]⁺ C₁₈H₁₈FN₅O₃ requires: 371.3, found: 372.1 HPLC t_{R} (min) 3.66, 95% (10-100% ACN with 0.1% TFA 10 min.) |
| 2-methyl- 2,3,4,5- tetrahydro- 1H- pyrido [4,3- b]indole | 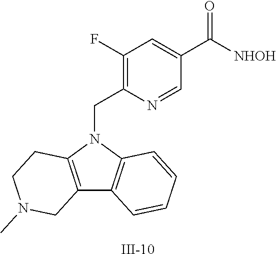 | ¹H NMR (400 MHz, DMSO-d6) δ 11.40 (br s, 1 H), 9.36 (s, 1 H), 8.61 (s, 1 H), 7.95 (m, 1 H), 7.35 (m, 2 H), 7.03-6.94 (m, 2 H), 5.50 (s, 2 H), 3.52 (s, 2 H), 2.83 (m, 2 H), 2.72 (m, 2 H), 2.42 (s, 3 H). LC-MS: m/z [M + H]⁺ C₁₉H₁₉FN₄O₂ requires: 354.3, found: 355.2 HPLC t_{R} (min) 4.58, 99% (10-100% ACN with 0.1% TFA 10 min.) |
| 1,3,4,5- tetrahydropyrano [4,3- b]indole | 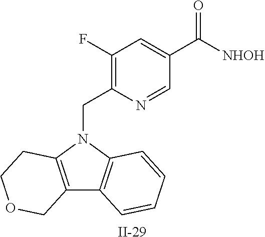 | ¹H NMR (400 MHz, DMSO-d6) δ 11.41 (br s, 1 H), 9.35 (s, 1 H), 8.61 (s, 1 H), 8.01 (m, 1 H), 7.40-7.32 (m, 2 H), 7.06-6.95 (m, 2 H), 5.54 (s, 2 H), 4.78 (s, 2 H), 3.95 (m, 2 H), 2.84 (m, 2 H). LC-MS: m/z [M + H]⁺ C₁₈H₁₆FN₃O₃ requires: 341.3, found: 342.1 HPLC t_{R} (min) 4.99, 94% (10-100% ACN with 0.1% TFA 10 min.) |

| Compound | Characterization Data |
|---|---|
| 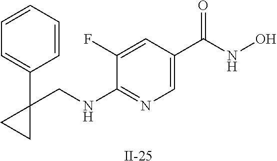 [SMILES: O=C(NO)c1cnc(NCC2(c3ccccc3)CC2)c(F)c1 ; "II-25"] | ¹H NMR (400 MHz, Solvent) δ ppm 8.18 (s, 1 H) 7.50 (br d, J = 11.98 Hz, 1 H) 7.31-7.38 (m, 2 H) 7.23 (br t, J = 7.46 Hz, 2 H) 7.10-7.17 (m, 1 H) 3.74 (s, 2 H) 0.93-0.99 (m, 2 H) 0.80-0.86 (m, 2 H) |
| 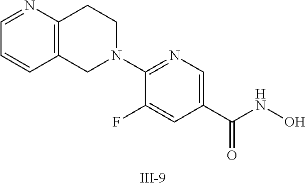 [SMILES: O=C(NO)c1cnc(N2CCc3ncccc3C2)c(F)c1 ; "III-9"] | ¹H NMR (400 MHz, Solvent) δ ppm 8.39 (s, 1 H) 8.35 (d, J = 4.65 Hz, 1 H) 7.64-7.76 (m, 2 H) 7.27 (dd, J = 7.58, 5.14 Hz, 1 H) 4.86 (s, 2 H) 4.03 (t, J = 5.87 Hz, 2 H) 3.10 (t, J = 5.87 Hz, 2 H) |
| 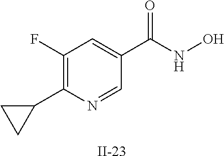 | ¹H NMR (400 MHz, METHANOL-d4) δ ppm 8.57 (s, 1 H) 7.77 (d, J = 10.27 Hz, 1 H) 2.31-2.46 (m, 1 H) 1.08-1.16 (m, 4 H) LCMS R_{T} = 2.79 min, m/z = 197.1 [M + H]⁺. |
| 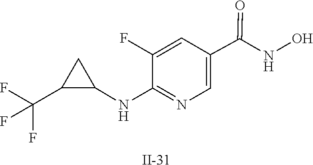 [SMILES: O=C(NO)c1cnc(NC2CC2C(F)(F)F)c(F)c1 ; "II-31"] | ¹H NMR (400 MHz, METHANOL-d4) δ ppm 8.34 (s, 1 H) 7.60 (br d, J = 11.49 Hz, 1 H) 3.22-3.28 (m, 1 H) 1.82-1.95 (m, 1 H) 1.21-1.31 (m, 1 H) 1.16 (br dd, J = 10.03, 4.89 Hz, 1 H) |
| 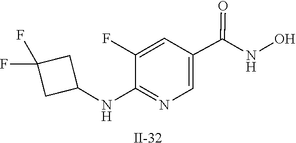 | ¹H NMR (400 MHz, METHANOL-d4) δ ppm 8.30 (s, 1 H) 7.61 (br d, J = 11.74 Hz, 1 H) 4.32-4.47 (m, 1 H) 2.94-3.12 (m, 2 H) 2.54-2.76 (m, 2 H) |
| 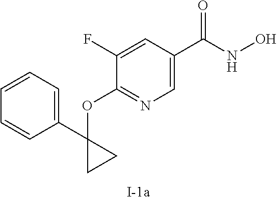 [SMILES: O=C(NO)c1cnc(OC2(c3ccccc3)CC2)c(F)c1] | ¹H NMR (400 MHz, METHANOL-d4) δ ppm 8.21 (s, 1 H) 7.82 (br d, J = 10.27 Hz, 1 H) 7.25-7.32 (m, 4 H) 7.15-7.23 (m, 1 H) 1.44-1.50 (m, 2 H) 1.36- 1.43 (m, 2 H) |

### Example 3. Materials & Methods

The HEK293/IEK293T cells used (the specific cell line used is indicated in the tables below) were maintained in serum-containing or serum-free cell culture medium (the specific conditions used are indicated in the tables below).

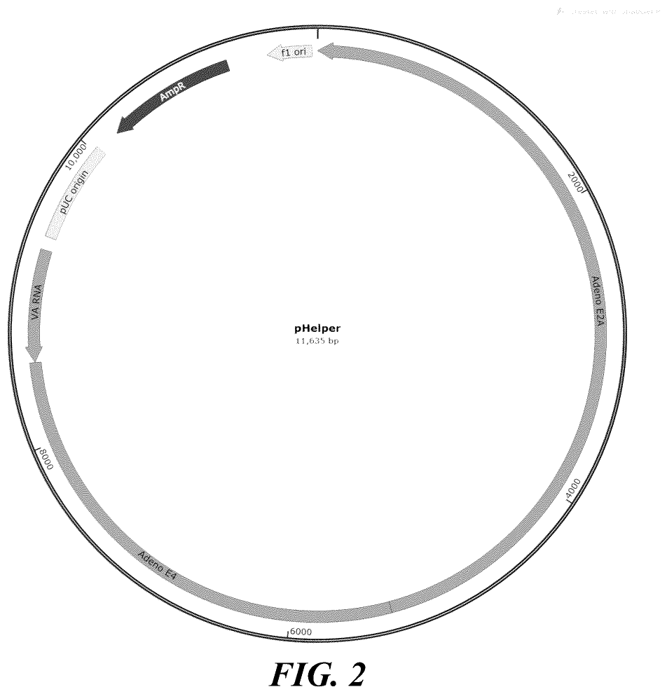

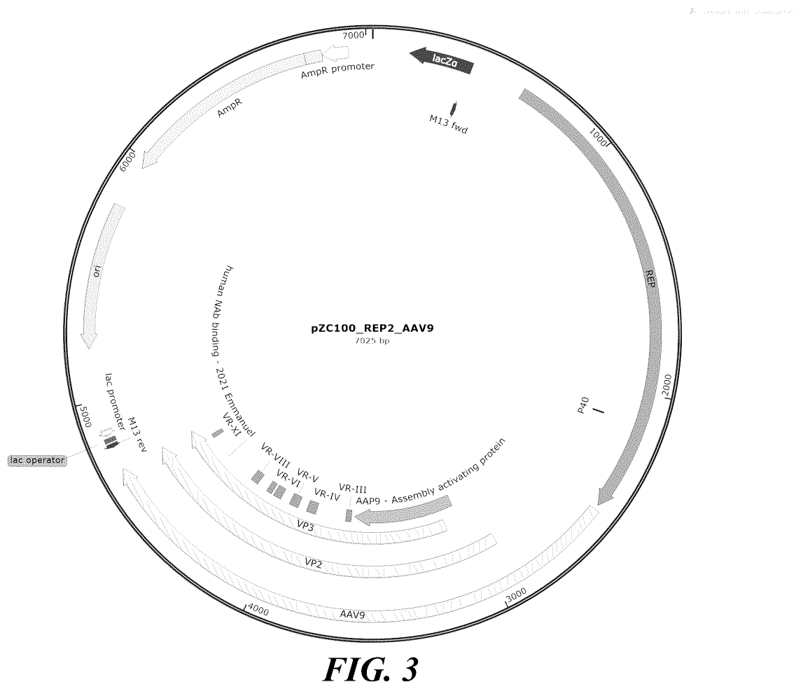

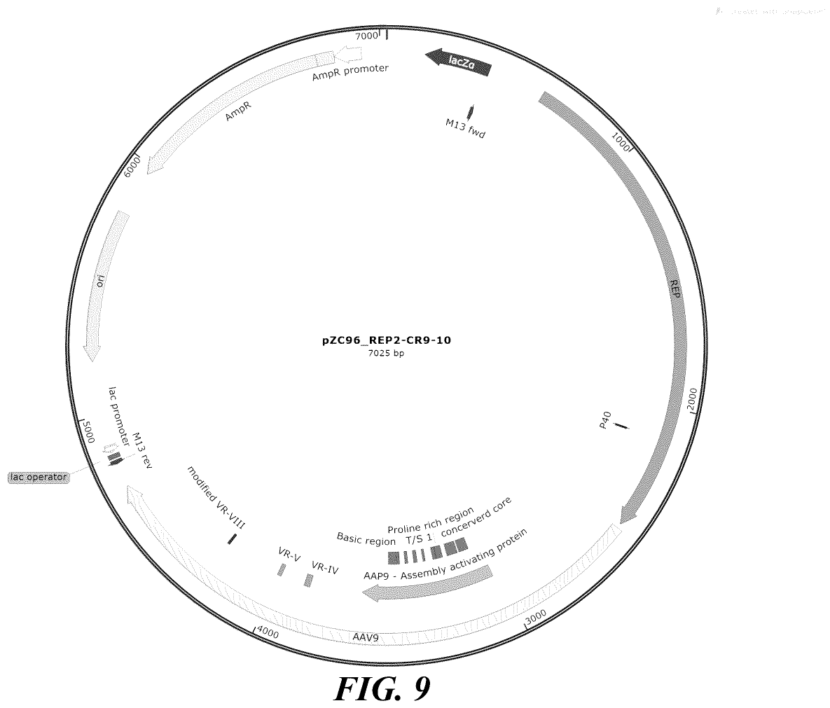

When cell viability reached >90%, 3 days post-seeding, transfection of a vector with a transgene, a Rep/Cap plasmid (a plasmid with AAV replication protein and a capsid protein), and a helper plasmid was performed, using PEI (polyethyleneimine)-based transfection reagent. The viable cell density at the time of transfection was 2E+6 cells/mL. The drawings presented herein show the maps of the vector with a transgene (FIG. 1), a helper plasmid (Adhelper) (FIG. 2), and Rep/Cap plasmids (FIGS. 3-9) used. The sequences of these vectors/plasmids are provided in the Sequence Table hereinbelow.

HDAC6 inhibitors diluted in DMSO at stock concentrations of 0.25 mM to 1 mM. The stock solutions were then added to the cell culture on the day of the transfection at a working concentration between 0.1 μM to 100 μM (the specific concentrations used are indicated in the tables below).

The cells were harvested 3 days post transfection for AAV purification.

### Experimental Results

The experimental results show that the selective HDAC6 inhibitors tested significantly improved viral production, and in particular, increased viral titer and yield in mammalian cells, in particular, in HEK293/IEK293T cells. The experimental results also show that other HDAC6 inhibitors, such as pan-HDAC inhibitors, did not improve viral production as much as the selective HDAC6 inhibitors described herein.

### Experiment #1

Summary: Compound I-6 of Table 3 enhanced AAV9 yield in Expi-293 cell culture, cultivated in serum-free medium, at resulting concentrations (in the medium) of 1.25 μM and 2.50 μM.

Experiment #1

| | | | | | | | No Enhancer | |
|---|---|---|---|---|---|---|---|---|
| | | | | | | | Control | % Titer |
| | | Cell Culture | | | Enhancer | | Titer | Improvement |
| AAV Serotype | Cells | Medium | Duplicate | Enhancer | Conc (uM) | Titer (vg/mL) | (vg/mL) | over Control |
| AAV9 | Expi- | Serum- | #1 | I-6 | 1.25 | 2.37E+11 | 1.72E+11 | 38% |
| | 293 | free | | | | | | |
| AAV9 | Expi- | Serum- | #2 | I-6 | 1.25 | 2.27E+11 | 1.72E+11 | 32% |
| | 293 | free | | | | | | |
| AAV9 | Expi- | Serum- | #1 | I-6 | 2.50 | 2.46E+11 | 1.72E+11 | 43% |
| | 293 | free | | | | | | |
| AAV9 | Expi- | Serum- | #2 | I-6 | 2.50 | 2.47E+11 | 1.72E+11 | 44% |
| | 293 | free | | | | | | |

### Experiment #2

Summary: Compound I-6 of Table 3 enhanced 4 different AAV9 variants, yield in Expi-293 cell culture, cultivated in serum-free medium, at concentration of 1.25 μM

Experiment #2

| | | | | | | | No | % |
|---|---|---|---|---|---|---|---|---|
| | | | | | | | Enhancer | Titer |
| | | Cell | | | Enhancer | | Control | Improvement |
| AAV | | Culture | | | Conc | Titer | Titer | over |
| Serotype | Cells | Medium | Duplicate | Enhancer | (uM) | (vg/mL) | (vg/mL) | Control |
| AAV9 | Expi- | Serum- | #1 | I-6 | 1.25 | 1.59E+11 | 1.22E+11 | 30% |
| Variant | 293 | free | | | | | | |
| #1=pZC401 | | | | | | | | |
| AAV9 | Expi- | Serum- | #1 | I-6 | 1.25 | 5.98E+10 | 4.45E+10 | 34% |
| Variant | 293 | free | | | | | | |
| #2=pZC428 | | | | | | | | |
| AAV9 | Expi- | Serum- | #1 | I-6 | 1.25 | 8.84E+10 | 4.01E+10 | 120% |
| Variant | 293 | free | | | | | | |
| #3=pZC478 | | | | | | | | |
| AAV9 | Expi- | Serum- | #1 | I-6 | 1.25 | 5.36E+10 | 3.92E+10 | 37% |
| Variant | 293 | free | | | | | | |
| #4=pZC375 | | | | | | | | |

### Experiment #3

Summary: Compounds I-6, I-3, and TYA of Table 3 enhanced AAV9 yield in Expi 293 culture, cultivated in chemically defined medium, at concentrations of 1.25 μM. Neither sodium butyrate nor givinostat exhibited the improvement demonstrated by the selective HDAC6 inhibitors described herein. TYA is a compound within Formula (I), as well as within, for example, Formula (Ic), Formula (Ik) and Formula I(y). Reference to TYA below references the same compound.

Experiment #3

| | | | | | | | No | % |
|---|---|---|---|---|---|---|---|---|
| | | | | | | | Enhancer | Titer |
| | | Cell | | | Enhancer | | Control | Improvement |
| AAV | | Culture | | | Conc | Titer | Titer | over |
| Serotype | Cells | Medium | Duplicate | Enhancer | (uM) | (vg/mL) | (vg/mL) | Control |
| AAV9 | Expi- | Serum- | #1 | I-6 | 1.25 | 1.6E+11 | 1.08E+11 | 48% |
| | 293 | free | | | | | | |
| AAV9 | Expi- | Serum- | #1 | I-3 | 1.25 | 1.59E+11 | 1.08E+11 | 47% |
| | 293 | free | | | | | | |
| AAV9 | Expi- | Serum- | #1 | TYA | 1.25 | 1.66E+11 | 1.08E+11 | 54% |
| | 293 | free | | | | | | |
| AAV9 | Expi- | Serum- | #1 | Sodium | 1.25 | 0.93E+10 | 1.08E+11 | −14% |
| | 293 | free | | Butyrate | | | | |
| AAV9 | Expi- | Serum- | #1 | Givinostat | 1.25 | 1.21E+11 | 1.08E+11 | +11% |
| | 293 | free | | | | | | |

### Experiment #4

Summary: Compound I-6 of Table 3 enhanced AAV9 yield in VPC 2.0 HEK293 culture, cultivated in chemically defined medium, at concentrations of 0.50 μM, 1.25 μM, 2.25 μM, and 5.00 μM.

Experiment #4

| | | | | | | | No | % |
|---|---|---|---|---|---|---|---|---|
| | | | | | | | Enhancer | Titer |
| | | Cell | | | Enhancer | | Control | Improvement |
| AAV | | Culture | | | Conc | Titer | Titer | over |
| Serotype | Cells | Medium | Duplicate | Enhancer | (uM) | (vg/mL) | (vg/mL) | Control |
| AAV9 | VPC 2.0 | Chemically | #1 | I-6 | 0.50 | 2.28E+11 | 1.57E+11 | 45% |
| | HEK293 | Defined | | | | | | |
| AAV9 | VPC 2.0 | Chemically | #2 | I-6 | 0.50 | 2.49E+11 | 1.9E+11 | 31% |
| | HEK293 | Defined | | | | | | |
| AAV9 | VPC 2.0 | Chemically | #1 | I-6 | 1.25 | 2.51E+11 | 1.57E+11 | 60% |
| | HEK293 | Defined | | | | | | |
| AAV9 | VPC 2.0 | Chemically | #2 | I-6 | 1.25 | 3.01E+11 | 1.9E+11 | 58% |
| | HEK293 | Defined | | | | | | |
| AAV9 | VPC 2.0 | Chemically | #1 | I-6 | 2.50 | 2.3E+11 | 1.57E+11 | 46% |
| | HEK293 | Defined | | | | | | |
| AAV9 | VPC 2.0 | Chemically | #2 | I-6 | 2.50 | 2.65E+11 | 1.9E+11 | 39% |
| | HEK293 | Defined | | | | | | |
| AAV9 | VPC 2.0 | Chemically | #1 | I-6 | 5.00 | 2.4E+11 | 1.57E+11 | 53% |
| | HEK293 | Defined | | | | | | |
| AAV9 | VPC 2.0 | Chemically | #2 | I-6 | 5.00 | 2.59E+11 | 1.9E+11 | 36% |
| | HEK293 | Defined | | | | | | |

### Experiment #5

Summary: Compounds I-3 and TYA of Table 3 enhanced AAV yield in VPC 2.0 HTEK293 culture, cultivated in chemically defined medium, at concentrations of 0.01 μM, 0.10 μM, 0.50 μM, and 1.25 μM, respectively.

Experiment #5

| | | | | | | | No | % |
|---|---|---|---|---|---|---|---|---|
| | | | | | | | Enhancer | Titer |
| | | Cell | | | Enhancer | | Control | Improvement |
| AAV | | Culture | | | Conc | Titer | Titer | over |
| Serotype | Cells | Medium | Duplicate | Enhancer | (uM) | (vg/mL) | (vg/mL) | Control |
| AAV9 | VPC 2.0 | Chemically | #1 | I-3 | 0.01 | 1.16E+11 | 9.01E+10 | 29% |
| | HEK293 | Defined | | | | | | |
| AAV9 | VPC 2.0 | Chemically | #1 | I-3 | 0.10 | 1.33E+11 | 9.01E+10 | 48% |
| | HEK293 | Defined | | | | | | |
| AAV9 | VPC 2.0 | Chemically | #1 | I-3 | 0.50 | 1.25E+11 | 9.01E+10 | 39% |
| | HEK293 | Defined | | | | | | |
| AAV9 | VPC 2.0 | Chemically | #1 | I-3 | 1.25 | 1.41E+11 | 9.01E+10 | 56% |
| | HEK293 | Defined | | | | | | |
| AAV9 | VPC 2.0 | Chemically | #1 | TYA | 0.01 | 1.09E+11 | 9.01E+10 | 21% |
| | HEK293 | Defined | | | | | | |
| AAV9 | VPC 2.0 | Chemically | #1 | TYA | 0.10 | 1.34E+11 | 9.01E+10 | 49% |
| | HEK293 | Defined | | | | | | |
| AAV9 | VPC 2.0 | Chemically | #1 | TYA | 0.50 | 1.38E+11 | 9.01E+10 | 53% |
| | HEK293 | Defined | | | | | | |
| AAV9 | VPC 2.0 | Chemically | #1 | TYA | 1.25 | 1.59E+11 | 9.01E+10 | 76% |
| | HEK293 | Defined | | | | | | |

### Experiment #6

Summary: Compounds I-6 and TYA of Table 3 enhanced AAV9 variant #5 yield in VPC 2.0 HEK293 cells, cultivated in chemically defined medium at concentration of 1.25 μM and 2.50 μM respectively.

Experiment #6

| | | | | | | | No | % |
|---|---|---|---|---|---|---|---|---|
| | | | | | | | Enhancer | Titer |
| | | Cell | | | Enhancer | | Control | Improvement |
| AAV | | Culture | | | Conc | Titer | Titer | over |
| Serotype | Cells | Medium | Duplicate | Enhancer | (uM) | (vg/mL) | (vg/mL) | Control |
| AAV9 | VPC 2.0 | Chemically | #1 | I-6 | 1.25 | 9.70E+10 | 6.99E+10 | 39% |
| Variant | HEK293 | Defined | | | | | | |
| #5=pZC96 | | | | | | | | |
| AAV9 | VPC 2.0 | Chemically | #1 | I-6 | 2.50 | 8.13E+10 | 6.99E+10 | 16% |
| Variant | HEK293 | Defined | | | | | | |
| #5=pZC96 | | | | | | | | |
| AAV9 | VPC 2.0 | Chemically | #1 | TYA | 1.25 | 8.56E+10 | 6.99E+10 | 22% |
| Variant | HEK293 | Defined | | | | | | |
| #5=pZC96 | | | | | | | | |
| AAV9 | | | | | | | | |
| Variant | VPC 2.0 | Chemically | | | | | | |
| #5=pZC96 | HEK293 | Defined | #1 | TYA | 2.50 | 8.98E+10 | 6.99E+10 | 28% |

### Experiment #7

### Summary:

Compounds I-6 and I-3 of Table 3 enhanced AAV9 yield in VPC 2.0 HEK293 cell culture, cultivated in chemically defined medium at concentration of 1.25 μM

Compound TYA of Table 3 enhanced AAV9 yield in VPC 2.0 HEK293 cell culture, cultivated in chemically defined medium at concentrations of 1.25 μM, 2.50 μM, 5.00 M, and 10.00 μM.

Experiment #7

| | | | | | | | No | % |
|---|---|---|---|---|---|---|---|---|
| | | | | | | | Enhancer | Titer |
| | | Cell | | | Enhancer | | Control | Improvement |
| AAV | | Culture | | | Conc | Titer | Titer | over |
| Serotype | Cells | Medium | Duplicate | Enhancer | (uM) | (vg/mL) | (vg/mL) | Control |
| AAV9 | VPC 2.0 | Chemically | #1 | I-6 | 1.25 | 2.32E+11 | 1.71E+11 | 36% |
| | HEK293 | Defined | | | | | | |
| AAV9 | VPC 2.0 | Chemically | #1 | I-3 | 1.25 | 2.49E+11 | 1.71E+11 | 46% |
| | HEK293 | Defined | | | | | | |
| AAV9 | VPC 2.0 | Chemically | #1 | TYA | 1.25 | 2.40E+11 | 1.71E+11 | 40% |
| | HEK293 | Defined | | | | | | |
| AAV9 | VPC 2.0 | Chemically | #1 | TYA | 2.50 | 2.65E+11 | 1.71E+11 | 55% |
| | HEK293 | Defined | | | | | | |
| AAV9 | VPC 2.0 | Chemically | #1 | TYA | 5.00 | 2.20E+11 | 1.71E+11 | 29% |
| | HEK293 | Defined | | | | | | |
| AAV9 | VPC 2.0 | Chemically | #1 | TYA | 10.00 | 2.13E+11 | 1.71E+11 | 25% |
| | HEK293 | Defined | | | | | | |

### Experiment #8

Summary: Compounds I-6, I-3, and TYA of Table 3 enhanced AAV9 yield in VPC 2.0 HTEK293, cultivated in chemically defined medium, at concentration of 1.25 μM. Neither sodium butyrate nor givinostat exhibited the improvement demonstrated by the selective HDAC6 inhibitors described herein.

Experiment #8

| | | | | | | | No | % |
|---|---|---|---|---|---|---|---|---|
| | | | | | | | Enhancer | Titer |
| | | Cell | | | Enhancer | | Control | Improvement |
| AAV | | Culture | | | Conc | Titer | Titer | over |
| Serotype | Cells | Medium | Duplicate | Enhancer | (uM) | (vg/mL) | (vg/mL) | Control |
| AAV5 | VPC 2.0 | Chemically | #1 | I-6 | 1.25 | 2.09E+11 | 1.04E+11 | 101% |
| | HEK293 | Defined | | | | | | |
| AAV5 | VPC 2.0 | Chemically | #1 | I-3 | 1.25 | 2.19E+11 | 1.04E+11 | 111% |
| | HEK293 | Defined | | | | | | |
| AAV5 | VPC 2.0 | Chemically | #1 | TYA | 1.25 | 1.97E+11 | 1.04E+11 | 89% |
| | HEK293 | Defined | | | | | | |
| AAV5 | VPC 2.0 | Chemically | #1 | Sodium | 1.25 | 1.01E+11 | 1.04E+11 | −3% |
| | HEK293 | Defined | | Butyrate | | | | |
| AAV5 | VPC 2.0 | Chemically | #1 | Givinostat | 1.25 | 1.23E+11 | 1.04+E11 | +18% |
| | HEK293 | Defined | | | | | | |

### Experiment #9

Summary: Compounds I-6, I-3, and TYA of Table 3 enhanced AAV yield in ATCC 1573 HTEK293 cell culture, cultivated in fetal bovine serum (FBS) containing medium, at concentration of 1.25 μM.

Experiment #9

| | | | | | | | No | % |
|---|---|---|---|---|---|---|---|---|
| | | | | | | | Enhancer | Titer |
| | | | | | Enhancer | | Control | Improvement |
| AAV | | | | | Conc | Titer | Titer | over |
| Serotype | Cells | Medium | Duplicate | Enhancer | (uM) | (vg/mL) | (vg/mL) | Control |
| AAV9 | ATCC | FBS | #1 | I-6 | 1.25 | 9.52E+04 | 1.00E+03 | 9420% |
| | 1573 | Containing | | | | | | |
| | HEK | | | | | | | |
| AAV9 | ATCC | FBS | #1 | I-3 | 1.25 | 1.23E+05 | 1.00E+03 | 12200% |
| | 1573 | Containing | | | | | | |
| | HEK | | | | | | | |
| AAV9 | ATCC | FBS | #1 | TYA | 1.25 | 1.01E+05 | 1.00E+03 | 10000% |
| | 1573 | Containing | | | | | | |
| | HEK | | | | | | | |

### Experiment #10

Summary: Compounds I-6, I-3, and TYA of Table 3 enhanced AAV yield in ATCC 3216 HTEK293T cell culture, cultivated in fetal bovine serum (FBS) containing medium, at concentration of 1.25 μM.

Experiment #10

| | | | | | | | No | % |
|---|---|---|---|---|---|---|---|---|
| | | | | | | | Enhancer | Titer |
| | | | | | Enhancer | | Control | Improvement |
| AAV | | | | | Conc | Titer | Titer | over |
| Serotype | Cells | Medium | Duplicate | Enhancer | (uM) | (vg/mL) | (vg/mL) | Control |
| AAV9 | ATCC | FBS | #1 | I-6 | 1.25 | 6.56E+10 | 4.85E+10 | 35% |
| | 3216 | Containing | | | | | | |
| | HEK293T | | | | | | | |
| AAV9 | ATCC | FBS | #1 | I-3 | 1.25 | 7.02E+10 | 4.85E+10 | 45% |
| | 3216 | Containing | | | | | | |
| | HEK293T | | | | | | | |
| AAV9 | ATCC | FBS | #1 | I-96 | 1.25 | 6.11E+10 | 4.85E+10 | 26% |
| | 3216 | Containing | | | | | | |
| | HEK293T | | | | | | | |

### Experiment #11

Summary: Compound I-6 of Table 3 enhanced AAV9 yield in Expi-293 culture, cultivated in serum-free medium in 3L and 50 L bioreactors, at a concentration of 1.25 μM.

Experiment #11

| | | | | | | | No | % |
|---|---|---|---|---|---|---|---|---|
| | | | | | | | Enhancer | Titer |
| | | | | | Enhancer | | Control | Improvement |
| AAV | | | Bioreactor | | Conc | Titer | Titer | over |
| Serotype | Cells | Medium | Scale | Enhancer | (uM) | (vg/mL) | (vg/mL) | Control |
| AAV9 | Expi- | Serum- | 3 L | I-6 | 1.25 | 1.8 | 1.1 | 64% |
| | 239 | free | | | | | | |
| AAV9 | Expi- | Serum- | 50 L | I-6 | 1.25 | 3.8 | 1.1 | 245% |
| | 239 | free | | | | | | |

### Example 4. Titer Boosting and Scaling-Up of HEK293-Based AAV Production by HDAC6 Inhibitors

The main limitation of viral production in HEK293 cell culture is scalability and viral productivity. This example shows that selective HDAC6 inhibitors described herein (also referred to as small molecule boosters (SMBs) in this example), including N-((5-(5-(difluoromethyl)-1,3,4-oxadiazol-2-yl) thiazol-2-yl)methyl)-N-(pyridin-3-yl) ethanesulfonamide (Compound I-6, see Table 3), can significantly increase AAV yield in a cell line and cell culture media-independent manner. Additionally, this example shows that these compounds can enhance the scalability of HEK293-process by maintaining consistent yield at least up to 200 L. Further, this example shows that these compounds have no impact on purity, quality, safety, and potency of the AAV viral vector and can be readily cleared in standard AAV purification process, suggesting that they can potentially be transformational in debottlenecking AAV manufacturing and decreasing of cost of AAV gene therapy.

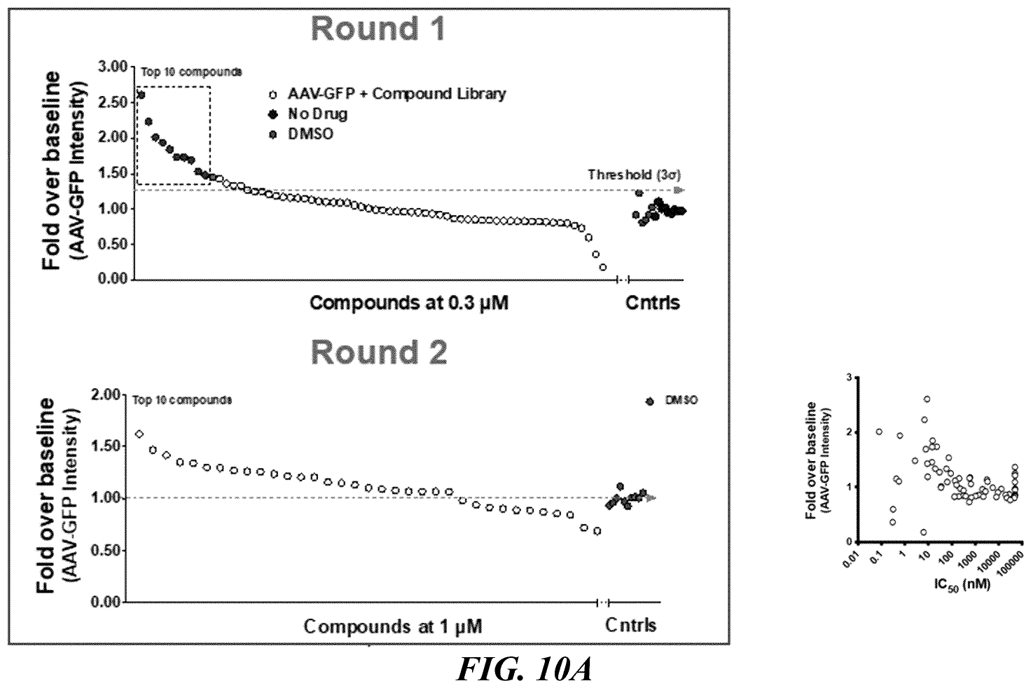

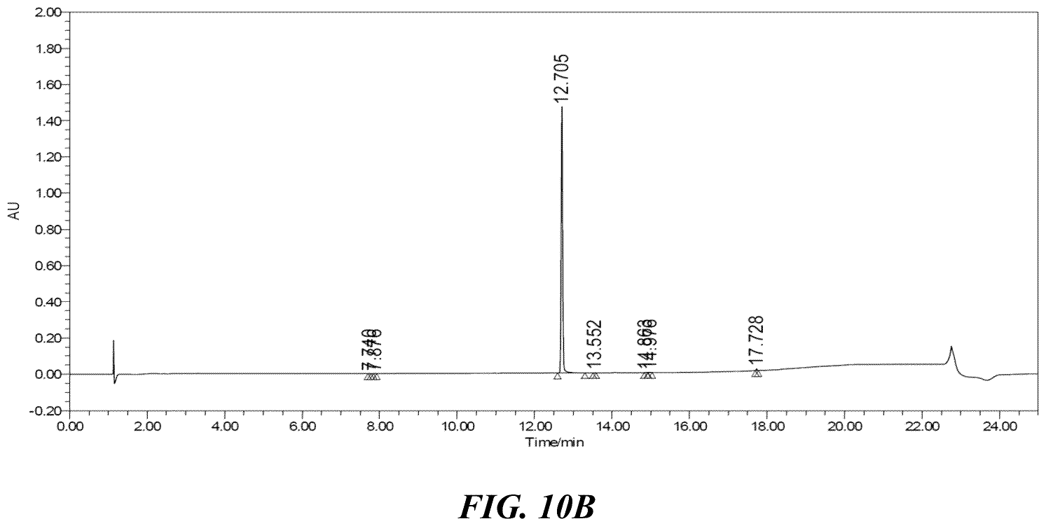

First, the selective HDAC6 inhibitors described herein, including N-((5-(5-(difluoromethyl)-1,3,4-oxadiazol-2-yl)thiazol-2-yl)methyl)-N-(pyridin-3-yl)ethanesulfonamide (Compound I-6, see Table 3), were screened against other compounds by AAV-GFP intensity, as measured in each culture to which a test compound was added (FIGS. 10A-10B).

The materials and methods used in the experiments described herein are consistent with those described in Example 3.

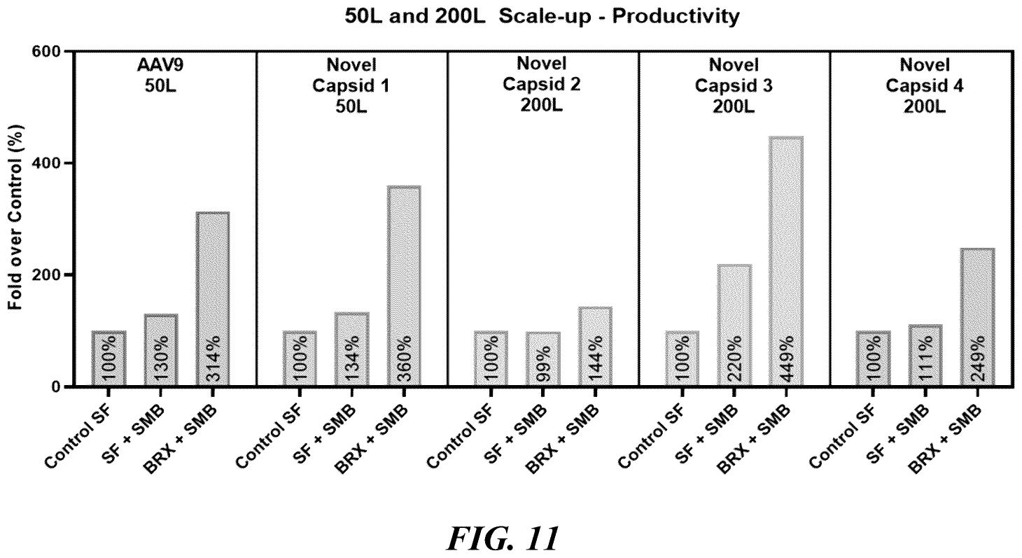

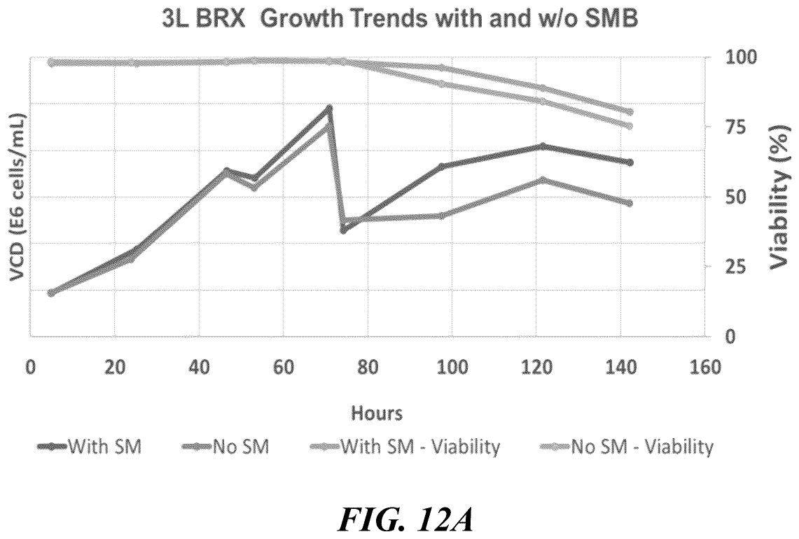

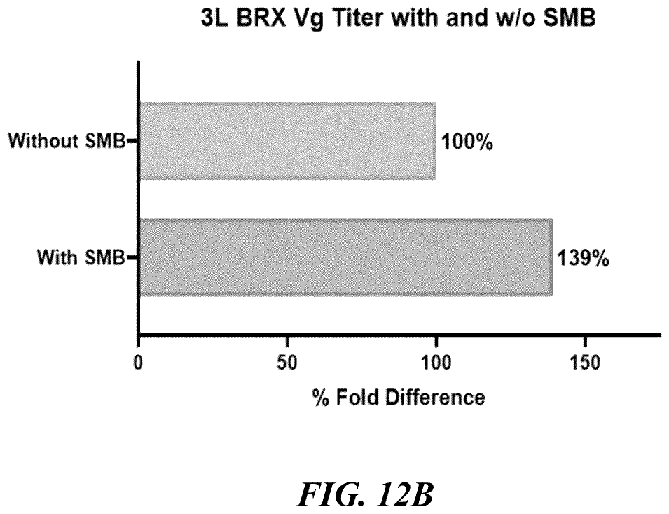

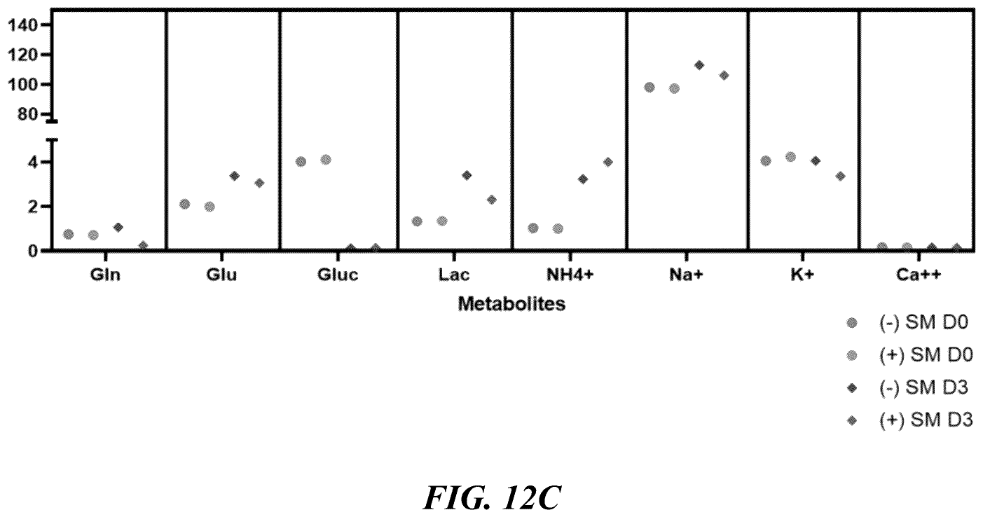

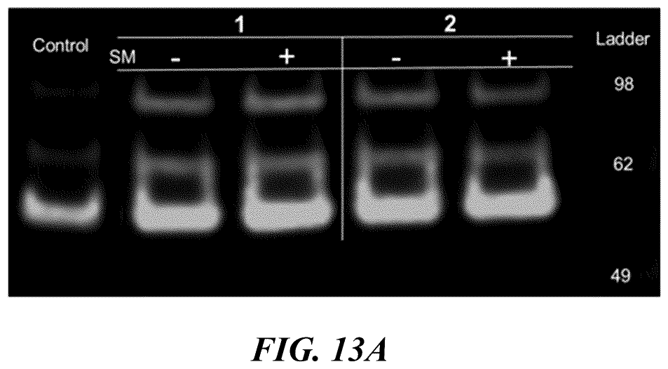

To test for improvement of the scalability of HEK293 process, selective HDAC6 inhibitor compounds were tested in shake flasks. In particular, Compound I-6 showed improved yield, titer, and productivity at 3 L, 50 L, and 200 L bioreactor (BRX) scale based on power per unit volume (PV) (FIGS. 11, 12A-12C), showing capability to enhance the scalability and productivity of the AAV manufacturing process on at least up to 200 L BRX scale.

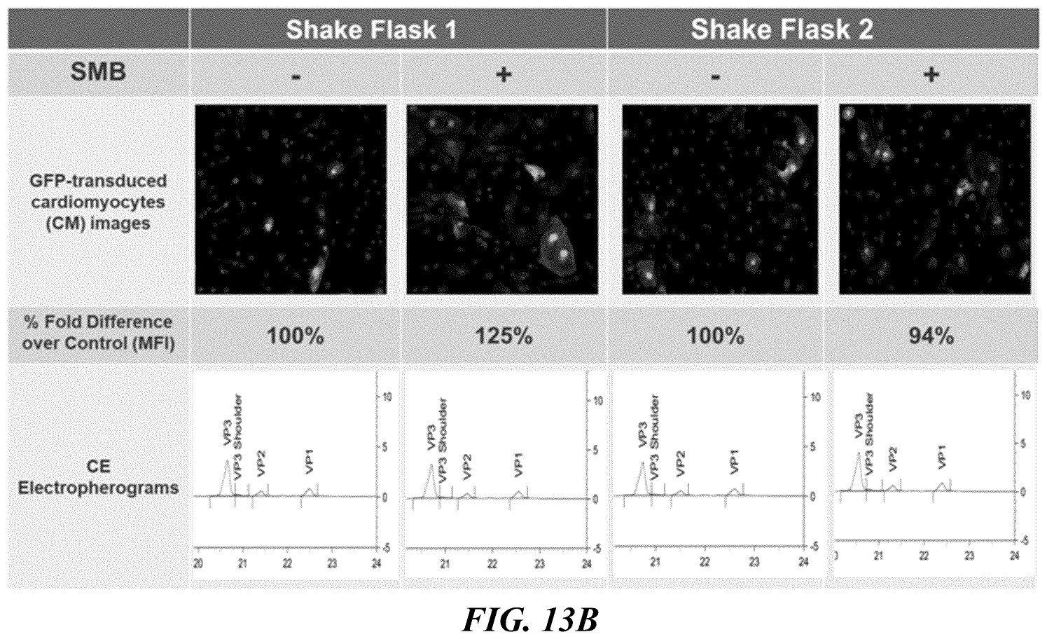

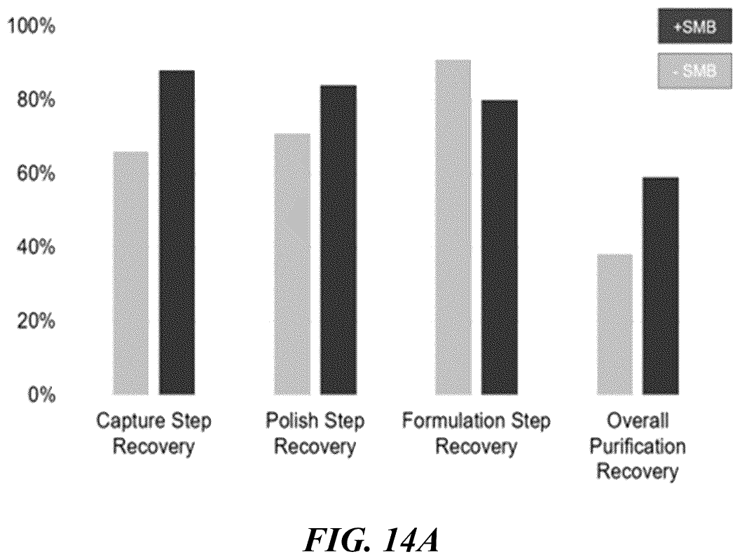

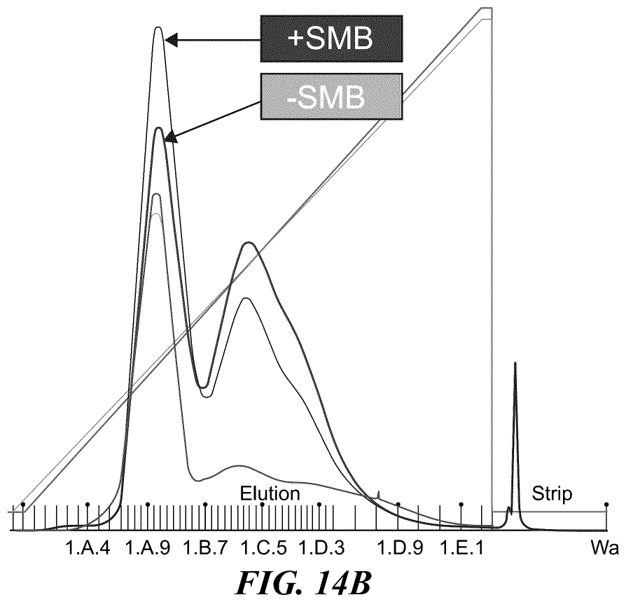

Next, it was tested whether addition of selective HDAC6 inhibitor compounds to cell culture affected product quality or purification performance. FIG. 13A shows Western blot of affinity purified AAVs from small scale shake flask productions (2 replicates, one with addition of Compound I-6 and one without). Consistent ratios of VP1/VP2/VP3 with and without a selective HDAC6 inhibitor addition were observed (Compound I-6 was used in the experiment shown here). Based on cardiomyocyte transduction with AAV9:GFP (FIG. 13B), there was no significant impact of Compound I-6 addition on AAV infectivity. Additionally, from the capillary electrophoresis (CE) electropherograms, VP1/VP2/VP3 ratios were found to be similar when produced in cultures with Compound I-6 added versus control (no compound added), suggesting that there was no impact on product quality. Additionally, as shown in FIGS. 14A-14B, there was also no impact of selective HDAC6 inhibitor addition on purification performance.

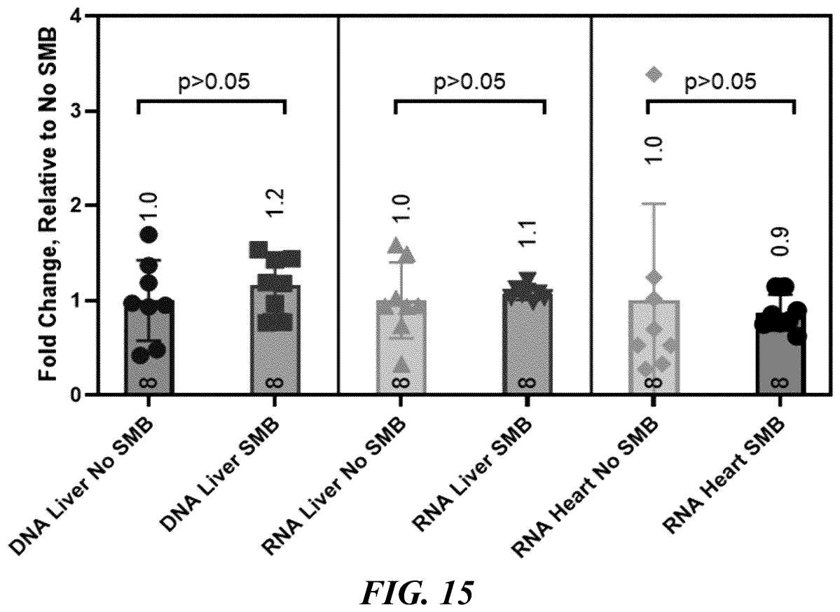

Next, as shown in FIG. 15, a selective HDAC6 inhibitor also had no impact on in vivo transduction using AAV. The in vivo study consisted of two arms, the first injected with AAV produced without a selective HDAC6 inhibitor and the second injected with AAV produced with a selective HDAC6 inhibitor (Compound I-6 was used in the experiment shown here). Mice were euthanized 3 weeks post injection and harvested for heart and liver tissue, which were extracted for RNA and DNA and analyzed using qPCR to determine fold change in gene of interest (GOI) relative to a process without a selective HDAC6 inhibitor added to the media. No significant differences were seen in the mouse study for viral production with or without a selective HDAC6 inhibitor.

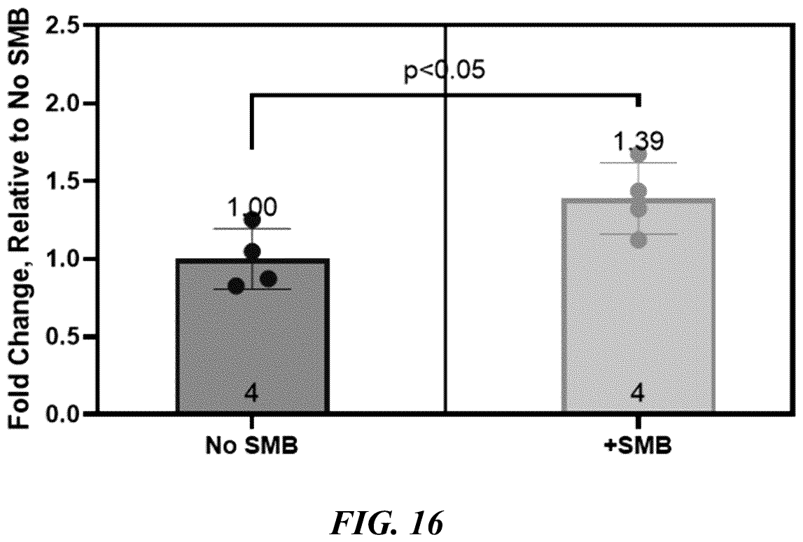

Moreover, a selective HDAC6 inhibitor has been shown to increase viral titer without impacting quality of product produced. As shown in FIG. 16, lentiviral production with a selective HDAC6 inhibitor (Compound I-6 was used in the experiment shown here) increased titer by >39% over a process without a selective HDAC6 inhibitor, showing that the selective HDAC6 inhibitor compounds described herein may be effective in increasing viral production of other vector types such as lentivirus.

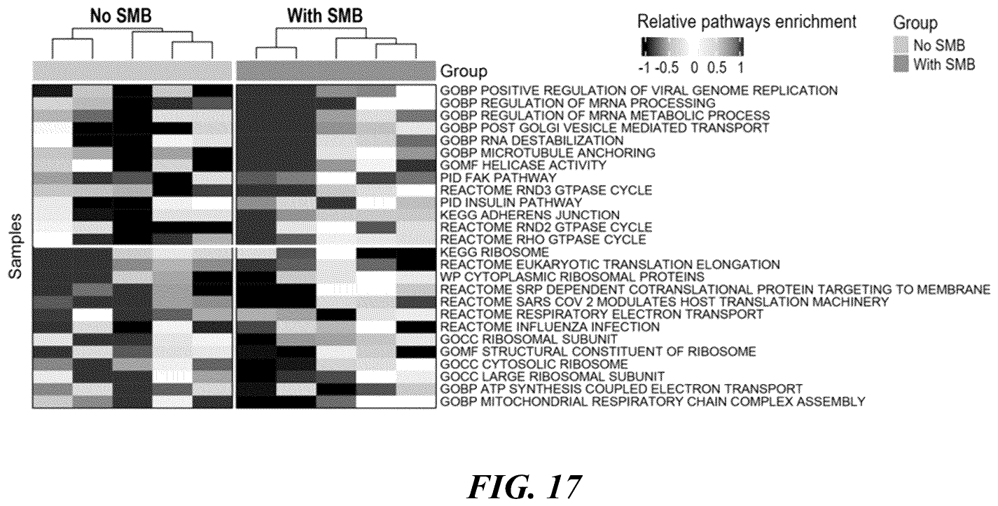

Finally, as shown in FIG. 17, the selective HDAC6 inhibitor compounds described herein demonstrate unique mechanisms of action that affect many different pathways during the production of AAV in HEL293 cells (Compound I-6 was used in the experiment shown here).

Overall, the data presented in this example show that selective HDAC6 inhibitors described herein (such as Compound I-6) can plug-and-play into any existing HEK-based process and consistently show a substantial increase in viral productivity. The observed titer improvements were media, cell-line, and scale independent, and the addition of a selective HDAC6 inhibitor had no observable impact on product quality, purity, and transduction efficiency, both in vitro and in vivo.

### Example 5. Titer Boosting of HEK293-Based AAV Production by Combinations of Inhibitors

To further increase viral productivity, selective HDAC6 inhibitors described herein were tested in combination with other compounds (collectively also referred to as small molecule boosters (SMBs) in this example). In this example, the selective HDAC6 inhibitor N-((5-(5-(difluoromethyl)-1,3,4-oxadiazol-2-yl)thiazol-2-yl)methyl)-N-(pyridin-3-yl)ethanesulfonamide (Compound I-6, see Table 3, also referred to as TYA-004 in this example) was tested in combination with (1) Ac-DEVD-CHO (N—Ac-Asp-Glu-Val-Asp-CHO), a caspases-3 and caspase-7 inhibitor; (2)SN-011, a selective inhibitor of stimulator of interferon genes (STING), an integral ER-membrane protein; and/or (3) Ricolinostat, a pan-HDAC inhibitor of HDAC1, HDAC2, and HDAC3, as well as a potent and selective HDAC6 inhibitor. This example shows that selected HDAC6 inhibitors can significantly increase AAV yield in HEK293 cells when used in combination with STING inhibitors, caspase inhibitors, and pan-HDAC inhibitors.

### Materials and Methods

HEK293 Cell Cultures in Shake Flasks and Triple Transient Transfection for AAV Production in HEK293 cells

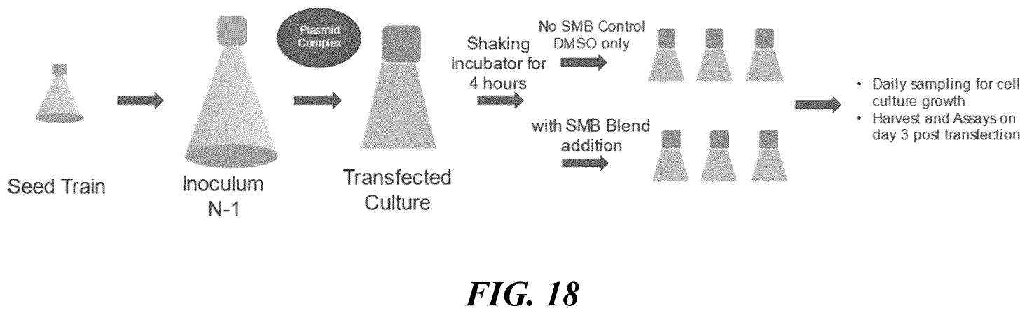

As illustrated in FIG. 18, the viable cell density (VCD) of HEK293 cells was determined using Vi-CELL XR (Beckman Coulter), and HEK293 cells were seeded at 0.6-0.7E+6 cells/mL into fresh, pre-warmed HEK ViP NX media (Sartorius; #892-0001) supplemented with GlutaMAX (Gibco; #35050061) in a shake flask. The pH for cell culture media in this example is about 7.4 unless noted otherwise. The ratio of media volume to flask size used was 30:125. For example, 240 mL of supplemented HEK ViP NX media was used in a 1 L flask. The flasks were incubated at 37° C. incubator with ≥80% relative humidity and 8% CO₂ in an orbital shaker platform until the cultures reached a density of 4-6×E+6 cells/mL. The VCD of the HEK293 cultures was determined as described above 3 days post-seeding. The appropriate volume of plasmids (for a final ratio of 1 μg DNA/million cells) were thawed, vortexed briefly, and added to a predetermined amount of prewarmed high glucose Dulbecco's Modified Eagle Medium (DMEM; Gibco; Ser. No. 10/566,024). For each 30 mL of final culture volume, 1.5 mL (5% of the volume of culture volume) of high glucose DMEM was used. FectoVIR-AAV (Polyplus; #101000004) was vortexed briefly and added to the diluted plasmid DNA/DMEM solution to prepare a transfection complex solution with a final transfection ratio of 1 μg DNA:1 μl FectoVIR-AAV. The transfection complex solution was vortexed briefly and incubated for 30 minutes at room temperature. HEK293 cells were then diluted to 2.5-3E+6 cells/mL and the transfection complex solution was added onto the cells. The flasks were incubated in a 37° C. incubator with 80% relative humidity and 8% CO₂ in an orbital shaker platform for 4 hours. 4 hours post transfection, dimethyl sulfoxide (DMSO; Sigma; #D2438) only or different small molecules suspended in DMSO were added such that the final concentrations of TYA-004, Ac-DEVD-CHO (Cas No. 169332-60-9), SN-011 (Cas No. 2249435-90-1), and Ricolinostat (Cas No. 1316214-52-4) were 1.25 μM, 2.50 μM, 1.25 μM, and 1.25 μM, respectively.

### AAV Production Harvest

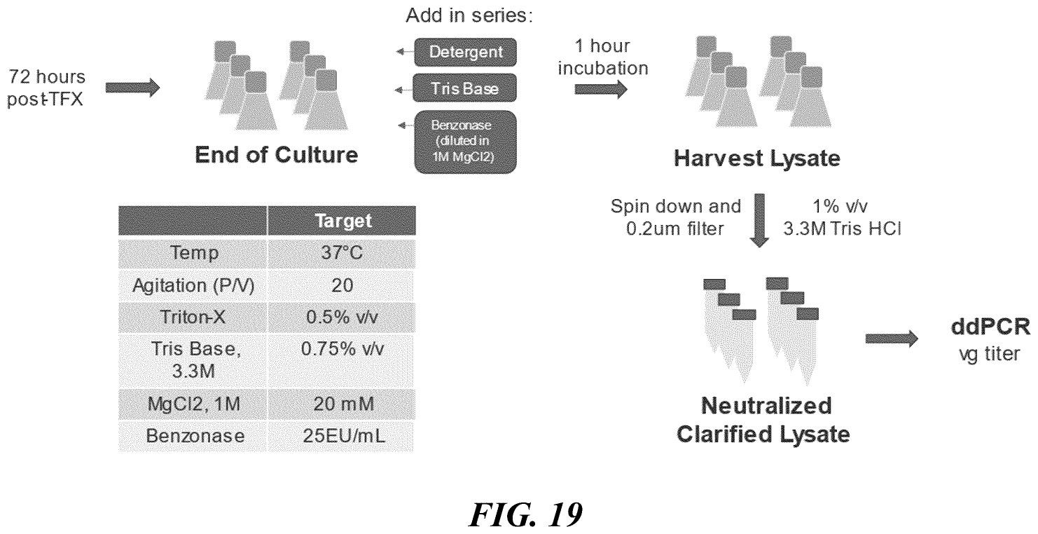

As shown in FIG. 19, the VCD was determined for the transfected cultures 68-72 hours post-transfection and Triton X-100, tris base (3.3 M), MgCl₂ (1 M), and benzonase were added at final concentrations of 0.5% v/v, 0.75% v/v, 20 mM, and 25 EU/mL, respectively, to start the lysis process. For example, 150 μL of Triton X-100, 225 μL of tris base (3.3 M), 60 μL of MgCl₂ (1 M), and 3 μL of benzonase were added to 30 mL of transfected cultures. The resulting lysates were incubated for 1 hour at 37° C. in the orbital shaker incubator. After incubation, 1% v/v of 3.3 M tris HCl (300 μL for 30 mL culture) was added. The lysates were centrifuged at 4000 g for 10 minutes and passed through a 0.2 μm filter (ThermoFisher). A sample was taken for ddPCR analysis to determine AAV viral titer.

### Experimental Results

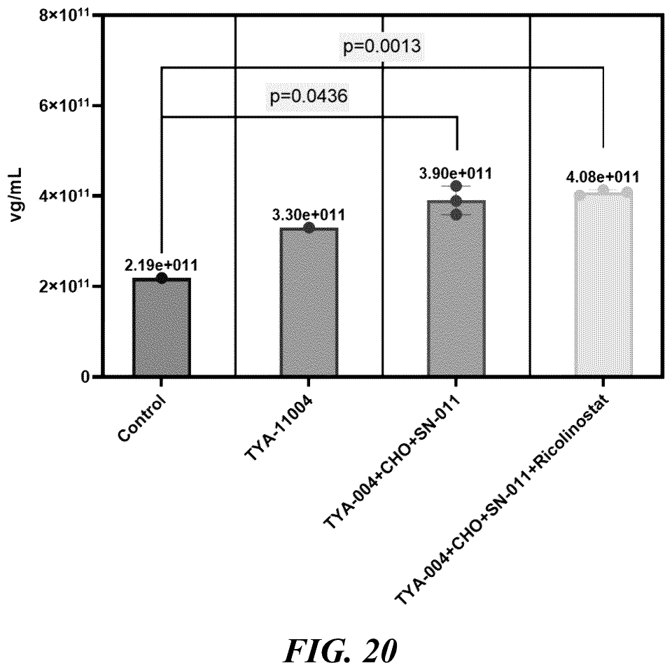

As shown in FIG. 20, a viral titer of 2.19E+11 vg/mL was measured in the DMSO control culture. Addition of TYA-004 alone to the transfected culture yielded a significantly higher AAV viral titer of 3.30×E+11 vg/mL. Importantly, a combination of TYA-004, Ac-DEVD-CHO, and SN-011 (TYA-004+CHO+SN-011) and a combination of TYA-004, Ac-DEVD-CHO, SN-011, and Ricolinostat (TYA-004+CHO+SN-011+Ricolinostat) further increased AAV viral titer compared to control and TYA-004 alone. These results show that the selective HDAC6 inhibitors of the present technology can be used in combination with other compounds as described to further improve viral productivity in HEK293 cells.

TABLE 6
Selective Sequences

| | | SEQ |
|---|---|---|
| | | ID |
| Description | Sequence | NO: |
| A viral vector | gcccttaagtcatggagaagacccaccttgcagatgtcct | 1 |
| carrying a | cactggggctggcagagccggcaacctgcccaaggctgct | |
| transgene: | cagtccattaggagccagtagcctggaagatgtctttacc | |
| jy08-paav2-1- | cccagcatcagttcaagtggagcagcacataactcttgcc | |
| 2nd- | ctctgccttccaagattctggtgctgagacttatggagtg | |
| gen_ptnt600- | tcttggaggttgccttctgccccccaaccctgctcccagc | |
| hpkp2- | tggccctcccaggcctgggttgctggcctctgctttatca | |
| optimized-wpre | ggattctcaagagggacagctggtttatgttgcatgactg | |
| | ttccctgcatatctgctctggttttaaatagcttatctga | |
| | gcagctggaggaccacatgggcttatatggcgtggggtac | |
| | atgttcctgtagccttgtccctggcacctgccaaaatagc | |
| | agccaacaccccccacccccaccgccatccccctgcccca | |
| | cccgtcccctgtcgcacattcctccctccgcagggctggc | |
| | tcaccaggccccagcccacatgcctgcttaaagccctctc | |
| | catcctctgcctcacccagtccccgctgagactgagcaga | |
| | cgcctccagccaccatggctgctcctggtgctcctgccga | |
| | gtacggctacatcagaacagtgctgggccagcagatcctg | |
| | ggacagctggattctagctctctggccctgccttctgagg | |
| | ccaagctgaaactggccggcagttctggaagaggcggcca | |
| | gacagtgaagtccctgcggatccaagaacaggtgcagcag | |
| | accctggccagaaagggcagatcttctgtcggcaacggca | |
| | acctgcacagaaccagctctgtgcccgagtacgtgtacaa | |
| | tctgcacctggtggaaaacgacttcgtcggcggcagatcc | |
| | cctgtgcctaagacctacgatatgctgaaggccggcacca | |
| | ccgccacctatgaaggcagatggggaagaggcacagccca | |
| | gtacagcagccagaaaagcgtggaagagagaagcctgcgg | |
| | caccctctgcggagactggaaatcagccctgatagcagcc | |
| | cagagagagcccactacacccacagcgactaccagtactc | |
| | ccagagatctcaggccggccacacactgcaccaccaagag | |
| | tctagaagggccgctctgctggtgcctcctagatacgcca | |
| | gatctgagatcgtgggcgtgtccagagccggcacaacaag | |
| | cagacagagacacttcgacacctaccaccggcagtatcag | |
| | cacggcagcgtgtccgataccgtgttcgatagcatccccg | |
| | ccaatcctgctctgctgacataccctagacctggcacctc | |
| | cagatccatgggcaatctgctggaaaaagagaactacctg | |
| | accgccggactgaccgtgggacaagttcgacctctggttc | |
| | ctctgcagcccgtgacacagaacagagccagcagaagcag | |
| | ctggcaccagtccagcttccacagcaccagaacactgaga | |
| | gaagctggccctagcgtggccgtggattcttctggtagaa | |
| | gggctcacctgacagttggccaagcagctgcaggcggaag | |
| | cggaaatctgctgaccgagagaagcaccttcaccgacagc | |
| | cagctgggcaacgccgacatggaaatgacactggaacggg | |
| | ccgtgtccatgctggaagccgatcacatgctgcccagcag | |
| | aattagcgccgctgccacctttatccagcacgagtgcttc | |
| | cagaagtctgaggcccggaagagagtgaaccagctgagag | |
| | gcatcctgaagctgctgcagctcctgaaggtgcagaacga | |
| | ggatgtgcagagggctgtgtgtggggccctgagaaatctg | |
| | gtgttcgaggacaacgacaacaagctggaagtggccgagc | |
| | tgaacggcgtgccaagactgctgcaggttctgaaacagac | |
| | ccgcgacctggaaacaaagaagcagatcaccggcctgctc | |
| | tggaacctgagcagcaacgacaagctgaagaacctgatga | |
| | tcacagaggccctgctgaccctgacagagaacatcatcat | |
| | ccctttcagcggctggcccgagggcgattaccctaaagct | |
| | aatggcctgctggacttcgacatcttctacaacgtgaccg | |
| | gctgcctgagaaacatgtctagcgctggcgccgatggcag | |
| | aaaggccatgagaagatgtgacggcctgatcgacagcctg | |
| | gtgcactatgtgcggggcacaatcgccgattaccagcctg | |
| | atgataaggccaccgagaactgcgtgtgcatcctgcacaa | |
| | cctgagctaccagctggaagcagagctgcccgagaagtac | |
| | agccagaacatctacatccagaaccggaacatccagaccg | |
| | acaacaacaagagcatcggctgcttcggcagccgcagccg | |
| | gaaagtgaaagaacagtaccaggacgtgcccatgcctgag | |
| | gaaaagtctaaccccaaaggcgtggaatggctgtggcaca | |
| | gcatcgtgatccggatgtacctgagcctgatcgccaagag | |
| | cgtgcggaattacacccaagaggcatctctgggcgccctg | |
| | cagaatctgacagcaggatctggccctatgcctacctctg | |
| | tggctcagaccgtggtgcagaaagagtctggcctgcagca | |
| | cacccggaagatgctgcatgtgggagatcccagcgtgaag | |
| | aaaaccgccatcagcctgctgagaaacctgagccggaatc | |
| | tgtctctgcagaatgagatcgccaaagagacactgcccga | |
| | cctggtgtctatcatccctgacaccgtgcctagcaccgac | |
| | ctgctgattgagacaacagccagcgcctgctacaccctga | |
| | acaacatcattcagaactcctaccagaacgcccgcgatct | |
| | gctgaacacaggcggcatccagaaaatcatggccatctct | |
| | gccggcgacgcctacgcctctaacaaggcctctaaagccg | |
| | ccagcgtgctgctgtattctctgtgggcccataccgagct | |
| | gcaccatgcctataagaaggcccagttcaaaaagaccgac | |
| | ttcgtgaacagccggaccgccaaggcctaccactctctga | |
| | aagattaataagcttggatccaatcaacctctggattaca | |
| | aaatttgtgaaagattgactggtattcttaactatgttgc | |
| | tccttttacgctatgtggatacgctgctttaatgcctttg | |
| | tatcatgctattgcttcccgtatggctttcattttctcct | |
| | ccttgtataaatcctggttgctgtctctttatgaggagtt | |
| | gtggcccgttgtcaggcaacgtggcgtggtgtgcactgtg | |
| | tttgctgacgcaacccccactggttggggcattgccacca | |
| | cctgtcagctcctttccgggactttcgctttccccctccc | |
| | tattgccacggcggaactcatcgccgcctgccttgcccgc | |
| | tgctggacaggggctcggctgttgggcactgacaattccg | |
| | tggtgttgtcggggaaatcatcgtcctttccttggctgct | |
| | cgcctgtgttgccacctggattctgcgcgggacgtccttc | |
| | tgctacgtcccttcggccctcaatccagcggaccttcctt | |
| | cccgcggcctgctgccggctctgcggcctcttccgcgtct | |
| | tcgagatctgcctcgactgtgccttctagttgccagccat | |
| | ctgttgtttgcccctcccccgtgccttccttgaccctgga | |
| | aggtgccactcccactgtcctttcctaataaaatgaggaa | |
| | attgcatcgcattgtctgagtaggtgtcattctattctgg | |
| | ggggtggggggggcaggacagcaagggggaggattgggaa | |
| | gacaatagcaggcatgctggggactggggactcgagttaa | |
| | gggcgaattcccgataaggatcttcctagagcatggctac | |
| | gtagataagtagcatggcgggttaatcattaactacaagg | |
| | aacccctagtgatggagttggccactccctctctgcgcgc | |
| | tcgctcgctcactgaggccgggcgaccaaaggtcgcccga | |
| | cgcccgggctttgcccgggggcctcagtgagcgagcgagc | |
| | gcgcagccttaattaacctaattcactggccgtcgtttta | |
| | caacgtcgtgactgggaaaaccctggcgttacccaactta | |
| | atcgccttgcagcacatccccctttcgccagctggcgtaa | |
| | tagcgaagaggcccgcaccgatcgcccttcccaacagttg | |
| | cgcagcctgaatggcgaatgggacgcgccctgtagcggcg | |
| | cattaagcgcggcgggtgtggtggttacgcgcagcgtgac | |
| | cgctacacttgccagcgccctagcgcccgctcctttcgct | |
| | ttcttcccttcctttctcgccacgttcgccggctttcccc | |
| | gtcaagctctaaatcgggggctccctttagggttccgatt | |
| | tagtgctttacggcacctcgaccccaaaaaacttgattag | |
| | ggtgatggttcacgtagtgggccatcgccccgatagacgg | |
| | tttttcgccctttgacgctggagttcacgttcctcaatag | |
| | tggactcttgttccaaactggaacaacactcaaccctatc | |
| | tcggtctattcttttgatttataagggatttttccgattt | |
| | cggcctattggttaaaaaatgagctgatttaacaaaaatt | |
| | taacgcgaattttaacaaaatattaacgtttataatttca | |
| | ggtggcatctttcggggaaatgtgcgcggaacccctattt | |
| | gtttatttttctaaatacattcaaatatgtatccgctcat | |
| | gagacaataaccctgataaatgcttcaataatattgaaaa | |
| | aggaagagtatgagtattcaacatttccgtgtcgccctta | |
| | ttcccttttttgcggcattttgccttcctgtttttgctca | |
| | cccagaaacgctggtgaaagtaaaagatgctgaagatcag | |
| | ttgggtgcacgagtgggttacatcgaactggatctcaata | |
| | gtggtaagatccttgagagttttcgccccgaagaacgttt | |
| | tccaatgatgagcacttttaaagttctgctatgtggcgcg | |
| | gtattatcccgtattgacgccgggcaagagcaactcggtc | |
| | gccgcatacactattctcagaatgacttggttgagtactc | |
| | accagtcacagaaaagcatcttacggatggcatgacagta | |
| | agagaattatgcagtgctgccataaccatgagtgataaca | |
| | ctgcggccaacttacttctgacaacgatcggaggaccgaa | |
| | ggagctaaccgcttttttgcacaacatgggggatcatgta | |
| | actcgccttgatcgttgggaaccggagctgaatgaagcca | |
| | taccaaacgacgagcgtgacaccacgatgcctgtagtaat | |
| | ggtaacaacgttgcgcaaactattaactggcgaactactt | |
| | actctagcttcccggcaacaattaatagactggatggagg | |
| | cggataaagttgcaggaccacttctgcgctcggcccttcc | |
| | ggctggctggtttattgctgataaatctggagccggtgag | |
| | cgtgggtctcgcggtatcattgcagcactggggccagatg | |
| | gtaagccctcccgtatcgtagttatctacacgacggggag | |
| | tcaggcaactatggatgaacgaaatagacagatcgctgag | |
| | ataggtgcctcactgattaagcattggtaactgtcagacc | |
| | aagtttactcatatatactttagattgatttaaaacttca | |
| | tttttaatttaaaaggatctaggtgaagatcctttttgat | |
| | aatctcatgaccaaaatcccttaacgtgagttttcgttcc | |
| | actgagcgtcagaccccgtagaaaagatcaaaggatcttc | |
| | ttgagatcctttttttctgcgcgtaatctgctgcttgcaa | |
| | acaaaaaaaccaccgctaccagcggtggtttgtttgccgg | |
| | atcaagagctaccaactctttttccgaaggtaactggctt | |
| | cagcagagcgcagataccaaatactgtccttctagtgtag | |
| | ccgtagttaggccaccacttcaagaactctgtagcaccgc | |
| | ctacatacctcgctctgctaatcctgttaccagtggctgc | |
| | tgccagtggcgataagtcgtgtcttaccgggttggactca | |
| | agacgatagttaccggataaggcgcagcggtcgggctgaa | |
| | cggggggttcgtgcacacagcccagcttggagcgaacgac | |
| | ctacaccgaactgagatacctacagcgtgagctatgagaa | |
| | agcgccacgcttcccgaagggagaaaggcggacaggtatc | |
| | cggtaagcggcagggtcggaacaggagagcgcacgaggga | |
| | gcttccagggggaaacgcctggtatctttatagtcctgtc | |
| | gggtttcgccacctctgacttgagcgtcgatttttgtgat | |
| | gctcgtcaggggggcggagcctatggaaaaacgccagcaa | |
| | cgcggcctttttacggttcctggccttttgctgcggtttt | |
| | gctcacatgttctttcctgcgttatcccctgattctgtgg | |
| | ataaccgtattaccgcctttgagtgagctgataccgctcg | |
| | ccgcagccgaacgaccgagcgcagcgagtcagtgagcgag | |
| | gaagcggaagagcgcccaatacgcaaaccgcctctccccg | |
| | cgcgttggccgattcattaatgcagctggcacgacaggtt | |
| | tcccgactggaaagcgggcagtgagcgcaacgcaattaat | |
| | gtgagttagctcactcattaggcaccccaggctttacact | |
| | ttatgcttccggctcgtatgttgtgtggaattgtgagcgg | |
| | ataacaatttcacacaggaaacagctatgaccatgattac | |
| | gccagatttaattaaggctgcgcgctcgctcgctcactga | |
| | ggccgcccgggcaaagcccgggcgtcgggcgacctttggt | |
| | cgcccggcctcagtgagcgagcgagcgcgcagagagggag | |
| | tggccaactccatcactaggggttccttgtagttaatgat | |
| | taacccgccatgctacttatctacgtagccatgctctagg | |
| | aagatcggaattc | |
| A helper | ggtacccaactccatgcttaacagtccccaggtacagccc | 2 |
| plasmid: | accctgcgtcgcaaccaggaacagctctacagcttcctgg | |
| pAdHelper | agcgccactcgccctacttccgcagccacagtgcgcagat | |
| HEK tfx T3 | taggagcgccacttctttttgtcacttgaaaaacatgtaa | |
| | aaataatgtactaggagacactttcaataaaggcaaatgt | |
| | ttttatttgtacactctcgggtgattatttaccccccacc | |
| | cttgccgtctgcgccgtttaaaaatcaaaggggttctgcc | |
| | gcgcatcgctatgcgccactggcagggacacgttgcgata | |
| | ctggtgtttagtgctccacttaaactcaggcacaaccatc | |
| | cgcggcagctcggtgaagttttcactccacaggctgcgca | |
| | ccatcaccaacgcgtttagcaggtcgggcgccgatatctt | |
| | gaagtcgcagttggggcctccgccctgcgcgcgcgagttg | |
| | cgatacacagggttgcagcactggaacactatcagcgccg | |
| | ggtggtgcacgctggccagcacgctcttgtcggagatcag | |
| | atccgcgtccaggtcctccgcgttgctcagggcgaacgga | |
| | gtcaactttggtagctgccttcccaaaaagggtgcatgcc | |
| | caggctttgagttgcactcgcaccgtagtggcatcagaag | |
| | gtgaccgtgcccggtctgggcgttaggatacagcgcctgc | |
| | atgaaagccttgatctgcttaaaagccacctgagcctttg | |
| | cgccttcagagaagaacatgccgcaagacttgccggaaaa | |
| | ctgattggccggacaggccgcgtcatgcacgcagcacctt | |
| | gcgtcggtgttggagatctgcaccacatttcggccccacc | |
| | ggttcttcacgatcttggccttgctagactgctccttcag | |
| | cgcgcgctgcccgttttcgctcgtcacatccatttcaatc | |
| | acgtgctccttatttatcataatgctcccgtgtagacact | |
| | taagctcgccttcgatctcagcgcagcggtgcagccacaa | |
| | cgcgcagcccgtgggctcgtggtgcttgtaggttacctct | |
| | gcaaacgactgcaggtacgcctgcaggaatcgccccatca | |
| | tcgtcacaaaggtcttgttgctggtgaaggtcagctgcaa | |
| | cccgcggtgctcctcgtttagccaggtcttgcatacggcc | |
| | gccagagcttccacttggtcaggcagtagcttgaagtttg | |
| | cctttagatcgttatccacgtggtacttgtccatcaacgc | |
| | gcgcgcagcctccatgcccttctcccacgcagacacgatc | |
| | ggcaggctcagcgggtttatcaccgtgctttcactttccg | |
| | cttcactggactcttccttttcctcttgcgtccgcatacc | |
| | ccgcgccactgggtcgtcttcattcagccgccgcaccgtg | |
| | cgcttacctcccttgccgtgcttgattagcaccggtgggt | |
| | tgctgaaacccaccatttgtagcgccacatcttctctttc | |
| | ttcctcgctgtccacgatcacctctggggatggcgggcgc | |
| | tcgggcttgggagaggggcgcttctttttctttttggacg | |
| | caatggccaaatccgccgtcgaggtcgatggccgcgggct | |
| | gggtgtgcgcggcaccagcgcatcttgtgacgagtcttct | |
| | tcgtcctcggactcgagacgccgcctcagccgcttttttg | |
| | ggggcgcgcggggaggcggcggcgacggcgacggggacga | |
| | cacgtcctccatggttggtggacgtcgcgccgcaccgcgt | |
| | ccgcgctcgggggtggtttcgcgctgctcctcttcccgac | |
| | tggccatttccttctcctataggcagaaaaagatcatgga | |
| | gtcagtcgagaaggaggacagcctaaccgccccctttgag | |
| | ttcgccaccaccgcctccaccgatgccgccaacgcgccta | |
| | ccaccttccccgtcgaggcacccccgcttgaggaggagga | |
| | agtgattatcgagcaggacccaggttttgtaagcgaagac | |
| | gacgaggatcgctcagtaccaacagaggataaaaagcaag | |
| | accaggacgacgcagaggcaaacgaggaacaagtcgggcg | |
| | gggggaccaaaggcatggcgactacctagatgtgggagac | |
| | gacgtgctgttgaagcatctgcagcgccagtgcgccatta | |
| | tctgcgacgcgttgcaagagcgcagcgatgtgcccctcgc | |
| | catagcggatgtcagccttgcctacgaacgccacctgttc | |
| | tcaccgcgcgtaccccccaaacgccaagaaaacggcacat | |
| | gcgagcccaacccgcgcctcaacttctaccccgtatttgc | |
| | cgtgccagaggtgcttgccacctatcacatctttttccaa | |
| | aactgcaagatacccctatcctgccgtgccaaccgcagcc | |
| | gagcggacaagcagctggccttgcggcagggcgctgtcat | |
| | acctgatatcgcctcgctcgacgaagtgccaaaaatcttt | |
| | gagggtcttggacgcgacgagaaacgcgcggcaaacgctc | |
| | tgcaacaagaaaacagcgaaaatgaaagtcactgtggagt | |
| | gctggtggaacttgagggtgacaacgcgcgcctagccgtg | |
| | ctgaaacgcagcatcgaggtcacccactttgcctacccgg | |
| | cacttaacctaccccccaaggttatgagcacagtcatgag | |
| | cgagctgatcgtgcgccgtgcacgacccctggagagggat | |
| | gcaaacttgcaagaacaaaccgaggagggcctacccgcag | |
| | ttggcgatgagcagctggcgcgctggcttgagacgcgcga | |
| | gcctgccgacttggaggagcgacgcaagctaatgatggcc | |
| | gcagtgcttgttaccgtggagcttgagtgcatgcagcggt | |
| | tctttgctgacccggagatgcagcgcaagctagaggaaac | |
| | gttgcactacacctttcgccagggctacgtgcgccaggcc | |
| | tgcaaaatttccaacgtggagctctgcaacctggtctcct | |
| | accttggaattttgcacgaaaaccgcctcgggcaaaacgt | |
| | gcttcattccacgctcaagggcgaggcgcgccgcgactac | |
| | gtccgcgactgcgtttacttatttctgtgctacacctggc | |
| | aaacggccatgggcgtgtggcagcaatgcctggaggagcg | |
| | caacctaaaggagctgcagaagctgctaaagcaaaacttg | |
| | aaggacctatggacggccttcaacgagcgctccgtggccg | |
| | cgcacctggcggacattatcttccccgaacgcctgcttaa | |
| | aaccctgcaacagggtctgccagacttcaccagtcaaagc | |
| | atgttgcaaaactttaggaactttatcctagagcgttcag | |
| | gaattctgcccgccacctgctgtgcgcttcctagcgactt | |
| | tgtgcccattaagtaccgtgaatgccctccgccgctttgg | |
| | ggtcactgctaccttctgcagctagccaactaccttgcct | |
| | accactccgacatcatggaagacgtgagcggtgacggcct | |
| | actggagtgtcactgtcgctgcaacctatgcaccccgcac | |
| | cgctccctggtctgcaattcgcaactgcttagcgaaagtc | |
| | aaattatcggtacctttgagctgcagggtccctcgcctga | |
| | cgaaaagtccgcggctccggggttgaaactcactccgggg | |
| | ctgtggacgtcggcttaccttcgcaaatttgtacctgagg | |
| | actaccacgcccacgagattaggttctacgaagaccaatc | |
| | ccgcccgccaaatgcggagcttaccgcctgcgtcattacc | |
| | cagggccacatccttggccaattgcaagccatcaacaaag | |
| | cccgccaagagtttctgctacgaaagggacggggggttta | |
| | cctggacccccagtccggcgaggagctcaacccaatcccc | |
| | ccgccgccgcagccctatcagcagccgcgggcccttgctt | |
| | cccaggatggcacccaaaaagaagctgcagctgccgccgc | |
| | cgccacccacggacgaggaggaatactgggacagtcaggc | |
| | agaggaggttttggacgaggaggaggagatgatggaagac | |
| | tgggacagcctagacgaagcttccgaggccgaagaggtgt | |
| | cagacgaaacaccgtcaccctcggtcgcattcccctcgcc | |
| | ggcgccccagaaattggcaaccgttcccagcatcgctaca | |
| | acctccgctcctcaggcgccgccggcactgcctgttcgcc | |
| | gacccaaccgtagatgggacaccactggaaccagggccgg | |
| | taagtctaagcagccgccgccgttagcccaagagcaacaa | |
| | cagcgccaaggctaccgctcgtggcgcgggcacaagaacg | |
| | ccatagttgcttgcttgcaagactgtgggggcaacatctc | |
| | cttcgcccgccgctttcttctctaccatcacggcgtggcc | |
| | ttcccccgtaacatcctgcattactaccgtcatctctaca | |
| | gcccctactgcaccggcggcagcggcagcggcagcaacag | |
| | cagcggtcacacagaagcaaaggcgaccggatagcaagac | |
| | tctgacaaagcccaagaaatccacagcggcggcagcagca | |
| | ggaggaggagcgctgcgtctggcgcccaacgaacccgtat | |
| | cgacccgcgagcttagaaataggatttttcccactctgta | |
| | tgctatatttcaacaaagcaggggccaagaacaagagctg | |
| | aaaataaaaaacaggtctctgcgctccctcacccgcagct | |
| | gcctgtatcacaaaagcgaagatcagcttcggcgcacgct | |
| | ggaagacgcggaggctctcttcagcaaatactgcgcgctg | |
| | actcttaaggactagtttcgcgccctttctcaaatttaag | |
| | cgcgaaaactacgtcatctccagcggccacacccggcgcc | |
| | agcacctgtcgtcagcgccattatgagcaaggaaattccc | |
| | acgccctacatgtggagttaccagccacaaatgggacttg | |
| | cggctggagctgcccaagactactcaacccgaataaacta | |
| | catgagcgcgggaccccacatgatatcccgggtcaacgga | |
| | atccgcgcccaccgaaaccgaattctcctcgaacaggcgg | |
| | ctattaccaccacacctcgtaataaccttaatccccgtag | |
| | ttggcccgctgccctggtgtaccaggaaagtcccgctccc | |
| | accactgtggtacttcccagagacgcccaggccgaagttc | |
| | agatgactaactcaggggcgcagcttgcgggcggctttcg | |
| | tcacagggtgcggtcgcccgggcgttttagggcggagtaa | |
| | cttgcatgtattgggaattgtagtttttttaaaatgggaa | |
| | gtgacgtatcgtgggaaaacggaagtgaagatttgaggaa | |
| | gttgtgggttttttggctttcgtttctgggcgtaggttcg | |
| | cgtgcggttttctgggtgttttttgtggactttaaccgtt | |
| | acgtcattttttagtcctatatatactcgctctgtacttg | |
| | gccctttttacactgtgactgattgagctggtgccgtgtc | |
| | gagtggtgttttttaataggtttttttactggtaaggctg | |
| | actgttatggctgccgctgtggaagcgctgtatgttgttc | |
| | tggagcgggagggtgctattttgcctaggcaggagggttt | |
| | ttcaggtgtttatgtgtttttctctcctattaattttgtt | |
| | atacctcctatgggggctgtaatgttgtctctacgcctgc | |
| | gggtatgtattcccccgggctatttcggtcgctttttagc | |
| | actgaccgatgttaaccaacctgatgtgtttaccgagtct | |
| | tacattatgactccggacatgaccgaggaactgtcggtgg | |
| | tgctttttaatcacggtgaccagtttttttacggtcacgc | |
| | cggcatggccgtagtccgtcttatgcttataagggttgtt | |
| | tttcctgttgtaagacaggcttctaatgtttaaatgtttt | |
| | tttttttgttattttattttgtgtttaatgcaggaacccg | |
| | cagacatgtttgagagaaaaatggtgtctttttctgtggt | |
| | ggttccggaacttacctgcctttatctgcatgagcatgac | |
| | tacgatgtgcttgcttttttgcgcgaggctttgcctgatt | |
| | ttttgagcagcaccttgcattttatatcgccgcccatgca | |
| | acaagcttacataggggctacgctggttagcatagctccg | |
| | agtatgcgtgtcataatcagtgtgggttcttttgtcatgg | |
| | ttcctggggggaagtggccgcgctggtccgtgcagacctg | |
| | cacgattatgttcagctggccctgcgaagggacctacggg | |
| | atcgcggtatttttgttaatgttccgcttttgaatcttat | |
| | acaggtctgtgaggaacctgaatttttgcaatcatgattc | |
| | gctgcttgaggctgaaggggagggcgctctggagcagatt | |
| | tttacaatggccggacttaatattcgggatttgcttagag | |
| | acatattgataaggtggcgagatgaaaattatttgggcat | |
| | ggttgaaggtgctggaatgtttatagaggagattcaccct | |
| | gaagggtttagcctttacgtccacttggacgtgagggcag | |
| | tttgccttttggaagccattgtgcaacatcttacaaatgc | |
| | cattatctgttctttggctgtagagtttgaccacgccacc | |
| | ggaggggagcgcgttcacttaatagatcttcattttgagg | |
| | ttttggataatcttttggaataaaaaaaaaaaaacatggt | |
| | tcttccagctcttcccgctcctcccgtgtgtgactcgcag | |
| | aacgaatgtgtaggttggctgggtgtggcttattctgcgg | |
| | tggtggatgttatcagggcagcggcgcatgaaggagttta | |
| | catagaacccgaagccagggggcgcctggatgctttgaga | |
| | gagtggatatactacaactactacacagagcgagctaagc | |
| | gacgagaccggagacgcagatctgtttgtcacgcccgcac | |
| | ctggttttgcttcaggaaatatgactacgtccggcgttcc | |
| | atttggcatgacactacgaccaacacgatctcggttgtct | |
| | cggcgcactccgtacagtagggatcgcctacctccttttg | |
| | agacagagacccgcgctaccatactggaggatcatccgct | |
| | gctgcccgaatgtaacactttgacaatgcacaacgtgagt | |
| | tacgtgcgaggtcttccctgcagtgtgggatttacgctga | |
| | ttcaggaatgggttgttccctgggatatggttctgacgcg | |
| | ggaggagcttgtaatcctgaggaagtgtatgcacgtgtgc | |
| | ctgtgttgtgccaacattgatatcatgacgagcatgatga | |
| | tccatggttacgagtcctgggctctccactgtcattgttc | |
| | cagtcccggttccctgcagtgcatagccggcgggcaggtt | |
| | ttggccagctggtttaggatggtggtggatggcgccatgt | |
| | ttaatcagaggtttatatggtaccgggaggtggtgaatta | |
| | caacatgccaaaagaggtaatgtttatgtccagcgtgttt | |
| | atgaggggtcgccacttaatctacctgcgcttgtggtatg | |
| | atggccacgtgggttctgtggtccccgccatgagctttgg | |
| | atacagcgccttgcactgtgggattttgaacaatattgtg | |
| | gtgctgtgctgcagttactgtgctgatttaagtgagatca | |
| | gggtgcgctgctgtgcccggaggacaaggcgtctcatgct | |
| | gcgggcggtgcgaatcatcgctgaggagaccactgccatg | |
| | ttgtattcctgcaggacggagcggcggcggcagcagttta | |
| | ttcgcgcgctgctgcagcaccaccgccctatcctgatgca | |
| | cgattatgactctacccccatgtaggcgtggacttcccct | |
| | tcgccgcccgttgagcaaccgcaagttggacagcagcctg | |
| | tggctcagcagctggacagcgacatgaacttaagcgagct | |
| | gcccggggagtttattaatatcactgatgagcgtttggct | |
| | cgacaggaaaccgtgtggaatataacacctaagaatatgt | |
| | ctgttacccatgatatgatgctttttaaggccagccgggg | |
| | agaaaggactgtgtactctgtgtgttgggagggaggtggc | |
| | aggttgaatactagggttctgtgagtttgattaaggtacg | |
| | gtgatcaatataagctatgtggtggtggggctatactact | |
| | gaatgaaaaatgacttgaaattttctgcaattgaaaaata | |
| | aacacgttgaaacataacatgcaacaggttcacgattctt | |
| | tattcctgggcaatgtaggagaaggtgtaagagttggtag | |
| | caaaagtttcagtggtgtattttccactttcccaggacca | |
| | tgtaaaagacatagagtaagtgcttacctcgctagtttct | |
| | gtggattcactagaatcgatgtaggatgttgcccctcctg | |
| | acgcggtaggagaaggggagggtgccctgcatgtctgccg | |
| | ctgctcttgctcttgccgctgctgaggaggggggcgcatc | |
| | tgccgcagcaccggatgcatctgggaaaagcaaaaaaggg | |
| | gctcgtccctgtttccggaggaatttgcaagcggggtctt | |
| | gcatgacggggaggcaaacccccgttcgccgcagtccggc | |
| | cggcccgagactcgaaccgggggtcctgcgactcaaccct | |
| | tggaaaataaccctccggctacagggagcgagccacttaa | |
| | tgctttcgctttccagcctaaccgcttacgccgcgcgcgg | |
| | ccagtggccaaaaaagctagcgcagcagccgccgcgcctg | |
| | gaaggaagccaaaaggagcgctcccccgttgtctgacgtc | |
| | gcacacctgggttcgacacgcgggcggtaaccgcatggat | |
| | cacggcggacggccggatccggggttcgaaccccggtcgt | |
| | ccgccatgatacccttgcgaatttatccaccagaccacgg | |
| | aagagtgcccgcttacaggctctccttttgcacggtctag | |
| | agcgtcaacgactgcgcacgcctcaccggccagagcgtcc | |
| | cgaccatggagcactttttgccgctgcgcaacatctggaa | |
| | ccgcgtccgcgactttccgcgcgcctccaccaccgccgcc | |
| | ggcatcacctggatgtccaggtacatctacggattacgtc | |
| | gacgtttaaaccatatgatcagctcactcaaaggcggtaa | |
| | tacggttatccacagaatcaggggataacgcaggaaagaa | |
| | catgtgagcaaaaggccagcaaaaggccaggaaccgtaaa | |
| | aaggccgcgttgctggcgtttttccataggctccgccccc | |
| | ctgacgagcatcacaaaaatcgacgctcaagtcagaggtg | |
| | gcgaaacccgacaggactataaagataccaggcgtttccc | |
| | cctggaagctccctcgtgcgctctcctgttccgaccctgc | |
| | cgcttaccggatacctgtccgcctttctcccttcgggaag | |
| | cgtggcgctttctcatagctcacgctgtaggtatctcagt | |
| | tcggtgtaggtcgttcgctccaagctgggctgtgtgcacg | |
| | aaccccccgttcagcccgaccgctgcgccttatccggtaa | |
| | ctatcgtcttgagtccaacccggtaagacacgacttatcg | |
| | ccactggcagcagccactggtaacaggattagcagagcga | |
| | ggtatgtaggcggtgctacagagttcttgaagtggtggcc | |
| | taactacggctacactagaagaacagtatttggtatctgc | |
| | gctctgctgaagccagttaccttcggaaaaagagttggta | |
| | gctcttgatccggcaaacaaaccaccgctggtagcggtgg | |
| | tttttttgtttgcaagcagcagattacgcgcagaaaaaaa | |
| | ggatctcaagaagatcctttgatcttttctacggggtctg | |
| | acgctcagtggaacgaaaactcacgttaagggattttggt | |
| | catgagattatcaaaaaggatcttcacctagatcctttta | |
| | aattaaaaatgaagttttaaatcaatctaaagtatatatg | |
| | agtaaacttggtctgacagttaccaatgcttaatcagtga | |
| | ggcacctatctcagcgatctgtctatttcgttcatccata | |
| | gttgcctgactccccgtcgtgtagataactacgatacggg | |
| | agggcttaccatctggccccagtgctgcaatgataccgcg | |
| | agacccacgctcaccggctccagatttatcagcaataaac | |
| | cagccagccggaagggccgagcgcagaagtggtcctgcaa | |
| | ctttatccgcctccatccagtctattaattgttgccggga | |
| | agctagagtaagtagttcgccagttaatagtttgcgcaac | |
| | gttgttgccattgctacaggcatcgtggtgtcacgctcgt | |
| | cgtttggtatggcttcattcagctccggttcccaacgatc | |
| | aaggcgagttacatgatcccccatgttgtgcaaaaaagcg | |
| | gttagctccttcggtcctccgatcgttgtcagaagtaagt | |
| | tggccgcagtgttatcactcatggttatggcagcactgca | |
| | taattctcttactgtcatgccatccgtaagatgcttttct | |
| | gtgactggtgagtactcaaccaagtcattctgagaatagt | |
| | gtatgcggcgaccgagttgctcttgcccggcgtcaatacg | |
| | ggataataccgcgccacatagcagaactttaaaagtgctc | |
| | atcattggaaaacgttcttcggggcgaaaactctcaagga | |
| | tcttaccgctgttgagatccagttcgatgtaacccactcg | |
| | tgcacccaactgatcttcagcatcttttactttcaccagc | |
| | gtttctgggtgagcaaaaacaggaaggcaaaatgccgcaa | |
| | aaaagggaataagggcgacacggaaatgttgaatactcat | |
| | actcttcctttttcaatattattgaagcatttatcagggt | |
| | tattgtctcatgagcggatacatatttgaatgtatttaga | |
| | aaaataaacaaataggggttccgcgcacatttccccgaaa | |
| | agtgccacctaaattgtaagcgttaatattttgttaaaat | |
| | tcgcgttaaatttttgttaaatcagctcattttttaacca | |
| | ataggccgaaatcggcaaaatcccttataaatcaaaagaa | |
| | tagaccgagatagggttgagtgttgttccagtttggaaca | |
| | agagtccactattaaagaacgtggactccaacgtcaaagg | |
| | gcgaaaaaccgtctatcagggcgatggcccactacgtgaa | |
| | ccatcaccctaatcaagttttttggggtcgaggtgccgta | |
| | aagcactaaatcggaaccctaaagggagcccccgatttag | |
| | agcttgacggggaaagccggcgaacgtggcgagaaaggaa | |
| | gggaagaaagcgaaaggagcgggcgctagggcgctggcaa | |
| | gtgtagcggtcacgctgcgcgtaaccaccacacccgccgc | |
| | gcttaatgcgccgctacagggcgcgatggatcc | |
| A rep/cap | tcgcgcgtttcggtgatgacggtgaaaacctctgacacat | 3 |
| plasmid with | gcagctcccggagacggtcacagcttgtctgtaagcggat | |
| wild-type | gccgggagcagacaagcccgtcagggcgcgtcagcgggtg | |
| AAV9 cap: | ttggcgggtgtcggggctggcttaactatgcggcatcaga | |
| pZC100_REP2 | gcagattgtactgagagtgcaccatatgcggtgtgaaata | |
| AAV9 | ccgcacagatgcgtaaggagaaaataccgcatcaggcgcc | |
| | attcgccattcaggctgcgcaactgttgggaagggcgatc | |
| | ggtgcgggcctcttcgctattacgccagctggcgaaaggg | |
| | ggatgtgctgcaaggcgattaagttgggtaacgccagggt | |
| | tttcccagtcacgacgttgtaaaacgacggccagtgaatt | |
| | gacgcgtattgggatgtagttaatgattaacccgccatgc | |
| | tacttatctacgtagccatgctctaggaagatcggaattc | |
| | gccctattagagtcctgtattagaggtcacgtgagtgttt | |
| | tgcgacattttgcgacaccatgtggtcacgctgggtattt | |
| | aagcccgagtgagcacgcagggtctccattttgaagcggg | |
| | aggtttgaacgcgcagccaccacggcggggttttacgaga | |
| | ttgtgattaaggtccccagcgaccttgacgagcatctgcc | |
| | cggcatttctgacagctttgtgaactgggtggccgagaag | |
| | gaatgggagttgccgccagattctgacatggatctgaatc | |
| | tgattgagcaggcacccctgaccgtggccgagaagctgca | |
| | gcgcgactttctgacggaatggcgccgtgtgagtaaggcc | |
| | ccggaggcccttttctttgtgcaatttgagaagggagaga | |
| | gctacttccacatgcacgtgctcgtggaaaccaccggggt | |
| | gaaatccatggttttgggacgtttcctgagtcagattcgc | |
| | gaaaaactgattcagagaatttaccgcgggatcgagccga | |
| | ctttgccaaactggttcgcggtcacaaagaccagaaatgg | |
| | cgccggaggcgggaacaaggtggtggatgagtgctacatc | |
| | cccaattacttgctccccaaaacccagcctgagctccagt | |
| | gggcgtggactaatatggaacagtatttaagcgcctgttt | |
| | gaatctcacggagcgtaaacggttggtggcgcagcatctg | |
| | acgcacgtgtcgcagacgcaggagcagaacaaagagaatc | |
| | agaatcccaattctgatgcgccggtgatcagatcaaaaac | |
| | ttcagccaggtacatggagctggtcgggtggctcgtggac | |
| | aaggggattacctcggagaagcagtggatccaggaggacc | |
| | aggcctcatacatctccttcaatgcggcctccaactcgcg | |
| | gtcccaaatcaaggctgccttggacaatgcgggaaagatt | |
| | atgagcctgactaaaaccgcccccgactacctggtgggcc | |
| | agcagcccgtggaggacatttccagcaatcggatttataa | |
| | aattttggaactaaacgggtacgatccccaatatgcggct | |
| | tccgtctttctgggatgggccacgaaaaagttcggcaaga | |
| | ggaacaccatctggctgtttgggcctgcaactaccgggaa | |
| | gaccaacatcgcggaggccatagcccacactgtgcccttc | |
| | tacgggtgcgtaaactggaccaatgagaactttcccttca | |
| | acgactgtgtcgacaagatggtgatctggtgggaggaggg | |
| | gaagatgaccgccaaggtcgtggagtcggccaaagccatt | |
| | ctcggaggaagcaaggtgcgcgtggaccagaaatgcaagt | |
| | cctcggcccagatagacccgactcccgtgatcgtcacctc | |
| | caacaccaacatgtgcgccgtgattgacgggaactcaacg | |
| | accttcgaacaccagcagccgttgcaagaccggatgttca | |
| | aatttgaactcacccgccgtctggatcatgactttgggaa | |
| | ggtcaccaagcaggaagtcaaagactttttccggtgggca | |
| | aaggatcacgtggttgaggtggagcatgaattctacgtca | |
| | aaaagggtggagccaagaaaagacccgcccccagtgacgc | |
| | agatataagtgagcccaaacgggtgcgcgagtcagttgcg | |
| | cagccatcgacgtcagacgcggaagcttcgatcaactacg | |
| | cagacaggtaccaaaacaaatgttctcgtcacgtgggcat | |
| | gaatctgatgctgtttccctgcagacaatgcgagagaatg | |
| | aatcagaattcaaatatctgcttcactcacggacagaaag | |
| | actgtttagagtgctttcccgtgtcagaatctcaacccgt | |
| | ttctgtcgtcaaaaaggcgtatcagaaactgtgctacatt | |
| | catcatatcatgggaaaggtgccagacgcttgcactgcct | |
| | gcgatctggtcaatgtggatttggatgactgcatctttga | |
| | acaataaatgacttaaaccaggtatggctgccgatggtta | |
| | tcttccagattggctcgaggacaaccttagtgaaggaatt | |
| | cgcgagtggtgggctttgaaacctggagcccctcaaccca | |
| | aggcaaatcaacaacatcaagacaacgctcgaggtcttgt | |
| | gcttccgggttacaaataccttggacccggcaacggactc | |
| | gacaagggggagccggtcaacgcagcagacgcggcggccc | |
| | tcgagcacgacaaggcctacgaccagcagctcaaggccgg | |
| | agacaacccgtacctcaagtacaaccacgccgacgccgag | |
| | ttccaggagcggctcaaagaagatacgtcttttgggggca | |
| | acctcgggcgagcagtcttccaggccaaaaagaggcttct | |
| | tgaacctcttggtctggttgaggaagcggctaagacggct | |
| | cctggaaagaagaggcctgtagagcagtctcctcaggaac | |
| | cggactcctccgcgggtattggcaaatcgggtgcacagcc | |
| | cgctaaaaagagactcaatttcggtcagactggcgacaca | |
| | gagtcagtcccagaccctcaaccaatcggagaacctcccg | |
| | cagccccctcaggtgtgggatctcttacaatggcttcagg | |
| | tggtggcgcaccagtggcagacaataacgaaggtgccgat | |
| | ggagtgggtagttcctcgggaaattggcattgcgattccc | |
| | aatggctgggggacagagtcatcaccaccagcacccgaac | |
| | ctgggccctgcccacctacaacaatcacctctacaagcaa | |
| | atctccaacagcacatctggaggatcttcaaatgacaacg | |
| | cctacttcggctacagcaccccctgggggtattttgactt | |
| | caacagattccactgccacttctcaccacgtgactggcag | |
| | cgactcatcaacaacaactggggattccggcctaagcgac | |
| | tcaacttcaagctcttcaacattcaggtcaaagaggttac | |
| | ggacaacaatggagtcaagaccatcgccaataaccttacc | |
| | agcacggtccaggtcttcacggactcagactatcagctcc | |
| | cgtacgtgctcgggtcggctcacgagggctgcctcccgcc | |
| | gttcccagcggacgttttcatgattcctcagtacgggtat | |
| | ctgacgcttaatgatggaagccaggccgtgggtcgttcgt | |
| | ccttttactgcctggaatatttcccgtcgcaaatgctaag | |
| | aacgggtaacaacttccagttcagctacgagtttgagaac | |
| | gtacctttccatagcagctacgctcacagccaaagcctgg | |
| | accgactaatgaatccactcatcgaccaatacttgtacta | |
| | tctctcaaagactattaacggttctggacagaatcaacaa | |
| | acgctaaaattcagtgtggccggacccagcaacatggctg | |
| | tccagggaagaaactacatacctggacccagctaccgaca | |
| | acaacgtgtctcaaccactgtgactcaaaacaacaacagc | |
| | gaatttgcttggcctggagcttcttcttgggctctcaatg | |
| | gacgtaatagcttgatgaatcctggacctgctatggccag | |
| | ccacaaagaaggagaggaccgtttctttcctttgtctgga | |
| | tctttaatttttggcaaacaaggaactggaagagacaacg | |
| | tggatgcggacaaagtcatgataaccaacgaagaagaaat | |
| | taaaactactaacccggtagcaacggagtcctatggacaa | |
| | gtggccacaaaccaccagagtgcccaagcacaggcgcaga | |
| | ccggctgggttcaaaaccaaggaatacttccgggtatggt | |
| | ttggcaggacagagatgtgtacctgcaaggacccatttgg | |
| | gccaaaattcctcacacggacggcaactttcacccttctc | |
| | cgctgatgggagggtttggaatgaagcacccgcctcctca | |
| | gatcctcatcaaaaacacacctgtacctgcggatcctcca | |
| | acggccttcaacaaggacaagctgaactctttcatcaccc | |
| | agtattctactggccaagtcagcgtggagatcgagtggga | |
| | gctgcagaaggaaaacagcaagcgctggaacccggagatc | |
| | cagtacacttccaactattacaagtctaataatgttgaat | |
| | ttgctgttaatactgaaggtgtatatagtgaaccccgccc | |
| | cattggcaccagatacctgactcgtaatctgtaattgctt | |
| | gttaatcaataaaccgtttaattcgtttcagttgaacttt | |
| | ggtctctgcgaaggcgcgccataacttcgttggcgtaatc | |
| | atggtcatagctgtttcctgtgtgaaattgttatccgctc | |
| | acaattccacacaacatacgagccggaagcataaagtgta | |
| | aagcctggggtgcctaatgagtgagctaactcacattaat | |
| | tgcgttgcgctcactgcccgctttccagtcgggaaacctg | |
| | tcgtgccagctgcattaatgaatcggccaacgcgcgggga | |
| | gaggcggtttgcgtattgggcgctcttccgcttcctcgct | |
| | cactgactcgctgcgctcggtcgttcggctgcggcgagcg | |
| | gtatcagctcactcaaaggcggtaatacggttatccacag | |
| | aatcaggggataacgcaggaaagaacatgtgagcaaaagg | |
| | ccagcaaaaggccaggaaccgtaaaaaggccgcgttgctg | |
| | gcgtttttccataggctccgcccccctgacgagcatcaca | |
| | aaaatcgacgctcaagtcagaggtggcgaaacccgacagg | |
| | actataaagataccaggcgtttccccctggaagctccctc | |
| | gtgcgctctcctgttccgaccctgccgcttaccggatacc | |
| | tgtccgcctttctcccttcgggaagcgtggcgctttctca | |
| | tagctcacgctgtaggtatctcagttcggtgtaggtcgtt | |
| | cgctccaagctgggctgtgtgcacgaaccccccgttcagc | |
| | ccgaccgctgcgccttatccggtaactatcgtcttgagtc | |
| | caacccggtaagacacgacttatcgccactggcagcagcc | |
| | actggtaacaggattagcagagcgaggtatgtaggcggtg | |
| | ctacagagttcttgaagtggtggcctaactacggctacac | |
| | tagaagaacagtatttggtatctgcgctctgctgaagcca | |
| | gttaccttcggaaaaagagttggtagctcttgatccggca | |
| | aacaaaccaccgctggtagcggtggtttttttgtttgcaa | |
| | gcagcagattacgcgcagaaaaaaaggatctcaagaagat | |
| | cctttgatcttttctacggggtctgacgctcagtggaacg | |
| | aaaactcacgttaagggattttggtcatgagattatcaaa | |
| | aaggatcttcacctagatccttttaaattaaaaatgaagt | |
| | tttaaatcaatctaaagtatatatgagtaaacttggtctg | |
| | acagttaccaatgcttaatcagtgaggcacctatctcagc | |
| | gatctgtctatttcgttcatccatagttgcctgactcccc | |
| | gtcgtgtagataactacgatacgggagggcttaccatctg | |
| | gccccagtgctgcaatgataccgcgagacccacgctcacc | |
| | ggctccagatttatcagcaataaaccagccagccggaagg | |
| | gccgagcgcagaagtggtcctgcaactttatccgcctcca | |
| | tccagtctattaattgttgccgggaagctagagtaagtag | |
| | ttcgccagttaatagtttgcgcaacgttgttgccattgct | |
| | acaggcatcgtggtgtcacgctcgtcgtttggtatggctt | |
| | cattcagctccggttcccaacgatcaaggcgagttacatg | |
| | atcccccatgttgtgcaaaaaagcggttagctccttcggt | |
| | cctccgatcgttgtcagaagtaagttggccgcagtgttat | |
| | cactcatggttatggcagcactgcataattctcttactgt | |
| | catgccatccgtaagatgcttttctgtgactggtgagtac | |
| | tcaaccaagtcattctgagaatagtgtatgcggcgaccga | |
| | gttgctcttgcccggcgtcaatacgggataataccgcgcc | |
| | acatagcagaactttaaaagtgctcatcattggaaaacgt | |
| | tcttcggggcgaaaactctcaaggatcttaccgctgttga | |
| | gatccagttcgatgtaacccactcgtgcacccaactgatc | |
| | ttcagcatcttttactttcaccagcgtttctgggtgagca | |
| | aaaacaggaaggcaaaatgccgcaaaaaagggaataaggg | |
| | cgacacggaaatgttgaatactcatactcttcctttttca | |
| | atattattgaagcatttatcagggttattgtctcatgagc | |
| | ggatacatatttgaatgtatttagaaaaataaacaaatag | |
| | gggttccgcgcacatttccccgaaaagtgccacctgacgt | |
| | ctaagaaaccattattatcatgacattaacctataaaaat | |
| | aggcgtatcacgaggccctttcgtc | |
| A rep/cap | tcgcgcgtttcggtgatgacggtgaaaacctctgacacat | 4 |
| plasmid with | gcagctcccggagacggtcacagcttgtctgtaagcggat | |
| wild-type | gccgggagcagacaagcccgtcagggcgcgtcagcgggtg | |
| AAV5 cap: | ttggcgggtgtcggggctggcttaactatgcggcatcaga | |
| pZC107_REP2- | gcagattgtactgagagtgcaccatatgcggtgtgaaata | |
| AAV5 | ccgcacagatgcgtaaggagaaaataccgcatcaggcgcc | |
| | attcgccattcaggctgcgcaactgttgggaagggcgatc | |
| | ggtgcgggcctcttcgctattacgccagctggcgaaaggg | |
| | ggatgtgctgcaaggcgattaagttgggtaacgccagggt | |
| | tttcccagtcacgacgttgtaaaacgacggccagtgaatt | |
| | gacgcgtattgggatgtagttaatgattaacccgccatgc | |
| | tacttatctacgtagccatgctctaggaagatcggaattc | |
| | gccctattagagtcctgtattagaggtcacgtgagtgttt | |
| | tgcgacattttgcgacaccatgtggtcacgctgggtattt | |
| | aagcccgagtgagcacgcagggtctccattttgaagcggg | |
| | aggtttgaacgcgcagccaccacggcggggttttacgaga | |
| | ttgtgattaaggtccccagcgaccttgacgagcatctgcc | |
| | cggcatttctgacagctttgtgaactgggtggccgagaag | |
| | gaatgggagttgccgccagattctgacatggatctgaatc | |
| | tgattgagcaggcacccctgaccgtggccgagaagctgca | |
| | gcgcgactttctgacggaatggcgccgtgtgagtaaggcc | |
| | ccggaggcccttttctttgtgcaatttgagaagggagaga | |
| | gctacttccacatgcacgtgctcgtggaaaccaccggggt | |
| | gaaatccatggttttgggacgtttcctgagtcagattcgc | |
| | gaaaaactgattcagagaatttaccgcgggatcgagccga | |
| | ctttgccaaactggttcgcggtcacaaagaccagaaatgg | |
| | cgccggaggcgggaacaaggtggtggatgagtgctacatc | |
| | cccaattacttgctccccaaaacccagcctgagctccagt | |
| | gggcgtggactaatatggaacagtatttaagcgcctgttt | |
| | gaatctcacggagcgtaaacggttggtggcgcagcatctg | |
| | acgcacgtgtcgcagacgcaggagcagaacaaagagaatc | |
| | agaatcccaattctgatgcgccggtgatcagatcaaaaac | |
| | ttcagccaggtacatggagctggtcgggtggctcgtggac | |
| | aaggggattacctcggagaagcagtggatccaggaggacc | |
| | aggcctcatacatctccttcaatgcggcctccaactcgcg | |
| | gtcccaaatcaaggctgccttggacaatgcgggaaagatt | |
| | atgagcctgactaaaaccgcccccgactacctggtgggcc | |
| | agcagcccgtggaggacatttccagcaatcggatttataa | |
| | aattttggaactaaacgggtacgatccccaatatgcggct | |
| | tccgtctttctgggatgggccacgaaaaagttcggcaaga | |
| | ggaacaccatctggctgtttgggcctgcaactaccgggaa | |
| | gaccaacatcgcggaggccatagcccacactgtgcccttc | |
| | tacgggtgcgtaaactggaccaatgagaactttcccttca | |
| | acgactgtgtcgacaagatggtgatctggtgggaggaggg | |
| | gaagatgaccgccaaggtcgtggagtcggccaaagccatt | |
| | ctcggaggaagcaaggtgcgcgtggaccagaaatgcaagt | |
| | cctcggcccagatagacccgactcccgtgatcgtcacctc | |
| | caacaccaacatgtgcgccgtgattgacgggaactcaacg | |
| | accttcgaacaccagcagccgttgcaagaccggatgttca | |
| | aatttgaactcacccgccgtctggatcatgactttgggaa | |
| | ggtcaccaagcaggaagtcaaagactttttccggtgggca | |
| | aaggatcacgtggttgaggtggagcatgaattctacgtca | |
| | aaaagggtggagccaagaaaagacccgcccccagtgacgc | |
| | agatataagtgagcccaaacgggtgcgcgagtcagttgcg | |
| | cagccatcgacgtcagacgcggaagcttcgatcaactacg | |
| | cagacaggtaccaaaacaaatgttctcgtcacgtgggcat | |
| | gaatctgatgctgtttccctgcagacaatgcgagagaatg | |
| | aatcagaattcaaatatctgcttcactcacggacagaaag | |
| | actgtttagagtgctttcccgtgtcagaatctcaacccgt | |
| | ttctgtcgtcaaaaaggcgtatcagaaactgtgctacatt | |
| | catcatatcatgggaaaggtgccagacgcttgcactgcct | |
| | gcgatctggtcaatgtggatttggatgactgcatctttga | |
| | acaataaatgacttaaaccaggtatgtcttttgttgatca | |
| | ccctccagattggttggaagaagttggtgaaggtcttcgc | |
| | gagtttttgggccttgaaggggcccaccgaaaccaaaacc | |
| | caatcagcagcatcaagatcaagcccgtggtcttgtgctg | |
| | cctggttataactatctcggacccggaaacggtctcgatc | |
| | gaggagagcctgtcaacagggcagacgaggtcgcgcgaga | |
| | gcacgacatctcgtacaacgagcagcttgaggcgggagac | |
| | aacccctacctcaagtacaaccacgcggacgccgagtttc | |
| | aggagaagctcgccgacgacacatccttcgggggaaacct | |
| | cggaaaggcagtctttcaggccaagaaaagggttctcgaa | |
| | ccttttggcctggttgaagagggtgctaagacggccccta | |
| | ccggaaagcggatagacgaccactttccaaaaagaaagaa | |
| | ggctcggaccgaagaggactccaagccttccacctcgtca | |
| | gacgccgaagctggacccagcggatcccagcagctgcaaa | |
| | tcccagcccaaccagcctcaagtttgggagctgatacaat | |
| | gtctgcgggaggtggcggcccattgggcgacaataaccaa | |
| | ggtgccgatggagtgggcaatgcctcgggagattggcatt | |
| | gcgattccacgtggatgggggacagagtcgtcaccaagtc | |
| | cacccgaacctgggtgctgcccagctacaacaaccaccag | |
| | taccgagagatcaaaagcggctccgtcgacggaagcaacg | |
| | ccaacgcctactttggatacagcaccccctgggggtactt | |
| | tgactttaaccgcttccacagccactggagcccccgagac | |
| | tggcaaagactcatcaacaactactggggcttcagacccc | |
| | ggtccctcagagtcaaaatcttcaacattcaagtcaaaga | |
| | ggtcacggtgcaggactccaccaccaccatcgccaacaac | |
| | ctcacctccaccgtccaagtgtttacggacgacgactacc | |
| | agctgccctacgtcgtcggcaacgggaccgagggatgcct | |
| | gccggccttccctccgcaggtctttacgctgccgcagtac | |
| | ggttacgcgacgctgaaccgcgacaacacagaaaatccca | |
| | ccgagaggagcagcttcttctgcctagagtactttcccag | |
| | caagatgctgagaacgggcaacaactttgagtttacctac | |
| | aactttgaggaggtgcccttccactccagcttcgctccca | |
| | gtcagaacctcttcaagctggccaacccgctggtggacca | |
| | gtacttgtaccgcttcgtgagcacaaataacactggcgga | |
| | gtccagttcaacaagaacctggccgggagatacgccaaca | |
| | cctacaaaaactggttcccggggcccatgggccgaaccca | |
| | gggctggaacctgggctccggggtcaaccgcgccagtgtc | |
| | agcgccttcgccacgaccaataggatggagctcgagggcg | |
| | cgagttaccaggtgcccccgcagccgaacggcatgaccaa | |
| | caacctccagggcagcaacacctatgccctggagaacact | |
| | atgatcttcaacagccagccggcgaacccgggcaccaccg | |
| | ccacgtacctcgagggcaacatgctcatcaccagcgagag | |
| | cgagacgcagccggtgaaccgcgtggcgtacaacgtcggc | |
| | gggcagatggccaccaacaaccagagctccaccactgccc | |
| | ccgcgaccggcacgtacaacctccaggaaatcgtgcccgg | |
| | cagcgtgtggatggagagggacgtgtacctccaaggaccc | |
| | atctgggccaagatcccagagacgggggcgcactttcacc | |
| | cctctccggccatgggcggattcggactcaaacacccacc | |
| | gcccatgatgctcatcaagaacacgcctgtgcccggaaat | |
| | atcaccagcttctcggacgtgcccgtcagcagcttcatca | |
| | cccagtacagcaccgggcaggtcaccgtggagatggagtg | |
| | ggagctcaagaaggaaaactccaagaggtggaacccagag | |
| | atccagtacacaaacaactacaacgacccccagtttgtgg | |
| | actttgccccggacagcaccggggaatacagaaccaccag | |
| | acctatcggaacccgataccttacccgacccctttaaccc | |
| | attcatgtcgcataccctcaataaaccgtgtattcgtgtc | |
| | agtaaaatactgcctcttgtggtttggcgtaatcatggtc | |
| | atagctgtttcctgtgtgaaattgttatccgctcacaatt | |
| | ccacacaacatacgagccggaagcataaagtgtaaagcct | |
| | ggggtgcctaatgagtgagctaactcacattaattgcgtt | |
| | gcgctcactgcccgctttccagtcgggaaacctgtcgtgc | |
| | cagctgcattaatgaatcggccaacgcgcggggagaggcg | |
| | gtttgcgtattgggcgctcttccgcttcctcgctcactga | |
| | ctcgctgcgctcggtcgttcggctgcggcgagcggtatca | |
| | gctcactcaaaggcggtaatacggttatccacagaatcag | |
| | gggataacgcaggaaagaacatgtgagcaaaaggccagca | |
| | aaaggccaggaaccgtaaaaaggccgcgttgctggcgttt | |
| | ttccataggctccgcccccctgacgagcatcacaaaaatc | |
| | gacgctcaagtcagaggtggcgaaacccgacaggactata | |
| | aagataccaggcgtttccccctggaagctccctcgtgcgc | |
| | tctcctgttccgaccctgccgcttaccggatacctgtccg | |
| | cctttctcccttcgggaagcgtggcgctttctcatagctc | |
| | acgctgtaggtatctcagttcggtgtaggtcgttcgctcc | |
| | aagctgggctgtgtgcacgaaccccccgttcagcccgacc | |
| | gctgcgccttatccggtaactatcgtcttgagtccaaccc | |
| | ggtaagacacgacttatcgccactggcagcagccactggt | |
| | aacaggattagcagagcgaggtatgtaggcggtgctacag | |
| | agttcttgaagtggtggcctaactacggctacactagaag | |
| | aacagtatttggtatctgcgctctgctgaagccagttacc | |
| | ttcggaaaaagagttggtagctcttgatccggcaaacaaa | |
| | ccaccgctggtagcggtggtttttttgtttgcaagcagca | |
| | gattacgcgcagaaaaaaaggatctcaagaagatcctttg | |
| | atcttttctacggggtctgacgctcagtggaacgaaaact | |
| | cacgttaagggattttggtcatgagattatcaaaaaggat | |
| | cttcacctagatccttttaaattaaaaatgaagttttaaa | |
| | tcaatctaaagtatatatgagtaaacttggtctgacagtt | |
| | accaatgcttaatcagtgaggcacctatctcagcgatctg | |
| | tctatttcgttcatccatagttgcctgactccccgtcgtg | |
| | tagataactacgatacgggagggcttaccatctggcccca | |
| | gtgctgcaatgataccgcgagacccacgctcaccggctcc | |
| | agatttatcagcaataaaccagccagccggaagggccgag | |
| | cgcagaagtggtcctgcaactttatccgcctccatccagt | |
| | ctattaattgttgccgggaagctagagtaagtagttcgcc | |
| | agttaatagtttgcgcaacgttgttgccattgctacaggc | |
| | atcgtggtgtcacgctcgtcgtttggtatggcttcattca | |
| | gctccggttcccaacgatcaaggcgagttacatgatcccc | |
| | catgttgtgcaaaaaagcggttagctccttcggtcctccg | |
| | atcgttgtcagaagtaagttggccgcagtgttatcactca | |
| | tggttatggcagcactgcataattctcttactgtcatgcc | |
| | atccgtaagatgcttttctgtgactggtgagtactcaacc | |
| | aagtcattctgagaatagtgtatgcggcgaccgagttgct | |
| | cttgcccggcgtcaatacgggataataccgcgccacatag | |
| | cagaactttaaaagtgctcatcattggaaaacgttcttcg | |
| | gggcgaaaactctcaaggatcttaccgctgttgagatcca | |
| | gttcgatgtaacccactcgtgcacccaactgatcttcagc | |
| | atcttttactttcaccagcgtttctgggtgagcaaaaaca | |
| | ggaaggcaaaatgccgcaaaaaagggaataagggcgacac | |
| | ggaaatgttgaatactcatactcttcctttttcaatatta | |
| | ttgaagcatttatcagggttattgtctcatgagcggatac | |
| | atatttgaatgtatttagaaaaataaacaaataggggttc | |
| | cgcgcacatttccccgaaaagtgccacctgacgtctaaga | |
| | aaccattattatcatgacattaacctataaaaataggcgt | |
| | atcacgaggccctttcgtc | |
| A rep/cap | tcgcgcgtttcggtgatgacggtgaaaacctctgacacat | 5 |
| plasmid with | gcagctcccggagacggtcacagcttgtctgtaagcggat | |
| modified AAV9 | gccgggagcagacaagcccgtcagggcgcgtcagcgggtg | |
| cap: | ttggcgggtgtcggggctggcttaactatgcggcatcaga | |
| pZC375_AAV9 | gcagattgtactgagagtgcaccatatgcggtgtgaaata | |
| .VR-VIII- | ccgcacagatgcgtaaggagaaaataccgcatcaggcgcc | |
| NSTYLG VR- | attcgccattcaggctgcgcaactgttgggaagggcgatc | |
| IV-KGSGQNQ | ggtgcgggcctcttcgctattacgccagctggcgaaaggg | |
| | ggatgtgctgcaaggcgattaagttgggtaacgccagggt | |
| | tttcccagtcacgacgttgtaaaacgacggccagtgaatt | |
| | gacgcgtattgggatgtagttaatgattaacccgccatgc | |
| | tacttatctacgtagccatgctctaggaagatcggaattc | |
| | gccctattagagtcctgtattagaggtcacgtgagtgttt | |
| | tgcgacattttgcgacaccatgtggtcacgctgggtattt | |
| | aagcccgagtgagcacgcagggtctccattttgaagcggg | |
| | aggtttgaacgcgcagccaccacggcggggttttacgaga | |
| | ttgtgattaaggtccccagcgaccttgacgagcatctgcc | |
| | cggcatttctgacagctttgtgaactgggggccgagaagg | |
| | aatgggagttgccgccagattctgacatggatctgaatct | |
| | gattgagcaggcacccctgaccgtggccgagaagctgcag | |
| | cgcgactttctgacggaatggcgccgtgtgagtaaggccc | |
| | cggaggcccttttctttgtgcaatttgagaagggagagag | |
| | ctacttccacatgcacgtgctcgtggaaaccaccggggtg | |
| | aaatccatggttttgggacgtttcctgagtcagattcgcg | |
| | aaaaactgattcagagaatttaccgcgggatcgagccgac | |
| | tttgccaaactggttcgcggtcacaaagaccagaaatggc | |
| | gccggaggcgggaacaaggtggtggatgagtgctacatcc | |
| | ccaattacttgctccccaaaacccagcctgagctccagtg | |
| | ggcgtggactaatatggaacagtatttaagcgcctgtttg | |
| | aatctcacggagcgtaaacggttggtggcgcagcatctga | |
| | cgcacgtgtcgcagacgcaggagcagaacaaagagaatca | |
| | gaatcccaattctgatgcgccggtgatcagatcaaaaact | |
| | tcagccaggtacatggagctggtcgggtggctcgtggaca | |
| | aggggattacctcggagaagcagtggatccaggaggacca | |
| | ggcctcatacatctccttcaatgcggcctccaactcgcgg | |
| | tcccaaatcaaggctgccttggacaatgcgggaaagatta | |
| | tgagcctgactaaaaccgcccccgactacctgggggccag | |
| | cagcccgtggaggacatttccagcaatcggatttataaaa | |
| | ttttggaactaaacgggtacgatccccaatatgcggcttc | |
| | cgtctttctgggatgggccacgaaaaagttcggcaagagg | |
| | aacaccatctggctgtttgggcctgcaactaccgggaaga | |
| | ccaacatcgcggaggccatagcccacactgtgcccttcta | |
| | cgggtgcgtaaactggaccaatgagaactttcccttcaac | |
| | gactgtgtcgacaagatggtgatctggtgggaggagggga | |
| | agatgaccgccaaggtcgtggagtcggccaaagccattct | |
| | cggaggaagcaaggtgcgcgtggaccagaaatgcaagtcc | |
| | tcggcccagatagacccgactcccgtgatcgtcacctcca | |
| | acaccaacatgtgcgccgtgattgacgggaactcaacgac | |
| | cttcgaacaccagcagccgttgcaagaccggatgttcaaa | |
| | tttgaactcacccgccgtctggatcatgactttgggaagg | |
| | tcaccaagcaggaagtcaaagactttttccggtgggcaaa | |
| | ggatcacgtggttgaggtggagcatgaattctacgtcaaa | |
| | aagggtggagccaagaaaagacccgcccccagtgacgcag | |
| | atataagtgagcccaaacgggtgcgcgagtcagttgcgca | |
| | gccatcgacgtcagacgcggaagcttcgatcaactacgca | |
| | gacaggtaccaaaacaaatgttctcgtcacgtgggcatga | |
| | atctgatgctgtttccctgcagacaatgcgagagaatgaa | |
| | tcagaattcaaatatctgcttcactcacggacagaaagac | |
| | tgtttagagtgctttcccgtgtcagaatctcaacccgttt | |
| | ctgtcgtcaaaaaggcgtatcagaaactgtgctacattca | |
| | tcatatcatgggaaaggtgccagacgcttgcactgcctgc | |
| | gatctggtcaatgtggatttggatgactgcatctttgaac | |
| | aataaatgacttaaaccaggtatggctgccgatggttatc | |
| | ttccagattggctcgaggacaaccttagtgaaggaattcg | |
| | cgagtggtgggctttgaaacctggagcccctcaacccaag | |
| | gcaaatcaacaacatcaagacaacgctcgaggtcttgtgc | |
| | ttccgggttacaaataccttggacccggcaacggactcga | |
| | caagggggagccggtcaacgcagcagacgcggcggccctc | |
| | gagcacgacaaggcctacgaccagcagctcaaggccggag | |
| | acaacccgtacctcaagtacaaccacgccgacgccgagtt | |
| | ccaggagcggctcaaagaagatacgtcttttgggggcaac | |
| | ctcgggcgagcagtcttccaggccaaaaagaggcttcttg | |
| | aacctcttggtctggttgaggaagcggctaagacggctcc | |
| | tggaaagaagaggcctgtagagcagtctcctcaggaaccg | |
| | gactcctccgcgggtattggcaaatcgggtgcacagcccg | |
| | ctaaaaagagactcaatttcggtcagactggcgacacaga | |
| | gtcagtcccagaccctcaaccaatcggagaacctcccgca | |
| | gccccctcaggtgtgggatctcttacaatggcttcaggtg | |
| | gtggcgcaccagtggcagacaataacgaaggtgccgatgg | |
| | agtgggtagttcctcgggaaattggcattgcgattcccaa | |
| | tggctgggggacagagtcatcaccaccagcacccgaacct | |
| | gggccctgcccacctacaacaatcacctctacaagcaaat | |
| | ctccaacagcacatctggaggatcttcaaatgacaacgcc | |
| | tacttcggctacagcaccccctgggggtattttgacttca | |
| | acagattccactgccacttctcaccacgtgactggcagcg | |
| | actcatcaacaacaactggggattccggcctaagcgactc | |
| | aacttcaagctcttcaacattcaggtcaaagaggttacgg | |
| | acaacaatggagtcaagaccatcgccaataaccttaccag | |
| | cacggtccaggtcttcacggactcagactatcagctcccg | |
| | tacgtgctcgggtcggctcacgagggctgcctcccgccgt | |
| | tcccagcggacgttttcatgattcctcagtacgggtatct | |
| | gacgcttaatgatggaagccaggccgtgggtcgttcgtcc | |
| | ttttactgcctggaatatttcccgtcgcaaatgctaagaa | |
| | cgggtaacaacttccagttcagctacgagtttgagaacgt | |
| | acctttccatagcagctacgctcacagccaaagcctggac | |
| | cgactaatgaatccactcatcgaccaatacttgtactatc | |
| | tctcaaagactattaagggctccggccagaatcagcaaac | |
| | gctaaaattcagtgtggccggacccagcaacatggctgtc | |
| | cagggaagaaactacatacctggacccagctaccgacaac | |
| | aacgtgtctcaaccactgtgactcaaaacaacaacagcga | |
| | atttgcttggcctggagcttcttcttgggctctcaatgga | |
| | cgtaatagcttgatgaatcctggacctgctatggccagcc | |
| | acaaagaaggagaggaccgtttctttcctttgtctggatc | |
| | tttaatttttggcaaacaaggaactggaagagacaacgtg | |
| | gatgcggacaaagtcatgataaccaacgaagaagaaatta | |
| | aaactactaacccggtagcaacggagtcctatggacaagt | |
| | ggccacaaaccacaattccacctacctgggcgcgcagacc | |
| | ggctgggttcaaaaccaaggaatacttccgggtatggttt | |
| | ggcaggacagagatgtgtacctgcaaggacccatttgggc | |
| | caaaattcctcacacggacggcaactttcacccttctccg | |
| | ctgatgggagggtttggaatgaagcacccgcctcctcaga | |
| | tcctcatcaaaaacacacctgtacctgcggatcctccaac | |
| | ggccttcaacaaggacaagctgaactctttcatcacccag | |
| | tattctactggccaagtcagcgtggagatcgagtgggagc | |
| | tgcagaaggaaaacagcaagcgctggaacccggagatcca | |
| | gtacacttccaactattacaagtctaataatgttgaattt | |
| | gctgttaatactgaaggtgtatatagtgaaccccgcccca | |
| | ttggcaccagatacctgactcgtaatctgtaattgcttgt | |
| | taatcaataaaccgtttaattcgtttcagttgaactttgg | |
| | tctctgcgaaggcgcgccataacttcgttggcgtaatcat | |
| | ggtcatagctgtttcctgtgtgaaattgttatccgctcac | |
| | aattccacacaacatacgagccggaagcataaagtgtaaa | |
| | gcctggggtgcctaatgagtgagctaactcacattaattg | |
| | cgttgcgctcactgcccgctttccagtcgggaaacctgtc | |
| | gtgccagctgcattaatgaatcggccaacgcgcggggaga | |
| | ggcggtttgcgtattgggcgctcttccgcttcctcgctca | |
| | ctgactcgctgcgctcggtcgttcggctgcggcgagcggt | |
| | atcagctcactcaaaggcggtaatacggttatccacagaa | |
| | tcaggggataacgcaggaaagaacatgtgagcaaaaggcc | |
| | agcaaaaggccaggaaccgtaaaaaggccgcgttgctggc | |
| | gtttttccataggctccgcccccctgacgagcatcacaaa | |
| | aatcgacgctcaagtcagaggtggcgaaacccgacaggac | |
| | tataaagataccaggcgtttccccctggaagctccctcgt | |
| | gcgctctcctgttccgaccctgccgcttaccggatacctg | |
| | tccgcctttctcccttcgggaagcgtggcgctttctcata | |
| | gctcacgctgtaggtatctcagttcggtgtaggtcgttcg | |
| | ctccaagctgggctgtgtgcacgaaccccccgttcagccc | |
| | gaccgctgcgccttatccggtaactatcgtcttgagtcca | |
| | acccggtaagacacgacttatcgccactggcagcagccac | |
| | tggtaacaggattagcagagcgaggtatgtaggcggtgct | |
| | acagagttcttgaagtggtggcctaactacggctacacta | |
| | gaagaacagtatttggtatctgcgctctgctgaagccagt | |
| | taccttcggaaaaagagttggtagctcttgatccggcaaa | |
| | caaaccaccgctggtagcggtggtttttttgtttgcaagc | |
| | agcagattacgcgcagaaaaaaaggatctcaagaagatcc | |
| | tttgatcttttctacggggtctgacgctcagtggaacgaa | |
| | aactcacgttaagggattttggtcatgagattatcaaaaa | |
| | ggatcttcacctagatccttttaaattaaaaatgaagttt | |
| | taaatcaatctaaagtatatatgagtaaacttggtctgac | |
| | agttaccaatgcttaatcagtgaggcacctatctcagcga | |
| | tctgtctatttcgttcatccatagttgcctgactccccgt | |
| | cgtgtagataactacgatacgggagggcttaccatctggc | |
| | cccagtgctgcaatgataccgcgagacccacgctcaccgg | |
| | ctccagatttatcagcaataaaccagccagccggaagggc | |
| | cgagcgcagaagtggtcctgcaactttatccgcctccatc | |
| | cagtctattaattgttgccgggaagctagagtaagtagtt | |
| | cgccagttaatagtttgcgcaacgttgttgccattgctac | |
| | aggcatcgtggtgtcacgctcgtcgtttggtatggcttca | |
| | ttcagctccggttcccaacgatcaaggcgagttacatgat | |
| | cccccatgttgtgcaaaaaagcggttagctccttcggtcc | |
| | tccgatcgttgtcagaagtaagttggccgcagtgttatca | |
| | ctcatggttatggcagcactgcataattctcttactgtca | |
| | tgccatccgtaagatgcttttctgtgactggtgagtactc | |
| | aaccaagtcattctgagaatagtgtatgcggcgaccgagt | |
| | tgctcttgcccggcgtcaatacgggataataccgcgccac | |
| | atagcagaactttaaaagtgctcatcattggaaaacgttc | |
| | ttcggggcgaaaactctcaaggatcttaccgctgttgaga | |
| | tccagttcgatgtaacccactcgtgcacccaactgatctt | |
| | cagcatcttttactttcaccagcgtttctgggtgagcaaa | |
| | aacaggaaggcaaaatgccgcaaaaaagggaataagggcg | |
| | acacggaaatgttgaatactcatactcttcctttttcaat | |
| | attattgaagcatttatcagggttattgtctcatgagcgg | |
| | atacatatttgaatgtatttagaaaaataaacaaataggg | |
| | gttccgcgcacatttccccgaaaagtgccacctgacgtct | |
| | aagaaaccattattatcatgacattaacctataaaaatag | |
| | gcgtatcacgaggccctttcgtc | |
| A rep/cap | tcgcgcgtttcggtgatgacggtgaaaacctctgacacat | 6 |
| plasmid with | gcagctcccggagacggtcacagcttgtctgtaagcggat | |
| modified AAV9 | gccgggagcagacaagcccgtcagggcgcgtcagcgggtg | |
| cap: | ttggcgggtgtcggggctggcttaactatgcggcatcaga | |
| pZC401_AAV9 | gcagattgtactgagagtgcaccatatgcggtgtgaaata | |
| VR-VIII- | ccgcacagatgcgtaaggagaaaataccgcatcaggcgcc | |
| MMTTAR | attcgccattcaggctgcgcaactgttgggaagggcgatc | |
| | ggtgcgggcctcttcgctattacgccagctggcgaaaggg | |
| | ggatgtgctgcaaggcgattaagttgggtaacgccagggt | |
| | tttcccagtcacgacgttgtaaaacgacggccagtgaatt | |
| | gacgcgtattgggatgtagttaatgattaacccgccatgc | |
| | tacttatctacgtagccatgctctaggaagatcggaattc | |
| | gccctattagagtcctgtattagaggtcacgtgagtgttt | |
| | tgcgacattttgcgacaccatgtggtcacgctgggtattt | |
| | aagcccgagtgagcacgcagggtctccattttgaagcggg | |
| | aggtttgaacgcgcagccaccacggcggggttttacgaga | |
| | ttgtgattaaggtccccagcgaccttgacgagcatctgcc | |
| | cggcatttctgacagctttgtgaactgggtggccgagaag | |
| | gaatgggagttgccgccagattctgacatggatctgaatc | |
| | tgattgagcaggcacccctgaccgtggccgagaagctgca | |
| | gcgcgactttctgacggaatggcgccgtgtgagtaaggcc | |
| | ccggaggcccttttctttgtgcaatttgagaagggagaga | |
| | gctacttccacatgcacgtgctcgtggaaaccaccggggt | |
| | gaaatccatggttttgggacgtttcctgagtcagattcgc | |
| | gaaaaactgattcagagaatttaccgcgggatcgagccga | |
| | ctttgccaaactggttcgcggtcacaaagaccagaaatgg | |
| | cgccggaggcgggaacaaggtggtggatgagtgctacatc | |
| | cccaattacttgctccccaaaacccagcctgagctccagt | |
| | gggcgtggactaatatggaacagtatttaagcgcctgttt | |
| | gaatctcacggagcgtaaacggttggtggcgcagcatctg | |
| | acgcacgtgtcgcagacgcaggagcagaacaaagagaatc | |
| | agaatcccaattctgatgcgccggtgatcagatcaaaaac | |
| | ttcagccaggtacatggagctggtcgggtggctcgtggac | |
| | aaggggattacctcggagaagcagtggatccaggaggacc | |
| | aggcctcatacatctccttcaatgcggcctccaactcgcg | |
| | gtcccaaatcaaggctgccttggacaatgcgggaaagatt | |
| | atgagcctgactaaaaccgcccccgactacctggtgggcc | |
| | agcagcccgtggaggacatttccagcaatcggatttataa | |
| | aattttggaactaaacgggtacgatccccaatatgcggct | |
| | tccgtctttctgggatgggccacgaaaaagttcggcaaga | |
| | ggaacaccatctggctgtttgggcctgcaactaccgggaa | |
| | gaccaacatcgcggaggccatagcccacactgtgcccttc | |
| | tacgggtgcgtaaactggaccaatgagaactttcccttca | |
| | acgactgtgtcgacaagatggtgatctggtgggaggaggg | |
| | gaagatgaccgccaaggtcgtggagtcggccaaagccatt | |
| | ctcggaggaagcaaggtgcgcgtggaccagaaatgcaagt | |
| | cctcggcccagatagacccgactcccgtgatcgtcacctc | |
| | caacaccaacatgtgcgccgtgattgacgggaactcaacg | |
| | accttcgaacaccagcagccgttgcaagaccggatgttca | |
| | aatttgaactcacccgccgtctggatcatgactttgggaa | |
| | ggtcaccaagcaggaagtcaaagactttttccggtgggca | |
| | aaggatcacgtggttgaggtggagcatgaattctacgtca | |
| | aaaagggtggagccaagaaaagacccgcccccagtgacgc | |
| | agatataagtgagcccaaacgggtgcgcgagtcagttgcg | |
| | cagccatcgacgtcagacgcggaagcttcgatcaactacg | |
| | cagacaggtaccaaaacaaatgttctcgtcacgtgggcat | |
| | gaatctgatgctgtttccctgcagacaatgcgagagaatg | |
| | aatcagaattcaaatatctgcttcactcacggacagaaag | |
| | actgtttagagtgctttcccgtgtcagaatctcaacccgt | |
| | ttctgtcgtcaaaaaggcgtatcagaaactgtgctacatt | |
| | catcatatcatgggaaaggtgccagacgcttgcactgcct | |
| | gcgatctggtcaatgtggatttggatgactgcatctttga | |
| | acaataaatgacttaaaccaggtatggctgccgatggtta | |
| | tcttccagattggctcgaggacaaccttagtgaaggaatt | |
| | cgcgagtggtgggctttgaaacctggagcccctcaaccca | |
| | aggcaaatcaacaacatcaagacaacgctcgaggtcttgt | |
| | gcttccgggttacaaataccttggacccggcaacggactc | |
| | gacaagggggagccggtcaacgcagcagacgcggcggccc | |
| | tcgagcacgacaaggcctacgaccagcagctcaaggccgg | |
| | agacaacccgtacctcaagtacaaccacgccgacgccgag | |
| | ttccaggagcggctcaaagaagatacgtcttttgggggca | |
| | acctcgggcgagcagtcttccaggccaaaaagaggcttct | |
| | tgaacctcttggtctggttgaggaagcggctaagacggct | |
| | cctggaaagaagaggcctgtagagcagtctcctcaggaac | |
| | cggactcctccgcgggtattggcaaatcgggtgcacagcc | |
| | cgctaaaaagagactcaatttcggtcagactggcgacaca | |
| | gagtcagtcccagaccctcaaccaatcggagaacctcccg | |
| | cagccccctcaggtgtgggatctcttacaatggcttcagg | |
| | tggtggcgcaccagtggcagacaataacgaaggtgccgat | |
| | ggagtgggtagttcctcgggaaattggcattgcgattccc | |
| | aatggctgggggacagagtcatcaccaccagcacccgaac | |
| | ctgggccctgcccacctacaacaatcacctctacaagcaa | |
| | atctccaacagcacatctggaggatcttcaaatgacaacg | |
| | cctacttcggctacagcaccccctgggggtattttgactt | |
| | caacagattccactgccacttctcaccacgtgactggcag | |
| | cgactcatcaacaacaactggggattccggcctaagcgac | |
| | tcaacttcaagctcttcaacattcaggtcaaagaggttac | |
| | ggacaacaatggagtcaagaccatcgccaataaccttacc | |
| | agcacggtccaggtcttcacggactcagactatcagctcc | |
| | cgtacgtgctcgggtcggctcacgagggctgcctcccgcc | |
| | gttcccagcggacgttttcatgattcctcagtacgggtat | |
| | ctgacgcttaatgatggaagccaggccgtgggtcgttcgt | |
| | ccttttactgcctggaatatttcccgtcgcaaatgctaag | |
| | aacgggtaacaacttccagttcagctacgagtttgagaac | |
| | gtacctttccatagcagctacgctcacagccaaagcctgg | |
| | accgactaatgaatccactcatcgaccaatacttgtacta | |
| | tctctcaaagactattaacggttctggacagaatcaacaa | |
| | acgctaaaattcagtgtggccggacccagcaacatggctg | |
| | tccagggaagaaactacatacctggacccagctaccgaca | |
| | acaacgtgtctcaaccactgtgactcaaaacaacaacagc | |
| | gaatttgcttggcctggagcttcttcttgggctctcaatg | |
| | gacgtaatagcttgatgaatcctggacctgctatggccag | |
| | ccacaaagaaggagaggaccgtttctttcctttgtctgga | |
| | tctttaatttttggcaaacaaggaactggaagagacaacg | |
| | tggatgcggacaaagtcatgataaccaacgaagaagaaat | |
| | taaaactactaacccggtagcaacggagtcctatggacaa | |
| | gtggccacaaaccatatgatgaccaccgccagagcgcaga | |
| | ccggctgggttcaaaaccaaggaatacttccgggtatggt | |
| | ttggcaggacagagatgtgtacctgcaaggacccatttgg | |
| | gccaaaattcctcacacggacggcaactttcacccttctc | |
| | cgctgatgggagggtttggaatgaagcacccgcctcctca | |
| | gatcctcatcaaaaacacacctgtacctgcggatcctcca | |
| | acggccttcaacaaggacaagctgaactctttcatcaccc | |
| | agtattctactggccaagtcagcgtggagatcgagtggga | |
| | gctgcagaaggaaaacagcaagcgctggaacccggagatc | |
| | cagtacacttccaactattacaagtctaataatgttgaat | |
| | ttgctgttaatactgaaggtgtatatagtgaaccccgccc | |
| | cattggcaccagatacctgactcgtaatctgtaattgctt | |
| | gttaatcaataaaccgtttaattcgtttcagttgaacttt | |
| | ggtctctgcgaaggcgcgccataacttcgttggcgtaatc | |
| | atggtcatagctgtttcctgtgtgaaattgttatccgctc | |
| | acaattccacacaacatacgagccggaagcataaagtgta | |
| | aagcctggggtgcctaatgagtgagctaactcacattaat | |
| | tgcgttgcgctcactgcccgctttccagtcgggaaacctg | |
| | tcgtgccagctgcattaatgaatcggccaacgcgcgggga | |
| | gaggcggtttgcgtattgggcgctcttccgcttcctcgct | |
| | cactgactcgctgcgctcggtcgttcggctgcggcgagcg | |
| | gtatcagctcactcaaaggcggtaatacggttatccacag | |
| | aatcaggggataacgcaggaaagaacatgtgagcaaaagg | |
| | ccagcaaaaggccaggaaccgtaaaaaggccgcgttgctg | |
| | gcgtttttccataggctccgcccccctgacgagcatcaca | |
| | aaaatcgacgctcaagtcagaggtggcgaaacccgacagg | |
| | actataaagataccaggcgtttccccctggaagctccctc | |
| | gtgcgctctcctgttccgaccctgccgcttaccggatacc | |
| | tgtccgcctttctcccttcgggaagcgtggcgctttctca | |
| | tagctcacgctgtaggtatctcagttcggtgtaggtcgtt | |
| | cgctccaagctgggctgtgtgcacgaaccccccgttcagc | |
| | ccgaccgctgcgccttatccggtaactatcgtcttgagtc | |
| | caacccggtaagacacgacttatcgccactggcagcagcc | |
| | actggtaacaggattagcagagcgaggtatgtaggcggtg | |
| | ctacagagttcttgaagtggtggcctaactacggctacac | |
| | tagaagaacagtatttggtatctgcgctctgctgaagcca | |
| | gttaccttcggaaaaagagttggtagctcttgatccggca | |
| | aacaaaccaccgctggtagcggtggtttttttgtttgcaa | |
| | gcagcagattacgcgcagaaaaaaaggatctcaagaagat | |
| | cctttgatcttttctacggggtctgacgctcagtggaacg | |
| | aaaactcacgttaagggattttggtcatgagattatcaaa | |
| | aaggatcttcacctagatccttttaaattaaaaatgaagt | |
| | tttaaatcaatctaaagtatatatgagtaaacttggtctg | |
| | acagttaccaatgcttaatcagtgaggcacctatctcagc | |
| | gatctgtctatttcgttcatccatagttgcctgactcccc | |
| | gtcgtgtagataactacgatacgggagggcttaccatctg | |
| | gccccagtgctgcaatgataccgcgagacccacgctcacc | |
| | ggctccagatttatcagcaataaaccagccagccggaagg | |
| | gccgagcgcagaagtggtcctgcaactttatccgcctcca | |
| | tccagtctattaattgttgccgggaagctagagtaagtag | |
| | ttcgccagttaatagtttgcgcaacgttgttgccattgct | |
| | acaggcatcgtggtgtcacgctcgtcgtttggtatggctt | |
| | cattcagctccggttcccaacgatcaaggcgagttacatg | |
| | atcccccatgttgtgcaaaaaagcggttagctccttcggt | |
| | cctccgatcgttgtcagaagtaagttggccgcagtgttat | |
| | cactcatggttatggcagcactgcataattctcttactgt | |
| | catgccatccgtaagatgcttttctgtgactggtgagtac | |
| | tcaaccaagtcattctgagaatagtgtatgcggcgaccga | |
| | gttgctcttgcccggcgtcaatacgggataataccgcgcc | |
| | acatagcagaactttaaaagtgctcatcattggaaaacgt | |
| | tcttcggggcgaaaactctcaaggatcttaccgctgttga | |
| | gatccagttcgatgtaacccactcgtgcacccaactgatc | |
| | ttcagcatcttttactttcaccagcgtttctgggtgagca | |
| | aaaacaggaaggcaaaatgccgcaaaaaagggaataaggg | |
| | cgacacggaaatgttgaatactcatactcttcctttttca | |
| | atattattgaagcatttatcagggttattgtctcatgagc | |
| | ggatacatatttgaatgtatttagaaaaataaacaaatag | |
| | gggttccgcgcacatttccccgaaaagtgccacctgacgt | |
| | ctaagaaaccattattatcatgacattaacctataaaaat | |
| | aggcgtatcacgaggccctttcgtc | |
| A rep/cap | tcgcgcgtttcggtgatgacggtgaaaacctctgacacat | 7 |
| plasmid with | gcagctcccggagacggtcacagcttgtctgtaagcggat | |
| modified AAV9 | gccgggagcagacaagcccgtcagggcgcgtcagcgggtg | |
| cap: | ttggcgggtgtcggggctggcttaactatgcggcatcaga | |
| pZC428_AAV9 | gcagattgtactgagagtgcaccatatgcggtgtgaaata | |
| VR-VIII- | ccgcacagatgcgtaaggagaaaataccgcatcaggcgcc | |
| NSTYLG | attcgccattcaggctgcgcaactgttgggaagggcgatc | |
| | ggtgcgggcctcttcgctattacgccagctggcgaaaggg | |
| | ggatgtgctgcaaggcgattaagttgggtaacgccagggt | |
| | tttcccagtcacgacgttgtaaaacgacggccagtgaatt | |
| | gacgcgtattgggatgtagttaatgattaacccgccatgc | |
| | tacttatctacgtagccatgctctaggaagatcggaattc | |
| | gccctattagagtcctgtattagaggtcacgtgagtgttt | |
| | tgcgacattttgcgacaccatgtggtcacgctgggtattt | |
| | aagcccgagtgagcacgcagggtctccattttgaagcggg | |
| | aggtttgaacgcgcagccaccacggcggggttttacgaga | |
| | ttgtgattaaggtccccagcgaccttgacgagcatctgcc | |
| | cggcatttctgacagctttgtgaactgggtggccgagaag | |
| | gaatgggagttgccgccagattctgacatggatctgaatc | |
| | tgattgagcaggcacccctgaccgtggccgagaagctgca | |
| | gcgcgactttctgacggaatggcgccgtgtgagtaaggcc | |
| | ccggaggcccttttctttgtgcaatttgagaagggagaga | |
| | gctacttccacatgcacgtgctcgtggaaaccaccggggt | |
| | gaaatccatggttttgggacgtttcctgagtcagattcgc | |
| | gaaaaactgattcagagaatttaccgcgggatcgagccga | |
| | ctttgccaaactggttcgcggtcacaaagaccagaaatgg | |
| | cgccggaggcgggaacaaggtggtggatgagtgctacatc | |
| | cccaattacttgctccccaaaacccagcctgagctccagt | |
| | gggcgtggactaatatggaacagtatttaagcgcctgttt | |
| | gaatctcacggagcgtaaacggttggtggcgcagcatctg | |
| | acgcacgtgtcgcagacgcaggagcagaacaaagagaatc | |
| | agaatcccaattctgatgcgccggtgatcagatcaaaaac | |
| | ttcagccaggtacatggagctggtcgggtggctcgtggac | |
| | aaggggattacctcggagaagcagtggatccaggaggacc | |
| | aggcctcatacatctccttcaatgcggcctccaactcgcg | |
| | gtcccaaatcaaggctgccttggacaatgcgggaaagatt | |
| | atgagcctgactaaaaccgcccccgactacctggtgggcc | |
| | agcagcccgtggaggacatttccagcaatcggatttataa | |
| | aattttggaactaaacgggtacgatccccaatatgcggct | |
| | tccgtctttctgggatgggccacgaaaaagttcggcaaga | |
| | ggaacaccatctggctgtttgggcctgcaactaccgggaa | |
| | gaccaacatcgcggaggccatagcccacactgtgcccttc | |
| | tacgggtgcgtaaactggaccaatgagaactttcccttca | |
| | acgactgtgtcgacaagatggtgatctggtgggaggaggg | |
| | gaagatgaccgccaaggtcgtggagtcggccaaagccatt | |
| | ctcggaggaagcaaggtgcgcgtggaccagaaatgcaagt | |
| | cctcggcccagatagacccgactcccgtgatcgtcacctc | |
| | caacaccaacatgtgcgccgtgattgacgggaactcaacg | |
| | accttcgaacaccagcagccgttgcaagaccggatgttca | |
| | aatttgaactcacccgccgtctggatcatgactttgggaa | |
| | ggtcaccaagcaggaagtcaaagactttttccggtgggca | |
| | aaggatcacgtggttgaggtggagcatgaattctacgtca | |
| | aaaagggtggagccaagaaaagacccgcccccagtgacgc | |
| | agatataagtgagcccaaacgggtgcgcgagtcagttgcg | |
| | cagccatcgacgtcagacgcggaagcttcgatcaactacg | |
| | cagacaggtaccaaaacaaatgttctcgtcacgtgggcat | |
| | gaatctgatgctgtttccctgcagacaatgcgagagaatg | |
| | aatcagaattcaaatatctgcttcactcacggacagaaag | |
| | actgtttagagtgctttcccgtgtcagaatctcaacccgt | |
| | ttctgtcgtcaaaaaggcgtatcagaaactgtgctacatt | |
| | catcatatcatgggaaaggtgccagacgcttgcactgcct | |
| | gcgatctggtcaatgtggatttggatgactgcatctttga | |
| | acaataaatgacttaaaccaggtatggctgccgatggtta | |
| | tcttccagattggctcgaggacaaccttagtgaaggaatt | |
| | cgcgagtggtgggctttgaaacctggagcccctcaaccca | |
| | aggcaaatcaacaacatcaagacaacgctcgaggtcttgt | |
| | gcttccgggttacaaataccttggacccggcaacggactc | |
| | gacaagggggagccggtcaacgcagcagacgcggcggccc | |
| | tcgagcacgacaaggcctacgaccagcagctcaaggccgg | |
| | agacaacccgtacctcaagtacaaccacgccgacgccgag | |
| | ttccaggagcggctcaaagaagatacgtcttttgggggca | |
| | acctcgggcgagcagtcttccaggccaaaaagaggcttct | |
| | tgaacctcttggtctggttgaggaagcggctaagacggct | |
| | cctggaaagaagaggcctgtagagcagtctcctcaggaac | |
| | cggactcctccgcgggtattggcaaatcgggtgcacagcc | |
| | cgctaaaaagagactcaatttcggtcagactggcgacaca | |
| | gagtcagtcccagaccctcaaccaatcggagaacctcccg | |
| | cagccccctcaggtgtgggatctcttacaatggcttcagg | |
| | tggtggcgcaccagtggcagacaataacgaaggtgccgat | |
| | ggagtgggtagttcctcgggaaattggcattgcgattccc | |
| | aatggctgggggacagagtcatcaccaccagcacccgaac | |
| | ctgggccctgcccacctacaacaatcacctctacaagcaa | |
| | atctccaacagcacatctggaggatcttcaaatgacaacg | |
| | cctacttcggctacagcaccccctgggggtattttgactt | |
| | caacagattccactgccacttctcaccacgtgactggcag | |
| | cgactcatcaacaacaactggggattccggcctaagcgac | |
| | tcaacttcaagctcttcaacattcaggtcaaagaggttac | |
| | ggacaacaatggagtcaagaccatcgccaataaccttacc | |
| | agcacggtccaggtcttcacggactcagactatcagctcc | |
| | cgtacgtgctcgggtcggctcacgagggctgcctcccgcc | |
| | gttcccagcggacgttttcatgattcctcagtacgggtat | |
| | ctgacgcttaatgatggaagccaggccgtgggtcgttcgt | |
| | ccttttactgcctggaatatttcccgtcgcaaatgctaag | |
| | aacgggtaacaacttccagttcagctacgagtttgagaac | |
| | gtacctttccatagcagctacgctcacagccaaagcctgg | |
| | accgactaatgaatccactcatcgaccaatacttgtacta | |
| | tctctcaaagactattaacggttctggacagaatcaacaa | |
| | acgctaaaattcagtgtggccggacccagcaacatggctg | |
| | tccagggaagaaactacatacctggacccagctaccgaca | |
| | acaacgtgtctcaaccactgtgactcaaaacaacaacagc | |
| | gaatttgcttggcctggagcttcttcttgggctctcaatg | |
| | gacgtaatagcttgatgaatcctggacctgctatggccag | |
| | ccacaaagaaggagaggaccgtttctttcctttgtctgga | |
| | tctttaatttttggcaaacaaggaactggaagagacaacg | |
| | tggatgcggacaaagtcatgataaccaacgaagaagaaat | |
| | taaaactactaacccggtagcaacggagtcctatggacaa | |
| | gtggccacaaaccacaattccacctacctgggcgcgcaga | |
| | ccggctgggttcaaaaccaaggaatacttccgggtatggt | |
| | ttggcaggacagagatgtgtacctgcaaggacccatttgg | |
| | gccaaaattcctcacacggacggcaactttcacccttctc | |
| | cgctgatgggagggtttggaatgaagcacccgcctcctca | |
| | gatcctcatcaaaaacacacctgtacctgcggatcctcca | |
| | acggccttcaacaaggacaagctgaactctttcatcaccc | |
| | agtattctactggccaagtcagcgtggagatcgagtggga | |
| | gctgcagaaggaaaacagcaagcgctggaacccggagatc | |
| | cagtacacttccaactattacaagtctaataatgttgaat | |
| | ttgctgttaatactgaaggtgtatatagtgaaccccgccc | |
| | cattggcaccagatacctgactcgtaatctgtaattgctt | |
| | gttaatcaataaaccgtttaattcgtttcagttgaacttt | |
| | ggtctctgcgaaggcgcgccataacttcgttggcgtaatc | |
| | atggtcatagctgtttcctgtgtgaaattgttatccgctc | |
| | acaattccacacaacatacgagccggaagcataaagtgta | |
| | aagcctggggtgcctaatgagtgagctaactcacattaat | |
| | tgcgttgcgctcactgcccgctttccagtcgggaaacctg | |
| | tcgtgccagctgcattaatgaatcggccaacgcgcgggga | |
| | gaggcggtttgcgtattgggcgctcttccgcttcctcgct | |
| | cactgactcgctgcgctcggtcgttcggctgcggcgagcg | |
| | gtatcagctcactcaaaggcggtaatacggttatccacag | |
| | aatcaggggataacgcaggaaagaacatgtgagcaaaagg | |
| | ccagcaaaaggccaggaaccgtaaaaaggccgcgttgctg | |
| | gcgtttttccataggctccgcccccctgacgagcatcaca | |
| | aaaatcgacgctcaagtcagaggtggcgaaacccgacagg | |
| | actataaagataccaggcgtttccccctggaagctccctc | |
| | gtgcgctctcctgttccgaccctgccgcttaccggatacc | |
| | tgtccgcctttctcccttcgggaagcgtggcgctttctca | |
| | tagctcacgctgtaggtatctcagttcggtgtaggtcgtt | |
| | cgctccaagctgggctgtgtgcacgaaccccccgttcagc | |
| | ccgaccgctgcgccttatccggtaactatcgtcttgagtc | |
| | caacccggtaagacacgacttatcgccactggcagcagcc | |
| | actggtaacaggattagcagagcgaggtatgtaggcggtg | |
| | ctacagagttcttgaagtggtggcctaactacggctacac | |
| | tagaagaacagtatttggtatctgcgctctgctgaagcca | |
| | gttaccttcggaaaaagagttggtagctcttgatccggca | |
| | aacaaaccaccgctggtagcggtggtttttttgtttgcaa | |
| | gcagcagattacgcgcagaaaaaaaggatctcaagaagat | |
| | cctttgatcttttctacggggtctgacgctcagtggaacg | |
| | aaaactcacgttaagggattttggtcatgagattatcaaa | |
| | aaggatcttcacctagatccttttaaattaaaaatgaagt | |
| | tttaaatcaatctaaagtatatatgagtaaacttggtctg | |
| | acagttaccaatgcttaatcagtgaggcacctatctcagc | |
| | gatctgtctatttcgttcatccatagttgcctgactcccc | |
| | gtcgtgtagataactacgatacgggagggcttaccatctg | |
| | gccccagtgctgcaatgataccgcgagacccacgctcacc | |
| | ggctccagatttatcagcaataaaccagccagccggaagg | |
| | gccgagcgcagaagtggtcctgcaactttatccgcctcca | |
| | tccagtctattaattgttgccgggaagctagagtaagtag | |
| | ttcgccagttaatagtttgcgcaacgttgttgccattgct | |
| | acaggcatcgtggtgtcacgctcgtcgtttggtatggctt | |
| | cattcagctccggttcccaacgatcaaggcgagttacatg | |
| | atcccccatgttgtgcaaaaaagcggttagctccttcggt | |
| | cctccgatcgttgtcagaagtaagttggccgcagtgttat | |
| | cactcatggttatggcagcactgcataattctcttactgt | |
| | catgccatccgtaagatgcttttctgtgactggtgagtac | |
| | tcaaccaagtcattctgagaatagtgtatgcggcgaccga | |
| | gttgctcttgcccggcgtcaatacgggataataccgcgcc | |
| | acatagcagaactttaaaagtgctcatcattggaaaacgt | |
| | tcttcggggcgaaaactctcaaggatcttaccgctgttga | |
| | gatccagttcgatgtaacccactcgtgcacccaactgatc | |
| | ttcagcatcttttactttcaccagcgtttctgggtgagca | |
| | aaaacaggaaggcaaaatgccgcaaaaaagggaataaggg | |
| | cgacacggaaatgttgaatactcatactcttcctttttca | |
| | atattattgaagcatttatcagggttattgtctcatgagc | |
| | ggatacatatttgaatgtatttagaaaaataaacaaatag | |
| | gggttccgcgcacatttccccgaaaagtgccacctgacgt | |
| | ctaagaaaccattattatcatgacattaacctataaaaat | |
| | aggcgtatcacgaggccctttcgtc | |
| A rep/cap | tcgcgcgtttcggtgatgacggtgaaaacctctgacacat | 8 |
| plasmid with | gcagctcccggagacggtcacagcttgtctgtaagcggat | |
| modified AAV9 | gccgggagcagacaagcccgtcagggcgcgtcagcgggtg | |
| cap: | ttggcgggtgtcggggctggcttaactatgcggcatcaga | |
| pZC478_AAV9 | gcagattgtactgagagtgcaccatatgcggtgtgaaata | |
| .VR-VIII- | ccgcacagatgcgtaaggagaaaataccgcatcaggcgcc | |
| CSTSIR | attcgccattcaggctgcgcaactgttgggaagggcgatc | |
| | ggtgcgggcctcttcgctattacgccagctggcgaaaggg | |
| | ggatgtgctgcaaggcgattaagttgggtaacgccagggt | |
| | tttcccagtcacgacgttgtaaaacgacggccagtgaatt | |
| | gacgcgtattgggatgtagttaatgattaacccgccatgc | |
| | tacttatctacgtagccatgctctaggaagatcggaattc | |
| | gccctattagagtcctgtattagaggtcacgtgagtgttt | |
| | tgcgacattttgcgacaccatgtggtcacgctgggtattt | |
| | aagcccgagtgagcacgcagggtctccattttgaagcggg | |
| | aggtttgaacgcgcagccaccacggcggggttttacgaga | |
| | ttgtgattaaggtccccagcgaccttgacgagcatctgcc | |
| | cggcatttctgacagctttgtgaactgggtggccgagaag | |
| | gaatgggagttgccgccagattctgacatggatctgaatc | |
| | tgattgagcaggcacccctgaccgtggccgagaagctgca | |
| | gcgcgactttctgacggaatggcgccgtgtgagtaaggcc | |
| | ccggaggcccttttctttgtgcaatttgagaagggagaga | |
| | gctacttccacatgcacgtgctcgtggaaaccaccggggt | |
| | gaaatccatggttttgggacgtttcctgagtcagattcgc | |
| | gaaaaactgattcagagaatttaccgcgggatcgagccga | |
| | ctttgccaaactggttcgcggtcacaaagaccagaaatgg | |
| | cgccggaggcgggaacaaggtggtggatgagtgctacatc | |
| | cccaattacttgctccccaaaacccagcctgagctccagt | |
| | gggcgtggactaatatggaacagtatttaagcgcctgttt | |
| | gaatctcacggagcgtaaacggttggtggcgcagcatctg | |
| | acgcacgtgtcgcagacgcaggagcagaacaaagagaatc | |
| | agaatcccaattctgatgcgccggtgatcagatcaaaaac | |
| | ttcagccaggtacatggagctggtcgggtggctcgtggac | |
| | aaggggattacctcggagaagcagtggatccaggaggacc | |
| | aggcctcatacatctccttcaatgcggcctccaactcgcg | |
| | gtcccaaatcaaggctgccttggacaatgcgggaaagatt | |
| | atgagcctgactaaaaccgcccccgactacctggtgggcc | |
| | agcagcccgtggaggacatttccagcaatcggatttataa | |
| | aattttggaactaaacgggtacgatccccaatatgcggct | |
| | tccgtctttctgggatgggccacgaaaaagttcggcaaga | |
| | ggaacaccatctggctgtttgggcctgcaactaccgggaa | |
| | gaccaacatcgcggaggccatagcccacactgtgcccttc | |
| | tacgggtgcgtaaactggaccaatgagaactttcccttca | |
| | acgactgtgtcgacaagatggtgatctggtgggaggaggg | |
| | gaagatgaccgccaaggtcgtggagtcggccaaagccatt | |
| | ctcggaggaagcaaggtgcgcgtggaccagaaatgcaagt | |
| | cctcggcccagatagacccgactcccgtgatcgtcacctc | |
| | caacaccaacatgtgcgccgtgattgacgggaactcaacg | |
| | accttcgaacaccagcagccgttgcaagaccggatgttca | |
| | aatttgaactcacccgccgtctggatcatgactttgggaa | |
| | ggtcaccaagcaggaagtcaaagactttttccggtgggca | |
| | aaggatcacgtggttgaggtggagcatgaattctacgtca | |
| | aaaagggtggagccaagaaaagacccgcccccagtgacgc | |
| | agatataagtgagcccaaacgggtgcgcgagtcagttgcg | |
| | cagccatcgacgtcagacgcggaagcttcgatcaactacg | |
| | cagacaggtaccaaaacaaatgttctcgtcacgtgggcat | |
| | gaatctgatgctgtttccctgcagacaatgcgagagaatg | |
| | aatcagaattcaaatatctgcttcactcacggacagaaag | |
| | actgtttagagtgctttcccgtgtcagaatctcaacccgt | |
| | ttctgtcgtcaaaaaggcgtatcagaaactgtgctacatt | |
| | catcatatcatgggaaaggtgccagacgcttgcactgcct | |
| | gcgatctggtcaatgtggatttggatgactgcatctttga | |
| | acaataaatgacttaaaccaggtatggctgccgatggtta | |
| | tcttccagattggctcgaggacaaccttagtgaaggaatt | |
| | cgcgagtggtgggctttgaaacctggagcccctcaaccca | |
| | aggcaaatcaacaacatcaagacaacgctcgaggtcttgt | |
| | gcttccgggttacaaataccttggacccggcaacggactc | |
| | gacaagggggagccggtcaacgcagcagacgcggcggccc | |
| | tcgagcacgacaaggcctacgaccagcagctcaaggccgg | |
| | agacaacccgtacctcaagtacaaccacgccgacgccgag | |
| | ttccaggagcggctcaaagaagatacgtcttttgggggca | |
| | acctcgggcgagcagtcttccaggccaaaaagaggcttct | |
| | tgaacctcttggtctggttgaggaagcggctaagacggct | |
| | cctggaaagaagaggcctgtagagcagtctcctcaggaac | |
| | cggactcctccgcgggtattggcaaatcgggtgcacagcc | |
| | cgctaaaaagagactcaatttcggtcagactggcgacaca | |
| | gagtcagtcccagaccctcaaccaatcggagaacctcccg | |
| | cagccccctcaggtgtgggatctcttacaatggcttcagg | |
| | tggtggcgcaccagtggcagacaataacgaaggtgccgat | |
| | ggagtgggtagttcctcgggaaattggcattgcgattccc | |
| | aatggctgggggacagagtcatcaccaccagcacccgaac | |
| | ctgggccctgcccacctacaacaatcacctctacaagcaa | |
| | atctccaacagcacatctggaggatcttcaaatgacaacg | |
| | cctacttcggctacagcaccccctgggggtattttgactt | |
| | caacagattccactgccacttctcaccacgtgactggcag | |
| | cgactcatcaacaacaactggggattccggcctaagcgac | |
| | tcaacttcaagctcttcaacattcaggtcaaagaggttac | |
| | ggacaacaatggagtcaagaccatcgccaataaccttacc | |
| | agcacggtccaggtcttcacggactcagactatcagctcc | |
| | cgtacgtgctcgggtcggctcacgagggctgcctcccgcc | |
| | gttcccagcggacgttttcatgattcctcagtacgggtat | |
| | ctgacgcttaatgatggaagccaggccgtgggtcgttcgt | |
| | ccttttactgcctggaatatttcccgtcgcaaatgctaag | |
| | aacgggtaacaacttccagttcagctacgagtttgagaac | |
| | gtacctttccatagcagctacgctcacagccaaagcctgg | |
| | accgactaatgaatccactcatcgaccaatacttgtacta | |
| | tctctcaaagactattaacggttctggacagaatcaacaa | |
| | acgctaaaattcagtgtggccggacccagcaacatggctg | |
| | tccagggaagaaactacatacctggacccagctaccgaca | |
| | acaacgtgtctcaaccactgtgactcaaaacaacaacagc | |
| | gaatttgcttggcctggagcttcttcttgggctctcaatg | |
| | gacgtaatagcttgatgaatcctggacctgctatggccag | |
| | ccacaaagaaggagaggaccgtttctttcctttgtctgga | |
| | tctttaatttttggcaaacaaggaactggaagagacaacg | |
| | tggatgcggacaaagtcatgataaccaacgaagaagaaat | |
| | taaaactactaacccggtagcaacggagtcctatggacaa | |
| | gtggccacaaaccattgtagcactagcatcagggcgcaga | |
| | ccggctgggttcaaaaccaaggaatacttccgggtatggt | |
| | ttggcaggacagagatgtgtacctgcaaggacccatttgg | |
| | gccaaaattcctcacacggacggcaactttcacccttctc | |
| | cgctgatgggagggtttggaatgaagcacccgcctcctca | |
| | gatcctcatcaaaaacacacctgtacctgcggatcctcca | |
| | acggccttcaacaaggacaagctgaactctttcatcaccc | |
| | agtattctactggccaagtcagcgtggagatcgagtggga | |
| | gctgcagaaggaaaacagcaagcgctggaacccggagatc | |
| | cagtacacttccaactattacaagtctaataatgttgaat | |
| | ttgctgttaatactgaaggtgtatatagtgaaccccgccc | |
| | cattggcaccagatacctgactcgtaatctgtaattgctt | |
| | gttaatcaataaaccgtttaattcgtttcagttgaacttt | |
| | ggtctctgcgaaggcgcgccataacttcgttggcgtaatc | |
| | atggtcatagctgtttcctgtgtgaaattgttatccgctc | |
| | acaattccacacaacatacgagccggaagcataaagtgta | |
| | aagcctggggtgcctaatgagtgagctaactcacattaat | |
| | tgcgttgcgctcactgcccgctttccagtcgggaaacctg | |
| | tcgtgccagctgcattaatgaatcggccaacgcgcgggga | |
| | gaggcggtttgcgtattgggcgctcttccgcttcctcgct | |
| | cactgactcgctgcgctcggtcgttcggctgcggcgagcg | |
| | gtatcagctcactcaaaggcggtaatacggttatccacag | |
| | aatcaggggataacgcaggaaagaacatgtgagcaaaagg | |
| | ccagcaaaaggccaggaaccgtaaaaaggccgcgttgctg | |
| | gcgtttttccataggctccgcccccctgacgagcatcaca | |
| | aaaatcgacgctcaagtcagaggtggcgaaacccgacagg | |
| | actataaagataccaggcgtttccccctggaagctccctc | |
| | gtgcgctctcctgttccgaccctgccgcttaccggatacc | |
| | tgtccgcctttctcccttcgggaagcgtggcgctttctca | |
| | tagctcacgctgtaggtatctcagttcggtgtaggtcgtt | |
| | cgctccaagctgggctgtgtgcacgaaccccccgttcagc | |
| | ccgaccgctgcgccttatccggtaactatcgtcttgagtc | |
| | caacccggtaagacacgacttatcgccactggcagcagcc | |
| | actggtaacaggattagcagagcgaggtatgtaggcggtg | |
| | ctacagagttcttgaagtggtggcctaactacggctacac | |
| | tagaagaacagtatttggtatctgcgctctgctgaagcca | |
| | gttaccttcggaaaaagagttggtagctcttgatccggca | |
| | aacaaaccaccgctggtagcggtggtttttttgtttgcaa | |
| | gcagcagattacgcgcagaaaaaaaggatctcaagaagat | |
| | cctttgatcttttctacggggtctgacgctcagtggaacg | |
| | aaaactcacgttaagggattttggtcatgagattatcaaa | |
| | aaggatcttcacctagatccttttaaattaaaaatgaagt | |
| | tttaaatcaatctaaagtatatatgagtaaacttggtctg | |
| | acagttaccaatgcttaatcagtgaggcacctatctcagc | |
| | gatctgtctatttcgttcatccatagttgcctgactcccc | |
| | gtcgtgtagataactacgatacgggagggcttaccatctg | |
| | gccccagtgctgcaatgataccgcgagacccacgctcacc | |
| | ggctccagatttatcagcaataaaccagccagccggaagg | |
| | gccgagcgcagaagtggtcctgcaactttatccgcctcca | |
| | tccagtctattaattgttgccgggaagctagagtaagtag | |
| | ttcgccagttaatagtttgcgcaacgttgttgccattgct | |
| | acaggcatcgtggtgtcacgctcgtcgtttggtatggctt | |
| | cattcagctccggttcccaacgatcaaggcgagttacatg | |
| | atcccccatgttgtgcaaaaaagcggttagctccttcggt | |
| | cctccgatcgttgtcagaagtaagttggccgcagtgttat | |
| | cactcatggttatggcagcactgcataattctcttactgt | |
| | catgccatccgtaagatgcttttctgtgactggtgagtac | |
| | tcaaccaagtcattctgagaatagtgtatgcggcgaccga | |
| | gttgctcttgcccggcgtcaatacgggataataccgcgcc | |
| | acatagcagaactttaaaagtgctcatcattggaaaacgt | |
| | tcttcggggcgaaaactctcaaggatcttaccgctgttga | |
| | gatccagttcgatgtaacccactcgtgcacccaactgatc | |
| | ttcagcatcttttactttcaccagcgtttctgggtgagca | |
| | aaaacaggaaggcaaaatgccgcaaaaaagggaataaggg | |
| | cgacacggaaatgttgaatactcatactcttcctttttca | |
| | atattattgaagcatttatcagggttattgtctcatgagc | |
| | ggatacatatttgaatgtatttagaaaaataaacaaatag | |
| | gggttccgcgcacatttccccgaaaagtgccacctgacgt | |
| | ctaagaaaccattattatcatgacattaacctataaaaat | |
| | aggcgtatcacgaggccctttcgtc | |
| A rep/cap | tcgcgcgtttcggtgatgacggtgaaaacctctgacacat | 9 |
| plasmid with | gcagctcccggagacggtcacagcttgtctgtaagcggat | |
| modified AAV9 | gccgggagcagacaagcccgtcagggcgcgtcagcgggtg | |
| cap: | ttggcgggtgtcggggctggcttaactatgcggcatcaga | |
| pZC96_REP2- | gcagattgtactgagagtgcaccatatgcggtgtgaaata | |
| CR9-10 | ccgcacagatgcgtaaggagaaaataccgcatcaggcgcc | |
| | attcgccattcaggctgcgcaactgttgggaagggcgatc | |
| | ggtgcgggcctcttcgctattacgccagctggcgaaaggg | |
| | ggatgtgctgcaaggcgattaagttgggtaacgccagggt | |
| | tttcccagtcacgacgttgtaaaacgacggccagtgaatt | |
| | gacgcgtattgggatgtagttaatgattaacccgccatgc | |
| | tacttatctacgtagccatgctctaggaagatcggaattc | |
| | gccctattagagtcctgtattagaggtcacgtgagtgttt | |
| | tgcgacattttgcgacaccatgtggtcacgctgggtattt | |
| | aagcccgagtgagcacgcagggtctccattttgaagcggg | |
| | aggtttgaacgcgcagccaccacggcggggttttacgaga | |
| | ttgtgattaaggtccccagcgaccttgacgggcatctgcc | |
| | cggcatttctgacagctttgtgaactgggtggccgagaag | |
| | gaatgggagttgccgccagattctgacatggatctgaatc | |
| | tgattgagcaggcacccctgaccgtggccgagaagctgca | |
| | gcgcgactttctgacggaatggcgccgtgtgagtaaggcc | |
| | ccggaggcccttttctttgtgcaatttgagaagggagaga | |
| | gctacttccacatgcacgtgctcgtggaaaccaccggggt | |
| | gaaatccatggttttgggacgtttcctgagtcagattcgc | |
| | gaaaaactgattcagagaatttaccgcgggatcgagccga | |
| | ctttgccaaactggttcgcggtcacaaagaccagaaatgg | |
| | cgccggaggcgggaacaaggtggtggatgagtgctacatc | |
| | cccaattacttgctccccaaaacccagcctgagctccagt | |
| | gggcgtggactaatatggaacagtatttaagcgcctgttt | |
| | gaatctcacggagcgtaaacggttggtggcgcagcatctg | |
| | acgcacgtgtcgcagacgcaggagcagaacaaagagaatc | |
| | agaatcccaattctgatgcgccggtgatcagatcaaaaac | |
| | ttcagccaggtacatggagctggtcgggtggctcgtggac | |
| | aaggggattacctcggagaagcagtggatccaggaggacc | |
| | aggcctcatacatctccttcaatgcggcctccaactcgcg | |
| | gtcccaaatcaaggctgccttggacaatgcgggaaagatt | |
| | atgagcctgactaaaaccgcccccgactacctggtgggcc | |
| | agcagcccgtggaggacatttccagcaatcggatttataa | |
| | aattttggaactaaacgggtacgatccccaatatgcggct | |
| | tccgtctttctgggatgggccacgaaaaagttcggcaaga | |
| | ggaacaccatctggctgtttgggcctgcaactaccgggaa | |
| | gaccaacatcgcggaggccatagcccacactgtgcccttc | |
| | tacgggtgcgtaaactggaccaatgagaactttcccttca | |
| | acgactgtgtcgacaagatggtgatctggtgggaggaggg | |
| | gaagatgaccgccaaggtcgtggagtcggccaaagccatt | |
| | ctcggaggaagcaaggtgcgcgtggaccagaaatgcaagt | |
| | cctcggcccagatagacccgactcccgtgatcgtcacctc | |
| | caacaccaacatgtgcgccgtgattgacgggaactcaacg | |
| | accttcgaacaccagcagccgttgcaagaccggatgttca | |
| | aatttgaactcacccgccgtctggatcatgactttgggaa | |
| | ggtcaccaagcaggaagtcaaagactttttccggtgggca | |
| | aaggatcacgtggttgaggtggagcatgaattctacgtca | |
| | aaaagggtggagccaagaaaagacccgcccccagtgacgc | |
| | agatataagtgagcccaaacgggtgcgcgagtcagttgcg | |
| | cagccatcgacgtcagacgcggaagcttcgatcaactacg | |
| | cagacaggtaccaaaacaaatgttctcgtcacgtgggcat | |
| | gaatctgatgctgtttccctgcagacaatgcgagagaatg | |
| | aatcagaattcaaatatctgcttcactcacggacagaaag | |
| | actgtttagagtgctttcccgtgtcagaatctcaacccgt | |
| | ttctgtcgtcaaaaaggcgtatcagaaactgtgctacatt | |
| | catcatatcatgggaaaggtgccagacgcttgcactgcct | |
| | gcgatctggtcaatgtggatttggatgactgcatctttga | |
| | acaataaatgacttaaaccaggtatggctgccgatggtta | |
| | tcttccagattggctcgaggacaaccttagtgaaggaatt | |
| | cgcgagtggtgggctttgaaacctggagcccctcaaccca | |
| | aggcaaatcaacaacatcaagacaacgctcgaggtcttgt | |
| | gcttccgggttacaaataccttggacccggcaacggactc | |
| | gacaagggggagccggtcaacgcagcagacgcggcggccc | |
| | tcgagcacgacaaggcctacgaccagcagctcaaggccgg | |
| | agacaacccgtacctcaagtacaaccacgccgacgccgag | |
| | ttccaggagcggctcaaagaagatacgtcttttgggggca | |
| | acctcgggcgagcagtcttccaggccaaaaagaggcttct | |
| | tgaacctcttggtctggttgaggaagcggctaagacggct | |
| | cctggaaagaagaggcctgtagagcagtctcctcaggaac | |
| | cggactcctccgcgggtattggcaaatcgggtgcacagcc | |
| | cgctaaaaagagactcaatttcggtcagactggcgacaca | |
| | gagtcagtcccagaccctcaaccaatcggagaacctcccg | |
| | cagccccctcaggtgtgggatctcttacaatggcttcagg | |
| | tggtggcgcaccagtggcagacaataacgaaggtgccgat | |
| | ggagtgggtagttcctcgggaaattggcattgcgattccc | |
| | aatggctgggggacagagtcatcaccaccagcacccgaac | |
| | ctgggccctgcccacctacaacaatcacctctacaagcaa | |
| | atctccaacagcacatctggaggatcttcaaatgacaacg | |
| | cctacttcggctacagcaccccctgggggtattttgactt | |
| | caacagattccactgccacttctcaccacgtgactggcag | |
| | cgactcatcaacaacaactggggattccggcctaagcgac | |
| | tcaacttcaagctcttcaacattcaggtcaaagaggttac | |
| | ggacaacaatggagtcaagaccatcgccaataaccttacc | |
| | agcacggtccaggtcttcacggactcagactatcagctcc | |
| | cgtacgtgctcgggtcggctcacgagggctgcctcccgcc | |
| | gttcccagcggacgttttcatgattcctcagtacgggtat | |
| | ctgacgcttaatgatggaagccaggccgtgggtcgttcgt | |
| | ccttttactgcctggaatatttcccgtcgcaaatgctaag | |
| | aacgggtaacaacttccagttcagctacgagtttgagaac | |
| | gtacctttccatagcagctacgctcacagccaaagcctgg | |
| | accgactaatgaatccactcatcgaccaatacttgtacta | |
| | tctctcaaagactattaacggttctggacagaatcaacaa | |
| | acgctaaaattcagtgtggccggacccagcaacatggctg | |
| | tccagggaagaaactacatacctggacccagctaccgaca | |
| | acaacgtgtctcaaccactgtgactcaaaacaacaacagc | |
| | gaatttgcttggcctggagcttcttcttgggctctcaatg | |
| | gacgtaatagcttgatgaatcctggacctgctatggccag | |
| | ccacaaagaaggagaggaccgtttctttcctttgtctgga | |
| | tctttaatttttggcaaacaaggaactggaagagacaacg | |
| | tggatgcggacaaagtcatgataaccaacgaagaagaaat | |
| | taaaactactaacccggtagcaacggagtcctatggacaa | |
| | gtggccacaaaccaccaggctaattatggtcaggcgcaga | |
| | ccggctgggttcaaaaccaaggaatacttccgggtatggt | |
| | ttggcaggacagagatgtgtacctgcaaggacccatttgg | |
| | gccaaaattcctcacacggacggcaactttcacccttctc | |
| | cgctgatgggagggtttggaatgaagcacccgcctcctca | |
| | gatcctcatcaaaaacacacctgtacctgcggatcctcca | |
| | acggccttcaacaaggacaagctgaactctttcatcaccc | |
| | agtattctactggccaagtcagcgtggagatcgagtggga | |
| | gctgcagaaggaaaacagcaagcgctggaacccggagatc | |
| | cagtacacttccaactattacaagtctaataatgttgaat | |
| | ttgctgttaatactgaaggtgtatatagtgaaccccgccc | |
| | cattggcaccagatacctgactcgtaatctgtaattgctt | |
| | gttaatcaataaaccgtttaattcgtttcagttgaacttt | |
| | ggtctctgcgaaggcgcgccataacttcgttggcgtaatc | |
| | atggtcatagctgtttcctgtgtgaaattgttatccgctc | |
| | acaattccacacaacatacgagccggaagcataaagtgta | |
| | aagcctggggtgcctaatgagtgagctaactcacattaat | |
| | tgcgttgcgctcactgcccgctttccagtcgggaaacctg | |
| | tcgtgccagctgcattaatgaatcggccaacgcgcgggga | |
| | gaggcggtttgcgtattgggcgctcttccgcttcctcgct | |
| | cactgactcgctgcgctcggtcgttcggctgcggcgagcg | |
| | gtatcagctcactcaaaggcggtaatacggttatccacag | |
| | aatcaggggataacgcaggaaagaacatgtgagcaaaagg | |
| | ccagcaaaaggccaggaaccgtaaaaaggccgcgttgctg | |
| | gcgtttttccataggctccgcccccctgacgagcatcaca | |
| | aaaatcgacgctcaagtcagaggtggcgaaacccgacagg | |
| | actataaagataccaggcgtttccccctggaagctccctc | |
| | gtgcgctctcctgttccgaccctgccgcttaccggatacc | |
| | tgtccgcctttctcccttcgggaagcgtggcgctttctca | |
| | tagctcacgctgtaggtatctcagttcggtgtaggtcgtt | |
| | cgctccaagctgggctgtgtgcacgaaccccccgttcagc | |
| | ccgaccgctgcgccttatccggtaactatcgtcttgagtc | |
| | caacccggtaagacacgacttatcgccactggcagcagcc | |
| | actggtaacaggattagcagagcgaggtatgtaggcggtg | |
| | ctacagagttcttgaagtggtggcctaactacggctacac | |
| | tagaagaacagtatttggtatctgcgctctgctgaagcca | |
| | gttaccttcggaaaaagagttggtagctcttgatccggca | |
| | aacaaaccaccgctggtagcggtggtttttttgtttgcaa | |
| | gcagcagattacgcgcagaaaaaaaggatctcaagaagat | |
| | cctttgatcttttctacggggtctgacgctcagtggaacg | |
| | aaaactcacgttaagggattttggtcatgagattatcaaa | |
| | aaggatcttcacctagatccttttaaattaaaaatgaagt | |
| | tttaaatcaatctaaagtatatatgagtaaacttggtctg | |
| | acagttaccaatgcttaatcagtgaggcacctatctcagc | |
| | gatctgtctatttcgttcatccatagttgcctgactcccc | |
| | gtcgtgtagataactacgatacgggagggcttaccatctg | |
| | gccccagtgctgcaatgataccgcgagacccacgctcacc | |
| | ggctccagatttatcagcaataaaccagccagccggaagg | |
| | gccgagcgcagaagtggtcctgcaactttatccgcctcca | |
| | tccagtctattaattgttgccgggaagctagagtaagtag | |
| | ttcgccagttaatagtttgcgcaacgttgttgccattgct | |
| | acaggcatcgtggtgtcacgctcgtcgtttggtatggctt | |
| | cattcagctccggttcccaacgatcaaggcgagttacatg | |
| | atcccccatgttgtgcaaaaaagcggttagctccttcggt | |
| | cctccgatcgttgtcagaagtaagttggccgcagtgttat | |
| | cactcatggttatggcagcactgcataattctcttactgt | |
| | catgccatccgtaagatgcttttctgtgactggtgagtac | |
| | tcaaccaagtcattctgagaatagtgtatgcggcgaccga | |
| | gttgctcttgcccggcgtcaatacgggataataccgcgcc | |
| | acatagcagaactttaaaagtgctcatcattggaaaacgt | |
| | tcttcggggcgaaaactctcaaggatcttaccgctgttga | |
| | gatccagttcgatgtaacccactcgtgcacccaactgatc | |
| | ttcagcatcttttactttcaccagcgtttctgggtgagca | |
| | aaaacaggaaggcaaaatgccgcaaaaaagggaataaggg | |
| | cgacacggaaatgttgaatactcatactcttcctttttca | |
| | atattattgaagcatttatcagggttattgtctcatgagc | |
| | ggatacatatttgaatgtatttagaaaaataaacaaatag | |
| | gggttccgcgcacatttccccgaaaagtgccacctgacgt | |
| | ctaagaaaccattattatcatgacattaacctataaaaat | |
| | aggcgtatcacgaggccctttcgtc | |
| REP protein | TAGFYEIVIKVPSDLDEHLPGISDSFVNWVAEKEWELPPD | 10 |
| | SDMDLNLIEQAPLTVAEKLQRDFLTEWRRVSKAPEALFFV | |
| | QFEKGESYFHMHVLVETTGVKSMVLGRFLSQIREKLIQRI | |
| | YRGIEPTLPNWFAVTKTRNGAGGGNKVVDECYIPNYLLPK | |
| | TQPELQWAWTNMEQYLSACLNLTERKRLVAQHLTHVSQTQ | |
| | EQNKENQNPNSDAPVIRSKTSARYMELVGWLVDKGITSEK | |
| | QWIQEDQASYISFNAASNSRSQIKAALDNAGKIMSLIKTA | |
| | PDYLVGQQPVEDISSNRIYKILELNGYDPQYAASVFLGWA | |
| | TKKFGKRNTIWLFGPATTGKTNIAEAIAHTVPFYGCVNWT | |
| | NENFPFNDCVDKMVIWWEEGKMTAKVVESAKAILGGSKVR | |
| | VDQKCKSSAQIDPTPVIVTSNTNMCAVIDGNSTTFEHQQP | |
| | LQDRMFKFELTRRLDHDFGKVTKQEVKDFFRWAKDHVVEV | |
| | EHEFYVKKGGAKKRPAPSDADISEPKRVRESVAQPSTSDA | |
| | EASINYADRYQNKCSRHVGMNLMLFPCRQCERMNQNSNIC | |
| | FTHGQKDCLECFPVSESQPVSVVKKAYQKLCYIHHIMGKV | |
| | PDACTACDLVNVDLDDCIFEQ | |
| AAV9 VP1 | MAADGYLPDWLEDNLSEGIREWWALKPGAPQPKANQQHQD | 11 |
| protein | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVATNHQSAQAQAQTGWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| AAV9 VP2 | TAPGKKRPVEQSPQEPDSSAGIGKSGAQPAKKRLNFGQTG | 12 |
| protein | DTESVPDPQPIGEPPAAPSGVGSLTMASGGGAPVADNNEG | |
| | ADGVGSSSGNWHCDSQWLGDRVITTSTRTWALPTYNNHLY | |
| | KQISNSTSGGSSNDNAYFGYSTPWGYFDFNRFHCHFSPRD | |
| | WQRLINNNWGFRPKRLNFKLFNIQVKEVTDNNGVKTIANN | |
| | LTSTVQVFTDSDYQLPYVLGSAHEGCLPPFPADVFMIPQY | |
| | GYLTLNDGSQAVGRSSFYCLEYFPSQMLRTGNNFQFSYEF | |
| | ENVPFHSSYAHSQSLDRLMNPLIDQYLYYLSKTINGSGQN | |
| | QQTLKFSVAGPSNMAVQGRNYIPGPSYRQQRVSTTVTQNN | |
| | NSEFAWPGASSWALNGRNSLMNPGPAMASHKEGEDRFFPL | |
| | SGSLIFGKQGTGRDNVDADKVMITNEEEIKTTNPVATESY | |
| | GQVATNHQSAQAQAQTGWVQNQGILPGMVWQDRDVYLQGP | |
| | IWAKIPHTDGNFHPSPLMGGFGMKHPPPQILIKNTPVPAD | |
| | PPTAFNKDKLNSFITQYSTGQVSVEIEWELQKENSKRWNP | |
| | EIQYTSNYYKSNNVEFAVNTEGVYSEPRPIGTRYLTRNL | |
| AAV9 VP3 | MASGGGAPVADNNEGADGVGSSSGNWHCDSQWLGDRVITT | 13 |
| protein | STRTWALPTYNNHLYKQISNSTSGGSSNDNAYFGYSTPWG | |
| | YFDFNRFHCHFSPRDWQRLINNNWGFRPKRLNFKLFNIQV | |
| | KEVTDNNGVKTIANNLTSTVQVFTDSDYQLPYVLGSAHEG | |
| | CLPPFPADVFMIPQYGYLTLNDGSQAVGRSSFYCLEYFPS | |
| | QMLRTGNNFQFSYEFENVPFHSSYAHSQSLDRLMNPLIDQ | |
| | YLYYLSKTINGSGQNQQTLKFSVAGPSNMAVQGRNYIPGP | |
| | SYRQQRVSTTVTQNNNSEFAWPGASSWALNGRNSLMNPGP | |
| | AMASHKEGEDRFFPLSGSLIFGKQGTGRDNVDADKVMITN | |
| | EEEIKTTNPVATESYGQVATNHQSAQAQAQTGWVQNQGIL | |
| | PGMVWQDRDVYLQGPIWAKIPHTDGNFHPSPLMGGFGMKH | |
| | PPPQILIKNTPVPADPPTAFNKDKLNSFITQYSTGQVSVE | |
| | IEWELQKENSKRWNPEIQYTSNYYKSNNVEFAVNTEGVYS | |
| | EPRPIGTRYLTRNL | |
| AAP9 assembly | LATQSQSQTLNQSENLPQPPQVWDLLQWLQVVAHQWQTIT | 14 |
| activating | KVPMEWVVPREIGIAIPNGWGTESSPPAPEPGPCPPTTIT | |
| protein | STSKSPTAHLEDLQMTTPTSATAPPGGILTSTDSTATSHH | |
| | VTGSDSSTTTGDSGLSDSTSSSSTFRSKRLRTTMESRPSP | |
| | ITLPARSRSSRTQTISSRTCSGRLTRAASRRSQRTFS | |
| AAV9 VR-IV | NGSGQNQ | 15 |
| AAV9 VR-V | NNSEFA | 16 |
| AAV9 VR-VII | GRDNV | 17 |
| AAV9 VR-VIII | ATNHQSAQAQAQTG | 18 |
| AAV5 VP1 | MSFVDHPPDWLEEVGEGLREFLGLEAGPPKPKPNQQHQDQ | 19 |
| protein | ARGLVLPGYNYLGPGNGLDRGEPVNRADEVAREHDISYNE | |
| | QLEAGDNPYLKYNHADAEFQEKLADDTSFGGNLGKAVFQA | |
| | KKRVLEPFGLVEEGAKTAPTGKRIDDHFPKRKKARTEEDS | |
| | KPSTSSDAEAGPSGSQQLQIPAQPASSLGADTMSAGGGGP | |
| | LGDNNQGADGVGNASGDWHCDSTWMGDRVVTKSTRTWVLP | |
| | SYNNHQYREIKSGSVDGSNANAYFGYSTPWGYFDFNRFHS | |
| | HWSPRDWQRLINNYWGFRPRSLRVKIFNIQVKEVTVQDST | |
| | TTIANNLTSTVQVFTDDDYQLPYVVGNGTEGCLPAFPPQV | |
| | FTLPQYGYATLNRDNTENPTERSSFFCLEYFPSKMLRTGN | |
| | NFEFTYNFEEVPFHSSFAPSQNLFKLANPLVDQYLYRFVS | |
| | TNNTGGVQFNKNLAGRYANTYKNWFPGPMGRTQGWNLGSG | |
| | VNRASVSAFATTNRMELEGASYQVPPQPNGMTNNLQGSNT | |
| | YALENTMIFNSQPANPGTTATYLEGNMLITSESETQPVNR | |
| | VAYNVGGQMATNNQSSTTAPATGTYNLQEIVPGSVWMERD | |
| | VYLQGPIWAKIPETGAHFHPSPAMGGFGLKHPPPMMLIKN | |
| | TPVPGNITSFSDVPVSSFITQYSTGQVTVEMEWELKKENS | |
| | KRWNPEIQYTNNYNDPQFVDFAPDSTGEYRTTRPIGTRYL | |
| | TRPL | |
| AAV5 VP2 | TAPTGKRIDDHFPKRKKARTEEDSKPSTSSDAEAGPSGSQ | 20 |
| protein | QLQIPAQPASSLGADTMSAGGGGPLGDNNQGADGVGNASG | |
| | DWHCDSTWMGDRVVTKSTRTWVLPSYNNHQYREIKSGSVD | |
| | GSNANAYFGYSTPWGYFDFNRFHSHWSPRDWQRLINNYWG | |
| | FRPRSLRVKIFNIQVKEVTVQDSTTTIANNLTSTVQVFTD | |
| | DDYQLPYVVGNGTEGCLPAFPPQVFTLPQYGYATLNRDNT | |
| | ENPTERSSFFCLEYFPSKMLRTGNNFEFTYNFEEVPFHSS | |
| | FAPSQNLFKLANPLVDQYLYRFVSTNNTGGVQFNKNLAGR | |
| | YANTYKNWFPGPMGRTQGWNLGSGVNRASVSAFATTNRME | |
| | LEGASYQVPPQPNGMTNNLQGSNTYALENTMIFNSQPANP | |
| | GTTATYLEGNMLITSESETQPVNRVAYNVGGQMATNNQSS | |
| | TTAPATGTYNLQEIVPGSVWMERDVYLQGPIWAKIPETGA | |
| | HFHPSPAMGGFGLKHPPPMMLIKNTPVPGNITSFSDVPVS | |
| | SFITQYSTGQVTVEMEWELKKENSKRWNPEIQYTNNYNDP | |
| | QFVDFAPDSTGEYRTTRPIGTRYLTRPL | |
| AAV5 VP3 | MSAGGGGPLGDNNQGADGVGNASGDWHCDSTWMGDRVVTK | 21 |
| protein | STRTWVLPSYNNHQYREIKSGSVDGSNANAYFGYSTPWGY | |
| | FDFNRFHSHWSPRDWQRLINNYWGFRPRSLRVKIFNIQVK | |
| | EVTVQDSTTTIANNLTSTVQVFTDDDYQLPYVVGNGTEGC | |
| | LPAFPPQVFTLPQYGYATLNRDNTENPTERSSFFCLEYFP | |
| | SKMLRTGNNFEFTYNFEEVPFHSSFAPSQNLFKLANPLVD | |
| | QYLYRFVSTNNTGGVQFNKNLAGRYANTYKNWFPGPMGRT | |
| | QGWNLGSGVNRASVSAFATTNRMELEGASYQVPPQPNGMT | |
| | NNLQGSNTYALENTMIFNSQPANPGTTATYLEGNMLITSE | |
| | SETQPVNRVAYNVGGQMATNNQSSTTAPATGTYNLQEIVP | |
| | GSVWMERDVYLQGPIWAKIPETGAHFHPSPAMGGFGLKHP | |
| | PPMMLIKNTPVPGNITSFSDVPVSSFITQYSTGQVTVEME | |
| | WELKKENSKRWNPEIQYTNNYNDPQFVDFAPDSTGEYRTT | |
| | RPIGTRYLTRPL | |
| AAP5 assembly | LDPADPSSCKSQPNQPQVWELIQCLREVAAHWATITKVPM | 22 |
| activating | EWAMPREIGIAIPRGWGTESSPSPPEPGCCPATTTTSTER | |
| protein | SKAAPSTEATPTPTLDTAPPGGTLTLTASTATGAPETGKD | |
| | SSTTTGASDPGPSESKSSTFKSKRSRCRTPPPPSPTTSPP | |
| | PSKCLRTTTTSCPTSSATGPRDACRPSLRRSLRCRSTVTR | |
| | R | |
| ZC375 capsid | MAADGYLPDWLEDNLSEGIREWWALKPGAPQPKANQQHQD | 23 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTIKGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVATNHNSTYLGAQTGWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC401 capsid | MAADGYLPDWLEDNLSEGIREWWALKPGAPQPKANQQHQD | 24 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVATNHMMTTARAQTGWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC428 capsid | MAADGYLPDWLEDNLSEGIREWWALKPGAPQPKANQQHQD | 25 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVATNHNSTYLGAQTGWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC478 capsid | MAADGYLPDWLEDNLSEGIREWWALKPGAPQPKANQQHQD | 26 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVATNHCSTSIRAQTGWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC96 capsid | MAADGYLPDWLEDNLSEGIREWWALKPGAPQPKANQQHQD | 44 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVATNHQANYGQAQTGWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC621 | MAADGYLPDWLEDNLSEGIREWWALKPGAPQPKANQQHQD | 93 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVADVQQLPGFQAQTEWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC623 | MAADGYLPDWLEDNLSEGIREWWALKPGAPQPKANQQHQD | 94 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVADVQQHPGFQAQTEWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC625 | MAADGYLPDWLEDNLSEGIREWWALKPGAPQPKANQQHQD | 95 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSHVRGDLSGQNQQTLKFSVAGPSNMAV | |
| | QGRNYIPGPSYRQQRVSTTVTQNNNSEFAWPGASSWALNG | |
| | RNSLMNPGPAMASHKEGEDRFFPLSGSLIFGKQGTGRDNV | |
| | DADKVMITNEEEIKTTNPVATESYGQVATNHQSAQAQAQT | |
| | GWVQNQGILPGMVWQDRDVYLQGPIWAKIPHTDGNFHPSP | |
| | LMGGFGMKHPPPQILIKNTPVPADPPTAFNKDKLNSFITQ | |
| | YSTGQVSVEIEWELQKENSKRWNPEIQYTSNYYKSNNVEF | |
| | AVNTEGVYSEPRPIGTRYLTRNL | |
| ZC626 | MAADGYLPDWLEDNLSEGIREWWALKPGAPQPKANQQHQD | 96 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGVVSSGARSGQNQQTLKFSVAGPSNMAV | |
| | QGRNYIPGPSYRQQRVSTTVTQNNNSEFAWPGASSWALNG | |
| | RNSLMNPGPAMASHKEGEDRFFPLSGSLIFGKQGTGRDNV | |
| | DADKVMITNEEEIKTTNPVATESYGQVATNHQSAQAQAQT | |
| | GWVQNQGILPGMVWQDRDVYLQGPIWAKIPHTDGNFHPSP | |
| | LMGGFGMKHPPPQILIKNTPVPADPPTAFNKDKLNSFITQ | |
| | YSTGQVSVEIEWELQKENSKRWNPEIQYTSNYYKSNNVEF | |
| | AVNTEGVYSEPRPIGTRYLTRNL | |
| ZC630 | MAADGYLPDWLEDNLSEGIREWWALKPGAPQPKANQQHQD | 97 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVATNHQSSVRGDAARLAGAQT | |
| | GWVQNQGILPGMVWQDRDVYLQGPIWAKIPHTDGNFHPSP | |
| | LMGGFGMKHPPPQILIKNTPVPADPPTAFNKDKLNSFITQ | |
| | YSTGQVSVEIEWELQKENSKRWNPEIQYTSNYYKSNNVEF | |
| | AVNTEGVYSEPRPIGTRYLTRNL | |
| ZC631 | MAADGYLPDWLEDNLSEGIREWWALKPGAPQPKANQQHQD | 98 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVADVQQVPGFQAQTMWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC632 | MAADGYLPDWLEDNLSEGIREWWALKPGAPQPKANQQHQD | 99 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVADVQQVPGFQAQTQWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC633 | MAADGYLPDWLEDNLSEGIREWWALKPGAPQPKANQQHQD | 100 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVADVQQLPGFGAQTEWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC634 | MAADGYLPDWLEDNLSEGIREWWALKPGAPQPKANQQHQD | 101 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVADVQQLPGFRPETEWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |
| ZC635 | MAADGYLPDWLEDNLSEGIREWWALKPGAPQPKANQQHQD | 102 |
| | NARGLVLPGYKYLGPGNGLDKGEPVNAADAAALEHDKAYD | |
| | QQLKAGDNPYLKYNHADAEFQERLKEDTSFGGNLGRAVFQ | |
| | AKKRLLEPLGLVEEAAKTAPGKKRPVEQSPQEPDSSAGIG | |
| | KSGAQPAKKRLNFGQTGDTESVPDPQPIGEPPAAPSGVGS | |
| | LTMASGGGAPVADNNEGADGVGSSSGNWHCDSQWLGDRVI | |
| | TTSTRTWALPTYNNHLYKQISNSTSGGSSNDNAYFGYSTP | |
| | WGYFDFNRFHCHFSPRDWQRLINNNWGFRPKRLNFKLFNI | |
| | QVKEVTDNNGVKTIANNLTSTVQVFTDSDYQLPYVLGSAH | |
| | EGCLPPFPADVFMIPQYGYLTLNDGSQAVGRSSFYCLEYF | |
| | PSQMLRTGNNFQFSYEFENVPFHSSYAHSQSLDRLMNPLI | |
| | DQYLYYLSKTINGSGQNQQTLKFSVAGPSNMAVQGRNYIP | |
| | GPSYRQQRVSTTVTQNNNSEFAWPGASSWALNGRNSLMNP | |
| | GPAMASHKEGEDRFFPLSGSLIFGKQGTGRDNVDADKVMI | |
| | TNEEEIKTTNPVATESYGQVADVQQLPGFNAQTEWVQNQG | |
| | ILPGMVWQDRDVYLQGPIWAKIPHTDGNFHPSPLMGGFGM | |
| | KHPPPQILIKNTPVPADPPTAFNKDKLNSFITQYSTGQVS | |
| | VEIEWELQKENSKRWNPEIQYTSNYYKSNNVEFAVNTEGV | |
| | YSEPRPIGTRYLTRNL | |

### INCORPORATION BY REFERENCE

Various references such as patents, patent applications, and publications are cited herein, the disclosures of which are hereby incorporated herein by reference in their entireties. Also, all references mentioned herein are specifically incorporated by reference to disclose and describe the methods and/or materials in connection with which the publications are cited.

1. A method of producing viral particles and/or increasing viral particle titer, comprising:
transfecting or transducing cells in a cell culture with viral particles or a recombinant viral vector to be packaged in the cells into viral particles;
adding to the cell culture a selective HDAC6 inhibitor; and
culturing the cells in the cell culture.

2. The method of claim 1, wherein the cells are mammalian cells.

3. (canceled)

4. The method of claim 2, wherein the mammalian cells are HEK293 or HEK293T cells or cell line.

5. (canceled)

6. The method of claim 2, wherein the virus or recombinant viral vector is an AAV virus.

7-9. (canceled)

10. The method of claim 1, wherein the selective HDAC6 inhibitor is fluoroalkyl-oxadiazole derivative.

11. The method of claim 1, wherein the selective HDAC6 inhibitor is a compound Formula (I): 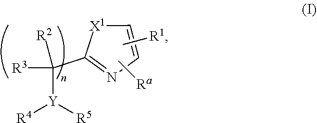
or a salt thereof,
wherein:
R¹ is selected from the group consisting of: 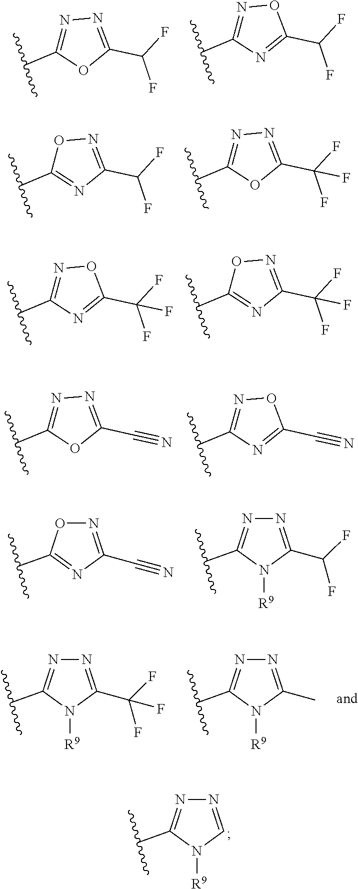
R^{a} is selected from the group consisting of H, halo, C₁₋₃ alkyl, cycloalkyl, haloalkyl, and alkoxy;
R² and R³ are independently selected from the group consisting of H, halogen, alkoxy, haloalkyl, aryl, heteroaryl, alkyl, and cycloalkyl each of which is optionally substituted, or R² and R³ together with the atom to which they are attached form a cycloalkyl or heterocyclyl;
R⁴ and R⁵ are independently selected from the group consisting of H, —(SO₂)R², —(SO₂)NR²R³, —(CO)R², —(CONR²R³), aryl, arylheteroaryl, alkylenearyl, heteroaryl, cycloalkyl, heterocyclyl, alkyl, haloalkyl, and alkoxy, each of which is optionally substituted, or R⁴ and R⁵ together with the atom to which they are attached form a cycloalkyl or heterocyclyl, each of which is optionally substituted;
R⁹ is selected from the group consisting of H, C₁-C₆ alkyl, haloalkyl, cycloalkyl and heterocyclyl;
X¹ is selected from the group consisting of S, O, NH and NR⁶, wherein R⁶ is selected from the group consisting of C₁-C₆ alkyl, alkoxy, haloalkyl, cycloalkyl and heterocyclyl;
Y is selected from the group consisting of CR², O, N, S, SO, and SO₂, wherein when Y is O, S, SO, or SO₂, R⁵ is not present and when R⁴ and R⁵ together with the atom to which they are attached form a cycloalkyl or heterocyclyl, Y is CR² or N; and
n is selected from 0, 1, and 2.

12-15. (canceled)

16. The method of claim 11, wherein the selective HDAC6 inhibitor is a compound having the formula: 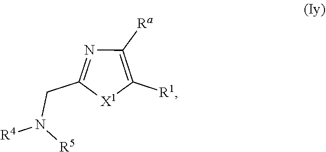
or a salt thereof,
wherein:
X¹ is S;
R^{a} is selected from the group consisting of H, halogen, and C₁₋₃ alkyl;
R¹ is 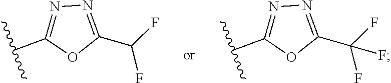 [SMILES: *c1nnc(C(F)F)o1 ; *c1nnc(C(F)(F)F)o1]
R² is selected from the group consisting of alkyl, alkoxy, and cycloalkyl, each of which is optionally substituted;
R³ is H or alkyl;
R⁴ is selected from the group consisting of alkyl, —(SO₂)R², —(SO₂)NR²R³, and —(CO)R²; and
R⁵ is aryl or heteroaryl; or R⁴ and R⁵ together with the atom to which they are attached form a heterocyclyl, each of which is optionally substituted.

17. The method of claim 16, wherein R^{a} is H.

18. The method of claim 16, wherein R is

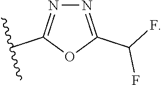 [SMILES: *c1nnc(C(F)F)o1]

19. The method of claim 16, wherein R⁴ is —(SO₂)R².

20. The method of claim 19, wherein —(SO₂)R² is —(SO₂)alkyl, —(SO₂)alkyleneheterocyclyl, —(SO₂)haloalkyl, —(SO₂)haloalkoxy, or —(SO₂)cycloalkyl.

21. The method of claim 16, wherein R⁵ is a 5- or 6-membered heteroaryl.

22. (canceled)

23. The method of claim 21, wherein the 5- to 6-membered heteroaryl is selected from the group consisting of 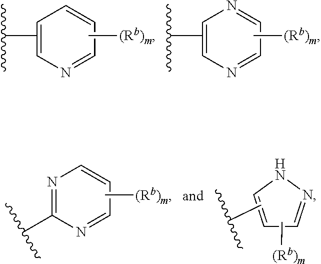
wherein R^{b} is halogen, alkyl, alkoxy, cycloalkyl, —CN, haloalkyl, or haloalkoxy; and m is 0 or 1.

24. The method of claim 23, wherein R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

25-29. (canceled)

30. The method of claim 27, wherein the selective HDAC6 inhibitor is a compound of Formula (Ik-1) or Formula (Ik-2): 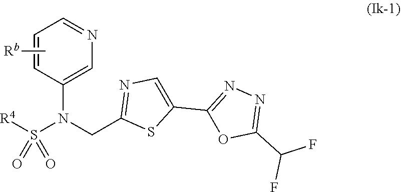
or a salt thereof,
wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

31. The method of claim 30, wherein R^{b} is H, halogen, haloalkyl, or haloalkoxy.

32. The method of claim 30, wherein R⁴ is optionally substituted alkyl or cycloalkyl.

33. The method of claim 30, wherein R⁴ is alkyl.

34-36. (canceled)

37. The method of claim 16, wherein the selective HDAC6 inhibitor is a compound of Formula: 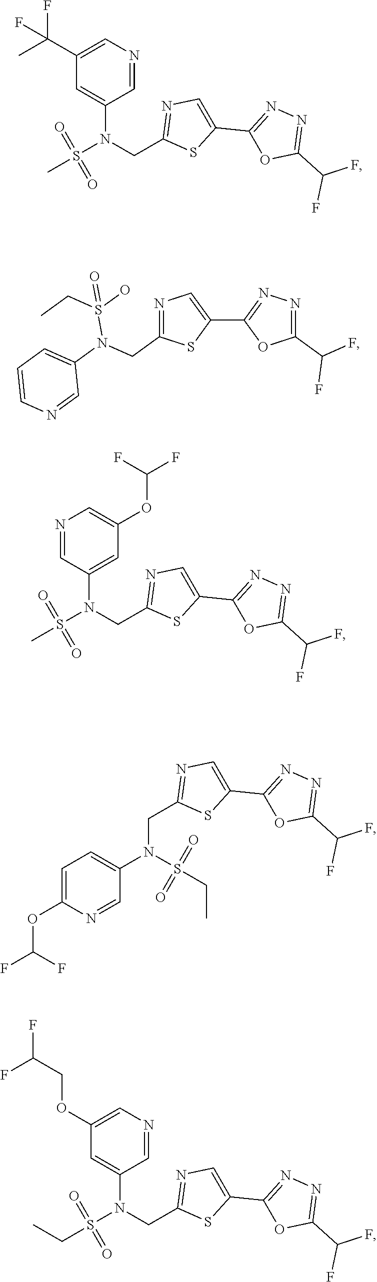 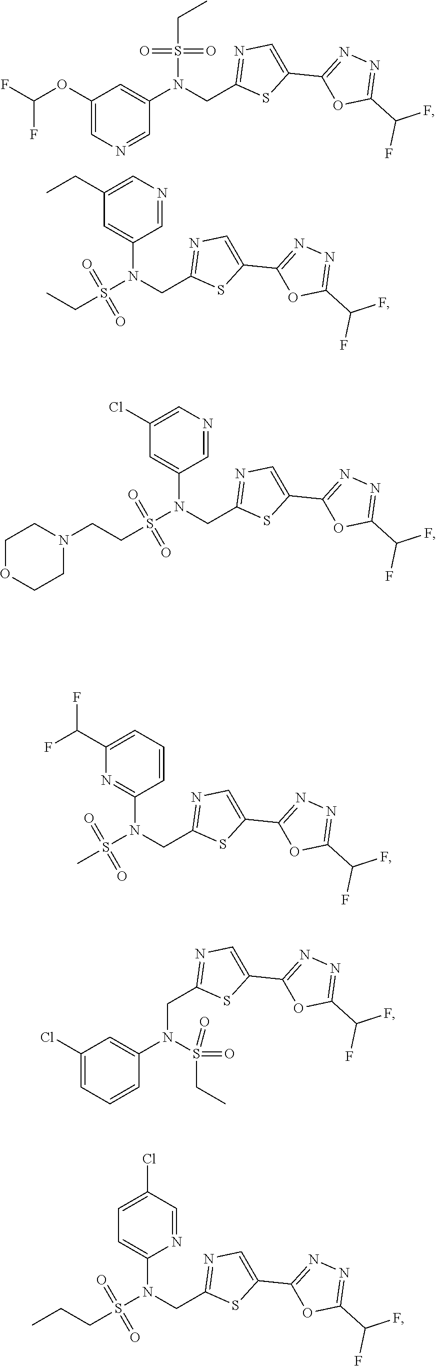
or a salt thereof.

38. The method of claim 16, wherein the selective HDAC6 inhibitor is a compound of Formula: 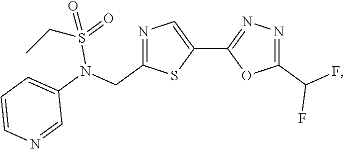 [SMILES: CCS(=O)(=O)N(Cc1ncc(-c2nnc(C(F)F)o2)s1)c1cccnc1]
or a salt thereof.

39-70. (canceled)

71. The method of claim 1, wherein the cells are transfected with:
(i) one or more of the recombinant viral vector to be packaged in the cells into viral particles,
and wherein the cells comprise or are further transfected with:
(ii) one or more helper plasmids comprising a helper gene or genes; and/or
(iii) one or more plasmids comprising one or more genes encoding a structural protein or proteins necessary for viral replication and/or encapsidation.

72-84. (canceled)

85. The method of claim 1, wherein the viral particle titer is increased at least or more than 20%, 25%, 30%, 40%, 50%, 60%, 70%, 80%, 90%, 100%, 200%, 300%, 400%, 500%, 1000% or 5000% compared to viral particle titer under the same conditions but in the absence of the selective HDAC6 inhibitor.

86. (canceled)

87. The method of claim 1, wherein the method further comprises adding to the cell culture one or more additional compounds selected from the group consisting of a caspase inhibitor, a stimulator of interferon genes (STING) inhibitor, and a pan-HDAC inhibitor.

88. A method of producing viral particles and/or increasing viral particle titer, comprising:
transfecting mammalian cells in a cell culture with a recombinant AAV9 vector to be packaged in the cells into viral particles;
adding to the cell culture a fluoroalkyl-oxadiazole derivative; and
culturing the cells in the cell culture.

89. A method of producing viral particles and/or increasing viral particle titer, comprising:
transfecting mammalian cells in a cell culture with a recombinant AAV5 vector to be packaged in the cells into viral particles;
adding to the cell culture a fluoroalkyl-oxadiazole derivative; and
culturing the cells in the cell culture.

90-127. (canceled)

128. A kit comprising (i) a selective HDAC6 inhibitor, (ii) one or more viruses, viral vectors or plasmids, (iii) one or more cells, and/or (iv) cell culture media; or any combination of (i)-(iv).


SEQUENCE LISTING (102 sequences)

SEQ ID NO: 1 - DNA, 7135 bp, synthetic construct
  source 1..7135: mol_type = other DNA
gcccttaagtcatggagaagacccaccttgcagatgtcctcactggggctggcagagccggcaacctgcccaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagccaccatggctgctcctggtgctcctgccgagtacggctacatcagaacagtgctgggccagcagatcctgggacagctggattctagctctctggccctgccttctgaggccaagctgaaactggccggcagttctggaagaggcggccagacagtgaagtccctgcggatccaagaacaggtgcagcagaccctggccagaaagggcagatcttctgtcggcaacggcaacctgcacagaaccagctctgtgcccgagtacgtgtacaatctgcacctggtggaaaacgacttcgtcggcggcagatcccctgtgcctaagacctacgatatgctgaaggccggcaccaccgccacctatgaaggcagatggggaagaggcacagcccagtacagcagccagaaaagcgtggaagagagaagcctgcggcaccctctgcggagactggaaatcagccctgatagcagcccagagagagcccactacacccacagcgactaccagtactcccagagatctcaggccggccacacactgcaccaccaagagtctagaagggccgctctgctggtgcctcctagatacgccagatctgagatcgtgggcgtgtccagagccggcacaacaagcagacagagacacttcgacacctaccaccggcagtatcagcacggcagcgtgtccgataccgtgttcgatagcatccccgccaatcctgctctgctgacataccctagacctggcacctccagatccatgggcaatctgctggaaaaagagaactacctgaccgccggactgaccgtgggacaagttcgacctctggttcctctgcagcccgtgacacagaacagagccagcagaagcagctggcaccagtccagcttccacagcaccagaacactgagagaagctggccctagcgtggccgtggattcttctggtagaagggctcacctgacagttggccaagcagctgcaggcggaagcggaaatctgctgaccgagagaagcaccttcaccgacagccagctgggcaacgccgacatggaaatgacactggaacgggccgtgtccatgctggaagccgatcacatgctgcccagcagaattagcgccgctgccacctttatccagcacgagtgcttccagaagtctgaggcccggaagagagtgaaccagctgagaggcatcctgaagctgctgcagctcctgaaggtgcagaacgaggatgtgcagagggctgtgtgtggggccctgagaaatctggtgttcgaggacaacgacaacaagctggaagtggccgagctgaacggcgtgccaagactgctgcaggttctgaaacagacccgcgacctggaaacaaagaagcagatcaccggcctgctctggaacctgagcagcaacgacaagctgaagaacctgatgatcacagaggccctgctgaccctgacagagaacatcatcatccctttcagcggctggcccgagggcgattaccctaaagctaatggcctgctggacttcgacatcttctacaacgtgaccggctgcctgagaaacatgtctagcgctggcgccgatggcagaaaggccatgagaagatgtgacggcctgatcgacagcctggtgcactatgtgcggggcacaatcgccgattaccagcctgatgataaggccaccgagaactgcgtgtgcatcctgcacaacctgagctaccagctggaagcagagctgcccgagaagtacagccagaacatctacatccagaaccggaacatccagaccgacaacaacaagagcatcggctgcttcggcagccgcagccggaaagtgaaagaacagtaccaggacgtgcccatgcctgaggaaaagtctaaccccaaaggcgtggaatggctgtggcacagcatcgtgatccggatgtacctgagcctgatcgccaagagcgtgcggaattacacccaagaggcatctctgggcgccctgcagaatctgacagcaggatctggccctatgcctacctctgtggctcagaccgtggtgcagaaagagtctggcctgcagcacacccggaagatgctgcatgtgggagatcccagcgtgaagaaaaccgccatcagcctgctgagaaacctgagccggaatctgtctctgcagaatgagatcgccaaagagacactgcccgacctggtgtctatcatccctgacaccgtgcctagcaccgacctgctgattgagacaacagccagcgcctgctacaccctgaacaacatcattcagaactcctaccagaacgcccgcgatctgctgaacacaggcggcatccagaaaatcatggccatctctgccggcgacgcctacgcctctaacaaggcctctaaagccgccagcgtgctgctgtattctctgtgggcccataccgagctgcaccatgcctataagaaggcccagttcaaaaagaccgacttcgtgaacagccggaccgccaaggcctaccactctctgaaagattaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattc

SEQ ID NO: 2 - DNA, 11635 bp, synthetic construct
  source 1..11635: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 3 - DNA, 7025 bp, synthetic construct
  source 1..7025: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtagttaatgattaacccgccatgctacttatctacgtagccatgctctaggaagatcggaattcgccctattagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaaggcgcgccataacttcgttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 6980 bp, synthetic construct
  source 1..6980: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtagttaatgattaacccgccatgctacttatctacgtagccatgctctaggaagatcggaattcgccctattagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaaccgtgtattcgtgtcagtaaaatactgcctcttgtggtttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 7025 bp, synthetic construct
  source 1..7025: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtagttaatgattaacccgccatgctacttatctacgtagccatgctctaggaagatcggaattcgccctattagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaagggctccggccagaatcagcaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccacaattccacctacctgggcgcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaaggcgcgccataacttcgttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 6 - DNA, 7025 bp, synthetic construct
  source 1..7025: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtagttaatgattaacccgccatgctacttatctacgtagccatgctctaggaagatcggaattcgccctattagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccatatgatgaccaccgccagagcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaaggcgcgccataacttcgttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 7025 bp, synthetic construct
  source 1..7025: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtagttaatgattaacccgccatgctacttatctacgtagccatgctctaggaagatcggaattcgccctattagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccacaattccacctacctgggcgcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaaggcgcgccataacttcgttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 7025 bp, synthetic construct
  source 1..7025: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtagttaatgattaacccgccatgctacttatctacgtagccatgctctaggaagatcggaattcgccctattagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccattgtagcactagcatcagggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaaggcgcgccataacttcgttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 7025 bp, synthetic construct
  source 1..7025: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtagttaatgattaacccgccatgctacttatctacgtagccatgctctaggaagatcggaattcgccctattagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccaggctaattatggtcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaaggcgcgccataacttcgttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - protein, 621 aa, unidentified
  source 1..621: note = Unclassified Dependoparvovirus adeno-associated virus
TAGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 11 - protein, 736 aa, unidentified
  source 1..736: note = Unclassified Dependoparvovirus adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - protein, 599 aa, unidentified
  source 1..599: note = Unclassified Dependoparvovirus adeno-associated virus 9
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 534 aa, unidentified
  source 1..534: note = Unclassified Dependoparvovirus adeno-associated virus 9
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 197 aa, unidentified
  source 1..197: note = Unclassified Dependoparvovirus adeno-associated virus 9
LATQSQSQTLNQSENLPQPPQVWDLLQWLQVVAHQWQTITKVPMEWVVPREIGIAIPNGWGTESSPPAPEPGPCPPTTITSTSKSPTAHLEDLQMTTPTSATAPPGGILTSTDSTATSHHVTGSDSSTTTGDSGLSDSTSSSSTFRSKRLRTTMESRPSPITLPARSRSSRTQTISSRTCSGRLTRAASRRSQRTFS

SEQ ID NO: 15 - protein, 7 aa, unidentified
  source 1..7: note = Unclassified Dependoparvovirus adeno-associated virus 9
NGSGQNQ

SEQ ID NO: 16 - protein, 6 aa, unidentified
  source 1..6: note = Unclassified Dependoparvovirus adeno-associated virus 9
NNSEFA

SEQ ID NO: 17 - protein, 5 aa, unidentified
  source 1..5: note = Unclassified Dependoparvovirus adeno-associated virus 9
GRDNV

SEQ ID NO: 18 - protein, 14 aa, unidentified
  source 1..14: note = Unclassified Dependoparvovirus adeno-associated virus 9
ATNHQSAQAQAQTG

SEQ ID NO: 19 - protein, 724 aa, unidentified
  source 1..724: note = Unclassified Dependoparvovirus adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 20 - protein, 588 aa, unidentified
  source 1..588: note = Unclassified Dependoparvovirus adeno-associated virus 5
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 21 - protein, 532 aa, unidentified
  source 1..532: note = Unclassified Dependoparvovirus adeno-associated virus 5
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 22 - protein, 201 aa, unidentified
  source 1..201: note = Unclassified Dependoparvovirus adeno-associated virus 5
LDPADPSSCKSQPNQPQVWELIQCLREVAAHWATITKVPMEWAMPREIGIAIPRGWGTESSPSPPEPGCCPATTTTSTERSKAAPSTEATPTPTLDTAPPGGTLTLTASTATGAPETGKDSSTTTGASDPGPSESKSSTFKSKRSRCRTPPPPSPTTSPPPSKCLRTTTTSCPTSSATGPRDACRPSLRRSLRCRSTVTRR

SEQ ID NO: 23 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNSTYLGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMMTTARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNSTYLGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCSTSIRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 27 - protein, 7 aa, synthetic construct
KGSGQNQ

SEQ ID NO: 28 - protein, 7 aa, synthetic construct
NASGQNQ

SEQ ID NO: 29 - protein, 7 aa, synthetic construct
NGTGQNQ

SEQ ID NO: 30 - protein, 7 aa, synthetic construct
NGSGLNQ

SEQ ID NO: 31 - protein, 7 aa, synthetic construct
ANDNKLI

SEQ ID NO: 32 - protein, 7 aa, synthetic construct
VNDNKVI

SEQ ID NO: 33 - protein, 7 aa, synthetic construct
NGSGQNH

SEQ ID NO: 34 - protein, 7 aa, synthetic construct
ANDNKVI

SEQ ID NO: 35 - protein, 6 aa, synthetic construct
ENTVSI

SEQ ID NO: 36 - protein, 6 aa, synthetic construct
QTLFNS

SEQ ID NO: 37 - protein, 6 aa, synthetic construct
NSTYLG

SEQ ID NO: 38 - protein, 6 aa, synthetic construct
GSILTH

SEQ ID NO: 39 - protein, 6 aa, synthetic construct
MMTTAR

SEQ ID NO: 40 - protein, 6 aa, synthetic construct
CSTSIR

SEQ ID NO: 41 - protein, 14 aa, synthetic construct
ATNHNSTYLGAQTG

SEQ ID NO: 42 - protein, 14 aa, synthetic construct
ATNHMMTTARAQTG

SEQ ID NO: 43 - protein, 14 aa, synthetic construct
ATNHCSTSIRAQTG

SEQ ID NO: 44 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 14 aa, synthetic construct
XDVQXXPGFXXXXX

SEQ ID NO: 46 - protein, 14 aa, synthetic construct
ADVQQXPGFXXXTX

SEQ ID NO: 47 - protein, 14 aa, synthetic construct
XDVQXXPGFXAQXX

SEQ ID NO: 48 - protein, 14 aa, synthetic construct
XDVQXXPGFXAEXX

SEQ ID NO: 49 - protein, 14 aa, synthetic construct
XDVQXXPGFXPQXX

SEQ ID NO: 50 - protein, 14 aa, synthetic construct
XDVQXXPGFXPEXX

SEQ ID NO: 51 - protein, 14 aa, synthetic construct
ADVQQXPGFXAQTX

SEQ ID NO: 52 - protein, 14 aa, synthetic construct
ADVQQXPGFXAETX

SEQ ID NO: 53 - protein, 14 aa, synthetic construct
ADVQQXPGFXPQTX

SEQ ID NO: 54 - protein, 14 aa, synthetic construct
ADVQQXPGFXPETX

SEQ ID NO: 55 - protein, 14 aa, synthetic construct
XDVQXXPGFQAQXX

SEQ ID NO: 56 - protein, 14 aa, synthetic construct
ADVQQXPGFQAQTX

SEQ ID NO: 57 - protein, 14 aa, synthetic construct
XDVQXLPGFXXXXE

SEQ ID NO: 58 - protein, 14 aa, synthetic construct
ADVQQLPGFXXXTE

SEQ ID NO: 59 - protein, 14 aa, synthetic construct
XDVQXXPGFQXXXX

SEQ ID NO: 60 - protein, 14 aa, synthetic construct
ADVQQXPGFQXXTX

SEQ ID NO: 61 - protein, 14 aa, synthetic construct
ADVQQLPGFQAQTE

SEQ ID NO: 62 - protein, 14 aa, synthetic construct
ADVQQHPGFQAQTE

SEQ ID NO: 63 - protein, 14 aa, synthetic construct
ADVQQVPGFQAQTM

SEQ ID NO: 64 - protein, 14 aa, synthetic construct
ADVQQVPGFQAQTQ

SEQ ID NO: 65 - protein, 14 aa, synthetic construct
ADVQQLPGFGAQTE

SEQ ID NO: 66 - protein, 14 aa, synthetic construct
ADVQQLPGFRPETE

SEQ ID NO: 67 - protein, 14 aa, synthetic construct
ADVQQLPGFNAQTE

SEQ ID NO: 68 - protein, 7 aa, synthetic construct
RGDAARL

SEQ ID NO: 69 - protein, 7 aa, synthetic construct
SHVRGDL

SEQ ID NO: 70 - protein, 7 aa, synthetic construct
VVSSGAR

SEQ ID NO: 71 - protein, 4 aa, synthetic construct
VRGD

SEQ ID NO: 72 - protein, 7 aa, synthetic construct
RTDLKGL

SEQ ID NO: 73 - protein, 7 aa, synthetic construct
YPSTGSG

SEQ ID NO: 74 - protein, 8 aa, synthetic construct
FAGSLTRA

SEQ ID NO: 75 - protein, 7 aa, synthetic construct
DRTLTTR

SEQ ID NO: 76 - protein, 7 aa, synthetic construct
RIAGRDV

SEQ ID NO: 77 - protein, 7 aa, synthetic construct
SLGSGVR

SEQ ID NO: 78 - protein, 7 aa, synthetic construct
PQYGRGG

SEQ ID NO: 79 - protein, 7 aa, synthetic construct
LQVSRVS

SEQ ID NO: 80 - protein, 7 aa, synthetic construct
VRSYSSN

SEQ ID NO: 81 - protein, 7 aa, synthetic construct
TMRVGSL

SEQ ID NO: 82 - protein, 7 aa, synthetic construct
GAYSRGV

SEQ ID NO: 83 - protein, 7 aa, synthetic construct
LRGGSLG

SEQ ID NO: 84 - protein, 7 aa, synthetic construct
VYGTGVR

SEQ ID NO: 85 - protein, 7 aa, synthetic construct
DKLIIVS

SEQ ID NO: 86 - protein, 7 aa, synthetic construct
AEDRTKL

SEQ ID NO: 87 - protein, 7 aa, synthetic construct
LSASASL

SEQ ID NO: 88 - protein, 7 aa, synthetic construct
LADQTKL

SEQ ID NO: 89 - protein, 7 aa, synthetic construct
LLLKLQE

SEQ ID NO: 90 - protein, 7 aa, synthetic construct
ELPVKTG

SEQ ID NO: 91 - protein, 7 aa, synthetic construct
LDLKVVG

SEQ ID NO: 92 - protein, 5 aa, synthetic construct
RDAVL

SEQ ID NO: 93 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFQAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 94 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQHPGFQAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 95 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHVRGDLSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 96 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGVVSSGARSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 97 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDAARLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 98 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQVPGFQAQTMWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 99 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQVPGFQAQTQWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 100 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFGAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 101 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFRPETEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 102 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFNAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL